SEQUENCE LISTING (82 sequences)

SEQ ID NO: 1 - DNA, 4718 bp, Adeno-associated virus
ttgcccactccctctctgcgcgctcgctcgctcggtggggcctgcggaccaaaggtccgcagacggcagagctctgctctgccggccccaccgagcgagcgagcgcgcagagagggagtgggcaactccatcactaggggtaatcgcgaagcgcctcccacgctgccgcgtcagcgctgacgtaaattacgtcataggggagtggtcctgtattagctgtcacgtgagtgcttttgcgacattttgcgacaccacgtggccatttagggtatatatggccgagtgagcgagcaggatctccattttgaccgcgaaatttgaacgagcagcagccatgccgggcttctacgagatcgtgatcaaggtgccgagcgacctggacgagcacctgccgggcatttctgactcgtttgtgagctgggtggccgagaaggaatgggagctgcccccggattctgacatggatctgaatctgattgagcaggcacccctgaccgtggccgagaagctgcagcgcgacttcctggtccaatggcgccgcgtgagtaaggccccggaggccctcttctttgttcagttcgagaagggcgagtcctacttccacctccatattctggtggagaccacgggggtcaaatccatggtgctgggccgcttcctgagtcagattagggacaagctggtgcagaccatctaccgcgggatcgagccgaccctgcccaactggttcgcggtgaccaagacgcgtaatggcgccggaggggggaacaaggtggtggacgagtgctacatccccaactacctcctgcccaagactcagcccgagctgcagtgggcgtggactaacatggaggagtatataagcgcctgtttgaacctggccgagcgcaaacggctcgtggcgcagcacctgacccacgtcagccagacccaggagcagaacaaggagaatctgaaccccaattctgacgcgcctgtcatccggtcaaaaacctccgcgcgctacatggagctggtcgggtggctggtggaccggggcatcacctccgagaagcagtggatccaggaggaccaggcctcgtacatctccttcaacgccgcttccaactcgcggtcccagatcaaggccgctctggacaatgccggcaagatcatggcgctgaccaaatccgcgcccgactacctggtaggccccgctccgcccgcggacattaaaaccaaccgcatctaccgcatcctggagctgaacggctacgaacctgcctacgccggctccgtctttctcggctgggcccagaaaaggttcgggaagcgcaacaccatctggctgtttgggccggccaccacgggcaagaccaacatcgcggaagccatcgcccacgccgtgcccttctacggctgcgtcaactggaccaatgagaactttcccttcaatgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccacccccgtgatcgtcacctccaacaccaacatgtgcgccgtgattgacgggaacagcaccaccttcgagcaccagcagccgttgcaggaccggatgttcaaatttgaactcacccgccgtctggagcatgactttggcaaggtgacaaagcaggaagtcaaagagttcttccgctgggcgcaggatcacgtgaccgaggtggcgcatgagttctacgtcagaaagggtggagccaacaaaagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaagacatgcgagagaatgaatcagaatttcaacatttgcttcacgcacgggacgagagactgttcagagtgcttccccggcgtgtcagaatctcaaccggtcgtcagaaagaggacgtatcggaaactctgtgccattcatcatctgctggggcgggctcccgagattgcttgctcggcctgcgatctggtcaacgtggacctggatgactgtgtttctgagcaataaatgacttaaaccaggtatggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaattacgtgttaatcaataaaccggttgattcgtttcagttgaactttggtctcctgtccttcttatcttatcggttaccatggttatagcttacacattaactgcttggttgcgcttcgcgataaaagacttacgtcatcgggttacccctagtgatggagttgcccactccctctctgcgcgctcgctcgctcggtggggcctgcggaccaaaggtccgcagacggcagagctctgctctgccggccccaccgagcgagcgagcgcgcagagagggagtgggcaa

SEQ ID NO: 2 - protein, 736 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 3 - DNA, 2208 bp, Adeno-associated virus
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 4 - protein, 735 aa, Adeno-associated virus [sequence omitted]

SEQ ID NO: 5 - DNA, 4726 bp, Adeno-associated virus
ttggccactccctctatgcgcactcgctcgctcggtggggcctggcgaccaaaggtcgccagacggacgtgctttgcacgtccggccccaccgagcgagcgagtgcgcatagagggagtggccaactccatcactagaggtatggcagtgacgtaacgcgaagcgcgcgaagcgagaccacgcctaccagctgcgtcagcagtcaggtgacccttttgcgacagtttgcgacaccacgtggccgctgagggtatatattctcgagtgagcgaaccaggagctccattttgaccgcgaaatttgaacgagcagcagccatgccggggttctacgagattgtcctgaaggtcccgagtgacctggacgagcgcctgccgggcatttctaactcgtttgttaactgggtggccgagaaggaatgggacgtgccgccggattctgacatggatccgaatctgattgagcaggcacccctgaccgtggccgaaaagcttcagcgcgagttcctggtggagtggcgccgcgtgagtaaggccccggaggccctcttttttgtccagttcgaaaagggggagacctacttccacctgcacgtgctgattgagaccatcggggtcaaatccatggtggtcggccgctacgtgagccagattaaagagaagctggtgacccgcatctaccgcggggtcgagccgcagcttccgaactggttcgcggtgaccaaaacgcgaaatggcgccgggggcgggaacaaggtggtggacgactgctacatccccaactacctgctccccaagacccagcccgagctccagtgggcgtggactaacatggaccagtatttaagcgcctgtttgaatctcgcggagcgtaaacggctggtggcgcagcatctgacgcacgtgtcgcagacgcaggagcagaacaaagagaatcagaaccccaattctgacgcgccggtcatcaggtcaaaaacctcagccaggtacatggagctggtcgggtggctggtggaccgcgggatcacgtcagaaaagcaatggattcaggaggaccaggcctcgtacatctccttcaacgccgcctccaactcgcggtcccagatcaaggccgcgctggacaatgcctccaagatcatgagcctgacaaagacggctccggactacctggtgggcagcaacccgccggaggacattaccaaaaatcggatctaccaaatcctggagctgaacgggtacgatccgcagtacgcggcctccgtcttcctgggctgggcgcaaaagaagttcgggaagaggaacaccatctggctctttgggccggccacgacgggtaaaaccaacatcgcggaagccatcgcccacgccgtgcccttctacggctgcgtaaactggaccaatgagaactttcccttcaacgattgcgtcgacaagatggtgatctggtgggaggagggcaagatgacggccaaggtcgtggagagcgccaaggccattctgggcggaagcaaggtgcgcgtggaccaaaagtgcaagtcatcggcccagatcgaacccactcccgtgatcgtcacctccaacaccaacatgtgcgccgtgattgacgggaacagcaccaccttcgagcatcagcagccgctgcaggaccggatgtttgaatttgaacttacccgccgtttggaccatgactttgggaaggtcaccaaacaggaagtaaaggactttttccggtgggcttccgatcacgtgactgacgtggctcatgagttctacgtcagaaagggtggagctaagaaacgccccgcctccaatgacgcggatgtaagcgagccaaaacgggagtgcacgtcacttgcgcagccgacaacgtcagacgcggaagcaccggcggactacgcggacaggtaccaaaacaaatgttctcgtcacgtgggcatgaatctgatgctttttccctgtaaaacatgcgagagaatgaatcaaatttccaatgtctgttttacgcatggtcaaagagactgtggggaatgcttccctggaatgtcagaatctcaacccgtttctgtcgtcaaaaagaagacttatcagaaactgtgtccaattcatcatatcctgggaagggcacccgagattgcctgttcggcctgcgatttggccaatgtggacttggatgactgtgtttctgagcaataaatgacttaaaccaggtatggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagggggctgtagatcagtctcctcaggaaccggactcatcatctggtgttggcaaatcgggcaaacagcctgccagaaaaagactaaatttcggtcagactggagactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttagaggggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactggaactgtcaatcatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtgaatcctggttaatcaataaaccgtttaattcgtttcagttgaactttggctcttgtgcacttctttatctttatcttgtttccatggctactgcgtagataagcagcggcctgcggcgcttgcgcttcgcggtttacaactgctggttaatatttaactctcgccatacctctagtgatggagttggccactccctctatgcgcactcgctcgctcggtggggcctggcgaccaaaggtcgccagacggacgtgctttgcacgtccggccccaccgagcgagcgagtgcgcatagagggagtggccaa

SEQ ID NO: 6 - protein, 736 aa, Adeno-associated virus
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 7 - DNA, 4768 bp, Adeno-associated virus
  modified_base (3009)..(3009): unknown
ttggccactccctctatgcgcgctcgctcactcactcggccctggagaccaaaggtctccagactgccggcctctggccggcagggccgagtgagtgagcgagcgcgcatagagggagtggccaactccatcatctaggtttgcccactgacgtcaatgtgacgtcctagggttagggaggtccctgtattagcagtcacgtgagtgtcgtatttcgcggagcgtagcggagcgcataccaagctgccacgtcacagccacgtggtccgtttgcgacagtttgcgacaccatgtggtcaggagggtatataaccgcgagtgagccagcgaggagctccattttgcccgcgaattttgaacgagcagcagccatgccggggttctacgagatcgtgctgaaggtgcccagcgacctggacgagcacctgcccggcatttctgactcttttgtgagctgggtggccgagaaggaatgggagctgccgccggattctgacatggacttgaatctgattgagcaggcacccctgaccgtggccgaaaagctgcaacgcgagttcctggtcgagtggcgccgcgtgagtaaggccccggaggccctcttctttgtccagttcgagaagggggacagctacttccacctgcacatcctggtggagaccgtgggcgtcaaatccatggtggtgggccgctacgtgagccagattaaagagaagctggtgacccgcatctaccgcggggtcgagccgcagcttccgaactggttcgcggtgaccaagacgcgtaatggcgccggaggcgggaacaaggtggtggacgactgctacatccccaactacctgctccccaagacccagcccgagctccagtgggcgtggactaacatggaccagtatataagcgcctgtttgaatctcgcggagcgtaaacggctggtggcgcagcatctgacgcacgtgtcgcagacgcaggagcagaacaaggaaaaccagaaccccaattctgacgcgccggtcatcaggtcaaaaacctccgccaggtacatggagctggtcgggtggctggtggaccgcgggatcacgtcagaaaagcaatggatccaggaggaccaggcgtcctacatctccttcaacgccgcctccaactcgcggtcacaaatcaaggccgcgctggacaatgcctccaaaatcatgagcctgacaaagacggctccggactacctggtgggccagaacccgccggaggacatttccagcaaccgcatctaccgaatcctcgagatgaacgggtacgatccgcagtacgcggcctccgtcttcctgggctgggcgcaaaagaagttcgggaagaggaacaccatctggctctttgggccggccacgacgggtaaaaccaacatcgcggaagccatcgcccacgccgtgcccttctacggctgcgtgaactggaccaatgagaactttccgttcaacgattgcgtcgacaagatggtgatctggtgggaggagggcaagatgacggccaaggtcgtagagagcgccaaggccatcctgggcggaagcaaggtgcgcgtggaccaaaagtgcaagtcatcggcccagatcgacccaactcccgtgatcgtcacctccaacaccaacatgtgcgcggtcatcgacggaaactcgaccaccttcgagcaccaacaaccactccaggaccggatgttcaagttcgagctcaccaagcgcctggagcacgactttggcaaggtcaccaagcaggaagtcaaagactttttccggtgggcgtcagatcacgtgaccgaggtgactcacgagttttacgtcagaaagggtggagctagaaagaggcccgcccccaatgacgcagatataagtgagcccaagcgggcctgtccgtcagttgcgcagccatcgacgtcagacgcggaagctccggtggactacgcggacaggtaccaaaacaaatgttctcgtcacgtgggtatgaatctgatgctttttccctgccggcaatgcgagagaatgaatcagaatgtggacatttgcttcacgcacggggtcatggactgtgccgagtgcttccccgtgtcagaatctcaacccgtgtctgtcgtcagaaagcggacgtatcagaaactgtgtccgattcatcacatcatggggagggcgcccgaggtggcctgctcggcctgcgaactggccaatgtggacttggatgactgtgacatggaacaataaatgactcaaaccagatatgactgacggttaccttccagattggctagaggacaacctctctgaaggcgttcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgcggagttccagcagcggcttcagggcgacacaccgtttgggggcaacctcggcagagcagtcttccaggccaaaaagagggttcttgaacctcttggtctggttgagcaagcgggtgagacggctcctggaaagaagagaccgttgattgaatccccccagcagcccgactcctccacgggtatcggcaaaaaaggcaagcagccggctaaaaagaagctcgttttcgaagacgaaactggagcaggcgacggaccccctgagggatcaacttccggagccatgtctgatgacagtgagatgcgtgcagcagctggcggagctgcagtcgagggsggacaaggtgccgatggagtgggtaatgcctcgggtgattggcattgcgattccacctggtctgagggccacgtcacgaccaccagcaccagaacctgggtcttgcccacctacaacaaccacctntacaagcgactcggagagagcctgcagtccaacacctacaacggattctccaccccctggggatactttgacttcaaccgcttccactgccacttctcaccacgtgactggcagcgactcatcaacaacaactggggcatgcgacccaaagccatgcgggtcaaaatcttcaacatccaggtcaaggaggtcacgacgtcgaacggcgagacaacggtggctaataaccttaccagcacggttcagatctttgcggactcgtcgtacgaactgccgtacgtgatggatgcgggtcaagagggcagcctgcctccttttcccaacgacgtctttatggtgccccagtacggctactgtggactggtgaccggcaacacttcgcagcaacagactgacagaaatgccttctactgcctggagtactttccttcgcagatgctgcggactggcaacaactttgaaattacgtacagttttgagaaggtgcctttccactcgatgtacgcgcacagccagagcctggaccggctgatgaaccctctcatcgaccagtacctgtggggactgcaatcgaccaccaccggaaccaccctgaatgccgggactgccaccaccaactttaccaagctgcggcctaccaacttttccaactttaaaaagaactggctgcccgggccttcaatcaagcagcagggcttctcaaagactgccaatcaaaactacaagatccctgccaccgggtcagacagtctcatcaaatacgagacgcacagcactctggacggaagatggagtgccctgacccccggacctccaatggccacggctggacctgcggacagcaagttcagcaacagccagctcatctttgcggggcctaaacagaacggcaacacggccaccgtacccgggactctgatcttcacctctgaggaggagctggcagccaccaacgccaccgatacggacatgtggggcaacctacctggcggtgaccagagcaacagcaacctgccgaccgtggacagactgacagccttgggagccgtgcctggaatggtctggcaaaacagagacatttactaccagggtcccatttgggccaagattcctcataccgatggacactttcacccctcaccgctgattggtgggtttgggctgaaacacccgcctcctcaaatttttatcaagaacaccccggtacctgcgaatcctgcaacgaccttcagctctactccggtaaactccttcattactcagtacagcactggccaggtgtcggtgcagattgactgggagatccagaaggagcggtccaaacgctggaaccccgaggtccagtttacctccaactacggacagcaaaactctctgttgtgggctcccgatgcggctgggaaatacactgagcctagggctatcggtacccgctacctcacccaccacctgtaataacctgttaatcaataaaccggtttattcgtttcagttgaactttggtctccgtgtccttcttatcttatctcgtttccatggctactgcgtacataagcagcggcctgcggcgcttgcgcttcgcggtttacaactgccggttaatcagtaacttctggcaaaccatgatgatggagttggccactccctctatgcgcgctcgctcactcactcggccctggagaccaaaggtctccagactgccggcctctggccggcagggccgagtgagtgagcgagcgcgcatagagggagtggccaa

SEQ ID NO: 8 - DNA, 2205 bp, Adeno-associated virus
atgactgacggttaccttccagattggctagaggacaacctctctgaaggcgttcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgcggagttccagcagcggcttcagggcgacacatcgtttgggggcaacctcggcagagcagtcttccaggccaaaaagagggttcttgaacctcttggtctggttgagcaagcgggtgagacggctcctggaaagaagagaccgttgattgaatccccccagcagcccgactcctccacgggtatcggcaaaaaaggcaagcagccggctaaaaagaagctcgttttcgaagacgaaactggagcaggcgacggaccccctgagggatcaacttccggagccatgtctgatgacagtgagatgcgtgcagcagctggcggagctgcagtcgagggcggacaaggtgccgatggagtgggtaatgcctcgggtgattggcattgcgattccacctggtctgagggccacgtcacgaccaccagcaccagaacctgggtcttgcccacctacaacaaccacctctacaagcgactcggagagagcctgcagtccaacacctacaacggattctccaccccctggggatactttgacttcaaccgcttccactgccacttctcaccacgtgactggcagcgactcatcaacaacaactggggcatgcgacccaaagccatgcgggtcaaaatcttcaacatccaggtcaaggaggtcacgacgtcgaacggcgagacaacggtggctaataaccttaccagcacggttcagatctttgcggactcgtcgtacgaactgccgtacgtgatggatgcgggtcaagagggcagcctgcctccttttcccaacgacgtctttatggtgccccagtacggctactgtggactggtgaccggcaacacttcgcagcaacagactgacagaaatgccttctactgcctggagtactttccttcgcagatgctgcggactggcaacaactttgaaattacgtacagttttgagaaggtgcctttccactcgatgtacgcgcacagccagagcctggaccggctgatgaaccctctcatcgaccagtacctgtggggactgcaatcgaccaccaccggaaccaccctgaatgccgggactgccaccaccaactttaccaagctgcggcctaccaacttttccaactttaaaaagaactggctgcccgggccttcaatcaagcagcagggcttctcaaagactgccaatcaaaactacaagatccctgccaccgggtcagacagtctcatcaaatacgagacgcacagcactctggacggaagatggagtgccctgacccccggacctccaatggccacggctggacctgcggacagcaagttcagcaacagccagctcatctttgcggggcctaaacagaacggcaacacggccaccgtacccgggactctgatcttcacctctgaggaggagctggcagccaccaacgccaccgatacggacatgtggggcaacctacctggcggtgaccagagcaacagcaacctgccgaccgtggacagactgacagccttgggagccgtgcctggaatggtctggcaaaacagagacatttactaccagggtcccatttgggccaagattcctcataccgatggacactttcacccctcaccgctgattggtgggtttgggctgaaacacccgcctcctcaaatttttatcaagaacaccccggtacctgcgaatcctgcaacgaccttcagctctactccggtaaactccttcattactcagtacagcactggccaggtgtcggtgcagattgactgggagatccagaaggagcggtccaaacgctggaaccccgaggtccagtttacctccaactacggacagcaaaactctctgttgtgggctcccgatgcggctgggaaatacactgagcctagggctatcggtacccgctacctcacccaccacctgtaa

SEQ ID NO: 9 - protein, 734 aa, Adeno-associated virus
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 10 - DNA, 2172 bp, Adeno-associated virus
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctt

SEQ ID NO: 11 - protein, 724 aa, Adeno-associated virus
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 12 - DNA, 4683 bp, Adeno-associated virus
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagcgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgacttcctggtccagtggcgccgcgtgagtaaggccccggaggccctcttctttgttcagttcgagaagggcgagtcctacttccacctccatattctggtggagaccacgggggtcaaatccatggtgctgggccgcttcctgagtcagattagggacaagctggtgcagaccatctaccgcgggatcgagccgaccctgcccaactggttcgcggtgaccaagacgcgtaatggcgccggaggggggaacaaggtggtggacgagtgctacatccccaactacctcctgcccaagactcagcccgagctgcagtgggcgtggactaacatggaggagtatataagcgcgtgtttaaacctggccgagcgcaaacggctcgtggcgcacgacctgacccacgtcagccagacccaggagcagaacaaggagaatctgaaccccaattctgacgcgcctgtcatccggtcaaaaacctccgcacgctacatggagctggtcgggtggctggtggaccggggcatcacctccgagaagcagtggatccaggaggaccaggcctcgtacatctccttcaacgccgcctccaactcgcggtcccagatcaaggccgctctggacaatgccggcaagatcatggcgctgaccaaatccgcgcccgactacctggtaggccccgctccgcccgccgacattaaaaccaaccgcatttaccgcatcctggagctgaacggctacgaccctgcctacgccggctccgtctttctcggctgggcccagaaaaggttcggaaaacgcaacaccatctggctgtttgggccggccaccacgggcaagaccaacatcgcggaagccatcgcccacgccgtgcccttctacggctgcgtcaactggaccaatgagaactttcccttcaacgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgatcccacccccgtgatcgtcacctccaacaccaacatgtgcgccgtgattgacgggaacagcaccaccttcgagcaccagcagccgttgcaggaccggatgttcaaatttgaactcacccgccgtctggagcatgactttggcaaggtgacaaagcaggaagtcaaagagttcttccgctgggcgcaggatcacgtgaccgaggtggcgcatgagttctacgtcagaaagggtggagccaacaagagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaaaacatgcgagagaatgaatcagaatttcaacatttgcttcacgcacgggaccagagactgttcagaatgtttccccggcgtgtcagaatctcaaccggtcgtcagaaagaggacgtatcggaaactctgtgccattcatcatctgctggggcgggctcccgagattgcttgctcggcctgcgatctggtcaacgtggatctggatgactgtgtttctgagcaataaatgacttaaaccaggtatggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaattgtgtgttaatcaataaaccggttaattcgtgtcagttgaactttggtctcatgtcgttattatcttatctggtcaccatagcaaccggttacacattaactgcttagttgcgcttcgcgaatacccctagtgatggagttgcccactccctctatgcgcgctcgctcgctcggtggggccggcagagcagagctctgccgtctgcggacctttggtccgcaggccccaccgagcgagcgagcgcgcatagagggagtgggcaa

SEQ ID NO: 13 - DNA, 2211 bp, Adeno-associated virus
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 14 - protein, 736 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 15 - DNA, 2214 bp, Adeno-associated virus
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcatttgggggcaacctcgggcgagcagtcttccaggccaagaagcgggttctcgaacctctcggtctggttgaggaaggcgctaagacggctcctgcaaagaagagaccggtagagccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcattaccaccagcacccgaacctgggccctgcccacctacaacaaccacctctacaagcaaatctccagtgaaactgcaggtagtaccaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattcctcagtacggctacctgactctcaacaatggcagtcagtctgtgggacgttcctccttctactgcctggagtacttcccctctcagatgctgagaacgggcaacaactttgagttcagctacagcttcgaggacgtgcctttccacagcagctacgcacacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctcctcagatcctgatcaagaacactcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacctccaactttgaaaagcagactggtgtggactttgccgttgacagccagggtgtttactctgagcctcgccctattggcactcgttacctcacccgtaatctgtaa

SEQ ID NO: 16 - DNA, 4721 bp, Adeno-associated virus
ttggccactccctctatgcgcgctcgctcgctcggtggggcctgcggaccaaaggtccgcagacggcagagctctgctctgccggccccaccgagcgagcgagcgcgcatagagggagtggccaactccatcactaggggtaccgcgaagcgcctcccacgctgccgcgtcagcgctgacgtaaatcacgtcataggggagtggtcctgtattagctgtcacgtgagtgcttttgcgacattttgcgacaccacgtggccatttgaggtatatatggccgagtgagcgagcaggatctccattttgaccgcgaaatttgaacgagcagcagccatgccgggtttctacgagatcgtgatcaaggtgccgagcgacctggacgagcacctgccgggcatttctgactcgtttgtgaactgggtggccgagaaggaatgggagctgcccccggattctgacatggatctgaatctgatcgagcaggcacccctgaccgtggccgagaagctgcagcgcgacttcctggtccaatggcgccgcgtgagtaaggccccggaggccctgttctttgttcagttcgagaagggcgagagctacttccaccttcacgttctggtggagaccacgggggtcaagtccatggtgctaggccgcttcctgagtcagattcgggagaagctggtccagaccatctaccgcggggtcgagcccacgctgcccaactggttcgcggtgaccaagacgcgtaatggcgccggcggggggaacaaggtggtggacgagtgctacatccccaactacctcctgcccaagacccagcccgagctgcagtgggcgtggactaacatggaggagtatataagcgcgtgtttgaacctggccgaacgcaaacggctcgtggcgcagcacctgacccacgtcagccagacgcaggagcagaacaaggagaatctgaaccccaattctgacgcgcccgtgatcaggtcaaaaacctccgcgcgctacatggagctggtcgggtggctggtggaccggggcatcacctccgagaagcagtggatccaggaggaccaggcctcgtacatctccttcaacgccgcctccaactcgcggtcccagatcaaggccgcgctggacaatgccggcaagatcatggcgctgaccaaatccgcgcccgactacctggtggggccctcgctgcccgcggacattaaaaccaaccgcatctaccgcatcctggagctgaacgggtacgatcctgcctacgccggctccgtctttctcggctgggcccagaaaaagttcgggaagcgcaacaccatctggctgtttgggcccgccaccaccggcaagaccaacattgcggaagccatcgcccacgccgtgcccttctacggctgcgtcaactggaccaatgagaactttcccttcaacgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccacccccgtgatcgtcacctccaacaccaacatgtgcgccgtgattgacgggaacagcaccaccttcgagcaccagcagccgttgcaggaccggatgttcaaatttgaactcacccgccgtctggagcacgactttggcaaggtgacgaagcaggaagtcaaagagttcttccgctgggccagtgatcacgtgaccgaggtggcgcatgagttctacgtcagaaagggcggagccagcaaaagacccgcccccgatgacgcggatataagcgagcccaagcgggcctgcccctcagtcgcggatccatcgacgtcagacgcggaaggagctccggtggactttgccgacaggtaccaaaacaaatgttctcgtcacgcgggcatgattcagatgctgtttccctgcaaaacgtgcgagagaatgaatcagaatttcaacatttgcttcacacacggggtcagagactgtttagagtgtttccccggcgtgtcagaatctcaaccggtcgtcagaaaaaagacgtatcggaaactctgcgcgattcatcatctgctggggcgggcgcccgagattgcttgctcggcctgcgacctggtcaacgtggacctggacgactgcgtttctgagcaataaatgacttaaaccaggtatggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcatttgggggcaacctcgggcgagcagtcttccaggccaagaagcgggttctcgaacctctcggtctggttgaggaaggcgctaagacggctcctgcaaagaagagaccggtagagccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcattaccaccagcacccgaacctgggccctgcccacctacaacaaccacctctacaagcaaatctccagtgaaactgcaggtagtaccaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattcctcagtacggctacctgactctcaacaatggcagtcagtctgtgggacgttcctccttctactgcctggagtacttcccctctcagatgctgagaacgggcaacaactttgagttcagctacagcttcgaggacgtgcctttccacagcagctacgcacacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctcctcagatcctgatcaagaacactcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacctccaactttgaaaagcagactggtgtggactttgccgttgacagccagggtgtttactctgagcctcgccctattggcactcgttacctcacccgtaatctgtaattgcatgttaatcaataaaccggttgattcgtttcagttgaactttggtctcctgtgcttcttatcttatcggtttccatagcaactggttacacattaactgcttgggtgcgcttcacgataagaacactgacgtcaccgcggtacccctagtgatggagttggccactccctctatgcgcgctcgctcgctcggtggggcctgcggaccaaaggtccgcagacggcagagctctgctctgccggccccaccgagcgagcgagcgcgcatagagggagtggccaa

SEQ ID NO: 17 - protein, 737 aa, Adeno-associated virus
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 18 - DNA, 4393 bp, Adeno-associated virus
cagagagggagtggccaactccatcactaggggtagcgcgaagcgcctcccacgctgccgcgtcagcgctgacgtaaattacgtcataggggagtggtcctgtattagctgtcacgtgagtgcttttgcggcattttgcgacaccacgtggccatttgaggtatatatggccgagtgagcgagcaggatctccattttgaccgcgaaatttgaacgagcagcagccatgccgggcttctacgagatcgtgatcaaggtgccgagcgacctggacgagcacctgccgggcatttctgactcgtttgtgaactgggtggccgagaaggaatgggagctgcccccggattctgacatggatcggaatctgatcgagcaggcacccctgaccgtggccgagaagctgcagcgcgacttcctggtccaatggcgccgcgtgagtaaggccccggaggccctcttctttgttcagttcgagaagggcgagagctactttcacctgcacgttctggtcgagaccacgggggtcaagtccatggtgctaggccgcttcctgagtcagattcgggaaaagcttggtccagaccatctacccgcggggtcgagccccaccttgcccaactggttcgcggtgaccaaagacgcggtaatggcgccggcgggggggaacaaggtggtggacgagtgctacatccccaactacctcctgcccaagactcagcccgagctgcagtgggcgtggactaacatggaggagtatataagcgcgtgcttgaacctggccgagcgcaaacggctcgtggcgcagcacctgacccacgtcagccagacgcaggagcagaacaaggagaatctgaaccccaattctgacgcgcccgtgatcaggtcaaaaacctccgcgcgctatatggagctggtcgggtggctggtggaccggggcatcacctccgagaagcagtggatccaggaggaccaggcctcgtacatctccttcaacgccgcctccaactcgcggtcccagatcaaggccgcgctggacaatgccggcaagatcatggcgctgaccaaatccgcgcccgactacctggtggggccctcgctgcccgcggacattacccagaaccgcatctaccgcatcctcgctctcaacggctacgaccctgcctacgccggctccgtctttctcggctgggctcagaaaaagttcgggaaacgcaacaccatctggctgtttggacccgccaccaccggcaagaccaacattgcggaagccatcgcccacgccgtgcccttctacggctgcgtcaactggaccaatgagaactttcccttcaatgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccacccccgtgatcgtcacctccaacaccaacatgtgcgccgtgattgacgggaacagcaccaccttcgagcaccagcagcctctccaggaccggatgtttaagttcgaactcacccgccgtctggagcacgactttggcaaggtgacaaagcaggaagtcaaagagttcttccgctgggccagtgatcacgtgaccgaggtggcgcatgagttttacgtcagaaagggcggagccagcaaaagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaaaacgtgcgagagaatgaatcagaatttcaacatttgcttcacacacggggtcagagactgctcagagtgtttccccggcgtgtcagaatctcaaccggtcgtcagaaagaggacgtatcggaaactctgtgcgattcatcatctgctggggcgggctcccgagattgcttgctcggcctgcgatctggtcaacgtggacctggatgactgtgtttctgagcaataaatgacttaaaccaggtatggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaattgcctgttaatcaataaaccggttgattcgtttcagttgaactttggtctctgcg

SEQ ID NO: 19 - DNA, 2217 bp, Adeno-associated virus
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 20 - protein, 738 aa, Adeno-associated virus
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 21 - DNA, 2211 bp, Adeno-associated virus
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 22 - protein, 736 aa, Adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 23 - DNA, 2232 bp, Artificial Sequence
  Description of Artificial Sequence Synthetic polynucleotide
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctagaactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggtaccggcagagacaacgtggatgcggacaaagtcatgataaccaacgaagaagaaattaaaactactaacccggtagcaacggagtcctatggacaagtggccacaaaccaccagagtgcccaaactttggcggtgccttttaaggcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 24 - protein, 743 aa, Artificial Sequence
  Description of Artificial Sequence Synthetic polypeptide
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 25 - protein, 736 aa, Artificial Sequence
  Description of Artificial Sequence Synthetic polypeptide
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTIQSSQTPRQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 26 - protein, 743 aa, Artificial Sequence
  Description of Artificial Sequence Synthetic polypeptide
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTIILGTGTSQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTRTNPEAAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 27 - protein, 743 aa, Artificial Sequence
  Description of Artificial Sequence Synthetic polypeptide
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTIILGTGTSQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQNGGTSSSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 28 - protein, 743 aa, Artificial Sequence
  Description of Artificial Sequence Synthetic polypeptide
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 29 - protein, 743 aa, Artificial Sequence
  Description of Artificial Sequence Synthetic polypeptide
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQQAVRTSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 30 - protein, 736 aa, Artificial Sequence
  Description of Artificial Sequence Synthetic polypeptide
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 31 - DNA, 3129 bp, Adeno-associated virus
gaattcgccctttctacggctgcgtcaactggaccaatgagaactttcccttcaacgattgcgtcgacaagatggtgatctggtgggaggagggcaagatgacggccaaggtcgtggagtccgccaaggccattctcggcggcagcaaagtgcgcgtggaccaaaagtgcaagtcgtccgcccagatcgaccccacccccgtgatcgtcacctccaacaccaacatgtgcgccgtgattgacgggaacagcaccaccttcgagcaccagcagccgttgcaggaccggatgttcaagtttgaactcacccgccgtctggagcacgactttggcaaggtgacaaagcaggaagtcagagagttcttccgctgggcgcaggatcacgtgaccgaggtggcgcacgagttctacgtcagaaagggtggagccaacaagagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaaaacatgcgagagaatgaatcagaatttcaacatttgcttcacgcacgggaccagagactgttcagaatgtttccccggcgtgtcagaatctcaaccggtcgtcagaaaaaagacgtatcggaaactctgtgcgattcatcatctgctgggggcgggcacccgagattgcttgctcggcctgcgatctggtcaacgtggacctagatgactgtgtttctgagcaataaatgacttaaaccaggtatggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgcgaaaaagagactcaactttgggcagactggcgactcagagtcagtgcccgaccctcaaccaatcggagaaccccccgcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctccccacctacaacaaccacctctacaagcaaatctccaacgggacttcgggaggaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacgccgctcctattgtaggggccgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagatggcacttattctgagcctcgccccatcggcacccgttacctcacccgtaatctgtaattgcttgttaatcaataaaccggttgattcgtttcagttgaactttggtctctgcgaagggcgaattc

SEQ ID NO: 32 - protein, 738 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 33 - DNA, 2215 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcactctcctgtggagccagactcctcctcgggaaccggaaaggcgggccagcagcctgcaagaaaaagattgaattttggtcagactggagacgcagactcagtcccagaccctcaaccaatcggagaacctcccgcagccccctcaggtgtgggatctcttacaatggctgcaggcggtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaagcaaatctccaacagcacatctggaggatcttcaaatgacaacgcctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacgacaaatacgcagactctgggcttcagccaaggtgggcctaatacaatggccaatcaggcaaagaactggctgccaggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagtttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctccgcctcagatcctgatcaagaacacgcctgtacctgcggatcctccgaccaccttcaaccagtcaaagctgaactctttcatcacccagtattctactggccaagtcagcgtggagatcgagtgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 34 - protein, 737 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLKTGNNFQFTYTFEDVPFHSSYAHSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 35 - protein, 737 aa, Artificial Sequence
  Description of Artificial Sequence Synthetic polypeptide
  R587Q and R590T mutations of SEQ ID NO 1 of 7588772
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLKTGNNFQFTYTFEDVPFHSSYAHSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQQGNTQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 36 - protein, 737 aa, Artificial Sequence
  Description of Artificial Sequence Synthetic polypeptide
  K406R, R587Q and R590T mutations of SEQ ID NO 1 of 7588772
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQQGNTQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 37 - DNA, 2217 bp, Adeno-associated virus
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagagaccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaagctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa

SEQ ID NO: 38 - protein, 738 aa, Adeno-associated virus
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 39 - protein, 738 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGESESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQANTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 40 - protein, 738 aa, Adeno-associated virus; identical to SEQ ID NO: 32

SEQ ID NO: 41 - DNA, 2301 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcgtcgacataacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttgaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtgtgctgggcggggggcggcgggccctcccgcagaacaccatgcgctccacggaacctgctagctccatgcttgctgtggccactgagaagtaagcatggagctagcaggttctgaggagcgccttgacagcagccatgggagggccgccccctacctcagtgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcgactagatttaaataattcgcccttgggcctaggcaattggatccgccggcagagaaaacatcccagggatttacagatcacatgcaggcagggaccagctcaacccttctttaatgtcatccagggagggggccagggatggaggggaggggttgaggagcgagaggcagttatttttgggtgggattcaccacttttcccatgaagaggggagacttggtattttgttcaatcattaagaagacaaagggtttgttgaacttgacctcgggggggatagacatgggtatggcctctaaaaacatggccccagcagcttcagtccctttctcgtcgatggtcagcacagccttatgcacggcctggaggggagagaagcagagacacgttgtaaggctgatcccaggcctcgagcaaggctcacgtggacacctcccaggaagcgctcactccccctggacggccctggccctgcacatcctctccctccctgtcacataggccttgctcctcctcaaggctttggctgatggggctggctcccctctgtccatcttcctgacaagcgcctctccccctgctcaggtgcacccacaactcagaacagggaagagcatcgtcactccacgtctgcctccagggctctctcctttctagtacacggcttgaagctccttgaggacacggaccctggcagtgaccttcacagtgcccagaccatttaaatatgtcgtgcatcgatgctacgtagataagtagcatggcgggttaatcattaactacagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 42 - DNA, 4608 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcgtcgacataacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttgaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtgtgctgggcggggggcggcgggccctcccgcagaacaccatgcgctccacggaacctgctagctccatgcttgctgtggccactgagaagtaagcatggagctagcaggttctgaggagcgccttgacagcagccatgggagggccgccccctacctcagtgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcgactagatttaaataattcgcccttgggcctaggcaattggatccgccggcagagaaaacatcccagggatttacagatcacatgcaggcagggaccagctcaacccttctttaatgtcatccagggagggggccagggatggaggggaggggttgaggagcgagaggcagttatttttgggtgggattcaccacttttcccatgaagaggggagacttggtattttgttcaatcattaagaagacaaagggtttgttgaacttgacctcgggggggatagacatgggtatggcctctaaaaacatggccccagcagcttcagtccctttctcgtcgatggtcagcacagccttatgcacggcctggaggggagagaagcagagacacgttgtaaggctgatcccaggcctcgagcaaggctcacgtggacacctcccaggaagcgctcactccccctggacggccctggccctgcacatcctctccctccctgtcacataggccttgctcctcctcaaggctttggctgatggggctggctcccctctgtccatcttcctgacaagcgcctctccccctgctcaggtgcacccacaactcagaacagggaagagcatcgtcactccacgtctgcctccagggctctctcctttctagtacacggcttgaagctccttgaggacacggaccctggcagtgaccttcacagtgcccagaccccaagataatgcagccattcatggaactgcaggttgttcattggtcgcctttagttttccaaaataagtgtcactttagctgaaatcattcattaattcagacaccaaatctcacagatcgaaggagtcagaaattcctttgaaacaacttagcccaaacctttctgtgtcagtatggataaatcaaggcccaatgtctagaaggtcttgggcaaagttgaaattcagggtcagtgacacaacctcaagggaggccccgaaagtgccagctgcacagcagcccctgcctggctttgctgtttgcccaccgtcccgtgtcagtgaatcacgggcatcttcaggagctcagcctgggtcttcatttgtttccctcggccccttcctcagcctcaggacagtgctgcagcccccacacattcttccctacagataccatggtgcaacaaggtcgtcagggtgatctcaccttggagagcttcaggggtgcctcctctgtgaccccggagaggtcagccccattgctgaagaccttagtgatgcccagttgacccaggacgctcttcagatcataggttccagtaatggacagtttgggtaaatgtaagctggcagacctgtcgtgcagaaaagaaattcaaggcatggcacagcattcctcttgttcttctgggacccaccacagtgcaagtgttttcttttctgattatttctgccacttactcctgtgtcctccacccacactaagatgggaactcggctttggtttgttctacttttagctcttctacattgagtcaaagaatgttaacatcgaatgaatcacaaaagcttgaaatgccacctcctctgatattctaggtgtcctggaagcctgtctcatcttgccctgtagtgttgggtcacctggcccccagcctgtaacatccccagggccctacacccagagaaacacggggctggtggcagtgcccagtgacaaccgtttagtggataagagaagagtgaccacaccaggctgagtgctcctctctggttttccatggggagacaatgccaccctgagcagggtctggtgtgagcggcagctggctctgggctctctgatccgttaccctctcagcctctttgttctttctcaacccctggagcagagacctcaggaggtgctggcatggaacagagaaattccagcctcgattcctattatgaacccgacaccttttgtattttcatcttggttttacagtgtacaaaacgaactagatcagcagggcatgggcataatcacgaatgcacacacatacactaatgtgtggctcatgtttaagtatcacttactacaggacacccaatctaacagcaccgataaagtgacagagaaacgcaagccttctgcgaacatggcctggctgttccaattccgaaccttgcttttctgggccttgccacacaggctcttcccccgtccccccagggacattctacccttgaactccacactccactgctgcctttgccaggaagcccatctgttcctttttggttctgccagaacgtgtggtggtgctgctgtccctgccttgggcactggatattgggaagggacagtgtccacactggagtgggaagttcccagggacgagacctttacctcctcaccctgggtactgttctcctcatggagcatggacggcgctgcctgaactcagtggtggcctcattctggaagccaagtttatacagagtagcagtgacccagggatgtggggttcaccctcctcagccctctggccagtcctgatgggcctcagtcccaacatggctaagaggtgtgggcagcttcttggtcaccctcaggttggggaatcaccttctgtcttcattttccaggaacttggtgatgatatcgtgggtgagttcatttaccaggtgctgtagtttcccctcatcaggcaggaagaagatggcggtggcattgcccaggtatttcatcagcagcacccagctggacagcttcttacagtgctggatgttaaacatgcctaaacgcttcatcataggcaccttcacggtggtcacctggtccacgtggaagtcctcttcctcggtgtccttgacttcaaagggtctctcccatttgcctggagagaggggaaggtgggcatcaccaggggtgagtgaaggtttggaagagtgtagcagaataagaaaccatgagtcccctccctgagaagccctgagcccccttgacgacacacatccctcgaggctcagcttcatcatctgtaaaaggtgctgaaactgaccatttaaatatgtcgtgcatcgatgctacgtagataagtagcatggcgggttaatcattaactacagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 43 - DNA, 2296 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcgtcgacataacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttgaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtgtgctgggcggggggcggcgggccctcccgcagaacaccatgcgctcttcggaagcaattcagtctcgttgtacctgtgatttggtacaacgagactgaattgcttctgaggagcgccttgacagcagccatgggagggccgccccctacctcagtgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcgactagatttaaataattcgcccttgggcctaggcaattggatccgccggcagagaaaacatcccagggatttacagatcacatgcaggcagggaccagctcaacccttctttaatgtcatccagggagggggccagggatggaggggaggggttgaggagcgagaggcagttatttttgggtgggattcaccacttttcccatgaagaggggagacttggtattttgttcaatcattaagaagacaaagggtttgttgaacttgacctcgggggggatagacatgggtatggcctctaaaaacatggccccagcagcttcagtccctttctcgtcgatggtcagcacagccttatgcacggcctggaggggagagaagcagagacacgttgtaaggctgatcccaggcctcgagcaaggctcacgtggacacctcccaggaagcgctcactccccctggacggccctggccctgcacatcctctccctccctgtcacataggccttgctcctcctcaaggctttggctgatggggctggctcccctctgtccatcttcctgacaagcgcctctccccctgctcaggtgcacccacaactcagaacagggaagagcatcgtcactccacgtctgcctccagggctctctcctttctagtacacggcttgaagctccttgaggacacggaccctggcagtgaccttcacagtgcccagaccatttaaatatgtcgtgcatcgatgctacgtagataagtagcatggcgggttaatcattaactacagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 44 - DNA, 2301 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcgtcgacataacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttgaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtgtgctgggcggggggcggcgggccctcccgcagaacaccatgcgctcctcggaataagcatggagctagcaggttgtggccactgagaaacctgctagctccatgcttgttctgcggagcgccttgacagcagccatgggagggccgccccctacctcagtgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcgactagatttaaataattcgcccttgggcctaggcaattggatccgccggcagagaaaacatcccagggatttacagatcacatgcaggcagggaccagctcaacccttctttaatgtcatccagggagggggccagggatggaggggaggggttgaggagcgagaggcagttatttttgggtgggattcaccacttttcccatgaagaggggagacttggtattttgttcaatcattaagaagacaaagggtttgttgaacttgacctcgggggggatagacatgggtatggcctctaaaaacatggccccagcagcttcagtccctttctcgtcgatggtcagcacagccttatgcacggcctggaggggagagaagcagagacacgttgtaaggctgatcccaggcctcgagcaaggctcacgtggacacctcccaggaagcgctcactccccctggacggccctggccctgcacatcctctccctccctgtcacataggccttgctcctcctcaaggctttggctgatggggctggctcccctctgtccatcttcctgacaagcgcctctccccctgctcaggtgcacccacaactcagaacagggaagagcatcgtcactccacgtctgcctccagggctctctcctttctagtacacggcttgaagctccttgaggacacggaccctggcagtgaccttcacagtgcccagaccatttaaatatgtcgtgcatcgatgctacgtagataagtagcatggcgggttaatcattaactacagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 45 - DNA, 4606 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcgtcgacataacgcgtaattcgcccttgggcctaggcaattggatccgccggcagagaaaacatcccagggatttacagatcacatgcaggcagggaccagctcaacccttctttaatgtcatccagggagggggccagggatggaggggaggggttgaggagcgagaggcagttatttttgggtgggattcaccacttttcccatgaagaggggagacttggtattttgttcaatcattaagaagacaaagggtttgttgaacttgacctcgggggggatagacatgggtatggcctctaaaaacatggccccagcagcttcagtccctttctcgtcgatggtcagcacagccttatgcacggcctggaggggagagaagcagagacacgttgtaaggctgatcccaggcctcgagcaaggctcacgtggacacctcccaggaagcgctcactccccctggacggccctggccctgcacatcctctccctccctgtcacataggccttgctcctcctcaaggctttggctgatggggctggctcccctctgtccatcttcctgacaagcgcctctccccctgctcaggtgcacccacaactcagaacagggaagagcatcgtcactccacgtctgcctccagggctctctcctttctagtacacggcttgaagctccttgaggacacggaccctggcagtgaccttcacagtgcccagaccccaagataatgcagccattcatggaactgcaggttgttcattggtcgcctttagttttccaaaataagtgtcactttagctgaaatcattcattaattcagacaccaaatctcacagatcgaaggagtcagaaattcctttgaaacaacttagcccaaacctttctgtgtcagtatggataaatcaaggcccaatgtctagaaggtcttgggcaaagttgaaattcagggtcagtgacacaacctcaagggaggccccgaaagtgccagctgcacagcagcccctgcctggctttgctgtttgcccaccgtcccgtgtcagtgaatcacgggcatcttcaggagctcagcctgggtcttcatttgtttccctcggccccttcctcagcctcaggacagtgctgcagcccccacacattcttccctacagataccatggtgcaacaaggtcgtcagggtgatctcaccttggagagcttcaggggtgcctcctctgtgaccccggagaggtcagccccattgctgaagaccttagtgatgcccagttgacccaggacgctcttcagatcataggttccagtaatggacagtttgggtaaatgtaagctggcagacctgtcgtgcagaaaagaaattcaaggcatggcacagcattcctcttgttcttctgggacccaccacagtgcaagtgttttcttttctgattatttctgccacttactcctgtgtcctccacccacactaagatgggaactcggctttggtttgttctacttttagctcttctacattgagtcaaagaatgttaacatcgaatgaatcacaaaagcttgaaatgccacctcctctgatattctaggtgtcctggaagcctgtctcatcttgccctgtagtgttgggtcacctggcccccagcctgtaacatccccagggccctacacccagagaaacacggggctggtggcagtgcccagtgacaaccgtttagtggataagagaagagtgaccacaccaggctgagtgctcctctctggttttccatggggagacaatgccaccctgagcagggtctggtgtgagcggcagctggctctgggctctctgatccgttaccctctcagcctctttgttctttctcaacccctggagcagagacctcaggaggtgctggcatggaacagagaaattccagcctcgattcctattatgaacccgacaccttttgtattttcatcttggttttacagtgtacaaaacgaactagatcagcagggcatgggcataatcacgaatgcacacacatacactaatgtgtggctcatgtttaagtatcacttactacaggacacccaatctaacagcaccgataaagtgacagagaaacgcaagccttctgcgaacatggcctggctgttccaattccgaaccttgcttttctgggccttgccacacaggctcttcccccgtccccccagggacattctacccttgaactccacactccactgctgcctttgccaggaagcccatctgttcctttttggttctgccagaacgtgtggtggtgctgctgtccctgccttgggcactggatattgggaagggacagtgtccacactggagtgggaagttcccagggacgagacctttacctcctcaccctgggtactgttctcctcatggagcatggacggcgctgcctgaactcagtggtggcctcattctggaagccaagtttatacagagtagcagtgacccagggatgtggggttcaccctcctcagccctctggccagtcctgatgggcctcagtcccaacatggctaagaggtgtgggcagcttcttggtcaccctcaggttggggaatcaccttctgtcttcattttccaggaacttggtgatgatatcgtgggtgagttcatttaccaggtgctgtagtttcccctcatcaggcaggaagaagatggcggtggcattgcccaggtatttcatcagcagcacccagctggacagcttcttacagtgctggatgttaaacatgcctaaacgcttcatcataggcaccttcacggtggtcacctggtccacgtggaagtcctcttcctcggtgtccttgacttcaaagggtctctcccatttgcctggagagaggggaaggtgggcatcaccaggggtgagtgaaggtttggaagagtgtagcagaataagaaaccatgagtcccctccctgagaagccctgagcccccttgacgacacacatccctcgaggctcagcttcatcatctgtaaaaggtgctgaaactgaccacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttgaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtgtgctgggcggggggcggcgggccctcccgcagaacaccatgcgctccacggaacctgctagctccatgcttgctgtggccactgagaagtaagcatggagctagcaggttctgaggagcgccttgacagcagccatgggagggccgccccctacctcagtgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcgactagatttaaatatgtcgtgcatcgatgctacgtagataagtagcatggcgggttaatcattaactacagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 46 - DNA, 2299 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcgtcgacataacgcgtaattcgcccttgggcctaggcaattggatccgccggcagagaaaacatcccagggatttacagatcacatgcaggcagggaccagctcaacccttctttaatgtcatccagggagggggccagggatggaggggaggggttgaggagcgagaggcagttatttttgggtgggattcaccacttttcccatgaagaggggagacttggtattttgttcaatcattaagaagacaaagggtttgttgaacttgacctcgggggggatagacatgggtatggcctctaaaaacatggccccagcagcttcagtccctttctcgtcgatggtcagcacagccttatgcacggcctggaggggagagaagcagagacacgttgtaaggctgatcccaggcctcgagcaaggctcacgtggacacctcccaggaagcgctcactccccctggacggccctggccctgcacatcctctccctccctgtcacataggccttgctcctcctcaaggctttggctgatggggctggctcccctctgtccatcttcctgacaagcgcctctccccctgctcaggtgcacccacaactcagaacagggaagagcatcgtcactccacgtctgcctccagggctctctcctttctagtacacggcttgaagctccttgaggacacggaccctggcagtgaccttcacagtgcccagaccacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttgaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtgtgctgggcggggggcggcgggccctcccgcagaacaccatgcgctccacggaacctgctagctccatgcttgctgtggccactgagaagtaagcatggagctagcaggttctgaggagcgccttgacagcagccatgggagggccgccccctacctcagtgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcgactagatttaaatatgtcgtgcatcgatgctacgtagataagtagcatggcgggttaatcattaactacagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 47 - DNA, 2294 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcgtcgacataacgcgtaattcgcccttgggcctaggcaattggatccgccggcagagaaaacatcccagggatttacagatcacatgcaggcagggaccagctcaacccttctttaatgtcatccagggagggggccagggatggaggggaggggttgaggagcgagaggcagttatttttgggtgggattcaccacttttcccatgaagaggggagacttggtattttgttcaatcattaagaagacaaagggtttgttgaacttgacctcgggggggatagacatgggtatggcctctaaaaacatggccccagcagcttcagtccctttctcgtcgatggtcagcacagccttatgcacggcctggaggggagagaagcagagacacgttgtaaggctgatcccaggcctcgagcaaggctcacgtggacacctcccaggaagcgctcactccccctggacggccctggccctgcacatcctctccctccctgtcacataggccttgctcctcctcaaggctttggctgatggggctggctcccctctgtccatcttcctgacaagcgcctctccccctgctcaggtgcacccacaactcagaacagggaagagcatcgtcactccacgtctgcctccagggctctctcctttctagtacacggcttgaagctccttgaggacacggaccctggcagtgaccttcacagtgcccagaccacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttgaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtgtgctgggcggggggcggcgggccctcccgcagaacaccatgcgctcttcggaagcaattcagtctcgttgtacctgtgatttggtacaacgagactgaattgcttctgaggagcgccttgacagcagccatgggagggccgccccctacctcagtgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcgactagatttaaatatgtcgtgcatcgatgctacgtagataagtagcatggcgggttaatcattaactacagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 48 - DNA, 2299 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcgtcgacataacgcgtaattcgcccttgggcctaggcaattggatccgccggcagagaaaacatcccagggatttacagatcacatgcaggcagggaccagctcaacccttctttaatgtcatccagggagggggccagggatggaggggaggggttgaggagcgagaggcagttatttttgggtgggattcaccacttttcccatgaagaggggagacttggtattttgttcaatcattaagaagacaaagggtttgttgaacttgacctcgggggggatagacatgggtatggcctctaaaaacatggccccagcagcttcagtccctttctcgtcgatggtcagcacagccttatgcacggcctggaggggagagaagcagagacacgttgtaaggctgatcccaggcctcgagcaaggctcacgtggacacctcccaggaagcgctcactccccctggacggccctggccctgcacatcctctccctccctgtcacataggccttgctcctcctcaaggctttggctgatggggctggctcccctctgtccatcttcctgacaagcgcctctccccctgctcaggtgcacccacaactcagaacagggaagagcatcgtcactccacgtctgcctccagggctctctcctttctagtacacggcttgaagctccttgaggacacggaccctggcagtgaccttcacagtgcccagaccacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttgaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtgtgctgggcggggggcggcgggccctcccgcagaacaccatgcgctcctcggaataagcatggagctagcaggttgtggccactgagaaacctgctagctccatgcttgttctgcggagcgccttgacagcagccatgggagggccgccccctacctcagtgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcgactagatttaaatatgtcgtgcatcgatgctacgtagataagtagcatggcgggttaatcattaactacagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 49 - DNA, 4618 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcgtcgacataacgcgtaattcgcccttgggcctaggcaattggatccgccggcagagaaaacatcccagggatttacagatcacatgcaggcagggaccagctcaacccttctttaatgtcatccagggagggggccagggatggaggggaggggttgaggagcgagaggcagttatttttgggtgggattcaccacttttcccatgaagaggggagacttggtattttgttcaatcattaagaagacaaagggtttgttgaacttgacctcgggggggatagacatgggtatggcctctaaaaacatggccccagcagcttcagtccctttctcgtcgatggtcagcacagccttatgcacggcctggaggggagagaagcagagacacgttgtaaggctgatcccaggcctcgagcaaggctcacgtggacacctcccaggaagcgctcactccccctggacggccctggccctgcacatcctctccctccctgtcacataggccttgctcctcctcaaggctttggctgatggggctggctcccctctgtccatcttcctgacaagcgcctctccccctgctcaggtgcacccacaactcagaacagggaagagcatcgtcactccacgtctgcctccagggctctctcctttctagtacacggcttgaagctccttgaggacacggaccctggcagtgaccttcacagtgcccagaccccaagataatgcagccattcatggaactgcagttgttcattggtcgcctttagttttccaaaataagtgtcatctttagctgaaatcattcattaattcagacaccaaatctcacagatcgaaggagtcagaaattcctttgaaacaacttagcccaaacctttctgtgtcagtatggataaatcaaggcccaatgtctagaaggtcttgggcaaagttgaaattcagggtcagtgacacaacctcaagggaggccccgaaagtgccagctgcacagcagtcccctgcctggctttgctgtttgaccacgtcccgtgtcagtgaatcacgggcatcttcaggagctcagcctgggtcttcatttgtttccctcggccccttcctcagcctcaggacagagctgcagcccccacacattcttccctacagataccatggtgcaacaaggtcgtcagggtgatctcaccttggagagcttcaggggtgcctcctctgtgaccccggagaggtcagccccattgctgaagaccttagtgatgcccagttgacccaggacgctcttcagatcataggttccagtaatggacagtttgggtaaatgtaagctggcagacctgtcgtgcagaaaagaaattcaaggcatggcacagcattcctcttgttcttctgggacccaccacagtgcaagtgttttcttttctgattatttctgccacttactcctgtgtcctccacccacactaagatgggaactcggctttggtttgttctacttttagctcttctacattgagtcaaagacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttgaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtgtgctgggcggggggcggcgggccctcccgcagaacaccatgcgctccacggaacctgctagctccatgcttgctgtggccactgagaagtaagcatggagctagcaggttctgaggagcgccttgacagcagccatgggagggccgccccctacctcagtgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcgactagatttaaataatgttaacatcgaatgaatcacaaaagcttgaaatgccacctcctctgatattctaggtgtcctggaagcctgtctcatcttgccctgtagtgttgggtcacctggcccccagcctgtaacatccccagggccctacacccagagaaacacggggctggtggcagtgcccagtgacaaccgtttagtggataagagaagagtgaccacaccaggctgagtgctcctctctggttttccatggggagacaatgccaccctgagcagggtctggtgtgagcgggcagcttggctctgggctctctgatccgttaccctctcagcctctttgttctttctcaacccctggagcagagacctcaggaggtgctggcatggaacagagaaattccagcctcgattcctattatgaacccgacaccttttgtattttcatcttggttttacagtgtacaaaacgaactagatcagcagggcatgggcataatcacgaatgcacacacatacactaatgtgtggctcatgtttaagtatcacttactacaggacacccaatctaacagcaccgataaagtgacagagaaacgcaagccttctgcgaacatggcctggctgttccaattccgaaccttgcttttctgggccttgccacacaggctcttcccccgtccccccagggacattctacccttgaactccacactccactgctgcctttgccaggaagcccatctgttcctttttggttctgccagaacgtgtggtggtgctgctgtccctgccctgggcactggatattgggaagggacagtgtccacactggagtgggaagtccccagggacgagacctttacctcctcacccctgggtactgtcctcctcatggagcatggatggcgctgcctgaactcagtggtggcctcattctggaagccaagtttatacagagtagcagtgacccagggatgtggggttcaccctcctcagccctctggccagtcctgatgggcctcagtcccaacatggctaagaggtgtgggcagcttcttggtcaccctcaggttggggaatcaccttctgtcttcattttccaggaacttggtgatgatatcgtgggtgagttcattttccaggtgctgtagtttcccctcatcaggcaggaagaagatggcggtggcattgcccaggtatttcatcagcagcacccagctggacagcttcttacagtgctggatgttaaacatgcctaaacgcttcatcataggcaccttcacggtggtcacctggtccacgtggaagtcctcttcctcggtgtccttgacttcaaagggtctctcccatttgcctggagagaggggaaggtgggcatcaccaggggtgagtgaaggtttggaagagtgtagcagaataaagaaaccatgagtcccctccctgagaagccctgagcccccttgacgacacacatccctcgaggctcagcttcatcatctgtaaaaggtgctgaaactgaccatttaaatatgtcgtgcatcgatgctacgtagataagtagcatggcgggttaatcattaactacagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 50 - DNA, 2307 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcgtcgacataacgcgtaattcgcccttgggcctaggcaattggatccgccggcagagaaaacatcccagggatttacagatcacatgcaggcagggaccagctcaacccttctttaatgtcatccagggagggggccagggatggaggggaggggttgaggagcgagaggcagttatttttgggtgggattcaccacttttcccatgaagaggggagacttggtattttgttcaatcattaagaagacaaagggtttgttgaacttgacctcgggggggatagacatgggtatggcctctaaaaacatggccccagcagcttcagtccctttctcgtcgatggtcagcacagccttatgcacggcctggagggacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttgaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtgtgctgggcggggggcggcgggccctcccgcagaacaccatgcgctccacggaacctgctagctccatgcttgctgtggccactgagaagtaagcatggagctagcaggttctgaggagcgccttgacagcagccatgggagggccgccccctacctcagtgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcgactagatttaaatgagagaagcagagacacgttgtaaggctgatcccaggcctcgagcaaggctcacgtggacacctcccaggaagcgctcactccccctggacggccctggccctgcacatcctctccctccctgtcacataggccttgctcctcctcaaggctttggctgatggggctggctcccctctgtccatcttcctgacaagcgcctctccccctgctcaggtgcacccacaactcagaacagggaagagcatcgtcactccacgtctgcctccagggctctctcctttctagtacacggcttgaagctccttgaggacacggaccctggcagtgaccttcacagtgcccagaccatttaaatatgtcgtgcatcgatgctacgtagataagtagcatggcgggttaatcattaactacagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 51 - DNA, 2302 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcgtcgacataacgcgtaattcgcccttgggcctaggcaattggatccgccggcagagaaaacatcccagggatttacagatcacatgcaggcagggaccagctcaacccttctttaatgtcatccagggagggggccagggatggaggggaggggttgaggagcgagaggcagttatttttgggtgggattcaccacttttcccatgaagaggggagacttggtattttgttcaatcattaagaagacaaagggtttgttgaacttgacctcgggggggatagacatgggtatggcctctaaaaacatggccccagcagcttcagtccctttctcgtcgatggtcagcacagccttatgcacggcctggagggacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttgaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtgtgctgggcggggggcggcgggccctcccgcagaacaccatgcgctcttcggaagcaattcagtctcgttgtacctgtgatttggtacaacgagactgaattgcttctgaggagcgccttgacagcagccatgggagggccgccccctacctcagtgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcgactagatttaaatgagagaagcagagacacgttgtaaggctgatcccaggcctcgagcaaggctcacgtggacacctcccaggaagcgctcactccccctggacggccctggccctgcacatcctctccctccctgtcacataggccttgctcctcctcaaggctttggctgatggggctggctcccctctgtccatcttcctgacaagcgcctctccccctgctcaggtgcacccacaactcagaacagggaagagcatcgtcactccacgtctgcctccagggctctctcctttctagtacacggcttgaagctccttgaggacacggaccctggcagtgaccttcacagtgcccagaccatttaaatatgtcgtgcatcgatgctacgtagataagtagcatggcgggttaatcattaactacagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 52 - DNA, 2307 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcgtcgacataacgcgtaattcgcccttgggcctaggcaattggatccgccggcagagaaaacatcccagggatttacagatcacatgcaggcagggaccagctcaacccttctttaatgtcatccagggagggggccagggatggaggggaggggttgaggagcgagaggcagttatttttgggtgggattcaccacttttcccatgaagaggggagacttggtattttgttcaatcattaagaagacaaagggtttgttgaacttgacctcgggggggatagacatgggtatggcctctaaaaacatggccccagcagcttcagtccctttctcgtcgatggtcagcacagccttatgcacggcctggagggacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttgaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtgtgctgggcggggggcggcgggccctcccgcagaacaccatgcgctcctcggaataagcatggagctagcaggttgtggccactgagaaacctgctagctccatgcttgttctgcggagcgccttgacagcagccatgggagggccgccccctacctcagtgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcgactagatttaaatgagagaagcagagacacgttgtaaggctgatcccaggcctcgagcaaggctcacgtggacacctcccaggaagcgctcactccccctggacggccctggccctgcacatcctctccctccctgtcacataggccttgctcctcctcaaggctttggctgatggggctggctcccctctgtccatcttcctgacaagcgcctctccccctgctcaggtgcacccacaactcagaacagggaagagcatcgtcactccacgtctgcctccagggctctctcctttctagtacacggcttgaagctccttgaggacacggaccctggcagtgaccttcacagtgcccagaccatttaaatatgtcgtgcatcgatgctacgtagataagtagcatggcgggttaatcattaactacagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 53 - DNA, 1587 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcgtcgacataacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttgaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtgtgctgggcggggggcggcgggccctcccgcagaacaccatgcgctccacggaacctgctagctccatgcttgctgtggccactgagaagtaagcatggagctagcaggttctgaggagcgccttgacagcagccatgggagggccgccccctacctcagtgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcgactagatttaaatatgtcgtgcatcgatgctacgtagataagtagcatggcgggttaatcattaactacagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 54 - DNA, 1582 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcgtcgacataacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttgaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtgtgctgggcggggggcggcgggccctcccgcagaacaccatgcgctcttcggaagcaattcagtctcgttgtgcctgtgacctggtacaacgagactgaattgcttctgaggagcgccttgacagcagccatgggagggccgccccctacctcagtgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcgactagatttaaatatgtcgtgcatcgatgctacgtagataagtagcatggcgggttaatcattaactacagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 55 - DNA, 1584 bp, Artificial Sequence
  Description of Artificial Sequence Synthetic polynucleotide
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcaattcacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttgaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtgaagcaaagaaggggcagagggagcccgtgagctgagtgggccagggactgggagaaggagtgaggaggcagggccggcatgcctctgctgctggccagacctgctagctccatgcttcccgtctgcacctgtcactagtaagcatggagctagcaggtttggccgtgtagtgctacccagcgctggctgcctcctcagcattgcaattcctctcccatctgggcaccagtcagctaccctggtgggaatctgggtagccctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 56 - DNA, 1482 bp, Artificial Sequence
  Description of Artificial Sequence Synthetic polynucleotide
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcaattcacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttgaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtgtgctgggcggggggcggcgggccctcccgcagaacaccatgcgctcttcggaagcaattcagtctcgttgtgcctgtgacctggtacaacgagactgaattgcttctgaggagcgccttgacagcagccatgggagggccgccccctacctcagtgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 57 - DNA, 4606 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcgtcgacataacgcgtatatctagacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctagtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattccccggggatccaccgtgctgggcggggggcggcgggccctcccgcagaacaccatgcgctccacggaacctgctagctccatgcttgctgtggccactgagaagtaagcatggagctagcaggttctgaggagcgccttgacagcagccatgggagggccgccccctacctcagtgactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttactagatttaaataattcgcccttgggcctaggcaattggatccgccggcagagaaaacatcccagggatttacagatcacatgcaggcagggaccagctcaacccttctttaatgtcatccagggagggggccagggatggaggggaggggttgaggagcgagaggcagttatttttgggtgggattcaccacttttcccatgaagaggggagacttggtattttgttcaatcattaagaagacaaagggtttgttgaacttgacctcgggggggatagacatgggtatggcctctaaaaacatggccccagcagcttcagtccctttctcgtcgatggtcagcacagccttatgcacggcctggaggggagagaagcagagacacgttgtaaggctgatcccaggcctcgagcaaggctcacgtggacacctcccaggaagcgctcactccccctggacggccctggccctgcacatcctctccctccctgtcacataggccttgctcctcctcaaggctttggctgatggggctggctcccctctgtccatcttcctgacaagcgcctctccccctgctcaggtgcacccacaactcagaacagggaagagcatcgtcactccacgtctgcctccagggctctctcctttctagtacacggcttgaagctccttgaggacacggaccctggcagtgaccttcacagtgcccagaccccaagataatgcagccattcatggaactgcaggttgttcattggtcgcctttagttttccaaaataagtgtcactttagctgaaatcattcattaattcagacaccaaatctcacagatcgaaggagtcagaaattcctttgaaacaacttagcccaaacctttctgtgtcagtatggataaatcaaggcccaatgtctagaaggtcttgggcaaagttgaaattcagggtcagtgacacaacctcaagggaggccccgaaagtgccagctgcacagcagcccctgcctggctttgctgtttgcccaccgtcccgtgtcagtgaatcacgggcatcttcaggagctcagcctgggtcttcatttgtttccctcggccccttcctcagcctcaggacagtgctgcagcccccacacattcttccctacagataccatggtgcaacaaggtcgtcagggtgatctcaccttggagagcttcaggggtgcctcctctgtgaccccggagaggtcagccccattgctgaagaccttagtgatgcccagttgacccaggacgctcttcagatcataggttccagtaatggacagtttgggtaaatgtaagctggcagacctgtcgtgcagaaaagaaattcaaggcatggcacagcattcctcttgttcttctgggacccaccacagtgcaagtgttttcttttctgattatttctgccacttactcctgtgtcctccacccacactaagatgggaactcggctttggtttgttctacttttagctcttctacattgagtcaaagaatgttaacatcgaatgaatcacaaaagcttgaaatgccacctcctctgatattctaggtgtcctggaagcctgtctcatcttgccctgtagtgttgggtcacctggcccccagcctgtaacatccccagggccctacacccagagaaacacggggctggtggcagtgcccagtgacaaccgtttagtggataagagaagagtgaccacaccaggctgagtgctcctctctggttttccatggggagacaatgccaccctgagcagggtctggtgtgagcggcagctggctctgggctctctgatccgttaccctctcagcctctttgttctttctcaacccctggagcagagacctcaggaggtgctggcatggaacagagaaattccagcctcgattcctattatgaacccgacaccttttgtattttcatcttggttttacagtgtacaaaacgaactagatcagcagggcatgggcataatcacgaatgcacacacatacactaatgtgtggctcatgtttaagtatcacttactacaggacacccaatctaacagcaccgataaagtgacagagaaacgcaagccttctgcgaacatggcctggctgttccaattccgaaccttgcttttctgggccttgccacacaggctcttcccccgtccccccagggacattctacccttgaactccacactccactgctgcctttgccaggaagcccatctgttcctttttggttctgccagaacgtgtggtggtgctgctgtccctgccttgggcactggatattgggaagggacagtgtccacactggagtgggaagttcccagggacgagacctttacctcctcaccctgggtactgttctcctcatggagcatggatttaaatatgtcgtgcatcgatgctacgtagataagtagcatggcgggttaatcattaactacagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 58 - DNA, 2308 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcgtcgacataacgcgtatatctagacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctagtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattccccggggatccaccgtgctgggcggggggcggcgggccctcccgcagaacaccatgcgctccacggaacctgctagctccatgcttgctgtggccactgagaagtaagcatggagctagcaggttctgaggagcgccttgacagcagccatgggagggccgccccctacctcagtgactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttactagatttaaataattcgcccttgggcctaggcaattggatccgccggcagagaaaacatcccagggatttacagatcacatgcaggcagggaccagctcaacccttctatttaaatatgtcgtgcatcgatgctacgtagataagtagcatggcgggttaatcattaactacagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 59 - DNA, 4608 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcgtcgacataacgcgtaattcgcccttgggcctaggcaattggatccgccggcagagaaaacatcccagggatttacagatcacatgcaggcagggaccagctcaacccttctttaatgtcatccagggagggggccagggatggaggggaggggttgaggagcgagaggcagttatttttgggtgggattcaccacttttcccatgaagaggggagacttggtattttgttcaatcattaagaagacaaagggtttgttgaacttgacctcgggggggatagacatgggtatggcctctaaaaacatggccccagcagcttcagtccctttctcgtcgatggtcagcacagccttatgcacggcctggaggggagagaagcagagacacgttgtaaggctgatcccaggcctcgagcaaggctcacgtggacacctcccaggaagcgctcactccccctggacggccctggccctgcacatcctctccctccctgtcacataggccttgctcctcctcaaggctttggctgatggggctggctcccctctgtccatcttcctgacaagcgcctctccccctgctcaggtgcacccacaactcagaacagggaagagcatcgtcactccacgtctgcctccagggctctctcctttctagtacacggcttgaagctccttgaggacacggaccctggcagtgaccttcacagtgcccagaccccaagataatgcagccattcatggaactgcagttgttcattggtcgcctttagttttccaaaataagtgtcatctttagctgaaatcattcattaattcagacaccaaatctcacagatcgaaggagtcagaaattcctttgaaacaacttagcccaaacctttctgtgtcagtatggataaatcaaggcccaatgtctagaaggtcttgggcaaagttgaaattcagggtcagtgacacaacctcaagggaggccccgaaagtgccagctgcacagcagtcccctgcctggctttgctgtttgaccacgtcccgtgtcagtgaatcacgggcatcttcaggagctcagcctgggtcttcatttgtttccctcggccccttcctcagcctcaggacagagctgcagcccccacacattcttccctacagataccagggtgcaacaaggtcgtcagggtgatctcaccttggagagcttcaggggtgcctcctctgtgaccccggagaggtcagccccattgctgaagaccttagtgatgcccagttgacccaggacgctcttcagatcataggttccagtaatggacagtttgggtaaatgtaagctggcagacctgtcgtgcagaaaagaaattcaaggcatggcacagcattcctcttgttcttctgggacccaccacagtgcaagtgttttcttttctgattatttctgccacttactcctgtgtcctccacccacactaagatgggaactcggctttggtttgttctacttttagctcttctacattgagtcaaagaatgttaacatcgaatgaatcacaaaagcttgaaatgccacctcctctgatattctaggtgtcctggaagcctgtctcatcttgccctgtagtgttgggtcacctggcccccagcctgtaacatccccagggccctacacccagagaaacacggggctggtggcagtgcccagtgacaaccgtttagtggataagagaagagtgaccacaccaggctgagtgctcctctctggttttccatgggggagacaatgccaccctgagcagggtctggtgtgagcgggcagcttggctctgggctctctgatccgttaccctctcagcctctttgttctttctcaacccctggagcagagacctcaggaggtgctggcatggaacagagaaattccagcctcgattcctattatgaacccgacaccttttgtattttcatcttggttttacagtgtacaaaacgaactagatcagcagggcatgggcataatcacgaatgcacacacatacactaatgtgtggctcatgtttaagtatcacttactacaggacacccaatctaacagcaccgataaagtgacagagaaacgcaagccttctgcgaacatggcctggctgttccaattccgaaccttgcttttctgggccttgccacacaggctcttcccccgtccccccagggacattctacccttgaactccacactccactgctgcctttgccaggaagcccatctgttcctttttggttctgccagaacgtgtggtggtgctgctgtccctgccctgggcactggatattgggaagggacagtgtccacactggagtgggaagtccccagggacgagacctttacctcctcacccctgggtactgttctcctcatggagcatggacgcgtatatctagacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctagtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattccccggggatccaccgtgctgggcggggggcggcgggccctcccgcagaacaccatgcgctccacggaacctgctagctccatgcttgctgtggccactgagaagtaagcatggagctagcaggttctgaggagcgccttgacagcagccatgggagggccgccccctacctcagtgactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttactagatttaaatatgtcgtgcatcgatgctacgtagataagtagcatggcgggttaatcattaactacagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 60 - DNA, 2306 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcgtcgacataacgcgtaattcgcccttgggcctaggcaattggatccgccggcagagaaaacatcccagggatttacagatcacatgcaggcagggaccagctcaacccttctacgcgtatatctagacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctagtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattccccggggatccaccgtgctgggcggggggcggcgggccctcccgcagaacaccatgcgctccacggaacctgctagctccatgcttgctgtggccactgagaagtaagcatggagctagcaggttctgaggagcgccttgacagcagccatgggagggccgccccctacctcagtgactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttactagatttaaatatgtcgtgcatcgatgctacgtagataagtagcatggcgggttaatcattaactacagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 61 - DNA, 4598 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcgtcgacataacgcgtatatctagacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctagtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaattcgcccttgggcctaggcaattggatccgccggcagagaaaacatcccagggatttacagatcacatgcaggcagggaccagctcaacccttctttaatgtcatccagggagggggccagggatggaggggaggggttgaggagcgagaggcagttatttttgggtgggattcaccacttttcccatgaagaggggagacttggtattttgttcaatcattaagaagacaaagggtttgttgaacttgacctcgggggggatagacatgggtatggcctctaaaaacatggccccagcagcttcagtccctttctcgtcgatggtcagcacagccttatgcacggcctggaggggagagaagcagagacacgttgtaaggctgatcccaggcctcgagcaaggctcacgtggacacctcccaggaagcgctcactccccctggacggccctggccctgcacatcctctccctccctgtcacataggccttgctcctcctcaaggctttggctgatggggctggctcccctctgtccatcttcctgacaagcgcctctccccctgctcaggtgcacccacaactcagaacagggaagagcatcgtcactccacgtctgcctccagggctctctcctttctagtacacggcttgaagctccttgaggacacggaccctggcagtgaccttcacagtgcccagaccccaagataatgcagccattcatggaactgcaggttgttcattggtcgcctttagttttccaaaataagtgtcactttagctgaaatcattcattaattcagacaccaaatctcacagatcgaaggagtcagaaattcctttgaaacaacttagcccaaacctttctgtgtcagtatggataaatcaaggcccaatgtctagaaggtcttgggcaaagttgaaattcagggtcagtgacacaacctcaagggaggccccgaaagtgccagctgcacagcagcccctgcctggctttgctgtttgcccaccgtcccgtgtcagtgaatcacgggcatcttcaggagctcagcctgggtcttcatttgtttccctcggccccttcctcagcctcaggacagtgctgcagcccccacacattcttccctacagataccatggtgcaacaaggtcgtcagggtgatctcaccttggagagcttcaggggtgcctcctctgtgaccccggagaggtcagccccattgctgaagaccttagtgatgcccagttgacccaggacgctcttcagatcataggttccagtaatggacagtttgggtaaatgtaagctggcagacctgtcgtgcagaaaagaaattcaaggcatggcacagcattcctcttgttcttctgggacccaccacagtgcaagtgttttcttttctgattatttctgccacttactcctgtgtcctccacccacactaagatgggaactcggctttggtttgttctacttttagctcttctacattgagtcaaagaatgttaacatcgaatgaatcacaaaagcttgaaatgccacctcctctgatattctaggtgtcctggaagcctgtctcatcttgccctgtagtgttgggtcacctggcccccagcctgtaacatccccagggccctacacccagagaaacacggggctggtggcagtgcccagtgacaaccgtttagtggataagagaagagtgaccacaccaggctgagtgctcctctctggttttccatggggagacaatgccaccctgagcagggtctggtgtgagcggcagctggctctgggctctctgatccgttaccctctcagcctctttgttctttctcaacccctggagcagagacctcaggaggtgctggcatggaacagagaaattccagcctcgattcctattatgaacccgacaccttttgtattttcatcttggttttacagtgtacaaaacgaactagatcagcagggcatgggcataatcacgaatgcacacacatacactaatgtgtggctcatgtttaagtatcacttactacaggacacccaatctaacagcaccgataaagtgacagagaaacgcaagccttctgcgaacatggcctggctgttccaattccgaaccttgcttttctgggccttgccacacaggctcttcccccgtccccccagggacattctacccttgaactccacactccactgctgcctttgccaggaagcccatctgttcctttttggttctgccagaacgtgtggtggtgctgctgtccctgccttgggcactggatattgggaagggacagtgtccacactggagtgggaagttcccagggacgagacctttacctcctcaccctgggtactgttctcctcatggagcatggaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattccccggggatccaccgtgctgggcggggggcggcgggccctcccgcagaacaccatgcgctccacggaacctgctagctccatgcttgctgtggccactgagaagtaagcatggagctagcaggttctgaggagcgccttgacagcagccatgggagggccgccccctacctcagtgactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttactagatttaaatatgtcgtgcatcgatgctacgtagataagtagcatggcgggttaatcattaactacagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 62 - DNA, 2300 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcgtcgacataacgcgtatatctagacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctagtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaattcgcccttgggcctaggcaattggatccgccggcagagaaaacatcccagggatttacagatcacatgcaggcagggaccagctcaacccttctaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattccccggggatccaccgtgctgggcggggggcggcgggccctcccgcagaacaccatgcgctccacggaacctgctagctccatgcttgctgtggccactgagaagtaagcatggagctagcaggttctgaggagcgccttgacagcagccatgggagggccgccccctacctcagtgactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttactagatttaaatatgtcgtgcatcgatgctacgtagataagtagcatggcgggttaatcattaactacagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 63 - DNA, 4615 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcgtcgacataacgcgtaattcgcccttgggcctaggcaattggatccgccggcagagaaaacatcccagggatttacagatcacatgcaggcagggaccagctcaacccttctttaatgtcatccagggagggggccagggatggaggggaggggttgaggagcgagaggcagttatttttgggtgggattcaccacttttcccatgaagaggggagacttggtattttgttcaatcattaagaagacaaagggtttgttgaacttgacctcgggggggatagacatgggtatggcctctaaaaacatggccccagcagcttcagtccctttctcgtcgatggtcagcacagccttatgcacggcctggaggggagagaagcagagacacgttgtaaggctgatcccaggcctcgagcaaggctcacgtggacacctcccaggaagcgctcactccccctggacggccctggccctgcacatcctctccctccctgtcacataggccttgctcctcctcaaggctttggctgatggggctggctcccctctgtccatcttcctgacaagcgcctctccccctgctcaggtgcacccacaactcagaacagggaagagcatcgtcactccacgtctgcctccagggctctctcctttctagtacacggcttgaagctccttgaggacacggaccctggcagtgaccttcacagtgcccagaccccaagataatgcagccattcatggaactgcagttgttcattggtcgcctttagttttccaaaataagtgtcatctttagctgaaatcattcattaattcagacaccaaatctcacagatcgaaggagtcagaaattcctttgaaacaacttagcccaaacctttctgtgtcagtatggataaatcaaggcccaatgtctagaaggtcttgggcaaagttgaaattcagggtcagtgacacaacctcaagggaggccccgaaagtgccagctgcacagcagtcccctgcctggctttgctgtttgaccacgtcccgtgtcagtgaatcacgggcatcttcaggagctcagcctgggtcttcatttgtttccctcggccccttcctcagcctcaggacagagctgcagcccccacacattcttccctacagataccagggtgcaacaaggtcgtcagggtgatctcaccttggagagcttcaggggtgcctccacgcgtatatctagacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctagtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattccccggggatccaccgtgctgggcggggggcggcgggccctcccgcagaacaccatgcgctccacggaacctgctagctccatgcttgctgtggccactgagaagtaagcatggagctagcaggttctgaggagcgccttgacagcagccatgggagggccgccccctacctcagtgactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttactagatttaaattctgtgaccccggagaggtcagccccattgctgaagaccttagtgatgcccagttgacccaggacgctcttcagatcataggttccagtaatggacagtttgggtaaatgtaagctggcagacctgtcgtgcagaaaagaaattcaaggcatggcacagcattcctcttgttcttctgggacccaccacagtgcaagtgttttcttttctgattatttctgccacttactcctgtgtcctccacccacactaagatgggaactcggctttggtttgttctacttttagctcttctacattgagtcaaagaatgttaacatcgaatgaatcacaaaagcttgaaatgccacctcctctgatattctaggtgtcctggaagcctgtctcatcttgccctgtagtgttgggtcacctggcccccagcctgtaacatccccagggccctacacccagagaaacacggggctggtggcagtgcccagtgacaaccgtttagtggataagagaagagtgaccacaccaggctgagtgctcctctctggttttccatggggagacaatgccaccctgagcagggtctggtgtgagcgggcagcttggctctgggctctctgatccgttaccctctcagcctctttgttctttctcaacccctggagcagagacctcaggaggtgctggcatggaacagagaaattccagcctcgattcctattatgaacccgacaccttttgtattttcatcttggttttacagtgtacaaaacgaactagatcagcagggcatgggcataatcacgaatgcacacacatacactaatgtgtggctcatgtttaagtatcacttactacaggacacccaatctaacagcaccgataaagtgacagagaaacgcaagccttctgcgaacatggcctggctgttccaattccgaaccttgcttttctgggccttgccacacaggctcttcccccgtccccccagggacattctacccttgaactccacactccactgctgcctttgccaggaagcccatctgttcctttttggttctgccagaacgtgtggtggtgctgctgtccctgccctgggcactggatattgggaagggacagtgtccacactggagtgggaagtccccagggacgagacctttacctcctcacccctgggtactgttctcctcatggagcatggatttaaatatgtcgtgcatcgatgctacgtagataagtagcatggcgggttaatcattaactacagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 64 - DNA, 2314 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcgtcgacataacgcgtaattcgcccttgggcctaggcaattggatccgccggcagagaaaacatcacgcgtatatctagacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctagtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattccccggggatccaccgtgctgggcggggggcggcgggccctcccgcagaacaccatgcgctccacggaacctgctagctccatgcttgctgtggccactgagaagtaagcatggagctagcaggttctgaggagcgccttgacagcagccatgggagggccgccccctacctcagtgactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctgactagatttaaatccagggatttacagatcacatgcaggcagggaccagctcaactcttctatttaaatatgtcgtgcatcgatgctacgtagataagtagcatggcgggttaatcattaactacagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 65 - DNA, 2198 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtatacatgcgtcgacataacgcgtatatctagacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctagtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattccccggggatccaccgtgctgggcggggggcggcgggccctcccgcagaacaccatgcgctcttcggaagcaattcagtctcgttgtgcctgtgacctggtacaacgagactgaattgcttctgaggagcgccttgacagcagccatgggagggccgccccctacctcagtgactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttactagatttaaatatgtcgtgcatcgatgctacgtagataagtagcatggcgggttaatcattaactacagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 66 - DNA, 2203 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtatacatgcgtcgacataacgcgtatatctagacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctagtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattccccggggatccaccgtgctgggcggggggcggcgggccctcccgcagaacaccatgcgctccacggaacctgctagctccatgcttgctgtggccactgagaagtaagcatggagctagcaggttctgaggagcgccttgacagcagccatgggagggccgccccctacctcagtgactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttactagatttaaatatgtcgtgcatcgatgctacgtagataagtagcatggcgggttaatcattaactacagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 67 - DNA, 844 bp, Artificial Sequence
  Description of Artificial Sequence Synthetic polynucleotide
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcaattcacgcgttgacgtacgaaattcgaacgctgacgtcatcaacccgctccaaggaatcgcgggcccagtgtcactaggcgggaacacccagcgcgcgtgcgccctggcaggaagatggctgtgagggacagggagtggcgccctgcaatatttgcatgtcgctatgtgttctgggaaatcaccataaacgtgaaatgtctttggatttgggaatcttataagttctgtatgagaccacaccggtgaagcaaagaaggggcagagggagcccgtgagctgagtgggccagggactgggagaaggagtgaggaggcagggccggcatgcctctgctgctggccagacctgctagctccatgcttcccgtctgcacctgtcactagtaagcatggagctagcaggtttggccgtgtagtgctacccagcgctggctgcctcctcagcattgcaattcctctcccatctgggcaccagtcagctaccctggtgggaatctgggtagccctcgagatagcggccgcccccgagatctttttatgtacatctagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 68 - DNA, 742 bp, Artificial Sequence
  Description of Artificial Sequence Synthetic polynucleotide
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcaattcacgcgttgacgtacgaaattcgaacgctgacgtcatcaacccgctccaaggaatcgcgggcccagtgtcactaggcgggaacacccagcgcgcgtgcgccctggcaggaagatggctgtgagggacagggagtggcgccctgcaatatttgcatgtcgctatgtgttctgggaaatcaccataaacgtgaaatgtctttggatttgggaatcttataagttctgtatgagaccacaccggtgtgctgggcggggggcggcgggccctcccgcagaacaccatgcgctcttcggaagcaattcagtctcgttgtgcctgtgacctggtacaacgagactgaattgcttctgaggagcgccttgacagcagccatgggagggccgccccctacctcagtgactcgagatagcggccgcccccgagatctttttatgtacatctagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 69 - DNA, 1482 bp, Artificial Sequence
  Description of Artificial Sequence Synthetic polynucleotide
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcaattcacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttgaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtgtgctgggcggggggcggcgggccctcccgcagaacaccatgcgctcttcggaacctgctagctccatgcttgcctgtgacctggtaagcatggagctagcaggttctgaggagcgccttgacagcagccatgggagggccgccccctacctcagtgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 70 - DNA, 1482 bp, Artificial Sequence
  Description of Artificial Sequence Synthetic polynucleotide
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcaattcacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttgaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtgtgctgggcggggggcggcgggccctcccgcagaacaccatgcgctcttcggaagcaattcagtctcgttgtccctgtgacctggtacaacgagactgaattgcttctgaggagcgccttgacagcagccatgggagggccgccccctacctcagtgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 71 - DNA, 1482 bp, Artificial Sequence
  Description of Artificial Sequence Synthetic polynucleotide
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcaattcacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttgaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtgtgctgggcggggggcggcgggccctcccgcagaacaccatgcgctcttcggaatgaacctcttgttataaagcctgtgacctggttttataacaagaggttcattctgaggagcgccttgacagcagccatgggagggccgccccctacctcagtgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 72 - DNA, 1482 bp, Artificial Sequence
  Description of Artificial Sequence Synthetic polynucleotide
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcaattcacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttgaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtgtgctgggcggggggcggcgggccctcccgcagaacaccatgcgctcttcggaaccgacaaccagtatttgggcctgtgacctggtccaaatactggttgtcggttctgaggagcgccttgacagcagccatgggagggccgccccctacctcagtgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 73 - DNA, 1482 bp, Artificial Sequence
  Description of Artificial Sequence Synthetic polynucleotide
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcaattcacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttgaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtgtgctgggcggggggcggcgggccctcccgcagaacaccatgcgctcttcggaaccacaatgttgtgactgggcctgtgacctggtccggtcacaacattgtggttctgaggagcgccttgacagcagccatgggagggccgccccctacctcagtgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 74 - DNA, 1482 bp, Artificial Sequence
  Description of Artificial Sequence Synthetic polynucleotide
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcaattcacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttgaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtgtgctgggcggggggcggcgggccctcccgcagaacaccatgcgctcttcggaagcaattcagtctcgttgtacctgtgatttggtacaacgagactgaattgcttctgaggagcgccttgacagcagccatgggagggccgccccctacctcagtgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 75 - DNA, 1482 bp, Artificial Sequence
  Description of Artificial Sequence Synthetic polynucleotide
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcaattcacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttgaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtgtgctgggcggggggcggcgggccctcccgcagaacaccatgcgctcttcggaagcaattcagactcgttgtacctgtgacctggtacaacgagactgaattgcttctgtggagcgccttgacagcagccatgggagggccgccccctacctcagtgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 76 - DNA, 1482 bp, Artificial Sequence
  Description of Artificial Sequence Synthetic polynucleotide
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcaattcacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttgaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtgtgctgggcggggggcggcgggccctcccgcagaacaccatgcgctcttcggaagcaattcagtctcgttgtacctgtgacctggtacaacgagactgaattgcttctgtggagcgccttgacagcagccatgggagggccgccccctacctcagtgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 77 - DNA, 1584 bp, Artificial Sequence
  Description of Artificial Sequence Synthetic polynucleotide
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcaattcacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttgaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtgaagcaaagaaggggcagagggagcccgtgagctgagtgggccagggactgggagaaggagtgaggaggcagggccggcatgcctctgctgctggccagagcaattcagtctcgttgtaccgtctgcacctgtcactagtacaacgagactgaattgctttggccgtgtagtgctacccagcgctggctgcctcctcagcattgcaattcctctcccatctgggcaccagtcagctaccctggtgggaatctgggtagccctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 78 - DNA, 1584 bp, Artificial Sequence
  Description of Artificial Sequence Synthetic polynucleotide
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcaattcacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttgaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtgaagcaaagaaggggcagagggagcccgtgagctgagtgggccagggactgggagaaggagtgaggaggcagggccggcatgcctctgctgctggccagaggtttatgaactgacgttaccgtctgcacctgtcactagtaacgtcagttcataaacctttggccgtgtagtgctacccagcgctggctgcctcctcagcattgcaattcctctcccatctgggcaccagtcagctaccctggtgggaatctgggtagccctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 79 - DNA, 1584 bp, Artificial Sequence
  Description of Artificial Sequence Synthetic polynucleotide
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcaattcacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttgaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtgaagcaaagaaggggcagagggagcccgtgagctgagtgggccagggactgggagaaggagtgaggaggcagggccggcatgcctctgctgctggccagaccgacaaccagtatttggaccgtctgcacctgtcactagtccaaatactggttgtcggtttggccgtgtagtgctacccagcgctggctgcctcctcagcattgcaattcctctcccatctgggcaccagtcagctaccctggtgggaatctgggtagccctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 80 - DNA, 1584 bp, Artificial Sequence
  Description of Artificial Sequence Synthetic polynucleotide
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcaattcacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttgaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtgaagcaaagaaggggcagagggagcccgtgagctgagtgggccagggactgggagaaggagtgaggaggcagggccggcatgcctctgctgctggccagaccacaatgttgtgactggaccgtctgcacctgtcactagtccggtcacaacattgtggtttggccgtgtagtgctacccagcgctggctgcctcctcagcattgcaattcctctcccatctgggcaccagtcagctaccctggtgggaatctgggtagccctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 81 - DNA, 1487 bp, Artificial Sequence
  Description of Artificial Sequence Synthetic polynucleotide
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcaattcacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttgaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtgtgctgggcggggggcggcgggccctcccgcagaacaccatgcgctccacggaacctgctagctccatgcttgctgtggccactgagaagtaagcatggagctagcaggttctgaggagcgccttgacagcagccatgggagggccgccccctacctcagtgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 82 - DNA, 1487 bp, Artificial Sequence
  Description of Artificial Sequence Synthetic polynucleotide
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcaattcacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttgaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtgtgctgggcggggggcggcgggccctcccgcagaacaccatgcgctcctcggaataagcatggagctagcaggttgtggccactgagaaacctgctagctccatgcttgttctgcggagcgccttgacagcagccatgggagggccgccccctacctcagtgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

(19) United States
(12) Patent Application Publication
(10) Pub. No.: US 2021/0355454 A1
(43) Pub. Date: Nov. 18, 2021

(54) SYSTEMS AND METHODS FOR PRODUCING GENE THERAPY FORMULATIONS

(71) Applicant: VOYAGER THERAPEUTICS, INC., Cambridge, MA (US)

(72) Inventors: Jacob J. Cardinal, North Cambridge, MA (US); Robert Steininger, Cambridge, MA (US); Lori B. Karpes, Ashland, MA (US); Christopher J. Morrison, Arlington, MA (US); Daniel S. Hurwit, Seattle, WA (US); Matthew Luther, Cambridge, MA (US); Andrew M. Wood, Concord, MA (US); Dinah Wen-Yee Sah, Cambridge, MA (US); Pengcheng Zhou, Lexington, MA (US); Jeffrey S. Thompson, Stoneham, MA (US); Christina Gamba-Vitalo, Cambridge, MA (US); Jenna Carroll Soper, Winchester, MA (US); Steven M. Hersch, Jamaica Plain, MA (US); Todd Carter, Cambridge, MA (US)

(21) Appl. No.: 17/262,271

(22) PCT Filed: Jul. 24, 2019
(86) PCT No.: PCT/US2019/043196

§ 371 Date: Jan. 22, 2021

Related U.S. Application Data
(60) Provisional application No. 62/702,687, filed on Jul. 24, 2018.
(60) Provisional application No. 62/702,679, filed on Jul. 24, 2018.
(60) Provisional application No. 62/725,432, filed on Aug. 31, 2018.
(60) Provisional application No. 62/741,508, filed on Oct. 4, 2018.
(60) Provisional application No. 62/794,213, filed on Jan. 18, 2019.
(60) Provisional application No. 62/794,212, filed on Jan. 18, 2019.
(60) Provisional application No. 62/794,199, filed on Jan. 18, 2019.
(60) Provisional application No. 62/826,363, filed on Mar. 29, 2019.
(60) Provisional application No. 62/839,880, filed on Apr. 29, 2019.

(51) Int. Cl. C12N 7/02; C12N 15/86; A61K 35/76; A61P 25/14

(52) U.S. Cl. CPC C12N 7/02; A61P 25/14; A61K 35/76; C12N 15/86

(57) ABSTRACT

The present disclosure describes methods and systems for use in the production of adeno-associated virus (AAV) particles and AAV formulations, including recombinant adeno-associated virus (rAAV) particles and formulations. In certain embodiments, the present disclosure presents methods and systems for clarifying, purifying, formulating, filtering and processing AAV particles and AAV formulations. The present disclosure also describes compositions, methods and processes for the design, preparation, manufacture, use and/or formulation of AAV particles comprising modulatory polynucleotides, e.g., polynucleotides encoding small interfering RNA (siRNA) molecules which target the Huntingtin (HTT) gene (e.g., the wild-type or the mutated CAG-expanded HTT gene). Methods for using formulated AAV particles comprising modulatory polynucleotides to inhibit the HTT gene expression in a subject with a neurodegenerative disease (e.g., Huntington's Disease (HD)) are also disclosed.

### CROSS REFERENCE TO RELATED APPLICATIONS

This application claims the benefit of: U.S. Provisional Patent Application No. 62/702,687, filed Jul. 24, 2018, entitled GENE THERAPY FORMULATIONS; U.S. Provisional Patent Application No. 62/702,679, filed Jul. 24, 2018, entitled COMPOSITIONS AND METHODS FOR TREATING HUNTINGTON'S DISEASE; U.S. Provisional Patent Application No. 62/725,432, filed Aug. 31, 2018, entitled COMPOSITIONS AND METHODS FOR TREATING HUNTINGTON'S DISEASE; U.S. Provisional Patent Application No. 62/741,508, filed Oct. 4, 2018, entitled SYSTEMS AND METHODS FOR CLARIFYING GENE THERAPY FORMULATIONS; U.S. Provisional Patent Application No. 62/794,199, filed Jan. 18, 2019, entitled METHODS AND SYSTEMS FOR PRODUCING AAV PARTICLES; U.S. Provisional Patent Application No. 62/794,212, filed Jan. 18, 2019, entitled SYSTEMS AND METHODS FOR CLARIFYING GENE THERAPY FORMULATIONS; U.S. Provisional Patent Application No. 62/794,213, filed Jan. 18, 2019, entitled FORMULATIONS FOR AAV PARTICLES; U.S. Provisional Patent Application No. 62/826,363, filed Mar. 29, 2019, entitled SYSTEMS AND METHODS FOR CLARIFYING AND PURIFYING GENE THERAPY FORMULATIONS; U.S. Provisional Patent Application No. 62/839,880, filed Apr. 29, 2019, entitled COMPOSITIONS AND METHODS FOR TREATING HUNTINGTON'S DISEASE; the contents of which are each incorporated herein by reference in their entirety.

### REFERENCE TO THE SEQUENCE LISTING

The present application is being filed along with a Sequence Listing in electronic format. The Sequence Listing is provided as a file entitled 20571527PCTSL.txt, created on Jul. 24, 2019, which is 352,382 bytes in size. The information in the electronic format of the sequence listing is incorporated herein by reference in its entirety.

### FIELD OF THE DISCLOSURE

The present disclosure describes methods and systems for use in the production of adeno-associated virus (AAV) particles and AAV formulations, including recombinant adeno-associated virus (rAAV) particles and formulations. In certain embodiments, the present disclosure presents methods and systems for clarifying, purifying, formulating, filtering and processing AAV particles and AAV formulations.

The present disclosure also describes compositions, methods and processes for the design, preparation, manufacture, use and/or formulation of AAV particles comprising modulatory polynucleotides, e.g., polynucleotides encoding small interfering RNA (siRNA) molecules which target the Huntingtin (HT) gene (e.g., the wild-type or the mutated CAG-expanded HTT gene). Methods for using formulated AAV particles comprising modulatory polynucleotides to inhibit the HTT gene expression in a subject with a neurodegenerative disease (e.g., Huntington's Disease (HD)) are also disclosed.

### BACKGROUND

AAVs have emerged as one of the most widely studied and utilized viral vectors for gene transfer to mammalian cells. See, e.g., Tratschin et al., Mol. Cell Biol., 5(11):3251-3260 (1985) and Grimm et al., Hum. Gene Ther., 10(15):2445-2450 (1999), the contents of which are herein incorporated by reference in their entirety. Adeno-associated viral (AAV) vectors are promising candidates for therapeutic gene delivery and have proven safe and efficacious in clinical trials. The design and production of improved AAV particles for this purpose is an active field of study.

With the advent of development in the AAV field, there remains a need for improved systems and methods for producing AAV vectors (such as AAV particles) and corresponding therapeutic formulations for storage and delivery of the AAV particles. These include improved methods and systems for clarifying, purifying, formulating, filtering and processing AAV particles and AAV formulations

### SUMMARY

The present disclosure presents methods and systems for producing a pharmaceutical formulation. In certain embodiments, the pharmaceutical formulation comprises adeno-associated virus (AAV) particles. In certain embodiments, the methods include one or more steps selected from: chemical lysis, clarification filtration, affinity chromatography, ion-exchange chromatography, tangential flow filtration (TFF), and virus retentive filtration.

In certain embodiments, the present disclosure presents a method or process for producing a pharmaceutical formulation comprising adeno-associated virus (AAV) particles. In certain embodiments, the method includes: Producing AAV particles in one or more viral production cells (VPCs) within a bioreactor, thereby providing a viral production pool which includes the AAV particles and a liquid media; Processing the viral production pool through one or more steps selected from: chemical lysis, clarification filtration, affinity chromatography, ion-exchange chromatography, tangential flow filtration (TFF), and virus retentive filtration; and Incorporating the AAV particles from the viral production pool into a pharmaceutical formulation, wherein the pharmaceutical formulation includes the AAV particles and at least one pharmaceutical excipient. In certain embodiments, the method includes one or more chemical lysis steps in which the viral production pool is exposed to chemical lysis. In certain embodiments, the method includes one or more clarification filtration steps in which the viral production pool is processed through one or more clarification filtration systems. In certain embodiments, the method includes one or more affinity chromatography steps in which the viral production pool is processed through one or more affinity chromatography systems. In certain embodiments, the method includes one or more ion exchange chromatography steps in which the viral production pool is processed through one or more ion exchange chromatography systems. In certain embodiments, the method includes one or more tangential flow filtration (TFF) steps in which the viral production pool is processed through one or more TFF systems. In certain embodiments, the method includes one or more virus retentive filtration (VRF) steps in which the viral production pool is processed through one or more VRF systems.

In certain embodiments, the AAV particles are produced in viral production cells (VPCs) within a bioreactor. In certain embodiments, the VPCs include insect cells. In certain embodiments, the VPCs include Sf9 insect cells. In certain embodiments, the AAV particles are produced using a baculovirus production system.

In certain embodiments, the method includes one or more chemical lysis steps in which the viral production pool is exposed to chemical lysis. In certain embodiments, the method includes: Collecting the viral production pool from the bioreactor, wherein the viral production pool includes the one or more VPCs, and wherein the AAV particles are contained within the VPCs; and Exposing the VPCs within the viral production pool to chemical lysis using a chemical lysis solution under chemical lysis conditions, wherein the chemical lysis releases the AAV particles from the VPCs into the liquid media of the viral production pool. In certain embodiments, the chemical lysis solution comprises a stabilizing additive selected from arginine or arginine salts. In certain embodiments, the concentration of the stabilizing additive is between 0.1-0.5 M. In certain embodiments, the concentration of the stabilizing additive is between 0.2-0.3 M.

In certain embodiments, the chemical lysis solution does not include Triton X-100. In certain embodiments, the chemical lysis solution includes a zwitterionic detergent selected from Lauryl dimethylamine N-oxide (LDAO); N,N-Dimethyl-N-dodecylglycine betaine (Empigen BB); 3-(N,N-Dimethyl myristylammonio) propanesulfonate (Zwittergent 3-10); n-Dodecyl-N,N-dimethyl-3-ammonio-1-propanesulfonate (Zwittergent 3-12); n-Tetradecyl-N,N-dimethyl-3-ammonio-1-propanesulfonate (Zwittergent 3-14); 3-(N,N-Dimethyl palmitylammonio) propanesulfonate (Zwittergent 3-16); 3-((3-cholamidopropyl) dimethylammonio)-1-propanesulfonate (CHAPS); or 3-([3-Cholamidopropyl]dimethylammonio)-2-hydroxy-1-propanesulfonate (CHAPSO). In certain embodiments, the chemical lysis solution includes Lauryl dimethylamine N-oxide (LDAO). In certain embodiments, the chemical lysis solution includes N,N-Dimethyl-N-dodecylglycine betaine (Empigen BB).

In certain embodiments, the method includes one or more clarification filtration steps in which the viral production pool is processed through one or more clarification filtration systems. In certain embodiments, the one or more clarification filtration systems include a depth filtration system. In certain embodiments, the depth filtration system includes a Millipore Millistak D0HC media series filter. In certain embodiments, the depth filtration system includes a Millipore Millistak C0SP media series filter. In certain embodiments, the one or more clarification filtration systems include a 0.2 μm microfiltration system.

In certain embodiments, the method includes one or more affinity chromatography steps in which the viral production pool is processed through one or more affinity chromatography systems. In certain embodiments, the method includes processing the viral production pool through one or more immunoaffinity chromatography systems in bind-elute mode. In certain embodiments, the immunoaffinity chromatography system includes one or more recombinant single-chain antibodies which are capable of binding to one or more AAV capsid variants. In certain embodiments, the immunoaffinity chromatography system is regenerated using a regeneration solution. In certain embodiments, the regeneration solution comprises between 1-3 M of guanidine or a guanidine salt. In certain embodiments, the immunoaffinity chromatography system is regenerated using a regeneration solution which includes 2 M guanidine HCL.

In certain embodiments, the method includes one or more ion exchange chromatography steps in which the viral production pool is processed through one or more ion exchange chromatography systems. In certain embodiments, the method comprises processing the viral production pool through one or more anion exchange chromatography systems in flow-through mode. In certain embodiments, the anion exchange chromatography system includes a stationary phase which binds non-viral impurities, non-AAV viral particles, or a combination thereof. In certain embodiments, the anion exchange chromatography system includes a stationary phase which does not bind to AAV particles. In certain embodiments, the stationary phase of the anion exchange chromatography system includes a quaternary amine functional group. In certain embodiments, the anion exchange chromatography system includes a trimethylammonium ethyl (TMAE) functional group.

In certain embodiments, the method includes one or more tangential flow filtration (TFF) steps in which the viral production pool is processed through one or more TFF systems. In certain embodiments, the TFF system includes a flat-sheet filter comprising a regenerated cellulose cassette. In certain embodiments, the TFF system includes a hollow-fiber filter. In certain embodiments, the TFF system is operated at a transmembrane pressure (TMP) of between 5.5-6.5 PSI, and a target crossflow between 5.5-6.5 L/min/m². In certain embodiments, the TFF system is operated at a transmembrane pressure (TMP) of 6 PSI, and a target crossflow of 6 L/min/m². In certain embodiments, a 50% sucrose mixture is added to the viral production pool prior to the one or more TFF steps. In certain embodiments, the 50% sucrose mixture is added to the viral production pool at a centration between 9-13% v/v. In certain embodiments, the 50% sucrose mixture is added to the viral production pool at a centration between 10-12% v/v. In certain embodiments, the 50% sucrose mixture is added to the viral production pool at a centration of 11% v/v.

In certain embodiments, the one or more TFF steps includes a first diafiltration step in which at least a portion of the liquid media of the viral production pool is replaced with a low-sucrose diafiltration buffer. In certain embodiments, the low-sucrose diafiltration buffer includes between 4-6% w/v of a sugar or sugar substitute and between 150-250 mM of an alkali chloride salt. In certain embodiments, the low-sucrose diafiltration buffer includes between 4.5-5.5% w/v of sucrose and between 210-230 mM sodium chloride. In certain embodiments, the low-sucrose diafiltration buffer comprises 5% w/v of sucrose and 220 mM sodium chloride.

In certain embodiments, the one or more TFF steps comprises an ultrafiltration concentration step, wherein the AAV particles in the viral production pool are concentrated to a target particle concentration. In certain embodiments, the AAV particles in the viral production pool are concentrated to between 1.0×10¹²-5.0×10¹³ vg/mL. In certain embodiments, the AAV particles in the viral production pool are concentrated to between 2.0×10¹²-5.0×10¹² vg/mL. In certain embodiments, the AAV particles in the viral production pool are concentrated to between 1.0×10¹³-5.0×10¹³ vg/mL. In certain embodiments, the AAV particles in the viral production pool are concentrated to between 2.0×10¹³-3.0×10¹³ vg/mL. In certain embodiments, the AAV particles in the viral production pool are concentrated to 2.7×10¹³ vg/mL.

In certain embodiments, the one or more TFF steps includes a final diafiltration step in which at least a portion of the liquid media of the viral production pool is replaced with a high-sucrose formulation buffer. In certain embodiments, the high-sucrose formulation buffer includes between 6-8% w/v of a sugar or sugar substitute and between 90-100 mM of an alkali chloride salt. In certain embodiments, the high-sucrose formulation buffer includes 7% w/v of sucrose and between 90-100 mM sodium chloride. In certain embodiments, the high-sucrose formulation buffer comprises 7% w/v of sucrose, 10 mM Sodium Phosphate, between 95-100 mM sodium chloride, and 0.001% (w/v) Poloxamer 188.

In certain embodiments, the method includes one or more virus retentive filtration (VRF) steps in which the viral production pool is processed through one or more VRF systems. In certain embodiments, the VRF system includes a filter medium which retains particles which are 50 nm or larger. In certain embodiments, the VRF system includes a filter medium which retains particles which are 35 nm or larger. In certain embodiments, the VRF system includes a filter medium which retains particles which are 20 nm or larger.

The present disclosure presents methods and systems for producing a gene therapy product, wherein the method includes: providing a pharmaceutical formulation comprising AAV particles, wherein the pharmaceutical formulation is produced by the method of the present disclosure; and suitably aliquoting the pharmaceutical formulation into a formulation container.

The present disclosure presents pharmaceutical formulations useful for gene therapy modalities. In certain embodiments, the pharmaceutical formulations include AAV particles. In certain embodiments, the pharmaceutical formulations include AAV particles at a concentration less than 5×10¹³ vg/ml. In certain embodiments, the pharmaceutical formulations include AAV particles at a concentration between 1.0×10¹²-5.0×10¹³ vg/mL. In certain embodiments, the pharmaceutical formulations include AAV particles at a concentration between 1.0×10¹²-5.0×10¹² vg/mL. In certain embodiments, the pharmaceutical formulations include AAV particles at a concentration between 1.0×10¹³-5.0×10¹³ vg/mL. In certain embodiments, the pharmaceutical formulations include AAV particles at a concentration of 2.7×10¹³ vg/mL.

In certain embodiments, the pharmaceutical formulations include: (i) AAV particles at a concentration less than 5×10¹³ vg/ml; (ii) one or more salts; (iii) one or more sugars or sugar substitutes; and (iv) one or more buffering agents. In certain embodiments, the pharmaceutical formulation is an aqueous formulation.

In certain embodiments, the pharmaceutical formulations include: (i) AAV particles at a concentration less than 5×10¹³ vg/ml; (ii) sodium chloride; (iii) a sugar or sugar substitute; and (iv) a copolymer. In certain embodiments, the pharmaceutical formulation has a pH between 6.5-8. In certain embodiments, the pharmaceutical formulation has an osmolality of 350-500 mOsm/kg.

In certain embodiments, the pharmaceutical formulation includes at least one AAV particle, sodium chloride, sodium phosphate, potassium phosphate, a sugar or sugar substitute and a copolymer. In certain embodiments, the concentration of sodium chloride is 95 mM. In certain embodiments, the concentration of sodium phosphate is 10 mM. In certain embodiments, the 10 mM sodium phosphate includes 5 mM monobasic sodium phosphate and 5 mM dibasic sodium phosphate. In certain embodiments, the concentration of potassium phosphate is 1.5 mM. In certain embodiments, the concentration of the sugar or sugar substitute is 7% w/v. In certain embodiments, the concentration of the copolymer is 0.001% w/v. In certain embodiments, the sugar is sucrose. In certain embodiments, the copolymer is Poloxamer 188 (e.g., Pluronic® F-68). In certain embodiments, the pH is 7.4. In certain embodiments, pharmaceutical formulation includes: 95 mM sodium chloride; 10 mM sodium phosphate (5 mM monobasic sodium phosphate and 5 mM dibasic sodium phosphate); 1.5 mM potassium phosphate; 7% w/v sucrose; and 0.001% w/v Poloxamer 188 copolymer.

In certain embodiments, the concentration of sodium chloride is 155 mM. In certain embodiments, the concentration of sodium phosphate is 2.7 mM. In certain embodiments, the concentration of potassium phosphate is 1.5 mM. In certain embodiments, the concentration of the sugar or sugar substitute is 5% w/v. In certain embodiments, the concentration of the copolymer is 0.001% w/v. In certain embodiments, the pharmaceutical formulation includes: 155 mM sodium chloride; 2.7 mM sodium phosphate; 1.5 mM potassium phosphate; 5% w/v sucrose; and 0.001% w/v Poloxamer 188 copolymer.

In certain embodiments, the pharmaceutical formulation includes at least one AAV particle, sodium chloride, potassium chloride, a sugar or sugar substitute and a copolymer. In certain embodiments, the pharmaceutical formulation includes Tris base to adjust pH.

In certain embodiments, the concentration of sodium chloride is 100 mM. In certain embodiments, the concentration of Tris is 10 mM. In certain embodiments, the concentration of potassium chloride is 1.5 mM. In certain embodiments, the concentration of the sugar or sugar substitute is 7% w/v. In certain embodiments, the concentration of the copolymer is 0.001% w/v. In certain embodiments, the sugar is sucrose. In certain embodiments, the copolymer is Poloxamer 188 (e.g., Pluronic® F-68). In certain embodiments, the pH is 8.

In certain embodiments, the one or more salts of the formulation includes sodium chloride. In certain embodiments, the concentration of sodium chloride in the formulation is between 80-220 mM or between 80-150 mM. In certain embodiments, the concentration of sodium chloride in the formulation is 75, 83, 92, 95, 98, 100, 107, 109, 118, 125, 127, 133, 142, 150, 155, 192, or 210 mM.

In certain embodiments, the one or more salts of the formulation includes potassium chloride. In certain embodiments, the concentration of potassium chloride in the formulation is between 0-10 mM, 1-2 mM, 1-3 mM, or 2-3 mM. In certain embodiments, the concentration of potassium chloride is 1.5 mM. In certain embodiments, the concentration of potassium chloride is 2.7 mM.

In certain embodiments, the one or more salts of the formulation includes potassium phosphate. In certain embodiments, the concentration of potassium phosphate in the formulation is between 0-10 mM or 1-3 mM. In certain embodiments, the concentration of potassium phosphate is 1.5 mM. In certain embodiments, the concentration of potassium phosphate is 2 mM.

In certain embodiments, the one or more salts of the formulation includes sodium phosphate. In certain embodiments, the concentration of sodium phosphate in the formulation is between 0-10 mM, 2-3 mM or 10-11 mM. In certain embodiments, the concentration of sodium phosphate is 2.7 mM. In certain embodiments, the concentration of sodium phosphate is 10 mM.

In certain embodiments, the concentration of sugar and/or sugar substitute in the formulation is between 1-10% w/v. In certain embodiments, the concentration of sugar and/or sugar substitute is 1%, 2%, 3%, 4%, 5%, 6%, 7%, 8%, 9%, or 10% w/v.

In certain embodiments, the one or more sugars or sugar substitutes include at least one disaccharide selected from sucrose, lactulose, lactose, maltose, trehalose, cellobiose, chitobiose, kojibiose, nigerose, isomaltose, β,β-trehalose, α,β-trehalose, sophorose, laminaribiose, gentiobiose, turanose, maltulose, palatinose, gentiobiulose, mannobiose, melibiose, melibiulose, rutinose, rutinulose, and xylobiose.

In certain embodiments, the least one sugar in the formulation includes sucrose and the concentration of sucrose is between 1-10% w/v. In certain embodiments, the concentration of sucrose in the formulation is 1%, 2%, 3%, 4%, 5%, 6%, 7%, 8%, 9%, or 10% w/v.

In certain embodiments, the least one sugar in the formulation includes trehalose and the concentration of trehalose is between 1-10% w/v. In certain embodiments, the concentration of trehalose in the formulation is 1%, 2%, 3%, 4%, 5%, 6%, 7%, 8%, 9%, or 10% w/v.

In certain embodiments, the least one sugar in the formulation includes sorbitol and the concentration of sorbitol is between 1-10% w/v. In certain embodiments, the concentration of sorbitol in the formulation is 1%, 2%, 3%, 4%, 5%, 6%, 7%, 8%, 9%, or 10% w/v.

In certain embodiments, the formulation includes one or more buffering agents. In certain embodiments, the formulation includes one or more buffering agents selected from Tris HCl, Tris base, sodium phosphate, potassium phosphate, histidine, boric acid, citric acid, glycine, HEPES (4-(2-hydroxyethyl)-1-piperazineethanesulfonic acid), and MOPS (3-(N-morpholino)propanesulfonic acid). In certain embodiments, the concentration of the buffering agent in the formulation is between 1-20 mM. In certain embodiments, the concentration of the buffering agent in the formulation is 10 mM.

In certain embodiments, the one or more buffering agents includes sodium phosphate and the formulation pH is from 7.2 to 7.6 at 5° C. In certain embodiments, the concentration of the sodium phosphate in the formulation is between 8-11 mM. In certain embodiments, the concentration of the sodium phosphate in the formulation is 10 mM.

In certain embodiments, the one or more buffering agents includes Tris base adjusted with hydrochloric acid. In certain embodiments, the formulation pH is from 7.3 to 8.2 at 5° C. In certain embodiments, the formulation pH is from 7.3 to 7.7 at 5° C. In certain embodiments, the formulation pH is from 7.8 to 8.2 at 5° C.

In certain embodiments, the formulation includes a copolymer surfactant. In certain embodiments, the concentration of the copolymer is between 0.00001%-1% w/v. In certain embodiments, the concentration of the copolymer is 0.00001%, 0.0001%, 0.001%, 0.01%, 0.1%, or 1% w/v. In one embodiment, the concentration is 0.001% w/v.

In certain embodiments, the copolymer is an ethylene oxide/propylene oxide copolymer. In certain embodiments, the concentration of the ethylene oxide/propylene oxide copolymer is between 0.00001%-1% w/v. In certain embodiments, the concentration of the ethylene oxide/propylene oxide copolymer is 0.00001%, 0.0001%, 0.001%, 0.01%, 0.1%, or 1% w/v. In certain embodiments, the copolymer is Poloxamer 188 (e.g., Pluronic® F-68). In certain embodiments, the concentration of the Poloxamer 188 copolymer is 0.01% w/v.

In certain embodiments, the concentration of AAV particle in the formulation described is less than 5×10¹³ vg/ml. In certain embodiments, the concentration of AAV particle in the formulation described is 2.7×10¹¹ vg/ml, 9×10¹¹ vg/ml, 1.2×10¹² vg/ml, 2.7×10¹² vg/ml, 4×10¹² vg/ml, 6×10¹² vg/ml, 7.9×10¹² vg/ml, 8×10¹² vg/ml, 1.8×10¹³ vg/ml, 2.7×10¹³ vg/ml, or 3.5×10¹³ vg/ml. In certain embodiments, the concentration of AAV particle in the formulation described is between 2.5-2.9×10¹³ vg/ml. In certain embodiments, the concentration of AAV particle in the formulation described is 2.7×10¹³ vg/ml.

In certain embodiments, the pharmaceutical formulation of the present disclosure includes an AAV particle which comprises an AAV vector genome and an AAV capsid. In certain embodiments, the AAV vector genome comprises the polynucleotide sequence of SEQ ID NO: 41.

In certain embodiments, the serotype of the AAV capsid is AAV1. In certain embodiments, the serotype of the AAV capsid is selected from: AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12. AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R. AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrb.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73. AAVrh.74, AAVrh8R, AAVrh8R A586R mutant, AAVrh8R R533A mutant, AAAV, BAAV, caprine AAV, bovine AAV, ovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrb.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, AAVF9/HSC9, PHP.B, PHP.A, G2B-26, G2B-13, TH1.1-32, TH1.1-35, or any of the modified serotypes of the present disclosure, or variants thereof.

The pharmaceutical gene therapy, e.g., AAV, formulations described herein may have an increased shelf-life, reduced aggregation, longer hold time for in-process pools, and/or increased concentration of AAV particles as compared to the same formulation without a sugar or sugar substitute.

The present disclosure presents methods of treating Huntington's Disease in a subject. In certain embodiments, the method includes administering to a subject a therapeutically effective amount of a pharmaceutical formulation of the present disclosure.

In certain embodiments, the pharmaceutical composition is administered via infusion into the putamen and thalamus of the subject. In certain embodiments, the pharmaceutical composition is administered via bilateral infusion into the putamen and thalamus of the subject. In certain embodiments, the pharmaceutical composition is administered using magnetic resonance imaging (MRI)-guided convection enhanced delivery (CED).

In certain embodiments, the volume of the pharmaceutical formulation administered to the putamen is no more than 1500 μL/hemisphere. In certain embodiments, the volume of the pharmaceutical formulation administered to the putamen is between 900-1500 μL/hemisphere. In certain embodiments, the dose administered to the putamen is between 8×10¹¹ to 4×10¹³ VG/hemisphere.

In certain embodiments, the volume of the pharmaceutical formulation administered to the thalamus is no more than 2500 μL/hemisphere. In certain embodiments, the volume of the pharmaceutical formulation administered to the thalamus is between 1300-2500 μL/hemisphere. In certain embodiments, the dose administered to the thalamus is between 3.5×10¹² to 6.8×10¹³ VG/hemisphere.

In certain embodiments, the total dose administered to the subject is between 8.6×10¹² to 2×10¹⁴ VG.

In certain embodiments, the administration of the pharmaceutical formulation to the subject inhibits or suppresses the expression of the Huntingtin (HTT) gene in the striatum of the subject. In certain embodiments, the expression of the HTT gene is inhibited or suppressed in the putamen. In certain embodiments, the expression of the HTT gene is inhibited or suppressed in one or more medium spiny neurons in the putamen. In certain embodiments, the HTT gene is inhibited or suppressed in one or more astrocytes in the putamen. In certain embodiments, the expression of the HTT gene in the putamen is reduced by at least 30%. In certain embodiments, the expression of the HTT gene in the putamen is reduced by 40-70%. In certain embodiments, the expression of the HTT gene in the putamen is reduced by 50-80%.

In certain embodiments, the expression of the HTT gene is inhibited or suppressed in the caudate. In certain embodiments, the HTT gene in the caudate is reduced by at least 30%. In certain embodiments, the HTT gene in the caudate is reduced by 40-70%. In certain embodiments, the HTT gene in the caudate is reduced by 50-85%.

In certain embodiments, the administration of the pharmaceutical formulation inhibits or suppresses the expression of the HT gene in the cerebral cortex of the subject. In certain embodiments, the expression of the HTT gene is inhibited or suppressed in the primary motor and somatosensory cortex. In certain embodiments, the expression of the HTT gene is inhibited or suppressed in the pyramidal neurons of primary motor and somatosensory cortex. In certain embodiments, the expression of the HTT gene in the cerebral cortex is reduced by at least 20%. In certain embodiments, the expression of the HTT gene in the cerebral cortex is reduced by 30-70%.

In certain embodiments, the administration of the pharmaceutical composition inhibits or suppresses the expression of the HTT gene in the thalamus of the subject. In certain embodiments, the expression of the HTT gene in the thalamus is reduced by at least 30%. In certain embodiments, the expression of the HTT gene in the thalamus is reduced by 40-80%.

### BRIEF DESCRIPTION OF THE FIGURES

The foregoing and other objects, features and advantages will be apparent from the following description of particular embodiments of the present disclosure, as illustrated in the accompanying figures. The figures are not necessarily to scale or comprehensive, with emphasis instead being placed upon illustrating the principles of various embodiments of the present disclosure.

FIG. 1 shows a schematic for one embodiment of a system, and a flow diagram for one embodiment of a process, for producing baculovirus infected insect cells (BIICs) using Viral Production Cells (VPC) and plasmid constructs.

FIG. 2 shows a schematic for one embodiment of a system, and a flow diagram for one embodiment of a process, for producing AAV Particles using Viral Production Cells (VPC) and baculovirus infected insect cells (BIICs).

FIG. 3 shows schematic for one embodiment of a system, and a flow diagram for one embodiment of a process, for producing a Drug Substance by processing, clarifying and purifying a bulk harvest of AAV particles and Viral Production Cells.

FIG. 4A shows Log₁₀ reduction values for Baculovirus (BACV) viral contaminants (TCID50) using an anion exchange chromatography system in flow-through mode, according to certain embodiments of the present disclosure.

FIG. 4B shows Log₁₀ reduction values for Vesicular Stomatitis Virus (VSV) viral contaminants (TCID50) using an anion exchange chromatography system in flow-through mode, according to certain embodiments of the present disclosure.

FIG. 4C shows Log₁₀ reduction values for Human Adenovirus Type 5 (Ad5) viral contaminants (TCID50) using an anion exchange chromatography system in flow-through mode, according to certain embodiments of the present disclosure.

FIG. 4D shows Log₁₀ reduction values for Reovirus Type 3 (Reo3) viral contaminants (TCID50) using an anion exchange chromatography system in flow-through mode, according to certain embodiments of the present disclosure.

FIGS. 5A-5C are panels of graphs showing HTT mRNA knockdown and vector genome levels in tissue punches collected from non-human primate (NHP) putamen.

FIGS. 6A-5C are panels of graphs showing HTT mRNA knockdown and vector genome levels in tissue punches collected from NHP caudate.

FIGS. 7A-7C are panels of graphs showing HTT mRNA knockdown and vector genome levels in tissue punches collected from NHP motor cortex (mCTX).

FIGS. 8A-8C are panels of graphs showing HTT mRNA knockdown and vector genome levels in tissue punches collected from NHP somatosensory cortex (ssCTX).

FIGS. 9A-9C are panels of graphs showing HTT mRNA knockdown and vector genome levels in tissue punches collected from NHP temporal cortex (tCTX).

FIG. 10A and FIG. 10B are graphs showing HTT mRNA knockdown and vector genome levels, respectively, in laser captured cortical pyramidal neurons from NHP cortex.

FIG. 11A shows a correlation curve of HTT mRNA knockdown versus vector genome levels in tissue punches taken from the putamen.

FIG. 11B shows a correlation curve of vector genome versus AAV1-VOYHT1 miRNA levels in tissue punches taken from the putamen.

FIG. 11C shows a correlation curve of AAV1-VOYHT1 miRNA versus HTT mRNA levels in tissue punches taken from the putamen.

FIG. 12A shows a correlation curve of HTT mRNA knockdown versus vector genome levels in tissue punches taken from the caudate.

FIG. 12B shows a correlation curve of vector genome versus AAV1-VOYHT1 miRNA levels in tissue punches taken from the caudate.

FIG. 12C shows a correlation curve of AAV1-VOYHT1 miRNA versus HTT mRNA levels in tissue punches taken from the caudate.

FIG. 13 shows a correlation curve of HTT mRNA knockdown versus vector genome levels in tissue punches taken from the thalamus.

### DETAILED DESCRIPTION

### I. Adeno-Associated Viruses (AAVs)

### Overview

Adeno-associated viruses (AAV) are small non-enveloped icosahedral capsid viruses of the Parvoviridae family characterized by a single stranded DNA viral genome. Parvoviridae family viruses consist of two subfamilies: Parvovirinae, which infect vertebrates, and Densovirinae, which infect invertebrates. The Parvoviridae family includes the Dependovirus genus which includes AAV, capable of replication in vertebrate hosts including, but not limited to, human, primate, bovine, canine, equine, and ovine species.

The parvoviruses and other members of the Parvoviridae family are generally described in Kenneth I. Berns, “Parvoviridae: The Viruses and Their Replication,” Chapter 69 in Fields Virology (3d Ed. 1996), the contents of which are incorporated by reference in their entirety.

AAV have proven to be useful as a biological tool due to their relatively simple structure, their ability to infect a wide range of cells (including quiescent and dividing cells) without integration into the host genome and without replicating, and their relatively benign immunogenic profile. The genome of the virus may be manipulated to contain a minimum of components for the assembly of a functional recombinant virus, or viral particle, which is loaded with or engineered to target a particular tissue and express or deliver a desired payload.

### AAV Viral Genomes

The wild-type AAV viral genome is a linear, single-stranded DNA (ssDNA) molecule approximately 5,000 nucleotides (nt) in length. Inverted terminal repeats (ITRs) traditionally flank the viral genome at both the 5′ and the 3′ end, providing origins of replication for the viral genome. While not wishing to be bound by theory, an AAV viral genome typically includes two ITR sequences. These ITRs have a characteristic T-shaped hairpin structure defined by a self-complementary region (145nt in wild-type AAV) at the 5′ and 3′ ends of the ssDNA which form an energetically stable double stranded region. The double stranded hairpin structures include multiple functions including, but not limited to, acting as an origin for DNA replication by functioning as primers for the endogenous DNA polymerase complex of the host viral replication cell.

The wild-type AAV viral genome further includes nucleotide sequences for two open reading frames, one for the four non-structural Rep proteins (Rep78, Rep68, Rep52, Rep40, encoded by Rep genes) and one for the three capsid, or structural, proteins (VP1, VP2, VP3, encoded by capsid genes or Cap genes). The Rep proteins are important for replication and packaging, while the capsid proteins are assembled to create the protein shell of the AAV, or AAV capsid. Alternative splicing and alternate initiation codons and promoters result in the generation of four different Rep proteins from a single open reading frame and the generation of three capsid proteins from a single open reading frame. Though it varies by AAV serotype, as a non-limiting example, for AAV9/hu.14 (SEQ ID NO: 123 of U.S. Pat. No. 7,906,111, the contents of which are herein incorporated by reference in their entirety) VP1 refers to amino acids 1-736, VP2 refers to amino acids 138-736, and VP3 refers to amino acids 203-736. In other words. VP1 is the full-length capsid sequence, while VP2 and VP3 are shorter components of the whole. As a result, changes in the sequence in the VP3 region, are also changes to VP1 and VP2, however, the percent difference as compared to the parent sequence will be greatest for VP3 since it is the shortest sequence of the three. Though described here in relation to the amino acid sequence, the nucleic acid sequence encoding these proteins can be similarly described. Together, the three capsid proteins assemble to create the AAV capsid protein. While not wishing to be bound by theory, the AAV capsid protein typically includes a molar ratio of 1:1:10 of VP1:VP2:VP3. As used herein, an “AAV serotype” is defined primarily by the AAV capsid. In some instances, the ITRs are also specifically described by the AAV serotype (e.g., AAV2/9).

For use as a biological tool, the wild-type AAV viral genome can be modified to replace the rep/cap sequences with a nucleic acid sequence including a payload region with at least one ITR region. Typically, in recombinant AAV viral genomes there are two ITR regions. The rep/cap sequences can be provided in trans during production to generate AAV particles.

In addition to the encoded heterologous payload, AAV vectors may include the viral genome, in whole or in part, of any naturally occurring and/or recombinant AAV serotype nucleotide sequence or variant. AAV variants may have sequences of significant homology at the nucleic acid (genome or capsid) and amino acid levels (capsids), to produce constructs which are generally physical and functional equivalents, replicate by similar mechanisms, and assemble by similar mechanisms. See Chiorini et al., J. Vir. 71: 6823-33(1997); Srivastava et al., J. Vir. 45:555-64 (1983); Chiorini et al., J. Vir. 73:1309-1319 (1999); Rutledge et al., J. Vir. 72:309-319 (1998); and Wu et al., J. Vir. 74: 8635-47 (2000), the contents of each of which are incorporated herein by reference in their entirety.

In certain embodiments, AAV particles, viral genomes and/or payloads of the present disclosure, and the methods of their use, may be as described in WO2017189963, the contents of which are herein incorporated by reference in their entirety.

AAV particles of the present disclosure may be formulated in any of the gene therapy formulations of the disclosure including any variations of such formulations apparent to those skilled in the art. The reference to “AAV particles”, “AAV particle formulations” and “formulated AAV particles” in the present application refers to the AAV particles which may be formulated and those which are formulated without limiting either.

In certain embodiments, AAV particles of the present disclosure are recombinant AAV (rAAV) viral particles which are replication defective, lacking sequences encoding functional Rep and Cap proteins within their viral genome. These defective AAV particles may lack most or all parental coding sequences and essentially carry only one or two AAV ITR sequences and the nucleic acid of interest (i.e., payload) for delivery to a cell, a tissue, an organ or an organism.

In certain embodiments, the viral genome of the AAV particles of the present disclosure includes at least one control element which provides for the replication, transcription and translation of a coding sequence encoded therein. Not all of the control elements need always be present as long as the coding sequence is capable of being replicated, transcribed and/or translated in an appropriate host cell. Non-limiting examples of expression control elements include sequences for transcription initiation and/or termination, promoter and/or enhancer sequences, efficient RNA processing signals such as splicing and polyadenylation signals, sequences that stabilize cytoplasmic mRNA, sequences that enhance translation efficacy (e.g., Kozak consensus sequence), sequences that enhance protein stability, and/or sequences that enhance protein processing and/or secretion.

According to the present disclosure, AAV particles for use in therapeutics and/or diagnostics include a virus that has been distilled or reduced to the minimum components necessary for transduction of a nucleic acid payload or cargo of interest. In this manner, AAV particles are engineered as vehicles for specific delivery while lacking the deleterious replication and/or integration features found in wild-type viruses.

AAV particles of the present disclosure may be produced recombinantly and may be based on adeno-associated virus (AAV) parent or reference sequences. As used herein, a “vector” is any molecule or moiety which transports, transduces or otherwise acts as a carrier of a heterologous molecule such as the nucleic acids described herein.

In addition to single stranded AAV viral genomes (e.g., ssAAVs), the present disclosure also provides for self-complementary AAV (scAAVs) viral genomes. scAAV vector genomes contain DNA strands which anneal together to form double stranded DNA. By skipping second strand synthesis, scAAVs allow for rapid expression in the cell.

In certain embodiments, the AAV viral genome of the present disclosure is a scAAV. In certain embodiments, the AAV viral genome of the present disclosure is a ssAAV.

Methods for producing and/or modifying AAV particles are disclosed in the art, such as pseudotyped AAV particles (PCT Patent Publication Nos. WO200028004; WO200123001; WO2004112727; WO 2005005610 and WO 2005072364, the content of each of which is incorporated herein by reference in its entirety).

AAV particles may be modified to enhance the efficiency of delivery. Such modified AAV particles can be packaged efficiently and be used to successfully infect the target cells at high frequency and with minimal toxicity. In certain embodiments the capsids of the AAV particles are engineered according to the methods described in US Publication Number US 20130195801, the contents of which are incorporated herein by reference in their entirety.

In certain embodiments, the AAV particles including a payload region encoding a polypeptide or protein of the present disclosure, and may be introduced into mammalian cells.

### AAV Serotypes

AAV particles of the present disclosure may include or be derived from any natural or recombinant AAV serotype. According to the present disclosure, the AAV particles may utilize or be based on a serotype or include a peptide selected from any of the following: AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b. AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrb.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrb.2R. AAVrh.8, AAVrh.8R. AAVrb.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh46, AAVrh.48, AAVrh.48.1, AAVrh.48.1.2, AAVrh.48.2, AAVrb.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh 64R2, AAVrh.67, AAVrh.73, AAVrh.74, AAVrh8R, AAVrh8R A586R mutant, AAVrh8R R533A mutant, AAAV. BAAV, caprine AAV, bovine AAV, ovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrb.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4. AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17. AAVF2/HSC2, AAVF3/HSC3, AAVF4/HSC4, AAVF5/HSC5, AAVF6/HSC6, AAVF7/HSC7, AAVF8/HSC8, AAVF9/HSC9, AAVrh20, AAVrh32/33, AAVrh39, AAVrh46, AAVrh73, AAVrh74, AAVhu.26, VOY101, VOY201, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3 (G2A3), AAVG2B4 (G2B4), AAVG2B5 (G2B5), PHP.S, or variants or derivatives thereof.

In some embodiments, the AAV may be a serotype selected from any of those found in Table 1.

In some embodiments, the AAV may comprise a sequence, fragment or variant thereof, of the sequences in Table 1.

In some embodiments, the AAV may be encoded by a sequence, fragment or variant as described in Table 1.

TABLE 1
AAV Serotypes

| | SEQ | |
|---|---|---|
| | ID | |
| Serotype | NO | Reference Information |
| AAV1 (nt) | 1 | US20030138772 SEQ ID NO: 6 |
| AAV1 (aa) | 2 | US20160017295 SEQ ID NO: 1, US20030138772 SEQ ID NO: 64, |
| | | US20150159173 SEQ ID NO: 27, US20150315612 SEQ ID NO: 219, |
| | | U.S. Pat. No. 7,198,951 SEQ ID NO: 5 |
| AAV2 (nt) | 3 | US20150159173 SEQ ID NO: 7, US20150315612 SEQ ID NO: 211 |
| AAV2 (aa) | 4 | US20030138772 SEQ ID NO: 70, US20150159173 SEQ ID NO: 23, |
| | | US20150315612 SEQ ID NO: 221, US20160017295 SEQ ID NO: 2, |
| | | U.S. Pat. No. 6,156,303 SEQ ID NO: 4, U.S. Pat. No. 7,198,951, |
| | | SEQ ID NO: 4, WO2015121501 SEQ ID NO: 1 |
| AAV3 (nt) | 5 | US20030138772 SEQ ID NO: 8 |
| AAV3 (aa) | 6 | US20030138772 SEQ ID NO: 71, US20150159173 SEQ ID NO: 28, |
| | | US20160017295 SEQ ID NO: 3, U.S. Pat. No. 7,198,951 SEQ ID NO: 6 |
| AAV4 (nt) | 7 | US20140348794 SEQ ID NO: 1 |
| AAV4 (nt) | 8 | WO2016065001 SEQ ID NO: 49 |
| AAV4 (aa) | 9 | US20030138772 SEQ ID NO: 63, US20160017295 SEQ ID NO: 4, |
| | | US20140348794 SEQ ID NO: 4 |
| AAV5 (nt) | 10 | U.S. Pat. No. 7,427,396 SEQ ID NO: 1 |
| AAV5 (aa) | 11 | US20160017295 SEQ ID NO5, U.S. Pat. No. 7,427,396 SEQ ID NO: 2, |
| | | US20150315612 SEQ ID NO: 216 |
| AAV6 (nt) | 12 | U.S. Pat. No. 6,156,303 SEQ ID NO: 2 |
| AAV6 (nt) | 13 | US20150315612 SEQ ID NO: 203 |
| AAV6 (aa) | 14 | US20030138772 SEQ ID NO: 65, US20150159173 SEQ ID NO: 29, |
| | | US20160017295 SEQ ID NO: 6, U.S. Pat. No. 6,156,303 SEQ ID NO: 7 |
| AAV7 (nt) | 15 | US20150159173 SEQ ID NO: 14 |
| AAV7 (nt) | 16 | US20030138772 SEQ ID NO: 1, US20150315612 SEQ ID NO: 180 |
| AAV7 (aa) | 17 | US20030138772 SEQ ID NO: 2, US20150159173 SEQ D NO: 30, |
| | | US20150315612 SEQ ID NO: 181, US20160017295 SEQ ID NO: 7 |
| AAV8 (nt) | 18 | US20030138772 SEQ ID NO: 4, US20150315612 SEQ ID NO: 182 |
| AAV8 (nt) | 19 | US20150159173 SEQ ID NO: 15 |
| AAV8 (aa) | 20 | US20030138772 SEQ ID NO: 95, US20140359799 SEQ ID NO: 1, |
| | | US20150159173 SEQ ID NO: 31, US20160017295 SEQ ID NO: 8, |
| | | U.S. Pat. No. 7,198,951 SEQ ID NO: 7, US20150315612 SEQ ID NO: 223 |
| AAV9/hu.14 (nt) | 21 | SEQ ID NO: 3; U.S. Pat. No. 7,906,111 |
| AAV9/hu.14 (aa) | 22 | SEQ ID NO: 123; U.S. Pat. No.7,906,111 |
| AAV PHP.B (nt) | 23 | SEQ ID NO: 9; WO2015038958 |
| AAV PHP.B (aa) | 24 | SEQ ID NO: 8; WO201503895S |
| (K449R) | | |
| AAVG2B-13 | 25 | SEQ ID NO: 12; WO2015038958 |
| AAVTH1.1-32 | 26 | SEQ ID NO: 14; WO2015038958 |
| AAVTH1.1-35 | 27 | SEQ ID NO: 15; WO2015038958 |
| PHP.N/PHP.B- | 28 | SEQ ID NO: 46; WO2017100671 |
| DGT | | |
| PHP.S/G2A12 | 29 | SEQ ID NO: 47; WO2017100671 |
| AAV9/hu.14 | 30 | SEQ ID NO: 45; WO2017100671 |
| K449R | | |
| AAVrh10 (nt) | 31 | US2003138772 SEQ ID NO: 59 |
| (referred to as | | |
| clone 44.2) | | |
| AAVrh10 (aa) | 32 | US20030138772 SEQ ID NO: 81 |
| (referred to as | | |
| clone 44.2) | | |
| AAV-DJ (nt) | 33 | US20140359799 SEQ ID NO: 3, U.S. Pat. No. 7,588,772 SEQ ID NO: 2 |
| AAV-DJ (aa) | 34 | US20140359799 SEQ ID NO: 2, U.S. Pat. No. 7,588,772 SEQ ID NO: 1 |
| AAV-DJ8 | 35 | U.S. Pat. No. 7,588,772; Grimm et al 2008 |
| (2 mutations) | | |
| AAV-DJ8 | 36 | U.S. Pat. No. 7,588,772; Grimm et al 2008 |
| (3 mutations) | | |
| rh74 (nt) | 37 | US9434928B2 SEQ ID NO: 1; US2015023924A1 SEQ ID NO: 2 |
| rh74 (aa) | 38 | US9434928B2 SEQ ID NO: 2; US2015023924A1 SEQ ID NO: 1 |
| AAV10 (aa) | 39 | WO2015121501 SEQ ID NO: 9 |
| AAV10 (aa) | 40 | WO2015121501 SEQ ID NO: 8 |

Each of the patents, applications and/or publications listed in Table 1 are hereby incorporated by reference in their entirety.

In some embodiments, the serotype may be AAVDJ (or AAV-DJ) or a variant thereof, such as AAVDJ8 (or AAV-DJ8), as described by Grimm et al. (Journal of Virology 82(12): 5887-5911 (2008), herein incorporated by reference in its entirety). The amino acid sequence of AAVDJ8 may comprise two or more mutations in order to remove the heparin binding domain (HBD). As a non-limiting example, the AAV-DJ sequence described as SEQ ID NO: 1 in U.S. Pat. No. 7,588,772, the contents of which are herein incorporated by reference in their entirety, may include two mutations: (1) R587Q where arginine (R; Arg) at amino acid 587 is changed to glutamine (Q; Gin) and (2) R590T where arginine (R; Arg) at amino acid 590 is changed to threonine (T; Thr). As another non-limiting example, may include three mutations: (1) K406R where lysine (K; Lys) at amino acid 406 is changed to arginine (R; Arg), (2) R587Q where arginine (R; Arg) at amino acid 587 is changed to glutamine (Q; Gln) and (3) R590T where arginine (R; Arg) at amino acid 590 is changed to threonine (T; Thr).

In some embodiments, the AAV serotype may be, or have, a modification as described in United States Publication No. US 20160361439, the contents of which are herein incorporated by reference in their entirety, such as but not limited to, Y252F, Y272F, Y444F, Y500F, Y700F, Y704F, Y730F, Y275F, Y281F, Y508F, Y576F, Y612G, Y673F, and Y720F of the wild-type AAV1, AAV2, AAV3, AAV4, AAV5, AAV6, AAV7, AAV8, AAV9, AAV10, AAV11, AAV12, and hybrids thereof.

In some embodiments, the AAV serotype may be, or have, a mutation as described in U.S. Pat. No. 9,546,112, the contents of which are herein incorporated by reference in their entirety, such as, but not limited to, at least two, but not all the F129L, D418E, K531E, L584F, V598A and H642N mutations in the sequence of AAV6 (SEQ ID NO:4 of U.S. Pat. No. 9,546,112), AAV1 (SEQ ID NO:6 of U.S. Pat. No. 9,546,112), AAV2, AAV3, AAV4, AAV5, AAV7, AAV9, AAV10 or AAV11 or derivatives thereof. In yet another embodiment, the AAV serotype may be, or have, an AAV6 sequence comprising the K531E mutation (SEQ ID NO:5 of U.S. Pat. No. 9,546,112).

In some embodiments, the AAV serotype may be, or have, a mutation in the AAV1 sequence, as described in in United States Publication No. US 20130224836, the contents of which are herein incorporated by reference in their entirety, such as, but not limited to, at least one of the surface-exposed tyrosine residues, preferably, at positions 252, 273, 445, 701, 705 and 731 of AAV1 (SEQ ID NO: 2 of US 20130224836) substituted with another amino acid, preferably with a phenylalanine residue. In some embodiments, the AAV serotype may be, or have, a mutation in the AAV9 sequence, such as, but not limited to, at least one of the surface-exposed tyrosine residues, preferably, at positions 252, 272, 444, 500, 700, 704 and 730 of AAV2 (SEQ ID NO: 4 of US 20130224836) substituted with another amino acid, preferably with a phenylalanine residue. In some embodiments, the tyrosine residue at position 446 of AAV9 (SEQ ID NO: 6 US 20130224836) is substituted with a phenylalanine residue.

In some embodiments, the AAV serotype may be, or have, a mutation in the AAV9 sequence as described by N Pulicherla et al. (Molecular Therapy 19(6):1070-1078 (2011), herein incorporated by reference in its entirety), such as but not limited to, AAV9.9, AAV9.11, AAV9.13, AAV9.16, AAV9.24, AAV9.45, AAV9.47, AAV9.61, AAV9.68, AAV9.84.

In some embodiments, the serotype may be AAV2 or a variant thereof, as described in International Publication No. WO2016130589, herein incorporated by reference in its entirety. The amino acid sequence of AAV2 may comprise N587A, E548A, or N708A mutations. In some embodiments, the amino acid sequence of any AAV may comprise a V708K mutation.

In some embodiments, the AAV serotype may be, or may have a sequence as described in United States Publication No. US 20160369298, the contents of which are herein incorporated by reference in their entirety, such as, but not limited to, site-specific mutated capsid protein of AAV2 (SEQ ID NO: 97 of US 20160369298) or variants thereof, wherein the specific site is at least one site selected from sites R447, G453, S578, N587, N587+1, S662 of VP1 or fragment thereof.

In some embodiments, the AAV serotype may be modified as described in the United States Publication US 20170145405 the contents of which are herein incorporated by reference in their entirety. AAV serotypes may include, modified AAV2 (e.g., modifications at Y444F, Y500F, Y730F and/or S662V), modified AAV3 (e.g., modifications at Y705F, Y731F and/or T492V), and modified AAV6 (e.g., modifications at S663V and/or T492V).

In some embodiments, the AAV capsid serotype selection or use may be from a variety of species. In some embodiments, the AAV may be an avian AAV (AAAV). The AAAV serotype may be, or have, a sequence as described in U.S. Pat. No. 9,238,800, the contents of which are herein incorporated by reference in their entirety, such as, but not limited to, AAAV (SEQ ID NO: 1, 2, 4, 6, 8, 10, 12, and 14 of U.S. Pat. No. 9,238,800), or variants thereof.

In some embodiments, the AAV may be a bovine AAV (BAAV). The BAAV serotype may be, or have, a sequence as described in U.S. Pat. No. 9,193,769, the contents of which are herein incorporated by reference in their entirety, such as, but not limited to, BAAV (SEQ ID NO: 1 and 6 of U.S. Pat. No. 9,193,769), or variants thereof. The BAAV serotype may be or have a sequence as described in U.S. Pat. No. 7,427,396, the contents of which are herein incorporated by reference in their entirety, such as, but not limited to, BAAV (SEQ ID NO: 5 and 6 of U.S. Pat. No. 7,427,396), or variants thereof.

In some embodiments, the AAV may be a caprine AAV. The caprine AAV serotype may be, or have, a sequence as described in U.S. Pat. No. 7,427,396, the contents of which are herein incorporated by reference in their entirety, such as, but not limited to, caprine AAV (SEQ ID NO: 3 of U.S. Pat. No. 7,427,396), or variants thereof.

In other embodiments the AAV may be engineered as a hybrid AAV from two or more parental serotypes. In some embodiments, the AAV may be AAV2G9 which comprises sequences from AAV2 and AAV9. The AAV2G9 AAV serotype may be, or have, a sequence as described in United States Patent Publication No. US20160017005, the contents of which are herein incorporated by reference in its entirety.

In certain embodiments, the AAV may be a serotype generated by the AAV9 capsid library with mutations in amino acids 390-627 (VP1 numbering) as described by Pulicherla et al. (Molecular Therapy 19(6):1070-1078 (2011), the contents of which are herein incorporated by reference in their entirety. The serotype and corresponding nucleotide and amino acid substitutions may be, but is not limited to, AAV9.1 (G1594C; D532H), AAV6.2 (T1418A and T1436X; V473D and 1479K), AAV9.3 (T1238A; F413Y), AAV9.4 (T1250C and A1617T; F417S), AAV9.5 (A1235G, A1314T, A1642G, C1760T; Q412R, T548A, A587V), AAV9.6 (T1231A; F4111), AAV9.9 (G1203A, G1785T; W595C), AAV9.10 (A1500G, T1676C; M559T), AAV9.11 (A1425T, A1702C, A1769T; T568P, Q590L), AAV9.13 (A1369C, A1720T; N457H, T574S), AAV9.14 (T1340A, T1362C, T1560C, G1713A; L447H), AAV9.16 (A1775T; Q592L), AAV9.24 (T1507C, T1521G; W503R), AAV9.26 (A1337G, A1769C; Y446C, Q590P), AAV9.33 (A1667C; D556A), AAV9.34 (A1534G. C1794T; N512D), AAV9.35 (A1289T, T1450A, C1494T, A1515T, C1794A, G1816A; Q430L, Y484N, N98K, V606I), AAV9.40 (A1694T, E565V), AAV9.41 (A1348T, T1362C; T450S), AAV9.44 (A1684C, A1701T, A1737G; N562H, K567N), AAV9.45 (A1492T, C1804T; N498Y, L602F), AAV9.46 (G1441C, T1525C, T1549G; G481R, W509R, L517V), 9.47 (G1241A, G1358A, A1669G, C1745T; S414N, G453D, K557E, T5821), AAV9.48 (C1445T, A1736T; P482L, Q579L), AAV9.50 (A1638T, C1683T, T1805A; Q546H, L602H), AAV9.53 (G1301A, A1405C, C1664T, G1811 T; R134Q, S469R, A555V, G604V), AAV9.54 (C1531A, T1609A; L5111, L537M), AAV9.55 (T1605A; F535L), AAV9.58 (C1475T, C1579A; T4921, H527N), AAV.59 (T1336C; Y446H), AAV9.61 (A1493T; N498I), AAV9.64 (C1531A, A1617T; L511 I), AAV9.65 (C1335T, T1530C, C1568A; A523D), AAV9.68 (C1510A; P504T), AAV9.80 (G1441A; G481R), AAV9.83 (C1402A, A1500T; P468T, E500D), AAV9.87 (T1464C, T1468C; S490P), AAV9.90 (A1196T; Y399F), AAV9.91 (T1316G, A1583T, C1782G, T1806C; L439R, K5281), AAV9.93 (A1273G, A1421G, A1638C, C1712T, G1732A, A1744T, A1832T; S425G. Q474R, Q546H, P571L, G578R, T582S, D611V), AAV9.94 (A1675T; M559L) and AAV9.95 (T1605A; F535L).

In any of the DNA and RNA sequences referenced and/or described herein, the single letter symbol has the following description: A for adenine; C for cytosine; G for guanine; T for thymine; U for Uracil; W for weak bases such as adenine or thymine; S for strong nucleotides such as cytosine and guanine; M for amino nucleotides such as adenine and cytosine; K for keto nucleotides such as guanine and thymine; R for purines adenine and guanine; Y for pyrimidine cytosine and thymine; B for any base that is not A (e.g., cytosine, guanine, and thymine); D for any base that is not C (e.g., adenine, guanine, and thymine); H for any base that is not G (e.g., adenine, cytosine, and thymine); V for any base that is not T (e.g., adenine, cytosine, and guanine); N for any nucleotide (which is not a gap); and Z is for zero.

In any of the amino acid sequences referenced and/or described herein, the single letter symbol has the following description: G (Gly) for Glycine; A (Ala) for Alanine; L (Leu) for Leucine; M (Met) for Methionine; F (Phe) for Phenylalanine; W (Trp) for Tryptophan; K (Lys) for Lysine; Q (Gln) for Glutamine; E (Glu) for Glutamic Acid; S (Ser) for Serine; P (Pro) for Proline; V (Val) for Valine; I (Ile) for Isoleucine; C (Cys) for Cysteine; Y (Tyr) for Tyrosine; H (His) for Histidine; R (Arg) for Arginine; N (Asn) for Asparagine; D (Asp) for Aspartic Acid; T (Thr) for Threonine; B (Asx) for Aspartic acid or Asparagine; J (Xle) for Leucine or Isoleucine; O (Pyl) for Pyrrolysine; U (See) for Selenocysteine; X (Xaa) for any amino acid; and Z (Glx) for Glutamine or Glutamic acid.

In certain embodiments, the AAV serotype may be, or may include a sequence, insert, modification or mutation as described in Patent Publications WO2015038958, WO2017100671, WO2016134375, WO2017083722, WO2017015102, WO2017058892, WO2017066764, U.S. Pat. Nos. 9,624,274, 9,475,845, US20160369298, US20170145405, the contents of which are herein incorporated by reference in their entirety.

In certain embodiments, the AAV may be a serotype generated by Cre-recombination-based AAV targeted evolution (CREATE) as described by Deverman et al., (Nature Biotechnology 34(2):204-209 (2016)), the contents of which are herein incorporated by reference in their entirety. In certain embodiments, the AAV serotype may be as described in Jackson et al (Frontiers in Molecular Neuroscience 9:154 (2016)), the contents of which are herein incorporated by reference in their entirety. In some embodiments, AAV serotypes generated in this manner have improved CNS transduction and/or neuronal and astrocytic tropism, as compared to other AAV serotypes. As non-limiting examples, the AAV serotype may be PHP.B, PHP.B2, PHP.B3, PHP.A, G2A12, G2A15. In some embodiments, these AAV serotypes may be AAV9 derivatives with a 7-amino acid insert between amino acids 588-589.

In certain embodiments, the AAV serotype is selected for use due to its tropism for cells of the central nervous system. In certain embodiments, the cells of the central nervous system are neurons. In another embodiment, the cells of the central nervous system are astrocytes.

In certain embodiments, the AAV serotype is selected for use due to its tropism for cells of the muscle(s).

In some embodiments, the AAV serotype is PHP.B or AAV9. In some embodiments, the AAV serotype is paired with a synapsin promoter to enhance neuronal transduction, as compared to when more ubiquitous promoters are used (e.g., CBA or CMV).

In certain embodiments, the initiation codon for translation of the AAV VP1 capsid protein may be CTG, TTG, or GTG as described in U.S. Pat. No. 8,163,543, the contents of which are herein incorporated by reference in its entirety.

The present disclosure refers to structural capsid proteins (including VP1, VP2 and VP3) which are encoded by capsid (Cap) genes. These capsid proteins form an outer protein structural shell (i.e. capsid) of a viral vector such as AAV. VP capsid proteins synthesized from Cap polynucleotides generally include a methionine as the first amino acid in the peptide sequence (Met1), which is associated with the start codon (AUG or ATG) in the corresponding Cap nucleotide sequence. However, it is common for a first-methionine (Met1) residue or generally any first amino acid (AA1) to be cleaved off after or during polypeptide synthesis by protein processing enzymes such as Met-aminopeptidases. This “Met/AA-clipping” process often correlates with a corresponding acetylation of the second amino acid in the polypeptide sequence (e.g., alanine, valine, serine, threonine, etc.). Met-clipping commonly occurs with VP1 and VP3 capsid proteins but can also occur with VP2 capsid proteins.

Where the Met/AA-clipping is incomplete, a mixture of one or more (one, two or three) VP capsid proteins including the viral capsid may be produced, some of which may include a Met1/AA1 amino acid (Met+/AA+) and some of which may lack a Met1/AA1 amino acid as a result of Met/AA-clipping (Met−/AA−). For further discussion regarding Met/AA-clipping in capsid proteins, see Jin, et al. Direct Liquid Chromatography/Mass Spectrometry Analysis for Complete Characterization of Recombinant Adeno-Associated Virus Capsid Proteins. Hum Gene Ther Methods. 2017 Oct. 28(5):255-267; Hwang, et al. N-Terminal Acetylation of Cellular Proteins Creates Specific Degradation Signals. Science. 2010 Feb. 19. 327(5968): 973-977; the contents of which are each incorporated herein by reference in their entirety.

According to the present disclosure, references to capsid proteins is not limited to either clipped (Met−/AA−) or unclipped (Met+/AA+) and may, in context, refer to independent capsid proteins, viral capsids included of a mixture of capsid proteins, and/or polynucleotide sequences (or fragments thereof) which encode, describe, produce or result in capsid proteins of the present disclosure. A direct reference to a “capsid protein” or “capsid polypeptide” (such as VP1, VP2 or VP2) may also include VP capsid proteins which include a Met1/AA1 amino acid (Met+/AA+) as well as corresponding VP capsid proteins which lack the Met1/AA1 amino acid as a result of Met/AA-clipping (Met−/AA−).

Further according to the present disclosure, a reference to a specific SEQ ID NO: (whether a protein or nucleic acid) which includes or encodes, respectively, one or more capsid proteins which include a Met1/AA1 amino acid (Met+/AA+) should be understood to teach the VP capsid proteins which lack the Met1/AA1 amino acid as upon review of the sequence, it is readily apparent any sequence which merely lacks the first listed amino acid (whether or not Met1/AA1).

As a non-limiting example, reference to a VP1 polypeptide sequence which is 736 amino acids in length and which includes a “Met1” amino acid (Met+) encoded by the AUG/ATG start codon may also be understood to teach a VP1 polypeptide sequence which is 735 amino acids in length and which does not include the “Met1” amino acid (Met−) of the 736 amino acid Met+ sequence. As a second non-limiting example, reference to a VP1 polypeptide sequence which is 736 amino acids in length and which includes an “AA1” amino acid (AA1+) encoded by any NNN initiator codon may also be understood to teach a VP1 polypeptide sequence which is 735 amino acids in length and which does not include the “AA1” amino acid (AA1−) of the 736 amino acid AA1+ sequence.

References to viral capsids formed from VP capsid proteins (such as reference to specific AAV capsid serotypes), can incorporate VP capsid proteins which include a Met1/AA1 amino acid (Met+/AA1+), corresponding VP capsid proteins which lack the Met1/AA1 amino acid as a result of Met/AA1-clipping (Met−/AA1−), and combinations thereof (Met+/AA1+ and Met−/AA1−).

As a non-limiting example, an AAV capsid serotype can include VP1 (Met+/AA1+), VP1 (Met−/AA1−), or a combination of VP1 (Met+/AA1+) and VP1 (Met−/AA1−). An AAV capsid serotype can also include VP3 (Met+/AA1+), VP3 (Met−/AA1−), or a combination of VP3 (Met+/AA1+) and VP3 (Met−/AA1−); and can also include similar optional combinations of VP2 (Met+/AA1) and VP2 (Met−/AA1−).

### Inverted Terminal Repeats (ITRs)

The AAV particles of the present disclosure include a viral genome with at least one ITR region and a payload region. In certain embodiments, the viral genome has two ITRs. These two ITRs flank the payload region at the 5′ and 3′ ends. The ITRs function as origins of replication including recognition sites for replication. ITRs include sequence regions which can be complementary and symmetrically arranged. ITRs incorporated into viral genomes of the present disclosure may be included of naturally occurring polynucleotide sequences or recombinantly derived polynucleotide sequences.

The ITRs may be derived from the same serotype as the capsid, or a derivative thereof. The ITR may be of a different serotype than the capsid. In certain embodiments, the AAV particle has more than one ITR. In a non-limiting example, the AAV particle has a viral genome including two ITRs. In certain embodiments, the ITRs are of the same serotype as one another. In another embodiment, the ITRs are of different serotypes. Non-limiting examples include zero, one or both of the ITRs having the same serotype as the capsid. In certain embodiments both ITRs of the viral genome of the AAV particle are AAV2 ITRs.

Independently, each ITR may be about 100 to about 150 nucleotides in length. An ITR may be about 100-105 nucleotides in length, 106-110 nucleotides in length, 111-115 nucleotides in length, 116-120 nucleotides in length, 121-125 nucleotides in length, 126-130 nucleotides in length, 131-135 nucleotides in length, 136-140 nucleotides in length, 141-145 nucleotides in length or 146-150 nucleotides in length. In certain embodiments, the ITRs are 140-142 nucleotides in length. Non-limiting examples of ITR length are 102, 130, 140, 141, 142, 145 nucleotides in length, and those having at least 95% identity thereto.

In certain embodiments, each ITR may be 141 nucleotides in length. In certain embodiments, each ITR may be 130 nucleotides in length. In certain embodiments, each ITR may be 119 nucleotides in length.

In certain embodiments, the AAV particles include two ITRs and one ITR is 141 nucleotides in length and the other ITR is 130 nucleotides in length. In certain embodiments, the AAV particles include two ITRs and both ITR are 141 nucleotides in length.

Independently, each ITR may be about 75 to about 175 nucleotides in length. The ITR may, independently, have a length such as, but not limited to, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, and 175 nucleotides. The length of the ITR for the viral genome may be 75-80, 75-85, 75-100, 80-85, 80-90, 80-105, 85-90, 85-95, 85-110, 90-95, 90-100, 90-115, 95-100, 95-105, 95-120, 100-105, 100-110, 100-125, 105-110, 105-115, 105-130, 110-115, 110-120, 110-135, 115-120, 115-125, 115-140, 120-125, 120-130, 120-145, 125-130, 125-135, 125-150, 130-135, 130-140, 130-155, 135-140, 135-145, 135-160, 140-145, 140-150, 140-165, 145-150, 145-155, 145-170, 150-155, 150-160, 150-175, 155-160, 155-165, 160-165, 160-170, 165-170, 165-175, and 170-175 nucleotides. As a non-limiting example, the viral genome comprises an ITR that is about 105 nucleotides in length. As a non-limiting example, the viral genome comprises an ITR that is about 141 nucleotides in length. As a non-limiting example, the viral genome comprises an ITR that is about 130 nucleotides in length. As a non-limiting example, the viral genome comprises an ITR that is about 105 nucleotides in length and 141 nucleotides in length. As a non-limiting example, the viral genome comprises an ITR that is about 105 nucleotides in length and 130 nucleotides in length. As a non-limiting example, the viral genome comprises an ITR that is about 130 nucleotides in length and 141 nucleotides in length.

### Genome Size

In certain embodiments, the AAV particle which includes a payload described herein may be single stranded or double stranded vector genome. The size of the vector genome may be small, medium, large or the maximum size. Additionally, the vector genome may include a promoter and a polyA tail.

In certain embodiments, the vector genome which includes a payload described herein may be a small single stranded vector genome. A small single stranded vector genome may be 2.1 to 3.5 kb in size such as about 2.1, 2.2, 2.3, 2.4, 2.5, 2.6, 2.7, 2.8, 2.9, 3.0, 3.1, 3.2, 3.3, 3.4, and 3.5 kb in size. As a non-limiting example, the small single stranded vector genome may be 3.2 kb in size. As another non-limiting example, the small single stranded vector genome may be 2.2 kb in size. Additionally, the vector genome may include a promoter and a polyA tail.

In certain embodiments, the vector genome which includes a payload described herein may be a small double stranded vector genome. A small double stranded vector genome may be 1.3 to 1.7 kb in size such as about 1.3, 1.4, 1.5, 1.6, and 1.7 kb in size. As a non-limiting example, the small double stranded vector genome may be 1.6 kb in size. Additionally, the vector genome may include a promoter and a polyA tail.

In certain embodiments, the vector genome which includes a payload described herein e.g., polynucleotide, siRNA, or dsRNA, may be a medium single stranded vector genome. A medium single stranded vector genome may be 3.6 to 4.3 kb in size such as about 3.6, 3.7, 3.8, 3.9, 4.0, 4.1, 4.2 and 4.3 kb in size. As a non-limiting example, the medium single stranded vector genome may be 4.0 kb in size. Additionally, the vector genome may include a promoter and a polyA tail.

In certain embodiments, the vector genome which includes a payload described herein may be a medium double stranded vector genome. A medium double stranded vector genome may be 1.8 to 2.1 kb in size such as about 1.8, 1.9, 2.0, and 2.1 kb in size. As a non-limiting example, the medium double stranded vector genome may be 2.0 kb in size. Additionally, the vector genome may include a promoter and a polyA tail.

In certain embodiments, the vector genome which includes a payload described herein may be a large single stranded vector genome. A large single stranded vector genome may be 4.4 to 6.0 kb in size such as about 4.4, 4.5, 4.6, 4.7, 4.8, 4.9, 5.0, 5.1, 5.2, 5.3, 5.4, 5.5, 5.6, 5.7, 5.8, 5.9 and 6.0 kb in size. As a non-limiting example, the large single stranded vector genome may be 4.7 kb in size. As another non-limiting example, the large single stranded vector genome may be 4.8 kb in size. As yet another non-limiting example, the large single stranded vector genome may be 6.0 kb in size. Additionally, the vector genome may include a promoter and a polyA tail.

In certain embodiments, the vector genome which includes a payload described herein may be a large double stranded vector genome. A large double stranded vector genome may be 2.2 to 3.0 kb in size such as about 2.2, 2.3, 2.4, 2.5, 2.6, 2.7, 2.8, 2.9 and 3.0 kb in size. As a non-limiting example, the large double stranded vector genome may be 2.4 kb in size. Additionally, the vector genome may include a promoter and a polyA tail.

### Vector Genome Regions: Filler Region

The AAV particles of the present disclosure include a viral genome with at least one filler region. The filler region(s) may, independently, have a length such as, but not limited to, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1604, 1605, 1606, 1607, 1608, 1609, 1610, 1611, 1612, 1613, 1614, 1615, 1616, 1617, 1618, 1619, 1620, 1621, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1649, 1650, 1651, 1652, 1653, 1654, 1655, 1656, 1657, 1658, 1659, 1660, 1661, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, 1749, 1750, 1751, 1752, 1753, 1754, 1755, 1756, 1757, 1758, 1759, 1760, 1761, 1762, 1763, 1764, 1765, 1766, 1767, 1768, 1769, 1770, 1771, 1772, 1773, 1774, 1775, 1776, 1777, 1778, 1779, 1780, 1781, 1782, 1783, 1784, 1785, 1786, 1787, 1788, 1789, 1790, 1791, 1792, 1793, 1794, 1795, 1796, 1797, 1798, 1799, 1800, 1801, 1802, 1803, 1804, 1805, 1806, 1807, 1808, 1809, 1810, 1811, 1812, 1813, 1814, 1815, 1816, 1817, 1818, 1819, 1820, 1821, 1822, 1823, 1824, 1825, 1826, 1827, 1828, 1829, 1830, 1831, 1832, 1833, 1834, 1835, 1836, 1837, 1838, 1839, 1840, 1841, 1842, 1843, 1844, 1845, 1846, 1847, 1848, 1849, 1850, 1851, 1852, 1853, 1854, 1855, 1856, 1857, 1858, 1859, 1860, 1861, 1862, 1863, 1864, 1865, 1866, 1867, 1868, 1869, 1870, 1871, 1872, 1873, 1874, 1875, 1876, 1877, 1878, 1879, 1880, 1881, 1882, 1883, 1884, 1885, 1886, 1887, 1888, 1889, 1890, 1891, 1892, 1893, 1894, 1895, 1896, 1897, 1898, 1899, 1900, 1901, 1902, 1903, 1904, 1905, 1906, 1907, 1908, 1909, 1910, 1911, 1912, 1913, 1914, 1915, 1916, 1917, 1918, 1919, 1920, 1921, 1922, 1923, 1924, 1925, 1926, 1927, 1928, 1929, 1930, 1931, 1932, 1933, 1934, 1935, 1936, 1937, 1938, 1939, 1940, 1941, 1942, 1943, 1944, 1945, 1946, 1947, 1948, 1949, 1950, 1951, 1952, 1953, 1954, 1955, 1956, 1957, 1958, 1959, 1960, 1961, 1962, 1963, 1964, 1965, 1966, 1967, 1968, 1969, 1970, 1971, 1972, 1973, 1974, 1975, 1976, 1977, 1978, 1979, 1980, 1981, 1982, 1983, 1984, 1985, 1986, 1987, 1988, 1989, 1990, 1991, 1992, 1993, 1994, 1995, 1996, 1997, 1998, 1999, 2000, 2001, 2002, 2003, 2004, 2005, 2006, 2007, 2008, 2009, 2010, 2011, 2012, 2013, 2014, 2015, 2016, 2017, 2018, 2019, 2020, 2021, 2022, 2023, 2024, 2025, 2026, 2027, 2028, 2029, 2030, 2031, 2032, 2033, 2034, 2035, 2036, 2037, 2038, 2039, 2040, 2041, 2042, 2043, 2044, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2097, 2098, 2099, 2100, 2101, 2102, 2103, 2104, 2105, 2106, 2107, 2108, 2109, 2110, 2111, 2112, 2113, 2114, 2115.2116, 2117, 2118, 2119, 2120, 2121, 2122, 2123, 2124, 2125, 2126, 2127, 2128, 2129, 2130, 2131, 2132, 2133, 2134, 2135, 2136, 2137, 2138, 2139, 2140, 2141, 2142, 2143, 2144, 2145, 2146, 2147, 2148, 2149, 2150, 2151, 2152, 2153, 2154, 2155, 2156, 2157, 2158, 2159, 2160, 2161, 2162, 2163, 2164, 2165, 2166, 2167, 2168, 2169, 2170, 2171, 2172, 2173, 2174, 2175, 2176, 2177, 2178, 2179, 2180, 2181, 2182, 2183, 2184, 2185, 2186, 2187, 2188, 2189, 2190, 2191, 2192, 2193, 2194, 2195, 2196, 2197, 2198, 2199, 2200, 2201, 2202, 2203, 2204, 2205, 2206, 2207, 2208, 2209, 2210, 2211, 2212, 2213, 2214, 2215, 2216, 2217, 2218, 2219, 2220, 2221, 2222, 2223, 2224, 2225, 2226, 2227, 2228, 2229, 2230, 2231, 2232, 2233, 2234, 2235, 2236, 2237, 2238, 2239, 2240, 2241, 2242, 2243, 2244, 2245, 2246, 2247, 2248, 2249, 2250, 2251, 2252, 2253, 2254, 2255, 2256, 2257, 2258, 2259, 2260, 2261, 2262, 2263, 2264, 2265, 2266, 2267, 2268, 2269, 2270, 2271, 2272, 2273, 2274, 2275, 2276, 2277, 2278, 2279, 2280, 2281, 2282, 2283, 2284, 2285, 2286, 2287, 2288, 2289, 2290, 2291, 2292, 2293, 2294, 2295, 2296, 2297, 2298, 2299, 2300, 2301, 2302, 2303, 2304, 2305, 2306, 2307, 2308, 2309, 2310, 2311, 2312, 2313, 2314, 2315, 2316, 2317, 2318, 2319, 2320, 2321, 2322, 2323, 2324, 2325, 2326, 2327, 2328, 2329, 2330, 2331, 2332, 2333, 2334, 2335, 2336, 2337, 2338, 2339, 2340, 2341, 2342, 2343, 2344, 2345, 2346, 2347, 2348, 2349, 2350, 2351, 2352, 2353, 2354, 2355, 2356, 2357, 2358, 2359, 2360, 2361, 2362, 2363, 2364, 2365, 2366, 2367, 2368, 2369, 2370, 2371, 2372, 2373, 2374, 2375, 2376, 2377, 2378, 2379, 2380, 2381, 2382, 2383, 2384, 2385, 2386, 2387, 2388, 2389, 2390, 2391, 2392, 2393, 2394, 2395, 2396, 2397, 2398, 2399, 2400, 2401, 2402, 2403, 2404, 2405, 2406, 2407, 2408, 2409, 2410, 2411, 2412, 2413, 2414, 2415, 2416, 2417, 2418, 2419, 2420, 2421, 2422, 2423, 2424, 2425, 2426, 2427, 2428, 2429, 2430, 2431, 2432, 2433, 2434, 2435, 2436, 2437, 2438, 2439, 2440, 2441, 2442, 2443, 2444, 2445, 2446, 2447, 2448, 2449, 2450, 2451, 2452, 2453, 2454, 2455, 2456, 2457, 2458, 2459, 2460, 2461, 2462, 2463, 2464, 2465, 2466, 2467, 2468, 2469, 2470, 2471, 2472, 2473, 2474, 2475, 2476, 2477, 2478, 2479, 2480, 2481, 2482, 2483, 2484, 2485, 2486, 2487, 2488, 2489, 2490, 2491, 2492, 2493, 2494, 2495, 2496, 2497, 2498, 2499, 2500, 2501, 2502, 2503, 2504, 2505, 2506, 2507, 2508, 2509, 2510, 2511, 2512, 2513, 2514, 2515, 2516, 2517, 2518, 2519, 2520, 2521, 2522, 2523, 2524, 2525, 2526, 2527, 2528, 2529, 2530, 2531, 2532, 2533, 2534, 2535, 2536, 2537, 2538, 2539, 2540, 2541, 2542, 2543, 2544, 2545, 2546, 2547, 2548, 2549, 2550, 2551, 2552, 2553, 2554, 2555, 2556, 2557, 2558, 2559, 2560, 2561, 2562, 2563, 2564, 2565, 2566, 2567, 2568, 2569, 2570, 2571, 2572, 2573, 2574, 2575, 2576, 2577, 2578, 2579, 2580, 2581, 2582, 2583, 2584, 2585, 2586, 2587, 2588, 2589, 2590, 2591, 2592, 2593, 2594, 2595, 2596, 2597, 2598, 2599, 2600, 2601, 2602, 2603, 2604, 2605, 2606, 2607, 2608, 2609, 2610, 2611, 2612, 2613, 2614, 2615, 2616, 2617, 2618, 2619, 2620, 2621, 2622, 2623, 2624, 2625, 2626, 2627, 2628, 2629, 2630, 2631, 2632, 2633, 2634, 2635, 2636, 2637, 2638, 2639, 2640, 2641, 2642, 2643, 2644, 2645, 2646, 2647, 2648, 2649, 2650, 2651, 2652, 2653, 2654, 2655, 2656, 2657, 2658, 2659, 2660, 2661, 2662, 2663, 2664, 2665, 2666, 2667, 2668, 2669, 2670, 2671, 2672, 2673, 2674, 2675, 2676, 2677, 2678, 2679, 2680, 2681, 2682, 2683, 2684, 2685, 2686, 2687, 2688, 2689, 2690, 2691, 2692, 2693, 2694, 2695, 2696, 2697, 2698, 2699, 2700, 2701, 2702, 2703, 2704, 2705, 2706, 2707, 2708, 2709, 2710, 2711, 2712, 2713, 2714, 2715, 2716, 2717, 2718, 2719, 2720, 2721, 2722, 2723, 2724, 2725, 2726, 2727, 2728, 2729, 2730, 2731, 2732, 2733, 2734, 2735, 2736, 2737, 2738, 2739, 2740, 2741, 2742, 2743, 2744, 2745, 2746, 2747, 2748, 2749, 2750, 2751, 2752, 2753, 2754, 2755, 2756, 2757, 2758, 2759, 2760, 2761, 2762, 2763, 2764, 2765, 2766, 2767, 2768, 2769, 2770, 2771, 2772, 2773, 2774, 2775, 2776, 2777, 2778, 2779, 2780, 2781, 2782, 2783, 2784, 2785, 2786, 2787, 2788, 2789, 2790, 2791, 2792, 2793, 2794, 2795, 2796, 2797, 2798, 2799, 2800, 2801, 2802, 2803, 2804, 2805, 2806, 2807, 2808, 2809, 2810, 2811, 2812, 2813, 2814, 2815, 2816, 2817, 2818, 2819, 2820, 2821, 2822, 2823, 2824, 2825, 2826, 2827, 2828, 2829, 2830, 2831, 2832, 2833, 2834, 2835, 2836, 2837, 2838, 2839, 2840, 2841, 2842, 2843, 2844, 2845, 2846, 2847, 2848, 2849, 2850, 2851, 2852, 2853, 2854, 2855, 2856, 2857, 2858, 2859, 2860, 2861, 2862, 2863, 2864, 2865, 2866, 2867, 2868, 2869, 2870, 2871, 2872, 2873, 2874, 2875, 2876, 2877, 2878, 2879, 2880, 2881, 2882, 2883, 2884, 2885, 2886, 2887, 2888, 2889, 2890, 2891, 2892, 2893, 2894, 2895, 2896, 2897, 2898, 2899, 2900, 2901, 2902, 2903, 2904, 2905, 2906, 2907, 2908, 2909, 2910, 2911, 2912, 2913, 2914, 2915, 2916, 2917, 2918, 2919, 2920, 2921, 2922, 2923, 2924, 2925, 2926, 2927, 2928, 2929, 2930, 2931, 2932, 2933, 2934, 2935, 2936, 2937, 2938, 2939, 2940, 2941, 2942, 2943, 2944, 2945, 2946, 2947, 2948, 2949, 2950, 2951, 2952, 2953, 2954, 2955, 2956, 2957, 2958, 2959, 2960, 2961, 2962, 2963, 2964, 2965, 2966, 2967, 2968, 2969, 2970, 2971, 2972, 2973, 2974, 2975, 2976, 2977, 2978, 2979, 2980, 2981, 2982, 2983, 2984, 2985, 2986, 2987, 2988, 2989, 2990, 2991, 2992, 2993, 2994, 2995, 2996, 2997, 2998, 2999, 3000, 3001, 3002, 3003, 3004, 3005, 3006, 3007, 3008, 3009, 3010, 3011, 3012, 3013, 3014, 3015, 3016, 3017, 3018, 3019, 3020, 3021, 3022, 3023, 3024, 3025, 3026, 3027, 3028, 3029, 3030, 3031, 3032, 3033, 3034, 3035, 3036, 3037, 3038, 3039, 3040, 3041, 3042, 3043, 3044, 3045, 3046, 3047, 3048, 3049, 3050, 3051, 3052, 3053, 3054, 3055, 3056, 3057, 3058, 3059, 3060, 3061, 3062, 3063, 3064, 3065, 3066, 3067, 3068, 3069, 3070, 3071, 3072, 3073, 3074, 3075, 3076, 3077, 3078, 3079, 3080, 3081, 3082, 3083, 3084, 3085, 3086, 3087, 3088, 3089, 3090, 3091, 3092, 3093, 3094, 3095, 3096, 3097, 3098, 3099, 3100, 3101, 3102, 3103, 3104, 3105, 3106, 3107, 3108, 3109, 3110, 3111, 3112, 3113, 3114, 3115, 3116, 3117, 3118, 3119, 3120, 3121, 3122, 3123, 3124, 3125, 3126, 3127, 3128, 3129, 3130, 3131, 3132, 3133, 3134, 3135, 3136, 3137, 3138, 3139, 3140, 3141, 3142, 3143, 3144, 3145, 3146, 3147, 3148, 3149, 3150, 3151, 3152, 3153, 3154, 3155, 3156, 3157, 3158, 3159, 3160, 3161, 3162, 3163, 3164, 3165, 3166, 3167, 3168, 3169, 3170, 3171, 3172, 3173, 3174, 3175, 3176, 3177, 3178, 3179, 3180, 3181, 3182, 3183, 3184, 3185, 3186, 3187, 3188, 3189, 3190, 3191, 3192, 3193, 3194, 3195, 3196, 3197, 3198, 3199, 3200, 3201, 3202, 3203, 3204, 3205, 3206, 3207, 3208, 3209, 3210, 3211, 3212, 3213, 3214, 3215, 3216, 3217, 3218, 3219, 3220, 3221, 3222, 3223, 3224, 3225, 3226, 3227, 3228, 3229, 3230, 3231, 3232, 3233, 3234, 3235, 3236, 3237, 3238, 3239, 3240, 3241, 3242, 3243, 3244, 3245, 3246, 3247, 3248, 3249, and 3250 nucleotides. The length of any filler region for the viral genome may be 50-100, 100-150, 150-200, 200-250, 250-300, 300-350, 350-400, 400-450, 450-500, 500-550, 550-600, 600-650, 650-700, 700-750, 750-800, 800-850, 850-900, 900-950, 950-1000, 1000-1050, 1050-1100, 1100-1150, 1150-1200, 1200-1250, 1250-1300, 1300-1350, 1350-1400, 1400-1450, 1450-1500, 1500-1550, 1550-1600, 1600-1650, 1650-1700, 1700-1750, 1750-1800, 1800-1850, 1850-1900, 1900-1950, 1950-2000, 2000-2050, 2050-2100, 2100-2150, 2150-2200, 2200-2250, 2250-2300, 2300-2350, 2350-2400, 2400-2450, 2450-2500, 2500-2550, 2550-2600, 2600-2650, 2650-2700, 2700-2750, 2750-2800, 2800-2850, 2850-2900, 2900-2950, 2950-3000, 3000-3050, 3050-3100, 3100-3150, 3150-3200, and 3200-3250 nucleotides. As a non-limiting example, the viral genome comprises a filler region that is about 55 nucleotides in length. As a non-limiting example, the viral genome comprises a filler region that is about 56 nucleotides in length. As a non-limiting example, the viral genome comprises a filler region that is about 97 nucleotides in length. As a non-limiting example, the viral genome comprises a filler region that is about 103 nucleotides in length. As a non-limiting example, the viral genome comprises a filler region that is about 105 nucleotides in length. As a non-limiting example, the viral genome comprises a filler region that is about 357 nucleotides in length. As a non-limiting example, the viral genome comprises a filler region that is about 363 nucleotides in length. As a non-limiting example, the viral genome comprises a filler region that is about 712 nucleotides in length. As a non-limiting example, the viral genome comprises a filler region that is about 714 nucleotides in length. As a non-limiting example, the viral genome comprises a filler region that is about 1203 nucleotides in length. As a non-limiting example, the viral genome comprises a filler region that is about 1209 nucleotides in length. As a non-limiting example, the viral genome comprises a filler region that is about 1512 nucleotides in length. As a non-limiting example, the viral genome comprises a filler region that is about 1519 nucleotides in length. As a non-limiting example, the viral genome comprises a filler region that is about 2395 nucleotides in length. As a non-limiting example, the viral genome comprises a filler region that is about 2403 nucleotides in length. As a non-limiting example, the viral genome comprises a filler region that is about 2405 nucleotides in length. As a non-limiting example, the viral genome comprises a filler region that is about 3013 nucleotides in length. As a non-limiting example, the viral genome comprises a filler region that is about 3021 nucleotides in length.

In one embodiment, the filler region is 714 nucleotides in length.

### Vector Genome Regions: Multiple Cloning Site (MCS) Region

The AAV particles of the present disclosure include a viral genome with at least one multiple cloning site (MCS) region. The MCS region(s) may, independently, have a length such as, but not limited to, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, and 150 nucleotides. The length of the MCS region for the viral genome may be 2-10, 5-10, 5-15, 10-20, 10-30, 10-40, 15-20, 15-25, 20-30, 20-40, 20-50, 25-30, 25-35, 3040, 30-50, 30-60, 35-40, 3545, 40-50, 40-60, 40-70, 45-50, 45-55, 50-60, 50-70, 50-80, 55-60, 55-65, 60-70, 60-80, 60-90, 65-70, 65-75, 70-80, 70-90, 70-100, 75-80, 75-85, 80-90, 80-100, 80-110, 85-90, 85-95, 90-100, 90-110, 90-120, 95-100, 95-105, 100-110, 100-120, 100-130, 105-110, 105-115, 110-120, 110-130, 110-140, 115-120, 115-125, 120-130, 120-140, 120-150, 125-130, 125-135, 130-140, 130-150, 135-140, 135-145, 140-150, and 145-150 nucleotides. As a non-limiting example, the viral genome comprises a MCS region that is about 5 nucleotides in length. As a non-limiting example, the viral genome comprises a MCS region that is about 10 nucleotides in length. As a non-limiting example, the viral genome comprises a MCS region that is about 14 nucleotides in length. As a non-limiting example, the viral genome comprises a MCS region that is about 18 nucleotides in length. As a non-limiting example, the viral genome comprises a MCS region that is about 73 nucleotides in length. As a non-limiting example, the viral genome comprises a MCS region that is about 121 nucleotides in length.

In one embodiment, the MCS region is 5 nucleotides in length.

In one embodiment, the MCS region is 10 nucleotides in length.

### Vector Genome Regions: Promoter and Enhancer Regions

The AAV particles of the present disclosure include a viral genome with at least one promoter region. The promoter region(s) may, independently, have a length such as, but not limited to, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, and 600 nucleotides. The length of the promoter region for the viral genome may be 4-10, 10-20, 10-50, 20-30, 30-40, 40-50, 50-60, 50-100, 60-70, 70-80, 80-90, 90-100, 100-110, 100-150, 110-120, 120-130, 130-140, 140-150, 150-160, 150-200, 160-170, 170-180, 180-190, 190-200, 200-210, 200-250, 210-220, 220-230, 230-240, 240-250, 250-260, 250-300, 260-270, 270-280, 280-290, 290-300, 300-310, 300-350, 310-320, 320-330, 330-340, 340-350, 350-360, 350-400, 360-370, 370-380, 380-390, 390-400, 400-410, 400-450, 410-420, 420-430, 430-440, 440-450, 450-460, 450-500, 460-470, 470-480, 480-490, 490-500, 500-510, 500-550, 510-520, 520-530, 530-540, 540-550, 550-560, 550-600, 560-570, 570-580, 580-590, and 590-600 nucleotides. As a non-limiting example, the viral genome comprises a promoter region that is about 4 nucleotides in length. As a non-limiting example, the viral genome comprises a promoter region that is about 17 nucleotides in length. As a non-limiting example, the viral genome comprises a promoter region that is about 204 nucleotides in length. As a non-limiting example, the viral genome comprises a promoter region that is about 219 nucleotides in length. As a non-limiting example, the viral genome comprises a promoter region that is about 260 nucleotides in length. As a non-limiting example, the viral genome comprises a promoter region that is about 303 nucleotides in length. As a non-limiting example, the viral genome comprises a promoter region that is about 382 nucleotides in length. As a non-limiting example, the viral genome comprises a promoter region that is about 588 nucleotides in length.

In one embodiment, the promoter region is derived from a CBA promoter sequence. As a non-limiting example, the promoter is 260 nucleotides in length.

The AAV particles of the present disclosure include a viral genome with at least one enhancer region. The enhancer region(s) may, independently, have a length such as, but not limited to, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, and 400 nucleotides. The length of the enhancer region for the viral genome may be 300-310, 300-325, 305-315, 310-320, 315-325, 320-330, 325-335, 325-350, 330-340, 335-345, 340-350, 345-355, 350-360, 350-375, 355-365, 360-370, 365-375, 370-380, 375-385, 375-400, 380-390, 385-395, and 390-400 nucleotides. As a non-limiting example, the viral genome comprises an enhancer region that is about 303 nucleotides in length. As a non-limiting example, the viral genome comprises an enhancer region that is about 382 nucleotides in length.

In one embodiment, the enhancer region is derived from a CMV enhancer sequence. As a non-limiting example, the CMV enhancer is 382 nucleotides in length.

### Vector Genome Region: Exon and Intron Regions

The AAV particles of the present disclosure include a viral genome with at least one exon region. The exon region(s) may, independently, have a length such as, but not limited to, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, and 150 nucleotides. The length of the exon region for the viral genome may be 2-10, 5-10, 5-15, 10-20, 10-30, 10-40, 15-20, 15-25, 20-30, 2040, 20-50, 25-30, 25-35, 30-40, 30-50, 30-60, 35-40, 35-45, 40-50, 40-60, 40-70, 45-50, 45-55, 50-60, 50-70, 50-80, 55-60, 55-65, 60-70, 60-80, 60-90, 65-70, 65-75, 70-80, 70-90, 70-100, 75-80, 75-85, 80-90, 80-100, 80-110, 85-90, 85-95, 90-100, 90-110, 90-120, 95-100, 95-105, 100-110, 100-120, 100-130, 105-110, 105-115, 110-120, 110-130, 110-140, 115-120, 115-125, 120-130, 120-140, 120-150, 125-130, 125-135, 130-140, 130-150, 135-140, 135-145, 140-150, and 145-150 nucleotides. As a non-limiting example, the viral genome comprises an exon region that is about 53 nucleotides in length. As a non-limiting example, the viral genome comprises an exon region that is about 134 nucleotides in length.

The AAV particles of the present disclosure include a viral genome with at least one intron region. The intron region(s) may, independently, have a length such as, but not limited to, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213.214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, and 350 nucleotides. The length of the intron region for the viral genome may be 25-35, 25-50, 3545, 45-55, 50-75, 55-65, 65-75, 75-85, 75-100, 85-95, 95-105, 100-125, 105-115, 115-125, 125-135, 125-150, 135-145, 145-155, 150-175, 155-165, 165-175, 175-185, 175-200, 185-195, 195-205, 200-225, 205-215, 215-225, 225-235, 225-250, 235-245, 245-255, 250-275, 255-265, 265-275, 275-285, 275-300, 285-295, 295-305, 300-325, 305-315, 315-325, 325-335, 325-350, and 335-345 nucleotides. As a non-limiting example, the viral genome comprises an intron region that is about 32 nucleotides in length. As a non-limiting example, the viral genome comprises an intron region that is about 172 nucleotides in length. As a non-limiting example, the viral genome comprises an intron region that is about 201 nucleotides in length. As a non-limiting example, the viral genome comprises an intron region that is about 347 nucleotides in length.

In one embodiment, the intron region is derived from a SV40 intron sequence. As a non-limiting example, the intron is 172 nucleotides in length.

### II. AAV Production

### General Production Process and Components

Viral production cells for the production of rAAV particles generally include mammalian cell types. However, mammalian cells present several complications to the large-scale production of rAAV particles, including general low yield of viral-particles-per-replication-cell as well as high risks for undesirable contamination from other mammalian biomaterials in the viral production cell. As a result, insect cells have become an alternative vehicle for large-scale production of rAAV particles.

AAV production systems using insect cells also present a range of complications. For example, high-yield production of rAAV particles often requires a lower expression of Rep78 compared to Rep52. Controlling the relative expression of Rep78 and Rep52 in insect cells thus requires carefully designed control mechanisms within the Rep operon. These control mechanisms can include individually optimized insect cell promoters, such as ΔIE1 promoters for Rep78 and PolH promoters for Rep52, or the division of the Rep-encoding nucleotide sequences onto independently optimized sequences or constructs. However, implementation of these control mechanisms often leads to reduced rAAV particle yield or to structurally unstable virions.

In another example, production of rAAV particles requires VP1, VP2 and VP3 proteins which assemble to form the AAV capsid. High-yield production of rAAV particles requires optimized ratios of VP1, VP2 and VP3, which should generally be around 1:1:10, respectively, but can vary from 1-2 for VP1 and/or 1-2 for VP2, relative to 10 VP3 copies. This ratio is important for the quality of the capsid, as too much VP1 destabilizes the capsid and too little VP1 will decrease the infectivity of the virus.

Wild type AAV use a deficient splicing method to control VP1 expression; a weak start codon (ACG) with special surrounding (“Kozak” sequence) to control VP2; and a standard start codon (ATG) for VP3 expression. However, in some baculovirus systems, the mammalian splicing sequences are not always recognized and unable to properly control the production of VP1, VP2 and VP3. Consequently, neighboring nucleotides and the ACG start sequence from VP2 can be used to drive capsid protein production. Unfortunately, for most of the AAV serotypes, this method creates a capsid with a lower ratio of VP1 compared to VP2 (<1 relative to 10 VP3 copies). To more effectively control the production of VP proteins, non-canonical or start codons have been used, like TTG, GTG or CTG. However, these start codons are considered suboptimal by those in the art relative to the wild type ATG or ACG start codons (See, WO2007046703 and WO2007148971, the contents of which are incorporated herein by reference in their entirety).

In another example, production of rAAV particles using a baculovirus/Sf9 system generally requires the widely used bacmid-based Baculovirus Expression Vector System (BEVs), which are not optimized for large-scale AAV production. Aberrant proteolytic degradation of viral proteins in the bacmid-based BEVs is an unexpected issue, precluding the reliable large-scale production of AAV capsid proteins using the baculovirus/Sf9 system.

There is continued need for methods and systems which allow for effective and efficient large scale (commercial) production of rAAV particles in mammalian and insect cells.

The details of one or more embodiments of the present disclosure are set forth in the accompanying description below. Other features, objects, and advantages of the present disclosure will be apparent from the description, drawings, and the claims. In the description, the singular forms also include the plural unless the context clearly dictates otherwise. Unless defined otherwise, all technical and scientific terms used herein have the same meaning as commonly understood by one of ordinary skill in the art to which this present disclosure belongs. In the case of conflict with disclosures incorporated by reference, the present express description will control.

In certain embodiments, the constructs, polynucleotides, polypeptides, vectors, serotypes, capsids formulations, or particles of the present disclosure may be, may include, may be modified by, may be used by, may be used for, may be used with, or may be produced with any sequence, element, construct, system, target or process described in one of the following International Publications: WO2016073693, WO2017023724, WO2018232055, WO2016077687, WO2016077689, WO2018204786, WO2017201258, WO2017201248, WO2018204803, WO2018204797, WO2017189959. WO2017189963. WO2017189964, WO2015191508, WO2016094783, WO20160137949, WO2017075335; the contents of which are each herein incorporated by reference in their entirety.

AAV production of the present disclosure includes processes and methods for producing AAV particles and viral vectors which can contact a target cell to deliver a payload, e.g., a recombinant viral construct, which includes a nucleotide encoding a payload molecule. In certain embodiments, the viral vectors are adeno-associated viral (AAV) vectors such as recombinant adeno-associated viral (rAAV) vectors. In certain embodiments, the AAV particles are adeno-associated viral (AAV) particles such as recombinant adeno-associated viral (rAAV) particles.

In certain embodiments, a process of the present disclosure includes production of viral particles in a viral production cell using a viral production system which includes at least one viral expression construct and at least one payload construct. The at least one viral expression construct and at least one payload construct can be co-transfected (e.g. dual transfection, triple transfection) into a viral production cell. The transfection is completed using standard molecular biology techniques known and routinely performed by a person skilled in the art. The viral production cell provides the cellular machinery necessary for expression of the proteins and other biomaterials necessary for producing the AAV particles, including Rep proteins which replicate the payload construct and Cap proteins which assemble to form a capsid that encloses the replicated payload constructs. The resulting AAV particle is extracted from the viral production cells and processed into a pharmaceutical preparation for administration.

Once administered, the AAV particles contacts a target cell and enters the cell in an endosome. The AAV particle releases from the endosome and subsequently contacts the nucleus of the target cell to deliver the payload construct. The payload construct, e.g., recombinant viral construct, is delivered to the nucleus of the target cell wherein the payload molecule encoded by the payload construct may be expressed.

In certain embodiments, the process for production of viral particles utilizes seed cultures of viral production cells that include one or more baculoviruses (e.g., a Baculoviral Expression Vector (BEV) or a baculovirus infected insect cell (BIIC) that has been transfected with a viral expression construct and a payload construct vector). In certain embodiments, the seed cultures are harvested, divided into aliquots and frozen, and may be used at a later time point to initiate an infection of a naïve population of production cells.

Large scale production of AAV particles may utilize a bioreactor. The use of a bioreactor allows for the precise measurement and/or control of variables that support the growth and activity of viral production cells such as mass, temperature, mixing conditions (impellor RPM or wave oscillation), CO₂ concentration, O₂ concentration, gas sparge rates and volumes, gas overlay rates and volumes, pH, Viable Cell Density (VCD), cell viability, cell diameter, and/or optical density (OD). In certain embodiments, the bioreactor is used for batch production in which the entire culture is harvested at an experimentally determined time point and AAV particles are purified. In another embodiment, the bioreactor is used for continuous production in which a portion of the culture is harvested at an experimentally determined time point for purification of AAV particles, and the remaining culture in the bioreactor is refreshed with additional growth media components.

AAV viral particles can be extracted from viral production cells in a process which includes cell lysis, clarification, sterilization, and purification. Cell lysis includes any process that disrupts the structure of the viral production cell, thereby releasing AAV particles. In certain embodiments cell lysis may include thermal shock, chemical, or mechanical lysis methods. Clarification can include the gross purification of the mixture of lysed cells, media components, and AAV particles. In certain embodiments, clarification includes centrifugation and/or filtration, including but not limited to depth end, tangential flow, and/or hollow fiber filtration.

The end result of viral production is a purified collection of AAV particles which include two components: (1) a payload construct (e.g., a recombinant viral construct) and (2) a viral capsid.

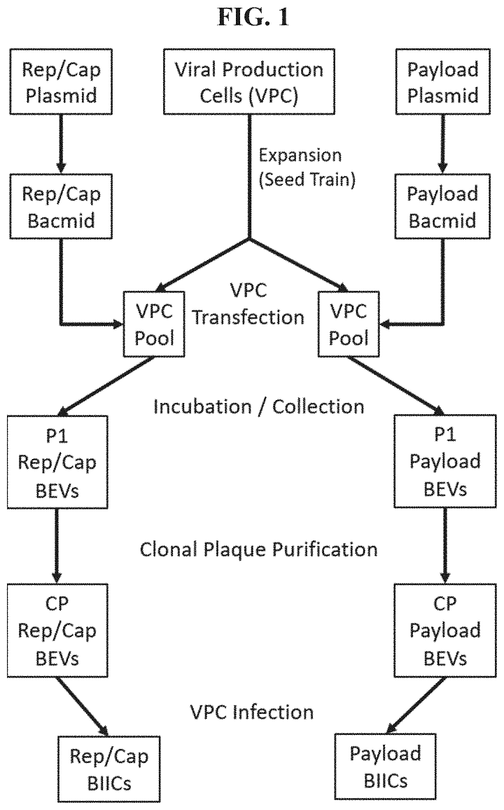

FIG. 1 shows a schematic for one embodiment of a system, and a flow diagram for one embodiment of a process, for producing baculovirus infected insect cells (BIICs) using Viral Production Cells (VPC) and plasmid constructs. Viral Production Cells (VPCs) from a Cell Bank (CB) are thawed and expanded to provide a target working volume and VPC concentration. The resulting pool of VPCs is split into a Rep/Cap VPC pool and a Payload VPC pool. One or more Rep/Cap plasmid constructs (viral expression constructs) are processed into Rep/Cap Bacmid polynucleotides and transfected into the Rep/Cap VPC pool. One or more Payload plasmid constructs (payload constructs) are processed into Payload Bacmid polynucleotides and transfected into the Payload VPC pool. The two VPC pools are incubated to produce P1 Rep/Cap Baculoviral Expression Vectors (BEVs) and P1 Payload BEVs. The two BEV pools are expanded into a collection of Plaques, with a single Plaque being selected for Clonal Plaque (CP) Purification (also referred to as Single Plaque Expansion). The process can include a single CP Purification step or can include multiple CP Purification steps either in series or separated by other processing steps. The one-or-more CP Purification steps provide a CP Rep/Cap BEV pool and a CP Payload BEV pool. These two BEV pools can then be stored and used for future production steps, or they can be then transfected into VPCs to produce a Rep/Cap BIIC pool and a Payload BIIC pool.

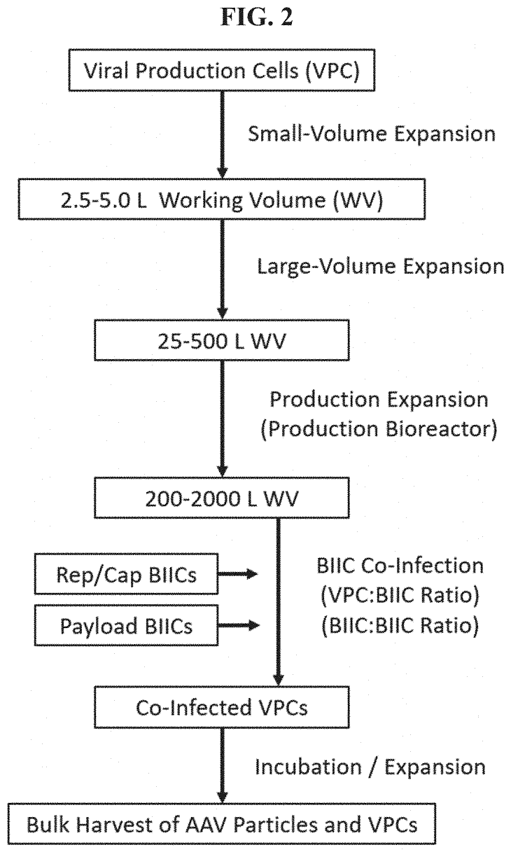

FIG. 2 shows one embodiment of a schematic for a system, and a flow diagram for one embodiment of a process, for producing AAV particles using Viral Production Cells (VPC) and baculovirus infected insect cells (BIICs). Viral Production Cells (VPCs) from a Cell Bank (CB) are thawed and expanded to provide a target working volume and VPC concentration. This expansion includes one or more small-volume expansion steps up to a working volume of 2500-5000 mL, followed by one or more large-volume expansion steps in large-scale bioreactors (e.g., Wave and/or N-1 bioreactors) up to a working volume of 25-500 L. The working volume of Viral Production Cells is seeded into a Production Bioreactor and can be further expanded to a working volume of 200-2000 L with a target VPC concentration for BIIC infection.

The working volume of VPCs in the Production Bioreactor is then co-infected with Rep/Cap BIICs and Payload BIICs, with a target VPC:BIIC ratio and a target BIIC:BIIC ratio. VCD infection can also utilize BEVs. The co-infected VPCs are incubated and expanded in the Production Bioreactor to produce a bulk harvest of AAV particles and VPCs.

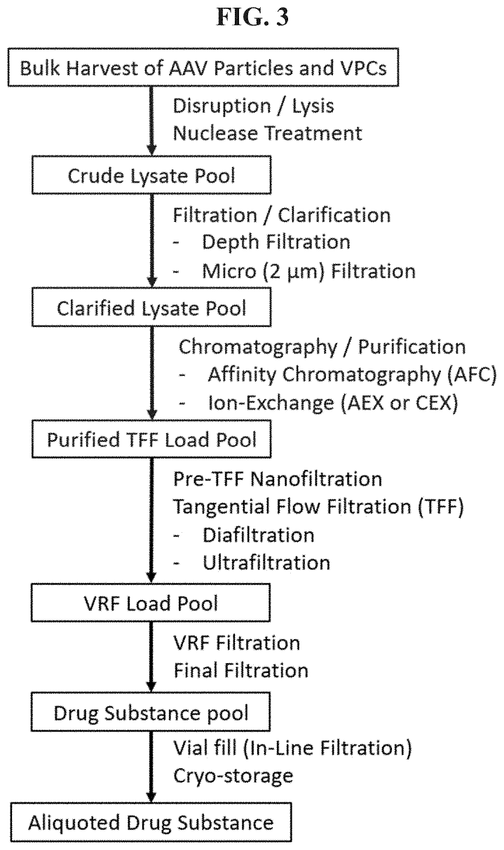

FIG. 3 shows schematic for one embodiment of a system, and a flow diagram for one embodiment of a process, for producing a Drug Substance by processing, clarifying, and purifying a bulk harvest of AAV particles and Viral Production Cells. A bulk harvest of AAV particles and VPCs (within a Production Bioreactor) are processed through cellular disruption and lysis (e.g., chemical lysis and/or mechanical lysis), followed by nuclease treatment of the lysis pool, thereby producing a crude lysate pool. The crude lysate pool is processed through one or more filtration and clarification steps, including depth filtration and microfiltration to provide a clarified lysate pool. The clarified lysate pool is processed through one or more chromatography and purification steps, including affinity chromatography (AFC) and ion-exchange chromatography (AEX or CEX) to provide a purified product pool. The purified product pool is then optionally processed through nanofiltration, and then through tangential flow filtration (TFF). The TFF process includes one or more diafiltration (DF) steps and one or more ultrafiltration (UF) steps, either in series or alternating. The product pool is further processed through viral retention filtration (VRF) and a final filtration step to provide a drug substance pool. The drug substance pool can be further filtered, then aliquoted into vials for storage and treatment.

### Viral Constructs

### Viral Expression Construct

The viral production system of the present disclosure includes one or more viral expression constructs which can be transfected/transduced into a viral production cell. A viral expression construct can contain parvoviral genes under control of one or more promoters. Parvoviral genes can include nucleotide sequences encoding non-structural AAV replication proteins, such as Rep genes which encode Rep52, Rep40, Rep68 or Rep78 proteins. Parvoviral genes can include nucleotide sequences encoding structural AAV proteins, such as Cap genes which encode VP1, VP2 and VP3 proteins.

In certain embodiments, a viral expression construct can include a Rep52-coding region; a Rep52-coding region is a nucleotide sequence which includes a Rep52 nucleotide sequence encoding a Rep52 protein. In certain embodiments, a viral expression construct can include a Rep78-coding region; a Rep78-coding region is a nucleotide sequence which includes a Rep78 nucleotide sequence encoding a Rep78 protein. In certain embodiments, a viral expression construct can include a Rep40-coding region; a Rep40-coding region is a nucleotide sequence which includes a Rep40 nucleotide sequence encoding a Rep40 protein. In certain embodiments, a viral expression construct can include a Rep68-coding region; a Rep68-coding region is a nucleotide sequence which includes a Rep68 nucleotide sequence encoding a Rep68 protein.

In certain embodiments, a viral expression construct can include a VP-coding region; a VP-coding region is a nucleotide sequence which includes a VP nucleotide sequence encoding VP1, VP2, VP3, or a combination thereof. In certain embodiments, a viral expression construct can include a VP1-coding region; a VP1-coding region is a nucleotide sequence which includes a VP1 nucleotide sequence encoding a VP1 protein. In certain embodiments, a viral expression construct can include a VP2-coding region; a VP2-coding region is a nucleotide sequence which includes a VP2 nucleotide sequence encoding a VP2 protein. In certain embodiments, a viral expression construct can include a VP3-coding region; a VP3-coding region is a nucleotide sequence which includes a VP3 nucleotide sequence encoding a VP3 protein.

Promoters can include, but are not limited to, baculovirus major late promoters, insect virus promoters, non-insect virus promoters, vertebrate virus promoters, nuclear gene promoters, chimeric promoters from one or more species including virus and non-virus elements, and/or synthetic promoters. In certain embodiments, a promoter can be selected from: Op-EI, EI, ΔEI, EI-1, pH, PIO, polh (polyhedron), ΔpolH, Dmhsp70, Hr1, Hsp70, 4xHsp27 EcRE+minimal Hsp70, IE, IE-1, ΔIE-1, ΔIE, p10, Δp10 (modified variations or derivatives of p10), p5, p19, p35, p40, and variations or derivatives thereof. In certain embodiments, a promoter can be selected from tissue-specific promoters, cell-type-specific promoters, cell-cycle-specific promoters, and variations or derivatives thereof. In certain embodiments, a promoter can be selected from: CMV promoter, an alpha 1-antitrypsin (α1-AT) promoter, a thyroid hormone-binding globulin promoter, a thyroxine-binding globlin (LPS) promoter, an HCR-ApoCII hybrid promoter, an HCR-hAAT hybrid promoter, an albumin promoter, an apolipoprotein E promoter, an α1-AT+EaIb promoter, a tumor-selective E2F promoter, a mononuclear blood IL-2 promoter, and variations or derivatives thereof. In certain embodiments, the promoter is a low-expression promoter sequence. In certain embodiments, the promoter is an enhanced-expression promoter sequence. In certain embodiments, the promoter can include Rep or Cap promoters as described in US Patent Application 20110136227, the contents of which are herein incorporated by reference in its entirety

In certain embodiments, a viral expression construct can include the same promoter in all nucleotide sequences. In certain embodiments, a viral expression construct can include the same promoter in two or more nucleotide sequences. In certain embodiments, a viral expression construct can include a different promoter in two or more nucleotide sequences. In certain embodiments, a viral expression construct can include a different promoter in all nucleotide sequences.

The viral production system of the present disclosure is not limited by the viral expression vector used to introduce the parvoviral functions into the virus replication cell. The presence of the viral expression construct in the virus replication cell need not be permanent. The viral expression constructs can be introduced by any means known, for example by chemical treatment of the cells, electroporation, or infection.

Viral expression constructs of the present disclosure may include any compound or formulation, biological or chemical, which facilitates transformation, transfection, or transduction of a cell with a nucleic acid. Exemplary biological viral expression constructs include plasmids, linear nucleic acid molecules, and recombinant viruses including baculovirus. Exemplary chemical vectors include lipid complexes. Viral expression constructs are used to incorporate nucleic acid sequences into virus replication cells in accordance with the present disclosure. (O'Reilly, David R., Lois K. Miller, and Verne A. Luckow. Baculovirus expression vectors: a laboratory manual. Oxford University Press, 1994); Maniatis et al., eds. Molecular Cloning. CSH Laboratory, NY, N.Y. (1982); and, Philiport and Scluber, eds. Liposoes as tools in Basic Research and Industry. CRC Press, Ann Arbor, Mich. (1995), the contents of each of which are herein incorporated by reference in its entirety.

In certain embodiments, the viral expression construct is an AAV expression construct which includes one or more nucleotide sequences encoding non-structural AAV replication proteins, structural AAV replication proteins, or a combination thereof.

In certain embodiments, the viral expression construct of the present disclosure may be a plasmid vector. In certain embodiments, the viral expression construct of the present disclosure may be a baculoviral construct.

The present disclosure is not limited by the number of viral expression constructs employed to produce AAV particles or viral vectors. In certain embodiments, one, two, three, four, five, six, or more viral expression constructs can be employed to produce AAV particles in viral production cells in accordance with the present disclosure. In one non-limiting example, five expression constructs may individually encode AAV VP1, AAV VP2, AAV VP3, Rep52, Rep78, and with an accompanying payload construct comprising a payload polynucleotide and at least one AAV ITR. In another embodiment, expression constructs may be employed to express, for example, Rep52 and Rep40, or Rep78 and Rep 68. Expression constructs may include any combination of VP1, VP2, VP3, Rep52/Rep40, and Rep78/Rep68 coding sequences.

In certain embodiments the viral expression construct encodes elements to optimize expression in certain cell types. In a further embodiment, the expression construct may include polh and/or ΔIE-1 insect transcriptional promoters, CMV mammalian transcriptional promoter, and/or p10 insect specific promoters for expression of a desired gene in a mammalian or insect cell.

In certain embodiments of the present disclosure, a viral expression construct may be used for the production of an AAV particles in insect cells. In certain embodiments, modifications may be made to the wild type AAV sequences of the capsid and/or rep genes, for example to improve attributes of the viral particle, such as increased infectivity or specificity, or to enhance production yields.

In certain embodiments, the viral expression construct may contain a nucleotide sequence which includes start codon region, such as a sequence encoding AAV capsid proteins which include one or more start codon regions. The start codon can be ATG or a non-ATG codon (i.e., a suboptimal start codon where the start codon of the AAV VP1 capsid protein is a non-ATG). In certain embodiments, the viral expression construct may contain a nucleotide sequence encoding the AAV capsid proteins where the start codon of the AAV VP1 capsid protein is a non-ATG, i.e., a suboptimal start codon, allowing the expression of a modified ratio of the viral capsid proteins in the insect cell production system, to provide improved infectivity of the host cell. In a non-limiting example, a viral expression construct of the present disclosure may contain a nucleic acid construct comprising a nucleotide sequence encoding AAV VP1, VP2, and VP3 capsid proteins, wherein the start codon for translation of the AAV VP1 capsid protein is CTG, TTG, or GTG, as described in U.S. Pat. No. 8,163,543, the contents of which are herein incorporated by reference in its entirety.

In certain embodiments, the viral expression construct can include an expression control region which includes an expression control sequence. In certain embodiments, the viral expression construct can include an IRES sequence region which includes an IRES nucleotide sequence encoding an internal ribosome entry sight (IRES). The internal ribosome entry sight (IRES) can be selected from the group consisting or: FMDV-IRES from Foot-and-Mouth-Disease virus, EMCV-IRES from Encephalomyocarditis virus, and combinations thereof.

In certain embodiments, the viral expression construct can include a 2A sequence region which comprises a 2A nucleotide sequence encoding a viral 2A peptide. A viral 2A sequence is a relatively short (approximately 20 amino acids) sequence which contains a consensus sequence of: Asp-Val/Ile-Glu-X-Asn-Pro-Gly-Pro. The sequence allows for co-translation of multiple polypeptides within a single open reading frame (ORF). As the ORF is translated, glycine and proline residues with the 2A sequence prevent the formation of a normal peptide bond, which results in ribosomal “skipping” and “self-cleavage” within the polypeptide chain. The viral 2A peptide can be selected from the group consisting of: F2A from Foot-and-Mouth-Disease virus, T2A from Thosea asigna virus, E2A from Equine rhinitis A virus, P2A from porcine teschovirus-1, BmCPV2A from cytoplasmic polyhedrosis virus, BmIFV 2A from B. mori flacherie virus, and combinations thereof.

In certain embodiments, the viral expression construct used for AAV production may contain a nucleotide sequence encoding the AAV capsid proteins where the initiation codon of the AAV VP1 capsid protein is a non-ATG, i.e., a suboptimal initiation codon, allowing the expression of a modified ratio of the viral capsid proteins in the production system, to provide improved infectivity of the host cell. In a non-limiting example, a viral construct vector may contain a nucleic acid construct comprising a nucleotide sequence encoding AAV VP1, VP2, and VP3 capsid proteins, wherein the initiation codon for translation of the AAV VP1 capsid protein is CTG, TTG, or GTG, as described in U.S. Pat. No. 8,163,543, the contents of which are herein incorporated by reference in its entirety.

In certain embodiments, the viral expression construct of the present disclosure may be a plasmid vector or a baculoviral construct that encodes the parvoviral rep proteins for expression in insect cells. In certain embodiments, a single coding sequence is used for the Rep78 and Rep52 proteins, wherein start codon for translation of the Rep78 protein is a suboptimal start codon, selected from the group consisting of ACG, TTG, CTG and GTG, that effects partial exon skipping upon expression in insect cells, as described in U.S. Pat. No. 8,512,981, the contents of which are herein incorporated by reference in their entirety, for example to promote less abundant expression of Rep78 as compared to Rep52, which may in that it promotes high vector yields.

In certain embodiments, the viral expression construct may be a plasmid vector or a baculoviral construct for the expression in insect cells that contains repeating codons with differential codon biases, for example to achieve improved ratios of Rep proteins, eg. Rep78 and Rep52 thereby improving large scale (commercial) production of viral expression construct and/or payload construct vectors in insect cells, as taught in U.S. Pat. No. 8,697,417, the contents of which are herein incorporated by reference in their entirety.

In another embodiment, improved ratios of rep proteins may be achieved using the method and constructs described in U.S. Pat. No. 8,642,314, the contents of which are herein incorporated by reference in their entirety.

In certain embodiments, the viral expression construct may encode mutant parvoviral Rep polypeptides which have one or more improved properties as compared with their corresponding wild type Rep polypeptide, such as the preparation of higher virus titers for large scale production. Alternatively, they may be able to allow the production of better-quality viral particles or sustain more stable production of virus. In a non-limiting example, the viral expression construct may encode mutant Rep polypeptides with a mutated nuclear localization sequence or zinc finger domain, as described in Patent Application US 20130023034, the contents of which are herein incorporated by reference in their entirety.

In certain embodiments, the viral expression construct may encode the components of a Parvoviral capsid with incorporated Gly-Ala repeat region, which may function as an immune invasion sequence, as described in US Patent Application 20110171262, the contents of which are herein incorporated by reference in its entirety.

In certain embodiments of the present disclosure, a viral expression construct may be used for the production of AAV particles in insect cells. In certain embodiments, modifications may be made to the wild type AAV sequences of the capsid and/or rep genes, for example to improve attributes of the viral particle, such as increased infectivity or specificity, or to enhance production yields.

In certain embodiments, a VP-coding region encodes one or more AAV capsid proteins of a specific AAV serotype. The AAV serotypes for VP-coding regions can be the same or different. In certain embodiments, a VP-coding region can be codon optimized. In certain embodiments, a VP-coding region or nucleotide sequence can be codon optimized for a mammal cell. In certain embodiments, a VP-coding region or nucleotide sequence can be codon optimized for an insect cell. In certain embodiments, a VP-coding region or nucleotide sequence can be codon optimized for a Spodoptera frugiperda cell. In certain embodiments, a VP-coding region or nucleotide sequence can be codon optimized for Sf9 or Sf21 cell lines.

In certain embodiments, a nucleotide sequence encoding one or more VP capsid proteins can be codon optimized to have a nucleotide homology with the reference nucleotide sequence of less than 100%. In certain embodiments, the nucleotide homology between the codon-optimized VP nucleotide sequence and the reference VP nucleotide sequence is less than 100%, less than 99%, less than 98%, less than 97%, less than 96%, less than 95%, less than 94%, less than 93%, less than 92%, less than 91%, less than 90%, less than 89%, less than 88%, less than 87%, less than 86%, less than 85%, less than 84%, less than 83%, less than 82%, less than 81%, less than 80%, less than 78%, less than 76%, less than 74%, less than 72%, less than 70%, less than 68%, less than 66%, less than 64%, less than 62%, less than 60%, less than 55%, less than 50%, and less than 40%.

In certain embodiments, viral expression constructs may be used that are taught in U.S. Pat. Nos. 8,512,981, 8,163,543, 8,697,417, 8,642,314, U.S. Patent Publication Nos. US20130296532, US20110119777, US20110136227, US20110171262, US20130023034, International Patent Application Nos. PCT/NL2008/050613, PCT/NL2009/050076, PCT/NL2009/050352, PCT/NL2011/050170, PCT/NL2012/050619 and U.S. patent application Ser. No. 14/149,953, the contents of each of which are herein incorporated by reference in their entirety.

In certain embodiments, the viral expression construct of the present disclosure may be derived from viral expression constructs taught in U.S. Pat. Nos. 6,468,524, 6,984,517, 7,479,554, 6,855,314, 7,271,002, 6,723,551, US Patent Publication No. 20140107186, U.S. patent application Ser. No. 09/717,789, U.S. Ser. No. 11/936,394, U.S. Ser. No. 14/004,379, European Patent Application EP1082413, EP2500434, EP 2683829, EP1572893 and International Patent Application PCT/US99/11958, PCT/US01/09123, PCT/EP2012/054303, and PCT/US2002/035829 the contents of each of which are herein incorporated by reference in its entirety.

In certain embodiments, the viral expression construct may include sequences from Simian species. In certain embodiments, the viral expression construct may contain sequences, including but not limited to capsid and rep sequences from International Patent Applications PCT/US1997/015694, PCT/US2000/033256, PCT/US2002/019735, PCT/US2002/033645, PCT/US2008/013067, PCT/US2008/013066, PCT/US2008/013065, PCT/US2009/062548, PCT/US2009/001344, PCT/US2010/036332, PCT/US2011/061632, PCT/US2013/041565. U.S. application Ser. No. 13/475,535, U.S. Ser. No. 13/896,722, U.S. Ser. No. 10/739,096. U.S. Ser. No. 14/073,979, US Patent Publication Nos. US20010049144, US20120093853, US20090215871, US20040136963, US20080219954, US20040171807, US20120093778, US20080090281, US20050069866, US20100260799. US20100247490, US20140044680, US20100254947, US20110223135, US20130309205, US20120189582, US20130004461, US20130315871, U.S. Pat. Nos. 6,083,716, 7,838,277, 7,344,872, 8,603,459, 8,105,574, 7,247,472, 8,231,880, 8,524,219, 8,470,310, European Patent Application Nos. EP2301582, EP2286841, EP1944043, EP1453543, EP1409748, EP2463362, EP2220217, EP2220241, EP2220242, EP2350269, EP2250255, EP2435559, EP2643465, EP1409748, EP2325298, EP1240345, the contents of each of which is herein incorporated by reference in its entirety.

In certain embodiments, viral expression constructs of the present disclosure may include one or more nucleotide sequence from one or more viral construct described in in International Application No. PCT/US2002/025096, PCT/US2002/033629, PCT/US2003/012405. US application No. U.S. Ser. No. 10/291,583, U.S. Ser. No. 10/420,284, U.S. Pat. No. 7,319,002, US Patent Publication No. US20040191762, US20130045186, US20110263027, US20110151434, US20030138772, US20030207259, European Application No. EP2338900, EP1456419, EP1310571, EP1359217, EP1427835, EP2338900, EP1456419, EP1310571, EP1359217 and U.S. Pat. Nos. 7,235,393 and 8,524,446.

In certain embodiments, the viral expression constructs of the present disclosure may include sequences or compositions described in International Patent Application No. PCT/US1999/025694. PCT/US1999/010096, PCT/US2001/013000, PCT/US2002/25976, PCT/US2002/033631, PCT/US2002/033630, PCT/US2009/041606, PCT/US2012/025550, U.S. Pat. Nos. 8,637,255, 8,637,255, 7,186,552, 7,105,345, 6,759,237, 7,056,502, 7,198,951, 8,318,480, 7,790,449, 7,282,199, US Patent Publication No. US20130059289, US20040057933, US20040057932. US20100278791, US20080050345, US20080050343, US20080008684, US20060204479, US20040057931. US20040052764, US20030013189, US20090227030, US20080075740, US20080075737, US20030228282, US20130323226, US20050014262, US patent application Ser. No. 14/136,331, U.S. Ser. No. 09/076,369, U.S. Ser. No. 10/738,609, European Application No. EP2573170, EP 1127150, EP2341068, EP1845163, EP1127150, EP1078096, EP1285078, EP1463805, EP2010178940, US20140004143, EP2359869, EP1453547, EP2341068, and EP2675902, the contents of each of which are herein incorporated by reference in their entirety.

In certain embodiments, viral expression construct of the present disclosure may include one or more nucleotide sequence from one or more of those described in U.S. Pat. Nos. 7,186,552, 7,105,345, 6,759,237, 7,056,502, 7,198,951, 8,318,480, 7,790,449, 7,282,199, US Patent Publication No. US20130059289, US20040057933, US20040057932, US20100278791, US20080050345, US20080050343, US20080008684, US20060204479, US20040057931, US20140004143, US20090227030, US20080075740, US20080075737, US20030228282, US20040052764, US20030013189, US20050014262, US20130323226, US patent application Ser. No. 14/136,331. U.S. Ser. No. 10/738,609, European Patent Application Nos. EP1127150, EP2341068, EP1845163, EP1127150, EP1078096, EP1285078, EP2573170, EP1463805, EP2675902, EP2359869, EP1453547, EP2341068, the contents of each of which are incorporated herein by reference in their entirety.

In certain embodiments, the viral expression constructs of the present disclosure may include constructs of modified AAVs, as described in International Patent Application No. PCT/US1995/014018, PCT/US2000/026449, PCT/US2004/028817, PCT/US2006/013375, PCT/US2007/010056, PCT/US2010/032158, PCT/US2010/050135, PCT/US2011/033596, U.S. patent application Ser. No. 12/473,917. U.S. Ser. No. 08/331,384, U.S. Ser. No. 09/670,277, U.S. Pat. Nos. 5,871,982, 5,856,152, 6,251,677, 6,387,368, 6,399,385, 7,906,111, European Patent Application No. EP2000103600, European Patent Publication No. EP797678, EP1046711, EP1668143, EP2359866, EP2359865, EP2357010, EP1046711. EP1218035, EP2345731, EP2298926, EP2292780, EP2292779, EP1668143, US20090197338, EP2383346, EP2359867, EP2359866, EP2359865, EP2357010, EP1866422, US20090317417, EP2016174, US Patent Publication Nos. US20110236353, US20070036760, US20100186103, US20120137379, and US20130281516, the contents of each of which are herein incorporated by reference in their entirety.

In certain embodiments, the viral expression constructs of the present disclosure may include one or more constructs described in International Application Nos. PCT/US1999/004367, PCT/US2004/010965, PCT/US2005/014556, PCT/US2006/009699, PCT/US2010/032943, PCT/US2011/033628, PCT/US2011/033616, PCT/US2012/034355, U.S. Pat. No. 8,394,386, EP1742668, US Patent Publication Nos. US20080241189, US20120046349, US20130195801, US20140031418, EP2425000, US20130101558, EP1742668, EP2561075, EP2561073, EP2699688, the contents of each of which is herein incorporated by reference in its entirety.

### Payload Construct: General

AAV particles of the present disclosure can include, or be produced using, at least one payload construct which includes at least one payload region. As used herein, “payload” or “payload region” refers to one or more polynucleotides or polynucleotide regions encoded by or within a viral genome (e.g., payload sequence), or an expression product of such polynucleotide or polynucleotide region (e.g., a transgene, a polynucleotide encoding a polypeptide or multi-polypeptide or a modulatory nucleic acid or regulatory nucleic acid).

The payload region may be constructed in such a way as to reflect a region similar to or mirroring the natural organization of an mRNA.

The payload region may include a combination of coding and non-coding nucleic acid sequences. In certain embodiments, the AAV payload region may encode a coding or non-coding RNA, or a combination thereof.

The payload region may also optionally comprise one or more functional or regulatory elements to facilitate transcriptional expression and/or polypeptide translation. The nucleic acid sequences and polypeptides disclosed herein may be engineered to contain modular elements and/or sequence motifs assembled to enable expression of the modulatory polynucleotides and/or modulatory polynucleotide-based compositions. In some embodiments, the nucleic acid sequence comprising the payload region may comprise one or more of a promoter region, an intron, a Kozak sequence, an enhancer or a polyadenylation sequence. Payload regions disclosed herein typically encode at least one sense and antisense sequence, an siRNA-based compositions, or fragments of the foregoing in combination with each other or in combination with other polypeptide moieties.

The payload region(s) within the viral genome of an AAV particle disclosure may be delivered to one or more target cells, tissues, organs or organisms.

In certain embodiments, the payload region may be located within a viral genome, such as the viral genome of a payload construct. At the 5′ and/or the 3′ end of the payload region there may be at least one inverted terminal repeat (ITR). Within the payload region, there may be a promoter region, an intron region and a coding region.

In certain embodiments, the AAV particles of the present disclosure are useful in the field of medicine for the treatment, prophylaxis, palliation, or amelioration of diseases and/or disorders, including neurological diseases and/or disorders.

In certain embodiments, the AAV particles of the present disclosure are useful in the field of medicine for the treatment, prophylaxis, palliation or amelioration of Friedreich's ataxia, or any disease stemming from a loss or partial loss of frataxin protein.

In certain embodiments, the AAV particles of the present disclosure are useful in the field of medicine for the treatment, prophylaxis, palliation, or amelioration of Parkinson's Disease.

In certain embodiments, the AAV particles of the present disclosure are useful in the field of medicine for the treatment, prophylaxis, palliation, or amelioration of Amyotrophic lateral sclerosis.

In certain embodiments, the AAV particles of the present disclosure are useful in the field of medicine for the treatment, prophylaxis, palliation, or amelioration of Huntington's Disease.

In certain embodiments, the payload region of the AAV particle includes one or more nucleic acid sequences encoding a polypeptide or protein of interest.

In certain embodiments, the AAV particle includes a viral genome with a payload region comprising nucleic acid sequences encoding more than one polypeptide of interest. In certain embodiments, a viral genome encoding one or more polypeptides may be replicated and packaged into a viral particle. A target cell transduced with a viral particle comprising the vector genome may express each of the one or more polypeptides in the single target cell.

Where the AAV particle payload region encodes a polypeptide, the polypeptide may be a peptide, polypeptide, or protein. As a non-limiting example, the payload region may encode at least one therapeutic protein of interest. The AAV viral genomes encoding polypeptides described herein may be useful in the fields of human disease, viruses, infections veterinary applications and a variety of in vivo and in vitro settings.

In certain embodiments, administration of the formulated AAV particles (which include the viral genome) to a subject will increase the expression of a protein in a subject. In certain embodiments, the increase of the expression of the protein will reduce the effects and/or symptoms of a disease or ailment associated with the polypeptide encoded by the payload.

In certain embodiments, the formulated AAV particles of the present disclosure may be used to reduce the decline of functional capacity and activities of daily living as measured by a standard evaluation system such as, but not limited to, the total functional capacity (TFC) scale.

In certain embodiments, the AAV particle includes a viral genome with a payload region comprising a nucleic acid sequence encoding a protein of interest (i.e., a payload protein, therapeutic protein).

In certain embodiments, the payload region comprises a nucleic acid sequence encoding a protein including but not limited to an antibody, Aromatic L-Amino Acid Decarboxylase (AADC), ApoE2, Frataxin, survival motor neuron (SMN) protein, glucocerebrosidase, N-sulfoglucosamine sulfohydrolase, N-acetyl-alpha-glucosaminidase, iduronate 2-sulfatase, alpha-L-iduronidase, palmitoyl-protein thioesterase 1, tripeptidyl peptidase 1, battenin, CLN5, CLN6 (linclin), MFSD8, CLN8, aspartoacylase (ASPA), progranulin (GRN), MeCP2, beta-galactosidase (GLB1) and/or gigaxonin (GAN).

In certain embodiments, the AAV particle includes a viral genome with a payload region comprising a nucleic acid sequence encoding AADC or any other payload known in the art for treating Parkinson's disease. As a non-limiting example, the payload may include a sequence such as NM_001082971.1 (GI: 132814447). NM_000790.3 (GI: 132814459), NM_001242886.1 (GI: 338968913), NM_001242887.1 (GI: 338968916), NM_001242888.1 (GI: 338968918), NM_001242889.1 (GI: 338968920), NM_001242890.1 (GI: 338968922) and fragment or variants thereof.

In certain embodiments, the AAV particle includes a viral genome with a payload region comprising a nucleic acid sequence encoding frataxin or any other payload known in the art for treating Friedreich's Ataxia. As a non-limiting example, the payload may include a sequence such as NM_000144.4 (GI: 239787167), NM_181425.2 (GI: 239787185), NM_001161706.1 (GI: 239787197) and fragment or variants thereof.

In certain embodiments, the AAV particle includes a viral genome with a payload region comprising a nucleic acid sequence encoding SMN or any other payload known in the art for treating spinal muscular atrophy (SMA). As a non-limiting example, the payload may include a sequence such as NM_001297715.1 (GI: 663070993), NM_000344.3 (GI: 196115055), NM_022874.2 (GI: 196115040) and fragment or variants thereof.

In certain embodiments, the AAV particle includes a viral genome with a payload region comprising a nucleic acid sequence encoding any of the disease-associated proteins (and fragment or variants thereof) described in U. S. Patent publication No. 20180258424; the content of which is herein incorporated by reference in its entirety.

In certain embodiments, the AAV particle includes a viral genome with a payload region comprising a nucleic acid sequence encoding any of the disease-associated proteins (and fragment or variants thereof) described in any one of the following International Publications: WO2016073693, WO2017023724, WO2018232055, WO2016077687, WO2016077689, WO2018204786. WO2017201258. WO2017201248. WO2018204803. WO2018204797, WO2017189959, WO2017189963, WO2017189964, WO2015191508, WO2016094783, WO20160137949, WO2017075335; the contents of which are each herein incorporated by reference in their entirety.

### Payload: Modulatory Polynucleotides Targeting a Gene of Interest

### General

The present disclosure comprises the use of formulated AAV particles whose vector genomes encode modulatory polynucleotides, e.g., RNA or DNA molecules. As used herein, a “modulatory polynucleotide” is any nucleic acid sequence(s) which functions to modulate (either increase or decrease) the level or amount of a target gene, e.g., mRNA or protein levels. Accordingly, the present disclosure provides vector genomes encoding polynucleotides that can be processed into RNA molecules which can target a gene of interest inside of a cell such RNA molecules include, but are not limited to, double stranded RNA (dsRNA), small interfering RNA (siRNA), microRNA (miRNA), pre-miRNA, or other RNAi agents. The present disclosure also provides methods of their use for inhibiting gene expression and protein production of an allele of the gene of interest, for treating diseases, disorders, and/or conditions.

In certain embodiments, the AAV particle includes a viral genome with a payload region comprising a nucleic acid sequence encoding or including one or more modulatory polynucleotides. In certain embodiments, the AAV particle includes a viral genome with a payload region comprising a nucleic acid sequence encoding a modulatory polynucleotide of interest. In certain embodiments of the present disclosure, modulatory polynucleotides, e.g., RNA or DNA molecules, are presented as therapeutic agents. RNA interference mediated gene silencing can specifically inhibit targeted gene expression.

In certain embodiments, the payload region comprises a nucleic acid sequence encoding a modulatory polynucleotide which interferes with a target gene expression and/or a target protein production. In certain embodiments, the gene expression or protein production to be inhibited/modified may include but are not limited to superoxide dismutase 1 (SOD1), chromosome 9 open reading frame 72 (C9ORF72), TAR DNA binding protein (TARDBP), ataxin-3 (ATXN3), huntingtin (I-HTT), amyloid precursor protein (APP), apolipoprotein E (ApoE), microtubule-associated protein tau (MAPT), alpha-synuclein (SNCA), voltage-gated sodium channel alpha subunit 9 (SCN9A), and/or voltage-gated sodium channel alpha subunit 10 (SCN10A).

The present disclosure provides small interfering RNA (siRNA) duplexes (and modulatory polynucleotides encoding them) that target SOD1 mRNA to interfere with the gene expression and/or protein production of SOD1. The present disclosure also provides methods of their use for inhibiting gene expression and protein production of an allele of SOD1, for treating amyotrophic lateral sclerosis (ALS). In certain embodiments, the siRNA duplexes of the present disclosure may target SOD1 along any segment of the respective nucleotide sequence. In certain embodiments, the siRNA duplexes of the present disclosure may target SOD1 at the location of a SNP or variant within the nucleotide sequence.

The present disclosure provides small interfering RNA (siRNA) duplexes (and modulatory polynucleotides encoding them) that target HTT mRNA to interfere with the gene expression and/or protein production of HTT. The present disclosure also provides methods of their use for inhibiting gene expression and protein production of an allele of HTT, for treating Huntington's disease (HD). In certain embodiments, the siRNA duplexes of the present disclosure may target HTT along any segment of the respective nucleotide sequence. In certain embodiments, the siRNA duplexes of the present disclosure may target HTT at the location of a SNP or variant within the nucleotide sequence.

In certain embodiments, the AAV particle includes a viral genome with a payload region comprising a nucleic acid sequence encoding any of the modulatory polynucleotides, RNAi molecules, siRNA molecules, dsRNA molecules, and/or RNA duplexes described in any one of the following International Publications: WO2016073693, WO2017023724, WO2018232055, WO2016077687, WO2016077689, WO2018204786, WO2017201258, WO2017201248, WO2018204803, WO2018204797, WO2017189959, WO2017189963, WO2017189964, WO2015191508, WO2016094783, WO20160137949, WO2017075335; the contents of which are each herein incorporated by reference in their entirety.

In certain embodiments, a nucleic acid sequence encoding such siRNA molecules, or a single strand of the siRNA molecules, is inserted into adeno-associated viral vectors and introduced into cells, specifically cells in the central nervous system.

AAV particles have been investigated for siRNA delivery because of several unique features. Non-limiting examples of the features include (i) the ability to infect both dividing and non-dividing cells; (ii) a broad host range for infectivity, including human cells; (iii) wild-type AAV has not been associated with any disease and has not been shown to replicate in infected cells; (iv) the lack of cell-mediated immune response against the vector and (v) the non-integrative nature in a host chromosome thereby reducing potential for long-term expression. Moreover, infection with AAV particles has minimal influence on changing the pattern of cellular gene expression (Stilwell and Samulski et al., Biotechniques, 2003, 34, 148).

In certain embodiments, the encoded siRNA duplex of the present disclosure contains an antisense strand and a sense strand hybridized together, wherein the antisense strand is complementary to the nucleic acid sequence of the targeted gene of interest, and wherein the sense strand is homologous to the nucleic acid sequence of the targeted gene of interest. In other aspects, there are 0, 1 or 2 nucleotide overhangs at the 3′end of each strand.

According to the present disclosure, each strand of the siRNA duplex targeting the gene of interest can be about 19 to 25, 19 to 24 or 19 to 21 nucleotides in length, such as about 19 nucleotides, 20 nucleotides, 21 nucleotides, 22 nucleotides, 23 nucleotides, 24 nucleotides, or 25 nucleotides in length.

In certain embodiments, an siRNA or dsRNA includes at least two sequences that are complementary to each other. The dsRNA includes a sense strand having a first sequence and an antisense strand having a second sequence. The antisense strand includes a nucleotide sequence that is substantially complementary to at least part of an mRNA encoding a gene of interest, and the region of complementarity is 30 nucleotides or less, and at least 15 nucleotides in length. Generally, the dsRNA is 19 to 25, 19 to 24 or 19 to 21 nucleotides in length. In certain embodiments, the dsRNA is from about 15 to about 25 nucleotides in length, and in certain embodiments the dsRNA is from about 25 to about 30 nucleotides in length.

The dsRNA encoded in an expression vector upon contacting with a cell expressing protein encoded by the gene of interest, inhibits the expression of protein encoded by the gene of interest by at least 10%, at least 20%, at least 25%, at least 30%, at least 35%, at least 40%, at least 50%, at least 60%, at least 70%, at least 80%, or more, when assayed by methods known in the art or a method as described herein.

According to the present disclosure, the siRNA molecules are designed and tested for their ability in reducing mRNA levels in cultured cells.

In certain embodiments, the siRNA molecules are designed and tested for their ability in reducing levels of the gene of interest in cultured cells.

The present disclosure also provides pharmaceutical compositions comprising at least one siRNA duplex targeting the gene of interest and a pharmaceutically acceptable carrier. In some aspects, the siRNA duplex is encoded by a vector genome in an AAV particle.

In certain embodiments, the present disclosure provides methods for inhibiting/silencing gene expression in a cell. In some aspects, the inhibition of gene expression refers to an inhibition by at least about 20%, such as by at least about 30%, 40%, 50%, 60%, 70%, 80%, 85%, 90%, 95% and 100%, or at least 20-30%, 2040%, 20-50%, 20-60%, 20-70%, 20-80%, 20-90%, 20-95%, 20-100%, 30-40%, 35-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. Accordingly, the protein product of the targeted gene may be inhibited by at least about 20%, such as by at least about 30%, 40%, 50%, 60%, 70%, 80%, 85%, 90%, 95% and 100%, or at least 20-30%, 2040%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%.

In certain embodiments, the encoded siRNA duplexes may be used to reduce the expression of protein encoded by the gene of interest by at least about 20%, 30%, 40%, 50%, 60%, 70%, 80%, 85%, 90%, 95% and 100%, or at least 20-30%, 2040%, 20-50%, 20-60%, 20-70%, 20-80%, 20-90%, 20-95%, 20-100%, 30-40%, 3540%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. As a non-limiting example, the expression of protein may be reduced 50-90%. As a non-limiting example, the expression of protein may be reduced 30-70%. As a non-limiting example, the expression of protein may be reduced 40-70%.

In certain embodiments, the encoded siRNA duplexes may be used to reduce the expression of mRNA transcribed from the gene of interest by at least about 20%, 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 35-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. As a non-limiting example, the expression of mRNA expression may be reduced 50-90%.

In certain embodiments, the encoded siRNA duplexes may be used to reduce the expression of protein encoded by the gene of interest and/or transcribed mRNA in at least one region of the CNS. The expression of protein and/or mRNA is reduced by at least about 20%, 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 35-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in at least one region of the CNS. As a non-limiting example, the region is the neurons (e.g., cortical neurons).

In certain embodiments, the formulated AAV particles comprising such encoded siRNA molecules may be introduced directly into the central nervous system of the subject, for example, by infusion into the putamen.

In certain embodiments, the formulated AAV particles comprising such encoded siRNA molecules may be introduced directly into the central nervous system of the subject, for example, by infusion into the thalamus of a subject.

In certain embodiments, the formulated AAV particles comprising such encoded siRNA molecules may be introduced directly into the central nervous system of the subject, for example, by infusion into the white matter of a subject.

In certain embodiments, the formulated AAV particles comprising such encoded siRNA molecules may be introduced to the central nervous system of the subject, for example, by intravenous administration to a subject.

In certain embodiments, the pharmaceutical composition of the present disclosure is used as a solo therapy. In certain embodiments, the pharmaceutical composition of the present disclosure is used in combination therapy. The combination therapy may be in combination with one or more neuroprotective agents such as small molecule compounds, growth factors and hormones which have been tested for their neuroprotective effect on motor neuron degeneration.

siRNA Molecules

The payloads of the formulated AAV particles of the present disclosure may encode one or more agents which are subject to RNA interference (RNAi) induced inhibition of gene expression. Provided herein are encoded siRNA duplexes or encoded dsRNA that target a gene of interest (referred to herein collectively as “siRNA molecules”). Such siRNA molecules, e.g., encoded siRNA duplexes, encoded dsRNA or encoded siRNA or dsRNA precursors can reduce or silence gene expression in cells, for example, astrocytes or microglia, cortical, hippocampal, entorhinal, thalamic, sensory, or motor neurons.

RNAi (also known as post-transcriptional gene silencing (PTGS), quelling, or co-suppression) is a post-transcriptional gene silencing process in which RNA molecules, in a sequence specific manner, inhibit gene expression, typically by causing the destruction of specific mRNA molecules. The active components of RNAi are short/small double stranded RNAs (dsRNAs), called small interfering RNAs (siRNAs), that typically contain 15-30 nucleotides (e.g., 19 to 25, 19 to 24 or 19-21 nucleotides) and 2-nucleotide 3′ overhangs and that match the nucleic acid sequence of the target gene. These short RNA species may be naturally produced in vivo by Dicer-mediated cleavage of larger dsRNAs and they are functional in mammalian cells.

In some embodiments, the modulatory polynucleotides of the vector genome may comprise at least one nucleic acid sequence encoding at least one siRNA molecule. The nucleic acid sequence may, independently if there is more than one, encode 1, 2, 3, 4, 5, 6, 7, 8, 9, or more than 9 siRNA molecules.

Naturally expressed small RNA molecules, known as microRNAs (miRNAs), elicit gene silencing by regulating the expression of mRNAs. The miRNAs containing RNA Induced Silencing Complex (RISC) targets mRNAs presenting a perfect sequence complementarity with nucleotides 2-7 in the 5′ region of the miRNA which is called the seed region, and other base pairs with its 3′ region. miRNA mediated down regulation of gene expression may be caused by cleavage of the target mRNAs, translational inhibition of the target mRNAs, or mRNA decay. miRNA targeting sequences are usually located in the 3′ UTR of the target mRNAs. A single miRNA may target more than 100 transcripts from various genes, and one mRNA may be targeted by different miRNAs.

siRNA duplexes or dsRNA targeting a specific mRNA may be designed as a payload of an AAV particle and introduced into cells for activating RNAi processes. Elbashir et al. demonstrated that 21-nucleotide siRNA duplexes (termed small interfering RNAs) were capable of effecting potent and specific gene knockdown without inducing immune response in mammalian cells (Elbashir S M et al., Nature, 2001, 411, 494-498). Since this initial report, post-transcriptional gene silencing by siRNAs quickly emerged as a powerful tool for genetic analysis in mammalian cells and has the potential to produce novel therapeutics.

The siRNA duplex comprised of a sense strand homologous to the target mRNA and an antisense strand that is complementary to the target mRNA offers much more advantage in terms of efficiency for target RNA destruction compared to the use of the single strand (ss)-siRNAs (e.g., antisense strand RNA or antisense oligonucleotides). In many cases it requires higher concentration of the ss-siRNA to achieve the effective gene silencing potency of the corresponding duplex.

Introduction into Cells—AAV Particles

The encoded siRNA molecules (e.g., siRNA duplexes) of the present disclosure may be introduced into cells by being encoded by the vector genome of an AAV particle. These AAV particles are engineered and optimized to facilitate the entry into cells that are not readily amendable to transfection/transduction. Also, some synthetic viral vectors possess an ability to integrate the shRNA into the cell genome, thereby leading to stable siRNA expression and long-term knockdown of a target gene. In this manner, viral vectors are engineered as vehicles for specific delivery while lacking the deleterious replication and/or integration features found in wild-type virus.

In certain embodiments, the encoded siRNA molecule is introduced into a cell by transfecting, infecting, or transducing the cell with an AAV particle comprising nucleic acid sequences capable of producing the siRNA molecule when transcribed in the cell. In certain embodiments, the siRNA molecule is introduced into a cell by injecting into the cell or tissue an AAV particle comprising a nucleic acid sequence capable of producing the siRNA molecule when transcribed in the cell.

In certain embodiments, prior to transfection/transduction, an AAV particle comprising a nucleic acid sequence encoding the siRNA molecules of the present disclosure may be transfected into cells.

Other methods for introducing AAV particles comprising the nucleic acid sequence for the siRNA molecules described herein may include photochemical internalization as described in U. S. Patent publication No. 20120264807; the content of which is herein incorporated by reference in its entirety.

In certain embodiments, the formulations described herein may contain at least one AAV particle comprising the nucleic acid sequence encoding the siRNA molecules described herein. In certain embodiments, the siRNA molecules may target the gene of interest at one target site. In another embodiment, the formulation comprises a plurality of AAV particles, each AAV particle comprising a nucleic acid sequence encoding a siRNA molecule targeting the gene of interest at a different target site. The gene of interest may be targeted at 2, 3, 4, 5 or more than 5 sites.

In certain embodiments, the AAV particles from any relevant species, such as, but not limited to, human, pig, dog, mouse, rat, or monkey may be introduced into cells.

In certain embodiments, the formulated AAV particles may be introduced into cells or tissues which are relevant to the disease to be treated.

In certain embodiments, the formulated AAV particles may be introduced into cells which have a high level of endogenous expression of the target sequence.

In another embodiment, the formulated AAV particles may be introduced into cells which have a low level of endogenous expression of the target sequence.

In certain embodiments, the cells may be those which have a high efficiency of AAV transduction.

In certain embodiments, formulated AAV particles comprising a nucleic acid sequence encoding the siRNA molecules of the present disclosure may be used to deliver siRNA molecules to the central nervous system (e.g., U.S. Pat. No. 6,180,613; the contents of which is herein incorporated by reference in its entirety).

In some aspects, the formulated AAV particles comprising a nucleic acid sequence encoding the siRNA molecules of the present disclosure may further comprise a modified capsid including peptides from non-viral origin. In other aspects, the AAV particle may contain a CNS specific chimeric capsid to facilitate the delivery of encoded siRNA duplexes into the brain and the spinal cord. For example, an alignment of cap nucleotide sequences from AAV variants exhibiting CNS tropism may be constructed to identify variable region (VR) sequence and structure.

In certain embodiments, the formulated AAV particle comprising a nucleic acid sequence encoding the siRNA molecules of the present disclosure may encode siRNA molecules which are polycistronic molecules. The siRNA molecules may additionally comprise one or more linkers between regions of the siRNA molecules.

In certain embodiments, a formulated AAV particle may comprise at least one of the modulatory polynucleotides encoding at least one of the siRNA sequences or duplexes described herein.

In certain embodiments, an expression vector may comprise, from ITR to ITR recited 5′ to 3′, an ITR, a promoter, an intron, a modulatory polynucleotide, a polyA sequence and an ITR.

In certain embodiments, the encoded siRNA molecule may be located downstream of a promoter in an expression vector such as, but not limited to, CMV, U6, H1, CBA or a CBA promoter with a SV40 intron. Further, the encoded siRNA molecule may also be located upstream of the polyadenylation sequence in an expression vector. As a non-limiting example, the encoded siRNA molecule may be located within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 nucleotides downstream from the promoter and/or upstream of the polyadenylation sequence in an expression vector. As another non-limiting example, the encoded siRNA molecule may be located within 1-5, 1-10, 1-15, 1-20, 1-25, 1-30, 5-10, 5-15, 5-20, 5-25, 5-30, 10-15, 10-20, 10-25, 10-30, 15-20, 15-25, 15-30, 20-25, 20-30 or 25-30 nucleotides downstream from the promoter and/or upstream of the polyadenylation sequence in an expression vector. As a non-limiting example, the encoded siRNA molecule may be located within the first 1%, 2%, 3%, 4%, 5%, 6%, 7%, 8%, 9%, 10%, 15%, 20%, 25% or more than 25% of the nucleotides downstream from the promoter and/or upstream of the polyadenylation sequence in an expression vector. As another non-limiting example, the encoded siRNA molecule may be located with the first 1-5%, 1-10%, 1-15%, 1-20%, 1-25%, 5-10%, 5-15%, 5-20%, 5-25%, 10-15%, 10-20%, 10-25%, 15-20%, 15-25%, or 20-25% downstream from the promoter and/or upstream of the polyadenylation sequence in an expression vector.

In certain embodiments, the encoded siRNA molecule may be located upstream of the polyadenylation sequence in an expression vector. Further, the encoded siRNA molecule may be located downstream of a promoter such as, but not limited to, CMV, U6, CBA or a CBA promoter with a SV40 intron in an expression vector. As a non-limiting example, the encoded siRNA molecule may be located within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 nucleotides downstream from the promoter and/or upstream of the polyadenylation sequence in an expression vector. As another non-limiting example, the encoded siRNA molecule may be located within 1-5, 1-10, 1-15, 1-20, 1-25, 1-30, 5-10, 5-15, 5-20, 5-25, 5-30, 10-15, 10-20, 10-25, 10-30, 15-20, 15-25, 15-30, 20-25, 20-30 or 25-30 nucleotides downstream from the promoter and/or upstream of the polyadenylation sequence in an expression vector. As a non-limiting example, the encoded siRNA molecule may be located within the first 1%, 2%, 3%, 4%, 5%, 6%, 7%, 8%, 9%, 10%, 15%, 20%, 25% or more than 25% of the nucleotides downstream from the promoter and/or upstream of the polyadenylation sequence in an expression vector. As another non-limiting example, the encoded siRNA molecule may be located with the first 1-5%, 1-10%, 1-15%, 1-20%, 1-25%, 5-10%, 5-15%, 5-20%, 5-25%, 10-15%, 10-20%, 10-25%, 15-20%, 15-25%, or 20-25% downstream from the promoter and/or upstream of the polyadenylation sequence in an expression vector.

In certain embodiments, the encoded siRNA molecule may be located in a scAAV.

In certain embodiments, the encoded siRNA molecule may be located in an ssAAV.

In certain embodiments, the encoded siRNA molecule may be located near the 5′ end of the flip ITR in an expression vector. In another embodiment, the encoded siRNA molecule may be located near the 3′ end of the flip ITR in an expression vector. In yet another embodiment, the encoded siRNA molecule may be located near the 5′ end of the flop ITR in an expression vector. In yet another embodiment, the encoded siRNA molecule may be located near the 3′ end of the flop ITR in an expression vector. In certain embodiments, the encoded siRNA molecule may be located between the 5′ end of the flip ITR and the 3′ end of the flop ITR in an expression vector. In certain embodiments, the encoded siRNA molecule may be located between (e.g., half-way between the 5′ end of the flip ITR and 3′ end of the flop ITR or the 3′ end of the flop ITR and the 5′ end of the flip ITR), the 3′ end of the flip ITR and the 5′ end of the flip ITR in an expression vector. As a non-limiting example, the encoded siRNA molecule may be located within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 nucleotides downstream from the 5′ or 3′ end of an ITR (e.g., Flip or Flop ITR) in an expression vector. As a non-limiting example, the encoded siRNA molecule may be located within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 nucleotides upstream from the 5′ or 3′ end of an ITR (e.g., Flip or Flop ITR) in an expression vector. As another non-limiting example, the encoded siRNA molecule may be located within 1-5, 1-10, 1-15, 1-20, 1-25, 1-30, 5-10, 5-15, 5-20, 5-25, 5-30, 10-15, 10-20, 10-25, 10-30, 15-20, 15-25, 15-30, 20-25, 20-30 or 25-30 nucleotides downstream from the 5′ or 3′ end of an ITR (e.g., Flip or Flop ITR) in an expression vector. As another non-limiting example, the encoded siRNA molecule may be located within 1-5, 1-10, 1-15, 1-20, 1-25, 1-30, 5-10, 5-15, 5-20, 5-25, 5-30, 10-15, 10-20, 10-25, 10-30, 15-20, 15-25, 15-30, 20-25, 20-30 or 25-30 upstream from the 5′ or 3′ end of an ITR (e.g., Flip or Flop ITR) in an expression vector. As a non-limiting example, the encoded siRNA molecule may be located within the first 1%, 2%, 3%, 4%, 5%, 6%, 7%, 8%, 9%, 10%, 15%, 20%, 25% or more than 25% of the nucleotides upstream from the 5′ or 3′ end of an ITR (e.g., Flip or Flop ITR) in an expression vector. As another non-limiting example, the encoded siRNA molecule may be located with the first 1-5%, 1-10%, 1-15%, 1-20%, 1-25%, 5-10%, 5-15%, 5-20%, 5-25%, 10-15%, 10-20%, 10-25%, 15-20%, 15-25%, or 20-25% downstream from the 5′ or 3′ end of an ITR (e.g., Flip or Flop ITR) in an expression vector.

In certain embodiments, AAV particle comprising the nucleic acid sequence for the siRNA molecules of the present disclosure may be formulated for CNS delivery. Agents that cross the brain blood barrier may be used. For example, some cell penetrating peptides that can target siRNA molecules to the brain blood barrier endothelium may be used to formulate the siRNA duplexes targeting the gene of interest.

In certain embodiments, the formulated AAV particle comprising a nucleic acid sequence encoding the siRNA molecules of the present disclosure may be administered directly to the CNS. As a non-limiting example, the vector comprises a nucleic acid sequence encoding the siRNA molecules targeting the gene of interest.

In specific embodiments, compositions of formulated AAV particles comprising a nucleic acid sequence encoding the siRNA molecules of the present disclosure may be administered in a way which facilitates the vectors or siRNA molecule to enter the central nervous system and penetrate into motor neurons.

In certain embodiments, the formulated AAV particle may be administered to a subject (e.g., to the CNS of a subject via intrathecal administration) in a therapeutically effective amount for the siRNA duplexes or dsRNA to target the motor neurons and astrocytes in the spinal cord and/or brain stem. As a non-limiting example, the siRNA duplexes or dsRNA may reduce the expression of a protein or mRNA.

### Viral Production Cells and Vectors

### Mammalian-Production System

Viral production of the present disclosure disclosed herein describes processes and methods for producing AAV particles or viral vector that contacts a target cell to deliver a payload construct, e.g., a recombinant AAV particle or viral construct, which includes a nucleotide encoding a payload molecule. The viral production cell may be selected from any biological organism, including prokaryotic (e.g., bacterial) cells, and eukaryotic cells, including, insect cells, yeast cells and mammalian cells.

In certain embodiments, the AAV particles of the present disclosure may be produced in a viral production cell that includes a mammalian cell. Viral production cells may comprise mammalian cells such as A549, WEH1, 3T3, 10T1/2, BHK, MDCK, COS 1, COS 7, BSC 1, BSC 40, BMT 10, VERO. W138, HeLa, HEK293, HEK293T (293T), Saos, C2C12, L cells, HT1080, HepG2 and primary fibroblast, hepatocyte and myoblast cells derived from mammals. Viral production cells can include cells derived from mammalian species including, but not limited to, human, monkey, mouse, rat, rabbit, and hamster or cell type, including but not limited to fibroblast, hepatocyte, tumor cell, cell line transformed cell, etc.

AAV viral production cells commonly used for production of recombinant AAV particles include, but is not limited to HEK293 cells, COS cells, C127, 3T3, CHO. HeLa cells, KB cells, BHK, and other mammalian cell lines as described in U.S. Pat. Nos. 6,156,303, 5,387,484, 5,741,683, 5,691,176, 6,428,988 and 5,688,676; U.S. patent application 2002/0081721, and International Patent Publication Nos. WO 00/47757, WO 00/24916, and WO %/17947, the contents of each of which are herein incorporated by reference in their entireties. In certain embodiments, the AAV viral production cells are trans-complementing packaging cell lines that provide functions deleted from a replication-defective helper virus, e.g., HEK293 cells or other Ea trans-complementing cells.

In certain embodiments, the packaging cell line 293-10-3 (ATCC Accession No. PTA-2361) may be used to produce the AAV particles, as described in U.S. Pat. No. 6,281,010, the contents of which are herein incorporated by reference in its entirety.

In certain embodiments, of the present disclosure a cell line, such as a HeLA cell line, for trans-complementing E1 deleted adenoviral vectors, which encoding adenovirus E1a and adenovirus E1b under the control of a phosphoglycerate kinase (PGK) promoter can be used for AAV particle production as described in U.S. Pat. No. 6,365,394, the contents of which are incorporated herein by reference in their entirety.

In certain embodiments, AAV particles are produced in mammalian cells using a triple transfection method wherein a payload construct, parvoviral Rep and parvoviral Cap and a helper construct are comprised within three different constructs. The triple transfection method of the three components of AAV particle production may be utilized to produce small lots of virus for assays including transduction efficiency, target tissue (tropism) evaluation, and stability.

AAV particles to be formulated may be produced by triple transfection or baculovirus mediated virus production, or any other method known in the art. Any suitable permissive or packaging cell known in the art may be employed to produce the vectors. In certain embodiments, trans-complementing packaging cell lines are used that provide functions deleted from a replication-defective helper virus, e.g., 293 cells or other E1a trans-complementing cells.

The gene cassette may contain some or all of the parvovirus (e.g., AAV) cap and rep genes. In certain embodiments, some or all of the cap and rep functions are provided in trans by introducing a packaging vector(s) encoding the capsid and/or Rep proteins into the cell. In certain embodiments, the gene cassette does not encode the capsid or Rep proteins. Alternatively, a packaging cell line is used that is stably transformed to express the cap and/or rep genes.

Recombinant AAV virus particles are, in certain embodiments, produced and purified from culture supernatants according to the procedure as described in US2016/0032254, the contents of which are incorporated by reference. Production may also involve methods known in the art including those using 293T cells, triple transfection or any suitable production method.

In certain embodiments, mammalian viral production cells (e.g 293T cells) can be in an adhesion/adherent state (e.g., with calcium phosphate) or a suspension state (e.g with polyethylenimine (PEI)). The mammalian viral production cell is transfected with plasmids required for production of AAV, (i.e., AAV rep/cap construct, an adenoviral helper construct, and/or ITR flanked payload construct). In certain embodiments, the transfection process can include optional medium changes (e.g., medium changes for cells in adhesion form, no medium changes for cells in suspension form, medium changes for cells in suspension form if desired). In certain embodiments, the transfection process can include transfection mediums such as DMEM or F17. In certain embodiments, the transfection medium can include serum or can be serum-free (e.g., cells in adhesion state with calcium phosphate and with serum, cells in suspension state with PEI and without serum).

Cells can subsequently be collected by scraping (adherent form) and/or pelleting (suspension form and scraped adherent form) and transferred into a receptacle. Collection steps can be repeated as necessary for full collection of produced cells. Next, cell lysis can be achieved by consecutive freeze-thaw cycles (−80C to 37C), chemical lysis (such as adding detergent triton), mechanical lysis, or by allowing the cell culture to degrade after reaching ˜0% viability. Cellular debris is removed by centrifugation and/or depth filtration. The samples are quantified for AAV particles by DNase resistant genome titration by DNA qPCR.

AAV particle titers are measured according to genome copy number (genome particles per milliliter). Genome particle concentrations are based on DNA qPCR of the vector DNA as previously reported (Clark et al. (1999) Hum. Gene Ther., 10:1031-1039; Veldwijk et al. (2002) Mol. Ther., 6:272-278).

### Insect Cells

Viral production of the present disclosure includes processes and methods for producing AAV particles or viral vectors that contact a target cell to deliver a payload construct, e.g., a recombinant viral construct, which includes a nucleotide encoding a payload molecule. In certain embodiments, the AAV particles or viral vectors of the present disclosure may be produced in a viral production cell that includes an insect cell.

Growing conditions for insect cells in culture, and production of heterologous products in insect cells in culture are well-known in the art, see U.S. Pat. No. 6,204,059, the contents of which are herein incorporated by reference in their entirety.

Any insect cell which allows for replication of parvovirus and which can be maintained in culture can be used in accordance with the present disclosure. AAV viral production cells commonly used for production of recombinant AAV particles include, but is not limited to, Spodoptera frugiperda, including, but not limited to the Sf9 or Sf21 cell lines, Drosophila cell lines, or mosquito cell lines, such as Aedes albopictus derived cell lines. Use of insect cells for expression of heterologous proteins is well documented, as are methods of introducing nucleic acids, such as vectors, e.g., insect-cell compatible vectors, into such cells and methods of maintaining such cells in culture. See, for example, Methods In Molecular Biology, ed. Richard, Humana Press, N J (1995); O'Reilly et al., Baculovirus Expression Vectors, A Laboratory Manual, Oxford Univ. Press (1994); Samulski et al., J. Vir. 63:3822-8 (1989); Kajigaya et al., Proc. Nat'l. Acad. Sci. USA 88: 4646-50 (1991); Ruffing et al., J. Vir. 66:6922-30 (1992); Kimbauer et al., Vir.219:37-44 (1996); Zhao et al., Vir. 272:382-93 (2000); and Samulski et al., U.S. Pat. No. 6,204,059, the contents of each of which are herein incorporated by reference in their entirety.

In one embodiment, the AAV particles are made using the methods described in WO2015/191508, the contents of which are herein incorporated by reference in their entirety.

In certain embodiments, insect host cell systems, in combination with baculoviral systems (e.g., as described by Luckow et al., Bio/Technology 6: 47 (1988)) may be used. In certain embodiments, an expression system for preparing chimeric peptide is Trichoplusia ni, Tn 5B1-4 insect cells/baculoviral system, which can be used for high levels of proteins, as described in U.S. Pat. No. 6,660,521, the contents of which are herein incorporated by reference in their entirety.

Expansion, culturing, transfection, infection, and storage of insect cells can be carried out in any cell culture media, cell transfection media or storage media known in the art, including Hyclone SFX Insect Cell Culture Media, Expression System ESF AF Insect Cell Culture Medium, ThermoFisher Sf900II media, ThermoFisher Sf900III media, or ThermoFisher Grace's Insect Media. Insect cell mixtures of the present disclosure can also include any of the formulation additives or elements described in the present disclosure, including (but not limited to) salts, acids, bases, buffers, surfactants (such as Poloxamer 188/Pluronic F-68), and other known culture media elements. Formulation additives can be incorporated gradually or as “spikes” (incorporation of large volumes in a short time).

### Baculovirus-Production System

In certain embodiments, processes of the present disclosure can include production of AAV particles or viral vectors in a baculoviral system using a viral expression construct and a payload construct vector. In certain embodiments, the baculoviral system includes Baculovirus expression vectors (BEVs) and/or baculovirus infected insect cells (BIICs). In certain embodiments, a viral expression construct vector and a payload construct vector of the present disclosure are each incorporated by homologous recombination (transposon donor/acceptor system) into a bacmid, also known as a baculovirus plasmid, by standard molecular biology techniques known and performed by a person skilled in the art. Transfection of separate viral replication cell populations produces two or more groups (e.g. two, three) of baculoviruses (BEVs), one or more group that includes the viral expression construct (Expression BEV), and one or more group that includes the payload construct (Payload BEV). The baculoviruses may be used to infect a viral production cell for production of AAV particles or viral vector.

In certain embodiments, the process includes transfection of a single viral replication cell population to produce a single baculovirus (BEV) group which includes both the viral expression construct and the payload construct. These baculoviruses may be used to infect a viral production cell for production of AAV particles or viral vector.

In certain embodiments, BEVs are produced using a Bacmid Transfection agent, such as Promega FuGENE HD, WFI water, or ThermoFisher Cellfectin II Reagent. In certain embodiments, BEVs are produced and expanded in viral production cells, such as an insect cell.

In certain embodiments, the method utilizes seed cultures of viral production cells that include one or more BEVs, including baculovirus infected insect cells (BIICs). The seed BIICs have been transfected/transduced/infected with an Expression BEV which includes a viral expression construct, and also a Payload BEV which includes a payload construct. In certain embodiments, the seed cultures are harvested, divided into aliquots and frozen, and may be used at a later time to initiate transfection/transduction/infection of a naïve population of production cells. In certain embodiments, a bank of seed BIICs is stored at −80° C. or in LN₂ vapor.

Baculoviruses are made of several essential proteins which are essential for the function and replication of the Baculovirus, such as replication proteins, envelope proteins and capsid proteins. The Baculovirus genome thus includes several essential-gene nucleotide sequences encoding the essential proteins. As a non-limiting example, the genome can include an essential-gene region which includes an essential-gene nucleotide sequence encoding an essential protein for the Baculovirus construct. The essential protein can include: GP64 baculovirus envelope protein, VP39 baculovirus capsid protein, or other similar essential proteins for the Baculovirus construct.

Baculovirus expression vectors (BEV) for producing AAV particles in insect cells, including but not limited to Spodoptera frugiperda (Sf9) cells, provide high titers of viral vector product. Recombinant baculovirus encoding the viral expression construct and payload construct initiates a productive infection of viral vector replicating cells. Infectious baculovirus particles released from the primary infection secondarily infect additional cells in the culture, exponentially infecting the entire cell culture population in a number of infection cycles that is a function of the initial multiplicity of infection, see Urabe, M. et al. J Virol. 2006 February; 80(4):1874-85, the contents of which are herein incorporated by reference in their entirety.

Production of AAV particles with baculovirus in an insect cell system may address known baculovirus genetic and physical instability.

In certain embodiments, the production system of the present disclosure addresses baculovirus instability over multiple passages by utilizing a titerless infected-cells preservation and scale-up system. Small scale seed cultures of viral producing cells are transfected with viral expression constructs encoding the structural and/or non-structural components of the AAV particles. Baculovirus-infected viral producing cells are harvested into aliquots that may be cryopreserved in liquid nitrogen; the aliquots retain viability and infectivity for infection of large scale viral producing cell culture Wasilko D J et al. Protein Expr Purif. 2009 June; 65(2):122-32, the contents of which are herein incorporated by reference in their entirety.

A genetically stable baculovirus may be used to produce a source of the one or more of the components for producing AAV particles in invertebrate cells. In certain embodiments, defective baculovirus expression vectors may be maintained episomally in insect cells. In such an embodiment the bacmid vector is engineered with replication control elements, including but not limited to promoters, enhancers, and/or cell-cycle regulated replication elements.

In certain embodiments, baculoviruses may be engineered with a (non-) selectable marker for recombination into the chitinase/cathepsin locus. The chia/v-cath locus is non-essential for propagating baculovirus in tissue culture, and the V-cath (EC 3.4.22.50) is a cysteine endoprotease that is most active on Arg-Arg dipeptide containing substrates. The Arg-Arg dipeptide is present in densovirus and parvovirus capsid structural proteins but infrequently occurs in dependovirus VP1.

In certain embodiments, stable viral producing cells permissive for baculovirus infection are engineered with at least one stable integrated copy of any of the elements necessary for AAV replication and vector production including, but not limited to, the entire AAV genome, Rep and Cap genes, Rep genes, Cap genes, each Rep protein as a separate transcription cassette, each VP protein as a separate transcription cassette, the AAP (assembly activation protein), or at least one of the baculovirus helper genes with native or non-native promoters.

In certain embodiments, the Baculovirus expression vectors (BEV) are based on the AcMNPV baculovirus or BmNPV baculovirus BmNPV.

In certain embodiments, the Baculovirus expression vectors (BEV) is a BEV in which the baculoviral v-cath gene has been deleted (“v-cath deleted BEV”) or mutated.

### Other

In certain embodiments expression hosts include, but are not limited to, bacterial species within the genera Escherichia, Bacillus, Pseudomonas, Salmonella.

In certain embodiments, a host cell which includes AAV rep and cap genes stably integrated within the cell's chromosomes, may be used for AAV particle production. In a non-limiting example, a host cell which has stably integrated in its chromosome at least two copies of an AAV rep gene and AAV cap gene may be used to produce the AAV particle according to the methods and constructs described in U.S. Pat. No. 7,238,526, the contents of which are incorporated herein by reference in their entirety.

In certain embodiments, the AAV particle can be produced in a host cell stably transformed with a molecule comprising the nucleic acid sequences which permit the regulated expression of a rare restriction enzyme in the host cell, as described in US20030092161 and EP1183380, the contents of which are herein incorporated by reference in their entirety.

In certain embodiments, production methods and cell lines to produce the AAV particle may include, but are not limited to those taught in PCT/US1996/010245. PCT/US1997/015716, PCT/US1997/015691, PCT/US1998/019479, PCT/US1998/019463, PCT/US2000/000415, PCT/US2000/040872, PCT/US2004/016614, PCT/US2007/010055, PCT/US1999/005870, PCT/US2000/004755, U.S. patent application Ser. No. 08/549,489, U.S. Ser. No. 08/462,014, U.S. Ser. No. 09/659,203, U.S. Ser. No. 10/246,447, U.S. Ser. No. 10/465,302, U.S. Pat. Nos. 6,281,010, 6,270,996, 6,261,551, 5,756,283 (Assigned to NIH), U.S. Pat. Nos. 6,428,988, 6,274,354, 6,943,019, 6,482,634, (Assigned to NIH: U.S. Pat. Nos. 7,238,526, 6,475,769), U.S. Pat. No. 6,365,394 (Assigned to NIH), U.S. Pat. Nos. 7,491,508, 7,291,498, 7,022,519, 6,485,966, 6,953,690, 6,258,595, EP2018421, EP1064393, EP1163354, EP835321, EP931158, EP950111, EP1015619, EP 1183380, EP2018421, EP1226264, EP1636370, EP 1163354, EP1064393, US20030032613, US20020102714, US20030073232, US20030040101 (Assigned to NIH), US20060003451, US20020090717, US20030092161, US20070231303, US20060211115, US20090275107, US2007004042, US20030119191, US20020019050, the contents of each of which are incorporated herein by reference in their entirety.

### Viral Production Systems

### Large-Scale Production

In certain embodiments, AAV particle production may be modified to increase the scale of production. Large scale viral production methods according to the present disclosure may include any of the processes or processing steps taught in U.S. Pat. Nos. 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088. WO1999014354. WO1999015685, WO1999047691, WO2000055342, WO2000075353 and WO2001023597, the contents of each of which are herein incorporated by reference by reference in their entirety.

Methods of increasing AAV particle production scale typically include increasing the number of viral production cells. In certain embodiments, viral production cells include adherent cells. To increase the scale of AAV particle production by adherent viral production cells, larger cell culture surfaces are required. In certain embodiments, large-scale production methods include the use of roller bottles to increase cell culture surfaces. Other cell culture substrates with increased surface areas are known in the art. Examples of additional adherent cell culture products with increased surface areas include, but are not limited to iCELLis (Pall Corp, Port Washington, N.Y.), CELLSTACK®, CELLCUBE® (Corning Corp., Corning, N.Y.) and NUNC™ CELL FACTORY™ (Thermo Scientific. Waltham, Mass.) In certain embodiments, large-scale adherent cell surfaces may include from about 1,000 cm² to about 100,000 cm².

In certain embodiments, large-scale viral production methods of the present disclosure may include the use of suspension cell cultures. Suspension cell culture can allow for significantly increased numbers of cells. Typically, the number of adherent cells that can be grown on about 10-50 cm² of surface area can be grown in about 1 cm³ volume in suspension.

In certain embodiments, large-scale cell cultures may include from about 10⁷ to about 10⁹ cells, from about 10⁸ to about 10¹⁰ cells, from about 10⁹ to about 10¹² cells or at least 10¹² cells. In certain embodiments, large-scale cultures may produce from about 10⁹ to about 10¹², from about 10¹⁰ to about 10¹³, from about 10¹¹ to about 10¹⁴, from about 10¹² to about 10¹⁵ or at least 10¹⁵ AAV particles.

Transfection of replication cells in large-scale culture formats may be carried out according to any methods known in the art. For large-scale adherent cell cultures, transfection methods may include, but are not limited to the use of inorganic compounds (e.g., calcium phosphate) organic compounds (e.g., polyethyleneimine (PEI)) or the use of non-chemical methods (e.g., electroporation). With cells grown in suspension, transfection methods may include, but are not limited to the use of inorganic compounds (e.g., calcium phosphate) organic compounds (e.g., polyethyleneimine (PEI)) or the use of non-chemical methods (e.g., electroporation). In certain embodiments, transfection of large-scale suspension cultures may be carried out according to the section entitled “Transfection Procedure” described in Feng, L. et al., 2008. Biotechnol Appl Biochem. 50:121-32, the contents of which are herein incorporated by reference in their entirety. According to such embodiments, PEI-DNA complexes may be formed for introduction of plasmids to be transfected. In certain embodiments, cells being transfected with PEI-DNA complexes may be ‘shocked’ prior to transfection. This includes lowering cell culture temperatures to 4° C. for a period of about 1 hour. In certain embodiments, cell cultures may be shocked for a period of from about 10 minutes to about 5 hours. In certain embodiments, cell cultures may be shocked at a temperature of from about 0° C. to about 20° C.

In certain embodiments, transfections may include one or more vectors for expression of an RNA effector molecule to reduce expression of nucleic acids from one or more payload construct. Such methods may enhance the production of AAV particles by reducing cellular resources wasted on expressing payload constructs. In certain embodiments, such methods may be carried according to those taught in US Publication No. US2014/0099666, the contents of which are herein incorporated by reference in their entirety.

### Bioreactors

In certain embodiments, cell culture bioreactors may be used for large scale production of AAV particles. In certain embodiments, bioreactors include stirred tank reactors. Such reactors generally include a vessel, typically cylindrical in shape, with a stirrer (e.g., impeller.) In certain embodiments, such bioreactor vessels may be placed within a water jacket to control vessel temperature and/or to minimize effects from ambient temperature changes.

Bioreactor vessel volume may range in size from about 500 ml to about 2 L, from about 1 L to about 5 L, from about 2.5 L to about 20 L, from about 10 L to about 50 L, from about 25 L to about 100 L, from about 75 L to about 500 L, from about 250 L to about 2.000 L, from about 1,000 L to about 10,000 L, from about 5,000 L to about 50,000 L or at least 50,000 L. Vessel bottoms may be rounded or flat. In certain embodiments, animal cell cultures may be maintained in bioreactors with rounded vessel bottoms.

In certain embodiments, bioreactor vessels may be warmed through the use of a thermocirculator. Thermocirculators pump heated water around water jackets. In certain embodiments, heated water may be pumped through pipes (e.g., coiled pipes) that are present within bioreactor vessels. In certain embodiments, warm air may be circulated around bioreactors, including, but not limited to air space directly above culture medium. Additionally, pH and C02 levels may be maintained to optimize cell viability.

In certain embodiments, bioreactors may comprise hollow-fiber reactors. Hollow-fiber bioreactors may support the culture of both anchorage dependent and anchorage independent cells. Further bioreactors may include, but are not limited to, packed-bed or fixed-bed bioreactors. Such bioreactors may comprise vessels with glass beads for adherent cell attachment. Further packed-bed reactors may comprise ceramic beads.

In certain embodiments, viral particles are produced through the use of a disposable bioreactor. In certain embodiments, bioreactors may include GE WAVE bioreactor, a GE Xcellerax Bioreactor, a Sartorius Biostat Bioreactor, a ThermoFisher Hyclone Bioreactor, or a Pall Allegro Bioreactor.

In certain embodiments, AAV particle production in cell bioreactor cultures may be carried out according to the methods or systems taught in U.S. Pat. Nos. 5,064,764, 6,194,191, 6,566,118, 8,137,948 or US Patent Application No. US2011/0229971, the contents of each of which are herein incorporated by reference in their entirety.

### Expansion of Viral Production Cell (VPC) Mixtures

In certain embodiments, an AAV particle or viral vector of the present disclosure may be produced in a viral production cell (VPC), such as an insect cell. Production cells can be sourced from a Cell Bank (CB) and are often stored in frozen cell banks.

In certain embodiments, a viral production cell from a Cell Bank is provided in frozen form. The vial of frozen cells is thawed, typically until ice crystal dissipate. In certain embodiments, the frozen cells are thawed at a temperature between 10-50° C., 15-40° C., 20-30° C., 25-50° C., 30-45° C., 35-40° C., or 37-39° C. In certain embodiments, the frozen viral production cells are thawed using a heated water bath.

In certain embodiments, a thawed CB cell mixture will have a cell density of 1.0×10⁴-1.0×10⁹ cells/mL. In certain embodiments, the thawed CB cell mixture has a cell density of 1.0×10⁴-2.5×10⁴ cells/mL, 2.5×10⁴-5.0×10⁴ cells/mL, 5.0×10⁴-7.5×10⁴ cells/mL, 7.5×10⁴-1.0×10⁵ cells/mL, 1.0×10⁵-2.5×10⁵ cells/mL, 2.5×10⁵-5.0×10⁵ cells/mL, 5.0×10⁵-7.5×10⁵ cells/mL, 7.5×10⁵-1.0×10⁶ cells/mL, 1.0×10⁶-2.5×10⁶ cells/mL, 2.5×10⁶-5.0×10⁶ cells/mL, 5.0×10⁶-7.5×10⁶ cells/mL, 7.5×10⁶-1.0×10⁷ cells/mL, 1.0×10⁷-2.5×10⁷ cells/mL, 2.5×10⁷-5.0×10⁷ cells/mL, 5.0×10⁷-7.5×10⁷ cells/mL, 7.5×10⁷-1.0×10⁸ cells/mL, 1.0×10⁸-2.5×10⁸ cells/mL, 2.5×10⁸-5.0×10⁸ cells/mL, 5.0×10⁸-7.5×10⁸ cells/mL, or 7.5×10⁸-1.0×10⁹ cells/mL.

In certain embodiments, the volume of the CB cell mixture is expanded. This process is commonly referred to as a Seed Train, Seed Expansion, or CB Cellular Expansion. Cellular/Seed expansion can include successive steps of seeding and expanding a cell mixture through multiple expansion steps using successively larger working volumes. In certain embodiments, cellular expansion can include one, two, three, four, five, six, seven, or more than seven expansion steps. In certain embodiments, the working volume in the cellular expansion can include one or more of the following working volumes or working volume ranges: 5 mL, 10 mL, 20 mL, 5-20 mL, 25 mL, 30 mL, 40 mL, 50 mL, 20-50 mL, 75 mL, 100 mL, 125 mL, 150 mL, 175 mL, 200 mL, 50-200 mL, 250 mL, 300 mL, 400 mL, 500 mL, 750 mL, 1000 mL, 250-1000 mL, 1250 mL, 1500 mL, 1750 mL, 2000 mL, 1000-2000 mL, 2250 mL, 2500 mL, 2750 mL, 3000 mL, 2000-3000 mL, 3500 mL, 4000 mL, 4500 mL, 5000 mL, 3000-5000 mL, 5.5 L, 6.0 L, 7.0 L, 8.0 L, 9.0 L, 10.0 L, and 5.0-10.0 L.

In certain embodiments, a volume of cells from a first expanded cell mixture can be used to seed a second, separate Seed Train/Seed Expansion (instead of using thawed CB cell mixture). This process is commonly referred to as rolling inoculum. In certain embodiments, rolling inoculum is used in a series of two or more (e.g., two, three, four or five) separate Seed Trains/Seed Expansions.

In certain embodiments, large-volume cellular expansion can include the use of a bioreactor, such as a GE WAVE bioreactor, a GE Xcellerax Bioreactor, a Sartorius Biostat Bioreactor, a ThermoFisher Hyclone Bioreactor, or a Pall Allegro Bioreactor.

In certain embodiments, the cell density within a working volume is expanded to a target output cell density. In certain embodiments, the output cell density of an expansion step is 1.0×10⁵-5.0×10⁵, 5.0×10⁵-1.0×10⁶, 1.0×10⁶-5.0×10⁶, 5.0×10⁶-1.0×10⁷, 1.0×10⁷-5.0×10⁷, 5.0×10⁷-1.0×10⁸, 5.0×10⁵, 6.0×10⁵, 7.0×10⁵, 8.0×10⁵, 9.0×10⁵, 1.0×10⁶, 2.0×10⁶, 3.0×10⁶, 4.0×10⁶, 5.0×10⁶, 6.0×10⁶, 7.0×10⁶, 8.0×10⁶, 9.0×10⁶, 1.0×10⁷, 2.0×10⁷, 3.0×10⁷, 4.0×10⁷, 5.0×10⁷, 6.0×10⁷, 7.0×10⁷, 8.0×10⁷, or 9.0×10⁷ cells/mL.

In certain embodiments, the output cell density of a working volume provides a seeding cell density for a larger, successive working volume. In certain embodiments, the seeding cell density of an expansion step is 1.0×10⁵-5.0×10⁵, 5.0×10⁵-1.0×10⁶, 1.0×10⁶-5.0×10⁶, 5.0×10⁶-1.0×10⁷, 1.0×10⁷-5.0×10⁷, 5.0×10⁷-1.0×10⁸, 5.0×10⁵, 6.0×10⁵, 7.0×10⁵, 8.0×10⁵, 9.0×10⁵, 1.0×10⁶, 2.0×10⁶, 3.0×10⁶, 4.0×10⁶, 5.0×10⁶, 6.0×10⁶, 7.0×10⁶, 8.0×10⁶, 9.0×10⁶, 1.0×10⁷, 2.0×10⁷, 3.0×10⁷, 4.0×10⁷, 5.0×10⁷, 6.0×10⁷, 7.0×10⁷, 8.0×10⁷, or 9.0×10⁷ cells/mL.

In certain embodiments, cellular expansion can last for 1-50 days. Each cellular expansion step or the total cellular expansion can last for 1-10 days, 1-5 days, 1-3 days, 2-3 days, 2-4 days, 2-5 days, 2-6 days, 3-4 days, 3-5 days, 3-6 days, 3-8 days, 4-5 days, 4-6 days, 4-8 days, 5-6 days, or 5-8 days. In certain embodiments, each cellular expansion step or the total cellular expansion can last for 1-100 generations, 1-1000 generations, 100-1000 generation, 100 generations or more, or 1000 generation or more.

In certain embodiments, infected or transfected production cells can be expanded in the same manner as CB cell mixtures, as set forth in the present disclosure.

### Infection of Viral Production Cells

In certain embodiments, AAV particles of the present disclosure are produced in a viral production cell (VPC), such as an insect cell, by infecting the VPC with a viral vector which includes an AAV expression construct and/or a viral vector which includes an AAV payload construct. In certain embodiments, the VPC is infected with an Expression BEV which includes an AAV expression construct and a Payload BEV which includes an AAV payload construct.

In certain embodiments, AAV particles are produced by infecting a VPC with a viral vector which includes both an AAV expression construct and an AAV payload construct. In certain embodiments, the VPC is infected with a single BEV which includes both an AAV expression construct and an AAV payload construct.

In certain embodiments, VPCs (such as insect cells) are infected using Infection BIICs in an infection process which includes the following steps: (i) A collection of VPCs are seeded into a Production Bioreactor; (ii) The seeded VPCs can optionally be expanded to a target working volume and cell density; (iii) Infection BIICs which include Expression BEVs and Infection BIICs which include Payload BEVs are injected into the Production Bioreactor, resulting in infected viral production cells; and (iv) incubation of the infected viral production cells to produce AAV particles within the viral production cells.

In certain embodiments, the VPC density at infection is 1.0×10⁵-2.5×10⁵, 2.5×10⁵-5.0×10⁵, 5.0×10⁵-7.5×10⁵, 7.5×10⁵

−1.0×10⁶, 1.0×10⁶-5.0×10⁶, 1.0×10⁶-2.0×10⁶, 1.5×10⁶-2.5×10⁶, 2.0×10⁶-3.0×10⁶, 2.5×10⁶-3.5×10⁶, 3.0×10⁶-4.0×10⁶, 3.5×10⁶-4.5×10⁶, 4.0×10⁶-5.0×10⁶, 4.5×10⁶-5.5×10⁶, 5.0×10⁶-1.0×10⁷, 5.0×10⁶-6.0×10⁶, 5.5×10⁶-6.5×10⁶, 6.0×10⁶-7.0×10⁶, 6.5×10⁶-7.5×10⁶, 7.0×10⁶-8.0×10⁶, 7.5×10⁶-8.5×10⁶, 8.0×10⁶-9.0×10⁶, 8.5×10⁶-9.5×10⁶, 9.0×10⁶-1.0×10⁷, 9.5×10⁶-1.5×10⁷, 1.0×10⁷-5.0×10⁷, or 5.0×10⁷-1.0×10⁸ cells/mL. In certain embodiments, the VPC density at infection is 5.0×10⁵, 6.0×10⁵, 7.0×10⁵, 8.0×10⁵, 9.0×10⁵, 1.0×10⁶, 1.5×10⁶, 2.0×10⁶, 2.5×10⁶, 3.0×10⁶, 3.5×10⁶, 4.0×10⁶, 4.5×10⁶, 5.0×10⁶, 5.5×10⁶, 6.0×10⁶, 6.5×10⁶, 7.0×10⁶, 7.5×10⁶, 8.0×10⁶, 8.5×10⁶, 9.0×10⁶, 9.5×10⁶, 1.0×10⁷, 1.5×10⁷, 2.0×10⁷, 2.5×10⁷, 3.0×10⁷, 4.0×10⁷, 5.0×10⁷, 6.0×10⁷, 7.0×10⁷, 8.0×10⁷, or 9.0×10⁷ cells/mL.

In certain embodiments, Infection BIICs are combined with the VPCs in target ratios of VPC-to-BIIC. In certain embodiments, the VPC-to-BIIC infection ratio (volume to volume) is 1.0×10³-5.0×10³, 5.0×10³-1.0×10⁴, 1.0×10⁴-5.0×10⁴, 5.0×10⁴-1.0×10⁵, 1.0×10⁵-5.0×10⁵, 5.0×10⁵-1.0×10⁶, 1.0×10³, 2.0×10³, 3.0×10³, 4.0×10³, 5.0×10³, 6.0×10³, 7.0×10³, 8.0×10³, 9.0×10³, 1.0×10⁴, 2.0×10⁴, 3.0×10⁴, 4.0×10⁴, 5.0×10⁴, 6.0×10⁴, 7.0×10⁴, 8.0×10⁴, or 9.0×10⁴, 1.0×10⁵, 2.0×10⁵, 3.0×10⁵, 4.0×10⁵, 5.0×10⁵, 6.0×10⁵, 7.0×10⁵, 8.0×10⁵, or 9.0×10⁵BIIC-per-VPC. In certain embodiments, the VPC-to-BIIC infection ratio (cell to cell) is 1.0×10³-5.0×10³, 5.0×10³-1.0×10⁴, 1.0×10⁴-5.0×10⁴, 5.0×10⁴-1.0×10⁵, 1.0×10⁵-5.0×10⁵, 5.0×10⁵-1.0×10⁶, 1.0×10³, 2.0×10³, 3.0×10³, 4.0×10³, 5.0×10³, 6.0×10³, 7.0×10³, 8.0×10³, 9.0×10³, 1.0×10⁴, 2.0×10⁴, 3.0×10⁴, 4.0×10⁴, 5.0×10⁴, 6.0×10⁴, 7.0×10⁴, 8.0×10⁴, or 9.0×10⁴, 1.0×10⁵, 2.0×10⁵, 3.0×10⁵, 4.0×10⁵, 5.0×10⁵, 6.0×10⁵, 7.0×10⁵, 8.0×10⁵, or 9.0×10⁵ BIIC-per-VPC.

In certain embodiments, Infection BIICs which include Expression BEVs and Infection BIICs which include Payload BEVs are combined with the VPCs in target BIIC-to-BIIC ratios. In certain embodiments, the ratio of Expression (Rep/Cap) BIICs to Payload BIICs is 10:1, 9:1, 8:1, 7:1, 6:1, 5:1, 4.5:1, 4:1.3.5:1, 3:1, 2.5:1, 2:1, 1.5:1, 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:9, 1:10, 3.5-4.5:1, 3-4:1, 2.5-3.5:1, 2-3:1, 1.5-2.5:1, 1-2:1, 1-1.5:1, 1:1-1.5, 1:1-2, 1:1.5-2.5, 1:2-3, 1:2.5-3.5, 1:3-4, 1:3.5-4.5, 1:4-5, 1:4.5-5.5, 1:5-6, 1:5.5-6.5, 1:6-7, or 1:6.5-7.5.

### Cell Lysis

Cells of the present disclosure, including, but not limited to viral production cells, may be subjected to cell lysis according to any methods known in the art. Cell lysis may be carried out to obtain one or more agents (e.g., viral particles) present within any cells of the disclosure. In certain embodiments, a bulk harvest of AAV particles and viral production cells is subjected to cell lysis according to the present disclosure.

In certain embodiments, cell lysis may be carried out according to any of the methods or systems presented in U.S. Pat. Nos. 7,326,555, 7,579,181, 7,048,920, 6,410,300, 6,436,394, 7,732,129, 7,510,875, 7,445,930, 6,726,907, 6,194,191, 7,125,706, 6,995,006, 6,676,935, 7,968,333, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference in their entirety.

Cell lysis methods and systems may be chemical or mechanical. Chemical cell lysis typically comprises contacting one or more cells with one or more lysis agent under chemical lysis conditions. Mechanical lysis typically comprises subjecting one or more cells to one or more lysis conditions and/or one or more lysis forces. Lysis can also be completed by allowing the cells to degrade after reaching ˜0% viability.

In certain embodiments, chemical lysis may be used to lyse cells. As used herein, the term “lysis agent” refers to any agent that may aid in the disruption of a cell. In certain embodiments, lysis agents are introduced in solutions, termed lysis solutions or lysis buffers. As used herein, the term “lysis solution” refers to a solution (typically aqueous) comprising one or more lysis agent. In addition to lysis agents, lysis solutions may include one or more buffering agents, solubilizing agents, surfactants, preservatives, cryoprotectants, enzymes, enzyme inhibitors and/or chelators. Lysis buffers are lysis solutions comprising one or more buffering agent. Additional components of lysis solutions may include one or more solubilizing agent. As used herein, the term “solubilizing agent” refers to a compound that enhances the solubility of one or more components of a solution and/or the solubility of one or more entities to which solutions are applied. In certain embodiments, solubilizing agents enhance protein solubility. In certain embodiments, solubilizing agents are selected based on their ability to enhance protein solubility while maintaining protein conformation and/or activity.

Exemplary lysis agents may include any of those described in U.S. Pat. Nos. 8,685,734, 7,901,921, 7,732,129, 7,223,585, 7,125,706, 8,236,495, 8,110,351, 7,419,956, 7,300,797, 6,699,706 and 6,143,567, the contents of each of which are herein incorporated by reference in their entirety. In certain embodiments, lysis agents may be selected from lysis salts, amphoteric agents, cationic agents, ionic detergents, and non-ionic detergents. Lysis salts may include, but are not limited to, sodium chloride (NaCl) and potassium chloride (KCl.) Further lysis salts may include any of those described in U.S. Pat. Nos. 8,614,101, 7,326,555, 7,579,181, 7,048,920, 6,410,300, 6,436,394, 7,732,129, 7,510,875, 7,445,930, 6,726,907, 6,194,191, 7,125,706, 6,995,006, 6,676,935 and 7,968,333, the contents of each of which are herein incorporated by reference in their entirety.

In certain embodiments, the cell lysate solution includes a stabilizing additive. In certain embodiments, the stabilizing additive can include trehalose, glycine betaine, mannitol, potassium citrate, CuCl2, proline, xylitol, NDSB 201, CTAB and K₂PO₄. In certain embodiments, the stabilizing additive can include amino acids such as arginine, or acidified amino acid mixtures such as arginine HCl. In certain embodiments, the stabilizing additive can include 0.1 M arginine or arginine HCl. In certain embodiments, the stabilizing additive can include 0.2 M arginine or arginine HCl. In certain embodiments, the stabilizing additive can include 0.25 M arginine or arginine HCl. In certain embodiments, the stabilizing additive can include 0.3 M arginine or arginine HCl. In certain embodiments, the stabilizing additive can include 0.4 M arginine or arginine HCl. In certain embodiments, the stabilizing additive can include 0.5 M arginine or arginine HCl. In certain embodiments, the stabilizing additive can include 0.6 M arginine or arginine HCl. In certain embodiments, the stabilizing additive can include 0.7 M arginine or arginine HCl. In certain embodiments, the stabilizing additive can include 0.8 M arginine or arginine HCl. In certain embodiments, the stabilizing additive can include 0.9 M arginine or arginine HCl. In certain embodiments, the stabilizing additive can include 1.0 M arginine or arginine HCl.

Concentrations of salts may be increased or decreased to obtain an effective concentration for the rupture of cell membranes. Amphoteric agents, as referred to herein, are compounds capable of reacting as an acid or a base. Amphoteric agents may include, but are not limited to lysophosphatidylcholine, 3-((3-Cholamidopropyl) dimethylammonium)-1-propanesulfonate (CHAPS). ZWITTERGENT® and the like. Cationic agents may include, but are not limited to, cetyltrimethylammonium bromide (C (16) TAB) and Benzalkonium chloride. Lysis agents comprising detergents may include ionic detergents or non-ionic detergents.

Detergents may function to break apart or dissolve cell structures including, but not limited to cell membranes, cell walls, lipids, carbohydrates, lipoproteins, and glycoproteins. Exemplary ionic detergents include any of those taught in U.S. Pat. Nos. 7,625,570 and 6,593,123 or US Publication No. US2014/0087361, the contents of each of which are herein incorporated by reference in their entirety. In certain embodiments, the lysis solution includes one or more ionic detergents. Example of ionic detergents for use in a lysis solution include, but are not limited to, sodium dodecyl sulfate (SDS), cholate and deoxycholate. In certain embodiments, ionic detergents may be included in lysis solutions as a solubilizing agent. In certain embodiments, the lysis solution includes one or more nonionic detergents. Non-ionic detergents for use in a lysis solution may include, but are not limited to, octylglucoside, digitonin, lubrol, C12E8, TWEEN®-20, TWEEN®-80, Triton X-100, Triton X-114, Brij-35, Brij-58, and Noniodet P-40. Non-ionic detergents are typically weaker lysis agents but may be included as solubilizing agents for solubilizing cellular and/or viral proteins. In certain embodiments, the lysis solution includes one or more zwitterionic detergents. Zwitterionic detergents for use in a lysis solution may include, but are not limited to: Lauryl dimethylamine N-oxide (LDAO); N,N-Dimethyl-N-dodecylglycine betaine (Empigen BB); 3-(N,N-Dimethylmyristylammonio) propanesulfonate (Zwittergent 3-10); n-Dodecyl-N,N-dimethyl-3-ammonio-1-propanesulfonate (Zwittergent 3-12); n-Tetradecyl-N,N-dimethyl-3-ammonio-1-propanesulfonate (Zwittergent 3-14); 3-(N,N-Dimethyl palmitylammonio) propanesulfonate (Zwittergent 3-16); 3-((3-cholamidopropyl) dimethylammonio)-1-propanesulfonate (CHAPS); and 3-([3-Cholamidopropyl] dimethylammonio)-2-hydroxy-1-propanesulfonate (CHAPSO).

In certain embodiments, the lysis solution includes Triton X-100, such as 0.5% w/v of Triton X-100. In certain embodiments, the lysis solution includes Lauryldimethylamine N-oxide (LDAO), such as 0.184% w/v (4×CMC) of LDAO. In certain embodiments, the lysis solution includes a seed oil surfactant such as Ecosurf SA-9. In certain embodiments, the lysis solution includes N,N-Dimethyl-N-dodecylglycine betaine (Empigen BB). In certain embodiments, the lysis solution includes a Zwittergent detergent, such as Zwittergent 3-12 (n-Dodecyl-N,N-dimethyl-3-ammonio-1-propanesulfonate), Zwittergent 3-14 (n-Tetradecyl-N,N-dimethyl-3-ammonio-1-propanesulfonate), or Zwittergent 3-16 (3-(N,N-Dimethyl palmitylammonio)propanesulfonate).

Further lysis agents may include enzymes and urea. In certain embodiments, one or more lysis agents may be combined in a lysis solution in order to enhance one or more of cell lysis and protein solubility. In certain embodiments, enzyme inhibitors may be included in lysis solutions in order to prevent proteolysis that may be triggered by cell membrane disruption.

In certain embodiments, the lysis solution includes between 0.1-1.0% w/v, between 0.2-0.8% w/v, between 0.3-0.7% w/v, between 0.4-0.6% w/v, or about 0.5% w/v of a cell lysis agent (e.g., detergent). In certain embodiments, the lysis solution includes between 0.3-0.35% w/v, between 0.35-0.4% w/v, between 0.4-0.45% w/v, between 0.45-0.5% w/v, between 0.5-0.55% w/v, between 0.55-0.6% w/v, between 0.6-0.65% w/v, or between 0.65-0.7% w/v of a cell lysis agent (e.g., detergent).

In certain embodiments, cell lysates generated from adherent cell cultures may be treated with one more nuclease, such as Benzonase nuclease (Grade 1, 99% pure) or c-LEcta Denarase nuclease (formerly Sartorius Denarase). In certain embodiments, nuclease is added to lower the viscosity of the lysates caused by liberated DNA.

In certain embodiments, chemical lysis uses a single chemical lysis mixture. In certain embodiments, chemical lysis uses several lysis agents added in series to provide a final chemical lysis mixture.

In certain embodiments, a chemical lysis mixture includes an acidified amino acid mixture (such as arginine HCl), a non-ionic detergent (such as Triton X-100), and a nuclease (such as Benzonase nuclease). In certain embodiments, the chemical lysis mixture can include an acid or base to provide a target lysis pH.

In certain embodiments, chemical lysis is conducted under chemical lysis conditions. As used herein, the term “chemical lysis conditions” refers to any combination of environmental conditions (e.g., temperature, pressure, pH, etc) in which targets cells can be lysed by a lysis agent.

In certain embodiments, the lysis pH is between 3.0-3.5, 3.5-4.0, 4.0-4.5, 4.5-5.0, 5.0-5.5, 5.5-6.0, 6.0-6.5, 6.5-7.0, 7.0-7.5, or 7.5-8.0.

In certain embodiments, the lysis temperature is between 15-35° C., between 20-30° C., between 25-39° C., between 20-21° C. between 20-22° C., between 21-22° C., between 21-23° C., between 22-23° C., between 22-24° C., between 23-24° C., between 23-25° C., between 24-25° C. between 24-26° C., between 25-26° C., between 25-27° C., between 26-27° C., between 26-28° C., between 27-28° C., between 27-29° C., between 28-29° C. between 28-30° C., between 29-30° C., between 29-31° C., between 30-31° C., between 30-32° C., between 31-32° C., or between 31-33° C.,

In certain embodiments, mechanical cell lysis is carried out. Mechanical cell lysis methods may include the use of one or more lysis condition and/or one or more lysis force. As used herein, the term “lysis condition” refers to a state or circumstance that promotes cellular disruption. Lysis conditions may comprise certain temperatures, pressures, osmotic purity, salinity, and the like. In certain embodiments, lysis conditions comprise increased or decreased temperatures. According to certain embodiments, lysis conditions comprise changes in temperature to promote cellular disruption. Cell lysis carried out according to such embodiments may include freeze-thaw lysis. As used herein, the term “freeze-thaw lysis” refers to cellular lysis in which a cell solution is subjected to one or more freeze-thaw cycle. According to freeze-thaw lysis methods, cells in solution are frozen to induce a mechanical disruption of cellular membranes caused by the formation and expansion of ice crystals. Cell solutions used according freeze-thaw lysis methods, may further comprise one or more lysis agents, solubilizing agents, buffering agents, cryoprotectants, surfactants, preservatives, enzymes, enzyme inhibitors and/or chelators. Once cell solutions subjected to freezing are thawed, such components may enhance the recovery of desired cellular products. In certain embodiments, one or more cryoprotectants are included in cell solutions undergoing freeze-thaw lysis. As used herein, the term “cryoprotectant” refers to an agent used to protect one or more substance from damage due to freezing. Cryoprotectants may include any of those taught in US Publication No. US2013/0323302 or U.S. Pat. Nos. 6,503,888, 6,180,613, 7,888,096, 7,091,030, the contents of each of which are herein incorporated by reference in their entirety. In certain embodiments, cryoprotectants may include, but are not limited to dimethyl sulfoxide, 1,2-propanediol, 2,3-butanediol, formamide, glycerol, ethylene glycol, 1,3-propanediol and n-dimethyl formamide, polyvinylpyrrolidone, hydroxyethyl starch, agarose, dextrans, inositol, glucose, hydroxyethylstarch, lactose, sorbitol, methyl glucose, sucrose, and urea. In certain embodiments, freeze-thaw lysis may be carried out according to any of the methods described in U.S. Pat. No. 7,704,721, the contents of which are herein incorporated by reference in their entirety.

As used herein, the term “lysis force” refers to a physical activity used to disrupt a cell. Lysis forces may include, but are not limited to mechanical forces, sonic forces, gravitational forces, optical forces, electrical forces and the like. Cell lysis carried out by mechanical force is referred to herein as “mechanical lysis.” Mechanical forces that may be used according to mechanical lysis may include high shear fluid forces. According to such methods of mechanical lysis, a microfluidizer may be used. Microfluidizers typically comprise an inlet reservoir where cell solutions may be applied. Cell solutions may then be pumped into an interaction chamber via a pump (e.g., high-pressure pump) at high speed and/or pressure to produce shear fluid forces. Resulting lysates may then be collected in one or more output reservoir. Pump speed and/or pressure may be adjusted to modulate cell lysis and enhance recovery of products (e.g., viral particles.) Other mechanical lysis methods may include physical disruption of cells by scraping.

Cell lysis methods may be selected based on the cell culture format of cells to be lysed. For example, with adherent cell cultures, some chemical and mechanical lysis methods may be used. Such mechanical lysis methods may include freeze-thaw lysis or scraping. In another example, chemical lysis of adherent cell cultures may be carried out through incubation with lysis solutions comprising surfactant, such as Triton-X-100.

In certain embodiments, a method for harvesting AAV particles without lysis may be used for efficient and scalable AAV particle production. In a non-limiting example, AAV particles may be produced by culturing an AAV particle lacking a heparin binding site, thereby allowing the AAV particle to pass into the supernatant, in a cell culture, collecting supernatant from the culture; and isolating the AAV particle from the supernatant, as described in US Patent Application 20090275107, the contents of which are incorporated herein by reference in their entirety.

### Clarification and Purification: General

Cell lysates comprising viral particles may be subjected to clarification and purification. Clarification generally refers to the initial steps taken in the purification of viral particles from cell lysates and serves to prepare lysates for further purification by removing larger, insoluble debris from a bulk lysis harvest. Viral production can include clarification steps at any point in the viral production process. Clarification steps may include, but are not limited to, centrifugation and filtration. During clarification, centrifugation may be carried out at low speeds to remove larger debris only. Similarly, filtration may be carried out using filters with larger pore sizes so that only larger debris is removed.

Purification generally refers to the final steps taken in the purification and concentration of viral particles from cell lysates by removing smaller debris from a clarified lysis harvest in preparing a final Pooled Drug Substance. Viral production can include purification steps at any point in the viral production process. Purification steps may include, but are not limited to, filtration and chromatography. Filtration may be carried out using filters with smaller pore sizes to remove smaller debris from the product or with larger pore sizes to retain larger debris from the product. Filtration may be used to alter the concentration and/or contents of a viral production pool or stream. Chromatography may be carried out to selectively separate target particles from a pool of impurities.

Large scale production of high-concentration AAV formulations is complicated by the tendency for high concentrations of AAV particles to aggregate or agglomerate. Small scale clarification and concentration systems, such as dialysis cassettes or spin centrifugation, are generally not sufficiently scalable for large-scale production. The present disclosure provides embodiments of a clarification, purification, and concentration system for processing large volumes of high-concentration AAV production formulations. In certain embodiments, the large-volume clarification system comprises one or more of the following processing steps: Depth Filtration, Microfiltration (e.g., 0.2 μm Filtration), Affinity Chromatography, Ion Exchange Chromatography such as anion exchange chromatography (AEX) or cation exchange chromatography (CEX), a tangential flow filtration system (TFF), Nanofiltration (e.g., Virus Retentive Filtration (VRF)), Final Filtration (FF), and Fill Filtration.

Objectives of viral clarification and purification include high throughput processing of cell lysates and to optimize ultimate viral recovery. Advantages of including clarification and purification steps of the present disclosure include scalability for processing of larger volumes of lysate. In certain embodiments, clarification and purification may be carried out according to any of the methods or systems presented in U.S. Pat. Nos. 8,524,446, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498, 7,491,508, US Publication Nos. US2013/0045186, US2011/0263027, US2011/0151434, US2003/0138772, and International Publication Nos. WO2002012455, WO1996039530, WO1998010088, WO1999014354, WO1999015685, WO1999047691, WO2000055342, WO2000075353 and WO2001023597, the contents of each of which are herein incorporated by reference in their entirety.

In certain embodiments, the compositions comprising at least one AAV particle may be isolated or purified using the methods or systems described in U.S. Pat. Nos. 6,146,874, 6,660,514, 8,283,151 or U.S. Pat. No. 8,524,446, the contents of which are herein incorporated by reference in their entirety.

### Clarification and Purification: Centrifugation

According to certain embodiments, cell lysates may be clarified by one or more centrifugation steps. Centrifugation may be used to pellet insoluble particles in the lysate. During clarification, centrifugation strength (which can be expressed in terms of gravitational units (g), which represents multiples of standard gravitational force) may be lower than in subsequent purification steps. In certain embodiments, centrifugation may be carried out on cell lysates at a gravitation force from about 200 g to about 800 g, from about 500 g to about 1500 g, from about 1000 g to about 5000 g, from about 1200 g to about 10000 g or from about 8000 g to about 15000 g. In certain embodiments, cell lysate centrifugation is carried out at 8000 g for 15 minutes. In certain embodiments, density gradient centrifugation may be carried out in order to partition particulates in the cell lysate by sedimentation rate. Gradients used according to methods or systems of the present disclosure may include, but are not limited to, cesium chloride gradients and iodixanol step gradients. In certain embodiments, centrifugation uses a decanter centrifuge system. In certain embodiments, centrifugation uses a disc-stack centrifuge system. In certain embodiments, centrifugation includes ultracentrifugation, such two-cycle CsCl gradient ultracentrifugation or iodixanol discontinuous density gradient ultracentrifugation.

### Clarification and Purification: Filtration

In certain embodiments, one or more microfiltration, nanofiltration and/or ultrafiltration steps may be used during clarification, purification and/or sterilization. The one or more microfiltration, nanofiltration or ultrafiltration steps can include the use of a filtration system such as EMD Millipore Express SHC XL IO 0.5/0.2 μm filter, EMD Millipore Express SHCXL6000 0.5/0.2 μm filter, EMD Millipore Express SHCXL150 filter, EMD Millipore Millipak Gamma Gold 0.22 μm filter (dual-in-line sterilizing grade filters), a Pall Supor EKV, 0.2 μm sterilizing-grade filter, Asahi Planova 35N, Asahi Planova 20N, Asahi Planova 75N, Asahi Planova BioEx, Millipore Viresolve NFR or a Sartorius Sartopore 2XLG, 0.8/0.2 μm.

In certain embodiments, one or more microfiltration steps may be used during clarification, purification and/or sterilization. Microfiltration utilizes microfiltration membranes with pore sizes typically between 0.1 μm and 10 μm. Microfiltration is generally used for general clarification, sterilization, and removal of microparticulates. In certain embodiments, microfiltration is used to remove aggregated clumps of viral particles. In certain embodiments, a production process or system of the present disclosure includes at least one microfiltration step. The one or more microfiltration steps can include a Depth Filtration step with a Depth Filtration system, such as EMD Millipore Millistak⁺ POD filter (D0HC media series), Millipore MC0SP23CL3 filter (C0SP media series), or Sartorius Sartopore filter series. Microfiltration systems of the present disclosure can be pre-rinsed, packed, equilibrated, flushed, processed, eluted, washed, or cleaned with formulations known to those in the art, including AAV pharmaceutical, processing and storage formulations of the present disclosure.

In certain embodiments, one or more ultrafiltration steps may be used during clarification and purification. The ultrafiltration steps can be used for concentrating, formulating, desalting, or dehydrating either processing and/or formulation solutions of the present disclosure. Ultrafiltration utilizes ultrafiltration membranes, with pore sizes typically between 0.001 and 0.1 μm. Ultrafiltration membranes can also be defined by their molecular weight cutoff (MWCO) and can have a range from 1 kD to 500 kD. Ultrafiltration is generally used for concentrating and formulating dissolved biomolecules such as proteins, peptides, plasmids, viral particles, nucleic acids, and carbohydrates. Ultrafiltration systems of the present disclosure can be pre-rinsed, packed, equilibrated, flushed, processed, eluted, washed or cleaned with formulations known to those in the art, including AAV pharmaceutical, processing and storage formulations of the present disclosure.

In certain embodiments, one or more nanofiltration steps may be used during clarification and purification. Nanofiltration utilizes nanofiltration membranes, with pore sizes typically less than 100 nm. Nanofiltration is generally used for removal of unwanted endogenous viral impurities (e.g., baculovirus). In certain embodiments, nanofiltration can include viral removal filtration (VRF). VRF filters can have a filtration size typically between 15 nm and 100 nm. Examples of VRF filters include (but are not limited to): Planova 15N, Planova 20N, and Planova 35N (Asahi-Kasei Corp, Tokyo, Japan); and Viresolve NFP and Viresolve NFR (Millipore Corp, Billerica, Mass., USA). Nanofiltration systems of the present disclosure can be pre-rinsed, packed, equilibrated, flushed, processed, eluted, washed, or cleaned with formulations known to those in the art, including AAV pharmaceutical, processing and storage formulations of the present disclosure. In certain embodiments, nanofiltration is used to remove aggregated clumps of viral particles.

In certain embodiments, one or more tangential flow filtration (TFF) (also known as cross-flow filtration) steps may be used during clarification and purification. Tangential flow filtration is a form of membrane filtration in which a feed stream (which includes the target agent/particle to be clarified and concentrated) flows from a feed tank into a filtration module or cartridge. Within the TFF filtration module, the feed stream passes parallel to a membrane surface, such that one portion of the stream passes through the membrane (permeate/filtrate) while the remainder of the stream (retentate) is recirculated back through the filtration system and into the feed tank.

In certain embodiments, the TFF filtration module can be a flat plate module (stacked planar cassette), a spiral wound module (spiral-wound membrane layers), or a hollow fiber module (bundle of membrane tubes). Examples of TFF systems for use in the present disclosure include, but are not limited to: Spectrum mPES Hollow Fiber TFF system (0.5 mm fiber ID, 100 kDA MWCO) or Millipore Ultracel PLCTK system with Pellicon-3 cassette (0.57 m², 30 kDA MWCO).

New buffer materials can be added to the TFF feed tank as the feed stream is circulated through the TFF filtration system. In certain embodiments, buffer materials can be fully replenished as the flow stream circulates through the TFF filtration system. In this embodiment, buffer material is added to the stream in equal amounts to the buffer material lost in the permeate, resulting in a constant concentration. In certain embodiments, buffer materials can be reduced as the flow stream circulates through the filtration system. In this embodiment, a reduced amount of buffer material is added to the stream relative to the buffer material lost in the permeate, resulting in an increased concentration. In certain embodiments, buffer materials can be replaced as the flow stream circulates through the filtration system. In this embodiment, the buffer added to stream is different from buffer materials lost in the permeate, resulting in an eventual replacement of buffer material in the stream. TFF systems of the present disclosure can be pre-rinsed, packed, equilibrated, flushed, processed, eluted, washed, or cleaned with formulations known to those in the art, including AAV pharmaceutical, processing and storage formulations of the present disclosure.

In certain embodiments, a TFF load pool can be spiked with an excipient or diluent prior to filtration. In certain embodiments, a TFF load pool is spiked with a high-salt mixture (such as sodium chloride or potassium chloride) prior to filtration. In certain embodiments, a TFF load pool is spiked with a high-sugar mixture (such as 50% w/v sucrose) prior to filtration.

The effectiveness of TFF processing can depend on several factors, including (but not limited to): shear stress from flow design, cross-flow rate, filtrate flow control, transmembrane pressure (TMP), membrane conditioning, membrane composition (e.g., hollow fiber construction) and design (e.g. surface area), system flow design, reservoir design, and mixing strategy. In certain embodiment, the filtration membrane can be exposed to pre-TFF membrane conditioning.

In certain embodiments, TFF processing can include one or more microfiltration stages. In certain embodiments, TFF processing can include one or more ultrafiltration stages. In certain embodiments, TFF processing can include one or more nanofiltration stages.

In certain embodiments, TFF processing can include one or more concentration stages, such as an ultrafiltration (UF) or microfiltration (MF) concentration stage. In the concentration stage, a reduced amount of buffer material is replaced as the stream circulates through the filtration system (relative to the amount of buffer material lost as permeate). The failure to completely replace all of the buffer material lost in the permeate results in an increased concentration of viral particles within the filtration stream. In certain embodiments, an increased amount of buffer material is replaced as the stream circulates through the filtration system. The incorporation of excess buffer material relative to the amount of buffer material lost in the permeate results in a decreased concentration of viral particles within the filtration stream.

In certain embodiments, TFF processing can include one or more diafiltration (DF) stages. The diafiltration stage includes replacement of a first buffer material (such as a high salt material) within a second buffer material (such a low-salt or zero-salt material). In this embodiment, a second buffer is added to flow stream which is different from a first buffer material lost in the permeate, resulting in an eventual replacement of buffer material in the stream.

In certain embodiments, TFF processing can include multiple stages in series. In certain embodiments, a TFF processing process can include an ultrafiltration (UF) concentration stage followed by a diafiltration stage (DF). In certain embodiments, a TFF processing can include a diafiltration stage followed by an ultrafiltration concentration stage. In certain embodiments, a TFF processing can include a first diafiltration stage, followed by an ultrafiltration concentration stage, followed by a second diafiltration stage. In certain embodiments, a TFF processing can include a first diafiltration stage which incorporates a high-salt-low-sugar buffer material into the flow stream, followed by an ultrafiltration/concentration stage which results in a high concentration of the viral material in the flow stream, followed by a second diafiltration stage which incorporates a low-salt-high-sugar or zero-salt-high-sugar buffer material into the flow stream. In certain embodiments, the salt can be sodium chloride, sodium phosphate, potassium chloride, potassium phosphate, or a combination thereof. In certain embodiments, the sugar can be sucrose, such as a 5% w/v sucrose mixture or a 7% w/v sucrose mixture.

In certain embodiments, TFF processing can include multiple stages which occur contemporaneously. As a non-limiting example, a TFF clarification process can include an ultrafiltration stage which occurs contemporaneously with a concentration stage.

Methods of cell lysate clarification and purification by filtration are well understood in the art and may be carried out according to a variety of available methods including, but not limited to passive filtration and flow filtration. Filters used may comprise a variety of materials and pore sizes. For example, cell lysate filters may comprise pore sizes of from about 1 μM to about 5 μM, from about 0.5 μM to about 2 μM, from about 0.1 μM to about 1 μM, from about 0.05 μM to about 0.05 μM and from about 0.001 μM to about 0.1 μM. Exemplary pore sizes for cell lysate filters may include, but are not limited to, 2.0, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1, 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, 0.22, 0.21, 0.20, 0.19, 0.18, 0.17, 0.16, 0.15, 0.14, 0.13, 0.12, 0.11, 0.1, 0.09, 0.08, 0.07, 0.06, 0.05, 0.04, 0.03, 0.02, 0.01, 0.02, 0.019, 0.018, 0.017, 0.016, 0.015, 0.014, 0.013, 0.012, 0.011, 0.01, 0.009, 0.008, 0.007, 0.006, 0.005, 0.004, 0.003, 0.002, 0.001 and 0.001 μM. In certain embodiments, clarification may comprise filtration through a filter with 2.0 μM pore size to remove large debris, followed by passage through a filter with 0.45 μM pore size to remove intact cells.

Filter materials may be composed of a variety of materials. Such materials may include, but are not limited to, polymeric materials and metal materials (e.g., sintered metal and pored aluminum.) Exemplary materials may include, but are not limited to nylon, cellulose materials (e.g., cellulose acetate), polyvinylidene fluoride (PVDF), polyethersulfone, polyamide, polysulfone, polypropylene, and polyethylene terephthalate. In certain embodiments, filters useful for clarification of cell lysates may include, but are not limited to ULTIPLEAT PROFILE™ filters (Pall Corporation. Port Washington, N.Y.), SUPOR™ membrane filters (Pall Corporation, Port Washington, N.Y.).

In certain embodiments, flow filtration may be carried out to increase filtration speed and/or effectiveness. In certain embodiments, flow filtration may comprise vacuum filtration. According to such methods, a vacuum is created on the side of the filter opposite that of cell lysate to be filtered. In certain embodiments, cell lysates may be passed through filters by centrifugal forces. In certain embodiments, a pump is used to force cell lysate through clarification filters. Flow rate of cell lysate through one or more filters may be modulated by adjusting one of channel size and/or fluid pressure.

### Clarification and Purification: Chromatography

In certain embodiments, AAV particles in a formulation may be clarified and purified from cell lysates through one or more chromatography steps using one or more different methods of chromatography. Chromatography refers to any number of methods known in the art for selectively separating out one or more elements from a mixture. Such methods may include, but are not limited to, ion exchange chromatography (e.g. cation exchange chromatography and anion exchange chromatography), affinity chromatography (e.g. immunoaffinity chromatography, metal affinity chromatography, pseudo affinity chromatography such as Blue Sepharose resins), hydrophobic interaction chromatography, size-exclusion chromatography, and multimodal chromatography (chromatographic methods that utilize more than one form of interaction between the stationary phase and analytes). In certain embodiments, methods or systems of viral chromatography may include any of those taught in U.S. Pat. Nos. 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference in their entirety.

Chromatography systems of the present disclosure can be pre-rinsed, packed, equilibrated, flushed, processed, eluted, washed, or cleaned with formulations known to those in the art, including AAV pharmaceutical, processing and storage formulations of the present disclosure.

In certain embodiments, one or more ion exchange (IEX) chromatography steps may be used to isolate viral particles. The ion exchange step can include anion exchange (AEX) chromatography, cation exchange (CEX) chromatography, or a combination thereof. In certain embodiments, ion exchange chromatography is used in a bind/elute mode. Bind/elute IEX can be used by binding viral particles to a stationary phase based on charge-charge interactions between capsid proteins (or other charged components) of the viral particles and charged sites present on the stationary phase. This process can include the use of a column through which viral preparations (e.g. clarified lysates) are passed. After application of viral preparations to the charged stationary phase (e.g., column), bound viral particles may then be eluted from the stationary phase by applying an elution solution to disrupt the charge-charge interactions. Elution solutions may be optimized by adjusting salt concentration and/or pH to enhance recovery of bound viral particles. Depending on the charge of viral capsids being isolated, cation or anion exchange chromatography methods may be selected. In certain embodiments, ion exchange chromatography is used in a flow-through mode. Flow-through IEX can be used by binding non-viral impurities or unwanted viral particles to a stationary phase (based on charge-charge interactions) and allowing the target viral particles in the viral preparation to “flow through” the IEX system into a collection pool.

Methods or systems of ion exchange chromatography may include, but are not limited to any of those taught in U.S. Pat. Nos. 7,419,817, 6,143,548, 7,094,604, 6,593,123, 7,015,026 and 8,137,948, the contents of each of which are herein incorporated by reference in their entirety. In certain embodiments, the IEX process uses an AEX chromatography system such as a Sartorius Sartobind Q membrane, a Millipore Fractogel TMAE HiCap(m) Flow-Through membrane, a GE Q Sepharose HP membrane, Poros XQ or Poros HQ. In certain embodiments, the IEX process uses a CEX system such as a Poros XS membrane. In certain embodiments, the AEX system includes a stationary phase which includes a trimethylammoniumethyl (TMAE) functional group.

In certain embodiments, one or more affinity chromatography steps, such as immunoaffinity chromatography, may be used to isolate viral particles. Immunoaffinity chromatography is a form of chromatography that utilizes one or more immune compounds (e.g. antibodies or antibody-related structures) to retain viral particles. Immune compounds may bind specifically to one or more structures on viral particle surfaces, including, but not limited to one or more viral coat protein. In certain embodiments, immune compounds may be specific for a particular viral variant. In certain embodiments, immune compounds may bind to multiple viral variants. In certain embodiments, immune compounds may include recombinant single-chain antibodies. Such recombinant single chain antibodies may include those described in Smith, R. H. et al., 2009. Mol. Ther. 17(11):1888-96, the contents of which are herein incorporated by reference in their entirety. Such immune compounds are capable of binding to several AAV capsid variants, including, but not limited to AAV1, AAV2, AAV6 and AAV8 or any of those taught herein. In certain embodiments, the AFC process uses a GE AVB Sepharose HP column resin, Poros CaptureSelect AAV8 resins (ThermoFisher), Poros CaptureSelect AAV9 resins (ThermoFisher) and Poros CaptureSelect AAVX resins (ThermoFisher).

In certain embodiments, one or more size-exclusion chromatography (SEC) steps may be used to isolate viral particles. SEC may comprise the use of a gel to separate particles according to size. In viral particle purification, SEC filtration is sometimes referred to as “polishing.” In certain embodiments, SEC may be carried out to generate a final product that is near-homogenous. Such final products may in certain embodiments be used in pre-clinical studies and/or clinical studies (Kotin, R. M. 2011. Human Molecular Genetics. 20(1):R2-R6, the contents of which are herein incorporated by reference in their entirety.) In certain embodiments, SEC may be carried out according to any of the methods taught in U.S. Pat. Nos. 6,143,548, 7,015,026, 8,476,418, 6,410,300, 8,476,418, 7,419,817, 7,094,604, 6,593,123, and 8,137,948, the contents of each of which are herein incorporated by reference in their entirety.

### III. Compositions and Formulations

### General

Gene therapy drug products (such as rAAV particles) are challenging to incorporate into composition and formulations due to their limited stability in the liquid state and a high propensity for large-scale aggregation at low concentrations. Gene therapy drug products are often delivered directly to treatment areas (including CNS tissue); which requires that excipients and formulation parameters be compatible with tissue function, microenvironment, and volume restrictions.

According to the present disclosure, AAV particles may be prepared as, or included in, pharmaceutical compositions. It will be understood that such compositions necessarily include one or more active ingredients and, most often, one or more pharmaceutically acceptable excipients.

Relative amounts of the active ingredient (e.g. AAV particle), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may include between 0.1% and 99% (w/w) of the active ingredient. By way of example, the composition may include between 0.1% and 100%. e.g., between 0.5 and 50%, between 1-30%, between 5-80%, or at least 80% (w/w) active ingredient.

In certain embodiments, the AAV particle pharmaceutical compositions described herein may include at least one payload of the present disclosure. As a non-limiting example, the pharmaceutical compositions may contain an AAV particle with 1, 2, 3, 4 or 5 payloads.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g., non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, rats, birds, including commercially relevant birds such as poultry, chickens, ducks, geese, and/or turkeys.

In certain embodiments, compositions are administered to humans, human patients, or subjects.

Formulations of the present disclosure can include, without limitation, saline, liposomes, lipid nanoparticles, polymers, peptides, proteins, cells transfected with AAV particles (e.g., for transfer or transplantation into a subject) and combinations thereof.

Formulations of the pharmaceutical compositions described herein may be prepared by any method known or hereafter developed in the art of pharmacology. As used herein the term “pharmaceutical composition” refers to compositions comprising at least one active ingredient and optionally one or more pharmaceutically acceptable excipients.

In general, such preparatory methods include the step of associating the active ingredient with an excipient and/or one or more other accessory ingredients. As used herein, the phrase “active ingredient” generally refers either to an AAV particle carrying a payload region encoding the polynucleotide or polypeptides of the present disclosure or to the end product encoded by a viral genome of an AAV particle as described herein.

In some embodiments, the formulations may comprise at least one inactive ingredient. As used herein, the term “inactive ingredient” refers to one or more inactive agents included in formulations. In some embodiments, all, none or some of the inactive ingredients which may be used in the formulations of the present disclosure may be approved by the US Food and Drug Administration (FDA).

Formulations of the AAV particles and pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

In certain embodiments, formulations of the present disclosure are aqueous formulations (i.e., formulations which include water). In certain embodiments, formulations of the present disclosure include water, sanitized water, or Water-for-injection (WFI).

In certain embodiments, the AAV particles of the present disclosure may be formulated in PBS with 0.001%-0.1% (w/v) of Poloxamer 188 (e.g., Pluronic F-68) at a pH of about 7.0.

In certain embodiments, the AAV formulations described herein may contain sufficient AAV particles for expression of at least one expressed functional payload. As a non-limiting example, the AAV particles may contain viral genomes encoding 1, 2, 3, 4 or 5 functional payloads.

According to the present disclosure AAV particles may be formulated for CNS delivery. Agents that cross the brain blood barrier may be used. For example, some cell penetrating peptides that can target molecules to the brain blood barrier endothelium may be used for formulation (e.g., Mathupala, Expert Opin Ther Pat., 2009, 19, 137-140; the content of which is incorporated herein by reference in its entirety).

In certain embodiments, the AAV formulations described herein may include a buffering system which includes phosphate, Tris, and/or Histidine. The buffering agents of phosphate, Tris, and/or Histidine may be independently used in the formulation in a range of 2-12 mM.

Formulations of the present disclosure can be used in any step of producing, processing, preparing, storing, expanding, or administering AAV particles and viral vectors of the present disclosure. In certain embodiments, pharmaceutical formulations and components can be use in AAV production, AAV processing, AAV clarification, AAV purification, and AAV finishing systems of the present disclosure, all of which can be pre-rinsed, packed, equilibrated, flushed, processed, eluted, washed, or cleaned with formulations known to those in the art, including AAV pharmaceutical, processing and storage formulations of the present disclosure.

### Excipients and Diluents

The AAV particles of the present disclosure can be formulated into a pharmaceutical composition which includes one or more excipients or diluents to (1) increase stability; (2) increase cell transfection or transduction; (3) permit the sustained or delayed release of the payload; (4) alter the biodistribution (e.g., target the viral particle to specific tissues or cell types); (5) increase the translation of encoded protein; (6) alter the release profile of encoded protein and/or (7) allow for regulatable expression of the payload of the present disclosure.

Relative amounts of the active ingredient (e.g., AAV particle), the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. In certain embodiments, the composition may comprise between 0.001% and 99% (w/w) of the active ingredient. By way of example, the composition may comprise between 0.001% and 100%, e.g., between 0.5 and 50%, between 1-30%, between 5-80%, or at least 80% (w/w) active ingredient. In certain embodiments, the composition may comprise between 0.001% and 99% (w/w) of the excipients and diluents. By way of example, the composition may comprise between 0.001% and 100%, e.g., between 0.5 and 50%, between 1-30%, between 5-80%, or at least 80% (w/w) excipients and diluents.

In certain embodiments, a pharmaceutically acceptable excipient may be at least 95%, at least 96%/6, at least 97%, at least 98%, at least 99%, or 100% pure. In certain embodiments, an excipient is approved for use for humans and for veterinary use. In certain embodiments, an excipient may be approved by United States Food and Drug Administration. In certain embodiments, an excipient may be of pharmaceutical grade. In certain embodiments, an excipient may meet the standards of the United States Pharmacopoeia (USP), the European Pharmacopoeia (EP), the British Pharmacopoeia, and/or the International Pharmacopoeia.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition. A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary excipients and diluents which can be included in formulations of the present disclosure include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary excipients and diluents which can be included in formulations of the present disclosure include, but are not limited to, 1,2,6-Hexanetriol; 1,2-Dimyristoyl-Sn-Glycero-3-(Phospho-S-(1-Glycerol)); 1,2-Dimyristoyl-Sn-Glycero-3-Phosphocholine; 1,2-Dioleoyl-Sn-Glycero-3-Phosphocholine; 1,2-Dipalmitoyl-Sn-Glycero-3-(Phospho-Rac-(1-Glycerol)); 1,2-Distearoyl-Sn-Glycero-3-(Phospho-Rac-(1-Glycerol)); 1,2-Distearoyl-Sn-Glycero-3-Phosphocholine; 1-O-Tolylbiguanide; 2-Ethyl-1,6-Hexanediol; Acetic Acid; Acetic Acid, Glacial; Acetic Anhydride; Acetone; Acetone Sodium Bisulfite; Acetylated Lanolin Alcohols; Acetylated Monoglycerides; Acetylcysteine; Acetyltryptophan, DL-; Acrylates Copolymer; Acrylic Acid-Isooctyl Acrylate Copolymer; Acrylic Adhesive 788; Activated Charcoal; Adcote 72A 103; Adhesive Tape; Adipic Acid; Aerotex Resin 3730; Alanine; Albumin Aggregated; Albumin Colloidal; Albumin Human; Alcohol; Alcohol, Dehydrated; Alcohol, Denatured; Alcohol, Diluted; Alfadex; Alginic Acid; Alkyl Ammonium Sulfonic Acid Betaine; Alkyl Aryl Sodium Sulfonate; Allantoin; Allyl .Alpha.-Ionone; Almond Oil; Alpha-Terpineol; Alpha-Tocopherol; Alpha-Tocopherol Acetate, D1-; Alpha-Tocopherol, D1-; Aluminum Acetate; Aluminum Chlorhydroxy Allantoinate; Aluminum Hydroxide; Aluminum Hydroxide-Sucrose, Hydrated; Aluminum Hydroxide Gel; Aluminum Hydroxide Gel F 500; Aluminum Hydroxide Gel F 5000. Aluminum Monostearate; Aluminum Oxide; Aluminum Polyester; Aluminum Silicate; Aluminum Starch Octenylsuccinate; Aluminum Stearate; Aluminum Subacetate; Aluminum Sulfate Anhydrous; Amerchol C; Amerchol-Cab; Aminomethylpropanol; Ammonia; Ammonia Solution; Ammonia Solution, Strong; Ammonium Acetate; Ammonium Hydroxide; Ammonium Lauryl Sulfate; Ammonium Nonoxynol-4 Sulfate; Ammonium Salt Of C-12-C-15 Linear Primary Alcohol Ethoxylate; Ammonium Sulfate; Ammonyx; Amphoteric-2; Amphoteric-9; Anethole; Anhydrous Citric Acid; Anhydrous Dextrose; Anhydrous Lactose; Anhydrous Trisodium Citrate; Aniseed Oil; Anoxid Sbn; Antifoam; Antipyrine; Apaflurane; Apricot Kernel Oil Peg-6 Esters; Aquaphor; Arginine; Arlacel; Ascorbic Acid; Ascorbyl Palmitate; Aspartic Acid; Balsam Peru; Barium Sulfate; Beeswax; Beeswax, Synthetic; Beheneth-10; Bentonite; Benzalkonium Chloride; Benzenesulfonic Acid; Benzethonium Chloride; Benzododecinium Bromide; Benzoic Acid; Benzyl Alcohol; Benzyl Benzoate; Benzyl Chloride; Betadex; Bibapcitide; Bismuth Subgallate; Boric Acid; Brocrinat; Butane; Butyl Alcohol; Butyl Ester Of Vinyl Methyl Ether/Maleic Anhydride Copolymer (125000 Mw); Butyl Stearate; Butylated Hydroxyanisole; Butylated Hydroxytoluene; Butylene Glycol; Butylparaben; Butyric Acid; C20-40 Pareth-24; Caffeine; Calcium; Calcium Carbonate; Calcium Chloride; Calcium Gluceptate; Calcium Hydroxide; Calcium Lactate; Calcobutrol; Caldiamide Sodium; Caloxetate Trisodium; Calteridol Calcium, Canada Balsam; Caprylic/Capric Triglyceride; Caprylic/Capric/Stearic Triglyceride; Captan; Captisol; Caramel; Carbomer 1342; Carbomer 1382; Carbomer 934; Carbomer 934p; Carbomer 940; Carbomer 941; Carbomer 980; Carbomer 981; Carbomer Homopolymer Type B (Allyl Pentaerythritol Crosslinked); Carbomer Homopolymer Type C (Allyl Pentaerythritol Crosslinked); Carbon Dioxide; Carboxy Vinyl Copolymer; Carboxymethylcellulose; Carboxymethylcellulose Sodium; Carboxypolymethylene; Carrageenan; Carrageenan Salt; Castor Oil; Cedar Leaf Oil; Cellulose; Cellulose, Microcrystalline; Cerasynt-Se; Ceresin; Ceteareth-12; Ceteareth-15; Ceteareth-30; Cetearyl Alcohol/Ceteareth-20; Cetearyl Ethylhexanoate; Ceteth-10; Ceteth-2; Ceteth-20; Ceteth-23; Cetostearyl Alcohol; Cetrimonium Chloride; Cetyl Alcohol; Cetyl Esters Wax; Cetyl Palmitate; Cetylpyridinium Chloride; Chlorobutanol; Chlorobutanol Hemihydrate; Chlorobutanol, Anhydrous; Chlorocresol; Chloroxylenol; Cholesterol; Choleth; Choleth-24; Citrate; Citric Acid; Citric Acid Monohydrate; Citric Acid, Hydrous; Cocamide Ether Sulfate; Cocamine Oxide; Coco Betaine; Coco Diethanolamide; Coco Monoethanolamide; Cocoa Butter; Coco-Glycerides; Coconut Oil, Coconut Oil, Hydrogenated; Coconut Oil/Palm Kernel Oil Glycerides. Hydrogenated; Cocoyl Caprylocaprate; Cola Nitida Seed Extract; Collagen; Coloring Suspension; Corn Oil; Cottonseed Oil; Cream Base; Creatine; Creatinine; Cresol; Croscarmellose Sodium; Crospovidone; Cupric Sulfate; Cupric Sulfate Anhydrous; Cyclomethicone; Cyclomethicone/Dimethicone Copolyol; Cysteine; Cysteine Hydrochloride; Cysteine Hydrochloride Anhydrous; Cysteine, D1-; D&C Red No. 28; D&C Red No. 33; D&C Red No. 36; D&C Red No. 39; D&C Yellow No. 10; Dalfampridine; Daubert 1-5 Pestr (Matte) 164z; Decyl Methyl Sulfoxide; Dehydag Wax Sx; Dehydroacetic Acid; Dehymuls E; Denatonium Benzoate; Deoxycholic Acid; Dextran; Dextran 40; Dextrin; Dextrose; Dextrose Monohydrate; Dextrose Solution; Diatrizoic Acid; Diazolidinyl Urea; Dichlorobenzyl Alcohol; Dichlorodifluoromethane; Dichlorotetrafluoroethane; Diethanolamine; Diethyl Pyrocarbonate; Diethyl Sebacate; Diethylene Glycol Monoethyl Ether; Diethylhexyl Phthalate; Dihydroxyaluminum Aminoacetate; Diisopropanolamine, Diisopropyl Adipate; Diisopropyl Dilinoleate; Dimethicone 350; Dimethicone Copolyol; Dimethicone Mdx4-4210; Dimethicone Medical Fluid 360; Dimethyl Isosorbide; Dimethyl Sulfoxide; Dimethylaminoethyl Methacrylate-Butyl Methacrylate-Methyl Methacrylate Copolymer; Dimethyldioctadecylammonium Bentonite; Dimethylsiloxane/Methylvinylsiloxane Copolymer; Dinoseb Ammonium Salt; Dipalmitoylphosphatidylglycerol, D1-; Dipropylene Glycol; Disodium Cocoamphodiacetate; Disodium Laureth Sulfosuccinate; Disodium Lauryl Sulfosuccinate; Disodium Sulfosalicylate; Disofenin; Divinylbenzene Styrene Copolymer; Dmdm Hydantoin; Docosanol; Docusate Sodium; Duro-Tak 280-2516; Duro-Tak 387-2516; Duro-Tak 80-1196; Duro-Tak 87-2070; Duro-Tak 87-2194; Duro-Tak 87-2287; Duro-Tak 87-2296; Duro-Tak 87-2888; Duro-Tak 87-2979; Edetate Calcium Disodium; Edetate Disodium; Edetate Disodium Anhydrous; Edetate Sodium; Edetic Acid; Egg Phospholipids; Entsufon; Entsufon Sodium; Epilactose; Epitetracycline Hydrochloride; Essence Bouquet 9200; Ethanolamine Hydrochloride; Ethyl Acetate; Ethyl Oleate; Ethylcelluloses; Ethylene Glycol; Ethylene Vinyl Acetate Copolymer; Ethylenediamine; Ethylenediamine Dihydrochloride; Ethylene-Propylene Copolymer; Ethylene-Vinyl Acetate Copolymer (28% Vinyl Acetate); Ethylene-Vinyl Acetate Copolymer (9% Vinylacetate); Ethylhexyl Hydroxystearate; Ethylparaben; Eucalyptol; Exametazime; Fat, Edible; Fat, Hard; Fatty Acid Esters; Fatty Acid Pentaerythriol Ester; Fatty Acids; Fatty Alcohol Citrate; Fatty Alcohols; Fd&C Blue No. 1; Fd&C Green No. 3; Fd&C Red No. 4; Fd&C Red No. 40; Fd&C Yellow No. 10 (Delisted); Fd&C Yellow No. 5; Fd&C Yellow No. 6; Ferric Chloride; Ferric Oxide; Flavor 89-186; Flavor 89-259; Flavor Df-119; Flavor Df-1530; Flavor Enhancer; Flavor FIG. 827118; Flavor Raspberry Pfc-8407; Flavor Rhodia Pharmaceutical No. Rf 451; Fluorochlorohydrocarbons; Formaldehyde; Formaldehyde Solution; Fractionated Coconut Oil; Fragrance 3949-5; Fragrance 520a; Fragrance 6.007; Fragrance 91-122; Fragrance 9128-Y; Fragrance 93498g; Fragrance Balsam Pine No. 5124; Fragrance Bouquet 10328; Fragrance Chemoderm 6401-B; Fragrance Chemoderm 6411. Fragrance Cream No. 73457; Fragrance Cs-28197; Fragrance Felton 066m; Fragrance Firmenich 47373; Fragrance Givaudan Ess 9090/1c; Fragrance H-6540; Fragrance Herbal 10396; Fragrance Nj-1085; Fragrance P O F1-147; Fragrance Pa 52805; Fragrance Pera Derm D; Fragrance Rbd-9819; Fragrance Shaw Mudge U-7776; Fragrance Tf 044078; Fragrance Ungerer Honeysuckle K 2771; Fragrance Ungerer N5195; Fructose; Gadolinium Oxide; Galactose; Gamma Cyclodextrin; Gelatin; Gelatin, Crosslinked; Gelfoam Sponge; Gellan Gum (Low Acyl); Gelva 737; Gentisic Acid; Gentisic Acid Ethanolamide; Gluceptate Sodium; Gluceptate Sodium Dihydrate; Gluconolactone; Glucuronic Acid; Glutamic Acid, D1-; Glutathione; Glycerin; Glycerol Ester Of Hydrogenated Rosin; Glyceryl Citrate; Glyceryl Isostearate; Glyceryl Laurate; Glyceryl Monostearate; Glyceryl Oleate; Glyceryl Oleate/Propylene Glycol; Glyceryl Palmitate; Glyceryl Ricinoleate; Glyceryl Stearate; Glyceryl Stearate-Laureth-23; Glyceryl Stearate/Peg Stearate; Glyceryl Stearate/Peg-100 Stearate; Glyceryl Stearate/Peg-40 Stearate; Glyceryl Stearate-Stearamidoethyl Diethylamine; Glyceryl Trioleate; Glycine; Glycine Hydrochloride; Glycol Distearate; Glycol Stearate; Guanidine Hydrochloride; Guar Gum; Hair Conditioner (18n195-1m); Heptane; Hetastarch; Hexylene Glycol; High Density Polyethylene; Histidine; Human Albumin Microspheres; Hyaluronate Sodium; Hydrocarbon; Hydrocarbon Gel, Plasticized; Hydrochloric Acid; Hydrochloric Acid, Diluted; Hydrocortisone; Hydrogel Polymer; Hydrogen Peroxide; Hydrogenated Castor Oil; Hydrogenated Palm Oil; Hydrogenated Palm/Palm Kernel Oil Peg-6 Esters; Hydrogenated Polybutene 635-690; Hydroxide Ion; Hydroxyethyl Cellulose; Hydroxyethylpiperazine Ethane Sulfonic Acid; Hydroxymethyl Cellulose; Hydroxyoctacosanyl Hydroxystearate; Hydroxypropyl Cellulose; Hydroxypropyl Methylcellulose 2906; Hydroxypropyl-Beta-cyclodextrin; Hypromellose 2208 (15000 Mpa.S); Hypromellose 2910 (15000 Mpa.S); Hypromelloses; Imidurea; Iodine; Iodoxamic Acid; Iofetamine Hydrochloride; Irish Moss Extract; Isobutane; Isoceteth-20; Isoleucine; Isooctyl Acrylate; Isopropyl Alcohol; Isopropyl Isostearate; Isopropyl Myristate; Isopropyl Myristate-Myristyl Alcohol; Isopropyl Palmitate; Isopropyl Stearate; Isostearic Acid; Isostearyl Alcohol; Isotonic Sodium Chloride Solution; Jelene; Kaolin; Kathon Cg; Kathon Cg II; Lactate; Lactic Acid; Lactic Acid, D1-; Lactic Acid, L-; Lactobionic Acid; Lactose; Lactose Monohydrate; Lactose. Hydrous; Laneth; Lanolin; Lanolin Alcohol-Mineral Oil; Lanolin Alcohols; Lanolin Anhydrous; Lanolin Cholesterols; Lanolin Nonionic Derivatives; Lanolin, Ethoxylated; Lanolin, Hydrogenated; Lauralkonium Chloride; Lauramine Oxide; Laurdimonium Hydrolyzed Animal Collagen; Laureth Sulfate; Laureth-2; Laureth-23; Laureth-4; Lauric Diethanolamide; Lauric Myristic Diethanolamide; Lauroyl Sarcosine; Lauryl Lactate; Lauryl Sulfate; Lavandula Angustifolia Flowering Top; Lecithin; Lecithin Unbleached; Lecithin. Egg; Lecithin, Hydrogenated; Lecithin, Hydrogenated Soy; Lecithin, Soybean, Lemon Oil; Leucine; Levulinic Acid; Lidofenin; Light Mineral Oil; Light Mineral Oil (85 Ssu); Limonene, (+/−)-; Lipocol Sc-15; Lysine; Lysine Acetate; Lysine Monohydrate; Magnesium Aluminum Silicate; Magnesium Aluminum Silicate Hydrate; Magnesium Chloride; Magnesium Nitrate; Magnesium Stearate; Maleic Acid; Mannitol; Maprofix; Mebrofenin; Medical Adhesive Modified S-15; Medical Antiform A-F Emulsion; Medronate Disodium; Medronic Acid; Meglumine; Menthol; Metacresol; Metaphosphoric Acid; Methanesulfonic Acid; Methionine; Methyl Alcohol; Methyl Gluceth-10; Methyl Gluceth-20, Methyl Gluceth-20 Sesquistearate; Methyl Glucose Sesquistearate; Methyl Laurate; Methyl Pyrrolidone; Methyl Salicylate; Methyl Stearate; Methylboronic Acid; Methylcellulose (4000 Mpa.S); Methylcelluloses; Methylchloroisothiazolinone; Methylene Blue; Methylisothiazolinone; Methylparaben; Microcrystalline Wax; Mineral Oil; Mono And Diglyceride; Monostearyl Citrate; Monothioglycerol; Multisterol Extract; Myristyl Alcohol; Myristyl Lactate; Myristyl-.Gamma.-Picolinium Chloride; N-(Carbamoyl-Methoxy Peg-40)-1,2-Distearoyl-Cephalin Sodium; N,N-Dimethylacetamide; Niacinamide; Nioxime; Nitric Acid; Nitrogen; Nonoxynol Iodine; Nonoxynol-15; Nonoxynol-9; Norflurane; Oatmeal; Octadecene-1/Maleic Acid Copolymer; Octanoic Acid; Octisalate; Octoxynol-1; Octoxynol-40; Octoxynol-9; Octyldodecanol; Octylphenol Polymethylene; Oleic Acid; Oleth-10/Oleth-5; Oleth-2; Oleth-20; Oleyl Alcohol; Oleyl Oleate; Olive Oil; Oxidronate Disodium; Oxyquinoline; Palm Kernel Oil; Palmitamine Oxide; Parabens; Paraffin; Paraffin, White Soft; Parfum Creme 45/3; Peanut Oil; Peanut Oil, Refined; Pectin; Peg 6-32 Stearate/Glycol Stearate; Peg Vegetable Oil; Peg-100 Stearate; Peg-12 Glyceryl Laurate; Peg-120 Glyceryl Stearate; Peg-120 Methyl Glucose Dioleate; Peg-15 Cocamine; Peg-150 Distearate; Peg-2 Stearate; Peg-20 Sorbitan Isostearate; Peg-22 Methyl Ether/Dodecyl Glycol Copolymer; Peg-25 Propylene Glycol Stearate; Peg-4 Dilaurate; Peg-4 Laurate; Peg-40 Castor Oil; Peg-40 Sorbitan Diisostearate; Peg-45/Dodecyl Glycol Copolymer; Peg-5 Oleate; Peg-50 Stearate; Peg-54 Hydrogenated Castor Oil; Peg-6 Isostearate; Peg-60 Castor Oil; Peg-60 Hydrogenated Castor Oil; Peg-7 Methyl Ether; Peg-75 Lanolin; Peg-8 Laurate; Peg-8 Stearate; Pegoxol 7 Stearate; Pentadecalactone; Pentaerythritol Cocoate; Pentasodium Pentetate; Pentetate Calcium Trisodium; Pentetic Acid; Peppermint Oil; Perflutren; Perfume 25677; Perfume Bouquet; Perfume E-1991; Perfume Gd 5604; Perfume Tana 90/42 Scba; Perfume W-1952-1; Petrolatum; Petrolatum, White; Petroleum Distillates; Phenol; Phenol, Liquefied; Phenonip; Phenoxyethanol; Phenylalanine; Phenylethyl Alcohol; Phenylmercuric Acetate; Phenylmercuric Nitrate; Phosphatidyl Glycerol, Egg; Phospholipid; Phospholipid, Egg; Phospholipon 90 g; Phosphoric Acid; Pine Needle Oil (Pinus Sylvestris); Piperazine Hexahydrate; Plastibase-50w; Polacrilin; Polidronium Chloride; Poloxamer 124; Poloxamer 181; Poloxamer 182; Poloxamer 188; Poloxamer 237; Poloxamer 407; Poly(Bis(P-Carboxyphenoxy)Propane Anhydride):Sebacic Acid; Poly(Dimethylsiloxane/Methylvinylsiloxane/Methylhydrogensiloxane) Dimethylvinyl Or Dimethylhydroxy Or Trimethyl Endblocked; Poly(Dl-Lactic-Co-Glycolic Acid), (50:50; Poly(Dl-Lactic-Co-Glycolic Acid), Ethyl Ester Terminated. (50:50; Polyacrylic Acid (250000 Mw); Polybutene (1400 Mw); Polycarbophil; Polyester; Polyester Polyamine Copolymer; Polyester Rayon; Polyethylene Glycol 1000; Polyethylene Glycol 1450; Polyethylene Glycol 1500; Polyethylene Glycol 1540; Polyethylene Glycol 200; Polyethylene Glycol 300; Polyethylene Glycol 300-1600; Polyethylene Glycol 3350; Polyethylene Glycol 400; Polyethylene Glycol 4000; Polyethylene Glycol 540; Polyethylene Glycol 600; Polyethylene Glycol 6000; Polyethylene Glycol 8000; Polyethylene Glycol 900; Polyethylene High Density Containing Ferric Oxide Black (<1%); Polyethylene Low Density Containing Barium Sulfate (20-24%); Polyethylene T; Polyethylene Terephthalates; Polyglactin; Polyglyceryl-3 Oleate; Polyglyceryl-4 Oleate; Polyhydroxyethyl Methacrylate; Polyisobutylene; Polyisobutylene (1100000 Mw); Polyisobutylene (35000 Mw); Polyisobutylene 178-236; Polyisobutylene 241-294; Polyisobutylene 35-39; Polyisobutylene Low Molecular Weight; Polyisobutylene Medium Molecular Weight; Polyisobutylene/Polybutene Adhesive; Polylactide; Polyols; Polyoxyethylene-Polyoxypropylene 1800; Polyoxyethylene Alcohols; Polyoxyethylene Fatty Acid Esters; Polyoxyethylene Propylene; Polyoxyl 20 Cetostearyl Ether; Polyoxyl 35 Castor Oil; Polyoxyl 40 Hydrogenated Castor Oil; Polyoxyl 40 Stearate; Polyoxyl 400 Stearate; Polyoxyl 6 And Polyoxyl 32 Palmitostearate; Polyoxyl Distearate; Polyoxyl Glyceryl Stearate; Polyoxyl Lanolin; Polyoxyl Palmitate; Polyoxyl Stearate; Polypropylene; Polypropylene Glycol; Polyquaternium-10; Polyquaternium-7 (70/30 Acrylamide/Dadmac; Polysiloxane; Polysorbate 20; Polysorbate 40; Polysorbate 60; Polysorbate 65; Polysorbate 80; Polyurethane; Polyvinyl Acetate; Polyvinyl Alcohol; Polyvinyl Chloride; Polyvinyl Chloride-Polyvinyl Acetate Copolymer; Polyvinylpyridine; Poppy Seed Oil; Potash; Potassium Acetate; Potassium Alum; Potassium Bicarbonate; Potassium Bisulfite; Potassium Chloride; Potassium Citrate; Potassium Hydroxide; Potassium Metabisulfite; Potassium Phosphate, Dibasic; Potassium Phosphate, Monobasic; Potassium Soap; Potassium Sorbate; Povidone Acrylate Copolymer; Povidone Hydrogel; Povidone K17; Povidone K25; Povidone K29/32; Povidone K30; Povidone K90; Povidone K90f; Povidone/Eicosene Copolymer; Povidones; Ppg-12/Smdi Copolymer; Ppg-15 Stearyl Ether; Ppg-20 Methyl Glucose Ether Distearate; Ppg-26 Oleate; Product Wat; Proline; Promulgen D; Promulgen G; Propane; Propellant A-46; Propyl Gallate; Propylene Carbonate; Propylene Glycol; Propylene Glycol Diacetate; Propylene Glycol Dicaprylate; Propylene Glycol Monolaurate; Propylene Glycol Monopalmitostearate; Propylene Glycol Palmitostearate; Propylene Glycol Ricinoleate; Propylene Glycol/Diazolidinyl; Urea/Methylparaben/Propylparben; Propylparaben; Protamine Sulfate; Protein Hydrolysate; Pvm/Ma Copolymer; Quaternium-15; Quaternium-15 Cis-Form; Quaternium-52; Ra-2397; Ra-3011; Saccharin; Saccharin Sodium; Saccharin Sodium Anhydrous; Safflower Oil; Sd Alcohol 3a; Sd Alcohol 40; Sd Alcohol 40-2; Sd Alcohol 40b; Sepineo P 600; Serine; Sesame Oil; Shea Butter; Silastic Brand Medical Grade Tubing; Silastic Medical Adhesive, Silicone Type A; Silica. Dental; Silicon; Silicon Dioxide; Silicon Dioxide, Colloidal; Silicone; Silicone Adhesive 4102; Silicone Adhesive 4502; Silicone Adhesive Bio-Psa Q7-4201; Silicone Adhesive Bio-Psa Q7-4301; Silicone Emulsion; Silicone/Polyester Film Strip; Simethicone; Simethicone Emulsion; Sipon Ls 20np; Soda Ash; Sodium Acetate; Sodium Acetate Anhydrous; Sodium Alkyl Sulfate; Sodium Ascorbate; Sodium Benzoate; Sodium Bicarbonate; Sodium Bisulfate; Sodium Bisulfite; Sodium Borate; Sodium Borate Decahydrate; Sodium Carbonate; Sodium Carbonate Decahydrate; Sodium Carbonate Monohydrate; Sodium Cetostearyl Sulfate; Sodium Chlorate; Sodium Chloride; Sodium Chloride Injection; Sodium Chloride Injection, Bacteriostatic; Sodium Cholesteryl Sulfate; Sodium Citrate; Sodium Cocoyl Sarcosinate; Sodium Desoxycholate; Sodium Dithionite; Sodium Dodecylbenzenesulfonate; Sodium Formaldehyde Sulfoxylate; Sodium Gluconate; Sodium Hydroxide; Sodium Hypochlorite; Sodium Iodide; Sodium Lactate; Sodium Lactate, L-; Sodium Laureth-2 Sulfate; Sodium Laureth-3 Sulfate; Sodium Laureth-5 Sulfate; Sodium Lauroyl Sarcosinate; Sodium Lauryl Sulfate; Sodium Lauryl Sulfoacetate; Sodium Metabisulfite; Sodium Nitrate; Sodium Phosphate; Sodium Phosphate Dihydrate; Sodium Phosphate, Dibasic; Sodium Phosphate, Dibasic, Anhydrous; Sodium Phosphate, Dibasic, Dihydrate; Sodium Phosphate, Dibasic, Dodecahydrate; Sodium Phosphate, Dibasic, Heptahydrate; Sodium Phosphate, Monobasic; Sodium Phosphate, Monobasic, Anhydrous; Sodium Phosphate. Monobasic, Dihydrate: Sodium Phosphate, Monobasic, Monohydrate; Sodium Polyacrylate (2500000 Mw); Sodium Pyrophosphate; Sodium Pyrrolidone Carboxylate; Sodium Starch Glycolate; Sodium Succinate Hexahydrate; Sodium Sulfate; Sodium Sulfate Anhydrous; Sodium Sulfate Decahydrate; Sodium Sulfite; Sodium Sulfosuccinated Undecyclenic Monoalkylolamide; Sodium Tartrate; Sodium Thioglycolate; Sodium Thiomalate; Sodium Thiosulfate; Sodium Thiosulfate Anhydrous; Sodium Trimetaphosphate; Sodium Xylenesulfonate; Somay 44; Sorbic Acid; Sorbitan; Sorbitan Isostearate; Sorbitan Monolaurate; Sorbitan Monooleate; Sorbitan Monopalmitate; Sorbitan Monostearate; Sorbitan Sesquioleate; Sorbitan Trioleate; Sorbitan Tristearate; Sorbitol; Sorbitol Solution; Soybean Flour; Soybean Oil; Spearmint Oil; Spermaceti; Squalane; Stabilized Oxychloro Complex; Stannous 2-Ethylhexanoate; Stannous Chloride; Stannous Chloride Anhydrous; Stannous Fluoride; Stannous Tartrate; Starch; Starch 1500, Pregelatinized; Starch, Corn; Stearalkonium Chloride; Stearalkonium Hectorite/Propylene Carbonate; Stearamidoethyl Diethylamine; Steareth-10; Steareth-100; Steareth-2; Steareth-20; Steareth-21; Steareth-40; Stearic Acid; Stearic Diethanolamide; Stearoxytrimethylsilane; Steartrimonium Hydrolyzed Animal Collagen; Stearyl Alcohol; Sterile Water For Inhalation; Styrene/Isoprene/Styrene Block Copolymer; Succimer; Succinic Acid; Sucralose; Sucrose; Sucrose Distearate; Sucrose Polyesters; Sulfacetamide Sodium; Sulfobutylether .Beta.-Cyclodextrin; Sulfur Dioxide; Sulfuric Acid; Sulfurous Acid; Surfactol Qs; Tagatose. D-; Talc, Tall Oil; Tallow Glycerides; Tartaric Acid; Tartaric Acid, D1-; Tenox; Tenox-2; Tert-Butyl Alcohol; Tert-Butyl Hydroperoxide; Tert-Butylhydroquinone; Tetrakis(2-Methoxyisobutylisocyanide)Copper(I) Tetrafluoroborate; Tetrapropyl Orthosilicate; Tetrofosmin; Theophylline; Thimerosal; Threonine; Thymol; Tin; Titanium Dioxide; Tocopherol; Tocophersolan; Total parenteral nutrition, lipid emulsion; Triacetin; Tricaprylin; Trichloromonofluoromethane; Trideceth-10; Triethanolamine Lauryl Sulfate; Trifluoroacetic Acid; Triglycerides, Medium Chain; Trihydroxystearin; Trilaneth-4 Phosphate; Trilaureth-4 Phosphate; Trisodium Citrate Dihydrate; Trisodium Hedta; Triton 720; Triton X-200; Trolamine; Tromantadine; Tromethamine (TRIS); Tryptophan; Tyloxapol; Tyrosine; Undecylenic Acid; Union 76 Amsco-Res 6038; Urea; Valine; Vegetable Oil; Vegetable Oil Glyceride, Hydrogenated; Vegetable Oil, Hydrogenated; Versetamide; Viscarin; Viscose/Cotton; Vitamin E; Wax, Emulsifying; Wecobee Fs; White Ceresin Wax; White Wax; Xanthan Gum; Zinc; Zinc Acetate; Zinc Carbonate; Zinc Chloride; and Zinc Oxide.

Pharmaceutical formulations of AAV particles disclosed herein may include cations or anions. In certain embodiments, the formulations include metal cations such as, but not limited to, Zn2+, Ca2+, Cu2+, Mn2+, Mg+ and combinations thereof. As a non-limiting example, formulations may include polymers and complexes with a metal cation (See e.g., U.S. Pat. Nos. 6,265,389 and 6,555,525, each of which is herein incorporated by reference in its entirety).

Formulations of the present disclosure may also include one or more pharmaceutically acceptable salts. As used herein, “pharmaceutically acceptable salts” refers to derivatives of the disclosed compounds wherein the parent compound is modified by converting an existing acid or base moiety to its salt form (e.g., by reacting the free base group with a suitable organic acid).

In certain embodiments, additional excipients that may be used in formulating the pharmaceutical composition may include magnesium chloride (MgCl2), arginine, sorbitol, and/or trehalose.

Formulations of the present disclosure may include at least one excipient and/or diluent in addition to the AAV particle. The formulation may include 1, 2, 3, 4, 5, 6, 7, 8, 9, 10, or more than 10 excipients and/or diluents in addition to the AAV particle.

In certain embodiments, the formulation may include, but is not limited to, phosphate-buffered saline (PBS). As a non-limiting example, the PBS may include sodium chloride, potassium chloride, disodium phosphate, monopotassium phosphate, and distilled water. In some instances, the PBS does not contain potassium or magnesium. In other instances, the PBS contains calcium and magnesium.

### Sodium Phosphate

In certain embodiments, at least one of the components in the formulation is sodium phosphate. The formulation may include monobasic, dibasic or a combination of both monobasic and dibasic sodium phosphate.

In certain embodiments, the concentration of sodium phosphate in a formulation may be, but is not limited to, 0.1 mM, 0.2 mM, 0.3 mM, 0.4 mM, 0.5 mM, 0.6 mM, 0.7 mM, 0.8 mM, 0.9 mM, 1 mM, 1.1 mM, 1.2 mM, 1.3 mM, 1.4 mM, 1.5 mM, 1.6 mM, 1.7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.1 mM, 4.2 mM, 4.3 mM, 4.4 mM, 4.5 mM, 4.6 mM, 4.7 mM, 4.8 mM, 4.9 mM, 5 mM, 5.1 mM, 5.2 mM, 5.3 mM, 5.4 mM, 5.5 mM, 5.6 mM, 5.7 mM, 5.8 mM, 5.9 mM, 6 mM, 6.1 mM, 6.2 mM, 6.3 mM, 6.4 mM, 6.5 mM, 6.6 mM, 6.7 mM, 6.8 mM, 6.9 mM, 7 mM, 7.1 mM, 7.2 mM, 7.3 mM, 7.4 mM, 7.5 mM, 7.6 mM, 7.7 mM, 7.8 mM, 7.9 mM, 8 mM, 8.1 mM, 8.2 mM, 8.3 mM, 8.4 mM, 8.5 mM, 8.6 mM, 8.7 mM, 8.8 mM, 8.9 mM, 9 mM, 9.1 mM, 9.2 mM, 9.3 mM, 9.4 mM, 9.5 mM, 9.6 mM, 9.7 mM, 9.8 mM, 9.9 mM, 10 mM, 10.1 mM, 10.2 mM, 10.3 mM, 10.4 mM, 10.5 mM, 10.6 mM, 10.7 mM, 10.8 mM, 10.9 mM, 11 mM, 11.1 mM, 11.2 mM, 11.3 mM, 11.4 mM, 11.5 mM, 11.6 mM, 11.7 mM, 11.8 mM, 11.9 mM, 12 mM, 12.1 mM, 12.2 mM, 12.3 mM, 12.4 mM, 12.5 mM, 12.6 mM, 12.7 mM, 12.8 mM, 12.9 mM, 13 mM, 13.1 mM, 13.2 mM, 13.3 mM, 13.4 mM, 13.5 mM, 13.6 mM, 13.7 mM, 13.8 mM, 13.9 mM, 14 mM, 14.1 mM, 14.2 mM, 14.3 mM, 14.4 mM 14.5 mM, 14.6 mM, 14.7 mM, 14.8 mM, 14.9 mM or 15 mM.

The formulation may include sodium phosphate in a range of 0-0.5 mM, 0.1-0.6 mM, 0.2-0.7 mM, 0.3-0.8 mM, 0.4-0.9 mM, 0.5-1 mM, 0.6-1.1 mM, 0.7-1.2 mM, 0.8-1.3 mM, 0.9-1.4 mM, 1-1.5 mM, 1.1-1.6 mM, 1.2-1.7 mM, 1.3-1.8 mM, 1.4-1.9 mM, 1.5-2 mM, 1.6-2.1 mM, 1.7-2.2 mM, 1.8-2.3 mM, 1.9-2.4 mM, 2-2.5 mM, 2.1-2.6 mM, 2.2-2.7 mM, 2.3-2.8 mM, 2.4-2.9 mM, 2.5-3 mM, 2.6-3.1 mM, 2.7-3.2 mM, 2.8-3.3 mM, 2.9-3.4 mM, 3-3.5 mM, 3.1-3.6 mM, 3.2-3.7 mM, 3.3-3.8 mM, 3.4-3.9 mM, 3.5-4 mM, 3.6-4.1 mM, 3.7-4.2 mM, 3.8-4.3 mM, 3.9-4.4 mM, 4-4.5 mM, 4.1-4.6 mM, 4.2-4.7 mM, 4.3-4.8 mM, 4.4-4.9 mM, 4.5-5 mM, 4.6-5.1 mM, 4.7-5.2 mM, 4.8-5.3 mM, 4.9-5.4 mM, 5-5.5 mM, 5.1-5.6 mM, 5.2-5.7 mM, 5.3-5.8 mM, 5.4-5.9 mM, 5.5-6 mM, 5.6-6.1 mM, 5.7-6.2 mM, 5.8-6.3 mM, 5.9-6.4 mM, 6-6.5 mM, 6.1-6.6 mM, 6.2-6.7 mM, 6.3-6.8 mM, 6.4-6.9 mM, 6.5-7 mM, 6.6-7.1 mM, 6.7-7.2 mM, 6.8-7.3 mM, 6.9-7.4 mM, 7-7.5 mM, 7.1-7.6 mM, 7.2-7.7 mM, 7.3-7.8 mM, 7.4-7.9 mM, 7.5-8 mM, 7.6-8.1 mM, 7.7-8.2 mM, 7.8-8.3 mM, 7.9-8.4 mM, 8-8.5 mM, 8.1-8.6 mM, 8.2-8.7 mM, 8.3-8.8 mM, 8.4-8.9 mM, 8.5-9 mM, 8.6-9.1 mM, 8.7-9.2 mM, 8.8-9.3 mM, 8.9-9.4 mM, 9-9.5 mM, 9.1-9.6 mM, 9.2-9.7 mM, 9.3-9.8 mM, 9.4-9.9 mM, 9.5-10 mM, 9.6-10.1 mM, 9.7-10.2 mM, 9.8-10.3 mM, 9.9-10.4 mM, 10-10.5 mM, 10.1-10.6 mM, 10.2-10.7 mM, 10.3-10.8 mM, 10.4-10.9 mM, 10.5-11 mM, 10.6-11.1 mM, 10.7-11.2 mM, 10.8-11.3 mM, 10.9-11.4 mM, 11-11.5 mM, 11.1-11.6 mM, 11.2-11.7 mM, 11.3-11.8 mM, 11.4-11.9 mM, 11.5-12 mM, 11.6-12.1 mM, 11.7-12.2 mM, 11.8-12.3 mM, 11.9-12.4 mM, 12-12.5 mM, 12.1-12.6 mM, 12.2-12.7 mM, 12.3-12.8 mM, 12.4-12.9 mM, 12.5-13 mM, 12.6-13.1 mM, 12.7-13.2 mM, 12.8-13.3 mM, 12.9-13.4 mM, 13-13.5 mM, 13.1-13.6 mM, 13.2-13.7 mM, 13.3-13.8 mM, 13.4-13.9 mM, 13.5-14 mM, 13.6-14.1 mM, 13.7-14.2 mM, 13.8-14.3 mM, 13.9-14.4 mM, 14-14.5 mM, 14.1-14.6 mM, 14.2-14.7 mM, 14.3-14.8 mM, 14.4-14.9 mM, 14.5-15 mM, 0-1 mM, 1-2 mM, 2-3 mM, 3-4 mM, 4-5 mM, 5-6 mM, 6-7 mM, 7-8 mM, 8-9 mM, 9-10 mM 10-11 mM, 11-12 mM, 12-13 mM, 13-14 mM, 14-15 mM, 15-16 mM, 0-2 mM, 1-3 mM, 24 mM, 3-5 mM, 4-6 mM, 5-7 mM, 6-8 mM, 7-9 mM, 8-10 mM, 9-11 mM, 10-12 mM, 11-13 mM, 12-14 mM, 13-15 mM, 0-3 mM, 1-4 mM, 2-5 mM, 3-6 mM, 4-7 mM, 5-8 mM, 6-9 mM, 7-10 mM, 8-11 mM, 9-12 mM, 10-13 mM, 11-14 mM, 12-15 mM, 0-4 mM, 1-5 mM, 2-6 mM, 3-7 mM, 4-8 mM, 5-9 mM, 6-10 mM, 7-11 mM, 8-12 mM, 9-13 mM, 10-14 mM, 11-15 mM, 0-5 mM, 1-6 mM, 2-7 mM, 3-8 mM, 4-9 mM, 5-10 mM, 6-11 mM, 7-12 mM, 8-13 mM, 9-14 mM, 10-15 mM, 0-6 mM, 1-7 mM, 2-8 mM, 3-9 mM, 4-10 mM, 5-11 mM, 6-12 mM, 7-13 mM, 8-14 mM, 9-15 mM, 0-7 mM, 1-8 mM, 2-9 mM, 3-10 mM, 4-11 mM, 5-12 mM, 6-13 mM, 7-14 mM, 8-15 mM, 0-8 mM, 1-9 mM, 2-10 mM, 3-11 mM, 4-12 mM, 5-13 mM, 6-14 mM, 7-15 mM, 0-9 mM, 1-10 mM, 2-11 mM, 3-12 mM, 4-13 mM, 5-14 mM, 6-15 mM, 0-10 mM, 1-11 mM, 2-12 mM, 3-13 mM, 4-14 mM, 5-15 mM, 0-11 mM, 1-12 mM, 2-13 mM, 3-14 mM, 4-15 mM, 0-12 mM, 1-13 mM, 2-14 mM, 3-15 mM, 0-13 mM, 1-14 mM, 2-15 mM, 0-14 mM, 1-15 mM, or 0-15 mM.

In certain embodiments, the formulation may include 0-10 mM of sodium phosphate.

In certain embodiments, the formulation may include 2-12 mM of sodium phosphate.

In certain embodiments, the formulation may include 2-3 mM of sodium phosphate.

In certain embodiments, the formulation may include 9-10 mM of sodium phosphate.

In certain embodiments, the formulation may include 10-11 mM of sodium phosphate.

In certain embodiments, the formulation may include 2.7 mM of sodium phosphate.

In certain embodiments, the formulation may include 10 mM of sodium phosphate.

### Potassium Phosphate

In certain embodiments, at least one of the components in the formulation is potassium phosphate. The formulation may include monobasic, dibasic or a combination of both monobasic and dibasic potassium phosphate.

In certain embodiments, the concentration of potassium phosphate in a formulation may be, but is not limited to, 0.1 mM, 0.2 mM, 0.3 mM, 0.4 mM, 0.5 mM, 0.6 mM, 0.7 mM, 0.8 mM, 0.9 mM, 1 mM, 1.1 mM, 1.2 mM, 1.3 mM, 1.4 mM, 1.5 mM, 1.6 mM, 1.7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.1 mM, 4.2 mM, 4.3 mM, 4.4 mM, 4.5 mM, 4.6 mM, 4.7 mM, 4.8 mM, 4.9 mM, 5 mM, 5.1 mM, 5.2 mM, 5.3 mM, 5.4 mM, 5.5 mM, 5.6 mM, 5.7 mM, 5.8 mM, 5.9 mM, 6 mM, 6.1 mM, 6.2 mM, 6.3 mM, 6.4 mM, 6.5 mM, 6.6 mM, 6.7 mM, 6.8 mM, 6.9 mM, 7 mM, 7.1 mM, 7.2 mM, 7.3 mM, 7.4 mM, 7.5 mM, 7.6 mM, 7.7 mM, 7.8 mM, 7.9 mM, 8 mM, 8.1 mM, 8.2 mM, 8.3 mM, 8.4 mM, 8.5 mM, 8.6 mM, 8.7 mM, 8.8 mM, 8.9 mM, 9 mM, 9.1 mM, 9.2 mM, 9.3 mM, 9.4 mM, 9.5 mM, 9.6 mM, 9.7 mM, 9.8 mM, 9.9 mM, 10 mM, 10.1 mM, 10.2 mM, 10.3 mM, 10.4 mM, 10.5 mM, 10.6 mM, 10.7 mM, 10.8 mM, 10.9 mM, 11 mM, 11.1 mM, 11.2 mM, 11.3 mM, 11.4 mM, 11.5 mM, 11.6 mM, 11.7 mM, 11.8 mM, 11.9 mM, 12 mM, 12.1 mM, 12.2 mM, 12.3 mM, 12.4 mM, 12.5 mM, 12.6 mM 12.7 mM, 12.8 mM, 12.9 mM, 13 mM, 13.1 mM, 13.2 mM, 13.3 mM, 13.4 mM, 13.5 mM, 13.6 mM, 13.7 mM, 13.8 mM, 13.9 mM, 14 mM, 14.1 mM, 14.2 mM, 14.3 mM, 14.4 mM, 14.5 mM, 14.6 mM, 14.7 mM, 14.8 mM, 14.9 mM or 15 mM.

The formulation may include potassium phosphate in a range of 0-0.5 mM, 0.1-0.6 mM, 0.2-0.7 mM, 0.3-0.8 mM, 0.4-0.9 mM, 0.5-1 mM, 0.6-1.1 mM, 0.7-1.2 mM, 0.8-1.3 mM, 0.9-1.4 mM, 1-1.5 mM, 1.1-1.6 mM, 1.2-1.7 mM, 1.3-1.8 mM, 1.4-1.9 mM, 1.5-2 mM, 1.6-2.1 mM, 1.7-2.2 mM, 1.8-2.3 mM, 1.9-2.4 mM, 2-2.5 mM, 2.1-2.6 mM, 2.2-2.7 mM, 2.3-2.8 mM, 2.4-2.9 mM, 2.5-3 mM, 2.6-3.1 mM, 2.7-3.2 mM, 2.8-3.3 mM, 2.9-3.4 mM, 3-3.5 mM, 3.1-3.6 mM, 3.2-3.7 mM, 3.3-3.8 mM, 3.4-3.9 mM, 3.5-4 mM, 3.6-4.1 mM, 3.7-4.2 mM, 3.8-4.3 mM, 3.9-4.4 mM, 4-4.5 mM, 4.1-4.6 mM, 4.2-4.7 mM, 4.3-4.8 mM, 4.4-4.9 mM, 4.5-5 mM, 4.6-5.1 mM, 4.7-5.2 mM, 4.8-5.3 mM, 4.9-5.4 mM, 5-5.5 mM, 5.1-5.6 mM, 5.2-5.7 mM, 5.3-5.8 mM, 5.4-5.9 mM, 5.5-6 mM, 5.6-6.1 mM, 5.7-6.2 mM, 5.8-6.3 mM, 5.9-6.4 mM, 6-6.5 mM, 6.1-6.6 mM, 6.2-6.7 mM, 6.3-6.8 mM, 6.4-6.9 mM, 6.5-7 mM, 6.6-7.1 mM, 6.7-7.2 mM, 6.8-7.3 mM, 6.9-7.4 mM, 7-7.5 mM, 7.1-7.6 mM, 7.2-7.7 mM, 7.3-7.8 mM, 7.4-7.9 mM, 7.5-8 mM, 7.6-8.1 mM, 7.7-8.2 mM, 7.8-8.3 mM, 7.9-8.4 mM, 8-8.5 mM, 8.1-8.6 mM, 8.2-8.7 mM, 8.3-8.8 mM, 8.4-8.9 mM, 8.5-9 mM, 8.6-9.1 mM, 8.7-9.2 mM, 8.8-9.3 mM, 8.9-9.4 mM, 9-9.5 mM, 9.1-9.6 mM, 9.2-9.7 mM, 9.3-9.8 mM, 9.4-9.9 mM, 9.5-10 mM, 9.6-10.1 mM, 9.7-10.2 mM, 9.8-10.3 mM, 9.9-10.4 mM, 10-10.5 mM, 10.1-10.6 mM, 10.2-10.7 mM 10.3-10.8 mM, 10.4-10.9 mM, 10.5-11 mM, 10.6-11.1 mM, 10.7-11.2 mM, 10.8-11.3 mM, 10.9-11.4 mM, 11-11.5 mM, 11.1-11.6 mM, 11.2-11.7 mM, 11.3-11.8 mM, 11.4-11.9 mM, 11.5-12 mM, 11.6-12.1 mM, 11.7-12.2 mM, 11.8-12.3 mM, 11.9-12.4 mM, 12-12.5 mM, 12.1-12.6 mM, 12.2-12.7 mM, 12.3-12.8 mM, 12.4-12.9 mM, 12.5-13 mM, 12.6-13.1 mM, 12.7-13.2 mM, 12.8-13.3 mM, 12.9-13.4 mM, 13-13.5 mM, 13.1-13.6 mM, 13.2-13.7 mM, 13.3-13.8 mM, 13.4-13.9 mM, 13.5-14 mM, 13.6-14.1 mM, 13.7-14.2 mM, 13.8-14.3 mM, 13.9-14.4 mM, 14-14.5 mM, 14.1-14.6 mM, 14.2-14.7 mM, 14.3-14.8 mM, 14.4-14.9 mM, 14.5-15 mM, 0-1 mM, 1-2 mM, 2-3 mM, 3-4 mM, 4-5 mM, 5-6 mM, 6-7 mM, 7-8 mM, 8-9 mM, 9-10 mM, 10-11 mM, 11-12 mM, 12-13 mM, 13-14 mM, 14-15 mM, 15-16 mM, 0-2 mM, 1-3 mM, 2-4 mM, 3-5 mM, 4-6 mM, 5-7 mM, 6-8 mM, 7-9 mM, 8-10 mM, 9-11 mM, 10-12 mM, 11-13 mM, 12-14 mM, 13-15 mM, 0-3 mM, 1-4 mM, 2-5 mM, 3-6 mM, 4-7 mM, 5-8 mM, 6-9 mM, 7-10 mM, 8-11 mM, 9-12 mM, 10-13 mM, 11-14 mM, 12-15 mM, 0-4 mM, 1-5 mM, 2-6 mM, 3-7 mM, 4-8 mM, 5-9 mM, 6-10 mM, 7-11 mM, 8-12 mM, 9-13 mM, 10-14 mM, 11-15 mM, 0-5 mM, 1-6 mM, 2-7 mM, 3-8 mM, 4-9 mM, 5-10 mM, 6-11 mM, 7-12 mM, 8-13 mM, 9-14 mM, 10-15 mM, 0-6 mM, 1-7 mM, 2-8 mM, 3-9 mM, 4-10 mM, 5-11 mM, 6-12 mM, 7-13 mM, 8-14 mM, 9-15 mM, 0-7 mM, 1-8 mM, 2-9 mM, 3-10 mM, 4-11 mM, 5-12 mM, 6-13 mM, 7-14 mM, 8-15 mM, 0-8 mM, 1-9 mM, 2-10 mM, 3-11 mM, 4-12 mM, 5-13 mM, 6-14 mM, 7-15 mM, 0-9 mM, 1-10 mM, 2-11 mM, 3-12 mM, 4-13 mM, 5-14 mM, 6-15 mM, 0-10 mM, 1-11 mM, 2-12 mM, 3-13 mM, 4-14 mM, 5-15 mM, 0-11 mM, 1-12 mM, 2-13 mM, 3-14 mM, 4-15 mM, 0-12 mM, 1-13 mM, 2-14 mM, 3-15 mM, 0-13 mM, 1-14 mM, 2-15 mM, 0-14 mM, 1-15 mM, or 0-15 mM.

In certain embodiments, the formulation may include 0-10 mM of potassium phosphate.

In certain embodiments, the formulation may include 1-3 mM of potassium phosphate.

In certain embodiments, the formulation may include 1-2 mM of potassium phosphate.

In certain embodiments, the formulation may include 2-3 mM of potassium phosphate.

In certain embodiments, the formulation may include 2-12 mM of potassium phosphate.

In certain embodiments, the formulation may include 1.5 mM of potassium phosphate. As a non-limiting example, the formulation may include 1.54 mM of potassium phosphate.

In certain embodiments, the formulation may include 2 mM of potassium phosphate.

### Sodium Chloride

In certain embodiments, at least one of the components in the formulation is sodium chloride.

In certain embodiments, the concentration of sodium chloride in a formulation may be, but is not limited to, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, 100 mM, 101 mM, 102 mM, 103 mM, 104 mM, 105 mM, 106 mM, 107 mM, 108 mM, 109 mM, 110 mM, 111 mM, 112 mM, 113 mM, 114 mM, 115 mM, 116 mM, 117 mM, 118 mM, 119 mM, 120 mM, 121 mM, 122 mM, 123 mM, 124 mM, 125 mM, 126 mM, 127 mM, 128 mM, 129 mM, 130 mM, 131 mM, 132 mM, 133 mM, 134 mM, 135 mM, 136 mM, 137 mM, 138 mM, 139 mM, 140 mM, 141 mM, 142 mM, 143 mM, 144 mM, 145 mM, 146 mM, 147 mM, 148 mM, 149 mM, 150 mM, 151 mM, 152 mM, 153 mM, 154 mM, 155 mM, 156 mM, 157 mM, 158 mM, 159 mM, 160 mM, 161 mM, 162 mM, 163 mM, 164 mM, 165 mM, 166 mM, 167 mM, 168 mM, 169 mM, 170 mM, 171 mM, 172 mM, 173 mM, 174 mM, 175 mM, 176 mM, 177 mM, 178 mM, 179 mM, 180 mM, 181 mM, 182 mM, 183 mM, 184 mM, 185 mM, 186 mM, 187 mM, 188 mM, 189 mM, 190 mM, 191 mM, 192 mM, 193 mM, 194 mM, 195 mM, 196 mM, 197 mM, 198 mM, 199 mM, 200 mM, 201 mM, 202 mM, 203 mM, 204 mM, 205 mM, 206 mM, 207 mM, 208 mM, 209 mM, 210 mM, 211 mM, 212 mM, 213 mM, 214 mM, 215 mM, 216 mM, 217 mM, 218 mM, 219 mM, or 220 mM.

The formulation may include sodium chloride in a range of 75-85 mM, 80-90 mM, 85-95 mM, 90-100 mM, 95-105 mM, 100-110 mM, 105-115 mM, 110-120 mM, 115-125 mM, 120-130 mM 125-135 mM 130-140 mM, 135-145 mM, 140-150 mM, 145-155 mM, 150-160 mM, 155-165 mM, 160-170 mM, 165-175 mM, 170-180 mM, 175-185 mM, 180-190 mM, 185-195 mM, 190-200 mM, 75-95 mM, 80-100 mM, 85-105 mM, 90-110 mM, 95-115 mM, 100-120 mM, 105-125 mM, 110-130 mM, 115-135 mM, 120-140 mM, 125-145 mM, 130-150 mM, 135-155 mM, 140-160 mM, 145-165 mM, 150-170 mM, 155-175 mM, 160-180 mM, 165-185 mM, 170-190 mM, 175-195 mM, 180-200 mM, 75-100 mM, 80-105 mM, 85-110 mM, 90-115 mM, 95-120 mM, 100-125 mM, 105-130 mM, 110-135 mM, 115-140 mM, 120-145 mM, 125-150 mM, 130-155 mM, 135-160 mM, 140-165 mM, 145-170 mM, 150-175 mM, 155-180 mM, 160-185 mM, 165-190 mM, 170-195 mM, 175-200 mM, 75-105 mM, 80-110 mM, 85-115 mM, 90-120 mM, 95-125 mM, 100-130 mM, 105-135 mM, 110-140 mM, 115-145 mM, 120-150 mM, 125-155 mM, 130-160 mM, 135-165 mM, 140-170 mM, 145-175 mM, 150-180 mM, 155-185 mM, 160-190 mM, 165-195 mM, 170-200 mM, 75-115 mM, 80-120 mM, 85-125 mM, 90-130 mM, 95-135 mM, 100-140 mM, 105-145 mM, 110-150 mM, 115-155 mM, 120-160 mM, 125-165 mM, 130-170 mM, 135-175 mM, 140-180 mM, 145-185 mM, 150-190 mM, 155-195 mM, 160-200 mM, 75-120 mM, 80-125 mM, 85-130 mM, 90-135 mM, 95-140 mM, 100-145 mM, 105-150 mM, 110-155 mM, 115-160 mM, 120-165 mM, 125-170 mM, 130-175 mM, 135-180 mM, 140-185 mM, 145-190 mM, 150-195 mM, 155-200 mM, 75-125 mM, 80-130 mM, 85-135 mM, 90-140 mM, 95-145 mM, 100-150 mM, 105-155 mM, 110-160 mM, 115-165 mM, 120-170 mM, 125-175 mM, 130-180 mM, 135-185 mM, 140-190 mM, 145-195 mM, 150-200 mM, 75-125 mM, 80-130 mM, 85-135 mM, 90-140 mM, 95-145 mM, 100-150 mM, 105-155 mM, 110-160 mM, 115-165 mM, 120-170 mM, 125-175 mM, 130-180 mM, 135-185 mM, 140-190 mM, 145-195 mM, 150-200 mM, 75-135 mM, 80-140 mM, 85-145 mM, 90-150 mM, 95-155 mM, 100-160 mM, 105-165 mM, 110-170 mM, 115-175 mM, 120-180 mM, 125-185 mM, 130-190 mM, 135-195 mM, 140-200 mM, 75-145 mM, 80-150 mM, 85-155 mM, 90-160 mM, 95-165 mM, 100-170 mM, 105-175 mM, 110-180 mM, 115-185 mM, 120-190 mM, 125-195 mM, 130-200 mM, 75-155 mM, 80-160 mM, 85-165 mM, 90-170 mM, 95-175 mM, 100-180 mM 105-185 mM, 110-190 mM, 115-195 mM, 120-200 mM, 75-165 mM, 80-170 mM, 85-175 mM, 90-180 mM, 95-185 mM, 100-190 mM, 105-195 mM, 110-200 mM, 75-175 mM, 80-180 mM, 85-185 mM, 90-190 mM, 95-195 mM, 100-200 mM, 80-220 mM, 90-220 mM, 100-220 mM, 110-220 mM, 120-220 mM, 130-220 mM, 140-220 mM, 150-220 mM, 160-220 mM, 170-220 mM, 180-220 mM, 190-220 mM, 200-220 mM, or 210-220 mM.

In certain embodiments, the formulation may include 80-220 mM of sodium chloride.

In certain embodiments, the formulation may include 80-150 mM of sodium chloride.

In certain embodiments, the formulation may include 75 mM of sodium chloride.

In certain embodiments, the formulation may include 83 mM of sodium chloride.

In certain embodiments, the formulation may include 92 mM of sodium chloride.

In certain embodiments, the formulation may include 95 mM of sodium chloride.

In certain embodiments, the formulation may include 98 mM of sodium chloride.

In certain embodiments, the formulation may include 100 mM of sodium chloride.

In certain embodiments, the formulation may include 107 mM of sodium chloride.

In certain embodiments, the formulation may include 109 mM of sodium chloride.

In certain embodiments, the formulation may include 118 mM of sodium chloride.

In certain embodiments, the formulation may include 125 mM of sodium chloride.

In certain embodiments, the formulation may include 127 mM of sodium chloride.

In certain embodiments, the formulation may include 133 mM of sodium chloride.

In certain embodiments, the formulation may include 142 mM of sodium chloride.

In certain embodiments, the formulation may include 150 mM of sodium chloride.

In certain embodiments, the formulation may include 155 mM of sodium chloride.

In certain embodiments, the formulation may include 192 mM of sodium chloride.

In certain embodiments, the formulation may include 210 mM of sodium chloride.

### Potassium Chloride

In certain embodiments, at least one of the components in the formulation is potassium chloride.

In certain embodiments, the concentration of potassium chloride in a formulation may be, but is not limited to, 0.1 mM, 0.2 mM, 0.3 mM, 0.4 mM, 0.5 mM, 0.6 mM, 0.7 mM, 0.8 mM, 0.9 mM, 1 mM, 1.1 mM, 1.2 mM, 1.3 mM, 1.4 mM, 1.5 mM, 1.6 mM, 1.7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.1 mM, 4.2 mM, 4.3 mM, 4.4 mM, 4.5 mM, 4.6 mM, 4.7 mM, 4.8 mM, 4.9 mM, 5 mM, 5.1 mM, 5.2 mM, 5.3 mM, 5.4 mM, 5.5 mM, 5.6 mM, 5.7 mM, 5.8 mM, 5.9 mM, 6 mM, 6.1 mM, 6.2 mM, 6.3 mM, 6.4 mM, 6.5 mM, 6.6 mM, 6.7 mM, 6.8 mM, 6.9 mM, 7 mM, 7.1 mM, 7.2 mM, 7.3 mM, 7.4 mM, 7.5 mM, 7.6 mM, 7.7 mM, 7.8 mM, 7.9 mM, 8 mM, 8.1 mM, 8.2 mM, 8.3 mM, 8.4 mM, 8.5 mM, 8.6 mM, 8.7 mM, 8.8 mM, 8.9 mM, 9 mM, 9.1 mM, 9.2 mM, 9.3 mM, 9.4 mM, 9.5 mM, 9.6 mM, 9.7 mM, 9.8 mM, 9.9 mM, 10 mM, 10.1 mM, 10.2 mM, 10.3 mM, 10.4 mM, 10.5 mM, 10.6 mM, 10.7 mM, 10.8 mM, 10.9 mM, 11 mM, 11.1 mM, 11.2 mM, 11.3 mM, 11.4 mM, 11.5 mM, 11.6 mM, 11.7 mM, 11.8 mM, 11.9 mM, 12 mM, 12.1 mM, 12.2 mM, 12.3 mM, 12.4 mM, 12.5 mM, 12.6 mM, 12.7 mM, 12.8 mM, 12.9 mM, 13 mM, 13.1 mM, 13.2 mM, 13.3 mM, 13.4 mM, 13.5 mM, 13.6 mM, 13.7 mM, 13.8 mM, 13.9 mM, 14 mM, 14.1 mM, 14.2 mM, 14.3 mM, 14.4 mM, 14.5 mM, 14.6 mM, 14.7 mM, 14.8 mM, 14.9 mM or 15 mM.

The formulation may include potassium chloride in a range of 0-0.5 mM, 0.1-0.6 mM, 0.2-0.7 mM, 0.3-0.8 mM, 0.4-0.9 mM, 0.5-1 mM, 0.6-1.1 mM, 0.7-1.2 mM, 0.8-1.3 mM, 0.9-1.4 mM, 1-1.5 mM, 1.1-1.6 mM, 1.2-1.7 mM, 1.3-1.8 mM, 1.4-1.9 mM, 1.5-2 mM, 1.6-2.1 mM, 1.7-2.2 mM, 1.8-2.3 mM, 1.9-2.4 mM, 2-2.5 mM, 2.1-2.6 mM, 2.2-2.7 mM, 2.3-2.8 mM, 2.4-2.9 mM, 2.5-3 mM, 2.6-3.1 mM, 2.7-3.2 mM, 2.8-3.3 mM, 2.9-3.4 mM, 3-3.5 mM, 3.1-3.6 mM, 3.2-3.7 mM, 3.3-3.8 mM, 3.4-3.9 mM, 3.5-4 mM, 3.6-4.1 mM, 3.7-4.2 mM, 3.8-4.3 mM, 3.9-4.4 mM, 4-4.5 mM, 4.1-4.6 mM, 4.2-4.7 mM, 4.3-4.8 mM, 4.4-4.9 mM, 4.5-5 mM, 4.6-5.1 mM, 4.7-5.2 mM, 4.8-5.3 mM, 4.9-5.4 mM, 5-5.5 mM, 5.1-5.6 mM, 5.2-5.7 mM, 5.3-5.8 mM, 5.4-5.9 mM, 5.5-6 mM, 5.6-6.1 mM, 5.7-6.2 mM, 5.8-6.3 mM, 5.9-6.4 mM, 6-6.5 mM, 6.1-6.6 mM, 6.2-6.7 mM, 6.3-6.8 mM, 6.4-6.9 mM, 6.5-7 mM, 6.6-7.1 mM, 6.7-7.2 mM, 6.8-7.3 mM, 6.9-7.4 mM, 7-7.5 mM, 7.1-7.6 mM, 7.2-7.7 mM, 7.3-7.8 mM, 7.4-7.9 mM, 7.5-8 mM, 7.6-8.1 mM, 7.7-8.2 mM, 7.8-8.3 mM, 7.9-8.4 mM, 8-8.5 mM, 8.1-8.6 mM, 8.2-8.7 mM, 8.3-8.8 mM, 8.4-8.9 mM, 8.5-9 mM, 8.6-9.1 mM, 8.7-9.2 mM, 8.8-9.3 mM, 8.9-9.4 mM, 9-9.5 mM, 9.1-9.6 mM, 9.2-9.7 mM, 9.3-9.8 mM, 9.4-9.9 mM, 9.5-10 mM, 9.6-10.1 mM, 9.7-10.2 mM, 9.8-10.3 mM, 9.9-10.4 mM, 10-10.5 mM, 10.1-10.6 mM, 10.2-10.7 mM, 10.3-10.8 mM, 10.4-10.9 mM, 10.5-11 mM, 10.6-11.1 mM, 10.7-11.2 mM, 10.8-11.3 mM, 10.9-11.4 mM, 11-11.5 mM, 11.1-11.6 mM, 11.2-11.7 mM, 11.3-11.8 mM, 11.4-11.9 mM, 11.5-12 mM, 11.6-12.1 mM, 11.7-12.2 mM, 11.8-12.3 mM, 11.9-12.4 mM, 12-12.5 mM, 12.1-12.6 mM, 12.2-12.7 mM, 12.3-12.8 mM, 12.4-12.9 mM, 12.5-13 mM, 12.6-13.1 mM, 12.7-13.2 mM, 12.8-13.3 mM, 12.9-13.4 mM, 13-13.5 mM, 13.1-13.6 mM, 13.2-13.7 mM, 13.3-13.8 mM, 13.4-13.9 mM, 13.5-14 mM, 13.6-14.1 mM, 13.7-14.2 mM, 13.8-14.3 mM, 13.9-14.4 mM, 14-14.5 mM, 14.1-14.6 mM, 14.2-14.7 mM, 14.3-14.8 mM, 14.4-14.9 mM, 14.5-15 mM, 0-1 mM, 1-2 mM, 2-3 mM, 3-4 mM, 4-5 mM, 5-6 mM, 6-7 mM, 7-8 mM, 8-9 mM, 9-10 mM, 10-11 mM, 11-12 mM, 12-13 mM, 13-14 mM, 14-15 mM, 15-16 mM, 0-2 mM, 1-3 mM, 2-4 mM, 3-5 mM, 4-6 mM, 5-7 mM, 6-8 mM, 7-9 mM, 8-10 mM, 9-11 mM, 10-12 mM, 11-13 mM, 12-14 mM, 13-15 mM, 0-3 mM, 1-4 mM, 2-5 mM, 3-6 mM, 4-7 mM, 5-8 mM, 6-9 mM, 7-10 mM, 8-11 mM, 9-12 mM, 10-13 mM, 11-14 mM, 12-15 mM, 0-4 mM, 1-5 mM, 2-6 mM, 3-7 mM, 4-8 mM, 5-9 mM, 6-10 mM, 7-11 mM, 8-12 mM, 9-13 mM, 10-14 mM, 11-15 mM, 0-5 mM, 1-6 mM, 2-7 mM, 3-8 mM, 4-9 mM, 5-10 mM, 6-11 mM, 7-12 mM, 8-13 mM, 9-14 mM, 10-15 mM, 0-6 mM, 1-7 mM, 2-8 mM, 3-9 mM, 4-10 mM, 5-11 mM, 6-12 mM, 7-13 mM, 8-14 mM, 9-15 mM, 0-7 mM, 1-8 mM, 2-9 mM, 3-10 mM, 4-11 mM, 5-12 mM, 6-13 mM, 7-14 mM, 8-15 mM, 0-8 mM, 1-9 mM, 2-10 mM, 3-11 mM, 4-12 mM, 5-13 mM, 6-14 mM, 7-15 mM, 0-9 mM, 1-10 mM, 2-11 mM, 3-12 mM, 4-13 mM, 5-14 mM, 6-15 mM, 0-10 mM, 1-11 mM, 2-12 mM, 3-13 mM, 4-14 mM, 5-15 mM, 0-11 mM, 1-12 mM, 2-13 mM, 3-14 mM, 4-15 mM, 0-12 mM, 1-13 mM, 2-14 mM, 3-15 mM, 0-13 mM, 1-14 mM, 2-15 mM, 0-14 mM, 1-15 mM, or 0-15 mM.

In certain embodiments, the formulation may include 0-10 mM of potassium chloride.

In certain embodiments, the formulation may include 1-3 mM of potassium chloride.

In certain embodiments, the formulation may include 1-2 mM of potassium chloride.

In certain embodiments, the formulation may include 2-3 mM of potassium chloride.

In certain embodiments, the formulation may include 1.5 mM of potassium chloride.

In certain embodiments, the formulation may include 2.7 mM of potassium chloride.

### Magnesium Chloride

In certain embodiments, at least one of the components in the formulation is magnesium chloride.

In certain embodiments, the concentration of magnesium chloride may be, but is not limited to, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM, 60 mM, 61 mM, 62 mM, 63 mM, 64 mM, 65 mM, 66 mM, 67 mM, 68 mM, 69 mM, 70 mM, 71 mM, 72 mM, 73 mM, 74 mM, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, or 100 mM.

The formulation may include magnesium chloride in a range of 0-5 mM, 1-5 mM, 2-5 mM, 3-5 mM, 4-5 mM, 0-10 mM, 1-10 mM, 2-10 mM, 3-10 mM, 4-10 mM, 5-10 mM, 6-10 mM, 7-10 mM, 8-10 mM, 9-10 mM, 0-25 mM, 1-25 mM, 2-25 mM, 3-25 mM, 4-25 mM, 5-25 mM, 6-25 mM, 7-25 mM, 8-25 mM, 9-25 mM, 10-25 mM, 11-25 mM, 12-25 mM, 13-25 mM, 14-25 mM, 15-25 mM, 16-25 mM, 17-25 mM, 18-25 mM, 19-25 mM, 20-25 mM, 21-25 mM, 22-25 mM, 23-25 mM, 24-25 mM, 0-50 mM, 1-50 mM, 2-50 mM, 3-50 mM, 4-50 mM, 5-50 mM, 6-50 mM, 7-50 mM, 8-50 mM, 9-50 mM, 10-50 mM, 11-50 mM, 12-50 mM, 13-50 mM, 14-50 mM, 15-50 mM, 16-50 mM, 17-50 mM, 18-50 mM, 19-50 mM, 20-50 mM, 21-50 mM, 22-50 mM, 23-50 mM, 24-50 mM, 25-50 mM, 26-50 mM, 27-50 mM, 28-50 mM, 29-50 mM, 30-50 mM, 31-50 mM, 32-50 mM, 33-50 mM, 34-50 mM, 35-50 mM, 36-50 mM, 37-50 mM, 38-50 mM, 39-50 mM, 40-50 mM, 41-50 mM, 42-50 mM, 43-50 mM, 44-50 mM, 45-50 mM, 46-50 mM, 47-50 mM, 48-50 mM, 49-50 mM, 0-75 mM, 1-75 mM, 2-75 mM, 3-75 mM, 4-75 mM, 5-75 mM, 6-75 mM, 7-75 mM, 8-75 mM, 9-75 mM, 10-75 mM, 11-75 mM, 12-75 mM, 13-75 mM, 14-75 mM, 15-75 mM, 16-75 mM, 17-75 mM, 18-75 mM, 19-75 mM, 20-75 mM, 21-75 mM, 22-75 mM, 23-75 mM, 24-75 mM, 25-75 mM, 26-75 mM, 27-75 mM, 28-75 mM, 29-75 mM, 30-75 mM, 31-75 mM, 32-75 mM, 33-75 mM, 34-75 mM, 35-75 mM, 36-75 mM, 37-75 mM, 38-75 mM, 39-75 mM, 40-75 mM, 41-75 mM, 42-75 mM, 43-75 mM, 44-75 mM, 45-75 mM, 46-75 mM, 47-75 mM, 48-75 mM, 49-75 mM, 50-75 mM, 51-75 mM, 52-75 mM, 53-75 mM, 54-75 mM, 55-75 mM, 56-75 mM, 57-75 mM, 58-75 mM, 59-75 mM, 60-75 mM, 61-75 mM, 62-75 mM, 63-75 mM, 64-75 mM, 65-75 mM, 66-75 mM, 67-75 mM, 68-75 mM, 69-75 mM, 70-75 mM, 71-75 mM, 72-75 mM, 73-75 mM, 74-75 mM, 50-100 mM, 60-100 mM, 75-100 mM, 80-100 mM, or 90-100 mM.

In certain embodiments, the formulation may include 0-75 mM of magnesium chloride.

In certain embodiments, the formulation may include 0-5 mM of magnesium chloride.

In certain embodiments, the formulation may include 50-100 mM of magnesium chloride.

In certain embodiments, the formulation may include 2 mM of magnesium chloride.

In certain embodiments, the formulation may include 75 mM of magnesium chloride.

### Tris

In certain embodiments, at least one of the components in the formulation is Tris (also called tris(hydroxymethyl)aminomethane, tromethamine or THAM).

In certain embodiments, the concentration of Tris in a formulation may be, but is not limited to, 0.1 mM, 0.2 mM, 0.3 mM, 0.4 mM, 0.5 mM, 0.6 mM, 0.7 mM, 0.8 mM, 0.9 mM, 1 mM, 1.1 mM, 1.2 mM, 1.3 mM, 1.4 mM, 1.5 mM, 1.6 mM, 1.7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.1 mM, 4.2 mM, 4.3 mM, 4.4 mM, 4.5 mM, 4.6 mM, 4.7 mM, 4.8 mM, 4.9 mM, 5 mM, 5.1 mM, 5.2 mM, 5.3 mM, 5.4 mM, 5.5 mM, 5.6 mM, 5.7 mM, 5.8 mM, 5.9 mM, 6 mM, 6.1 mM, 6.2 mM, 6.3 mM, 6.4 mM, 6.5 mM, 6.6 mM, 6.7 mM, 6.8 mM, 6.9 mM, 7 mM, 7.1 mM, 7.2 mM, 7.3 mM, 7.4 mM, 7.5 mM, 7.6 mM, 7.7 mM, 7.8 mM, 7.9 mM, 8 mM, 8.1 mM, 8.2 mM, 8.3 mM, 8.4 mM, 8.5 mM, 8.6 mM, 8.7 mM, 8.8 mM, 8.9 mM, 9 mM, 9.1 mM, 9.2 mM, 9.3 mM, 9.4 mM, 9.5 mM, 9.6 mM, 9.7 mM, 9.8 mM, 9.9 mM, 10 mM, 10.1 mM, 10.2 mM, 10.3 mM, 10.4 mM, 10.5 mM, 10.6 mM, 10.7 mM, 10.8 mM, 10.9 mM, 11 mM, 11.1 mM, 11.2 mM, 11.3 mM, 11.4 mM, 11.5 mM, 11.6 mM, 11.7 mM, 11.8 mM, 11.9 mM, 12 mM, 12.1 mM, 12.2 mM, 12.3 mM, 12.4 mM, 12.5 mM, 12.6 mM, 12.7 mM, 12.8 mM, 12.9 mM, 13 mM, 13.1 mM, 13.2 mM, 13.3 mM, 13.4 mM, 13.5 mM, 13.6 mM, 13.7 mM, 13.8 mM, 13.9 mM, 14 mM, 14.1 mM, 14.2 mM, 14.3 mM, 14.4 mM, 14.5 mM, 14.6 mM, 14.7 mM, 14.8 mM, 14.9 mM or 15 mM.

The formulation may include Tris in a range of 0-0.5 mM, 0.1-0.6 mM, 0.2-0.7 mM, 0.3-0.8 mM, 0.4-0.9 mM, 0.5-1 mM, 0.6-1.1 mM, 0.7-1.2 mM, 0.8-1.3 mM, 0.9-1.4 mM, 1-1.5 mM, 1.1-1.6 mM, 1.2-1.7 mM, 1.3-1.8 mM, 1.4-1.9 mM, 1.5-2 mM, 1.6-2.1 mM, 1.7-2.2 mM, 1.8-2.3 mM, 1.9-2.4 mM, 2-2.5 mM, 2.1-2.6 mM, 2.2-2.7 mM, 2.3-2.8 mM, 2.4-2.9 mM, 2.5-3 mM, 2.6-3.1 mM, 2.7-3.2 mM, 2.8-3.3 mM, 2.9-3.4 mM, 3-3.5 mM, 3.1-3.6 mM, 3.2-3.7 mM, 3.3-3.8 mM, 3.4-3.9 mM, 3.5-4 mM, 3.6-4.1 mM, 3.7-4.2 mM, 3.8-4.3 mM, 3.9-4.4 mM, 4-4.5 mM, 4.1-4.6 mM, 4.2-4.7 mM, 4.3-4.8 mM, 4.4-4.9 mM, 4.5-5 mM, 4.6-5.1 mM, 4.7-5.2 mM, 4.8-5.3 mM, 4.9-5.4 mM, 5-5.5 mM, 5.1-5.6 mM, 5.2-5.7 mM, 5.3-5.8 mM, 5.4-5.9 mM, 5.5-6 mM, 5.6-6.1 mM, 5.7-6.2 mM, 5.8-6.3 mM, 5.9-6.4 mM, 6-6.5 mM, 6.1-6.6 mM, 6.2-6.7 mM, 6.3-6.8 mM, 6.4-6.9 mM, 6.5-7 mM, 6.6-7.1 mM, 6.7-7.2 mM, 6.8-7.3 mM, 6.9-7.4 mM, 7-7.5 mM, 7.1-7.6 mM, 7.2-7.7 mM, 7.3-7.8 mM, 7.4-7.9 mM, 7.5-8 mM, 7.6-8.1 mM, 7.7-8.2 mM, 7.8-8.3 mM, 7.9-8.4 mM, 8-8.5 mM, 8.1-8.6 mM, 8.2-8.7 mM, 8.3-8.8 mM, 8.4-8.9 mM, 8.5-9 mM, 8.6-9.1 mM, 8.7-9.2 mM, 8.8-9.3 mM, 8.9-9.4 mM, 9-9.5 mM, 9.1-9.6 mM, 9.2-9.7 mM, 9.3-9.8 mM, 9.4-9.9 mM, 9.5-10 mM, 9.6-10.1 mM, 9.7-10.2 mM, 9.8-10.3 mM, 9.9-10.4 mM, 10-10.5 mM, 10.1-10.6 mM, 10.2-10.7 mM, 10.3-10.8 mM, 10.4-10.9 mM, 10.5-11 mM, 10.6-11.1 mM, 10.7-11.2 mM, 10.8-11.3 mM, 10.9-11.4 mM, 11-11.5 mM, 11.1-11.6 mM, 11.2-11.7 mM, 11.3-11.8 mM, 11.4-11.9 mM, 11.5-12 mM, 11.6-12.1 mM, 11.7-12.2 mM, 11.8-12.3 mM, 11.9-12.4 mM, 12-12.5 mM, 12.1-12.6 mM 12.2-12.7 mM, 12.3-12.8 mM, 12.4-12.9 mM, 12.5-13 mM, 12.6-13.1 mM, 12.7-13.2 mM, 12.8-13.3 mM, 12.9-13.4 mM, 13-13.5 mM, 13.1-13.6 mM, 13.2-13.7 mM, 13.3-13.8 mM, 13.4-13.9 mM, 13.5-14 mM, 13.6-14.1 mM, 13.7-14.2 mM, 13.8-14.3 mM, 13.9-14.4 mM, 14-14.5 mM, 14.1-14.6 mM, 14.2-14.7 mM, 14.3-14.8 mM, 14.4-14.9 mM, 14.5-15 mM, 0-1 mM, 1-2 mM, 2-3 mM, 3-4 mM, 4-5 mM, 5-6 mM, 6-7 mM, 7-8 mM, 8-9 mM, 9-10 mM, 10-11 mM, 11-12 mM, 12-13 mM, 13-14 mM, 14-15 mM, 15-16 mM, 0-2 mM, 1-3 mM, 2-4 mM, 3-5 mM, 4-6 mM, 5-7 mM, 6-8 mM, 7-9 mM, 8-10 mM, 9-11 mM, 10-12 mM, 11-13 mM, 12-14 mM, 13-15 mM, 0-3 mM, 1-4 mM, 2-5 mM, 3-6 mM, 4-7 mM, 5-8 mM, 6-9 mM, 7-10 mM, 8-11 mM, 9-12 mM, 10-13 mM, 11-14 mM, 12-15 mM, 0-4 mM, 1-5 mM, 2-6 mM, 3-7 mM, 4-8 mM, 5-9 mM, 6-10 mM, 7-11 mM, 8-12 mM, 9-13 mM, 10-14 mM, 11-15 mM, 0-5 mM, 1-6 mM, 2-7 mM, 3-8 mM, 4-9 mM, 5-10 mM, 6-11 mM, 7-12 mM, 8-13 mM, 9-14 mM, 10-15 mM, 0-6 mM, 1-7 mM, 2-8 mM, 3-9 mM, 4-10 mM, 5-11 mM, 6-12 mM, 7-13 mM, 8-14 mM, 9-15 mM, 0-7 mM, 1-8 mM, 2-9 mM, 3-10 mM, 4-11 mM, 5-12 mM, 6-13 mM, 7-14 mM, 8-15 mM, 0-8 mM, 1-9 mM, 2-10 mM, 3-11 mM, 4-12 mM, 5-13 mM, 6-14 mM, 7-15 mM, 0-9 mM, 1-10 mM, 2-11 mM, 3-12 mM, 4-13 mM, 5-14 mM, 6-15 mM, 0-10 mM, 1-11 mM, 2-12 mM, 3-13 mM, 4-14 mM, 5-15 mM, 0-11 mM, 1-12 mM, 2-13 mM, 3-14 mM, 4-15 mM, 0-12 mM, 1-13 mM, 2-14 mM, 3-15 mM, 0-13 mM, 1-14 mM, 2-15 mM, 0-14 mM, 1-15 mM, or 0-15 mM.

In certain embodiments, the formulation may include 0-10 mM of Tris.

In certain embodiments, the formulation may include 2-12 mM of Tris.

In certain embodiments, the formulation may include 10 mM of Tris.

### Histidine

In certain embodiments, at least one of the components in the formulation is Histidine.

In certain embodiments, the concentration of Histidine in a formulation may be, but is not limited to, 0.1 mM, 0.2 mM, 0.3 mM, 0.4 mM, 0.5 mM, 0.6 mM, 0.7 mM, 0.8 mM, 0.9 mM, 1 mM, 1.1 mM, 1.2 mM, 1.3 mM, 1.4 mM, 1.5 mM, 1.6 mM, 1.7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.1 mM, 4.2 mM, 4.3 mM, 4.4 mM, 4.5 mM, 4.6 mM, 4.7 mM, 4.8 mM, 4.9 mM, 5 mM, 5.1 mM, 5.2 mM, 5.3 mM, 5.4 mM, 5.5 mM, 5.6 mM, 5.7 mM, 5.8 mM, 5.9 mM, 6 mM, 6.1 mM, 6.2 mM, 6.3 mM, 6.4 mM, 6.5 mM, 6.6 mM, 6.7 mM, 6.8 mM, 6.9 mM, 7 mM, 7.1 mM, 7.2 mM, 7.3 mM, 7.4 mM, 7.5 mM, 7.6 mM, 7.7 mM, 7.8 mM, 7.9 mM, 8 mM, 8.1 mM, 8.2 mM, 8.3 mM, 8.4 mM, 8.5 mM, 8.6 mM, 8.7 mM, 8.8 mM, 8.9 mM, 9 mM, 9.1 mM, 9.2 mM, 9.3 mM, 9.4 mM, 9.5 mM, 9.6 mM, 9.7 mM, 9.8 mM, 9.9 mM, 10 mM, 10.1 mM, 10.2 mM, 10.3 mM, 10.4 mM, 10.5 mM, 10.6 mM, 10.7 mM, 10.8 mM, 10.9 mM, 11 mM, 11.1 mM, 11.2 mM, 11.3 mM, 11.4 mM, 11.5 mM, 11.6 mM, 11.7 mM, 11.8 mM, 11.9 mM, 12 mM, 12.1 mM, 12.2 mM, 12.3 mM, 12.4 mM, 12.5 mM, 12.6 mM, 12.7 mM, 12.8 mM, 12.9 mM, 13 mM, 13.1 mM, 13.2 mM, 13.3 mM, 13.4 mM, 13.5 mM, 13.6 mM, 13.7 mM, 13.8 mM, 13.9 mM, 14 mM, 14.1 mM, 14.2 mM, 14.3 mM, 14.4 mM, 14.5 mM, 14.6 mM, 14.7 mM, 14.8 mM, 14.9 mM or 15 mM.

The formulation may include Histidine in a range of 0-0.5 mM, 0.1-0.6 mM, 0.2-0.7 mM, 0.3-0.8 mM, 0.4-0.9 mM, 0.5-1 mM, 0.6-1.1 mM, 0.7-1.2 mM, 0.8-1.3 mM, 0.9-1.4 mM, 1-1.5 mM, 1.1-1.6 mM, 1.2-1.7 mM, 1.3-1.8 mM, 1.4-1.9 mM, 1.5-2 mM, 1.6-2.1 mM, 1.7-2.2 mM, 1.8-2.3 mM, 1.9-2.4 mM, 2-2.5 mM, 2.1-2.6 mM, 2.2-2.7 mM, 2.3-2.8 mM, 2.4-2.9 mM, 2.5-3 mM, 2.6-3.1 mM, 2.7-3.2 mM, 2.8-3.3 mM, 2.9-3.4 mM, 3-3.5 mM, 3.1-3.6 mM, 3.2-3.7 mM, 3.3-3.8 mM, 3.4-3.9 mM, 3.5-4 mM, 3.6-4.1 mM, 3.7-4.2 mM, 3.8-4.3 mM, 3.9-4.4 mM, 4-4.5 mM, 4.1-4.6 mM, 4.2-4.7 mM, 4.3-4.8 mM, 4.4-4.9 mM, 4.5-5 mM, 4.6-5.1 mM, 4.7-5.2 mM, 4.8-5.3 mM, 4.9-5.4 mM, 5-5.5 mM, 5.1-5.6 mM, 5.2-5.7 mM, 5.3-5.8 mM, 5.4-5.9 mM, 5.5-6 mM, 5.6-6.1 mM, 5.7-6.2 mM, 5.8-6.3 mM, 5.9-6.4 mM, 6-6.5 mM, 6.1-6.6 mM, 6.2-6.7 mM, 6.3-6.8 mM, 6.4-6.9 mM, 6.5-7 mM, 6.6-7.1 mM, 6.7-7.2 mM, 6.8-7.3 mM, 6.9-7.4 mM, 7-7.5 mM, 7.1-7.6 mM, 7.2-7.7 mM, 7.3-7.8 mM, 7.4-7.9 mM, 7.5-8 mM, 7.6-8.1 mM, 7.7-8.2 mM, 7.8-8.3 mM, 7.9-8.4 mM, 8-8.5 mM, 8.1-8.6 mM, 8.2-8.7 mM, 8.3-8.8 mM, 8.4-8.9 mM, 8.5-9 mM, 8.6-9.1 mM, 8.7-9.2 mM, 8.8-9.3 mM, 8.9-9.4 mM, 9-9.5 mM, 9.1-9.6 mM, 9.2-9.7 mM, 9.3-9.8 mM, 9.4-9.9 mM, 9.5-10 mM, 9.6-10.1 mM, 9.7-10.2 mM, 9.8-10.3 mM, 9.9-10.4 mM, 10-10.5 mM, 10.1-10.6 mM, 10.2-10.7 mM, 10.3-10.8 mM, 10.4-10.9 mM, 10.5-11 mM, 10.6-11.1 mM, 10.7-11.2 mM, 10.8-11.3 mM, 10.9-11.4 mM, 11-11.5 mM, 11.1-11.6 mM, 11.2-11.7 mM, 11.3-11.8 mM, 11.4-11.9 mM, 11.5-12 mM, 11.6-12.1 mM, 11.7-12.2 mM, 11.8-12.3 mM, 11.9-12.4 mM, 12-12.5 mM, 12.1-12.6 mM, 12.2-12.7 mM, 12.3-12.8 mM, 12.4-12.9 mM, 12.5-13 mM, 12.6-13.1 mM, 12.7-13.2 mM, 12.8-13.3 mM, 12.9-13.4 mM, 13-13.5 mM, 13.1-13.6 mM, 13.2-13.7 mM, 13.3-13.8 mM, 13.4-13.9 mM, 13.5-14 mM, 13.6-14.1 mM, 13.7-14.2 mM, 13.8-14.3 mM, 13.9-14.4 mM 14-14.5 mM, 14.1-14.6 mM, 14.2-14.7 mM, 14.3-14.8 mM, 14.4-14.9 mM, 14.5-15 mM, 0-1 mM, 1-2 mM, 2-3 mM, 3-4 mM, 4-5 mM, 5-6 mM, 6-7 mM, 7-8 mM, 8-9 mM, 9-10 mM, 10-11 mM, 11-12 mM, 12-13 mM, 13-14 mM, 14-15 mM, 15-16 mM, 0-2 mM, 1-3 mM, 2-4 mM, 3-5 mM, 4-6 mM, 5-7 mM, 6-8 mM, 7-9 mM, 8-10 mM, 9-11 mM, 10-12 mM, 11-13 mM, 12-14 mM, 13-15 mM, 0-3 mM, 1-4 mM, 2-5 mM, 3-6 mM, 4-7 mM, 5-8 mM, 6-9 mM, 7-10 mM, 8-11 mM, 9-12 mM, 10-13 mM, 11-14 mM, 12-15 mM, 0-4 mM, 1-5 mM, 2-6 mM, 3-7 mM, 4-8 mM, 5-9 mM, 6-10 mM, 7-11 mM, 8-12 mM, 9-13 mM, 10-14 mM, 11-15 mM, 0-5 mM, 1-6 mM, 2-7 mM, 3-8 mM, 4-9 mM, 5-10 mM, 6-11 mM, 7-12 mM, 8-13 mM, 9-14 mM, 10-15 mM, 0-6 mM, 1-7 mM, 2-8 mM, 3-9 mM, 4-10 mM, 5-11 mM, 6-12 mM, 7-13 mM, 8-14 mM, 9-15 mM, 0-7 mM, 1-8 mM, 2-9 mM, 3-10 mM, 4-11 mM, 5-12 mM, 6-13 mM, 7-14 mM, 8-15 mM, 0-8 mM, 1-9 mM, 2-10 mM, 3-11 mM, 4-12 mM, 5-13 mM, 6-14 mM, 7-15 mM, 0-9 mM, 1-10 mM, 2-11 mM, 3-12 mM, 4-13 mM, 5-14 mM, 6-15 mM, 0-10 mM, 1-11 mM, 2-12 mM, 3-13 mM, 4-14 mM, 5-15 mM, 0-11 mM, 1-12 mM, 2-13 mM, 3-14 mM, 4-15 mM, 0-12 mM, 1-13 mM, 2-14 mM, 3-15 mM, 0-13 mM, 1-14 mM, 2-15 mM, 0-14 mM, 1-15 mM, or 0-15 mM.

In certain embodiments, the formulation may include 0-10 mM of Histidine.

In certain embodiments, the formulation may include 2-12 mM of Histidine.

In certain embodiments, the formulation may include 10 mM of Histidine.

### Arginine

In certain embodiments, at least one of the components in the formulation is arginine.

In certain embodiments, the concentration of arginine may be, but is not limited to, 1 mM, 2 mM, 3 mM, 4 mM, 5 mM, 6 mM, 7 mM, 8 mM, 9 mM, 10 mM, 11 mM, 12 mM 13 mM, 14 mM, 15 mM, 16 mM, 17 mM, 18 mM, 19 mM 20 mM, 21 mM 22 mM, 23 mM 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM 52 mM, 53 mM 54 mM, 55 mM 56 mM, 57 mM, 58 mM, 59 mM, 60 mM, 61 mM, 62 mM, 63 mM, 64 mM, 65 mM, 66 mM, 67 mM, 68 mM, 69 mM, 70 mM, 71 mM, 72 mM, 73 mM, 74 mM, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, or 100 mM.

The formulation may include arginine in a range of 0-5 mM, 1-5 mM, 2-5 mM, 3-5 mM, 4-5 mM, 0-10 mM, 1-10 mM, 2-10 mM, 3-10 mM, 4-10 mM, 5-10 mM, 6-10 mM, 7-10 mM, 8-10 mM, 9-10 mM, 0-25 mM, 1-25 mM, 2-25 mM, 3-25 mM, 4-25 mM, 5-25 mM, 6-25 mM, 7-25 mM, 8-25 mM, 9-25 mM, 10-25 mM, 11-25 mM, 12-25 mM, 13-25 mM, 14-25 mM, 15-25 mM, 16-25 mM, 17-25 mM, 18-25 mM, 19-25 mM, 20-25 mM, 21-25 mM, 22-25 mM, 23-25 mM, 24-25 mM, 0-50 mM, 1-50 mM, 2-50 mM, 3-50 mM, 4-50 mM, 5-50 mM, 6-50 mM, 7-50 mM, 8-50 mM, 9-50 mM, 10-50 mM, 11-50 mM, 12-50 mM, 13-50 mM, 14-50 mM, 15-50 mM, 16-50 mM, 17-50 mM, 18-50 mM, 19-50 mM, 20-50 mM, 21-50 mM, 22-50 mM, 23-50 mM, 24-50 mM, 25-50 mM, 26-50 mM, 27-50 mM, 28-50 mM, 29-50 mM, 30-50 mM, 31-50 mM, 32-50 mM, 33-50 mM, 34-50 mM, 35-50 mM, 36-50 mM, 37-50 mM, 38-50 mM, 39-50 mM, 40-50 mM, 41-50 mM, 42-50 mM, 43-50 mM, 44-50 mM, 45-50 mM, 46-50 mM, 47-50 mM, 48-50 mM, 49-50 mM, 0-75 mM, 1-75 mM, 2-75 mM, 3-75 mM, 4-75 mM, 5-75 mM, 6-75 mM, 7-75 mM, 8-75 mM, 9-75 mM, 10-75 mM, 11-75 mM, 12-75 mM, 13-75 mM, 14-75 mM, 15-75 mM, 16-75 mM, 17-75 mM, 18-75 mM, 19-75 mM, 20-75 mM, 21-75 mM, 22-75 mM, 23-75 mM, 24-75 mM, 25-75 mM, 26-75 mM, 27-75 mM, 28-75 mM, 29-75 mM, 30-75 mM, 31-75 mM, 32-75 mM, 33-75 mM, 34-75 mM, 35-75 mM, 36-75 mM, 37-75 mM, 38-75 mM, 39-75 mM, 40-75 mM, 41-75 mM, 42-75 mM, 43-75 mM, 44-75 mM, 45-75 mM, 46-75 mM, 47-75 mM, 48-75 mM, 49-75 mM, 50-75 mM, 51-75 mM, 52-75 mM, 53-75 mM, 54-75 mM, 55-75 mM, 56-75 mM, 57-75 mM, 58-75 mM, 59-75 mM, 60-75 mM, 61-75 mM, 62-75 mM, 63-75 mM, 64-75 mM, 65-75 mM, 66-75 mM, 67-75 mM, 68-75 mM, 69-75 mM, 70-75 mM, 71-75 mM, 72-75 mM, 73-75 mM, 74-75 mM, 50-100 mM, 60-100 mM, 75-100 mM, 80-100 mM, or 90-100 mM.

In certain embodiments, the formulation may include 0-75 mM of arginine.

In certain embodiments, the formulation may include 50-100 mM of arginine.

In certain embodiments, the formulation may include 75 mM of arginine.

### Hydrochloric Acid

In certain embodiments, at least one of the components in the formulation is hydrochloric acid.

In certain embodiments, the concentration of hydrochloric acid in a formulation may be, but is not limited to, 0.1 mM, 0.2 mM, 0.3 mM, 0.4 mM, 0.5 mM, 0.6 mM, 0.7 mM, 0.8 mM, 0.9 mM, 1 mM, 1.1 mM, 1.2 mM, 1.3 mM, 1.4 mM, 1.5 mM, 1.6 mM, 1.7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.1 mM, 4.2 mM, 4.3 mM, 4.4 mM, 4.5 mM, 4.6 mM, 4.7 mM, 4.8 mM, 4.9 mM, 5 mM, 5.1 mM, 5.2 mM, 5.3 mM, 5.4 mM, 5.5 mM, 5.6 mM, 5.7 mM, 5.8 mM, 5.9 mM, 6 mM, 6.1 mM, 6.2 mM, 6.3 mM, 6.4 mM, 6.5 mM, 6.6 mM, 6.7 mM, 6.8 mM, 6.9 mM, 7 mM, 7.1 mM, 7.2 mM, 7.3 mM, 7.4 mM, 7.5 mM, 7.6 mM, 7.7 mM, 7.8 mM, 7.9 mM, 8 mM, 8.1 mM, 8.2 mM, 8.3 mM, 8.4 mM, 8.5 mM, 8.6 mM, 8.7 mM, 8.8 mM, 8.9 mM, 9 mM, 9.1 mM, 9.2 mM, 9.3 mM, 9.4 mM, 9.5 mM, 9.6 mM, 9.7 mM, 9.8 mM, 9.9 mM, 10 mM, 10.1 mM, 10.2 mM, 10.3 mM, 10.4 mM, 10.5 mM, 10.6 mM, 10.7 mM, 10.8 mM, 10.9 mM, 11 mM, 11.1 mM, 11.2 mM, 11.3 mM, 11.4 mM, 11.5 mM, 11.6 mM, 11.7 mM, 11.8 mM, 11.9 mM, 12 mM, 12.1 mM, 12.2 mM, 12.3 mM, 12.4 mM, 12.5 mM, 12.6 mM, 12.7 mM, 12.8 mM, 12.9 mM, 13 mM, 13.1 mM, 13.2 mM, 13.3 mM, 13.4 mM, 13.5 mM, 13.6 mM, 13.7 mM, 13.8 mM, 13.9 mM, 14 mM, 14.1 mM, 14.2 mM, 14.3 mM, 14.4 mM, 14.5 mM, 14.6 mM, 14.7 mM, 14.8 mM, 14.9 mM or 15 mM.

The formulation may include hydrochloric acid in a range of 0-0.5 mM, 0.1-0.6 mM, 0.2-0.7 mM, 0.3-0.8 mM, 0.4-0.9 mM, 0.5-1 mM, 0.6-1.1 mM, 0.7-1.2 mM, 0.8-1.3 mM, 0.9-1.4 mM, 1-1.5 mM, 1.1-1.6 mM, 1.2-1.7 mM, 1.3-1.8 mM, 1.4-1.9 mM, 1.5-2 mM, 1.6-2.1 mM, 1.7-2.2 mM, 1.8-2.3 mM, 1.9-2.4 mM, 2-2.5 mM, 2.1-2.6 mM, 2.2-2.7 mM, 2.3-2.8 mM, 2.4-2.9 mM, 2.5-3 mM, 2.6-3.1 mM, 2.7-3.2 mM, 2.8-3.3 mM, 2.9-3.4 mM, 3-3.5 mM, 3.1-3.6 mM, 3.2-3.7 mM, 3.3-3.8 mM, 3.4-3.9 mM, 3.5-4 mM, 3.6-4.1 mM, 3.7-4.2 mM, 3.8-4.3 mM, 3.9-4.4 mM, 4-4.5 mM, 4.1-4.6 mM, 4.2-4.7 mM, 4.3-4.8 mM, 4.4-4.9 mM, 4.5-5 mM, 4.6-5.1 mM, 4.7-5.2 mM, 4.8-5.3 mM, 4.9-5.4 mM, 5-5.5 mM, 5.1-5.6 mM, 5.2-5.7 mM, 5.3-5.8 mM, 5.4-5.9 mM, 5.5-6 mM, 5.6-6.1 mM, 5.7-6.2 mM, 5.8-6.3 mM, 5.9-6.4 mM, 6-6.5 mM, 6.1-6.6 mM, 6.2-6.7 mM, 6.3-6.8 mM, 6.4-6.9 mM, 6.5-7 mM, 6.6-7.1 mM, 6.7-7.2 mM, 6.8-7.3 mM, 6.9-7.4 mM, 7-7.5 mM, 7.1-7.6 mM, 7.2-7.7 mM, 7.3-7.8 mM, 7.4-7.9 mM, 7.5-8 mM, 7.6-8.1 mM, 7.7-8.2 mM, 7.8-8.3 mM, 7.9-8.4 mM, 8-8.5 mM, 8.1-8.6 mM, 8.2-8.7 mM, 8.3-8.8 mM, 8.4-8.9 mM, 8.5-9 mM, 8.6-9.1 mM, 8.7-9.2 mM, 8.8-9.3 mM, 8.9-9.4 mM, 9-9.5 mM, 9.1-9.6 mM, 9.2-9.7 mM, 9.3-9.8 mM, 9.4-9.9 mM, 9.5-10 mM, 9.6-10.1 mM, 9.7-10.2 mM, 9.8-10.3 mM, 9.9-10.4 mM, 10-10.5 mM, 10.1-10.6 mM, 10.2-10.7 mM, 10.3-10.8 mM, 10.4-10.9 mM, 10.5-11 mM, 10.6-11.1 mM, 10.7-11.2 mM, 10.8-11.3 mM, 10.9-11.4 mM, 11-11.5 mM, 11.1-11.6 mM, 11.2-11.7 mM, 11.3-11.8 mM, 11.4-11.9 mM, 11.5-12 mM, 11.6-12.1 mM, 11.7-12.2 mM, 11.8-12.3 mM, 11.9-12.4 mM, 12-12.5 mM, 12.1-12.6 mM, 12.2-12.7 mM, 12.3-12.8 mM, 12.4-12.9 mM, 12.5-13 mM, 12.6-13.1 mM, 12.7-13.2 mM, 12.8-13.3 mM, 12.9-13.4 mM, 13-13.5 mM, 13.1-13.6 mM, 13.2-13.7 mM, 13.3-13.8 mM, 13.4-13.9 mM, 13.5-14 mM, 13.6-14.1 mM, 13.7-14.2 mM, 13.8-14.3 mM, 13.9-14.4 mM, 14-14.5 mM, 14.1-14.6 mM, 14.2-14.7 mM, 14.3-14.8 mM, 14.4-14.9 mM, 14.5-15 mM, 0-1 mM, 1-2 mM, 2-3 mM, 3-4 mM, 4-5 mM, 5-6 mM, 6-7 mM, 7-8 mM, 8-9 mM, 9-10 mM, 10-11 mM, 11-12 mM, 12-13 mM, 13-14 mM, 14-15 mM, 15-16 mM, 0-2 mM, 1-3 mM, 2-4 mM, 3-5 mM, 4-6 mM, 5-7 mM, 6-8 mM, 7-9 mM, 8-10 mM, 9-11 mM, 10-12 mM, 11-13 mM, 12-14 mM, 13-15 mM, 0-3 mM, 1-4 mM, 2-5 mM, 3-6 mM, 4-7 mM, 5-8 mM, 6-9 mM, 7-10 mM, 8-11 mM, 9-12 mM, 10-13 mM, 11-14 mM, 12-15 mM, 0-4 mM, 1-5 mM, 2-6 mM, 3-7 mM, 4-8 mM, 5-9 mM, 6-10 mM, 7-11 mM, 8-12 mM, 9-13 mM, 10-14 mM, 11-15 mM, 0-5 mM, 1-6 mM, 2-7 mM, 3-8 mM, 4-9 mM, 5-10 mM, 6-11 mM, 7-12 mM, 8-13 mM, 9-14 mM, 10-15 mM, 0-6 mM, 1-7 mM, 2-8 mM, 3-9 mM, 4-10 mM, 5-11 mM, 6-12 mM, 7-13 mM, 8-14 mM, 9-15 mM, 0-7 mM, 1-8 mM, 2-9 mM, 3-10 mM, 4-11 mM, 5-12 mM, 6-13 mM, 7-14 mM, 8-15 mM, 0-8 mM, 1-9 mM, 2-10 mM, 3-11 mM, 4-12 mM, 5-13 mM, 6-14 mM, 7-15 mM, 0-9 mM, 1-10 mM, 2-11 mM, 3-12 mM, 4-13 mM, 5-14 mM, 6-15 mM, 0-10 mM, 1-11 mM, 2-12 mM, 3-13 mM, 4-14 mM, 5-15 mM, 0-11 mM, 1-12 mM, 2-13 mM, 3-14 mM, 4-15 mM, 0-12 mM, 1-13 mM, 2-14 mM, 3-15 mM, 0-13 mM, 1-14 mM, 2-15 mM, 0-14 mM, 1-15 mM, or 0-15 mM.

In certain embodiments, the formulation may include 0-10 mM of hydrochloric acid.

In certain embodiments, the formulation may include 6.2-6.3 mM of hydrochloric acid.

In certain embodiments, the formulation may include 8.9-9 mM of hydrochloric acid.

In certain embodiments, the formulation may include 6.2 mM of hydrochloric acid.

In certain embodiments, the formulation may include 6.3 mM of hydrochloric acid.

In certain embodiments, the formulation may include 8.9 mM of hydrochloric acid.

In certain embodiments, the formulation may include 9 mM of hydrochloric acid.

### Sugar

In certain embodiments, the formulation may include at least one sugar and/or sugar substitute.

In certain embodiments, the formulation may include at least one sugar and/or sugar substitute to increase the stability of the formulation. This increase in stability may provide longer hold times for in-process pools, provide a longer “shelf-life”, increase the concentration of AAV particles in solution (e.g., the formulation is able to have higher concentrations of AAV particles without rAAV dropping out of the solution) and/or reduce the generation or formation of aggregation in the formulations. In certain embodiments, the inclusion of at least one sugar and/or sugar substitute in the formulation may increase the stability of the formulation by 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or more than 95%, 1-5%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%. 10-50%, 10-55%, 10-60%, 10-65%, 10-70%, 10-75%, 10-80%, 10-85%, 10-90%, 10-95%, 15-25%, 15-30%, 15-35%, 1540%, 1545%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95% as compared to the same formulation without the sugar and/or sugar substitute.

In certain embodiments, the sugar and/or sugar substitute is used in combination with a phosphate buffer for increased stability. The combination of the sugar and/or sugar substitute with the phosphate butter may increase stability by 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or more than 95%, 1-5%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95% as compared to the same formulation without the sugar and/or sugar substitute. As a non-limiting example, the sugar is sucrose. As another non-limiting example, the sugar is trehalose. As another non-limiting example, the sugar substitute is sorbitol.

In certain embodiments, the hold time of the formulation may be increased by 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or more than 95%, 1-5%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95% as compared to the same formulation without the sugar and/or sugar substitute.

In certain embodiments, the shelf-life of the formulation may be increased by 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or more than 95%, 1-5%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95% as compared to the same formulation without the sugar and/or sugar substitute. The shelf-life may be 1, 2, 3, 4, 5, 6, 7, 8, 9, 10, 11, 12, 13, 14, 15, 16, 17, 18, 19, 20, 21, 22, 23, 24 hours, or 1, 2, 3, 4 weeks, or 1, 2, 3, 4, 5, 6, 7, 8, 9, 10, 11, 12, 13, 14, 15, 16, 17, 18, 19, 20, 21, 22, 23, 24 months, or 1, 2, 3, 4, 5, 6, 7 or more than 7 years.

In certain embodiments, the concentration of the AAV particles in the formulation may be increased by 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or more than 95%, 1-5%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 1540%, 1545%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35% 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95% as compared to the same formulation without the sugar and/or sugar substitute.

In certain embodiments, as a result of the addition of a sugar and/or sugar substitute, the formulation or generation of aggregates in the formulation may be reduced by 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or more than 95%, 1-5%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%/a, 10-55%/a, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95% as compared to the same formulation without the sugar and/or sugar substitute.

In certain embodiments, as a result of the addition of a sugar and/or sugar substitute, the formulation or generation of aggregates may be 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or more than 95%, 1-5%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95% as determined by a method known in the art (e.g., by DLS measurement) and as compared to the same formulation without the sugar and/or sugar substitute. As a non-limiting example, the aggregation of a formulation can be less than 2% by the addition of at least one sugar and/or sugar substitute to the formulation. Additional aggregates can be removed by methods known in the art.

In certain embodiments, the formulation may include a sugar and/or sugar substitute at 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.70%, 3.8%, 3.9%, 40%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, or 10% w/v.

In certain embodiments, the formulation may include a sugar and/or sugar substitute in a range of 0-1%, 0.1-1%, 0.2-1%, 0.3-1%, 0.4-1%, 0.5-1%, 0.6-1%, 0.7-1%, 0.8-1%, 0.9-1%, 0-1.5%, 0.1-1.5%, 0.2-1.5%, 0.3-1.5%, 0.4-1.5%, 0.5-1.5%, 0.6-1.5%, 0.7-1.5%, 0.8-1.5%, 0.9-1.5%, 1-1.5%, 1.1-1.5%, 1.2-1.5%, 1.3-1.5%, 1.4-1.5%, 0-2%, 0.1-2%, 0.2-2%, 0.3-2%, 0.4-2%, 0.5-2%, 0.6-2%, 0.7-2%, 0.8-2%, 0.9-2%, 1-2%, 1.1-2%, 1.2-2%, 1.3-2%, 1.4-2%, 1.5-2%, 1.6-2%, 1.7-2%, 1.8-2%, 1.9-2%, 0-2.5%, 0.1-2.5%, 0.2-2.5%, 0.3-2.5%, 0.4-2.5%, 0.5-2.5%, 0.6-2.5%, 0.7-2.5%, 0.8-2.5%, 0.9-2.5%, 1-2.5%, 1.1-2.5%, 1.2-2.5%, 1.3-2.5%, 1.4-2.5%, 1.5-2.5%, 1.6-2.5%, 1.7-2.5%, 1.8-2.5%, 1.9-2.5%, 2-2.5%, 2.1-2.5%, 2.2-2.5%, 2.3-2.5%, 2.4-2.5%, 0-3%, 0.1-3%, 0.2-3%, 0.3-3%, 0.4-3%, 0.5-3%, 0.6-3%, 0.7-3%, 0.8-3%, 0.9-3%, 1-3%, 1.1-3%, 1.2-3%, 1.3-3%, 1.4-3%, 1.5-3%, 1.6-3%, 1.7-3%, 1.8-3%, 1.9-3%, 2-3%, 2.1-3%, 2.2-3%, 2.3-3%, 2.4-3%, 2.5-3%, 2.6-3%, 2.7-3%, 2.8-3%, 2.9-3%, 0-3.5%, 0.1-3.5%, 0.2-3.5%, 0.3-3.5%, 0.4-3.5%, 0.5-3.5%, 0.6-3.5%, 0.7-3.5%, 0.8-3.5%, 0.9-3.5%, 1-3.5%, 1.1-3.5%, 1.2-3.5%, 1.3-3.5%, 1.4-3.5%, 1.5-3.5%, 1.6-3.5/0, 1.7-3.5%, 1.8-3.5%, 1.9-3.5%, 2-3.5%, 2.1-3.5%, 2.2-3.5%, 2.3-3.5%, 2.4-3.5%, 2.5-3.5%, 2.6-3.5%, 2.7-3.5%, 2.8-3.5%, 2.9-3.5%, 3-3.5%, 3.1-3.5%, 3.2-3.5%, 3.3-3.5%, 3.4-3.5%, 0-4%, 0.1-4%, 0.2-4%, 0.34%, 0.4-4%, 0.54%, 0.6-4%, 0.7-4%, 0.8-4%, 0.9-4%, 1-4%, 1.1-4%, 1.2-4%, 1.3-4%, 1.4-4%, 1.5-4%, 1.64%, 1.7-4%, 1.84%, 1.9-4%, 24%, 2.1-4%, 2.2-4%, 2.3-4%, 2.4-4%, 2.5-4%, 2.6-4%, 2.74%, 2.8-4%, 2.94%, 34%, 3.1-4%, 3.2-4%, 3.3-4%, 3.4-4%, 3.54%, 3.6-4%, 3.74%, 3.8-4%, 3.9-4%, 0-4.5%, 0.1-4.5%, 0.2-4.5%, 0.3-4.5%, 0.4-4.5%, 0.5-4.5%, 0.6-4.5%, 0.7-4.5%, 0.8-4.5%, 0.9-4.5%, 1-4.5%, 1.1-4.5%, 1.2-4.5%, 1.3-4.5%, 1.4-4.5%, 1.5-4.5%, 1.6-4.5%, 1.7-4.5%, 1.8-4.5%, 1.9-4.5%, 2-4.5%, 2.1-4.5%, 2.2-4.5%, 2.3-4.5%, 2.4-4.5%, 2.5-4.5%, 2.6-4.5%, 2.7-4.5%, 2.8-4.5%, 2.9-4.5%, 3-4.5%, 3.1-4.5%, 3.2-4.5%, 3.3-4.5%, 3.4-4.5%, 3.5-4.5%, 3.6-4.5%, 3.7-4.5%, 3.8-4.5%, 3.9-4.5%, 4-4.5%, 4.1-4.5%, 4.2-4.5%, 4.3-4.5%, 4.4-4.5%, 0-5%, 0.1-5%, 0.2-5%, 0.3-5%, 0.4-5%, 0.5-5%, 0.6-5%, 0.7-5%, 0.8-5%, 0.9-5%, 1-5%, 1.1-5%, 1.2-5%, 1.3-5%, 1.4-5%, 1.5-5%, 1.6-5%, 1.7-5%, 1.8-5%, 1.9-5%, 2-5%, 2.1-5%, 2.2-5%, 2.3-5%, 2.4-5%, 2.5-5%, 2.6-5%, 2.7-5%, 2.8-5%, 2.9-5%, 3-5%, 3.1-5%, 3.2-5%, 3.3-5%, 3.4-5%, 3.5-5%, 3.6-5%, 3.7-5%, 3.8-5%, 3.9-5%, 4-5%, 4.1-5%, 4.2-5%, 4.3-5%, 4.4-5%, 4.5-5%, 4.6-5%, 4.7-5%, 4.8-5%, 4.9-5%, 0-5.5%, 0.1-5.5%, 0.2-5.5%, 0.3-5.5%, 0.4-5.5%, 0.5-5.5%, 0.6-5.5%, 0.7-5.5%. 0.8-5.5%, 0.9-5.5%, 1-5.5%, 1.1-5.5%, 1.2-5.5%, 1.3-5.5%, 1.4-5.5%, 1.5-5.5%, 1.6-5.5%, 1.7-5.5%, 1.8-5.5%, 1.9-5.5%, 2-5.5%, 2.1-5.5%, 2.2-5.5%, 2.3-5.5%, 2.4-5.5%, 2.5-5.5%, 2.6-5.5%, 2.7-5.5%, 2.8-5.5%, 2.9-5.5%, 3-5.5%, 3.1-5.5%, 3.2-5.5%, 3.3-5.5%, 3.4-5.5%, 3.5-5.5%, 3.6-5.5%, 3.7-5.5%, 3.8-5.5%, 3.9-5.5%, 4-5.5%, 4.1-5.5%, 4.2-5.5%, 4.3-5.5%, 4.4-5.5%, 4.5-5.5%, 4.6-5.5%, 4.7-5.5%, 4.8-5.5%, 4.9-5.5%, 5-5.5%, 5.1-5.5%, 5.2-5.5%, 5.3-5.5%, 5.4-5.5%, 0-6%, 0.1-6%, 0.2-6%, 0.3-6%, 0.4-6%, 0.5-6%, 0.6-6%, 0.7-6%, 0.8-6%, 0.9-6%, 1-6%, 1.1-6%, 1.2-6%, 1.3-6%, 1.4-6%, 1.5-6%, 1.6-6%, 1.7-6%, 1.8-6%, 1.9-6%, 2-6%, 2.1-6%, 2.2-6%, 2.3-6%, 2.4-6%, 2.5-6%, 2.6-6%, 2.7-6%, 2.8-6%, 2.9-6%, 3-6%, 3.1-6%, 3.2-6%, 3.3-6%, 3.4-6%, 3.5-6%, 3.6-6%, 3.7-6%, 3.8-6%, 3.9-6%, 4-6%, 4.1-6%, 4.2-6%, 4.3-6%, 4.4-6%, 4.5-6%, 4.6-6%, 4.7-6%, 4.8-6%, 4.9-6%, 5-6%, 5.1-6%, 5.2-6%, 5.3-6%, 5.4-6%, 5.5-6%, 5.6-6%, 5.7-6%, 5.8-6%, 5.9-6%, 0-6.5%, 0.1-6.5%, 0.2-6.5%, 0.3-6.5%, 0.4-6.5%, 0.5-6.5%, 0.6-6.5%, 0.7-6.5%, 0.8-6.5%, 0.9-6.5%, 1-6.5%, 1.1-6.5%, 1.2-6.5%, 1.3-6.5%, 1.4-6.5%, 1.5-6.5%, 1.6-6.5%, 1.7-6.5%, 1.8-6.5%, 1.9-6.5%, 2-6.5%, 2.1-6.5%, 2.2-6.5%, 2.3-6.5%, 2.4-6.5%, 2.5-6.5%, 2.6-6.5%, 2.7-6.5%, 2.8-6.5%, 2.9-6.5%, 3-6.5%, 3.1-6.5%, 3.2-6.5%, 3.3-6.5%, 3.4-6.5%, 3.5-6.5%, 3.6-6.5%, 3.7-6.5%, 3.8-6.5%, 3.9-6.5%, 4-6.5%, 4.1-6.5%, 4.2-6.5%, 4.3-6.5%, 4.4-6.5%, 4.5-6.5%, 4.6-6.5%, 4.7-6.5%, 4.8-6.5%, 4.9-6.5%, 5-6.5%, 5.1-6.5%, 5.2-6.5%, 5.3-6.5%, 5.4-6.5%, 5.5-6.5%, 5.6-6.5%, 5.7-6.5%, 5.8-6.5%, 5.9-6.5%, 6-6.5%, 6.1-6.5%, 6.2-6.5%, 6.3-6.5%, 6.4-6.5%, 0-7%, 0.1-7%, 0.2-7%, 0.3-7%, 0.4-7%, 0.5-7%, 0.6-7%, 0.7-7%, 0.8-7%, 0.9-7%, 1-7%, 1.1-7%, 1.2-7%, 1.3-7%, 1.4-7%, 1.5-7%, 1.6-7%, 1.7-7%, 1.8-7%, 1.9-7%, 2-7%, 2.1-7%, 2.2-7%, 2.3-7%, 2.4-7%, 2.5-7%, 2.6-7%, 2.7-7%, 2.8-7%, 2.9-7%, 3-7%, 3.1-7%, 3.2-7%, 3.3-7%, 3.4-7%, 3.5-7%, 3.6-7%, 3.7-7%, 3.8-7%, 3.9-7%, 4-7%, 4.1-7%, 4.2-7%, 4.3-7%, 4.4-7%, 4.5-7%, 4.6-7%, 4.7-7%, 4.8-7%, 4.9-7%, 5-7%, 5.1-7%, 5.2-7%, 5.3-7%, 5.4-7%, 5.5-7%, 5.6-7%, 5.7-7%, 5.8-7%, 5.9-7%, 6-7%, 6.1-7%, 6.2-7%, 6.3-7%, 6.4-7%, 6.5-7%, 6.6-7%, 6.7-7%, 6.8-7%, 6.9-7%, 0-7.5%, 0.1-7.5%, 0.2-7.5%, 0.3-7.5%, 0.4-7.5%, 0.5-7.5%, 0.6-7.5%, 0.7-7.5%, 0.8-7.5%, 0.9-7.5%, 1-7.5%, 1.1-7.5%, 1.2-7.5%, 1.3-7.5%, 1.4-7.5%, 1.5-7.5%, 1.6-7.5%, 1.7-7.5%, 1.8-7.5%, 1.9-7.5%, 2-7.5%, 2.1-7.5%, 2.2-7.5%, 2.3-7.5%, 2.4-7.5%, 2.5-7.5%, 2.6-7.5%, 2.7-7.5%, 2.8-7.5%, 2.9-7.5%, 3-7.5%, 3.1-7.5%, 3.2-7.5%, 3.3-7.5%, 3.4-7.5%, 3.5-7.5%, 3.6-7.5%, 3.7-7.5%, 3.8-7.5%, 3.9-7.5%, 4-7.5%, 4.1-7.5%, 4.2-7.5%, 4.3-7.5%, 4.4-7.5%, 4.5-7.5%, 4.6-7.5%, 4.7-7.5%, 4.8-7.5%, 4.9-7.5%, 5-7.5%, 5.1-7.5%, 5.2-7.5%, 5.3-7.5%, 5.4-7.5%, 5.5-7.5%, 5.6-7.5%, 5.7-7.5%, 5.8-7.5%, 5.9-7.5%, 6-7.5%, 6.1-7.5%, 6.2-7.5%, 6.3-7.5%, 6.4-7.5%, 6.5-7.5%, 6.6-7.5%, 6.7-7.5%, 6.8-7.5%, 6.9-7.5%, 7-7.5%, 7.1-7.5%, 7.2-7.5%, 7.3-7.5%, 7.4-7.5%, 0-8%, 0.1-8%, 0.2-8%, 0.3-8%, 0.4-8%, 0.5-8%, 0.6-8%, 0.7-8%, 0.8-8%, 0.9-8%, 1-8%, 1.1-8%, 1.2-8%, 1.3-8%, 1.4-8%, 1.5-8%, 1.6-8%, 1.7-8%, 1.8-8%, 1.9-8%, 2-8%, 2.1-8%, 2.2-8%, 2.3-8%, 2.4-8%, 2.5-8%, 2.6-8%, 2.7-8%, 2.8-8%, 2.9-8%, 3-8%, 3.1-8%, 3.2-8%, 3.3-8%, 3.4-8%, 3.5-8%, 3.6-8%, 3.7-8%, 3.8-8%, 3.9-8%, 4-8%, 4.1-8%, 4.2-8%, 4.3-8%, 4.4-8%, 4.5-8%, 4.6-8%, 4.7-8%, 4.8-8%, 4.9-8%, 5-8%, 5.1-8%, 5.2-8%, 5.3-8%, 5.4-8%, 5.5-8%, 5.6-8%, 5.7-8%, 5.8-8%, 5.9-8%, 6-8%, 6.1-8%, 6.2-8%, 6.3-8%, 6.4-8%, 6.5-8%, 6.6-8%, 6.7-8%, 6.8-8%, 6.9-8%, 7-8%, 7.1-8%, 7.2-8%, 7.3-8%, 7.4-8%, 7.5-8%, 7.6-8%, 7.7-8%, 7.8-8%, 7.9-8%, 0-8.5%, 0.1-8.5%, 0.2-8.5%, 0.3-8.5%, 0.4-8.5%, 0.5-8.5%, 0.6-8.5%, 0.7-8.5%, 0.8-8.5%, 0.9-8.5%, 1-8.5%, 1.1-8.5%, 1.2-8.5%, 1.3-8.5%, 1.4-8.5%, 1.5-8.5%, 1.6-8.5%, 1.7-8.5%, 1.8-8.5%, 1.9-8.5%, 2-8.5%, 2.1-8.5%, 2.2-8.5%, 2.3-8.5%, 2.4-8.5/0, 2.5-8.5%, 2.6-8.5%, 2.7-8.5%, 2.8-8.5%, 2.9-8.5%, 3-8.5%, 3.1-8.5%, 3.2-8.5%, 3.3-8.5%, 3.4-8.5%, 3.5-8.5%, 3.6-8.5%, 3.7-8.5%, 3.8-8.5%, 3.9-8.5%, 4-8.5%, 4.1-8.5%, 4.2-8.5%, 4.3-8.5%, 4.4-8.5%, 4.5-8.5%, 4.6-8.5%, 4.7-8.5%, 4.8-8.5%, 4.9-8.5%, 5-8.5%, 5.1-8.5%, 5.2-8.5%, 5.3-8.5%, 5.4-8.5%, 5.5-8.5%, 5.6-8.5%, 5.7-8.5%, 5.8-8.5%, 5.9-8.5%, 6-8.5%, 6.1-8.5%, 6.2-8.5%, 6.3-8.5%, 6.4-8.5%, 6.5-8.5%, 6.6-8.5%, 6.7-8.5%, 6.8-8.5%, 6.9-8.5%, 7-8.5%, 7.1-8.5%, 7.2-8.5%, 7.3-8.5%, 7.4-8.5%, 7.5-8.5%, 7.6-8.5%, 7.7-8.5%, 7.8-8.5%, 7.9-8.5%, 8-8.5%, 8.1-8.5%, 8.2-8.5%, 8.3-8.5%, 8.4-8.5%, 0-9%, 0.1-9%, 0.2-9%, 0.3-9%, 0.4-9%, 0.5-9%, 0.6-9%, 0.7-9%, 0.8-9%, 0.9-9%, 1-9%, 1.1-9%, 1.2-9%, 1.3-9%, 1.4-9%, 1.5-9%, 1.6-9%, 1.7-9%, 1.8-9%, 1.9-9%, 2-9%, 2.1-9%, 2.2-9%, 2.3-9%, 2.4-9%, 2.5-9%, 2.6-9%, 2.7-9%, 2.8-9%, 2.9-9%, 3-9%, 3.1-9%, 3.2-9%, 3.3-9%, 3.4-9%, 3.5-9%, 3.6-9%, 3.7-9%, 3.8-9%, 3.9-9%, 4-9%, 4.1-9%, 4.2-9%, 4.3-9%, 4.4-9%, 4.5-9%, 4.6-9%, 4.7-9%, 4.8-9%, 4.9-9%, 5-9%, 5.1-9%, 5.2-9%, 5.3-9%, 5.4-9%, 5.5-9%, 5.6-9%, 5.7-9%, 5.8-9%, 5.9-9%, 6-9%, 6.1-9%, 6.2-9%, 6.3-9%, 6.4-9%, 6.5-9%, 6.6-9%, 6.7-9%, 6.8-9%, 6.9-9%, 7-9%, 7.1-9%, 7.2-9%, 7.3-9%, 7.4-9%, 7.5-9%, 7.6-9%, 7.7-9%, 7.8-9%, 7.9-9%, 8-9%, 8.1-9%, 8.2-9%, 8.3-9%, 8.4-9%, 8.5-9%, 8.6-9%, 8.7-9%, 8.8-9%, 8.9-9%, 0-9.5%, 0.1-9.5%, 0.2-9.5%, 0.3-9.5%, 0.4-9.5%, 0.5-9.5%, 0.6-9.5%, 0.7-9.5%, 0.8-9.5%, 0.9-9.5%, 1-9.5%, 1.1-9.5%, 1.2-9.5%, 1.3-9.5%, 1.4-9.5%, 1.5-9.5%, 1.6-9.5%, 1.7-9.5%, 1.8-9.5%, 1.9-9.5%, 2-9.5%, 2.1-9.5%, 2.2-9.5%, 2.3-9.5%, 2.4-9.5%, 2.5-9.5%, 2.6-9.5%, 2.7-9.5%, 2.8-9.5%, 2.9-9.5%, 3-9.5%, 3.1-9.5%, 3.2-9.5%, 3.3-9.5%, 3.4-9.5%, 3.5-9.5%, 3.6-9.5%, 3.7-9.5%, 3.8-9.5%, 3.9-9.5%, 4-9.5%, 4.1-9.5%, 4.2-9.5%, 4.3-9.5%, 4.4-9.5%, 4.5-9.5%, 4.6-9.5%, 4.7-9.5%, 4.8-9.5%, 4.9-9.5%, 5-9.5%, 5.1-9.5%, 5.2-9.5%, 5.3-9.5%, 5.4-9.5%, 5.5-9.5%, 5.6-9.5%, 5.7-9.5%, 5.8-9.5%, 5.9-9.5%, 6-9.5%, 6.1-9.5%, 6.2-9.5%, 6.3-9.5%, 6.4-9.5%, 6.5-9.5%, 6.6-9.5%, 6.7-9.5%, 6.8-9.5%, 6.9-9.5%, 7-9.5%, 7.1-9.5%, 7.2-9.5%, 7.3-9.5%, 7.4-9.5%, 7.5-9.5%, 7.6-9.5%, 7.7-9.5%, 7.8-9.5%, 7.9-9.5%, 8-9.5%, 8.1-9.5%, 8.2-9.5%, 8.3-9.5%, 8.4-9.5%, 8.5-9.5%, 8.6-9.5%, 8.7-9.5%, 8.8-9.5%, 8.9-9.5%, 9-9.5%, 9.1-9.5%, 9.2-9.5%, 9.3-9.5%, 9.4-9.5%, 0-10%, 0.1-10%, 0.2-10%, 0.3-10%, 0.4-10%, 0.5-10%, 0.6-10%, 0.7-10%, 0.8-10%, 0.9-10%, 1-10%, 1.1-10%, 1.2-10%, 1.3-10%, 1.4-10%, 1.5-10%, 1.6-10%, 1.7-10%, 1.8-10%, 1.9-10%, 2-10%, 2.1-10%, 2.2-10%, 2.3-10%, 2.4-10%, 2.5-10%, 2.6-10%, 2.7-10%, 2.8-10%, 2.9-10%, 3-10%, 3.1-10%, 3.2-10%, 3.3-10%, 3.4-10%, 3.5-10%, 3.6-10%, 3.7-10%, 3.8-10%, 3.9-10%, 4-10%, 4.1-10%, 4.2-10%, 4.3-10%, 4.4-10%, 4.5-10%, 4.6-10%, 4.7-10%, 4.8-10%, 4.9-10%, 5-10%, 5.1-10%, 5.2-10%, 5.3-10%, 5.4-10%, 5.5-10%, 5.6-10%, 5.7-10%, 5.8-10%, 5.9-10%, 6-10%, 6.1-10%, 6.2-10%, 6.3-10%, 6.4-10%, 6.5-10%, 6.6-10%, 6.7-10%, 6.8-10%, 6.9-10%, 7-10%, 7.1-10%, 7.2-10%, 7.3-10%, 7.4-10%, 7.5-10%, 7.6-10%, 7.7-10%, 7.8-10%, 7.9-10%, 8-10%, 8.1-10%, 8.2-10%, 8.3-10%, 8.4-10%, 8.5-10%, 8.6-10%, 8.7-10%, 8.8-10%, 8.9-10%, 9-10%, 9.1-10%, 9.2-10%, 9.3-10%, 9.4-10%, 9.5-10%, 9.6-10%, 9.7-10%, 9.8-10%, or 9.9-10% w/v.

In certain embodiments, the formulation may include 0-10% w/v of a sugar and/or sugar substitute.

In certain embodiments, the formulation may include 0-9% w/v of a sugar and/or sugar substitute.

In certain embodiments, the formulation may include 1% w/v of a sugar and/or sugar substitute.

In certain embodiments, the formulation may include 2% w/v of a sugar and/or sugar substitute.

In certain embodiments, the formulation may include 3% w/v of a sugar and/or sugar substitute.

In certain embodiments, the formulation may include 4% w/v of a sugar and/or sugar substitute.

In certain embodiments, the formulation may include 5% w/v of a sugar and/or sugar substitute.

In certain embodiments, the formulation may include 6% w/v of a sugar and/or sugar substitute.

In certain embodiments, the formulation may include 7% w/v of a sugar and/or sugar substitute.

In certain embodiments, the formulation may include 8% w/v of a sugar and/or sugar substitute.

In certain embodiments, the formulation may include 9% w/v of a sugar and/or sugar substitute.

In certain embodiments, the formulation may include 10% w/v of a sugar and/or sugar substitute.

In some embodiments, formulations of pharmaceutical compositions described herein may comprise a disaccharide. Suitable disaccharides that may be used in the formulation described herein may include sucrose, lactulose, lactose, maltose, trehalose, cellobiose, chitobiose, kojibiose, nigerose, isomaltose, β,β-trehalose, α,β-trehalose, sophorose, laminaribiose, gentiobiose, turanose, maltulose, palatinose, gentiobiulose, mannobiose, melibiose, melibiulose, rutinose, rutinulose, and xylobiose. The concentration of disaccharide (w/v) used in the formulation may be between 1%-15%, for example, between 1%-5%, between 3%-6%, between 5%-8%, between 7%-10%, or between 10%-15%.

In some embodiments, formulations of pharmaceutical compositions described herein may comprise a sugar alcohol. As a non-limiting example, the sugar alcohol that may be used in the formulation described herein may include sorbitol. The concentration of sugar alcohol (w/v) used in the formulation may be between 1%-15%, for example, between 1%-5%, between 3%-6%, between 5%-8%, between 7%-10%, or between 10%-15%.

### Sucrose

In certain embodiments, the formulation may include at least one sugar which is disaccharide such as, but not limited to, sucrose.

In certain embodiments, the formulation may include sucrose at 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%. 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, or 10% w/v.

In certain embodiments, the formulation may include sucrose in a range of 0-1%, 0.1-1%, 0.2-1%, 0.3-1%, 0.4-1%, 0.5-1%, 0.6-1%, 0.7-1%, 0.8-1%, 0.9-1%, 0-1.5%, 0.1-1.5%, 0.2-1.5%, 0.3-1.5%, 0.4-1.5%, 0.5-1.5%, 0.6-1.5%, 0.7-1.5%, 0.8-1.5%, 0.9-1.5%, 1-1.5%, 1.1-1.5%, 1.2-1.5%, 1.3-1.5%, 1.4-1.5%, 0-2%, 0.1-2%, 0.2-2%, 0.3-2%, 0.4-2%, 0.5-2%, 0.6-2%, 0.7-2%, 0.8-2%, 0.9-2%, 1-2%, 1.1-2%, 1.2-2%, 1.3-2%, 1.4-2%, 1.5-2%, 1.6-2%, 1.7-2%, 1.8-2%, 1.9-2%, 0-2.5%, 0.1-2.5%, 0.2-2.5%, 0.3-2.5%, 0.4-2.5%, 0.5-2.5%, 0.6-2.5%, 0.7-2.5%, 0.8-2.5%, 0.9-2.5%, 1-2.5%, 1.1-2.5%, 1.2-2.5%, 1.3-2.5%, 1.4-2.5%, 1.5-2.5%, 1.6-2.5%, 1.7-2.5%, 1.8-2.5%, 1.9-2.5%, 2-2.5%, 2.1-2.5%, 2.2-2.5%, 2.3-2.5%, 2.4-2.5%, 0-3%, 0.1-3%, 0.2-3%, 0.3-3%, 0.4-3%, 0.5-3%, 0.6-3%, 0.7-3%, 0.8-3%, 0.9-3%. 1-3%, 1.1-3%, 1.2-3%, 1.3-3%, 1.4-3%, 1.5-3%, 1.6-3%, 1.7-3%, 1.8-3%, 1.9-3%, 2-3%, 2.1-3%, 2.2-3%, 2.3-3%, 2.4-3%, 2.5-3%, 2.6-3%, 2.7-3%, 2.8-3%, 2.9-3%, 0-3.5%, 0.1-3.5%, 0.2-3.5%, 0.3-3.5%, 0.4-3.5%, 0.5-3.5%, 0.6-3.5%, 0.7-3.5%, 0.8-3.5%, 0.9-3.5%, 1-3.5%, 1.1-3.5%, 1.2-3.5%, 1.3-3.5%, 1.4-3.5%, 1.5-3.5%, 1.6-3.5%, 1.7-3.5%, 1.8-3.5%, 1.9-3.5%, 2-3.5%, 2.1-3.5%, 2.2-3.5%, 2.3-3.5%, 2.4-3.5%, 2.5-3.5%, 2.6-3.5%, 2.7-3.5%, 2.8-3.5%, 2.9-3.5%, 3-3.5%, 3.1-3.5%, 3.2-3.5%, 3.3-3.5%, 3.4-3.5%, 0-4%, 0.14%, 0.2-4%, 0.3-4%, 0.4-4%, 0.5-4%, 0.6-4%, 0.7-4%, 0.8-4%, 0.9-4%, 1-4%, 1.1-4%, 1.24%, 1.3-4%, 1.4-4%, 1.5-4%, 1.6-4%, 1.7-4%, 1.8-4%, 1.9-4%, 2-4%, 2.1-4%, 2.2-4%, 2.3%, 2.4-4%, 2.5-4%, 2.6-4%, 2.7-4%, 2.8-4%, 2.9-4%, 3-4%, 3.1-4%, 3.2-4%, 3.3-4%, 3.4-4%, 3.5-4%, 3.6-4%, 3.7-4%, 3.8-4%, 3.9-4%, 0-4.5%, 0.1-4.5%, 0.2-4.5%, 0.3-4.5%, 0.4-4.5%, 0.5-4.5%, 0.6-4.5%, 0.7-4.5%, 0.8-4.5%, 0.9-4.5%, 1-4.5%, 1.1-4.5%, 1.2-4.5%, 1.3-4.5%, 1.4-4.5%, 1.5-4.5%, 1.6-4.5%, 1.7-4.5%, 1.8-4.5%, 1.9-4.5%, 2-4.5%, 2.1-4.5%, 2.2-4.5%, 2.3-4.5%, 2.4-4.5%, 2.5-4.5%, 2.6-4.5%, 2.7-4.5%, 2.8-4.5%, 2.9-4.5%, 3-4.5%, 3.1-4.5%, 3.2-4.5%, 3.3-4.5%, 3.4-4.5%, 3.5-4.5%, 3.6-4.5%, 3.7-4.5%, 3.8-4.5%, 3.9-4.5%, 4-4.5%, 4.1-4.5%, 4.2-4.5%, 4.3-4.5%, 4.4-4.5%, 0-5%, 0.1-5%, 0.2-5%, 0.3-5%, 0.4-5%, 0.5-5%, 0.6-5%, 0.7-5%, 0.8-5%, 0.9-5%, 1-5,%, 1.1-5%, 1.2-5,%, 1.3-5%, 1.4-5%, 1.5-5%, 1.6-5%, 1.7-5%, 1.8-5%, 1.9-5%, 2-5%, 2.1-5%, 2.2-5%, 2.3-5%, 2.4-5%, 2.5-5%, 2.6-5%, 2.7-5%, 2.8-5%, 2.9-5%, 3-5%, 3.1-5%, 3.2-5%, 3.3-5%, 3.4-5%, 3.5-5%, 3.6-5%, 3.7-5%, 3.8-5%, 3.9-5%, 4-5%, 4.1-5%, 4.2-5%, 4.3-5%, 4.4-5%, 4.5-5%, 4.6-5%, 4.7-5%, 4.8-5%, 4.9-5%, 0-5.5%, 0.1-5.5%, 0.2-5.5%, 0.3-5.5%, 0.4-5.5%, 0.5-5.5%, 0.6-5.5%, 0.7-5.5%, 0.8-5.5%, 0.9-5.5%, 1-5.5%, 1.1-5.5%, 1.2-5.5%, 1.3-5.5%, 1.4-5.5%, 1.5-5.5%, 1.6-5.5%, 1.7-5.5%, 1.8-5.5%, 1.9-5.5%, 2-5.5%, 2.1-5.5%, 2.2-5.5%, 2.3-5.5%, 2.4-5.5%, 2.5-5.5%, 2.6-5.5%, 2.7-5.5%, 2.8-5.5%, 2.9-5.5%, 3-5.5%, 3.1-5.5%, 3.2-5.5%, 3.3-5.5%, 3.4-5.5%, 3.5-5.5%, 3.6-5.5%, 3.7-5.5%, 3.8-5.5%, 3.9-5.5%, 4-5.5%, 4.1-5.5%, 4.2-5.5%, 4.3-5.5%, 4.4-5.5%, 4.5-5.5%, 4.6-5.5%, 4.7-5.5%, 4.8-5.5%, 4.9-5.5%, 5-5.5%, 5.1-5.5%, 5.2-5.5%, 5.3-5.5%, 5.4-5.5%, 0-6%, 0.1-6%, 0.2-6%, 0.3-6%, 0.4-6%, 0.5-6%, 0.6-6%, 0.7-6%, 0.8-6%, 0.9-6%, 1-6%, 1.1-6%, 1.2-6%, 1.3-6%, 1.4-6%, 1.5-6%, 1.6-6%, 1.7-6%, 1.8-6%, 1.9-6%, 2-6%, 2.1-6%, 2.2-6%, 2.3-6%, 2.4-6%, 2.5-6%, 2.6-6%, 2.7-6%, 2.8-6%, 2.9-6%, 3-6%, 3.1-6%, 3.2-6%, 3.3-6%, 3.4-6%, 3.5-6%, 3.6-6%, 3.7-6%, 3.8-6%, 3.9-6%, 4-6%, 4.1-6%, 4.2-6%, 4.3-6%, 4.4-6%, 4.5-6%, 4.6-6%, 4.7-6%, 4.8-6%, 4.9-6%, 5-6%, 5.1-6%, 5.2-6%, 5.3-6%, 5.4-6%, 5.5-6%, 5.6-6%, 5.7-6%, 5.8-6%, 5.9-6%, 0-6.5%, 0.1-6.5%, 0.2-6.5%, 0.3-6.5%, 0.4-6.5%, 0.5-6.5%, 0.6-6.5%, 0.7-6.5%, 0.8-6.5%, 0.9-6.5%, 1-6.5%, 1.1-6.5%, 1.2-6.5%, 1.3-6.5%, 1.4-6.5%, 1.5-6.5%, 1.6-6.5%, 1.7-6.5%, 1.8-6.5%, 1.9-6.5%, 2-6.5%, 2.1-6.5%, 2.2-6.5%, 2.3-6.5%, 2.4-6.5%, 2.5-6.5%, 2.6-6.5%, 2.7-6.5%, 2.8-6.5%, 2.9-6.5%, 3-6.5%, 3.1-6.5%, 3.2-6.5%, 3.3-6.5%, 3.4-6.5%, 3.5-6.5%, 3.6-6.5%, 3.7-6.5%, 3.8-6.5%, 3.9-6.5%, 4-6.5%, 4.1-6.5%, 4.2-6.5%, 4.3-6.5%, 4.4-6.5%, 4.5-6.5%, 4.6-6.5%, 4.7-6.5%, 4.8-6.5%, 4.9-6.5%, 5-6.5%, 5.1-6.5%, 5.2-6.5%, 5.3-6.5%, 5.4-6.5%, 5.5-6.5%, 5.6-6.5%, 5.7-6.5%, 5.8-6.5%, 5.9-6.5%, 6-6.5%, 6.1-6.5%, 6.2-6.5%, 6.3-6.5%, 6.4-6.5%, 0-7%, 0.1-7%, 0.2-7%, 0.3-7%, 0.4-7%, 0.5-7%, 0.6-7%, 0.7-7%, 0.8-7%, 0.9-7%, 1-7%, 1.1-7%, 1.2-7%, 1.3-7%, 1.4-7%, 1.5-7%, 1.6-7%, 1.7-7%, 1.8-7%, 1.9-7%, 2-7%, 2.1-7%, 2.2-7%, 2.3-7%, 2.4-7%, 2.5-7%, 2.6-7%, 2.7-7%, 2.8-7%, 2.9-7%, 3-7%, 3.1-7%, 3.2-7%, 3.3-7%, 3.4-7%, 3.5-7%, 3.6-7%, 3.7-7%, 3.8-7%, 3.9-7%, 4-7%, 4.1-7%, 4.2-7%, 4.3-7%, 4.4-7%, 4.5-7%, 4.6-7%, 4.7-7%, 4.8-7%, 4.9-7%, 5-7%, 5.1-7%, 5.2-7%, 5.3-7%, 5.4-7%, 5.5-7%, 5.6-7%, 5.7-7%, 5.8-7%, 5.9-7%, 6-7%, 6.1-7%, 6.2-7%, 6.3-7%, 6.4-7%, 6.5-7%, 6.6-7%, 6.7-7%, 6.8-7%, 6.9-7%, 0-7.5%, 0.1-7.5%, 0.2-7.5%, 0.3-7.5%, 0.4-7.5%, 0.5-7.5%, 0.6-7.5%, 0.7-7.5%, 0.8-7.5%, 0.9-7.5%, 1-7.5%, 1.1-7.5%, 1.2-7.5%, 1.3-7.5%, 1.4-7.5%, 1.5-7.5%, 1.6-7.5%, 1.7-7.5%, 1.8-7.5%, 1.9-7.5%, 2-7.5%, 2.1-7.5%, 2.2-7.5%, 2.3-7.5%, 2.4-7.5%, 2.5-7.5/a, 2.6-7.5%, 2.7-7.5%, 2.8-7.5%, 2.9-7.5%, 3-7.5%, 3.1-7.5%, 3.2-7.5%, 3.3-7.5%, 3.4-7.5%, 3.5-7.5%, 3.6-7.5%, 3.7-7.5%, 3.8-7.5%, 3.9-7.5%, 4-7.5%, 4.1-7.5%, 4.2-7.5%, 4.3-7.5%, 4.4-7.5%, 4.5-7.5%, 4.6-7.5%, 4.7-7.5%, 4.8-7.5%, 4.9-7.5%, 5-7.5%, 5.1-7.5%, 5.2-7.5%, 5.3-7.5%, 5.4-7.5%, 5.5-7.5%, 5.6-7.5%, 5.7-7.5%, 5.8-7.5%, 5.9-7.5%, 6-7.5%, 6.1-7.5%, 6.2-7.5%, 6.3-7.5%, 6.4-7.5%, 6.5-7.5%, 6.6-7.5%, 6.7-7.5%, 6.8-7.5%, 6.9-7.5%, 7-7.5/0, 7.1-7.5%, 7.2-7.5%, 7.3-7.5%, 7.4-7.5%, 0-8%, 0.1-8%, 0.2-8%, 0.3-8%, 0.4-8%, 0.5-8%, 0.6-8%, 0.7-8%, 0.8-8%, 0.9-8%, 1-8%, 1.1-8%, 1.2-8%, 1.3-8%, 1.4-8%, 1.5-8%, 1.6-8%, 1.7-8%, 1.8-8%, 1.9-8%, 2-8%, 2.1-8%, 2.2-8%, 2.3-8%, 2.4-8%, 2.5-8%, 2.6-8%, 2.7-8%, 2.8-8%, 2.9-8%, 3-8%, 3.1-8%, 3.2-8%, 3.3-8%, 3.4-8%, 3.5-8%, 3.6-8%, 3.7-8%, 3.8-8%, 3.9-8%, 4-8%, 4.1-8%, 4.2-8%, 4.3-8%, 4.4-8%, 4.5-8%, 4.6-8%, 4.7-8%, 4.8-8%, 4.9-8%, 5-8%, 5.1-8%, 5.2-8%, 5.3-8%, 5.4-8%, 5.5-8%, 5.6-8%, 5.7-8%, 5.8-8%, 5.9-8%, 6-8%, 6.1-8%, 6.2-8%, 6.3-8%, 6.4-8%, 6.5-8%, 6.6-8%, 6.7-8%, 6.8-8%, 6.9-8%, 7-8%, 7.1-8%, 7.2-8%, 7.3-8%, 7.4-8%, 7.5-8%, 7.6-8%, 7.7-8%, 7.8-8%, 7.9-8%, 0-8.5%, 0.1-8.5%, 0.2-8.5%. 0.3-8.5%, 0.4-8.5%, 0.5-8.5%, 0.6-8.5%, 0.7-8.5%, 0.8-8.5%, 0.9-8.5%, 1-8.5%, 1.1-8.5%, 1.2-8.5%, 1.3-8.5%, 1.4-8.5%, 1.5-8.5%, 1.6-8.5%, 1.7-8.5%, 1.8-8.5%, 1.9-8.5%, 2-8.5%, 2.1-8.5%, 2.2-8.5%, 2.3-8.5%, 2.4-8.5%, 2.5-8.5%, 2.6-8.5%, 2.7-8.5%, 2.8-8.5/0, 2.9-8.5%, 3-8.5%, 3.1-8.5%, 3.2-8.5%, 3.3-8.5%, 3.4-8.5%, 3.5-8.5%, 3.6-8.5%, 3.7-8.5%, 3.8-8.5%, 3.9-8.5%, 4-8.5%, 4.1-8.5%, 4.2-8.5%, 4.3-8.5%, 4.4-8.5%, 4.5-8.5%, 4.6-8.5%, 4.7-8.5%, 4.8-8.5%, 4.9-8.5%, 5-8.5%, 5.1-8.5%, 5.2-8.5%, 5.3-8.5%, 5.4-8.5%, 5.5-8.5%, 5.6-8.5%, 5.7-8.5%, 5.8-8.5%, 5.9-8.5%, 6-8.5%, 6.1-8.5%, 6.2-8.5%, 6.3-8.5%, 6.4-8.5%, 6.5-8.5%, 6.6-8.5%, 6.7-8.5%, 6.8-8.5%, 6.9-8.5%, 7-8.5%, 7.1-8.5%, 7.2-8.5%, 7.3-8.5%, 7.4-8.5%, 7.5-8.5%, 7.6-8.5%, 7.7-8.5%, 7.8-8.5%, 7.9-8.5%, 8-8.5%, 8.1-8.5%, 8.2-8.5%, 8.3-8.5%, 8.4-8.5%, 0-9%, 0.1-9%, 0.2-9%, 0.3-9%, 0.4-9%, 0.5-9%, 0.6-9%, 0.7-9%, 0.8-9%, 0.9-9%. 1-9%, 1.1-9%, 1.2-9%, 1.3-9%, 1.4-9%, 1.5-9%, 1.6-9%, 1.7-9%, 1.8-9%, 1.9-9%, 2-9%, 2.1-9%, 2.2-9%, 2.3-9%, 2.4-9%, 2.5-9%, 2.6-9%, 2.7-9%, 2.8-9%, 2.9-9%, 3-9%, 3.1-9%, 3.2-9%, 3.3-9%, 3.4-9%, 3.5-9%, 3.6-9%, 3.7-9%, 3.8-9%, 3.9-9% 4-9%, 4.1-9%, 4.2-9%, 4.3-9%, 4.4-9%, 4.5-9%, 4.6-9%, 4.7-9%, 4.8-9%, 4.9-9%, 5-9%, 5.1-9%, 5.2-9%, 5.3-9%, 5.4-9%, 5.5-9%, 5.6-9%, 5.7-9%, 5.8-9%, 5.9-9%, 6-9%, 6.1-9%, 6.2-9%, 6.3-9%, 6.4-9%, 6.5-9%, 6.6-9%, 6.7-9%, 6.8-9%, 6.9-9%, 7-9%, 7.1-9%, 7.2-9%, 7.3-9%, 7.4-9%, 7.5-9%, 7.6-9%, 7.7-9%, 7.8-9%, 7.9-9%, 8-9%, 8.1-9%, 8.2-9%, 8.3-9%, 8.4-9%, 8.5-9%, 8.6-9%, 8.7-9%, 8.8-9%, 8.9-9%, 0-9.5%, 0.1-9.5%, 0.2-9.5%, 0.3-9.5%, 0.4-9.5%, 0.5-9.5%, 0.6-9.5%, 0.7-9.5%, 0.8-9.5%, 0.9-9.5%, 1-9.5%, 1.1-9.5%, 1.2-9.5%, 1.3-9.5%, 1.4-9.5%, 1.5-9.5%, 1.6-9.5%, 1.7-9.5%, 1.8-9.5%, 1.9-9.5%, 2-9.5%, 2.1-9.5%, 2.2-9.5%, 2.3-9.5%, 2.4-9.5%, 2.5-9.5%, 2.6-9.5%, 2.7-9.5%, 2.8-9.5%, 2.9-9.5%, 3-9.5%, 3.1-9.5%, 3.2-9.5%, 3.3-9.5%, 3.4-9.5%, 3.5-9.5%, 3.6-9.5%, 3.7-9.5%, 3.8-9.5%, 3.9-9.5%, 4-9.5%, 4.1-9.5%, 4.2-9.5%, 4.3-9.5%, 4.4-9.5%, 4.5-9.5%, 4.6-9.5%, 4.7-9.5%, 4.8-9.5%, 4.9-9.5%, 5-9.5%, 5.1-9.5%, 5.2-9.5%, 5.3-9.5%, 5.4-9.5%, 5.5-9.5%, 5.6-9.5%, 5.7-9.5%, 5.8-9.5%, 5.9-9.5%, 6-9.5%, 6.1-9.5%, 6.2-9.5%, 6.3-9.5%, 6.4-9.5%, 6.5-9.5%, 6.6-9.5%, 6.7-9.5%, 6.8-9.5%, 6.9-9.5%, 7-9.5%, 7.1-9.5%, 7.2-9.5%, 7.3-9.5%, 7.4-9.5%, 7.5-9.5%, 7.6-9.5%, 7.7-9.5%, 7.8-9.5%, 7.9-9.5%, 8-9.5%, 8.1-9.5%, 8.2-9.5%, 8.3-9.5%, 8.4-9.5%, 8.5-9.5%, 8.6-9.5%, 8.7-9.5%, 8.8-9.5%, 8.9-9.5%, 9-9.5%, 9.1-9.5%, 9.2-9.5%, 9.3-9.5%, 9.4-9.5%, 0-10%, 0.1-10%, 0.2-10%, 0.3-10%, 0.4-10%, 0.5-10%, 0.6-10%, 0.7-10%, 0.8-10%, 0.9-10%, 1-10%, 1.1-10%, 1.2-10%, 1.3-10%, 1.4-10%, 1.5-10%, 1.6-10%, 1.7-10%, 1.8-10%, 1.9-10%, 2-10%, 2.1-10%, 2.2-10%, 2.3-10%, 2.4-10%, 2.5-10%, 2.6-10%, 2.7-10%, 2.8-10%, 2.9-10%. 3-10%, 3.1-10%, 3.2-10%, 3.3-10%, 3.4-10%, 3.5-10%, 3.6-10%, 3.7-10%, 3.8-10%, 3.9-10%, 4-10%, 4.1-10%, 4.2-10%, 4.3-10%, 4.4-10%, 4.5-10%, 4.6-10%, 4.7-10%, 4.8-10%, 4.9-10%, 5-10%, 5.1-10%, 5.2-10%, 5.3-10%, 5.4-10%, 5.5-10%, 5.6-10%, 5.7-10%, 5.8-10%, 5.9-10%, 6-10%, 6.1-10%, 6.2-10%, 6.3-10%, 6.4-10%, 6.5-10%, 6.6-10%, 6.7-10%, 6.8-10%, 6.9-10%, 7-10%, 7.1-10%, 7.2-10%, 7.3-10%, 7.4-10%, 7.5-10%, 7.6-10%, 7.7-10%, 7.8-10%, 7.9-10%, 8-10%, 8.1-10%, 8.2-10%, 8.3-10%, 8.4-10%, 8.5-10%, 8.6-10%, 8.7-10%, 8.8-10%, 8.9-10%, 9-10%, 9.1-10%, 9.2-10%, 9.3-10%, 9.4-10%, 9.5-10%, 9.6-10%, 9.7-10%, 9.8-10%, or 9.9-10% w/v.

In certain embodiments, the formulation may include 0-10% w/v of sucrose.

In certain embodiments, the formulation may include 0-9% w/v of sucrose.

In certain embodiments, the formulation may include 0-8% w/v of sucrose.

In certain embodiments, the formulation may include 0-7% w/v of sucrose.

In certain embodiments, the formulation may include 0-6% w/v of sucrose.

In certain embodiments, the formulation may include 0-5% w/v of sucrose.

In certain embodiments, the formulation may include 0-4% w/v of sucrose.

In certain embodiments, the formulation may include 0-3% w/v of sucrose.

In certain embodiments, the formulation may include 0-2% w/v of sucrose.

In certain embodiments, the formulation may include 0-1% w/v of sucrose.

In certain embodiments, the formulation may include 1% w/v of sucrose.

In certain embodiments, the formulation may include 2% w/v of sucrose.

In certain embodiments, the formulation may include 3% w/v of sucrose.

In certain embodiments, the formulation may include 4% w/v of sucrose.

In certain embodiments, the formulation may include 5% w/v of sucrose.

In certain embodiments, the formulation may include 6% w/v of sucrose.

In certain embodiments, the formulation may include 7% w/v of sucrose.

In certain embodiments, the formulation may include 8% w/v of sucrose.

In certain embodiments, the formulation may include 9% w/v of sucrose.

In certain embodiments, the formulation may include 10% w/v of sucrose.

### Trehalose

In certain embodiments, the formulation may include at least one sugar which is disaccharide such as, but not limited to, trehalose.

In certain embodiments, the formulation may include trehalose at 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%. 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, or 10% w/v.

In certain embodiments, the formulation may include trehalose in a range of 0-1%, 0.1-1%, 0.2-1%, 0.3-1%, 0.4-1%, 0.5-1%, 0.6-1%, 0.7-1%, 0.8-1%, 0.9-1%, 0-1.5%, 0.1-1.5%, 0.2-1.5%, 0.3-1.5%, 0.4-1.5%, 0.5-1.5%, 0.6-1.5%, 0.7-1.5%, 0.8-1.5%, 0.9-1.5%, 1-1.5%, 1.1-1.5%, 1.2-1.5%, 1.3-1.5%, 1.4-1.5%, 0-2%, 0.1-2%, 0.2-2%, 0.3-2%, 0.4-2%, 0.5-2%, 0.6-2%, 0.7-2%, 0.8-2%, 0.9-2%, 1-2%, 1.1-2%, 1.2-2%, 1.3-2%, 1.4-2%, 1.5-2%, 1.6-2%, 1.7-2%, 1.8-2%, 1.9-2%, 0-2.5%, 0.1-2.5%, 0.2-2.5%, 0.3-2.5%, 0.4-2.5%, 0.5-2.5%, 0.6-2.5%, 0.7-2.5%, 0.8-2.5%, 0.9-2.5%, 1-2.5%, 1.1-2.5%, 1.2-2.5%, 1.3-2.5%, 1.4-2.5%, 1.5-2.5%, 1.6-2.5%, 1.7-2.5%, 1.8-2.5%, 1.9-2.5%, 2-2.5%, 2.1-2.5%, 2.2-2.5%, 2.3-2.5%, 2.4-2.5%, 0-3%, 0.1-3%, 0.2-3%, 0.3-3%, 0.4-3%, 0.5-3%, 0.6-3%, 0.7-3%, 0.8-3%, 0.9-3%, 1-3%, 1.1-3%, 1.2-3%, 1.3-3%, 1.4-3%, 1.5-3%, 1.6-3%, 1.7-3%, 1.8-3%, 1.9-3%, 2-3%. 2.1-3%, 2.2-3%, 2.3-3%, 2.4-3%, 2.5-3%, 2.6-3%, 2.7-3%, 2.8-3%, 2.9-3%, 0-3.5%, 0.1-3.5%, 0.2-3.5%, 0.3-3.5%, 0.4-3.5%, 0.5-3.5%, 0.6-3.5%, 0.7-3.5%, 0.8-3.5%, 0.9-3.5%, 1-3.5%, 1.1-3.5%, 1.2-3.5%, 1.3-3.5%, 1.4-3.5%, 1.5-3.5%, 1.6-3.5%, 1.7-3.5%, 1.8-3.5%, 1.9-3.5%, 2-3.5%, 2.1-3.5%, 2.2-3.5%, 2.3-3.5%, 2.4-3.5%, 2.5-3.5%, 2.6-3.5%, 2.7-3.5%, 2.8-3.5%, 2.9-3.5%, 3-3.5%, 3.1-3.5%, 3.2-3.5%, 3.3-3.5%, 3.4-3.5%, 0-4%, 0.1-4%, 0.2-4%, 0.3-4%, 0.4-4%, 0.5-4%, 0.6-4%, 0.7-4%, 0.8-4%, 0.9-4%, 1-4%, 1.1-4%, 1.2-4%, 1.3-4%, 1.4-4%, 1.5-4%, 1.6-4%, 1.7-4%, 1.8-4%, 1.9-4%, 2-4%, 2.1-4%, 2.2-4%, 2.3-4%, 2.4-4%, 2.5-4%, 2.6-4%, 2.7-4%, 2.8-4%, 2.9-4%, 3-4%, 3.1-4%, 3.2-4%, 3.3-4%, 3.4-4%, 3.5-4%, 3.6-4%, 3.7-4%, 3.8-4%, 3.9-4%, 0-4.5%, 0.1-4.5%, 0.2-4.5%, 0.3-4.5%, 0.4-4.5%, 0.5-4.5%, 0.6-4.5%, 0.7-4.5%, 0.8-4.5%, 0.9-4.5%, 1-4.5%, 1.1-4.5%, 1.2-4.5%, 1.3-4.5%, 1.4-4.5%, 1.5-4.5%, 1.6-4.5%, 1.7-4.5%, 1.8-4.5%, 1.9-4.5%, 2-4.5%, 2.1-4.5%, 2.2-4.5%, 2.3-4.5%, 2.4-4.5%, 2.5-4.5%, 2.6-4.5%, 2.7-4.5%, 2.8-4.5%, 2.9-4.5%, 3-4.5%, 3.1-4.5%, 3.2-4.5%, 3.3-4.5%, 3.4-4.5%, 3.5-4.5%, 3.6-4.5%, 3.7-4.5%, 3.8-4.5%, 3.9-4.5%, 4-4.5%, 4.1-4.5%, 4.2-4.5%, 4.3-4.5%, 4.4-4.5%, 0-5%, 0.1-5%, 0.2-5%, 0.3-5%, 0.4-5%, 0.5-5%, 0.6-5%, 0.7-5%, 0.8-5%, 0.9-5%, 1-5%, 1.1-5%, 1.2-5%, 1.3-5%, 1.4-5%, 1.5-5%, 1.6-5%, 1.7-5%, 1.8-5%, 1.9-5%, 2-5%, 2.1-5%, 2.2-5%, 2.3-5%, 2.4-5%, 2.5-5%, 2.6-5%, 2.7-5%, 2.8-5%, 2.9-5%, 3-5%, 3.1-5%, 3.2-5%, 3.3-5%, 3.4-5%, 3.5-5%, 3.6-5%, 3.7-5%, 3.8-5%, 3.9-5%, 4-5%, 4.1-5%, 4.2-5%, 4.3-5%, 4.4-5%, 4.5-5%, 4.6-5%, 4.7-5%, 4.8-5%, 4.9-5%, 0-5.5%, 0.1-5.5%, 0.2-5.5%, 0.3-5.5%, 0.4-5.5%, 0.5-5.5%, 0.6-5.5%, 0.7-5.5%, 0.8-5.5%, 0.9-5.5%, 1-5.5%, 1.1-5.5%, 1.2-5.5%, 1.3-5.5%, 1.4-5.5%, 1.5-5.5%, 1.6-5.5%, 1.7-5.5%, 1.8-5.5%, 1.9-5.5%, 2-5.5%, 2.1-5.5%, 2.2-5.5%, 2.3-5.5%, 2.4-5.5%, 2.5-5.5%, 2.6-5.5/0, 2.7-5.5%, 2.8-5.5%, 2.9-5.5%, 3-5.5%, 3.1-5.5%, 3.2-5.5%, 3.3-5.5%, 3.4-5.5%, 3.5-5.5%, 3.6-5.5%, 3.7-5.5%, 3.8-5.5%, 3.9-5.5%, 4-5.5%, 4.1-5.5%, 4.2-5.5%, 4.3-5.5%, 4.4-5.5%, 4.5-5.5%, 4.6-5.5%, 4.7-5.5%, 4.8-5.5%, 4.9-5.5%, 5-5.5%, 5.1-5.5%, 5.2-5.5%, 5.3-5.5%, 5.4-5.5%, 0-6%, 0.1-6%, 0.2-6%, 0.3-6%, 0.4-6%, 0.5-6%, 0.6-6%, 0.7-6%, 0.8-6%, 0.9-6%, 1-6%, 1.1-6%, 1.2-60%, 1.3-6%, 1.4-60%, 1.5-6%, 1.6-6%, 1.7-6%, 1.8-6%, 1.9-6%, 2-6%, 2.1-6%, 2.2-6%, 2.3-6%, 2.4-6%, 2.5-6%, 2.6-6%, 2.7-6%, 2.8-6%, 2.9-6%, 3-6%, 3.1-6%, 3.2-6%, 3.3-6%, 3.4-6%, 3.5-6%, 3.6-6%, 3.7-6%, 3.8-6%, 3.9-6%, 4-6%, 4.1-6%, 4.2-6%, 4.3-6%, 4.4-6%, 4.5-6%, 4.6-6%, 4.7-6%, 4.8-6%, 4.9-6%, 5-6%, 5.1-6%, 5.2-6%, 5.3-6%, 5.4-6%, 5.5-6%, 5.6-6%, 5.7-6%, 5.8-6%, 5.9-6%, 0-6.5%, 0.1-6.5%, 0.2-6.5%, 0.3-6.5%, 0.4-6.5%, 0.5-6.5%, 0.6-6.5%, 0.7-6.5%, 0.8-6.5%, 0.9-6.5%, 1-6.5%, 1.1-6.5%, 1.2-6.5%, 1.3-6.5%, 1.4-6.5%, 1.5-6.5%, 1.6-6.5%, 1.7-6.5%, 1.8-6.5%, 1.9-6.5%, 2-6.5%, 2.1-6.5%, 2.2-6.5%, 2.3-6.5%, 2.4-6.5%, 2.5-6.5%, 2.6-6.5%, 2.7-6.5%, 2.8-6.5%, 2.9-6.5%, 3-6.5%, 3.1-6.5%, 3.2-6.5%, 3.3-6.5%, 3.4-6.5%, 3.5-6.5%, 3.6-6.5%, 3.7-6.5%, 3.8-6.5%, 3.9-6.5%, 4-6.5%, 4.1-6.5%, 4.2-6.5%, 4.3-6.5%, 4.4-6.5%, 4.5-6.5%, 4.6-6.5%, 4.7-6.5%, 4.8-6.5%, 4.9-6.5%, 5-6.5%, 5.1-6.5%, 5.2-6.5%, 5.3-6.5%, 5.4-6.5%, 5.5-6.5%, 5.6-6.5%, 5.7-6.5%, 5.8-6.5%, 5.9-6.5%, 6-6.5%, 6.1-6.5%, 6.2-6.5%, 6.3-6.5%, 6.4-6.5%, 0-7%, 0.1-7%, 0.2-7%, 0.3-7%, 0.4-7%, 0.5-7%, 0.6-7%, 0.7-7%, 0.8-7%, 0.9-7%, 1-7%, 1.1-7%, 1.2-7%, 1.3-7%, 1.4-7%, 1.5-7%, 1.6-7%, 1.7-7%, 1.8-7%, 1.9-7%, 2-7%, 2.1-7%, 2.2-7%, 2.3-7%, 2.4-7%, 2.5-7%, 2.6-7%, 2.7-7%, 2.8-7%, 2.9-7%, 3-7%, 3.1-7%, 3.2-7%, 3.3-7%, 3.4-7%, 3.5-7%, 3.6-7%, 3.7-7%, 3.8-7%, 3.9-7%, 4-7%, 4.1-7%, 4.2-7%, 4.3-7%, 4.4-7%, 4.5-7%, 4.6-7%, 4.7-7%, 4.8-7%, 4.9-7%, 5-7%, 5.1-7%, 5.2-7%, 5.3-7%, 5.4-7%, 5.5-7%, 5.6-7%, 5.7-7%, 5.8-7%, 5.9-7%, 6-7%, 6.1-7%, 6.2-7%, 6.3-7%, 6.4-7%, 6.5-7%, 6.6-7%, 6.7-7%, 6.8-7%, 6.9-7%, 0-7.5%, 0.1-7.5%, 0.2-7.5%, 0.3-7.5%, 0.4-7.5%, 0.5-7.5%, 0.6-7.5%, 0.7-7.5%, 0.8-7.5%, 0.9-7.5%, 1-7.5%, 1.1-7.5%, 1.2-7.5%, 13-7.5%, 1.4-7.5%, 1.5-7.5%, 1.6-7.5%, 1.7-7.5%, 1.8-7.5%, 1.9-7.5%, 2-7.5%, 2.1-7.5%, 2.2-7.5%, 2.3-7.5%, 2.4-7.5%, 2.5-7.5%, 2.6-7.5%, 2.7-7.5%, 2.8-7.5%, 2.9-7.5%, 3-7.5%, 3.1-7.5%, 3.2-7.5%, 3.3-7.5%, 3.4-7.5%, 3.5-7.5%, 3.6-7.5%, 3.7-7.5%, 3.8-7.5%, 3.9-7.5%, 4-7.5%, 4.1-7.5%, 4.2-7.5%, 4.3-7.5%, 4.4-7.5%, 4.5-7.5%, 4.6-7.5%, 4.7-7.5%, 4.8-7.5%, 4.9-7.5%, 5-7.5%, 5.1-7.5%, 5.2-7.5%, 5.3-7.5%, 5.4-7.5%, 5.5-7.5%, 5.6-7.5%, 5.7-7.5%, 5.8-7.5%, 5.9-7.5%, 6-7.5%, 6.1-7.5%, 6.2-7.5%, 6.3-7.5%, 6.4-7.5%, 6.5-7.5%, 6.6-7.5%, 6.7-7.5%, 6.8-7.5%, 6.9-7.5%, 7-7.5%, 7.1-7.5%, 7.2-7.5%, 7.3-7.5%, 7.4-7.5%, 0-8%, 0.1-8%, 0.2-8%, 0.3-8%, 0.4-8%, 0.5-8%, 0.6-8%, 0.7-8%, 0.8-8%, 0.9-8%, 1-8%, 1.1-8%, 1.2-8%, 1.3-8%, 1.4-8%, 1.5-8%, 1.6-8%, 1.7-8%, 1.8-8%, 1.9-8%, 2-8%, 2.1-8%, 2.2-8%, 2.3-8%, 2.4-8%, 2.5-8%, 2.6-8%, 2.7-8%, 2.8-8%, 2.9-8%, 3-8%, 3.1-8%, 3.2-8%, 3.3-8%, 3.4-8%, 3.5-8%, 3.6-8%, 3.7-8%, 3.8-8%, 3.9-8%, 4-8%, 4.1-8%, 4.2-8%, 4.3-8%, 4.4-8%, 4.5-8%, 4.6-8%, 4.7-8%, 4.8-8%, 4.9-8%, 5-8%, 5.1-8%, 5.2-8%, 5.3-8%, 5.4-8%, 5.5-8%, 5.6-8%, 5.7-8%, 5.8-8%, 5.9-8%, 6-8%, 6.1-8%, 6.2-8%, 6.3-8%, 6.4-8%, 6.5-8%, 6.6-8%, 6.7-8%, 6.8-8%, 6.9-8%, 7-8%, 7.1-8%, 7.2-8%, 7.3-8%, 7.4-8%, 7.5-8%, 7.6-8%, 7.7-8%, 7.8-8%, 7.9-8%, 0-8.5%, 0.1-8.5%, 0.2-8.5%, 0.3-8.5%, 0.4-8.5%, 0.5-8.5%, 0.6-8.5%, 0.7-8.5%, 0.8-8.5%, 0.9-8.5%, 1-8.5%, 1.1-8.5%, 1.2-8.5%, 1.3-8.5%, 1.4-8.5%, 1.5-8.5%, 1.6-8.5%, 1.7-8.5%, 1.8-8.5%, 1.9-8.5%, 2-8.5%, 2.1-8.5%, 2.2-8.5%, 2.3-8.5%, 2.4-8.5%, 2.5-8.5%, 2.6-8.5%, 2.7-8.5%, 2.8-8.5%, 2.9-8.5%, 3-8.5%, 3.1-8.5%, 3.2-8.5%, 3.3-8.5%, 3.4-8.5%, 3.5-8.5%, 3.6-8.5%, 3.7-8.5%, 3.8-8.5%, 3.9-8.5%, 4-8.5%, 4.1-8.5%, 4.2-8.5%, 4.3-8.5%, 4.4-8.5%, 4.5-8.5%, 4.6-8.5%, 4.7-8.5%, 4.8-8.5%, 4.9-8.5%, 5-8.5%, 5.1-8.5%, 5.2-8.5%, 5.3-8.5%, 5.4-8.5%, 5.5-8.5%, 5.6-8.5%, 5.7-8.5%, 5.8-8.5%, 5.9-8.5%, 6-8.5%, 6.1-8.5%, 6.2-8.5%, 6.3-8.5%, 6.4-8.5%, 6.5-8.5%, 6.6-8.5%, 6.7-8.5%, 6.8-8.5%, 6.9-8.5%, 7-8.5%, 7.1-8.5%, 7.2-8.5%, 7.3-8.5%, 7.4-8.5%, 7.5-8.5%, 7.6-8.5%, 7.7-8.5%, 7.8-8.5%, 7.9-8.5%, 8-8.5%, 8.1-8.5%, 8.2-8.5%, 8.3-8.5%, 8.4-8.5%, 0-9%, 0.1-9%, 0.2-9%, 0.3-9%, 0.4-9%, 0.5-9%, 0.6-9%, 0.7-9%, 0.8-9%, 0.9-9%, 1-9%, 1.1-9%, 1.2-9%, 1.3-9%, 1.4-9%, 1.5-9%, 1.6-9%, 1.7-9%, 1.8-9%, 1.9-9%, 2-9%, 2.1-9%, 2.2-9%, 2.3-9%, 2.4-9%, 2.5-9%, 2.6-9%, 2.7-9%, 2.8-9%, 2.9-9%, 3-9%, 3.1-9%, 3.2-9%, 3.3-9%, 3.4-9%, 3.5-9%, 3.6-9%, 3.7-9%, 3.8-9%, 3.9-9%, 4-9%, 4.1-9%, 4.2-9%, 4.3-9%, 4.4-9%, 4.5-9%, 4.6-9%, 4.7-9%, 4.8-9%, 4.9-9%, 5-9%, 5.1-9%, 5.2-9%, 5.3-9%, 5.4-9%, 5.5-9%, 5.6-9%, 5.7-9%, 5.8-9%, 5.9-9%, 6-9%, 6.1-9%, 6.2-9%, 6.3-9%, 6.4-9%, 6.5-9%, 6.6-9%, 6.7-9%, 6.8-9%, 6.9-9%, 7-9%, 7.1-9%, 7.2-9%, 7.3-9%, 7.4-9%, 7.5-9%, 7.6-9%, 7.7-9%, 7.8-9%, 7.9-9%, 8-9%, 8.1-9%, 8.2-9%, 8.3-9%, 8.4-9%, 8.5-9%, 8.6-9%, 8.7-9%, 8.8-9%, 8.9-9%, 0-9.5%, 0.1-9.5%, 0.2-9.5%, 0.3-9.5%, 0.4-9.5%, 0.5-9.5%, 0.6-9.5%, 0.7-9.5%, 0.8-9.5%, 0.9-9.5%, 1-9.5%, 1.1-9.5%, 1.2-9.5%, 1.3-9.5%, 1.4-9.5%, 1.5-9.5%, 1.6-9.5%, 1.7-9.5%, 1.8-9.5%, 1.9-9.5%, 2-9.5%, 2.1-9.5%, 2.2-9.5%, 2.3-9.5%, 2.4-9.5%, 2.5-9.5%, 2.6-9.5%, 2.7-9.5%, 2.8-9.5%, 2.9-9.5%, 3-9.5%, 3.1-9.5%, 3.2-9.5%, 3.3-9.5%, 3.4-9.5%, 3.5-9.5%, 3.6-9.5%, 3.7-9.5%, 3.8-9.5%, 3.9-9.5%, 4-9.5%, 4.1-9.5%, 4.2-9.5%, 4.3-9.5%, 4.4-9.5%, 4.5-9.5%, 4.6-9.5%, 4.7-9.5%, 4.8-9.5%, 4.9-9.5%, 5-9.5%, 5.1-9.5%, 5.2-9.5%, 5.3-9.5%, 5.4-9.5%, 5.5-9.5%, 5.6-9.5%, 5.7-9.5%, 5.8-9.5%, 5.9-9.5%, 6-9.5%, 6.1-9.5%, 6.2-9.5%, 6.3-9.5%, 6.4-9.5%, 6.5-9.5%, 6.6-9.5%, 6.7-9.5%, 6.8-9.5%, 6.9-9.5%, 7-9.5%, 7.1-9.5%, 7.2-9.5%, 7.3-9.5%, 7.4-9.5%, 7.5-9.5%, 7.6-9.5%, 7.7-9.5%, 7.8-9.5%, 7.9-9.5%, 8-9.5%, 8.1-9.5%, 8.2-9.5%, 8.3-9.5%, 8.4-9.5%, 8.5-9.5%, 8.6-9.5%, 8.7-9.5%, 8.8-9.5%, 8.9-9.5%, 9-9.5%, 9.1-9.5%, 9.2-9.5%, 9.3-9.5%, 9.4-9.5%, 0-10%, 0.1-10%, 0.2-10%, 0.3-10%, 0.4-10%, 0.5-10%, 0.6-10%, 0.7-10%, 0.8-10%, 0.9-10%, 1-10%, 1.1-10%, 1.2-10%, 1.3-10%, 1.4-10%, 1.5-10%, 1.6-10%, 1.7-10%, 1.8-10%, 1.9-10%, 2-10%, 2.1-10%, 2.2-10%, 2.3-10%, 2.4-10%, 2.5-10%, 2.6-10%, 2.7-10%,2.8-10%, 2.9-10%, 3-10%, 3.1-10%, 3.2-10%, 3.3-10%, 3.4-10%, 3.5-10%, 3.6-10%, 3.7-10%, 3.8-10%, 3.9-10%, 4-10%, 4.1-10%, 4.2-10%, 4.3-10%, 4.4-10%, 4.5-10%, 4.6-10%, 4.7-10%, 4.8-10%, 4.9-10%, 5-10%, 5.1-10%, 5.2-10%, 5.3-10%, 5.4-10%, 5.5-10%, 5.6-10%, 5.7-10%, 5.8-10%, 5.9-10%, 6-10%, 6.1-10%, 6.2-10%, 6.3-10%, 6.4-10%, 6.5-10%, 6.6-10%, 6.7-10%, 6.8-10%, 6.9-10%, 7-10%, 7.1-10%, 7.2-10%, 7.3-10%, 7.4-10%, 7.5-10%, 7.6-10%, 7.7-10%, 7.8-10%, 7.9-10%, 8-10%, 8.1-10%, 8.2-10%, 8.3-10%, 8.4-10%, 8.5-10%, 8.6-10%, 8.7-10%, 8.8-10%, 8.9-10%, 9-10%, 9.1-10%, 9.2-10%, 9.3-10%, 9.4-10%, 9.5-10%, 9.6-10%, 9.7-10%, 9.8-10%, or 9.9-10% w/v.

In certain embodiments, the formulation may include 0-10% w/v of trehalose.

In certain embodiments, the formulation may include 0-9% w/v of trehalose.

In certain embodiments, the formulation may include 0-8% w/v of trehalose.

In certain embodiments, the formulation may include 0-7% w/v of trehalose.

In certain embodiments, the formulation may include 0-6% w/v of trehalose.

In certain embodiments, the formulation may include 0-5% w/v of trehalose.

In certain embodiments, the formulation may include 0-4% w/v of trehalose.

In certain embodiments, the formulation may include 0-3% w/v of trehalose.

In certain embodiments, the formulation may include 0-2% w/v of trehalose.

In certain embodiments, the formulation may include 0-1% w/v of trehalose.

In certain embodiments, the formulation may include 1% w/v of trehalose.

In certain embodiments, the formulation may include 2% w/v of trehalose.

In certain embodiments, the formulation may include 3% w/v of trehalose.

In certain embodiments, the formulation may include 4% w/v of trehalose.

In certain embodiments, the formulation may include 5% w/v of trehalose.

In certain embodiments, the formulation may include 6% w/v of trehalose.

In certain embodiments, the formulation may include 7% w/v of trehalose.

In certain embodiments, the formulation may include 8% w/v of trehalose.

In certain embodiments, the formulation may include 9% w/v of trehalose.

In certain embodiments, the formulation may include 10% w/v of trehalose.

### Sorbitol

In certain embodiments, the formulation may include at least one sugar substitute (e.g., a sugar alcohol) which is sorbitol.

In certain embodiments, the formulation may include sorbitol at 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%. 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%. 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, or 10% w/v.

In certain embodiments, the formulation may include sorbitol in a range of 0-1%, 0.1-1%, 0.2-1%, 0.3-1%, 0.4-1%, 0.5-1%, 0.6-1%, 0.7-1%, 0.8-1%, 0.9-1%, 0-1.5%, 0.1-1.5%, 0.2-1.5%, 0.3-1.5%, 0.4-1.5%, 0.5-1.5%, 0.6-1.5%, 0.7-1.5%, 0.8-1.5%, 0.9-1.5%, 1-1.5%, 1.1-1.5%, 1.2-1.5%, 1.3-1.5%, 1.4-1.5%, 0-2%, 0.1-2%, 0.2-2%, 0.3-2%, 0.4-2%, 0.5-2%, 0.6-2%, 0.7-2%, 0.8-2%, 0.9-2%, 1-2%, 1.1-2%, 1.2-2%, 1.3-2%, 1.4-2%, 1.5-2%, 1.6-2%, 1.7-2%, 1.8-2%, 1.9-2%, 0-2.5%, 0.1-2.5%, 0.2-2.5%, 0.3-2.5/0, 0.4-2.5%, 0.5-2.5%, 0.6-2.5%, 0.7-2.5%, 0.8-2.5%, 0.9-2.5%, 1-2.5%, 1.1-2.5%, 1.2-2.5%, 1.3-2.5%, 1.4-2.5%, 1.5-2.5%, 1.6-2.5%, 1.7-2.5%, 1.8-2.5%, 1.9-2.5%, 2-2.5%, 2.1-2.5%, 2.2-2.5%, 2.3-2.5%, 2.4-2.5%, 0-3%, 0.1-3%, 0.2-3%, 0.3-3%, 0.4-3%, 0.5-3%, 0.6-3%, 0.7-3%, 0.8-3%, 0.9-3%, 1-3%, 1.1-3%, 1.2-3%, 1.3-3%, 1.4-3%, 1.5-3%, 1.6-3%, 1.7-3%, 1.8-3%, 1.9-3%, 2-3%, 2.1-3%, 2.2-3%, 2.3-3%, 2.4-3%, 2.5-3%, 2.6-3%, 2.7-3%, 2.8-3%, 2.9-3%, 0-3.5%, 0.1-3.5%, 0.2-3.5%, 0.3-3.5%, 0.4-3.5%, 0.5-3.5%, 0.6-3.5%, 0.7-3.5%, 0.8-3.5%, 0.9-3.5%, 1-3.5%, 1.1-3.5%, 1.2-3.5%, 1.3-3.5%, 1.4-3.5%, 1.5-3.5%, 1.6-3.5%, 1.7-3.5%, 1.8-3.5%, 1.9-3.5%, 2-3.5%, 2.1-3.5%, 2.2-3.5%, 2.3-3.5%, 2.4-3.5%, 2.5-3.5%, 2.6-3.5%, 2.7-3.5%, 2.8-3.5%, 2.9-3.5%, 3-3.5%, 3.1-3.5%, 3.2-3.5%, 3.3-3.5%, 3.4-3.5%, 0-4%, 0.1-4%, 0.2-4%, 0.3-4%, 0.4-4%, 0.5-4%, 0.6-4%, 0.7-4%, 0.8-4%, 0.9-4%, 1-4%, 1.1-4%, 1.2-4%, 1.3-4%, 1.4-4%, 1.5-4%, 1.6-4%, 1.7-4%, 1.8-4%, 1.9-4%, 2-4%, 2.1-4%, 2.2-4%, 2.3-4%, 2.4-4%, 2.5-4%, 2.6-4%, 2.7-4%, 2.8-4%, 2.9-4%, 3-4%, 3.1-4%, 3.2-4%, 3.3-4%, 3.4-4%, 3.5-4%, 3.6-4%, 3.7-4%, 3.8-4%, 3.9-4%, 0-4.5%, 0.1-4.5%, 0.2-4.5%, 0.3-4.5%, 0.4-4.5%, 0.5-4.5%, 0.6-4.5%, 0.7-4.5%, 0.8-4.5%, 0.9-4.5%, 1-4.5%, 1.1-4.5%, 1.2-4.5%, 1.3-4.5%, 1.4-4.5%, 1.5-4.5%, 1.6-4.5%, 1.7-4.5%, 1.8-4.5%, 1.9-4.5%, 2-4.5%, 2.1-4.5%, 2.2-4.5%, 2.3-4.5%, 2.4-4.5%, 2.5-4.5%, 2.6-4.5%, 2.7-4.5%, 2.8-4.5%, 2.9-4.5%, 3-4.5%, 3.1-4.5%, 3.2-4.5%, 3.3-4.5%, 3.4-4.5%, 3.5-4.5%, 3.6-4.5%, 3.7-4.5%, 3.8-4.5%, 3.9-4.5%, 4-4.5%, 4.1-4.5%, 4.2-4.5%, 4.3-4.5%, 4.4-4.5%, 0-5%, 0.1-5%, 0.2-5%, 0.3-5%, 0.4-5%, 0.5-5%, 0.6-5%, 0.7-5%, 0.8-5/a, 0.9-5%, 1-5%, 1.1-5%, 1.2-5%, 1.3-5%, 1.4-5%, 1.5-5%, 1.6-5%, 1.7-5%, 1.8-5%, 1.9-5%, 2-5%, 2.1-5%, 2.2-5%, 2.3-5%, 2.4-5%, 2.5-5%, 2.6-5%, 2.7-5%, 2.8-5%, 2.9-5%, 3-5%, 3.1-5%, 3.2-5%, 3.3-5%, 3.4-5%, 3.5-5%, 3.6-5%, 3.7-5%, 3.8-5%, 3.9-5%, 4-5%, 4.1-5%, 4.2-5%, 4.3-5%, 4.4-5%, 4.5-5%, 4.6-5%, 4.7-5%, 4.8-5%, 4.9-5%, 0-5.5%, 0.1-5.5%, 0.2-5.5%, 0.3-5.5%, 0.4-5.5%, 0.5-5.5%, 0.6-5.5%, 0.7-5.5%, 0.8-5.5%, 0.9-5.5%, 1-5.5%, 1.1-5.5%, 1.2-5.5%, 1.3-5.5%, 1.4-5.5%, 1.5-5.5%, 1.6-5.5%, 1.7-5.5%, 1.8-5.5%, 1.9-5.5%, 2-5.5%, 2.1-5.5%, 2.2-5.5%, 2.3-5.5%, 2.4-5.5%, 2.5-5.5%, 2.6-5.5%, 2.7-5.5%, 2.8-5.5%, 2.9-5.5%, 3-5.5%, 3.1-5.5%, 3.2-5.5%, 3.3-5.5%, 3.4-5.5%, 3.5-5.5%, 3.6-5.5%, 3.7-5.5%, 3.8-5.5%, 3.9-5.5%, 4-5.5%, 4.1-5.5%, 4.2-5.5%, 4.3-5.5%, 4.4-5.5%, 4.5-5.5%, 4.6-5.5%, 4.7-5.5%, 4.8-5.5%, 4.9-5.5%, 5-5.5%, 5.1-5.5%, 5.2-5.5%, 5.3-5.5%, 5.4-5.5%, 0-6%, 0.1-6%, 0.2-6%, 0.3-6%, 0.4-6%, 0.5-6%, 0.6-6%, 0.7-6%, 0.8-6%, 0.9-6%, 1-6%, 1.1-6%, 1.2-6%, 1.3-6%, 1.4-6%, 1.5-6% a, 1.6-6%, 1.7-6%, 1.8-6%, 1.9-6%, 2-6%, 2.1-6%, 2.2-6%, 2.3-6%, 2.4-6%, 2.5-6%, 2.6-6%, 2.7-6%, 2.8-6%, 2.9-6%, 3-6%, 3.1-6%, 3.2-6%, 3.3-6%, 3.4-6%, 3.5-6%, 3.6-6%, 3.7-6%, 3.8-6%, 3.9-6%, 4-6%, 4.1-6%, 4.2-6%, 4.3-6%, 4.4-6%, 4.5-6%, 4.6-6%, 4.7-6%, 4.8-6%, 4.9-6%, 5-6%, 5.1-6%, 5.2-6%, 5.3-6%, 5.4-6%, 5.5-6%, 5.6-6%, 5.7-6%, 5.8-6%, 5.9-6%, 0-6.5%, 0.1-6.5%, 0.2-6.5%, 0.3-6.5%, 0.4-6.5%, 0.5-6.5%, 0.6-6.5%, 0.7-6.5%, 0.8-6.5%, 0.9-6.5%, 1-6.5%, 1.1-6.5%, 1.2-6.5%, 1.3-6.5%, 1.4-6.5%, 1.5-6.5%, 1.6-6.5%, 1.7-6.5%, 1.8-6.5%, 1.9-6.5%, 2-6.5%, 2.1-6.5/a, 2.2-6.5%, 2.3-6.5%, 2.4-6.5%, 2.5-6.5%, 2.6-6.5%, 2.7-6.5%, 2.8-6.5%, 2.9-6.5%, 3-6.5%, 3.1-6.5%, 3.2-6.5%, 3.3-6.5%, 3.4-6.5%, 3.5-6.5%, 3.6-6.5%, 3.7-6.5%, 3.8-6.5%, 3.9-6.5%, 4-6.5%, 4.1-6.5%, 4.2-6.5%, 4.3-6.5%, 4.4-6.5%, 4.5-6.5%, 4.6-6.5%, 4.7-6.5%, 4.8-6.5%, 4.9-6.5%, 5-6.5%, 5.1-6.5%, 5.2-6.5%, 5.3-6.5%, 5.4-6.5%, 5.5-6.5%, 5.6-6.5%, 5.7-6.5%, 5.8-6.5%, 5.9-6.5%, 6-6.5%, 6.1-6.5%, 6.2-6.5%, 6.3-6.5%, 6.4-6.5%, 0-7%, 0.1-7%, 0.2-7%, 0.3-7%, 0.4-7%, 0.5-7%, 0.6-7%, 0.7-7%, 0.8-7%, 0.9-7%, 1-7%, 1.1-7%, 1.2-7%,1.3-7%, 1.4-7%, 1.5-7%, 1.6-7%, 1.7-7%, 1.8-7%, 1.9-7%, 2-7%, 2.1-7%, 2.2-7%, 2.3-7%, 2.4-7%, 2.5-7%, 2.6-7%, 2.7-7%, 2.8-7%, 2.9-7%, 3-7%, 3.1-7%, 3.2-7%, 3.3-7%, 3.4-7%, 3.5-7%, 3.6-7%, 3.7-7%, 3.8-7%, 3.9-7%, 4-7%, 4.1-7%, 4.2-7%, 4.3-7%, 4.4-7%, 4.5-7%, 4.6-7%, 4.7-7%, 4.8-7%, 4.9-7%, 5-7%, 5.1-7%, 5.2-7%, 5.3-7%, 5.4-7%, 5.5-7%, 5.6-7%, 5.7-7%, 5.8-7%, 5.9-7%, 6-7%, 6.1-7%, 6.2-7%, 6.3-7%, 6.4-7%, 6.5-7%, 6.6-7%, 6.7-7%, 6.8-7%, 6.9-7%, 0-7.5%, 0.1-7.5%, 0.2-7.5%, 0.3-7.5%, 0.4-7.5%, 0.5-7.5%, 0.6-7.5%, 0.7-7.5%, 0.8-7.5%, 0.9-7.5%, 1-7.5%, 1.1-7.5%, 1.2-7.5%, 1.3-7.5%, 1.4-7.5%, 1.5-7.5%, 1.6-7.5%, 1.7-7.5%, 1.8-7.5%, 1.9-7.5%, 2-7.5%, 2.1-7.5%, 2.2-7.5%, 2.3-7.5%, 2.4-7.5%, 2.5-7.5%, 2.6-7.5%, 2.7-7.5%, 2.8-7.5%, 2.9-7.5%, 3-7.5%, 3.1-7.5%, 3.2-7.5%, 3.3-7.5%, 3.4-7.5%, 3.5-7.5%, 3.6-7.5%, 3.7-7.5%, 3.8-7.5%, 3.9-7.5%, 4-7.5%, 4.1-7.5%, 4.2-7.5%, 4.3-7.5%, 4.4-7.5%, 4.5-7.5%, 4.6-7.5%, 4.7-7.5%, 4.8-7.5%, 4.9-7.5%, 5-7.5%, 5.1-7.5%, 5.2-7.5%, 5.3-7.5%, 5.4-7.5%, 5.5-7.5%, 5.6-7.5%, 5.7-7.5%, 5.8-7.5%, 5.9-7.5%, 6-7.5%, 6.1-7.5%, 6.2-7.5%, 6.3-7.5%, 6.4-7.5%, 6.5-7.5%, 6.6-7.5%, 6.7-7.5%, 6.8-7.5%, 6.9-7.5%, 7-7.5%, 7.1-7.5%, 7.2-7.5%, 7.3-7.5%, 7.4-7.5%, 0-8%, 0.1-8%, 0.2-8%, 0.3-8%, 0.4-8%, 0.5-8%, 0.6-8%, 0.7-8%, 0.8-8%, 0.9-8%, 1-8%, 1.1-8%, 1.2-8%, 1.3-8%, 1.4-8%, 1.5-8%, 1.6-8%, 1.7-8%, 1.8-8%, 1.9-8%, 2-8%, 2.1-8%, 2.2-8%, 2.3-8%, 2.4-8%, 2.5-8%, 2.6-8%, 2.7-8%, 2.8-8%, 2.9-8%, 3-8%, 3.1-8%, 3.2-8%, 3.3-8%, 3.4-8%, 3.5-8%, 3.6-8%, 3.7-8%, 3.8-8%, 3.9-8%, 4-8%, 4.1-8%, 4.2-8%, 4.3-8%, 4.4-8%, 4.5-8%, 4.6-8%, 4.7-8%, 4.8-8%, 4.9-8%, 5-8%, 5.1-8%, 5.2-8%, 5.3-8%, 5.4-8%, 5.5-8%, 5.6-8%, 5.7-8%, 5.8-8%, 5.9-8%, 6-8%, 6.1-8%, 6.2-8%, 6.3-8%, 6.4-8%, 6.5-8%, 6.6-8%, 6.7-8%, 6.8-8%, 6.9-8%, 7-8%, 7.1-8%, 7.2-8%, 7.3-8%, 7.4-8%, 7.5-8%, 7.6-8%, 7.7-8%, 7.8-8%, 7.9-8%, 0-8.5%, 0.1-8.5%, 0.2-8.5%. 0.3-8.5%, 0.4-8.5%, 0.5-8.5%, 0.6-8.5%, 0.7-8.5%, 0.8-8.5%, 0.9-8.5%, 1-8.5%, 1.1-8.5%, 1.2-8.5%, 1.3-8.5%, 1.4-8.5%, 1.5-8.5%, 1.6-8.5%, 1.7-8.5%, 1.8-8.5%, 1.9-8.5%, 2-8.5%, 2.1-8.5%, 2.2-8.5%, 2.3-8.5%, 2.4-8.5%, 2.5-8.5%, 2.6-8.5%, 2.7-8.5%, 2.8-8.5%, 2.9-8.5%, 3-8.5%, 3.1-8.5%, 3.2-8.5%, 3.3-8.5%, 3.4-8.5%, 3.5-8.5%, 3.6-8.5%, 3.7-8.5%, 3.8-8.5%, 3.9-8.5%, 4-8.5%, 4.1-8.5%, 4.2-8.5%, 4.3-8.5%, 4.4-8.5%, 4.5-8.5%, 4.6-8.5%, 4.7-8.5%, 4.8-8.5%, 4.9-8.5%, 5-8.5%, 5.1-8.5%, 5.2-8.5%, 5.3-8.5%, 5.4-8.5%, 5.5-8.5%, 5.6-8.5%, 5.7-8.5%, 5.8-8.5%, 5.9-8.5%, 6-8.5%, 6.1-8.5%, 6.2-8.5%, 6.3-8.5%, 6.4-8.5%, 6.5-8.5%, 6.6-8.5%, 6.7-8.5%, 6.8-8.5%, 6.9-8.5%, 7-8.5%, 7.1-8.5%, 7.2-8.5%, 7.3-8.5%, 7.4-8.5%, 7.5-8.5%, 7.6-8.5%, 7.7-8.5%, 7.8-8.5%, 7.9-8.5%, 8-8.5%, 8.1-8.5%, 8.2-8.5%, 8.3-8.5%, 8.4-8.5%, 0-9%, 0.1-9%, 0.2-9%, 0.3-9%, 0.4-9%, 0.5-9%, 0.6-9%, 0.7-9%, 0.8-9%, 0.9-9%, 1-9%, 1.1-9%, 1.2-9%, 1.3-9%, 1.4-9%, 1.5-9%, 1.6-9%, 1.7-9%, 1.8-9%, 1.9-9%, 2-9%, 2.1-9%, 2.2-9%, 2.3-9%, 2.4-9%, 2.5-9%, 2.6-9%, 2.7-9%, 2.8-9%, 2.9-9%, 3-9%, 3.1-9%, 3.2-9%, 3.3-9%, 3.4-9%, 3.5-9%, 3.6-9%, 3.7-9%, 3.8-9%, 3.9-9%, 4-9%, 4.1-9%, 4.2-9%, 4.3-9%, 4.4-9%, 4.5-9%, 4.6-9%, 4.7-9%, 4.8-9%, 4.9-9%, 5-9%, 5.1-9%, 5.2-9%, 5.3-9%, 5.4-9%, 5.5-9%, 5.6-9%, 5.7-9%, 5.8-9%, 5.9-9%, 6-9%, 6.1-9%, 6.2-9%, 6.3-9%, 6.4-9%, 6.5-9%, 6.6-9%, 6.7-9%, 6.8-9%, 6.9-9%, 7-9%, 7.1-9%, 7.2-9%, 7.3-9%, 7.4-9%, 7.5-9%, 7.6-9%, 7.7-9%, 7.8-9%, 7.9-9%, 8-9%, 8.1-9%, 8.2-9%, 8.3-9%, 8.4-9%, 8.5-9%, 8.6-9%, 8.7-9%, 8.8-9%, 8.9-9%, 0-9.5%, 0.1-9.5%, 0.2-9.5%, 0.3-9.5%, 0.4-9.5%, 0.5-9.5%, 0.6-9.5%, 0.7-9.5%, 0.8-9.5%, 0.9-9.5%, 1-9.5%, 1.1-9.5%, 1.2-9.5%, 1.3-9.5%, 1.4-9.5%, 1.5-9.5%, 1.6-9.5%, 1.7-9.5%, 1.8-9.5%, 1.9-9.5%, 2-9.5%, 2.1-9.5%, 2.2-9.5%, 2.3-9.5%, 2.4-9.5%, 2.5-9.5%, 2.6-9.5%, 2.7-9.5%, 2.8-9.5%, 2.9-9.5%, 3-9.5%, 3.1-9.5%, 3.2-9.5%, 3.3-9.5%, 3.4-9.5%, 3.5-9.5%, 3.6-9.5%, 3.7-9.5%, 3.8-9.5%, 3.9-9.5%, 4-9.5%, 4.1-9.5%, 4.2-9.5%, 4.3-9.5%, 4.4-9.5%, 4.5-9.5%, 4.6-9.5%, 4.7-9.5%, 4.8-9.5%, 4.9-9.5%, 5-9.5%, 5.1-9.5%, 5.2-9.5%, 5.3-9.5%, 5.4-9.5%, 5.5-9.5%, 5.6-9.5%, 5.7-9.5%, 5.8-9.5%, 5.9-9.5%, 6-9.5%, 6.1-9.5%, 6.2-9.5%, 6.3-9.5%, 6.4-9.5%, 6.5-9.5%, 6.6-9.5%, 6.7-9.5%, 6.8-9.5%, 6.9-9.5%, 7-9.5%, 7.1-9.5%, 7.2-9.5%, 7.3-9.5%, 7.4-9.5%, 7.5-9.5%, 7.6-9.5%, 7.7-9.5%, 7.8-9.5%, 7.9-9.5%, 8-9.5%, 8.1-9.5%, 8.2-9.5%, 8.3-9.5%, 8.4-9.5%, 8.5-9.5%, 8.6-9.5%, 8.7-9.5%, 8.8-9.5%, 8.9-9.5%, 9-9.5%, 9.1-9.5%, 9.2-9.5%, 9.3-9.5%, 9.4-9.5%, 0-10%, 0.1-10%, 0.2-10%, 0.3-10%, 0.4-10%, 0.5-10%, 0.6-10%, 0.7-10%, 0.8-10%, 0.9-10%, 1-10%, 1.1-10%, 1.2-10%, 1.3-10%, 1.4-10%, 1.5-10%, 1.6-10%, 1.7-10%, 1.8-10%, 1.9-10%, 2-10%, 2.1-10%, 2.2-10%, 2.3-10%, 2.4-10%, 2.5-10%, 2.6-10%, 2.7-10%, 2.8-10%, 2.9-10%. 3-10%, 3.1-10%, 3.2-10%, 3.3-10%, 3.4-10%, 3.5-10%, 3.6-10%, 3.7-10%, 3.8-10%, 3.9-10%, 4-10%, 4.1-10%, 4.2-10%, 4.3-10%, 4.4-10%, 4.5-10%, 4.6-10%, 4.7-10%, 4.8-10%, 4.9-10%, 5-10%, 5.1-10%, 5.2-10%, 5.3-10%, 5.4-10%, 5.5-10%, 5.6-10%, 5.7-10%, 5.8-10%, 5.9-10%, 6-10%, 6.1-10%, 6.2-10%, 6.3-10%, 6.4-10%, 6.5-10%, 6.6-10%, 6.7-10%, 6.8-10%, 6.9-10%, 7-10%, 7.1-10%, 7.2-10%, 7.3-10%, 7.4-10%, 7.5-10%, 7.6-10%, 7.7-10%, 7.8-10%, 7.9-10%, 8-10%, 8.1-10%, 8.2-10%, 8.3-10%, 8.4-10%, 8.5-10%, 8.6-10%, 8.7-10%, 8.8-10%, 8.9-10%, 9-10%, 9.1-10%, 9.2-10%, 9.3-10%, 9.4-10%, 9.5-10%, 9.6-10%, 9.7-10%, 9.8-10%, or 9.9-10% w/v.

In certain embodiments, the formulation may include 0-10% w/v of sorbitol.

In certain embodiments, the formulation may include 0-9% w/v of sorbitol.

In certain embodiments, the formulation may include 0-8% w/v of sorbitol.

In certain embodiments, the formulation may include 0-7% w/v of sorbitol.

In certain embodiments, the formulation may include 0-6% w/v of sorbitol.

In certain embodiments, the formulation may include 0-5% w/v of sorbitol.

In certain embodiments, the formulation may include 0-4% w/v of sorbitol.

In certain embodiments, the formulation may include 0-3% w/v of sorbitol.

In certain embodiments, the formulation may include 0-2% w/v of sorbitol.

In certain embodiments, the formulation may include 0-1% w/v of sorbitol.

In certain embodiments, the formulation may include 1% w/v of sorbitol.

In certain embodiments, the formulation may include 2% w/v of sorbitol.

In certain embodiments, the formulation may include 3% w/v of sorbitol.

In certain embodiments, the formulation may include 4% w/v of sorbitol.

In certain embodiments, the formulation may include 5% w/v of sorbitol.

In certain embodiments, the formulation may include 6% w/v of sorbitol.

In certain embodiments, the formulation may include 7% w/v of sorbitol.

In certain embodiments, the formulation may include 8% w/v of sorbitol.

In certain embodiments, the formulation may include 9% w/v of sorbitol.

In certain embodiments, the formulation may include 10% w/v of sorbitol.

### Surfactant

In some embodiments, formulations of pharmaceutical compositions described herein may comprise a surfactant. Surfactants may help control shear forces in suspension cultures. Surfactants used herein may be anionic, zwitterionic, or non-ionic surfactants and may include those known in the art that are suitable for use in pharmaceutical formulations. Examples of anionic surfactants include, but are not limited to, sulfate, sulfonate, phosphate esters, and carboxylates. Examples of nonionic surfactants include, but are not limited to, ehoxylates, fatty alcohol ethoxylates, alkylphenol ethoxylates (e.g., nonoxynols, Triton X-100), fatty acid ethoxylates, ethoxylated amines and/or fatty acid amides (e.g., polyethoxylated tallow amine, cocamide monoethanolamine, cocamide diethanolamine), ethylene oxide/propylene oxide copolymer (e.g., Poloxamers such as Pluronic® F-68 or F-127), esters of fatty acids and polyhydric alcohols, fatty acid alkanolamides, ethoxylated aliphatic acids, ethoxylated aliphatic alcohols, ethoxylated sorbitol fatty acid esters, ethoxylated glycerides, ethoxylated block copolymers with EDTA (ethylene diaminetetraacetic acid), ethoxylated cyclic ether adducts, ethoxylated amide and imidazoline adducts, ethoxylated amine adducts, ethoxylated mercaptan adducts, ethoxylated condensates with alkyl phenols, ethoxylated nitrogen-based hydrophobes, ethoxylated polyoxypropylenes, polymeric silicones, fluorinated surfactants, and polymerizable surfactants. Examples of zwitterionic surfactants include, but are not limited to, alkylamido betaines and amine oxides thereof, alkyl betaines and amine oxides thereof, sulfo betaines, hydroxy sulfo betaines, amphoglycinates, amphopropionates, balanced amphopolycarboxyglycinates, and alkyl polyaminoglycinates. Proteins have the ability of being charged or uncharged depending on the pH; thus, at the right pH, a protein, preferably with a pI of about 8 to 9, such as modified Bovine Serum Albumin or chymotrypsinogen, could function as a zwitterionic surfactant. Various mixtures of surfactants can be used if desired.

### Copolymers

In certain embodiments, at least one of the components in the formulation is copolymer.

In certain embodiments, the formulation may include at least one copolymer at a concentration of 0.00001%, 0.0001%, 0.001%, 0.01%, 0.1%, or 1% w/v.

In certain embodiments, the formulation may include at least one copolymer in a range of 0.00001%-0.0001%, 0.00001%-0.001%, 0.00001%-0.01%, 0.00001%-0.1%, 0.00001%-1%, 0.0001%-0.001%, 0.0001%-0.01%, 0.0001%-0.1%, 0.0001%-1%, 0.001%-0.01%, 0.001%-0.1%, 0.001%-1%, 0.01%-0.1%, 0.01%-1%, or 0.1-1% w/v.

In certain embodiments, the formulation may include 0.001% w/v copolymer.

In certain embodiments, the copolymer is an ethylene oxide/propylene oxide copolymer.

In certain embodiments, the formulation may include at least one ethylene oxide/propylene oxide copolymer at a concentration of 0.00001%, 0.0001%, 0.001%, 0.01%, 0.1%, or 1% w/v.

In certain embodiments, the formulation may include at least one ethylene oxide/propylene oxide copolymer in a range of 0.00001%-0.0001%, 0.00001%-0.001%, 0.00001%-0.01%, 0.00001%-0.1%, 0.00001%-1%, 0.0001%-0.001%, 0.0001%-0.01%. 0.0001%-0.1%, 0.0001%-1%, 0.001%-0.01%, 0.001%-0.1%, 0.001%-1%, 0.01%-0.1%, 0.01%-1%, or 0.1-1% w/v.

In certain embodiments, the formulation may include 0.001% w/v ethylene oxide/propylene oxide copolymer.

In certain embodiments, the formulation may include at least one ethylene oxide/propylene copolymer which is a Poloxamer. In certain embodiments, the formulation may include Poloxamer at a concentration of 0.00001%, 0.0001%, 0.001%, 0.01%, 0.1%, or 1% w/v.

In certain embodiments, the formulation may include Poloxamer in a range of 0.00001%-0.0001%, 0.00001%-0.001%, 0.00001%-0.01%, 0.00001%-0.1%, 0.00001%-1%. 0.0001%-0.001%, 0.0001%-0.01%, 0.0001%-0.1%, 0.0001%-1%, 0.001%-0.01%, 0.001%-0.1%, 0.001%-1%, 0.01%-0.1%, 0.01%-1%, or 0.1-1% w/v.

In certain embodiments, the formulation may include 0.001% w/v Poloxamer.

In certain embodiments, the formulation may include at least one ethylene oxide/propylene copolymer which is Poloxamer 188 (e.g., Pluronic® F-68). In certain embodiments, the formulation may include Poloxamer 188 at a concentration of 0.00001%, 0.0001%, 0.001%, 0.01%, 0.1%, or 1%% w/v.

In certain embodiments, the formulation may include Poloxamer 188 in a range of 0.0001%-0.0001%, 0.00001%0.001%, 0.00001%-0.01%, 0.00001%-0.1%, 0.00001%-1%, 0.0001%-0.001%, 0.0001%-0.01%, 0.0001%-0.1%, 0.0001%-1%, 0.001%-0.01%, 0.001%-0.1%, 0.001%-1%, 0.01%-0.1%, 0.01%-1%, or 0.1-1% w/v.

In certain embodiments, the formulation may include 0.001%-0.1 w/v Poloxamer 188.

In certain embodiments, the formulation may include 0.001% w/v Poloxamer 188.

In certain embodiments, the formulation may include at least one ethylene oxide/propylene copolymer which is Pluronic® F-68. In certain embodiments, the formulation may include Pluronic® F-68 at a concentration of 0.00001%, 0.0001%, 0.001%, 0.01%, 0.1%, or 1% w/v.

In certain embodiments, the formulation may include Pluronic® F-68 in a range of 0.00001%-0.0001%, 0.00001%-0.001%, 0.00001%-0.01%, 0.00001%,0.1%, 0.00001%-1%, 0.0001%-0.001%, 0.0001%-0.01%, 0.000.%-0.1%, 0.0001%-1%, 0.001%-0.01%, 0.001%-0.1%, 0.001%-1%, 0.01%-0.1%, 0.01%-1%, or 0.1-1% w/v.

In certain embodiments, the formulation may include 0.001%0.1% w/v Pluronic® F-68.

### Formulation Properties

In certain embodiments, the formulation has been optimized to have a specific pH, osmolality, concentration, concentration of AAV particle, and/or total dose of AAV particle.

pH

In certain embodiments, the formulation may be optimized for a specific pH. In certain embodiments, the formulation may include a pH buffering agent (also referred to herein as “buffering agent”) which is a weak acid or base that, when used in the formulation, maintains the pH of the formulation near a chosen value even after another acid or base is added to the formulation. The pH of the formulation may be, but is not limited, to 0, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, and 14.

In certain embodiments, the formulation may be optimized for a specific pH range. The pH range may be, but is not limited to, 0-4, 1-5, 2-6, 3-7, 4-8, 5-9, 6-10, 7-11, 8-12, 9-13, 10-14, 0-1.5, 1-2.5, 2-3.5, 3-4.5, 4-5.5, 5-6.5, 6-7.5, 7-8.5, 8-9.5, 9-10.5, 10-11.5, 11-12.5, 12-13.5, 0-1, 1-2, 2-3, 3-4, 4-5, 5-6, 6-7, 7-8, 8-9, 9-10, 10-11, 11-12, 12-13, 13-14, 0-0.5, 0.5-1, 1-1.5, 1.5-2, 2-2.5, 2.5-3, 3-3.5, 3.5-4, 4-4.5, 4.5-5, 5-5.5, 5.5-6, 6-6.5, 6.5-7, 7-7.5, 7.2-8.2, 7.2-7.6, 7.3-7.7, 7.5-8, 7.8-8.2, 8-8.5, 8.5-9, 9-9.5, 9.5-10, 10-10.5, 10.5-11, 11-11.5, 11.5-12, 12-12.5, 12.5-13, 13-13.5, or 13.5-14.

In certain embodiments, the pH of the formulation is between 6 and 8.5.

In certain embodiments, the pH of the formulation is between 7 and 8.5

In certain embodiments, the pH of the formulation is between 7 and 7.6.

In certain embodiments, the pH of the formulation is 7.

In certain embodiments, the pH of the formulation is 7.1.

In certain embodiments, the pH of the formulation is 7.2.

In certain embodiments, the pH of the formulation is 7.3.

In certain embodiments, the pH of the formulation is 7.4.

In certain embodiments, the pH of the formulation is 7.5.

In certain embodiments, the pH of the formulation is 7.6.

In certain embodiments, the pH of the formulation is 7.7.

In certain embodiments, the pH of the formulation is 7.8.

In certain embodiments, the pH of the formulation is 7.9.

In certain embodiments, the pH of the formulation is 8.

In certain embodiments, the pH of the formulation is 8.1.

In certain embodiments, the pH of the formulation is 8.2.

In certain embodiments, the pH of the formulation is 8.3.

In certain embodiments, the pH of the formulation is 8.4.

In certain embodiments, the pH of the formulation is 8.5.

In certain embodiments, the pH is determined when the formulation is at 5° C.

In certain embodiments, the pH is determined when the formulation is at 25° C.

Suitable buffering agents may include, but not limited to, Tris HCl, Tris base, sodium phosphate (monosodium phosphate and/or disodium phosphate), potassium phosphate (monopotassium phosphate and/or dipotassium phosphate), histidine, boric acid, citric acid, glycine, HEPES (4-(2-hydroxyethyl)-1-piperazineethanesulfonic acid), and MOPS (3-(N-morpholino)propanesulfonic acid).

Concentration of buffering agents in the formulation may be between 1-50 mM, between 1-25 mM, between 5-30 mM, between 5-20 mM, between 5-15 mM, between 10-40 mM, or between 15-30 mM. Concentration of buffering agents in the formulation may be about 1 mM, 5 mM, 7.5 mM, 10 mM, 12.5 mM, 15 mM, 20 mM, 25 mM, 30 mM, 35 mM, 40 mM, or 50 mM.

In some embodiments, the formulation may include, but is not limited to, phosphate-buffered saline (PBS). As a non-limiting example, the PBS may include sodium chloride, potassium chloride, disodium phosphate, monopotassium phosphate, and distilled water. In some instances, the PBS does not contain potassium or magnesium. In other instances, the PBS contains calcium and magnesium.

In some embodiments, buffering agents used in the formulations of pharmaceutical compositions described herein may comprise sodium phosphate (monosodium phosphate and/or disodium phosphate). As a non-limiting example, sodium phosphate may be adjusted to a pH (at 5° C.) within the range of 7.4±0.2. In some embodiments, buffering agents used in the formulations of pharmaceutical compositions described herein may comprise Tris base. Tris base may be adjusted with hydrochloric acid to any pH within the range of 7.1 and 9.1. As a non-limiting example, Tris base used in the formulations described herein may be adjusted to 8.0±0.2. As a non-limiting example, Tris base used in the formulations described herein may be adjusted to 7.5±0.2.

### Osmolality

In certain embodiments, the formulation may be optimized for a specific osmolality. The osmolality of the formulation may be, but is not limited to, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 mOsm/kg (milliosmoles/kg).

In certain embodiments, the formulation may be optimized for a specific range of osmolality. The range may be, but is not limited to, 350-360, 360-370, 370-380, 380-390, 390-400, 400-410, 410-420, 420-430, 430-440, 440-450, 450-460, 460-470, 470-480, 480-490, 490-500, 350-370, 360-380, 370-390, 380-400, 390-410, 400-420, 410-430, 420-440, 430-450, 440-460, 450-470, 460-480, 470-490, 480-500, 350-375, 375-400, 400-425, 425-450, 450-475, 475-500, 350-380, 360-390, 370-400, 380-410, 390-420, 400-430, 410-440, 420-450, 430-460, 440-470, 450-480, 460-490, 470-500, 350-390, 360-400, 370-410, 380-420, 390-430, 400-440, 410-450, 420-460, 430-470, 440-480, 450-490, 460-500, 350-400, 360-410, 370-420, 380-430, 390-440, 400-450, 410-460, 420-470, 430-480, 440-490, 450-500, 350-410, 360-420, 370-430, 380-440, 390-450, 400-460, 410-470, 420-480, 430-490, 440-500, 350-420, 360-430, 370-440, 380-450, 390-460, 400-470, 410-480, 420-490, 430-500, 350-430, 360-440, 370-450, 380-460, 390-470, 400-480, 410-490, 420-500, 350-440, 360-450, 370-460, 380-470, 390-480, 400-490, 410-500, 350-450, 360-460, 370-470, 380-480, 390-490, 400-500, 350-460, 360-470, 370-480, 380-490, 390-500, 350-470, 360-480, 370-490, 380-500, 350-480, 360-490, 370-500, 350-490, 360-500, or 350-500 mOsm/kg.

In certain embodiments, the osmolality of the formulation is between 350-500 mOsm/kg.

In certain embodiments, the osmolality of the formulation is between 400-500 mOsm/kg

In certain embodiments, the osmolality of the formulation is between 400-480 mOsm/kg.

In certain embodiments, the osmolality is 395 mOsm/kg.

In certain embodiments, the osmolality is 413 mOsm/kg.

In certain embodiments, the osmolality is 420 mOsm/kg.

In certain embodiments, the osmolality is 432 mOsm/kg.

In certain embodiments, the osmolality is 447 mOsm/kg.

In certain embodiments, the osmolality is 450 mOsm/kg.

In certain embodiments, the osmolality is 452 mOsm/kg.

In certain embodiments, the osmolality is 459 mOsm/kg.

In certain embodiments, the osmolality is 472 mOsm/kg.

In certain embodiments, the osmolality is 490 mOsm/kg.

In certain embodiments, the osmolality is 496 mOsm/kg.

### Concentration of AAV Particle

In certain embodiments, the concentration of AAV particle in the formulation may be between about 1×10⁶ VG/ml and about 1×10¹⁶ VG/ml. As used herein, “VG/ml” represents vector genomes (VG) per milliliter (ml). VG/ml also may describe genome copy per milliliter or DNase resistant particle per milliliter.

In certain embodiments, the formulation may include an AAV particle concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁹, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹¹, 6×10¹⁰, 7×10¹⁰, 8×10¹⁰, 9×10¹⁰, 1×10¹⁰, 2×10¹⁰, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 5×10¹¹, 6×10¹¹, 7×10¹¹, 7.0×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10², 2.9×10¹², 3×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹² 7.1×10¹², 7.2×10¹², 7.3×10¹², 7.4×10¹², 7.5×10¹², 7.6×10¹², 7.7×10¹², 7.8×10¹², 7.9×10¹², 8×10¹², 8.1×10¹², 8.2×10¹², 8.3×10¹², 8.4×10, 8.5×10¹², 8.6×10¹², 8.7×10¹², 8.8×10¹², 8.9×10¹², 9×10¹², 1×10¹³, 1.1×10¹³, 1.2×10¹³, 1.3×10¹³, 1.4×10¹³, 1.5×10¹³, 1.6×10¹³, 1.7×10¹³, 1.8×10¹³, 1.9×10¹³, 2×10¹³, 2.1×10¹³, 2.2×10¹³, 2.3×10¹³, 2.4×10¹³, 2.5×10¹³, 2.6×10¹³, 2.7×10¹³, 2.8×10¹³, 2.9×10¹³, 3×10¹³, 3.1×10¹³, 3.2×10¹³, 3.3×10¹³, 3.4×10¹³, 3.5×10¹³, 3.6×10¹³, 3.7×10¹³, 3.8×10¹³, 3.9×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/ml.

In certain embodiments, the concentration of AAV particle in the formulation is between 1×10¹¹ and 5×10¹³, between 1×10¹² and 5×10¹², between 2×10¹² and 1×10¹³, between 5×10¹² and 1×10¹³, between 1×10¹³ and 2×10¹³, between 2×10¹³ and 3×10¹³, between 2×10¹³ and 2.5×10¹³, between 2.5×10¹³ and 3×10¹³, or no more than 5×10¹³ VG/ml.

In certain embodiments, the concentration of AAV particle in the formulation is 2.7×10¹¹ VG/ml.

In certain embodiments, the concentration of AAV particle in the formulation is 9×10¹¹ VG/ml.

In certain embodiments, the concentration of AAV particle in the formulation is 1.2×10¹² VG/ml.

In certain embodiments, the concentration of AAV particle in the formulation is 2.7×10¹² VG/ml.

In certain embodiments, the concentration of AAV particle in the formulation is 4×10¹² VG/ml.

In certain embodiments, the concentration of AAV particle in the formulation is 6×10¹² VG/ml.

In certain embodiments, the concentration of AAV particle in the formulation is 7.9×10¹² VG/ml.

In certain embodiments, the concentration of AAV particle in the formulation is 8×10¹² VG/ml.

In certain embodiments, the concentration of AAV particle in the formulation is 1×10¹³ VG/ml.

In certain embodiments, the concentration of AAV particle in the formulation is 1.8×10¹³ VG/m1.

In certain embodiments, the concentration of AAV particle in the formulation is 2.2×10¹³ VG/ml.

In certain embodiments, the concentration of AAV particle in the formulation is 2.7×10¹³ VG/ml.

In certain embodiments, the concentration of AAV particle in the formulation is 3.5×10¹³ VG/ml.

In certain embodiments, the concentration of AAV particle in the formulation is 2.7-3.5×10¹³ VG/ml.

In certain embodiments, the concentration of AAV particle in the formulation is 7.0×10¹³ VG/ml.

In certain embodiments, the concentration of AAV particle in the formulation is 5.0×10¹² VG/mL

In certain embodiments, the concentration of AAV particle in the formulation may be between about 1×10⁶ total capsid/mL and about 1×10¹⁶ total capsid/ml. In certain embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹⁰, 2×10¹¹, 3×10¹¹, 4×10¹¹, 5×10¹¹, 6×10¹¹, 7×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 0.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹², 3×10¹², 3.1×10¹², 3.2×10¹², 3.3×10¹², 3.4×10¹², 3.5×10¹², 3.6×10¹², 3.7×10¹², 3.8×10¹², 3.9×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 8×10¹², 9×10¹², 1×10¹³, 2×10¹³, 2.1×10¹³, 2.2×10¹³, 2.3×10¹³, 2.4×10¹³, 2.5×10¹³, 2.6×10¹³, 2.7×10¹³, 2.8×10¹³, 2.9×10¹³, 3×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ total capsid/ml.

### Total Dose of AAV Particle

In certain embodiments, the total dose of the AAV particle in the formulation may be between about 1×10⁶ VG and about 1×10¹⁶ VG. In certain embodiments, the formulation may include a total dose of AAV particle of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹² 2.9×10¹², 3×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 7.1×10¹², 7.2×10¹², 7.3×10¹², 7.4×10¹², 7.5×10¹², 7.6×10¹², 7.7×10¹², 7.8×10¹², 7.9×10¹², 8×10¹², 8.1×10¹², 8.2×10¹², 8.3×10¹², 8.4×10¹², 8.5×10¹², 8.6×10¹², 8.7×10¹², 8.8×10¹², 8.9×10¹², 9×10¹², 1×10¹³, 1.1×10¹³, 1.2×10¹³, 1.3×10¹³, 1.4×10¹³, 1.5×10¹³, 1.6×10¹³, 1.7×10¹³, 1.8×10¹³, 1.9×10¹³, 2×10¹³, 2.1×10¹³, 2.2×10¹³, 2.3×10¹³, 2.4×10¹³, 2.5×10¹³, 2.6×10¹³, 2.7×10¹³, 2.8×10¹³, 2.9×10¹³, 3×10¹³, 3.1×10¹³, 3.2×10¹³, 3.3×10¹³, 3.4×10¹³, 3.5×10¹³, 3.6×10¹³, 3.7×10¹³, 3.8×10¹³, 3.9×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG.

In certain embodiments, the total dose of AAV particle in the formulation is between 1×10¹¹ and 5×10¹³VG.

In certain embodiments, the total dose of AAV particle in the formulation is between 1×10¹¹ and 2×10¹⁴ VG.

In certain embodiments, the total dose of AAV particle in the formulation is 1.4×10¹¹ VG.

In certain embodiments, the total dose of AAV particle in the formulation is 4.5×10¹¹ VG.

In certain embodiments, the total dose of AAV particle in the formulation is 6.8×10¹¹ VG.

In certain embodiments, the total dose of AAV particle in the formulation is 1.4×10¹² VG.

In certain embodiments, the total dose of AAV particle in the formulation is 2.2×10¹² VG.

In certain embodiments, the total dose of AAV particle in the formulation is 4.6×10¹¹ VG.

In certain embodiments, the total dose of AAV particle in the formulation is 9.2×10¹² VG.

In certain embodiments, the total dose of AAV particle in the formulation is 1.0×10¹³ VG.

In certain embodiments, the total dose of AAV particle in the formulation is 2.3×10¹³ VG.

### Exemplary Formulations

Described below are exemplary, non-limiting formulations of the present disclosure. The formulations may include AAV-particle formulations. Table 2 presents a summary of the components and properties of certain exemplary formulations of the present disclosure. Each formulation may optionally include 0.001%-0.1% (w/v) of Poloxamer 188 (e.g., Pluronic F-68).

TABLE 2
Formulations

| | Sodium | Potassium | Sodium | Potassium | Sugar | | | |
|---|---|---|---|---|---|---|---|---|
| Formulation | phosphate | phosphate | chloride | chloride | | Other | | Osmolality |
| ID. | (mM) | (mM) | (mM) | (mM) | (w/v) | (mM) | pH | (mOsm/kg) |
| VYFORM1 | 10 | 1.5 | 95 | — | 7% (S) | — | 7.4 | — |
| VYFORM2 | 2.7 | 1.5 | 155 | — | 5% (S) | — | 7.2 | 450 |
| VYFORM3 | 2.7 | 1.5 | 107 | — | 7% (S) | — | 6.9 | 428 |
| VYFORM4 | 2.7 | 1.5 | 92 | — | 7% (S) | — | 6.9 | 402 |
| VYFORM5 | 2.7 | 1.5 | 98 | — | 9% (S) | — | 6.9 | 428 |
| VYFORM6 | 2.7 | 1.5 | 83 | — | 9% (S) | — | 6.9 | 402 |
| VYFORM7 | 2.7 | 1.5 | 150 | — | 7% (S) | — | — | — |
| VYFORM8 | 2.7 | 1.5 | 150 | — | 9% (S) | — | — | — |
| VYFORM9 | 10 | 2 | 192 | 2.7 | 1% (S) | — | 7.4 | — |
| VYFORM10 | 10 | 2 | 150 | 2.7 | 3% (S) | — | — | — |
| VYFORM11 | 10 | 2 | 125 | 2.7 | 5% (T) | — | — | — |
| VYF0RM12 | — | 2 | 125 | 2.7 | 5% | 10 (His) | — | — |
| VYFORM13 | — | — | 142 | 1.5 | 5% (S) | 10 (Tris) | 7.1 | 424 |
| VYFORM14 | — | — | 127 | 1.5 | 5% (S) | 10 (Tris) | 7.4 | 404 |
| VYFORM15 | — | — | 133 | 1.5 | 7% (S) | 10 (Tris) | 7.4 | 432 |
| VYFORM16 | — | — | 118 | 1.5 | 7% (S) | 10 (Tris) | 7.4 | 413 |
| VYFORM17 | — | — | 127 | 1.5 | 9% (S) | 10 (Tris) | 7.4 | 436 |
| VYFORM18 | — | — | 109 | 1.5 | 9% (S) | 10 (Tris) | 7.4 | 410 |
| VYFORM19 | — | — | 100 | 1.5 | 7% (S) | 10 (Tris); | 8.0 | — |
| | | | | | | 6.3 (HCl) | | |
| VYFORM20 | — | — | 100 | 1.5 | 7% (S) | 10 (Tris); | 7.5 | — |
| | | | | | | 9 (HCl) | | |
| VYFORM21 | — | — | 75 | — | 5% (S) | 10 (Tris) | — | — |
| VYFORM22 | — | — | 150 | — | 5% (S) | 10 (Tris) | — | — |
| VYFORM23 | — | — | 150 | — | 5% (S) | 10 (Tris); | — | — |
| | | | | | | 10 (MgCl₂₎ | | |
| VYFORM24 | — | — | 75 | — | 5% (S) | 10 (Tris); | — | — |
| | | | | | | 75 (Arg) | | |
| VYFORM25 | — | — | 150 | — | 5% (So) | 10 (Tris) | — | — |
| VYFORM26 | — | — | 150 | — | 5% (S) | 10 (His) | — | — |
| VYFORM27 | — | 1.5 | — | — | 7% (S) | 10 (Tris) | 8.0 | — |
| VYFORM28 | — | — | — | 75 | 5% (S) | 10 (Tris) | — | — |
| VYFORM29 | 10 | — | 180 | — | — | — | | |

S = Sucrose (sugar)
T = Trehalose (sugar)
So = Sorbitol (sugar alcohol)
His = Histidine (other)
Tris = tris(hydroxymethyl)aminomethane (other)
Arg = Arginine (other)

In certain embodiments, the formulation may include sodium phosphate, potassium phosphate, sodium chloride, sucrose, and optionally a copolymer such as Poloxamer 188 (e.g., Pluronic F-68). In certain embodiments, the formulation may include 10 mM sodium phosphate, 1.5 mM potassium phosphate, 100 mM sodium chloride, 5% w/v Sucrose, and optionally Poloxamer 188 (buffer pH of 7.5). In certain embodiments, the formulation may include 10 mM sodium phosphate, 1.5 mM potassium phosphate, 220 mM sodium chloride, 5% w/v Sucrose, and optionally Poloxamer 188 (buffer pH of 7.5). In certain embodiments, the formulation may include 10 mM sodium phosphate, 1.5 mM potassium phosphate, 100 mM sodium chloride, 7% w/v Sucrose, and optionally Poloxamer 188 (buffer pH of 7.5).

In certain embodiments, the formulation may include sodium phosphate, potassium phosphate, sodium chloride, potassium chloride, sucrose or trehalose, and optionally a copolymer such as Poloxamer 188 (e.g., Pluronic F-68).

In certain embodiments, the formulation may include potassium phosphate, sodium chloride, potassium chloride, Histidine, a sugar, and optionally a copolymer such as Poloxamer 188 (e.g., Pluronic F-68).

In certain embodiments, the formulation may include sodium chloride, potassium chloride, sucrose, Tris, and optionally a copolymer such as Poloxamer 188 (e.g. Pluronic F-68).

In certain embodiments, the formulation may include sodium chloride, potassium chloride, sucrose, Tris, hydrochloric acid, and optionally a copolymer such as Poloxamer 188 (e.g., Pluronic F-68).

In certain embodiments, the formulation may include sodium chloride, sucrose, Tris, and optionally a copolymer such as Poloxamer 188 (e.g., Pluronic F-68).

In certain embodiments, the formulation may include sodium chloride, sucrose, Tris, magnesium chloride, and optionally a copolymer such as Poloxamer 188 (e.g., Pluronic F-68).

In certain embodiments, the formulation may include sodium chloride, sucrose, Tris, arginine and optionally a copolymer such as Poloxamer 188 (e.g., Pluronic F-68).

In certain embodiments, the formulation may include sodium chloride, sorbitol, Tris, and optionally a copolymer such as Poloxamer 188 (e.g., Pluronic F-68).

In certain embodiments, the formulation may include sodium chloride, sucrose. Histidine and optionally a copolymer such as Poloxamer 188 (e.g., Pluronic F-68).

In certain embodiments, the formulation may include sodium chloride, sucrose, and optionally a copolymer such as Poloxamer 188 (e.g., Pluronic F-68). In certain embodiments, the formulation may include 105 mM sodium chloride, 5% (w/v) sucrose, and optionally a copolymer such as Poloxamer 188. In certain embodiments, the formulation may include 95 mM sodium chloride, 5% (w/v) sucrose, and optionally a copolymer such as Poloxamer 188. In certain embodiments, the formulation may include 220 mM sodium chloride, 5% (w/v) sucrose, and optionally a copolymer such as Poloxamer 188.

In certain embodiments, the formulation may include potassium phosphate, sucrose, tris and optionally a copolymer such as Poloxamer 188 (e.g., Pluronic F-68).

In certain embodiments, the formulation may include potassium chloride, sucrose, tris and optionally a copolymer such as Poloxamer 188 (e.g., Pluronic F-68).

In certain embodiments, the formulation may include sodium chloride, Tris, and optionally a copolymer such as Poloxamer 188 (e.g., Pluronic F-68). In certain embodiments, the formulation may include 100 mM sodium chloride, 20 mM Tris, and optionally a copolymer such as Poloxamer 188 (mixture pH of 8.0). In certain embodiments, the formulation may include 220 mM sodium chloride, 20 mM Tris, and optionally a copolymer such as Poloxamer 188 (mixture pH of 7.0-8.0). In certain embodiments, the formulation may include 290 mM sodium chloride, 20 mM Tris, and optionally a copolymer such as Poloxamer 188 (mixture pH of 8.0). In certain embodiments, the formulation may include 305 mM sodium chloride. 20 mM Tris, and optionally a copolymer such as Poloxamer 188 (mixture pH of 8.0). In certain embodiments, the formulation may include 2 M sodium chloride, 20 mM Tris, and optionally a copolymer such as Poloxamer 188 (mixture pH of 8.0). In certain embodiments, the formulation may include 170 mM sodium chloride, 40 mM Tris, and optionally a copolymer such as Poloxamer 188 (mixture pH of 8.5). In certain embodiments, the formulation may include 2 M sodium chloride, 1 M Tris, and optionally a copolymer such as Poloxamer 188 (mixture pH of 7.5).

In certain embodiments, the formulation may include sodium chloride, Tris-Bis Propane, and optionally a copolymer such as Poloxamer 188 (e.g., Pluronic F-68). In certain embodiments, the formulation may include 200 mM sodium chloride, 50 mM Tris-Bis Propane, and optionally a copolymer such as Poloxamer 188 (mixture pH of 9.0).

In certain embodiments, the formulation may include sodium phosphate, sodium chloride and optionally a copolymer such as Poloxamer 188. In certain embodiments, the formulation may include 10 mM sodium phosphate, 180 mM sodium chloride and optionally a copolymer such as Poloxamer 188 (mixture pH of 7.3). In certain embodiments, the formulation may include 20 mM sodium phosphate, 350 mM sodium chloride and optionally a copolymer such as Poloxamer 188 (mixture pH of 7.4). In certain embodiments, the formulation may include 50 mM sodium phosphate, 350 mM sodium chloride and optionally a copolymer such as Poloxamer 188 (mixture pH of 7.4).

In certain embodiments, the formulation may include sodium phosphate, potassium phosphate, potassium chloride, sodium chloride, and optionally a copolymer such as Poloxamer 188. In certain embodiments, the formulation may include 10 mM sodium phosphate, 2 mM Potassium Phosphate, 2.7 mM Potassium Chloride, 192 mM Sodium Chloride, and optionally a copolymer such as Poloxamer 188 (mixture pH of 7.5).

In certain embodiments, the formulation may include sodium citrate, sodium chloride and optionally a copolymer such as Poloxamer 188. In certain embodiments, the formulation may include 20 mM sodium citrate, 1 M sodium chloride and optionally a copolymer such as Poloxamer 188 (mixture pH of 6.0). In certain embodiments, the formulation may include 10 mM sodium citrate, 350 mM sodium chloride and optionally a copolymer such as Poloxamer 188 (mixture pH of 6.0). In certain embodiments, the formulation may include 20 mM sodium citrate. 350 mM sodium chloride and optionally a copolymer such as Poloxamer 188 (mixture pH of 3.0).

In certain embodiments, the formulation may include PBS. In certain embodiments, the formulation may include PBS and a sugar and/or a sugar substitute. The formulation may include 3-5% (w/v) of the sugar and/or sugar substitute to increase stability of the formulation. As a non-limiting example, the formulation is PBS and 3% (w/v) sucrose (VYFORM30). As another non-limiting example, the formulation is PBS and 5% (w/v) sucrose (VYFORM31). As another non-limiting example, the formulation is PBS and 7% (w/v) sucrose. In certain embodiments, the AAV particles of the disclosure may be formulated in PBS, in combination with an ethylene oxide/propylene oxide copolymer (also known as pluronic or poloxamer).

In certain embodiments, the AAV particles of the disclosure may be formulated in PBS with 3% (w/v) sucrose and 0.001%-0.1% (w/v) of Poloxamer 188 (e.g., Pluronic F-68).

In certain embodiments, the AAV particles of the disclosure may be formulated in PBS with 5% (w/v) sucrose and 0.001%-0.1% (w/v) of Poloxamer 188 (e.g., Pluronic F-68).

In certain embodiments, the AAV particles of the disclosure may be formulated in PBS with 0.001%-0.1% (w/v) of Poloxamer 188 (e.g., Pluronic F-68) at a pH of about 7.0.

In certain embodiments, the AAV particles of the disclosure may be formulated in PBS with 0.001%-0.1% (w/v) of Poloxamer 188 (e.g., Pluronic F-68) at a pH of about 7.3.

In certain embodiments, the AAV particles of the disclosure may be formulated in PBS with 0.001%-0.1%((w/v) of Poloxamer 188 (e.g., Pluronic F-68) at a pH of about 7.4.

In certain embodiments, the AAV particles of the disclosure may be formulated in a solution comprising sodium chloride, sodium phosphate and an ethylene oxide/propylene oxide copolymer.

In certain embodiments, the AAV particles of the disclosure may be formulated in a solution comprising 95 mM sodium chloride, 5 mM sodium phosphate dibasic. 5 mM sodium phosphate monobasic, 1.5 mM potassium phosphate, 7% w/v sucrose, and 0.001% poloxamer 188 (e.g., Pluronic F-68).

In certain embodiments, the AAV particles of the disclosure may be formulated in a solution comprising about 180 mM sodium chloride, about 10 mM sodium phosphate and about 0.001% poloxamer 188, at a pH of about 7.3. The concentration of sodium chloride in the final solution may be 150 mM-200 mM. As non-limiting examples, the concentration of sodium chloride in the final solution may be 150 mM, 160 mM, 170 mM, 180 mM, 190 mM or 200 mM. The concentration of sodium phosphate in the final solution may be 1 mM-50 mM. As non-limiting examples, the concentration of sodium phosphate in the final solution may be 1 mM, 2 mM, 3 mM, 4 mM, 5 mM, 6 mM, 7 mM, 8 mM, 9 mM, 10 mM, 15 mM, 20 mM, 25 mM, 30 mM, 40 mM, or 50 mM. The concentration of poloxamer 188 (Pluronic F-68) may be 0.0001%-1% (w/v). As non-limiting examples, the concentration of poloxamer 188 (Pluronic F-68) may be 0.0001%, 0.0005%, 0.001%, 0.005%, 0.01%, 0.05%, 0.1%, 0.5%, or 1% (w/V). The final solution may have a pH of 6.8-7.7. Non-limiting examples for the pH of the final solution include a pH of 6.8, 6.9, 7.0, 7.1, 7.2, 7.3, 7.4, 7.5, 7.6, or 7.7.

In certain embodiments, the AAV particles of the disclosure may be formulated in a solution comprising about 1.05% (w/v) sodium chloride, about 0.212% (w/v) sodium phosphate dibasic, heptahydrate, about 0.025% (w/v) sodium phosphate monobasic, monohydrate, and 0.001% (w/v) poloxamer 188, at a pH of about 7.4. As a non-limiting example, the concentration of AAV particle in this formulated solution may be about 0.001% (w/v). The concentration of sodium chloride in the final solution may be 0.1-2.0% (w/v), with non-limiting examples of 0.1%, 0.25%, 0.5%, 0.75%, 0.95%, 0.96%, 0.97%, 0.98%, 0.99%, 1.00%, 1.01%, 1.02%, 1.03%, 1.04%, 1.05%, 1.06%, 1.07%, 1.08%, 1.09%, 1.10%, 1.25%, 1.5%, 1.75%, or 2% (w/v). The concentration of sodium phosphate dibasic in the final solution may be 0.100-0.300% (w/v) with non-limiting examples including 0.100%, 0.125%, 0.150%, 0.175%, 0.200%, 0.210%, 0.211%, 0.212%, 0.213%, 0.214%, 0.215%, 0.225%, 0.250%, 0.275%, 0.300% (w/v). The concentration of sodium phosphate monobasic in the final solution may be 0.010-0.050% (w/v), with non-limiting examples of 0.010%, 0.015%, 0.020%, 0.021%, 0.022%, 0.023%, 0.024%, 0.025%, 0.026%, 0.027%, 0.028%, 0.029%, 0.030%, 0.035%, 0.040%, 0.045%, or 0.050% (w/v). The concentration of poloxamer 188 (Pluronic F-68) may be 0.0001%-1% (w/v). As non-limiting examples, the concentration of poloxamer 188 (Pluronic F-68)) may be 0.0001%, 0.0005%, 0.001%, 0.005%, 0.01%, 0.05%, 0.1%, 0.5%, or 1% (w/v). The final solution may have a pH of 6.8-7.7. Non-limiting examples for the pH of the final solution include a pH of 6.8, 6.9, 7.0, 7.1, 7.2, 7.3, 7.4, 7.5, 7.6, or 7.7.

In certain embodiments, the formulation comprises components with the following CAS (Chemical Abstracts Services) Registry Numbers, 7647-14-15 (sodium chloride), 7782-85-6 (sodium phosphate dibasic, heptahydrate), 10049-21-5 (sodium phosphate monobasic, monohydrate), and 9003-11-6 (poloxamer 188).

### Injectable Formulations

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of active ingredients, it is often desirable to slow the absorption of active ingredients from subcutaneous or intramuscular injections. This may be accomplished by the use of liquid suspensions of crystalline or amorphous material with poor water solubility. The rate of absorption of active ingredients depends upon the rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

### Depot Formulations

In certain embodiments of the present disclosure, AAV particle formulations of the present disclosure are formulated in depots for extended release. Generally, specific organs or tissues (“target tissues”) are targeted for administration.

In certain embodiments of the disclosure, pharmaceutical compositions, AAV particle formulations of the present disclosure are spatially retained within or proximal to target tissues. Provided are methods of providing pharmaceutical compositions, AAV particle formulations, to target tissues of mammalian subjects by contacting target tissues (which comprise one or more target cells) with pharmaceutical compositions, AAV particle formulations, under conditions such that they are substantially retained in target tissues, meaning that at least 10, 20, 30, 40, 50, 60, 70, 80, 85, 90, 95, 96, 97, 98, 99, 99.9, 99.99 or greater than 99.99% of the composition is retained in the target tissues. Advantageously, retention is determined by measuring the amount of pharmaceutical compositions, AAV particle formulations, that enter one or more target cells. For example, at least 1%, 5%, 10%, 20%, 30%, 40%, 50%, 60%, 70%, 80%, 85%, 90%, 95%, 96%, 97%, 98%, 99%, 99.9%. 99.99% or greater than 99.99% of pharmaceutical compositions, AAV particle formulations, administered to subjects are present intracellularly at a period of time following administration.

Certain aspects of the disclosure are directed to methods of providing pharmaceutical compositions, AAV particle formulations of the present disclosure to a target tissues of mammalian subjects, by contacting target tissues (comprising one or more target cells) with pharmaceutical compositions, AAV particle formulations under conditions such that they are substantially retained in such target tissues. Pharmaceutical compositions. AAV particles comprise enough active ingredient such that the effect of interest is produced in at least one target cell.

### Measurement and Analysis

Expression of payloads or the downregulating effect of such payloads from viral genomes may be determined using various methods known in the art such as, but not limited to immunochemistry (e.g., IHC), in situ hybridization (ISH), enzyme-linked immunosorbent assay (ELISA), affinity ELISA, ELISPOT, flow cytometry, immunocytology, surface plasmon resonance analysis, kinetic exclusion assay, liquid chromatography-mass spectrometry (LCMS), high-performance liquid chromatography (HPLC). BCA assay, immunoelectrophoresis, Western blot. SDS-PAGE, protein immunoprecipitation, and/or PCR.

### IV. Administration

The AAV particles comprising a nucleic acid sequence encoding the siRNA molecules of the present disclosure may be administered by any route which results in a therapeutically effective outcome. These include, but are not limited to, within the parenchyma of an organ such as, but not limited to, a brain (e.g., intraparenchymal), corpus striatum (intrastriatal), enteral (into the intestine), gastroenteral, epidural, oral (by way of the mouth), transdermal, peridural, intracerebral (into the cerebrum), intracerebroventricular (into the cerebral ventricles), subpial (under the pia), epicutaneous (application onto the skin), intradermal. (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraganglionic (into the ganglion), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal.

In specific embodiments, compositions of AAV particles comprising a nucleic acid sequence encoding the siRNA molecules of the present disclosure may be administered in a way which facilitates the vectors or siRNA molecule to enter the central nervous system and penetrate into medium spiny and/or cortical neurons and/or astrocytes.

In some embodiments, the AAV particles comprising a nucleic acid sequence encoding the siRNA molecules of the present disclosure may be administered by intramuscular injection.

In some embodiments, the AAV particles comprising a nucleic acid sequence encoding the siRNA molecules of the present disclosure may be administered via intraparenchymal injection.

In some embodiments, the AAV particles comprising a nucleic acid sequence encoding the siRNA molecules of the present disclosure may be administered via intraparenchymal injection and intrathecal injection.

In some embodiments, the AAV particles comprising a nucleic acid sequence encoding the siRNA molecules of the present disclosure may be administered via intrastriatal injection.

In some embodiments, the AAV particles comprising a nucleic acid sequence encoding the siRNA molecules of the present disclosure may be administered via intrastriatal injection and another route of administration described herein.

In some embodiments, AAV particles that express siRNA duplexes of the present disclosure may be administered to a subject by peripheral injections (e.g., intravenous) and/or intranasal delivery. It was disclosed in the art that the peripheral administration of AAV particles for siRNA duplexes can be transported to the central nervous system, for example, to the neurons (e.g., U.S. Patent Publication Nos. 20100240739; and 20100130594; the content of each of which is incorporated herein by reference in their entirety).

In other embodiments, compositions comprising at least one AAV particle comprising a nucleic acid sequence encoding the siRNA molecules of the present disclosure may be administered to a subject by intracranial delivery (See, e.g., U.S. Pat. No. 8,119,611; the content of which is incorporated herein by reference in its entirety).

The AAV particle comprising a nucleic acid sequence encoding the siRNA molecules of the present disclosure may be administered in any suitable form, either as a liquid solution or suspension, as a solid form suitable for liquid solution or suspension in a liquid solution. The siRNA duplexes may be formulated with any appropriate and pharmaceutically acceptable excipient.

The AAV particle comprising a nucleic acid sequence encoding the siRNA molecules of the present disclosure may be administered in a “therapeutically effective” amount, i.e., an amount that is sufficient to alleviate and/or prevent at least one symptom associated with the disease, or provide improvement in the condition of the subject.

In some embodiments, the AAV particle may be administered to the cisterna magna in a therapeutically effective amount to transduce medium spiny neurons, cortical neurons and/or astrocytes. As a non-limiting example, the vector may be administered intrathecally.

In some embodiments, the AAV particle may be administered using intrathecal infusion in a therapeutically effective amount to transduce medium spiny neurons, cortical neurons and/or astrocytes. As a non-limiting example, the vector may be administered intrathecally.

In some embodiments, the AAV particle comprising a modulatory polynucleotide may be formulated. As a non-limiting example, the baricity and/or osmolality of the formulation may be optimized to ensure optimal drug distribution in the central nervous system or a region or component of the central nervous system.

In some embodiments, the AAV particle comprising a modulatory polynucleotide may be delivered to a subject via a single route of administration.

In some embodiments, the AAV particle comprising a modulatory polynucleotide may be delivered to a subject via a multi-site route of administration. A subject may be administered the AAV particle comprising a modulatory polynucleotide at 2, 3, 4, 5 or more than 5 sites.

In some embodiments, a subject may be administered the AAV particle comprising a modulatory polynucleotide described herein using a bolus injection.

In some embodiments, a subject may be administered the AAV particle comprising a modulatory polynucleotide described herein using sustained delivery over a period of minutes, hours, or days. The infusion rate may be changed depending on the subject, distribution, formulation, or another delivery parameter.

In some embodiments, the AAV particle described herein is administered via putamen and caudate infusion. As a non-limiting example, the dual infusion provides a broad striatal distribution as well as a frontal and temporal cortical distribution.

In some embodiments, the AAV particle is AAV-DJ8 which is administered via unilateral putamen infusion. As a non-limiting example, the distribution of the administered AAV-DJ8 is similar to the distribution of AAV1 delivered via unilateral putamen infusion.

In some embodiments, the AAV particle described herein is administered via intrathecal (IT) infusion at C1. The infusion may be for 1, 2, 3, 4, 6, 7, 8, 9, 10, 11, 12, 13, 14, 15 or more than 15 hours.

In some embodiments, the selection of subjects for administration of the AAV particle described herein and/or the effectiveness of the dose, route of administration and/or volume of administration may be evaluated using imaging of the perivascular spaces (PVS) which are also known as Virchow-Robin spaces. PVS surround the arterioles and venules as they perforate brain parenchyma and are filled with cerebrospinal fluid (CSF)/interstitial fluid. PVS are common in the midbrain, basal ganglia, and centrum semiovale. While not wishing to be bound by theory, PVS may play a role in the normal clearance of metabolites and have been associated with worse cognition and several disease states including Parkinson's disease. PVS are usually are normal in size but they can increase in size in a number of disease states. Potter et al. (Cerebrovasc Dis. 2015 January; 39(4): 224-231; the contents of which are herein incorporated by reference in its entirety) developed a grading method where they studied a full range of PVS and rated basal ganglia, centrum semiovale and midbrain PVS. They used the frequency and range of PVS used by Mac and Lullich et al. (J Neurol Neurosurg Psychiatry. 2004 November; 75(11):1519-23; the contents of which are herein incorporated by reference in its entirety) and Potter et al. gave 5 ratings to basal ganglia and centrum semiovale PVS: 0 (none), 1 (1-10), 2 (11-20), 3 (21-40) and 4 (>40) and 2 ratings to midbrain PVS: 0 (non-visible) or 1 (visible). The user guide for the rating system by Potter et al. can be found at: www.sbirc.ed.ac.uk/documents/epvs-rating-scale-user-guide.pdf.

In some embodiments, AAV particles described herein is administered via thalamus infusion. Infusion into the thalamus may be bilateral or unilateral.

In some embodiments, AAV particles described herein are administered via putamen infusion. Infusion into the thalamus may be bilateral or unilateral.

In some embodiments, AAV particles described herein are administered via putamen and thalamus infusion. Dual infusion into the putamen and thalamus may maximize brain distribution via axonal transport to cortical areas. Evers et al. observed positive transduction of neurons in the motor cortex and part of the parietal cortex after bilateral injections of AAV5-GFP into the putamen and thalamus of tgHD minipigs (Molecular Therapy (2018), doi: 10.1016/j.ymthe.2018.06.021). Infusion into the putamen and thalamus may be independently bilateral or unilateral. As a non-limiting example, AAV particles may be infused into the putamen and thalamus from both sides of the brain. As another non-limiting example, AAV particles may be infused into the left putamen and left thalamus, or right putamen and right thalamus. As yet another non-limiting example, AAV particles may be infused into the left putamen and right thalamus, or right putamen and left thalamus. Dual infusion may occur consecutively or simultaneously.

In some embodiments, the AAV particle comprising a modulatory polynucleotide may be delivered to a subject in the absence of gene therapy-related changes in body weight.

In some embodiments, the AAV particle comprising a modulatory polynucleotide may be delivered to a subject in the absence of gene therapy-related clinical signs, including but not limited to incoordination, inappetence, decreased feeding, and overall weakness.

In some embodiments, the AAV particle comprising a modulatory polynucleotide may be delivered to a subject in the absence of gene therapy-related changes to blood of a subject. In certain embodiments, the changes in blood of a subject are serum chemistry, and coagulation parameters.

In some embodiments, the AAV particle comprising a modulatory polynucleotide may be delivered to a subject in the absence of pathological changes to a tissue of a subject (e.g., brain of the subject). In certain embodiments the pathological change is a gross pathological change, such as, but not limited to, atrophy. In certain embodiments, the pathological change is a histopathological change, including but not limited to, target specific (e.g., HTT) inclusions.

### V. Methods of Use

### General

The present disclosure provides a method for treating a disease, disorder and/or condition in a mammalian subject, including a human subject, comprising administering to the subject any of the viral particles or formulations described herein or administering to the subject any of the described compositions, including pharmaceutical compositions or formulations, described herein.

In certain embodiments, administration of the formulated AAV particles to a subject with not change the course of the underlying disease but will ameliorate symptoms in a subject.

In certain embodiments, the viral particles of the present disclosure are administered to a subject prophylactically.

In certain embodiments, the viral particles of the present disclosure are administered to a subject having at least one of the diseases described herein.

In certain embodiments, the viral particles of the present disclosure are administered to a subject to treat a disease or disorder described herein. The subject may have the disease or disorder or may be at-risk to developing the disease or disorder.

The present disclosure provides a method for administering to a subject in need thereof, including a human subject, a therapeutically effective amount of the AAV particles of the present disclosure to slow, stop or reverse disease progression. As a non-limiting example, disease progression may be measured by tests or diagnostic tool(s) known to those skilled in the art. As another non-limiting example, disease progression may be measured by change in the pathological features of the brain. CSF, or other tissues of the subject.

In certain embodiments, various non-infectious diseases, including neurological diseases, may be treated with pharmaceutical compositions of the present disclosure. AAV particles, especially blood brain barrier crossing AAV particles of the present disclosure, are particularly useful in treating various neurological diseases. As a non-limiting example, the neurological disease may be Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS—Neurological Complications, Alexander Disease, Alpers' Disease, Alternating Hemiplegia, Alzheimer's Disease, Amyotrophic Lateral Sclerosis (ALS), Anencephaly, Aneurysm, Angelman Syndrome, Angiomatosis, Anoxia. Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becker's Myotonia, Behcet's Disease. Bell's Palsy, Benign Essential Blepharospasm, Benign Focal Amyotrophy, Benign Intracranial Hypertension, Bernhardt-Roth Syndrome, Binswanger's Disease, Blepharospasm. Bloch-Sulzberger Syndrome, Brachial Plexus Birth Injuries. Brachial Plexus Injuries, Bradbury-Eggleston Syndrome, Brain and Spinal Tumors, Brain Aneurysm, Brain Injury. Brown-Sequard Syndrome, Bulbospinal Muscular Atrophy, Cerebral Autosomal Dominant Arteriopathy with Sub-cortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavernous Malformation. Cerebral Gigantism, Cerebral Hypoxia, Cerebral Palsy, Cerebro-Oculo-Facio-Skeletal Syndrome (COFS), Charcot-Marie-Tooth Disease, Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome Type II, Coffin Lowry Syndrome, Colpocephaly, Coma, Complex Regional Pain Syndrome, Congenital Facial Diplegia, Congenital Myasthenia, Congenital Myopathy, Congenital Vascular Cavernous Malformations. Corticobasal Degeneration. Cranial Arteritis, Craniosynostosis, Cree encephalitis, Creutzfeldt-Jakob Disease, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, De Morsier's Syndrome, Dejerine-Klumpke Palsy, Dementia, Dementia-Multi-Infarct, Dementia—Semantic, Dementia-Subcortical, Dementia With Lewy Bodies, Dentate Cerebellar Ataxia, Dentatorubral Atrophy, Dermatomyositis, Developmental Dyspraxia. Devic's Syndrome, Diabetic Neuropathy, Diffuse Sclerosis, Dravet Syndrome, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalopathy, Encephalopathy (familial infantile), Encephalotrigeminal Angiomatosis, Epilepsy, Epileptic Hemiplegia, Erb's Palsy, Erb-Duchenne and Dejerine-Klumpke Palsies, Essential Tremor, Extrapontine Myelinolysis, Fabry Disease, Fahr's Syndrome. Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Friedreich's Ataxia, Frontotemporal Dementia, Gaucher Disease, Generalized Gangliosidoses, Gerstmann's Syndrome, Gerstmann-Straussler-Scheinker Disease, Giant Axonal Neuropathy, Giant Cell Arteritis, Giant Cell Inclusion Disease, Globoid Cell Leukodystrophy, Glossopharyngeal Neuralgia, Glycogen Storage Disease, Guillain-Barré Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hydranencephaly, Hydrocephalus, Hydrocephalus—Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension. Isaacs' Syndrome, Joubert Syndrome, Kearns-Sayre Syndrome, Kennedy's Disease. Kinsbourne syndrome, Kleine-Levin Syndrome, Klippel-Feil Syndrome, Klippel-Trenaunay Syndrome (KTS), Klüver-Bucy Syndrome, Korsakoff's Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities. Leigh's Disease, Lennox-Gastaut Syndrome, Lesch-Nyhan Syndrome, Leukodystrophy, Levine-Critchley Syndrome, Lewy Body Dementia, Lipid Storage Diseases, Lipoid Proteinosis, Lissencephaly, Locked-In Syndrome, Lou Gehrig's Disease, Lupus—Neurological Sequelae, Lyme Disease-Neurological Complications, Machado-Joseph Disease, Macrencephaly, Megalencephaly, Melkersson-Rosenthal Syndrome, Meningitis, Meningitis and Encephalitis, Menkes Disease, Meralgia Paresthetica, Metachromatic Leukodystrophy, Microcephaly, Migraine, Miller Fisher Syndrome, Mini Stroke, Mitochondrial Myopathy, Moebius Syndrome, Monomelic Amyotrophy, Motor Neuron Diseases, Moyamoya Disease, Mucolipidoses, Mucopolysaccharidoses, Multi-Infarct Dementia. Multifocal Motor Neuropathy, Multiple Sclerosis, Multiple System Atrophy, Multiple System Atrophy with Orthostatic Hypotension, Muscular Dystrophy, Myasthenia—Congenital, Myasthenia Gravis, Myelinoclastic Diffuse Sclerosis, Myoclonic Encephalopathy of Infants, Myoclonus, Myopathy, Myopathy-Congenital, Myopathy-Thyrotoxic, Myotonia, Myotonia Congenita, Narcolepsy, Neuroacanthocytosis, Neurodegeneration with Brain Iron Accumulation, Neurofibromatosis. Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathy—Hereditary, Neurosarcoidosis, Neurosyphilis, Neurotoxicity, Nevus Cavernosus, Niemann-Pick Disease, O'Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain—Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Periodic Paralyses, Peripheral Neuropathy, Periventricular Leukomalacia. Persistent Vegetative State, Pervasive Developmental Disorders, Phytanic Acid Storage Disease, Pick's Disease, Pinched Nerve. Piriformis Syndrome, Pituitary Tumors, Polymyositis. Pompe Disease, Porencephaly, Post-Polio Syndrome, Postherpetic Neuralgia, Postinfectious Encephalomyelitis, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases, Progressive Hemifacial Atrophy, Progressive Locomotor Ataxia, Progressive Multifocal Leukoencephalopathy, Progressive Sclerosing Poliodystrophy, Progressive Supranuclear Palsy, Prosopagnosia, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease—Infantile, Repetitive Motion Disorders, Repetitive Stress Injuries, Restless Legs Syndrome, Retrovirus-Associated Myelopathy, Rett Syndrome, Reye's Syndrome. Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjögren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Atrophy, Spinocerebellar Degeneration, Steele-Richardson-Olszewski Syndrome, Stiff-Person Syndrome, Striatonigral Degeneration, Stroke. Sturge-Weber Syndrome, Subacute Sclerosing Panencephalitis, Subcortical Arteriosclerotic Encephalopathy, Short-lasting, Unilateral, Neuralgiform (SUNCT) Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis. Vascular Erectile Tumor, Vasculitis Syndromes of the Central and Peripheral Nervous Systems, Von Economo's Disease, Von Hippel-Lindau Disease (VHL), Von Recklinghausen's Disease, Wallenberg's Syndrome, Werdnig-Hoffman Disease, Wernicke-Korsakoff Syndrome, West Syndrome. Whiplash, Whipple's Disease, Williams Syndrome, Wilson Disease, Wolman's Disease, X-Linked Spinal and Bulbar Muscular Atrophy.

The present disclosure additionally provides a method for treating neurological disorders in a mammalian subject, including a human subject, comprising administering to the subject any of the AAV particles or pharmaceutical compositions of the present disclosure. In certain embodiments, the AAV particle is a blood brain barrier crossing particle. In certain embodiments, neurological disorders treated according to the methods described herein include, but are not limited to Amyotrophic lateral sclerosis (ALS). Huntington's Disease (HD), Parkinson's Disease (PD), and/or Friedreich's Ataxia (FA).

### Kits and Devices

### Kits

In some embodiments, the disclosure provides a variety of kits for conveniently and/or effectively carrying out methods of the present disclosure. Typically, kits will comprise sufficient amounts and/or numbers of components to allow a user to perform multiple treatments of a subject(s) and/or to perform multiple experiments.

Any of the AAV particles of the present disclosure may be comprised in a kit. In some embodiments, kits may further include reagents and/or instructions for creating and/or synthesizing compounds and/or compositions of the present disclosure. In some embodiments, kits may also include one or more buffers. In some embodiments, kits of the disclosure may include components for making protein or nucleic acid arrays or libraries and thus, may include, for example, solid supports.

In some embodiments, kit components may be packaged either in aqueous media or in lyophilized form. The container means of the kits will generally include at least one vial, test tube, flask, bottle, syringe, or other container means, into which a component may be placed, and preferably, suitably aliquoted. Where there is more than one kit component, (labeling reagent and label may be packaged together), kits may also generally contain second, third or other additional containers into which additional components may be separately placed. In some embodiments, kits may also comprise second container means for containing sterile, pharmaceutically acceptable buffers and/or other diluents. In some embodiments, various combinations of components may be comprised in one or more vial. Kits of the present disclosure may also typically include means for containing compounds and/or compositions of the present disclosure, e.g., proteins, nucleic acids, and any other reagent containers in close confinement for commercial sale. Such containers may include injection or blow-molded plastic containers into which desired vials are retained.

In some embodiments, kit components are provided in one and/or more liquid solutions. In some embodiments, liquid solutions are aqueous solutions, with sterile aqueous solutions being particularly preferred. In some embodiments, kit components may be provided as dried powder(s). When reagents and/or components are provided as dry powders, such powders may be reconstituted by the addition of suitable volumes of solvent. In some embodiments, it is envisioned that solvents may also be provided in another container means. In some embodiments, labeling dyes are provided as dried powders. In some embodiments, it is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 micrograms or at least or at most those amounts of dried dye are provided in kits of the disclosure. In such embodiments, dye may then be resuspended in any suitable solvent, such as DMSO.

In some embodiments, kits may include instructions for employing kit components as well the use of any other reagent not included in the kit. Instructions may include variations that may be implemented.

### Devices

In some embodiments, the AAV particles may delivered to a subject using a device to deliver the AAV particles and a head fixation assembly. The head fixation assembly may be, but is not limited to, any of the head fixation assemblies sold by MRI interventions. As a non-limiting example, the head fixation assembly may be any of the assemblies described in U.S. Pat. Nos. 8,099,150, 8,548,569 and 9,031,636 and International Patent Publication Nos. WO201108495 and WO2014014585, the contents of each of which are incorporated by reference in their entireties. A head fixation assembly may be used in combination with an MRI compatible drill such as, but not limited to, the MRI compatible drills described in International Patent Publication No. WO2013181008 and US Patent Publication No. US20130325012, the contents of which are herein incorporated by reference in its entirety.

In some embodiments, the AAV particles may be delivered using a method, system and/or computer program for positioning apparatus to a target point on a subject to deliver the AAV particles. As a non-limiting example, the method, system and/or computer program may be the methods, systems and/or computer programs described in U.S. Pat. No. 8,340,743, the contents of which are herein incorporated by reference in its entirety. The method may include: determining a target point in the body and a reference point, wherein the target point and the reference point define a planned trajectory line (PTL) extending through each; determining a visualization plane, wherein the PTL intersects the visualization plane at a sighting point; mounting the guide device relative to the body to move with respect to the PTL, wherein the guide device does not intersect the visualization plane; determining a point of intersection (GPP) between the guide axis and the visualization plane; and aligning the GPP with the sighting point in the visualization plane.

In some embodiments, the AAV particles may be delivered to a subject using a convention-enhanced delivery device. Non-limiting examples of targeted delivery of drugs using convection are described in US Patent Publication Nos. US20100217228, US20130035574 and US20130035660 and International Patent Publication No. WO2013019830 and WO2008144585, the contents of each of which are herein incorporated by reference in their entireties.

In some embodiments, a subject may be imaged prior to, during and/or after delivery of the AAV particles. The imaging method may be a method known in the art and/or described herein, such as but not limited to, magnetic resonance imaging (MRI). As a non-limiting example, imaging may be used to assess therapeutic effect. As another non-limiting example, imaging may be used for assisted delivery of AAV particles.

In some embodiments, the AAV particles may be delivered using an MRI-guided device. Non-limiting examples of MRI-guided devices are described in U.S. Pat. Nos. 9,055,884, 9,042,958, 8,886,288, 8,768.433, 8,396,532, 8,369,930, 8,374,677 and 8,175,677 and US Patent Application No. US20140024927 the contents of each of which are herein incorporated by reference in their entireties. As a non-limiting example, the MRI-guided device may be able to provide data in real time such as those described in U.S. Pat. Nos. 8,886,288 and 8,768,433, the contents of each of which is herein incorporated by reference in its entirety. As another non-limiting example, the MRI-guided device or system may be used with a targeting cannula such as the systems described in U.S. Pat. Nos. 8,175,677 and 8,374,677, the contents of each of which are herein incorporated by reference in their entireties. As yet another non-limiting example, the MRI-guided device includes a trajectory guide frame for guiding an interventional device as described, for example, in U.S. Pat. No. 9,055,884 and US Patent Application No. US20140024927, the contents of each of which are herein incorporated by reference in their entireties.

In some embodiments, the AAV particles may be delivered using an MRI-compatible tip assembly. Non-limiting examples of MRI-compatible tip assemblies are described in US Patent Publication No. US20140275980, the contents of which is herein incorporated by reference in its entirety.

In some embodiments, the AAV particles may be delivered using a cannula which is MRI-compatible. Non-limiting examples of MRI-compatible cannulas include those taught in International Patent Publication No. WO2011130107, the contents of which are herein incorporated by reference in its entirety. In some embodiments, the cannula or a portion thereof or the tubing associated with the cannula is attached, mounted, glued, affixed or otherwise makes reversible contact with the tissue surrounding the surgical site/field. Such contact may be localized and/or stabilized in one position during all or a portion of the procedure.

In some embodiments, the AAV particles may be delivered using a catheter which is MRI-compatible. Non-limiting examples of MRI-compatible catheters include those taught in International Patent Publication No. WO2012116265, U.S. Pat. No. 8,825,133 and US Patent Publication No. US20140024909, the contents of each of which are herein incorporated by reference in their entireties.

In some embodiments, the AAV particles may be delivered using a device with an elongated tubular body and a diaphragm as described in US Patent Publication Nos. US20140276582 and US20140276614, the contents of each of which are herein incorporated by reference in their entireties.

In some embodiments, the AAV particles may be delivered using an MRI compatible localization and/or guidance system such as, but not limited to, those described in US Patent Publication Nos. US20150223905 and US20150230871, the contents of each of which are herein incorporated by reference in their entireties. As a non-limiting example, the MRI compatible localization and/or guidance systems may comprise a mount adapted for fixation to a patient, a targeting cannula with a lumen configured to attach to the mount so as to be able to controllably translate in at least three dimensions, and an elongate probe configured to snugly advance via slide and retract in the targeting cannula lumen, the elongate probe comprising at least one of a stimulation or recording electrode.

In some embodiments, the AAV particles may be delivered to a subject using a trajectory frame as described in US Patent Publication Nos. US20150031982 and US20140066750 and International Patent Publication Nos. WO2015057807 and WO2014039481, the contents of each of which are herein incorporated by reference in their entireties.

In some embodiments, the AAV particles may be delivered to a subject using a gene gun.

### Use of AAV Particles Encoding Protein Payloads

Provided in the present disclosure are methods for introducing into cells the AAV particles manufactured according to the methods and systems of the present disclosure, the methods comprising introducing into said cells any of the vectors in an amount sufficient for an increase in the production of target mRNA and protein to occur. In some aspects, the cells may be muscle cells, stem cells, neurons such as but not limited to, motor, hippocampal, entorhinal, thalamic or cortical neurons, and glial cells such as astrocytes or microglia.

Disclosed in the present disclosure are methods for treating neurological disease associated with insufficient function/presence of a target protein in a subject in need of treatment. The method optionally includes administering to the subject a therapeutically effective amount of a composition comprising AAV particles of the present disclosure. As a non-limiting example, the AAV particles can increase target gene expression, increase target protein production, and thus reduce one or more symptoms of neurological disease in the subject such that the subject is therapeutically treated.

In certain embodiments, the AAV particle of the present disclosure comprising a nucleic acid encoding a protein payload includes an AAV capsid that allows for transmission across the blood brain barrier after intravenous administration.

In certain embodiments, the composition comprising the AAV particles of the present disclosure is administered to the central nervous system of the subject via systemic administration. In certain embodiments, the systemic administration is intravenous injection.

In certain embodiments, the composition comprising the AAV particles of the present disclosure is administered to the central nervous system of the subject. In certain embodiments, the composition comprising the AAV particles of the present disclosure is administered to a tissue of a subject (e.g., brain of the subject).

In certain embodiments, the composition comprising the AAV particles of the present disclosure is administered to the central nervous system of the subject via intraparenchymal injection. Non-limiting examples of intraparenchymal injections include intrathalamic, intrastriatal, intrahippocampal or targeting the entorhinal cortex.

In certain embodiments, the composition comprising the AAV particles of the present disclosure is administered to the central nervous system of the subject via intraparenchymal injection and intrathecal injection.

In certain embodiments, the AAV particles of the present disclosure may be delivered into specific types of targeted cells, including, but not limited to, hippocampal, cortical, motor, or entorhinal neurons; glial cells including oligodendrocytes, astrocytes and microglia; and/or other cells surrounding neurons such as T cells.

In certain embodiments, the AAV particles of the present disclosure may be delivered to neurons in the striatum (e.g., putamen) and/or cortex.

In certain embodiments, the AAV particles of the present disclosure may be used as a therapy for neurological disease.

In certain embodiments, the AAV particles of the present disclosure may be used to increase target protein and reduce symptoms of neurological disease in a subject. The increase of target protein and/or the reduction of symptoms of neurological disease may be, independently, altered (increased for the production of target protein and reduced for the symptoms of neurological disease) by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 540%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%. 10-55%, 10-60%, 10-65%, 10-70%, 10-75%, 10-80%, 10-85%, 10-90%, 10-95%, 15-25%, 15-30%, 15-35%, 1540%, 1545%, 15-50%, 15-55%, 15-60%, 15-65%, 15-70%, 15-75%, 15-80%, 15-85%, 15-90%, 15-95%, 20-30%, 20-35%, 20-40%, 2045%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 3040%, 3045%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

### Use of AAV Particles Comprising RNAi Polynucleotides

Provided in the present disclosure are methods for introducing the AAV particles, comprising a nucleic acid sequence encoding the siRNA molecules of the present disclosure into cells, the method comprising introducing into said cells any of the vectors in an amount sufficient for degradation of a target mRNA to occur, thereby activating target-specific RNAi in the cells. In some aspects, the cells may be muscle cells, stem cells, neurons such as but not limited to, motor, hippocampal, entorhinal, thalamic, or cortical neurons, and glial cells such as astrocytes or microglia.

Disclosed in the present disclosure are methods for treating neurological diseases associated with dysfunction of a target protein in a subject in need of treatment. The method optionally includes administering to the subject a therapeutically effective amount of a composition comprising AAV particles comprising a nucleic acid sequence encoding the siRNA molecules of the present disclosure. As a non-limiting example, the siRNA molecules can silence target gene expression, inhibit target protein production, and reduce one or more symptoms of neurological disease in the subject such that the subject is therapeutically treated.

In certain embodiments, the composition comprising the AAV particles of the present disclosure comprising a nucleic acid sequence encoding siRNA molecules include an AAV capsid that allows for transmission across the blood brain barrier after intravenous administration.

In certain embodiments, the composition comprising the AAV particles comprising a nucleic acid sequence encoding the siRNA molecules of the present disclosure is administered to the central nervous system of the subject. In certain embodiments, the composition comprising the AAV particles comprising a nucleic acid sequence encoding the siRNA molecules of the present disclosure is administered to a tissue of a subject (e.g., brain of the subject).

In certain embodiments, the composition comprising the AAV particles comprising a nucleic acid sequence encoding the siRNA molecules of the present disclosure is administered to the central nervous system of the subject via systemic administration. In certain embodiments, the systemic administration is intravenous injection.

In certain embodiments, the composition comprising the AAV particles comprising a nucleic acid sequence encoding the siRNA molecules of the present disclosure is administered to the central nervous system of the subject via intraparenchymal injection. Non-limiting examples of intraparenchymal injections include intrathalamic, intrastriatal, intrahippocampal or targeting the entorhinal cortex.

In certain embodiments, the composition comprising the AAV particles comprising a nucleic acid sequence encoding the siRNA molecules of the present disclosure is administered to the central nervous system of the subject via intraparenchymal injection and intrathecal injection.

In certain embodiments, the AAV particles comprising a nucleic acid sequence encoding the siRNA molecules of the present disclosure may be delivered into specific types of targeted cells, including, but not limited to, hippocampal, cortical, motor, or entorhinal neurons; glial cells including oligodendrocytes, astrocytes, and microglia; and/or other cells surrounding neurons such as T cells.

In certain embodiments, the AAV particles comprising a nucleic acid sequence encoding the siRNA molecules of the present disclosure may be delivered to neurons in the striatum and/or cortex.

In certain embodiments, the AAV particles comprising a nucleic acid sequence encoding the siRNA molecules of the present disclosure may be used as a therapy for neurological disease.

In certain embodiments, the AAV particles comprising a nucleic acid sequence encoding the siRNA molecules of the present disclosure may be used as a therapy for Amyotrophic Lateral Sclerosis.

In certain embodiments, the AAV particles comprising a nucleic acid sequence encoding the siRNA molecules of the present disclosure may be used as a therapy for Huntington's Disease.

In certain embodiments, the AAV particles comprising a nucleic acid sequence encoding the siRNA molecules of the present disclosure may be used as a therapy for Parkinson's Disease.

In certain embodiments, the AAV particles comprising a nucleic acid sequence encoding the siRNA molecules of the present disclosure may be used as a therapy for Friedreich's Ataxia.

In certain embodiments, the AAV particles comprising a nucleic acid sequence encoding the siRNA molecules of the present disclosure may be used to suppress a target in order to treat neurological disease. Target protein in astrocytes may be suppressed by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 2045%, 20-50%, 20-55%, 20-60%, 20-65%, 20-70%, 20-75%, 20-80%, 20-85%, 20-90%, 20-95%, 25-35%, 2540%, 2545%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 3545%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%. Target protein in astrocytes may be reduced may be 5%, 10%, 15%, 20%, 25%, 30%, 35%, 40%, 45%, 50%, 55%, 60%, 65%, 70%, 75%, 80%, 85%. 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 3045%, 30-50%, 30-55%, 30-60%, 30-65%, 30-70%, 30-75%, 30-80%, 30-85%, 30-90%, 30-95%, 3545%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In certain embodiments, administration of the AAV particles encoding a siRNA of the present disclosure, to a subject may lower target protein levels in a subject. The target protein levels may be lowered by about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS of a subject. As a non-limiting example, the AAV particles may lower the protein levels of a target protein by at least 50%. As a non-limiting example, the AAV particles may lower the proteins levels of a target protein by at least 40%.

### Therapeutic Indications

### Parkinson's Disease

Parkinson's Disease (PD) is a progressive disorder of the nervous system affecting especially the substantia nigra of the brain. PD develops are a result of the loss of dopamine producing brain cells. Typical early symptoms of PD include shaking or trembling of a limb, e.g., hands, arms, legs, feet, and face. Additional characteristic symptoms are stiffness of the limbs and torso, slow movement, or an inability to move, impaired balance and coordination, cognitional changes, and psychiatric conditions e.g., depression and visual hallucinations. PD has both familial and idiopathic forms and it is suggestion to be involved with genetic and environmental causes. PD affects more than 4 million people worldwide. In the US, approximately 60, 000 cases are identified annually. Generally, PD begins at the age of 50 or older. An early-onset form of the condition begins at age younger than 50, and juvenile-onset PD begins before age of 20.

Death of dopamine producing brain cells related to PD has been associated with aggregation, deposition, and dysfunction of alpha-synuclein protein (see, e.g., Marques and Outeiro, 2012. Cell Death Dis. 3:e350. Jenner. 1989, J Neurol Neurosurg Psychiatry. Special Supplement, 22-28, and references therein). Studies have suggested that alpha-synuclein has a role in presynaptic signaling, membrane trafficking and regulation of dopamine release and transport. Alpha-synuclein aggregates, e.g., in forms of oligomers, have been suggested to be species responsible for neuronal dysfunction and death. Mutations of the alpha-synuclein gene (SNCA) have been identified in the familial forms of PD, but also environmental factors, e.g., neurotoxin affect alpha-synuclein aggregation. Other suggested causes of brain cell death in PD are dysfunction of proteosomal and lysosomal systems, reduced mitochondrial activity.

PD is related to other diseases related to alpha-synuclein aggregation, referred to as “synucleinopathies.” Such diseases include, but are not limited to, Parkinson's Disease Dementia (PDD), multiple system atrophy (MSA), dementia with Lewy bodies, juvenile-onset generalized neuroaxonal dystrophy (Hallervorden-Spatz disease), pure autonomic failure (PAF), neurodegeneration with brain iron accumulation type-1 (NBIA-1) and combined Alzheimer's and Parkinson's disease.

As of today, no cure or prevention therapy for PD has been identified. A variety of drug therapies available provide relief to the symptoms. Non-limiting examples of symptomatic medical treatments include carbidopa and levodopa combination reducing stiffness and slow movement, and anticholinergics to reduce trembling and stiffness. Other optional therapies include, e.g., deep brain stimulation and surgery. There remains a need for therapy affecting the underlying pathophysiology. For example, antibodies targeting alpha-synuclein protein, or other proteins relevant for brain cell death in PD, may be used to prevent and/or treat PD.

In certain embodiments, methods of the present disclosure may be used to treat subjects suffering from PD and other synucleinopathies. In certain embodiments, methods of the present disclosure may be used to treat subjects suspected of developing PD and other synucleinopathies.

AAV particles, pharmaceutical formulations, and methods of using the viral particles described in the present disclosure may be used to prevent, manage and/or treat PD.

### Spinal Muscular Atrophy

Spinal muscular atrophy (SMA) is a hereditary disease-causing weakness and wasting of the voluntary muscles in the arms and legs of infants and children. SMA is associated with abnormalities in the protein production of the survival motor neuron gene 1 (SMN 1). Lack of the protein affects degeneration and death of lower motor neurons. Typical symptoms include floppy limbs and trunk, feeble movement of the arms and legs, difficulties in swallowing and eating, and impaired breathing. SMA is the most common genetic disorder leading to death of children under 2 years of age. SMA affects one in 6,000 to 10,000 people.

As of today, there is no cure for SMA. Therapies available are aimed at management of the symptoms and prevention of additional complications. Such therapies are associated e.g., with cardiology, movement management, respiratory care and mental health. There remains a need for therapy affecting the underlying pathophysiology of SMA and related diseases and ailments.

In certain embodiments, the AAV particles and methods of the present disclosure may be used to treat subjects suffering from SMA and related diseases and ailments. In certain embodiments, methods of the present disclosure may be used to treat subjects suspected of developing SMA or related diseases and ailments.

AAV particles, pharmaceutical formulations, and methods of using the viral particles described in the present disclosure may be used to prevent, manage and/or treat SMA and related diseases and ailments.

### Alzheimer's Disease

Alzheimer's Disease (AD) is a debilitating neurodegenerative disease and the most common form of dementia affecting the memory, thinking and behavior. Typical early symptom is difficulty of remembering newly learned information. As the disease advances, symptoms include disorientation, changes in sleep, changes in mood and behavior, confusion, unfound suspicions and eventually difficulty to speak, swallow and walk. AD currently afflicts more than 35 million people worldwide, with that number expected to double in coming decades.

As of today, no cure or prevention therapy for AD has been identified. Drug therapy to treat memory loss, behavioral changes, and sleep changes, and to slow down the progression of AD are available. However, these symptomatic treatments do not address the underlying pathophysiology.

In certain embodiments, methods of the present disclosure may be used to treat subjects suffering from AD and related diseases and ailments. In certain embodiments, methods of the present disclosure may be used as a therapy for to treat subjects suspected of developing AD or related diseases and ailments.

AAV particles, pharmaceutical formulations, and methods of using the viral particles described in the present disclosure may be used to prevent, manage and/or treat AD and related diseases and ailments.

### Amyotrophic Lateral Sclerosis

Amyotrophic Lateral Sclerosis (ALS), also known as Lou Gehrig's disease or classical motor neuron disease, is a rapidly progressive and fatal neurological disease. ALS is associated with cell degeneration and death of the upper and lower motor neurons, leading to enablement of muscle movement, weakening, wasting and loss of control over voluntary muscle movement. Early symptoms include muscle weakness of hands, legs and swallowing muscles, eventually progressing to inability to breathe due to diaphragm failure. According to Centers for Disease Control and Prevention (CDC), ALS affects an estimated 12, 000-15, 000 individuals in the US. About 5-10% of cases are familial.

ALS, as other non-infectious neurodegenerative diseases, has been characterized by presence of misfolded proteins. Familial ALS has been associated with mutations of TAR DNA-binding protein 43 (TDP-43) and RNA-binding protein FUS/TLS. Some proteins have been identified to slow down progression of ALS, such as, but not limited, to growth factors, e.g. insulin-like growth factor 1 (IGF-1), glial cell line-derived growth factor, brain-derived growth factor, vascular endothelial growth factor and ciliary neurotrophic factor, or growth factors promoting muscle growth, e.g. myostatin.

As of today, there is no prevention or cure for ALS. FDA approved drug niluzole has been approved to prolong the life but does not have an effect on symptoms. Additionally, drugs and medical devices are available to tolerate pain and attacks associated with ALS. There remains a need for therapy affecting the underlying pathophysiology.

In certain embodiments, methods of the present disclosure may be used to treat subjects suffering from ALS and related diseases and ailments. In certain embodiments, methods of the present disclosure may be used to treat subjects suspected of developing ALS or related diseases and ailments.

AAV particles, pharmaceutical formulations, and methods of using the viral particles described in the present disclosure may be used to prevent, manage and/or treat ALS and related diseases and ailments.

### Huntington's Disease

Huntington's disease (HD) is a monogenic fatal neurodegenerative disease which is a rare, inherited disorder causing degeneration of neurons in the motor control region of the brain, as well as other areas. HD affects approximately 30,000 individuals in the US. HD is caused by mutations in the gene associated with the huntingtin (HTT) protein. The mutation causes the (CAG) blocks of DNA to repeat abnormally many times. In some embodiments, a subject has fully penetrant HD where the HTT gene has 41 or more CAG repeats (e.g., 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 or more than 90 CAG repeats). In some embodiments, a subject has incomplete penetrance where the HTT gene has between 36 and 40 CAG repeats (e.g., 36, 37, 38, 39 and 40 CAG repeats).

Huntington's Disease is known to be caused by an autosomal dominant triplet (CAG) repeat expansion which encodes poly-glutamine in the N-terminus of the huntingtin (HTT) protein. The expansion threshold for occurrence of the disease is considered to be approximately 35-40 residues. This repeat expansion results in a toxic gain of function of HTT and ultimately leads to striatal neurodegeneration which progresses to widespread brain atrophy. Medium spiny neurons of the striatum appear to be especially vulnerable in HD with up to 95% loss, whereas interneurons are largely spared.

In particular, HD is also associated with beta sheet rich aggregates in striatal neurons formed by N-terminal region of HTT. The expansions and aggregates lead to gradual loss of neurons as HD progresses. Additionally, the cell death in HD is associated with death receptor 6 (DR6) which is known to induce apoptosis. Symptoms typically appear between the ages of 35-44 and life expectancy subsequent to onset is 10-25 years. Interestingly, the length of the HTT expansion correlates with both age of onset and rate of disease progression, with longer expansions linked to greater severity of disease. In a small percentage of the HD population (˜6%), disease onset occurs from 2-20 years of age with appearance of an akinetic-rigid syndrome. These cases tend to progress faster than those of the later onset variety and have been classified as juvenile or Westphal variant HD. It is estimated that approximately 35.000-70,000 patients are currently suffering from HD in the US and Europe. Currently, only symptomatic relief and supportive therapies are available for treatment of HD, with a cure yet to be identified. Ultimately, individuals with HD succumb to other diseases (e.g., pneumonia, heart failure), choking, suffocation or other complications such as physical injury from falls.

The mechanisms by which CAG-expanded HTT results in neurotoxicity are not well understood. Huntingtin protein is expressed in all cells, though its concentration is highest in the brain. The normal function of HTT is unknown, but in the brains of HD patients, HTT aggregates into abnormal nuclear inclusions. It is now believed that it is this process of misfolding and aggregating along with the associated protein intermediates (i.e. the soluble species and toxic N-terminal fragments) that result in neurotoxicity.

Huntington's Disease has a profound impact on quality of life. Symptoms typically appear between the ages of 35-44 and life expectancy subsequent to onset is 10-25 years. In a small percentage of the HD population (˜6%), disease onset occurs prior to the age of 21 with appearance of an akinetic-rigid syndrome. These cases tend to progress faster than those of the later onset variety and have been classified as juvenile or Westphal variant HD. It is estimated that approximately 35,000-70,000 patients are currently suffering from HD in the US and Europe. Currently, only symptomatic relief and supportive therapies are available for treatment of HD, with a cure yet to be identified. Ultimately, individuals with HD succumb to pneumonia, heart failure or other complications such as physical injury from falls.

Symptoms of HD may include features attributed to CNS degeneration such as, but are not limited to, chorea (uncontrolled movements), dystonia, bradykinesia, incoordination, irritability and depression, problem solving difficulties, reduction in the ability of a person to function in their normal day to day life due to changes in behavior, judgment and cognition (e.g., neuropsychiatric and cognitive dysfunction), diminished speech, and difficulty swallowing, as well as features not attributed to CNS degeneration such as, but not limited to, weight loss (e.g., from difficulty swallowing food), muscle wasting, metabolic dysfunction and endocrine disturbances.

As of today, there is no therapy to cure, or prevent the progression of the disease. Drug therapies available are aimed at management of the symptoms. For example, FDA has approved tetrabenezine to be prescribed for prevention of chorea. Additionally, e.g., antipsychotic drugs may help to control delusions, hallucinations and violent outbursts. There remains a need for therapy affecting the underlying pathophysiology, such as antibody therapies targeting the HTT protein, DR6 protein, and/or other HD associated proteins.

The adeno-associated virus (AAV) is a member of the parvovirus family and has emerged as an attractive vector for gene therapy in large part because this virus is apparently non-pathogenic; in fact. AAV has not been associated with any human disease. Further appeal is due to its ability to transduce dividing and non-diving cells (including efficient transduction of neurons), diminished proinflammatory and immune responses in humans, inability to autonomously replicate without a helper virus (AAV is a helper-dependent DNA parvovirus which belongs to the genus Dependovirus), and its long-term gene expression. Although over 10 recombinant AAV serotypes (rAAV) have been engineered into vectors, rAAV2 is the most frequently employed serotype for gene therapy trials. Additional rAAV serotypes have been developed and tested in animal models that are more efficient at neuronal transduction.

Studies in animal models of HD have suggested that phenotypic reversal is feasible, for example, subsequent to gene shut off in regulated-expression models. In a mouse model allowing shut off of expression of a 94-polyglutamine repeat HTT protein, not only was the clinical syndrome reversed but also the intracellular aggregates were resolved. Further, animal models in which silencing of HTT was tested, demonstrated promising results with the therapy being both well tolerated and showing potential therapeutic benefit. These findings indicate that HTT silencing may serve as a potential therapeutic target for treatment of HD.

Model systems for studying Huntington's Disease which may be used with the modulatory polynucleotides and AAV particles described herein include, but are not limited to, cell models (e.g., primary neurons and induced pluripotent stem cells), invertebrate models (e.g., drosophila or caenorhabditis elegans), mouse models (e.g., YAC128 mouse model; R6/2 mouse model; BAC and knock-in mouse model), rat models (e.g., BAC) and large mammal models (e.g., mini-pigs, pigs, sheep, or monkeys).

Disclosed herein are methods for treating Huntington's Disease (HD) associated with HTT protein in a subject in need of treatment. The method optionally comprises administering to the subject a therapeutically effective amount of a composition comprising at least AAV particles comprising a nucleic acid sequence encoding the siRNA molecules of the present disclosure. As a non-limiting example, the siRNA molecules can silence HTT gene expression, inhibit HTT protein production, and reduce one or more symptoms of HD in the subject such that HD is therapeutically treated.

In some embodiments. AAV particles of the present disclosure and formulations thereof, may be used to inhibit or prevent the expression of CAG-expanded HTT in a subject (e.g., subjects diagnosed with or showing signs of HD) for treatment of HD. In some embodiments, AAV particles of the present disclosure and formulations thereof, may be used to targeting HIT mRNA for the treatment of HD. The AAV particles may include modulatory polynucleotides encoding double stranded RNA (dsRNA) constructs and siRNA constructs.

In certain embodiments, methods of the present disclosure may be used to treat subjects suffering from HD and related diseases and ailments. In certain embodiments, methods of the present disclosure may be used to treat subjects suspected of developing HD or related diseases and ailments.

AAV particles, pharmaceutical formulations, and methods of using the viral particles described in the present disclosure may be used to prevent, manage and/or treat HD and related diseases and ailments.

In some embodiments, the AAV particles described herein may be used to reduce the amount of HTT in a subject in need thereof and thus provides a therapeutic benefit as described herein.

Described herein are compositions, methods, processes, kits and/or devices for the administration of AAV particles comprising modulatory polynucleotides encoding siRNA molecules for the treatment, prophylaxis, palliation and/or amelioration of Huntington's Disease (HD) related symptoms and disorders.

The present disclosure provides pharmaceutical compositions for use in the treatment of Huntington's Disease (HD) comprising AAV particles comprising modulatory polynucleotides (e.g., siRNA) targeting HTT mRNA in a pharmaceutically acceptable formulation.

In some embodiments, the AAV particle comprises an AAV viral genome comprising a polynucleotide sequence selected from the group consisting of SEQ ID NOs: 41-82 or variants having at least 95% identity thereof. In some embodiments, the polynucleotide sequence is SEQ ID NO: 41, or variants having at least 95% identity thereof.

In some embodiments, the AAV particle may comprise a serotype such as, but not limited to, any of the serotypes listed herein. In some embodiments, the AAV particle comprises an AAV1 serotype.

In some embodiments, the concentration of the AAV particle in the pharmaceutical composition is no more than 5×10¹³ VG/mL. In some embodiments, the concentration of the AAV particle is from 2.5×10¹³ to 3×10¹³ VG/mL. In some embodiments, the concentration of the AAV particle is from 5×10¹³ to 1×10¹³ VG/mL. In some embodiments, the concentration of the AAV particle is 2.7×10¹³ VG/mL. In some embodiments, the concentration of the AAV particle is 2.7×10¹² VG/mL.

In some embodiments, the pharmaceutically acceptable formulation is an aqueous solution comprising a) one or more salts; b) at least one disaccharide; and c) a buffering agent.

In some embodiments, the one or more salts may include sodium chloride, potassium chloride, and/or potassium phosphate, or a combination thereof.

In some embodiments, the salts may include sodium chloride. The concentration of sodium chloride in the formulation may be from 80 to 220 mM. The concentration of the sodium chloride may be from 85 to 110 mM. In some embodiments, the concentration of the sodium chloride is 95 mM. In some embodiments, the concentration of the sodium chloride is 100 mM.

In some embodiments, the salts may include potassium chloride. The concentration of potassium chloride may be from 1 to 3 mM. In some embodiments, the concentration of potassium chloride is from 1.5 mM.

In some embodiments, the salts may include potassium phosphate. The concentration of potassium phosphate may be from 1 to 3 mM. In some embodiments, the concentration of potassium phosphate may be 1.5 mM.

In some embodiments, the salts may include sodium chloride and potassium chloride.

In some embodiments, the salts may include sodium chloride and potassium phosphate.

In some embodiments, the salts may include sodium chloride, potassium chloride and potassium phosphate.

In some embodiments, the disaccharide may be include at least one selected from the group consisting of sucrose, lactulose, lactose, maltose, trehalose, cellobiose, chitobiose, kojibiose, nigerose, isomaltose, β,β-trehalose, α,β-trehalose, sophorose, laminaribiose, gentiobiose, turanose, maltulose, palatinose, gentiobiulose, mannobiose, melibiose, melibiulose, rutinose, rutinulose, and xylobiose.

In some embodiments, the disaccharide includes sucrose. The concentration of the sucrose may be from 5 to 9% by weight relative to the total volume of the formulation. In some embodiments, the concentration of the sucrose may be 5% by weight relative to the total volume of the formulation. In some embodiments, the concentration of the sucrose may be 7% by weight relative to the total volume of the formulation.

In some embodiments, the buffering agent may include any one selected from a group consisting of Tris HCl, Tris base, sodium phosphate, potassium phosphate, histidine, boric acid, citric acid, glycine, HEPES (4-(2-hydroxyethyl)-1-piperazineethanesulfonic acid), and MOPS (3-(N-morpholino)propanesulfonic acid).

In some embodiments, the buffering agent provides a pH from 7.2 to 8.2 at 5° C. In some embodiments, the buffering agent is at a concentration of 1-20 mM. In some embodiments, the buffering agent is at a concentration of 10 mM.

In some embodiments, the buffering agent is sodium phosphate and the pH is from 7.2 to 7.6 at 5° C.

In some embodiments, the buffering agent is Tris base and is adjusted with hydrochloric acid to a pH from 7.8 to 8.2 at 5° C.

In some embodiments, the buffering agent is Tris base and is adjusted with hydrochloric acid to a pH from 7.3 to 7.7 at 5° C.

In some embodiments, the pharmaceutically acceptable formulation further comprises a surfactant.

In some embodiments, the surfactant may be Poloxamer 188 (e.g., Pluronic® F-68). The concentration of Poloxamer 188 may be from 0.0001%-0.01% by weight (mg/L) relative to the total volume of the formulation. In some embodiments, the concentration of Poloxamer 188 is 0.001% by weight relative to the total volume of the formulation.

In some embodiments, the formulation has an osmolality of 400 to 500 mOsm/kg. In some embodiments, the osmolality may be from 400 to 480 mOsm/kg.

Further provided herein are methods of treating Huntington's Disease in a subject, by administering to the subject a therapeutically effective amount of the pharmaceutical composition described herein. In some embodiments, the pharmaceutical composition may be administered via infusion into the putamen and thalamus of the subject. The infusion may be independently bilateral or unilateral into the putamen and thalamus. The pharmaceutical composition may be administered using magnetic resonance imaging (MRI)-guided convection enhanced delivery (CED). In some embodiments, dose volumes may be administered into infusion site using ascending infusion rates.

In some embodiments, the volume of the pharmaceutical composition administered to the putamen may be no more than 1500 μL/hemisphere. In some embodiments, the volume of the pharmaceutical composition administered to the putamen may be from 300-1500 μL/hemisphere. In some embodiments, the volume of the pharmaceutical composition administered to the putamen may be 900 μL/hemisphere. In some embodiments, the dose administered to the putamen may be between 8×10¹¹ to 4×10¹³VG/hemisphere.

In some embodiments, the volume of the pharmaceutical composition administered to the thalamus may be no more than 2500 μL/hemisphere. The volume of the pharmaceutical composition administered to the thalamus may be from 1300-2500 μL/hemisphere. In some embodiments, the volume of the pharmaceutical composition administered to the thalamus may be 1700 μL/hemisphere. In some embodiments, dose administered to the thalamus may be between 3.5×10¹² to 6.8×10¹³VG/hemisphere.

In some embodiments, the total dose administered to the subject may be between 8.6×10¹² to 2×10¹⁴ VG.

In some embodiments, the methods described herein inhibit or suppress the expression of the Huntingtin (HTT) gene in the striatum of the subject. In some embodiments, the expression of the HTT gene is inhibited or suppressed in the putamen. Expression of the HTT gene may be inhibited or suppressed in one or more medium spiny neurons, and/or astrocytes in the putamen. In some embodiments, the expression of the HTT gene is inhibited or suppressed in the caudate. Expression of the HTT gene may be reduced by at least 30% in the putamen. In some embodiments, expression of HTT in the putamen may be reduced by 40-70%. In some embodiments, expression of HTT in the putamen may be reduced by 50-80%. Expression of the HTT gene may be reduced by at least 30% in the caudate. In some embodiments, expression of HTT in the caudate may be reduced by 40-70%. In some embodiments, expression of HTT in the caudate may be reduced by 50-80%.

In some embodiments, the methods described herein inhibit or suppress the expression of the Huntingtin (HTT) gene in the thalamus of the subject. Expression of the HTT gene may be inhibited or suppressed in one or more thalamic neurons, and/or astrocytes in the thalamus. Expression of the HTT gene may be reduced by at least 30% in the thalamus. In some embodiments, expression of HTT in the thalamus may be reduced by 40-80%.

In some embodiments, the methods described herein inhibit or suppress the expression of the Huntingtin (HTT) gene in the cerebral cortex of the subject. Expression of the HTT gene may be inhibited or suppressed in the primary motor and somatosensory cortex. Expression of the HIT gene is inhibited or suppressed in the pyramidal neurons of primary motor and somatosensory cortex. The expression of the HTT gene is reduced by at least 20%. In some embodiments, the expression of HTT is reduced by 30-70%.

In some embodiments, the methods described herein inhibit or suppress the expression of the Huntingtin (HTT) gene in both the striatum and cerebral cortex of the subject.

siRNA Molecules Targeting HTT

In some embodiments, modulatory polynucleotides, e.g., RNA or DNA molecules, may be used to treat neurodegenerative disease, in particular, Huntington's Disease (HD). As a non-limiting example, RNAi molecules which were designed to target against a nucleic acid sequence that encodes poly-glutamine repeat proteins which cause poly-glutamine expansion diseases such as Huntington's Disease, are described in U.S. Pat. Nos. 9,169,483 and 9,181,544 and International Patent Publication No. WO2015179525, the content of each of which is herein incorporated by reference in their entirety. U.S. Pat. Nos. 9,169,483 and 9,181,544 and International Patent Publication No. WO2015179525 each provide isolated RNA duplexes comprising a first strand of RNA (e.g., 15 contiguous nucleotides) and second strand of RNA (e.g., complementary to at least 12 contiguous nucleotides of the first strand) where the RNA duplex is about 15 to 30 base pairs in length. The first strand of RNA and second strand of RNA may be operably linked by an RNA loop (˜4 to 50 nucleotides) to form a hairpin structure which may be inserted into an expression cassette. Non-limiting examples of loop portions include SEQ ID NO: 9-14 of U.S. Pat. No. 9,169,483, the content of which is herein incorporated by reference in its entirety. Non-limiting examples of strands of RNA which may be used, either full sequence or part of the sequence, to form RNA duplexes include SEQ ID NOs: 1-8 of U.S. Pat. No. 9,169,483 and SEQ ID NOs: 1-11, 33-59, 208-210, 213-215 and 218-221 of U.S. Pat. No. 9,181,544, the contents of each of which is herein incorporated by reference in its entirety. Non-limiting examples of RNAi molecules include SEQ ID NOs: 1-8 of U.S. Pat. No. 9,169,483, SEQ ID NOs: 1-11, 33-59, 208-210, 213-215 and 218-221 of U.S. Pat. No. 9,181,544 and SEQ ID NOs: 1, 6, 7, and 35-38 of International Patent Publication No. WO2015179525, the contents of each of which is herein incorporated by reference in their entirety.

In some embodiments, small interfering RNA (siRNA) duplexes (and modulatory polynucleotides encoding them) that target HTT mRNA to interfere with HTT gene expression and/or HTT protein production are included in AAV particles and formulations thereof in the present invention.

Some guidelines for designing siRNAs have been proposed in the art. These guidelines generally recommend generating a 19-nucleotide duplexed region, symmetric 2-3 nucleotide 3′overhangs, 5′-phosphate and 3′-hydroxyl groups targeting a region in the gene to be silenced. Other rules that may govern siRNA sequence preference include, but are not limited to, (i) A/U at the 5′ end of the antisense strand; (ii) G/C at the 5′ end of the sense strand; (iii) at least five A/U residues in the 5′ terminal one-third of the antisense strand; and (iv) the absence of any GC stretch of more than 9 nucleotides in length. In accordance with such consideration, together with the specific sequence of a target gene, highly effective siRNA molecules essential for suppressing mammalian target gene expression may be readily designed.

According to the present disclosure, siRNA molecules (e.g., siRNA duplexes or encoded dsRNA) that target the HTT gene are designed. Such siRNA molecules can specifically, suppress HTT gene expression and protein production. In some aspects, the siRNA molecules are designed and used to selectively “knock out” HIT gene variants in cells, i.e., mutated HTT transcripts that are identified in patients with HD disease. In some aspects, the siRNA molecules are designed and used to selectively “knock down” HTT gene variants in cells. In other aspects, the siRNA molecules are able to inhibit or suppress both the wild type and mutated HTT gene.

In some embodiments, an siRNA molecule of the present disclosure comprises a sense strand and a complementary antisense strand in which both strands are hybridized together to form a duplex structure. The antisense strand has sufficient complementarity to the HTT mRNA sequence to direct target-specific RNAi, i.e., the siRNA molecule has a sequence sufficient to trigger the destruction of the target mRNA by the RNAi machinery or process.

In some embodiments, an siRNA molecule of the present disclosure comprises a sense strand and a complementary antisense strand in which both strands are hybridized together to form a duplex structure and where the start site of the hybridization to the HTT mRNA is between nucleotide 100 and 7000 on the HIT mRNA sequence. As a non-limiting example, the start site may be between nucleotide 100-150, 150-200, 200-250, 250-300, 300-350, 350-400, 400-450, 450-500, 500-550, 550-600, 600-650, 650-700, 700-70, 750-800, 800-850, 850-900, 900-950, 950-1000, 1000-1050, 1050-1100, 1100-1150, 1150-1200, 1200-1250, 1250-1300, 1300-1350, 1350-1400, 1400-1450, 1450-1500, 1500-1550, 1550-1600, 1600-1650, 1650-1700, 1700-1750, 1750-1800, 1800-1850, 1850-1900, 1900-1950, 1950-2000, 2000-2050, 2050-2100, 2100-2150, 2150-2200, 2200-2250, 2250-2300, 2300-2350, 2350-2400, 2400-2450, 2450-2500, 2500-2550, 2550-2600, 2600-2650, 2650-2700, 2700-2750, 2750-2800, 2800-2850, 2850-2900, 2900-2950, 2950-3000, 3000-3050, 3050-3100, 3100-3150, 3150-3200, 3200-3250, 3250-3300, 3300-3350, 3350-3400, 3400-3450, 3450-3500, 3500-3550, 3550-3600, 3600-3650, 3650-3700, 3700-3750, 3750-3800, 3800-3850, 3850-3900, 3900-3950, 3950-4000, 40004050, 4050-4100, 41004150, 41504200, 4200-4250, 42504300, 43004350, 4350-4400, 4400-4450, 44504500, 4500-4550, 4550-4600, 4600-4650, 4650-4700, 4700-4750, 47504800, 48004850, 48504900, 49004950, 4950-5000, 5000-5050, 5050-5100, 5100-5150, 5150-5200, 5200-5250, 5250-5300, 5300-5350, 5350-5400, 5400-5450, 5450-5500, 5500-5550, 5550-5600, 5600-5650, 5650-5700, 5700-5750, 5750-5800, 5800-5850, 5850-5900, 5900-5950, 5950-6000, 6000-6050, 6050-6100, 6100-6150, 6150-6200, 6200-6250, 6250-6300, 6300-6350, 6350-6400, 6400-6450, 6450-6500, 6500-6550, 6550-6600, 6600-6650, 6650-6700, 6700-6750, 6750-6800, 6800-6850, 6850-6900, 6900-6950, 6950-7000, 7000-7050, 7050-7100, 7100-7150, 7150-7200, 7200-7250, 7250-7300, 7300-7350, 7350-7400, 7400-7450, 7450-7500, 7500-7550, 7550-7600, 7600-7650, 7650-7700, 7700-7750, 7750-7800, 7800-7850, 7850-7900, 7900-7950, 7950-8000, 8000-8050, 8050-8100, 8100-8150, 8150-8200, 8200-8250, 8250-8300, 8300-8350, 8350-8400, 8400-8450, 8450-8500, 8500-8550, 8550-8600, 8600-8650, 8650-8700, 8700-8750, 8750-8800, 8800-8850, 8850-8900, 8900-8950, 8950-9000, 9000-9050, 9050-9100, 9100-9150, 9150-9200, 9200-9250, 9250-9300, 9300-9350, 9350-9400, 9400-9450, 9450-9500, 9500-9550, 9550-9600, 9600-9650.9650-9700, 9700-9750, 9750-9800, 9800-9850, 9850-9900, 9900-9950, 9950-10000, 10000-10050, 10050-10100, 10100-10150, 10150-10200, 10200-10250, 10250-10300, 10300-10350, 10350-10400, 10400-10450, 10450-10500, 10500-10550, 10550-10600, 10600-10650, 10650-10700, 10700-10750, 10750-10800, 10800-10850, 10850-10900, 10900-10950, 10950-11000, 11050-11100, 11100-11150, 11150-11200, 11200-11250, 11250-11300, 11300-11350, 11350-11400, 11400-11450, 11450-11500, 11500-11550, 11550-11600, 11600-11650, 11650-11700, 11700-11750, 11750-11800, 11800-11850, 11850-11900, 11900-11950, 11950-12000, 12000-12050, 12050-12100, 12100-12150, 12150-12200, 12200-12250, 12250-12300, 12300-12350, 12350-12400, 12400-12450, 12450-12500, 12500-12550, 12550-12600, 12600-12650, 12650-12700, 12700-12750, 12750-12800, 12800-12850, 12850-12900, 12900-12950, 12950-13000, 13050-13100, 13100-13150, 13150-13200, 13200-13250, 13250-13300, 13300-13350, 13350-13400, 13400-13450, and 13450-13500 on the HTT mRNA sequence. As yet another non-limiting example, the start site may be nucleotide 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 715, 716, 717, 718, 719, 720, 721, 722, 723, 724, 725, 875, 876, 877, 878, 879, 880, 881, 882, 883, 884, 885, 886, 887, 888, 889, 890, 891, 892, 893, 894, 895, 896, 897, 898, 899, 900, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1660, 1661, 1662, 1663, 1664, 1665, 1666, 1667, 1668, 1669, 1670, 1671, 1672, 1673, 1674, 1675, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2097, 2098, 2099, 2100, 2580, 2581, 2582, 2583, 2584, 2585, 2586, 2587, 2588, 2589, 2590, 2591, 2592, 2593, 2594, 2595, 2596, 2597, 2598, 2599, 2600, 2601, 2602, 2603, 2604, 2605, 4525, 4526, 4527, 4528, 4529, 4530, 4531, 4532, 4533, 4534, 4535, 4536, 4537, 4538, 4539, 4540, 4541, 4542, 4543, 4544, 4545, 4546, 4547, 4548, 4549, 4550, 4575, 4576, 4577, 4578, 4579, 4580, 4581, 4582, 4583, 4584, 4585, 4586, 4587, 4588, 4589, 4590, 4591, 4592, 4593, 4594, 4595, 4596, 4597, 4598, 4599, 4600, 4850, 4851, 4852, 4853, 4854, 4855, 4856, 4857, 4858, 4859, 4860, 4861, 4862, 4863, 4864, 4865, 4866, 4867, 4868, 4869, 4870, 4871, 4872, 4873, 4874, 4875, 4876, 4877, 4878, 4879, 4880, 4881, 4882, 4883, 4884, 4885, 4886, 4887, 4888, 4889, 4890, 4891, 4892, 4893, 4894, 4895, 4896, 4897, 4898, 4899, 4900, 5460, 5461, 5462, 5463, 5464, 5465, 5466, 5467, 5468, 5469, 5470, 5471, 5472, 5473, 5474, 5475, 5476, 5477, 5478, 5479, 5480, 6175, 6176, 6177, 6178, 6179, 6180, 6181, 6182, 6183, 6184, 6185, 6186, 6187, 6188, 6189, 6190, 6191, 6192, 6193, 6194, 6195, 6196, 6197, 6198, 6199, 6200, 6315, 6316, 6317, 6318, 6319, 6320, 6321, 6322, 6323, 6324, 6325, 6326, 6327, 6328, 6329, 6330, 6331, 6332, 6333, 6334, 6335, 6336, 6337, 6338, 6339, 6340, 6341, 6342, 6343, 6344, 6345, 6600, 6601, 6602, 6603, 6604, 6605, 6606, 6607, 6608, 6609, 6610, 6611, 6612, 6613, 6614, 6615, 6725, 6726, 6727, 6728, 6729, 6730, 6731, 6732, 6733, 6734, 6735, 6736, 6737, 6738, 6739, 6740, 6741, 6742, 6743, 6744, 6745, 6746, 6747, 6748, 6749, 6750, 6751, 6752, 6753, 6754, 6755, 6756, 6757, 6758, 6759, 6760, 6761, 6762, 6763, 6764, 6765, 6766, 6767, 6768, 6769, 6770, 6771, 6772, 6773, 6774, 6775, 7655, 7656, 7657, 7658, 7659, 7660, 7661, 7662, 7663, 7664, 7665, 7666, 7667, 7668, 7669, 7670, 7671, 7672, 8510, 8511, 8512, 8513, 8514, 8515, 8516, 8715, 8716, 8717, 8718, 8719, 8720, 8721, 8722, 8723, 8724, 8725, 8726, 8727, 8728, 8729, 8730, 8731, 8732, 8733, 8734, 8735, 8736, 8737, 8738, 8739, 8740, 8741, 8742, 8743, 8744, 8745, 9250, 9251, 9252, 9253, 9254, 9255, 9256, 9257, 9258, 9259, 9260, 9261, 9262, 9263, 9264, 9265, 9266, 9267, 9268, 9269, 9270, 9480, 9481, 9482, 9483, 9484, 9485, 9486, 9487, 9488, 9489, 9490, 9491, 9492, 9493, 9494, 9495, 9496, 9497, 9498, 9499, 9500, 9575, 9576, 9577, 9578, 9579, 9580, 9581, 9582, 9583, 9584, 9585, 9586, 9587, 9588, 9589, 9590, 10525, 10526, 10527, 10528, 10529, 10530, 10531, 10532, 10533, 10534, 10535, 10536, 10537, 10538, 10539, 10540, 11545, 11546, 11547, 11548, 11549, 11550, 11551, 11552, 11553, 11554, 11555, 11556, 11557, 11558, 11559, 11560, 11875, 11876, 11877, 11878, 11879, 11880, 11881, 11882, 11883, 11884, 11885, 11886, 11887, 11888, 11889, 11890, 11891, 11892, 11893, 11894, 11895, 11896, 11897, 11898, 11899, 11900, 11915, 11916, 11917, 11918, 11919, 11920, 11921, 11922, 11923, 11924, 11925, 11926, 11927, 11928, 11929, 11930, 11931, 11932, 11933, 11934, 11935, 11936, 11937, 11938, 11939, 11940, 13375, 13376, 13377, 13378, 13379, 13380, 13381, 13382, 13383, 13384, 13385, 13386, 13387, 13388, 13389 and 13390 on the HTT mRNA sequence.

In some embodiments, the antisense strand and target mRNA sequences have 100% complementarity. The antisense strand may be complementary to any part of the target mRNA sequence.

In other embodiments, the antisense strand and target mRNA sequences comprise at least one mismatch. As a non-limiting example, the antisense strand and the target mRNA sequence have at least 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98% or 99% or at least 20-30%, 20-40%, 20-50%, 20-60%, 20-70%, 20-80%, 20-90%, 20-95%, 20-99%, 30-40%, 30-50%, 30-60%, 30-70%, 30-80%, 30-90%, 30-95%, 30-99%, 40-50%, 40-60%, 40-70%, 40-80%, 40-90%, 40-95%, 40-99%, 50-60%, 50-70%, 50-80%, 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% complementarity.

In some embodiments, an siRNA or dsRNA includes at least two sequences that are complementary to each other.

According to the present disclosure, the encoded siRNA molecule has a length from about 10-50 or more nucleotides, i.e., each strand comprising 10-50 nucleotides (or nucleotide analogs). Preferably, the siRNA molecule has a length from about 15-30, e.g., 15, 16, 17, 18, 19, 20, 21, 22, 23, 24, 25, 26, 27, 28, 29, or 30 nucleotides in each strand, wherein one of the strands is sufficiently complementarity to a target region. In some embodiments, each strand of the siRNA molecule has a length from about 19 to 25, 19 to 24 or 19 to 21 nucleotides. In some embodiments, at least one strand of the siRNA molecule is 19 nucleotides in length. In some embodiments, at least one strand of the siRNA molecule is 20 nucleotides in length. In some embodiments, at least one strand of the siRNA molecule is 21 nucleotides in length. In some embodiments, at least one strand of the siRNA molecule is 22 nucleotides in length. In some embodiments, at least one strand of the siRNA molecule is 23 nucleotides in length. In some embodiments, at least one strand of the siRNA molecule is 24 nucleotides in length. In some embodiments, at least one strand of the siRNA molecule is 25 nucleotides in length.

In some embodiments, the encoded siRNA molecules of the present disclosure can be synthetic RNA duplexes comprising about 19 nucleotides to about 25 nucleotides, and two overhanging nucleotides at the 3′-end. In some aspects, the siRNA molecules may be unmodified RNA molecules. In other aspects, the siRNA molecules may contain at least one modified nucleotide, such as base, sugar or backbone modifications.

In some embodiments, the encoded siRNA molecules of the present disclosure may comprise a nucleotide sequence such as, but not limited to, an antisense (guide) sequence or a fragment or variant thereof. As a non-limiting example, the antisense sequence used in the siRNA molecule of the present disclosure is at least 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98% or 99% or at least 20-30%, 20-40%, 20-50%, 20-60%, 20-70%, 20-80%, 20-90%, 20-95%, 20-99%, 30-40%, 30-50%, 30-60%, 30-70%, 30-80%, 30-90%, 30-95%, 30-99%, 40-50%, 40-60%, 40-70%, 40-80%, 40-90%, 40-95%, 40-99%, 50-60%, 50-70%, 50-80%, 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% of a nucleotide sequence in Table 1, which includes SEQ ID NOs: 3-102, of WO2017201258, the contents of which are herein incorporated by reference in their entireties. As another non-limiting example, the antisense sequence used in the siRNA molecule of the present disclosure comprises at least 3, 4, 5, 6, 7, 8, 9, 10, 11, 12, 13, 14, 15, 16, 17, 18, 19, 20, 21 or more than 21 consecutive nucleotides of a nucleotide sequence in Table 1, which includes SEQ ID NOs: 3-102, of WO2017201258, the contents of which are herein incorporated by reference in their entireties. As yet another non-limiting example, the antisense sequence used in the siRNA molecule of the present disclosure comprises nucleotides 1 to 22, 1 to 21, 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 2 to 22, 2 to 21, 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 3 to 22, 3 to 21, 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 4 to 22, 4 to 21, 4 to 20, 4 to 19, 4 to 18, 4 to 17, 4 to 16, 4 to 15.4 to 14, 4 to 13, 4 to 12, 4 to 11.4 to 10, 4 to 9, 4 to 8, 5 to 22, 5 to 21, 5 to 20, 5 to 19, 5 to 18, 5 to 17, 5 to 16, 5 to 15, 5 to 14, 5 to 13, 5 to 12, 5 to 11, 5 to 10, 5 to 9, 5 to 8, 6 to 22, 6 to 21, 6 to 20, 6 to 19, 6 to 18, 6 to 17, 6 to 16, 6 to 15, 6 to 14, 6 to 13, 6 to 12, 6 to 11, 6 to 10, 7 to 22, 7 to 21, 7 to 20, 7 to 19, 7 to 18, 7 to 17, 7 to 16, 7 to 15, 7 to 14, 7 to 13, 7 to 12, 8 to 22, 8 to 21, 8 to 20, 8 to 19, 8 to 18, 8 to 17, 8 to 16, 8 to 15, 8 to 14, 8 to 13, 8 to 12, 9 to 22, 9 to 21, 9 to 20, 9 to 19, 9 to 18, 9 to 17, 9 to 16, 9 to 15, 9 to 14, 10 to 22, 10 to 21, 10 to 20, 10 to 19, 10 to 18, 10 to 17, 10 to 16, 10 to 15, 10 to 14, 11 to 22, 11 to 21, 11 to 20, 11 to 19, 11 to 18, 11 to 17, 11 to 16, 11 to 15, 11 to 14, 12 to 22, 12 to 21, 12 to 20, 12 to 19, 12 to 18, 12 to 17, 12 to 16, 13 to 22, 13 to 21, 13 to 20, 13 to 19, 13 to 18, 13 to 17, 13 to 16, 14 to 22, 14 to 21, 14 to 20, 14 to 19, 14 to 18, 14 to 17, 15 to 22, 15 to 21, 15 to 20, 15 to 19, 15 to 18, 16 to 22, 16 to 21, 16 to 20, 17 to 22, 17 to 21, or 18 to 22 of the sequences in Table 1, which includes SEQ ID NO: 3-102, of WO2017201258, the contents of which are herein incorporated by reference in their entireties.

In some embodiments, the encoded siRNA molecules of the present disclosure may comprise a nucleotide sequence such as, but not limited to, the sense (passenger) sequences in Table 2, which includes SEQ ID NO: 103-249, of WO2017201258, the contents of which are herein incorporated by reference in their entireties, or a fragment or variant thereof. As a non-limiting example, the sense sequence used in the siRNA molecule of the present disclosure is at least 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98% or 99% or at least 20-30%, 20-40%, 20-50%, 20-60%, 20-70%, 20-80%, 20-90%, 20-95%, 20-99%, 30-40%, 30-50%, 30-60%, 30-70%, 30-80%, 30-90%, 30-95%, 30-99%, 40-50%, 40-60%, 40-70%, 40-80%, 40-90%, 40-95%, 40-99%, 50-60%, 50-70%, 50-80%, 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% of a nucleotide sequence in Table 2, which includes SEQ ID NO: 103-249, of WO2017201258, the contents of which are herein incorporated by reference in their entireties. As another non-limiting example, the sense sequence used in the siRNA molecule of the present disclosure comprises at least 3, 4, 5, 6, 7, 8, 9, 10, 11, 12, 13, 14, 15, 16, 17, 18, 19, 20, 21 or more than 21 consecutive nucleotides of a nucleotide sequence in Table 2, which includes SEQ ID NO: 103-249, of WO2017201258, the contents of which are herein incorporated by reference in their entireties. As yet another non-limiting example, the sense sequence used in the siRNA molecule of the present disclosure comprises nucleotides 1 to 22, 1 to 21, 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 2 to 22, 2 to 21, 2 to 20, 2 to 19, 2 to 18, 2 to 17, 2 to 16, 2 to 15, 2 to 14, 2 to 13, 2 to 12, 2 to 11.2 to 10, 2 to 9, 2 to 8, 3 to 22, 3 to 21, 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 4 to 22, 4 to 21, 4 to 20, 4 to 19, 4 to 18, 4 to 17, 4 to 16, 4 to 15, 4 to 14, 4 to 13, 4 to 12, 4 to 11, 4 to 10, 4 to 9, 4 to 8, 5 to 22, 5 to 21, 5 to 20, 5 to 19, 5 to 18, 5 to 17.5 to 16, 5 to 15, 5 to 14, 5 to 13.5 to 12, 5 to 11, 5 to 10, 5 to 9, 5 to 8, 6 to 22, 6 to 21, 6 to 20, 6 to 19, 6 to 18, 6 to 17, 6 to 16, 6 to 15, 6 to 14, 6 to 13, 6 to 12, 6 to 11, 6 to 10, 7 to 22, 7 to 21, 7 to 20, 7 to 19, 7 to 18, 7 to 17, 7 to 16, 7 to 15, 7 to 14, 7 to 13, 7 to 12, 8 to 22, 8 to 21, 8 to 20, 8 to 19, 8 to 18, 8 to 17, 8 to 16, 8 to 15, 8 to 14, 8 to 13, 8 to 12, 9 to 22, 9 to 21, 9 to 20, 9 to 19, 9 to 18, 9 to 17, 9 to 16, 9 to 15, 9 to 14, 10 to 22, 10 to 21, 10 to 20, 10 to 19, 10 to 18, 10 to 17, 10 to 16, 10 to 15, 10 to 14, 11 to 22, 11 to 21, 11 to 20, 11 to 19, 11 to 18, 11 to 17, 11 to 16, 11 to 15, 11 to 14, 12 to 22, 12 to 21, 12 to 20, 12 to 19, 12 to 18, 12 to 17, 12 to 16, 13 to 22, 13 to 21, 13 to 20, 13 to 19, 13 to 18, 13 to 17, 13 to 16, 14 to 22, 14 to 21, 14 to 20, 14 to 19, 14 to 18, 14 to 17, 15 to 22, 15 to 21, 15 to 20, 15 to 19, 15 to 18, 16 to 22, 16 to 21, 16 to 20, 17 to 22, 17 to 21, or 18 to 22 of the sequences in Table 2, which includes SEQ ID NO: 103-249, of WO2017201258, the contents of which are herein incorporated by reference in their entireties.

In some embodiments, the siRNA molecules of the present disclosure may comprise the sense and antisense siRNA duplex as described in Tables 3-5, of WO2017201258, the contents of which are herein incorporated by reference in their entireties. As a non-limiting example, these siRNA duplexes may be tested for in vitro inhibitory activity on endogenous HTT gene expression. The start site for the sense and antisense sequence is compared to HTT gene sequence known as NM_002111.7 from NCBI.

The encoded siRNA duplex of the present disclosure contains an antisense strand and a sense strand hybridized together forming a duplex structure, wherein the antisense strand is complementary to the nucleic acid sequence of the targeted HTT gene, and wherein the sense strand is homologous to the nucleic acid sequence of the targeted HTT gene. In some aspects, the 5′ end of the antisense strand has a 5′ phosphate group and the 3′ end of the sense strand contains a 3′hydroxyl group. In other aspects, there are none, one or 2 nucleotide overhangs at the 3′end of each strand.

In some embodiments, the formulated AAV particles encode siRNA duplexes or dsRNA which suppress (or degrade) HTT mRNA. Accordingly, the siRNA duplexes or dsRNA can be used to substantially inhibit HTT gene expression in a cell, for example a neuron. In some aspects, the inhibition of HTT gene expression refers to an inhibition by at least about 20%, preferably by at least about 30%, 40%, 50%, 60%, 70%, 80%, 85%, 90%, 95% and 100%, or at least 20-30%, 2040%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. Accordingly, the protein product of the targeted gene may be inhibited by at least about 20%, preferably by at least about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%.

In some embodiments, the siRNA molecules comprise a miRNA seed match for the HTT located in the guide strand. In another embodiment, the siRNA molecules comprise a miRNA seed match for HTT located in the passenger strand. In yet another embodiment, the siRNA duplexes or encoded dsRNA targeting HTT gene do not comprise a seed match for HTT located in the guide or passenger strand.

In some embodiments, the siRNA duplexes or encoded dsRNA targeting HTT gene may have almost no significant full-length off target effects for the guide strand. In another embodiment, the siRNA duplexes or encoded dsRNA targeting HTT gene may have almost no significant full-length off target effects for the passenger strand. The siRNA duplexes or encoded dsRNA targeting HTT gene may have less than 1%, 2%, 3%, 4%, 5%, 6%, 7%, 8%, 9%, 10%, 11%, 12%, 13%, 14%, 15%, 20%, 25%, 30%, 35%, 40%, 45%, 50%, 1-5%, 2-6%, 3-7%, 4-8%, 5-9%, 5-10%, 6-10%, 5-15%, 5-20%, 5-25% 5-30%, 10-20%, 10-30%, 10-40%, 10-50%, 15-30%, 15-40%, 15-45%, 20-40%, 20-50%, 25-50%, 30-40%, 30-50%, 35-50%, 40-50%, 45-50% full-length off target effects for the passenger strand. In yet another embodiment, the siRNA duplexes or encoded dsRNA targeting HTT gene may have almost no significant full-length off target effects for the guide strand or the passenger strand. The siRNA duplexes or encoded dsRNA targeting HTT gene may have less than 1%, 2%, 3%, 4%, 5%, 6%, 7%, 8%, 9%, 10%,11%, 12%, 13%, 14%, 15%, 20%, 25%, 30%, 35%, 40%, 45%, 50%, 1-5%, 2-6%, 3-7%, 4-8%, 5-9%, 5-10%, 6-10%, 5-15%, 5-20%, 5-25% 5-30%, 10-20%, 10-30%, 10-40%, 10-50%, 15-30%, 15-40%, 15-45%, 20-40%, 20-50%, 25-50%, 30-40%, 30-50%, 35-50%, 40-50%, 45-50% full-length off target effects for the guide or passenger strand.

In some embodiments, the siRNA duplexes or encoded dsRNA targeting HTT gene may have high activity in vitro. In another embodiment, the siRNA molecules may have low activity in vitro. In yet another embodiment, the siRNA duplexes or dsRNA targeting the HTT gene may have high guide strand activity and low passenger strand activity in vitro.

In some embodiments, the siRNA molecules have a high guide strand activity and low passenger strand activity in vitro. The target knock-down (KD) by the guide strand may be at least 30%, 40%, 50%, 60%, 65%, 70%, 75%, 80%, 85%, 90%, 95%, 99%, 99.5% or 100%. The target knock-down by the guide strand may be 30-40%, 35-40%, 40-50%, 45-50%, 50-55%, 50-60%, 60-65%, 60-70%, 60-75%, 60-80%, 60-85%, 60-90%, 60-95%, 60-99%, 60-99.5%, 60-100%, 65-70%, 65-75%, 65-80%, 65-85%, 65-90%, 65-95%, 65-99%, 65-99.5%, 65-100%, 70-75%, 70-80%, 70-85%, 70-90%, 70-95%, 70-99%, 70-99.5%, 70-100%, 75-80%, 75-85%, 75-90%, 75-95%, 75-99%, 75-99.5%, 75-100%, 80-85%, 80-90%, 80-95%, 80-99%, 80-99.5%, 80-100%, 85-90%, 85-95%, 85-99%, 85-99.5%, 85-100%, 90-95%, 90-99%, 90-99.5%, 90-100%, 95-99%, 95-99.5%, 95-100%, 99-99.5%, 99-100% or 99.5-100%. As a non-limiting example, the target knock-down (KD) by the guide strand is greater than 70%. As a non-limiting example, the target knock-down (KD) by the guide strand is greater than 60%.

In some embodiments, the siRNA duplex is designed so there is no miRNA seed match for the sense or antisense sequence to non-HTT sequence.

In some embodiments, the IC₅₀ of the guide strand for the nearest off target is greater than 100 multiplied by the IC₅₀ of the guide strand for the on-target gene, HTT. As a non-limiting example, if the IC₅₀ of the guide strand for the nearest off target is greater than 100 multiplied by the IC₅₀ of the guide strand for the target then the siRNA molecule is said to have high guide strand selectivity for inhibiting HTT in vitro.

In some embodiments, the 5′ processing of the guide strand has a correct start (n) at the 5′ end at least 75%, 80%, 85%, 90%, 95%, 99% or 100% of the time in vitro or in vivo. As a non-limiting example, the 5′ processing of the guide strand is precise and has a correct start (n) at the 5′ end at least 99% of the time in vitro. As a non-limiting example, the 5′ processing of the guide strand is precise and has a correct start (n) at the 5′ end at least 99% of the time in vivo. As a non-limiting example, the 5′ processing of the guide strand is precise and has a correct start (n) at the 5′ end at least 90% of the time in vitro. As a non-limiting example, the 5′ processing of the guide strand is precise and has a correct start (n) at the 5′ end at least 90% of the time in vivo. As a non-limiting example, the 5′ processing of the guide strand is precise and has a correct start (n) at the 5′ end at least 85% of the time in vitro. As a non-limiting example, the 5′ processing of the guide strand is precise and has a correct start (n) at the 5′ end at least 85% of the time in vivo.

In some embodiments, the guide to passenger (G:P) (also referred to as the antisense to sense) strand ratio expressed is 1:10, 1:9, 1:8, 1:7, 1:6, 1:5, 1:4, 1:3, 1:2, 1:1, 2:10, 2:9, 2:8, 2:7, 2:6, 2:5, 2:4, 2:3, 2:2, 2:1, 3:10, 3:9, 3:8, 3:7, 3:6, 3:5, 3:4, 3:3, 3:2, 3:1, 4:10, 4:9, 4:8, 4:7, 4:6, 4:5, 4:4, 4:3, 4:2, 4:1, 5:10, 5:9, 5:8.5:7, 5:6, 5:5, 5:4, 5:3.5:2.5:1, 6:10, 6:9, 6:8, 6:7, 6:6, 6:5, 6:4, 6:3, 6:2, 61, 7:10, 7:9, 7:8, 7:7, 7:6, 7:5, 74, 7:3, 7:2.7:1, 8:10, 8:9, 8:8, 8:7, 8:6, 8:5, 8:4, 8:3, 8:2, 8:1, 9:10, 9:9, 9:8, 9:7, 9:6, 9:5, 9:4, 9:3, 9:2, 9:1, 10:10, 10:9, 10:8, 10:7, 10:6, 10:5, 10:4, 10:3, 10:2, 10:1, 1:99, 5:95, 10:90, 15:85, 20:80, 25:75, 30:70, 35:65, 40:60, 45:55, 50:50, 55:45, 60:40, 65:35, 70:30, 75:25, 80:20, 85:15, 90:10, 95:5, or 99:1 in vitro or in vivo. The guide to passenger ratio refers to the ratio of the guide strands to the passenger strands after intracellular processing of the pri-microRNA. For example, a 80:20 guide-to-passenger ratio would have 8 guide strands to every 2 passenger strands processed from the precursor. As a non-limiting example, the guide-to-passenger strand ratio is 8:2 in vitro. As a non-limiting example, the guide-to-passenger strand ratio is 8:2 in vivo. As a non-limiting example, the guide-to-passenger strand ratio is 9:1 in vitro. As a non-limiting example, the guide-to-passenger strand ratio is 9:1 in vivo.

In some embodiments, the guide to passenger (G:P) (also referred to as the antisense to sense) strand ratio expressed is greater than 1.

In some embodiments, the guide to passenger (G:P) (also referred to as the antisense to sense) strand ratio expressed is greater than 2.

In some embodiments, the guide to passenger (G:P) (also referred to as the antisense to sense) strand ratio expressed is greater than 5.

In some embodiments, the guide to passenger (G:P) (also referred to as the antisense to sense) strand ratio expressed is greater than 10.

In some embodiments, the guide to passenger (G:P) (also referred to as the antisense to sense) strand ratio expressed is greater than 20.

In some embodiments, the guide to passenger (G:P) (also referred to as the antisense to sense) strand ratio expressed is greater than 50.

In some embodiments, the guide to passenger (G:P) (also referred to as the antisense to sense) strand ratio expressed is at least 3:1.

In some embodiments, the guide to passenger (G:P) (also referred to as the antisense to sense) strand ratio expressed is at least 5:1.

In some embodiments, the guide to passenger (G:P) (also referred to as the antisense to sense) strand ratio expressed is at least 10:1.

In some embodiments, the guide to passenger (G:P) (also referred to as the antisense to sense) strand ratio expressed is at least 20:1.

In some embodiments, the guide to passenger (G:P) (also referred to as the antisense to sense) strand ratio expressed is at least 50:1.

In some embodiments, the passenger to guide (P:G) (also referred to as the sense to antisense) strand ratio expressed is 1:10, 1:9, 1:8, 1:7, 1:6, 1:5, 1:4, 1:3, 1:2, 1:1, 2:10, 2:9, 2:8, 2:7, 2:6, 2:5, 2:4, 2:3, 2:2, 2:1, 3:10, 3:9, 3:8, 3:7, 3:6, 3:5, 3:4, 3:3, 3:2, 3:1, 4:10, 4:9, 4:8, 4:7, 4:6, 4:5, 4:4, 4:3, 4:2, 4:1, 5:10, 5:9, 5:8, 5:7, 5:6, 5:5, 5:4, 5:3, 5:2, 5:1, 6:10, 6:9, 6:8, 6:7, 6:6, 6:5, 6:4, 6.3, 6:2, 6:1, 7:10, 7:9, 7:8, 7:7, 7:6, 7:5, 7:4, 7:3, 7:2, 7:1, 8:10, 8:9, 8:8, 8:7, 8:6, 8:5, 8:4, 8:3, 8:2, 8:1, 9:10, 9:9, 9:8, 9:7, 9:6, 9:5, 9:4, 9:3, 9:2, 9:1, 10:10, 10:9, 10:8, 10:7, 10:6, 10:5, 10:4, 10:3, 10:2, 10:1, 1:99, 5:95, 10:90, 15:85, 20:80, 25:75, 30:70, 35:65, 40:60, 45:55, 50:50, 55:45, 60:40, 65:35, 70:30, 75:25, 80:20, 85:15, 90:10, 95:5, or 99:1 in vitro or in vivo. The passenger to guide ratio refers to the ratio of the passenger strands to the guide strands after the intracellular processing of the pri-microRNA. For example, a 80:20 of passenger-to-guide ratio would have 8 passenger strands to every 2 guide strands processed from the precursor. As a non-limiting example, the passenger-to-guide strand ratio is 80:20 in vitro. As a non-limiting example, the passenger-to-guide strand ratio is 80:20 in vivo. As a non-limiting example, the passenger-to-guide strand ratio is 8:2 in vitro. As a non-limiting example, the passenger-to-guide strand ratio is 8:2 in vivo. As a non-limiting example, the passenger-to-guide strand ratio is 9:1 in vitro. As a non-limiting example, the passenger-to-guide strand ratio is 9:1 in vivo.

In some embodiments, the passenger to guide (P:G) (also referred to as the sense to antisense) strand ratio expressed is greater than 1.

In some embodiments, the passenger to guide (P:G) (also referred to as the sense to antisense) strand ratio expressed is greater than 2.

In some embodiments, the passenger to guide (P:G) (also referred to as the sense to antisense) strand ratio expressed is greater than 5.

In some embodiments, the passenger to guide (P:G) (also referred to as the sense to antisense) strand ratio expressed is greater than 10.

In some embodiments, the passenger to guide (P:G) (also referred to as the sense to antisense) strand ratio expressed is greater than 20.

In some embodiments, the passenger to guide (P:G) (also referred to as the sense to antisense) strand ratio expressed is greater than 50.

In some embodiments, the passenger to guide (P:G) (also referred to as the sense to antisense) strand ratio expressed is at least 3:1.

In some embodiments, the passenger to guide (P:G) (also referred to as the sense to antisense) strand ratio expressed is at least 5:1.

In some embodiments, the passenger to guide (P:G) (also referred to as the sense to antisense) strand ratio expressed is at least 10:1.

In some embodiments, the passenger to guide (P:G) (also referred to as the sense to antisense) strand ratio expressed is at least 20:1.

In some embodiments, the passenger to guide (P:G) (also referred to as the sense to antisense) strand ratio expressed is at least 50:1.

In some embodiments, a passenger-guide strand duplex is considered effective when the pri- or pre-microRNAs demonstrate, but methods known in the art and described herein, greater than 2-fold guide to passenger strand ratio when processing is measured. As a non-limiting examples, the pri- or pre-microRNAs demonstrate great than 2-fold, 3-fold, 4-fold, 5-fold, 6-fold, 7-fold, 8-fold, 9-fold, 10-fold, li-fold, 12-fold. 13-fold, 14-fold, 15-fold, or 2 to 5-fold, 2 to 10-fold, 2 to 15-fold, 3 to 5-fold, 3 to 10-fold, 3 to 15-fold, 4 to 5-fold, 4 to 10-fold, 4 to 15-fold, 5 to 10-fold, 5 to 15-fold, 6 to 10-fold, 6 to 15-fold, 7 to 10-fold, 7 to 15-fold. 8 to 10-fold, 8 to 15-fold, 9 to 10-fold, 9 to 15-fold, 10 to 15-fold, 11 to 15-fold, 12 to 15-fold, 13 to 15-fold, or 14 to 15-fold guide to passenger strand ratio when processing is measured.

In some embodiments, the vector genome encoding the dsRNA comprises a sequence which is at least 60%, 65%, 70%, 75%, 80%, 85%, 90%, 95%, 99% or more than 99% of the full length of the construct. As a non-limiting example, the vector genome comprises a sequence which is at least 80% of the full-length sequence of the construct.

In some embodiments, the siRNA molecules may be used to silence wild type and/or mutant HTT by targeting at least one exon on the HTT sequence. The exon may be exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, exon 31, exon 32, exon 33, exon 34, exon 35, exon 36, exon 37, exon 38, exon 39, exon 40, exon 41, exon 42, exon 43, exon 44, exon 45, exon 46, exon 47, exon 48, exon 49, exon 50, exon 51, exon 52, exon 53, exon 54, exon 55, exon 56, exon 57, exon 58, exon 59, exon 60, exon 61, exon 62, exon 63, exon 64, exon 65, exon 66, and/or exon 67. As a non-limiting example, the siRNA molecules may be used to silence wild type and/or mutant HTT by targeting exon 1. As another non-limiting example, the siRNA molecules may be used to silence wild type and/or mutant HTT by targeting an exon other than exon 1. As another non-limiting example, the siRNA molecules may be used to silence wild type and/or mutant HTT by targeting exon 50. As another non-limiting example, the siRNA molecules may be used to silence wild type and/or mutant HTT by targeting exon 67.

According to the present disclosure. AAV particles comprising the nucleic acids encoding the siRNA molecules targeting HTT mRNA are produced, the AAV serotypes may be any of the serotypes listed in Table 1. Non-limiting examples of the AAV serotypes include, AAV1, AAV2, AAV3, AAV4, AAV5, AAV6, AAV7, AAV8, AAV9, AAV9.47, AAV9(hu14), AAV10, AAV11, AAV12, AAVrh8, AAVrh10, AAV-DJ8, AAV-DJ, AAV-PHP.A, and/or AAV-PHP.B, and variants thereof.

### AAV Particle Comprising HTT Modulatory Polynucleotides

In some embodiments, the AAV particle comprises a viral genome with a payload region comprising a modulatory polynucleotide sequence. In such an embodiment, a viral genome encoding more than one polypeptide may be replicated and packaged into a viral particle. A target cell transduced with a viral particle comprising a modulatory polynucleotide may express the encoded sense and/or antisense sequences in a single cell.

In some embodiments, the AAV particles are useful in the field of medicine for the treatment, prophylaxis, palliation, or amelioration of neurological diseases and/or disorders.

Non-limiting examples of ITR-to-ITR sequences of AAV particles comprising a viral genome with a payload region comprising a modulatory polynucleotide with siRNA molecules targeting HTT are described in Table 3.

TABLE 3
ITR to ITR Sequences of AAV Particles
comprising HTT Modulatory Polynucleotides

| ITR to ITR | ITR to ITR |
|---|---|
| Construct Name | SEQ ID NO |
| VOYHT1 | 41 |
| VOYHT2 | 42 |
| VOYHT3 | 43 |
| VOYHT4 | 44 |
| VOYHT5 | 45 |
| VOYHT6 | 46 |
| VOYHT7 | 47 |
| VOYHT8 | 48 |
| VOYHT9 | 49 |
| VOYHT10 | 50 |
| VOYHT11 | 51 |
| VOYHT12 | 52 |
| VOYHT13 | 53 |
| VOYHT14 | 54 |
| VOYHT15 | 55 |
| VOHYT16 | 56 |
| VOYHT17 | 57 |
| VOYHT18 | 58 |
| VOYHT19 | 59 |
| VOYHT20 | 60 |
| VOYHT21 | 61 |
| VOYHT22 | 62 |
| VOYHT23 | 63 |
| VOYHT24 | 64 |
| VOYHT25 | 65 |
| VOYHT26 | 66 |
| VOYHT27 | 67 |
| VOYET28 | 68 |
| VOYHT35 | 69 |
| VOYHT36 | 70 |
| VOYHT37 | 71 |
| VOYHT38 | 72 |
| VOYHT39 | 73 |
| VOYHT40 | 74 |
| VOYHT41 | 75 |
| VOYHT42 | 76 |
| VOYHT43 | 77 |
| VOYHT44 | 78 |
| VOYHT45 | 79 |
| VOYHT46 | 80 |
| VOVHT47 | 81 |
| VOYHT48 | 82 |

In some embodiments, the AAV particle comprises a viral genome which comprises a sequence which has a percent identity to any of SEQ ID NOs: 41-82. The viral genome may have 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99% or 100% identity to any of SEQ ID NOs: 41-82. The viral genome may have 1-10%, 10-20%, 30-40%, 50-60%, 50-70%, 50-80%, 50-90%, 50-99%, 50-100%, 60-70%, 60-80%, 60-90%, 60-99%, 60-100%, 70-80%, 70-90%, 70-99%, 70-100%, 80-85%, 80-90%, 80-95%, 80-99%, 80-100%, 90-95%, 90-99%, or 90-100% to any of SEQ ID NOs: 41-82. As a non-limiting example, the viral genome comprises a sequence which as 80% identity to any of SEQ ID NOs: 41-82. As another non-limiting example, the viral genome comprises a sequence which as 85% identity to any of SEQ ID NOs: 41-82. As another non-limiting example, the viral genome comprises a sequence which as 90% identity to any of SEQ ID NOs: 41-82. As another non-limiting example, the viral genome comprises a sequence which as 95% identity to any of SEQ ID NOs: 41-82. As another non-limiting example, the viral genome comprises a sequence which as 99% identity to any of SEQ ID NOs: 41-82.

In some embodiments, the AAV particle comprises a viral genome which comprises a sequence corresponding to SEQ ID NO: 41, or variants having at least 95% identity thereof. The AAV particle may comprise an AAV1 serotype.

In some embodiments, the AAV particles comprising modulatory polynucleotide sequence which comprises a nucleic acid sequence encoding at least one siRNA molecule may be introduced into mammalian cells.

Where the AAV particle payload region comprises a modulatory polynucleotide, the modulatory polynucleotide may comprise sense and/or antisense sequences to knock down a target gene. The AAV viral genomes encoding modulatory polynucleotides described herein may be useful in the fields of human disease, viruses, infections veterinary applications and a variety of in vivo and in vitro settings.

AAV particles may be modified to enhance the efficiency of delivery. Such modified AAV particles comprising the nucleic acid sequence encoding the siRNA molecules of the present disclosure can be packaged efficiently and can be used to successfully infect the target cells at high frequency and with minimal toxicity.

In some embodiments, the AAV particle comprising a nucleic acid sequence encoding the siRNA molecules of the present disclosure may be a human serotype AAV particle. Such human AAV particle may be derived from any known serotype, e.g., from any one of serotypes AAV1-AAV11. As non-limiting examples, AAV particles may be vectors comprising an AAV1-derived genome in an AAV1-derived capsid; vectors comprising an AAV2-derived genome in an AAV2-derived capsid; vectors comprising an AAV4-derived genome in an AAV4 derived capsid; vectors comprising an AAV6-derived genome in an AAV6 derived capsid or vectors comprising an AAV9-derived genome in an AAV9 derived capsid.

In other embodiments, the AAV particle comprising a nucleic acid sequence for encoding siRNA molecules of the present disclosure may be a pseudotyped hybrid or chimeric AAV particle which contains sequences and/or components originating from at least two different AAV serotypes. Pseudotyped AAV particles may be vectors comprising an AAV genome derived from one AAV serotype and a capsid protein derived at least in part from a different AAV serotype. As non-limiting examples, such pseudotyped AAV particles may be vectors comprising an AAV2-derived genome in an AAV1-derived capsid; or vectors comprising an AAV2-derived genome in an AAV6-derived capsid: or vectors comprising an AAV2-derived genome in an AAV4-derived capsid: or an AAV2-derived genome in an AAV9-derived capsid. In like fashion, the present disclosure contemplates any hybrid or chimeric AAV particle.

In other embodiments, AAV particles comprising a nucleic acid sequence encoding the siRNA molecules of the present disclosure may be used to deliver siRNA molecules to the central nervous system (e.g., U.S. Pat. No. 6,180,613; the contents of which is herein incorporated by reference in its entirety).

In some aspects, the AAV particles comprising a nucleic acid sequence encoding the siRNA molecules of the present disclosure may further comprise a modified capsid including peptides from non-viral origin. In other aspects, the AAV particle may contain a CNS specific chimeric capsid to facilitate the delivery of encoded siRNA duplexes into the brain and the spinal cord. For example, an alignment of cap nucleotide sequences from AAV variants exhibiting CNS tropism may be constructed to identify variable region (VR) sequence and structure.

### Administration and Dosing

### Administration

In some embodiments, the AAV particle may be administered to the CNS in a therapeutically effective amount to improve function and/or survival for a subject with Huntington's Disease (HD). As a non-limiting example, the vector may be administered by direct infusion into the striatum.

In some embodiments, the AAV particle may be administered to a subject (e.g., to the CNS of a subject via intrathecal administration) in a therapeutically effective amount for the siRNA duplexes or dsRNA to target the medium spiny neurons, cortical neurons and/or astrocytes. As a non-limiting example, the siRNA duplexes or dsRNA may reduce the expression of HTT protein or mRNA. As another non-limiting example, the siRNA duplexes or dsRNA can suppress HTT and reduce HTT mediated toxicity. The reduction of HTT protein and/or mRNA as well as HTT mediated toxicity may be accomplished with almost no enhanced inflammation.

In some embodiments, the AAV particle may be administered to a subject (e.g., to the CNS of a subject) in a therapeutically effective amount to slow the functional decline of a subject (e.g., determined using a known evaluation method such as the unified Huntington's disease rating scale (UHDRS)). As a non-limiting example, the vector may be administered via intraparenchymal injection.

### Dosing

The pharmaceutical compositions of the present disclosure may be administered to a subject using any amount effective for reducing, preventing and/or treating a HTT associated disorder (e.g., Huntington' Disease (HD)). The exact amount required will vary from subject to subject, depending on the species, age, and general condition of the subject, the severity of the disease, the particular composition, its mode of administration, its mode of activity, and the like.

The compositions of the present disclosure are typically formulated in unit dosage form for ease of administration and uniformity of dosage. It will be understood, however, that the total daily usage of the compositions of the present disclosure may be decided by the attending physician within the scope of sound medical judgment. The specific therapeutic effectiveness for any particular patient will depend upon a variety of factors including the disorder being treated and the severity of the disorder: the activity of the specific compound employed; the specific composition employed: the age, body weight, general health, sex and diet of the patient; the time of administration, route of administration, and rate of excretion of the siRNA duplexes employed; the duration of the treatment; drugs used in combination or coincidental with the specific compound employed; and like factors well known in the medical arts.

In some embodiments, the age and sex of a subject may be used to determine the dose of the compositions of the present disclosure. As a non-limiting example, a subject who is older may receive a larger dose (e.g., 5-10%, 10-20%, 15-30%,20-50%, 25-50% or at least 1%, 2%, 3%, 4%, 5%, 10%, 20%, 30%, 40%, 50%, 60%, 70%, 80%, 90% or more than 90% more) of the composition as compared to a younger subject. As another non-limiting example, a subject who is younger may receive a larger dose (e.g., 5-10%, 10-20%, 15-30%, 20-50%, 25-50% or at least 1%, 2%, 3%, 4%, 5%, 10%, 20%, 30%, 40%, 50%, 60%, 70%, 80%, 90% or more than 90% more) of the composition as compared to an older subject. As yet another non-limiting example, a subject who is female may receive a larger dose (e.g., 5-10%, 10-20%, 15-30%,20-50%, 25-50% or at least 1%, 2%, 3%, 4%, 5%, 10%, 20%, 30%, 40%, 50%, 60%, 70%, 80%, 90% or more than 90% more) of the composition as compared to a male subject. As yet another non-limiting example, a subject who is male may receive a larger dose (e.g., 5-10%, 10-20%, 15-30%,20-50%, 25-50% or at least 1%, 2%, 3%, 4%, 5%, 10%, 20%, 30%, 40%, 50%, 60%, 70%, 80%, 90% or more than 90% more) of the composition as compared to a female subject

In some specific embodiments, the doses of AAV particles for delivering siRNA duplexes of the present disclosure may be adapted depending on the disease condition, the subject, and the treatment strategy.

In some embodiments, delivery of the compositions in accordance with the present disclosure to cells comprises a rate of delivery defined by [VG/hour=mL/hour*VG/mL] wherein VG is viral genomes, VG/mL is composition concentration, and mL/hour is rate of prolonged delivery.

In some embodiments, delivery of compositions in accordance with the present disclosure to cells may comprise a total concentration per subject between about 1×10⁶ VG and about 1×10¹⁶ VG. In some embodiments, delivery may comprise a composition concentration of about 1×10⁶, 2×10⁶, 3×10⁶, 4×10⁶. 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰10, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10⁴¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10², 2.1×10², 2.2×10¹², 2.3×10¹², 2.4×10¹², 2.5×10¹², 2.6×10¹², 2.7×10¹², 2.8×10¹², 2.9×10¹², 3×10¹², 3.1×10¹², 3.2×10¹², 3.3×10¹², 3.4×10¹², 3.5×10¹², 3.6×10¹², 3.7×10¹², 3.8×10¹², 3.9×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 6.1×10¹², 6.2×10¹², 6.3×10¹², 6.4×10¹², 6.5×10¹², 6.6×10¹², 6.7×10¹², 6.8×10¹², 6.9×10¹², 7×10¹², 8×10¹², 8.1×10¹², 8.2×10¹², 8.3×10¹², 8.4×10¹², 8.5×10¹², 8.6×10¹², 8.7×10¹², 8.8×10¹², 8.9×10¹², 9×10¹², 1×10¹³, 1.1×10¹³, 1.2×10¹³, 1.3×10¹³, 1.4×10¹³, 1.5×10¹³, 1.6×10¹³, 1.7×10¹³, 1.8×10¹³, 1.9×10¹³, 2×10¹³, 2.7×10¹³, 3×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/subject or VG/dose.

In some embodiments, delivery of compositions in accordance with the present disclosure to cells may comprise a total concentration per subject between about 1×10⁶ VG/kg and about 1×10¹⁶ VG/kg. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹², 3×10¹², 3.1×10¹², 3.2×10¹², 3.3×10¹², 3.4×10¹², 3.5×10¹², 3.6×10¹², 3.7×10¹², 3.8×10¹², 3.9×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 6.1×10¹², 6.2×10¹², 6.3×10¹², 6.4×10¹², 6.5×10¹², 6.6×10¹², 6.7×10¹², 6.8×10¹², 6.9×10¹², 7×10¹², 8×10¹², 8.1×10¹², 8.2×10¹², 8.3×10¹², 8.4×10¹², 8.5×10¹², 8.6×10¹², 8.7×10¹², 8.8×10¹², 8.9×10¹², 9×10¹², 1×10¹², 1×10¹³, 1.2×10¹³, 0.3×10¹³, 1.4×10¹³, 1.5×10¹³, 1.6×10¹³, 1.7×10¹³, 1.8×10¹³, 1.9×10¹³, 2×10¹³, 2.7×10¹³, 3×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/kg.

In some embodiments, delivery of the compositions in accordance with the present disclosure to cells may comprise a total concentration between about 1×10⁶ VG/mL and about 1×10¹⁶ VG/mL. In some embodiments, delivery may comprise a composition concentration of about 1×10⁶, 2×10⁶, 3×10⁶. 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰10, 7×10¹⁰10, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 3×10¹¹, 4×10¹¹, 5×10¹¹, 6×10¹¹, 7×10¹¹, 8×10¹¹, 9×10¹¹, 1×10¹², 1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹², 3×10¹², 3.1×10¹², 3.2×10¹². 3.3×10¹², 3.4×10¹², 3.5×10¹², 3.6×10¹², 3.7×10¹², 3.8×10¹², 3.9×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 6.1×10¹², 6.2×10¹², 6.3×10¹², 6.4×10¹², 6.5×10¹², 6.6×10¹², 6.7×10¹², 6.8×10¹², 6.9×10¹², 7×10¹², 8×10¹², 9×10¹², 1×10¹³, 1.1×10¹³, 1.2×10¹³, 1.3×10¹³, 1.4×10¹³, 1.5×10¹³, 1.6×10¹³, 1.7×10¹³, 1.8×10¹³, 1.9×10¹³, 2×10¹³, 2.7×10¹³, 3×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/mL.

In some embodiments, the compositions in accordance with the present disclosure to be delivered may comprise a concentration between 9×10¹¹ VG/mL-2.7×10¹³ VG/mL. In some embodiments, the compositions in accordance with the present disclosure to be delivered may comprise a concentration of 2.7×10¹³ VG/mL.

In some embodiments, delivery of the compositions in accordance with the present disclosure to cells may comprise a total concentration between about 1×10⁶ total capsid/mL and about 1×10¹⁶ total capsid/mL. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹², 3×10¹², 3.1×10¹², 3.2×10¹², 3.3×10¹², 3.4×10¹², 3.5×10¹², 3.6×10¹², 3.7×10¹², 3.8×10¹², 3.9×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 8×10¹², 9×10¹², 1×10¹³, 2×10¹³, 2.7×10¹³, 3×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ total capsid/mL.

In certain embodiments, the desired siRNA duplex dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. As used herein, a “split dose” is the division of single unit dose or total daily dose into two or more doses, e.g., two or more administrations of the single unit dose. As used herein, a “single unit dose” is a dose of any modulatory polynucleotide therapeutic administered in one dose/at one time/single route/single point of contact, i.e., single administration event. As used herein, a “total daily dose” is an amount given or prescribed in a 24-hour period. It may be administered as a single unit dose. In some embodiments, the AAV particles comprising the modulatory polynucleotides of the present disclosure are administered to a subject in split doses. They may be formulated in buffer only or in a formulation described herein.

In some embodiments, the dose, concentration and/or volume of the composition described herein may be adjusted depending on the contribution of the caudate or putamen to cortical and subcortical distribution after administration. The administration may be intracerebroventricular, intraputamenal, intrathalamic, intraparenchymal, subpial, and/or intrathecal administration.

In some embodiments, the dose, concentration and/or volume of the composition described herein may be adjusted depending on the cortical and neuraxial distribution following administration by intracerebroventricular, intraputamenal, intrathalamic, intraparenchymal, subpial, and/or intrathecal delivery.

The volume of the pharmaceutical compositions to be administered may be determined based on the subject, the volume of the targeted structure, and/or the dose of the composition. In some embodiments, the subject is a primate. In some embodiments, the subject is a non-human primate. In some embodiments, the subject is a human.

In some embodiments, the volume of the pharmaceutical composition to be infused to a putamen or thalamus in a subject may be between about 1-3000 μL per side. In some embodiments, the volume of the composition to be infused to a putamen or thalamus may be about 10 μl, 25 μl, 50 μl, 75 μl. 100 μl, 125 μl, 150 μl, 175 μl. 200 μl, 225 μl, 250 μl, 275 μl, 300 μl. 325 μl, 350 μl, 375 μl, 400 μl. 425 μl, 450 μl, 475 μl, 500 μl, 525 μl, 550 μl, 575 μl, 600 μl, 625 μl, 650 μl, 675 μl, 700 μl, 725 μl, 750 μl, 775 μl, 800 μl, 825 μl, 850 μl, 875 μl, 900 μl, 925 μl, 950 μl, 975 μl, 1000 μl, 1025 μl. 1050 μl, 1075 μl, 1100 μl, 1125 μl, 1150 μl. 1175 μl, 1200 μl, 1225 μl, 1250 μl. 1275 μl, 1300 μl, 1325 μl, 1350 μl, 1375 μl. 1400 μl, 1425 μl, 1450 μl, 1475 μl, 1500 μl, 1600 μl, 1700 μl, 1800 μl, 1900 μl, 2000 μl, 2250 μl, 2500 μl, 2750 μl, or 3000 μl per side.

In some embodiments, the pharmaceutical composition described herein is administered to a subject which is a non-human primate. In some embodiments, the volume of the composition to be infused to the putamen in a non-human primate is 50-150 μL per side. In some embodiments, the volume of the composition to be infused to the putamen in a non-human primate is 100-200 μL per side. In some embodiments, the volume of the composition to be infused to the putamen in a non-human primate is 175-525 μL per side.

In some embodiments, the volume of the composition to be infused to the thalamus in a non-human primate is 70-250 μL per side. In some embodiments, the volume of the composition to be infused to the thalamus in a non-human primate is 200-300 μL per side. In some embodiments, the volume of the composition to be infused to the thalamus in a non-human primate is 450-1500 μL per side.

In some embodiments, the pharmaceutical composition described herein is administered to a subject which is a human. In some embodiments, the volume of the pharmaceutical composition administered to the putamen in a human may be no more than 2000 μL/hemisphere. In some embodiments, the volume of the composition to be infused to the putamen in a human is no more than 1500 μL/hemisphere per side.

In some embodiments, the volume of the composition to be infused to the putamen in a human is 300-1500 μL per side. In some embodiments, the volume of the composition to be infused to the putamen in a human may be about 300 μl, 325 μl, 350 μl, 375 μl, 400 μl, 425 μl, 450 μl, 475 μl, 500 μl, 525 μl, 550 μl, 575 μl, 600 μl, 625 μl, 650 μl, 675 μl, 700 μl, 725 μl. 750 μl, 775 μl, 800 μl, 825 μl. 850 μl, 875 μl, 900 μl, 925 μl. 950 μl, 975 μl, 1000 μl. 1025 μl, 1050 μl, 1075 μl, 1100 μl, 1125 μl, 1150 μl, 1175 μl, 1200 μl, 1225 μl, 1250 μl, 1275 μl, 1300 μl, 1325 μl, 1350 μl, 1375 μl, 1400 μl, 1425 μl, 1450 μl, 1475 μl, or 1500 μl per side. In some embodiments, the volume of the composition to be infused to the putamen in a human is 900 μl per side.

In some embodiments, the volume of the pharmaceutical composition administered to the thalamus in a human may be no more than 3000 μL/hemisphere. In some embodiments, the volume of the composition to be infused to a thalamus in a human is no more than 2500 μL per side.

In some embodiments, the volume of the composition to be infused to a thalamus in a human is 1300-2500 μL per side. In some embodiments, the volume of the composition to be infused to a thalamus in a human may be 1300 μL, 1325 μL, 1350 μL, 1375 μL, 1400 μL, 1425 μL, 1450 μL, 1475 μL, 1500 μL, 1525 μL, 1550 μL, 1575 μL, 1600 μL, 1625 μL, 1650 μL, 1675 μL, 1700 μL, 1725 μL, 1750 μL, 1775 μL, 1800 μL, 1825 μL, 1850 μL, 1875 μL, 1900 μL, 1925 μL, 1950 μL, 1975 μL, 2000 μL, 2025 μL, 2050 μL, 2075 μL, 2100 μL, 2125 μL, 2150 μL, 2175 μL, 2200 μL, 2225 μL, 2250 μL, 2275 μL, 2300 μL, 2325 μL, 2350 μL, 2375 μL, 2400 μL, 2425 μL, 2450 μL, 2475 μL, or 2500 μL per side. In some embodiments, the volume of the composition to be infused to the thalamus in a human is 1700 μl per side.

In some embodiments, the dose administered to the putamen in a subject may be about 1×10¹⁰ to 1×10¹⁵ VG per side. In some embodiments, the dose administered to the putamen in a subject may be about 1×10¹⁰, 5×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 1.5×10¹², 2×10¹², 2.5×10¹², 3×10¹², 3.5×10¹², 4×10¹², 4.5×10¹², 5×10¹², 5.5×10¹², 6×10¹², 6.5×10¹², 7×10¹², 7.5×10¹², 8×10¹², 8.5×10¹², 9×10¹², 9.5×10¹², 1×10¹³, 1.5×10¹³, 2×10¹³, 2.5×10¹³, 3×10¹³, 3.5×10¹³, 4×10¹³, 4.5×10¹³, 5×10¹³, 5.5×10¹³, 6×10¹³, 6.5×10¹³, 7×10¹³, 7.5×10¹³, 8×10¹³, 8.5×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, or 1×10¹⁵ VG per side.

In some embodiments, the dose administered to the thalamus in a subject may be about 1×10¹⁰ to 1×10¹⁵ VG per side. In some embodiments, the dose administered to the thalamus in a subject may be about 1×10¹⁰, 5×10¹⁰, 1×10¹¹, 2×10¹¹, 3×10¹¹, 4×10¹¹, 5×10¹¹6×10¹¹, 7×10¹¹, 8×10¹¹, 9×10¹¹, 1×10¹², 1.5×10¹², 2×10¹², 2.5×10¹², 3×10¹², 3.5×10¹², 4×10¹², 4.5×10¹², 5×10¹², 5.5×10¹², 6×10¹², 6.5×10¹², 7×10¹², 7.5×10¹², 8×10¹², 8.5×10¹², 9×10¹², 9.5×10¹², 1×10¹³, 1.5×10¹³, 2×10¹³, 2.5×10¹³, 3×10¹³, 3.5×10¹³, 4×10¹³, 4.5×10¹³, 5×10¹³, 5.5×10¹³, 6×10¹³, 6.5×10¹³, 7×10¹³, 7.5×10¹³, 8×10¹³, 8.5×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴ or 1×10¹⁵ VG per side.

In some embodiments, the total dose administered to the subject via putamen and thalamus infusion is 1×10¹⁰ to 5×10¹⁵ VG. In some embodiments, the total dose administered to the subject via putamen and thalamus infusion may be about 1×10¹⁰, 5×10¹⁰, 1×10¹¹ 2×10¹¹, 3×10¹¹, 4×10¹¹, 5×10¹¹, 6×10¹¹, 7×10¹¹, 8×10¹¹, 9×10¹¹, 1×10¹², 1.5×10¹², 2×10¹², 2.5×10¹², 3×10¹², 3.5×10¹², 4×10¹², 4.5×10¹², 5×10¹², 5.5×10¹², 6×10¹², 6.5×10¹², 7×10¹², 7.5×10¹², 8×10¹², 8.5×10¹², 9×10¹², 9.5×10¹², 1×10¹³, 1.5×10¹³, 2×10¹³, 2.5×10¹³, 3×10¹³, 3.5×10¹³, 4×10¹³, 4.5×10¹³, 5×10¹³, 5.5×10¹³, 6×10¹³, 6.5×10¹³, 7×10¹³, 7.5×10¹³, 8×10¹³, 8.5×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, or 5×10¹⁵ VG.

In some embodiments, dose administered to the putamen in a non-human primate may be about 9×10¹⁰ to 5.5×10¹² VG per side. In some embodiments, the dose administered to the putamen in a non-human primate may be about 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 1.5×10¹², 2×10¹², 2.5×10¹², 3×10¹², 3.5×10¹², 4×10¹², 4.5×10¹², 5×10¹², or 5.5×10¹² VG per side.

In some embodiments, the dose administered to the thalamus in a non-human primate may be about 1.5×10¹¹ to 8.5×10¹² VG per side. In some embodiments, the dose administered to the thalamus in a non-human primate may be about 1.5×10¹¹, 1.8×10¹¹, 2×10¹¹, 3×10¹¹, 4×10¹¹, 5×10¹¹, 6×10¹¹, 7×10¹¹, 8×10¹¹, 9×10¹¹, 1×10¹², 1.5×10¹², 2×10¹², 2.5×10¹², 3×10¹², 3.5×10¹², 4×10¹², 4.5×10¹², 5×10¹², 5.5×10¹², 6×10¹², 6.5×10¹², 7×10¹², 7.5×10¹², 8×10¹², or 8.5×10¹² VG per side.

In some embodiments, the total dose administered to the non-human primate via putamen and thalamus infusion is 5×10¹¹ to 3×10¹³ VG. In some embodiments, the total dose administered to the non-human primate via putamen and thalamus infusion may be about 5×10¹¹, 6×10¹¹, 7×10¹¹, 8×10¹¹, 9×10¹¹, 1×10¹², 1.5×10¹², 2×10¹², 2.5×10¹², 3×10¹², 3.5×10¹², 4×10¹², 4.5×10¹², 5×10¹², 5.5×10¹², 6×10¹², 6.5×10¹², 7×10¹², 7.5×10¹², 8×10¹², 8.5×10¹², 9×10¹², 9.5×10¹², 1×10¹³, 1.5×10¹³, 2×10¹³, 2.5×10⁴, or 3×10¹³ VG.

In some embodiments, the dose administered to the putamen in a human may be about 2.5×10¹¹ to 4.5×10¹³ VG per side. In some embodiments, the dose administered to the putamen in a human may be about 2.5×10¹¹, 3×10¹¹, 4×10¹¹, 5×10¹¹, 6×10¹¹, 7×10¹¹, 8×10¹¹, 9×10¹¹, 1×10¹², 1.5×10¹², 2×10¹², 2.5×10¹², 3×10¹², 3.5×10¹², 4×10¹², 4.5×10¹², 5×10¹², 5.5×10¹², 6×10¹², 6.5×10¹², 7×10¹², 7.5×10¹², 8×10¹², 8.5×10¹², 9×10¹², 9.5×10¹², 1×10¹³, 1.5×10¹³, 2×10¹³, 2.5×10¹³, 3×10¹³, 3.5×10¹³, 4×10¹³, or 4.5×10¹³ VG per side. In some embodiments, the dose administered to the putamen in a human may be between 8×10¹¹ to 4×10¹³ VG per side.

In some embodiments, the dose administered to the thalamus in a human may be about 1×10¹² to 7×10¹³ VG per side. In some embodiments, the dose administered to the thalamus in a human may be about 1×10¹², 1.5×10¹², 2×10¹², 2.5×10¹², 3×10¹², 3.5×10¹², 4×10¹², 4.5×10¹², 5×10¹², 5.5×10¹², 6×10¹², 6.5×10¹², 7×10¹², 7.5×10¹², 8×10¹², 8.5×10¹², 9×10¹², 9.5×10¹², 1×10¹³, 1.5×10¹³, 2×10¹³, 2.5×10¹³, 3×10¹³, 3.5×10¹³, 4×10¹³, 4.5×10¹³, 5×10¹³, 5.5×10¹³, 6×10¹³, 6.5×10¹³, 6.8×10¹³, 7×10¹³ VG per side. In some embodiments, the dose administered to the thalamus is between 3.5×10¹² to 6.8×10¹³ VG per side.

In some embodiments, the total dose administered to the human via putamen and thalamus infusion is 2.5×10¹² to 2.5×10¹⁴ VG. In some embodiments, the total dose administered to the human via putamen and thalamus infusion may be about 2.5×10¹², 3×10¹², 3.5×10¹², 4×10¹², 4.5×10¹², 5×10¹², 5.5×10¹², 6×10¹², 6.5×10¹², 7×10¹², 7.5×10¹², 8×10¹², 8.5×10¹², 8.6×10¹², 9×10¹², 9.5×10¹², 1×10¹³, 1.5×10¹³, 2×10¹³, 2.5×10¹³, 3×10¹³, 3.5×10¹³, 4×10¹³, 4.5×10¹³, 5×10¹³, 5.5×10¹³, 6×10¹³, 6.5×10¹³, 7×10¹³, 7.5×10¹³, 8×10¹³, 8.5×10¹³, 9×10¹³, 1×10¹⁴, 2×10¹⁴, 2.1×10¹⁴, 2.2×10¹⁴, 2.3×10¹⁴, 2.4×10¹⁴, or 2.5×10¹⁴ VG. In some embodiments, the total dose administered to the subject is between 8.6×10² to 2×10¹⁴ VG.

In some embodiments, dose volumes may be deposited into infusion site using ascending infusion rates. As a non-limiting example, dose volumes may be deposited into infusion site in 3 different stages (e.g., at dose rates of 1, 3, 5 μL/min) with appropriate durations to complete the total dose volume.

### Exemplary Formulations

In some embodiments, the formulations may include sodium phosphate, potassium phosphate, sodium chloride, potassium chloride, and optionally a surfactant such as Poloxamer 188 (e.g., Pluronic® F-68). As a non-limiting example, the formulation may include 10 mM sodium phosphate, 2 mM potassium phosphate, 192 mM sodium chloride, 2.7 mM potassium chloride, and 0.001% (w/v) Poloxamer 188. The formulations may be used to formulate an AAV particle at a concentration of about 2.7×10¹² VG/mL.

In some embodiments, the formulations may include Phosphate Buffered Saline, sucrose and optionally a surfactant such as Poloxamer 188. As a non-limiting example, the formulation may include Phosphate Buffered Saline, 5% sucrose and 0.001% (w/v) Poloxamer 188. The formulations may be used to formulate an AAV particle at a concentration of about 2.2×10¹² VG/mL.

In some embodiments, the formulations may include sodium phosphate, potassium phosphate, sodium chloride, sucrose and optionally a surfactant such as Poloxamer 188. As a non-limiting example, the formulation may include 2.7 mM sodium phosphate, 1.54 mM potassium phosphate, 155 mM sodium chloride, and 5% (w/v) sucrose at pH 7.2 and with an osmolarity of 450 mOsm/kg.

In some embodiments, the formulations may include sodium phosphate, potassium phosphate, sodium chloride, sucrose, and optionally a surfactant such as Poloxamer 188. As a non-limiting example, the formulation may include 10 mM sodium phosphate, 1.5 mM potassium phosphate, 95 mM sodium chloride, 7% (w/v) sucrose, and 0.001% (w/v) Poloxamer 188, pH 7.4±0.2 at 5° C. The formulations may be used to formulate an AAV particle at a concentration of about 2.7×10¹³ VG/mL.

In some embodiments, the formulation may include Tris Base, hydrochloric acid, potassium chloride, sodium chloride, sucrose, and optionally a surfactant such as Poloxamer 188. As a non-limiting example, the formulation may include 10 mM Tris Base, 6.3 mM HCl, 1.5 mM Potassium Chloride, 100 mM Sodium Chloride, 7% (w/v) Sucrose, and 0.001% (w/v) Poloxamer 188, pH 8.0±0.2 at 5° C. As another non-limiting example, the formulation may include 10 mM Tris Base, 9 mM HCl, 1.5 mM potassium chloride, 100 mM sodium chloride, 7% (w/v) sucrose, and 0.001% (w/v) Poloxamer 188, pH 7.5±0.2 at 5° C. The formulations may be used to formulate an AAV particle at a concentration of about 2.7×10¹³ VG/mL.

### Methods of Treatment of Huntington's Disease

The present disclosure provides AAV particles comprising modulatory polynucleotides encoding siRNA molecules targeting the HTT gene, and methods for their design and manufacture. While not wishing to be bound by a single theory of operability, the AAV particles described herein provide modulatory polynucleotides, including siRNAs, that interfere with HTT expression, including HTT mutant and/or wild-type HT gene expression. Particularly, the present disclosure employs viral genomes such as adeno-associated viral (AAV) viral genomes comprising modulatory polynucleotide sequences encoding the siRNA molecules of the present disclosure. The AAV vectors comprising the modulatory polynucleotides encoding the siRNA molecules of the present disclosure may increase the delivery of active agents into neurons of interest such as medium spiny neurons of the striatum and cortical neurons. The siRNA duplexes or encoded dsRNA targeting the HTT gene may be able to inhibit HTT gene expression (e.g., mRNA level) significantly inside cells; therefore, reducing HIT expression-induced stress inside the cells such as aggregation of protein and formation of inclusions, increased free radicals, mitochondrial dysfunction, and RNA metabolism.

Provided in the present disclosure are methods for introducing the AAV particles comprising a modulatory polynucleotide sequence encoding the siRNA molecules of the present disclosure into cells, the method comprising introducing into said cells any of the AAV particles in an amount sufficient for degradation of target HTT mRNA to occur, thereby activating target-specific RNAi in the cells. In some aspects, the cells may be stem cells, neurons such as medium spiny or cortical neurons, muscle cells and glial cells such as astrocytes.

In some embodiments, the present disclosure provides methods for treating or ameliorating Huntington's Disease (HD) by administering to a subject in need thereof a therapeutically effective amount of a plasmid or AAV vector described herein.

In some embodiments, the AAV particles comprising modulatory polynucleotides encoding the siRNA molecules of the present disclosure may be used to treat and/or ameliorate for HD.

In some embodiments, the AAV particles comprising modulatory polynucleotides encoding the siRNA molecules of the present disclosure may be used to reduce the cognitive and/or motor decline of a subject with HD, where the amount of decline is determined by a standard evaluation system such as, but not limited to, Unified Huntington's Disease Ratings Scale (UHDRS) and sub-scores, and cognitive testing.

In some embodiments, the AAV particles comprising modulatory polynucleotides encoding the siRNA molecules of the present disclosure may be used to reduce the decline of functional capacity and activities of daily living as measured by a standard evaluation system such as, but not limited to, the total functional capacity (TFC) scale.

In some embodiments, the present disclosure provides methods for treating, or ameliorating Huntington's Disease associated with HTT gene and/or HTT protein in a subject in need of treatment, the method comprising administering to the subject a pharmaceutically effective amount of AAV particles comprising modulatory polynucleotides encoding at least one siRNA duplex targeting the HTT gene, inhibiting HTT gene expression and protein production, and ameliorating symptoms of HD in the subject.

In some embodiments, the AAV vectors of the present disclosure may be used as a method of treating Huntington's disease in a subject in need of treatment. Any method known in the art for defining a subject in need of treatment may be used to identify said subject(s). A subject may have a clinical diagnosis of Huntington's disease, or may be pre-symptomatic. Any known method for diagnosing HD may be utilized, including, but not limited to, cognitive assessments and/or neurological or neuropsychiatric examinations, motor tests, sensory tests, psychiatric evaluations, brain imaging, family history and/or genetic testing.

In some embodiments. HD subject selection is determined with the use of the Prognostic Index for Huntington's Disease, or a derivative thereof (Long J D et al., Movement Disorders, 2017, 32(2), 256-263, the contents of which are herein incorporated by reference in their entirety). This prognostic index uses four components to predict probability of motor diagnosis, (1) total motor score (TMS) from the Unified Huntington's Disease Rating Scale (UHDRS), (2) Symbol Digit Modality Test (SDMT), (3) base-line age, and (4) cytosine-adenine-guanine (CAG) expansion.

In some embodiments, the prognostic index for Huntington's Disease is calculated with the following formula: PI_{HD}=51×TMS+(−34)×SDMT+7× Age× (CAG-34), wherein larger values for PI_{HD} indicate greater risk of diagnosis or onset of symptoms.

In another embodiment, the prognostic index for Huntington's Disease is calculated with the following normalized formula that gives standard deviation units to be interpreted in the context of 50% 10-year survival: PIN_{HD}=(PI_{HD}-883)/1044, wherein PIN_{HD}<0 indicates greater than 50% 10-year survival, and PIN_{HD}>0 suggests less than 50% 10-year survival.

In some embodiments, the prognostic index may be used to identify subjects whom will develop symptoms of HD within several years, but that do not yet have clinically diagnosable symptoms. Further, these asymptomatic patients may be selected for and receive treatment using the AAV vectors and compositions of the present disclosure during the asymptomatic period.

In some embodiments, the AAV particles may be administered to a subject who has undergone biomarker assessment. Potential biomarkers in blood for premanifest and early progression of HD include, but are not limited to, 8-OhdG oxidative stress marker, metabolic markers (e.g., creatine kinase, branched-chain amino acids), cholesterol metabolites (e.g., 24-OH cholesterol), immune and inflammatory proteins (e.g., clusterin, complement components, interleukins 6 and 8), gene expression changes (e.g., transcriptomic markers), endocrine markers (e.g., cortisol, ghrelin and leptin), BDNF, adenosine 2A receptors. Potential biomarkers for brain imaging for premanifest and early progression of HD include, but are not limited to, striatal volume, subcortical white-matter volume, cortical thickness, whole brain and ventricular volumes, functional imaging (e.g., functional MRI), PET (e.g., with fluorodeoxyglucose), and magnetic resonance spectroscopy (e.g., lactate). Apart from measurement of huntingtin, among other potential biomarkers is neurofilament light chain, which is a potential marker of neurodegeneration and may be assessed in biofluids such as cerebrospinal fluid or using neuroimaging approaches. Potential biomarkers for quantitative clinical tools for premanifest and early progression of HD include, but are not limited to, quantitative motor assessments, motor physiological assessments (e.g., transcranial magnetic stimulation), and quantitative eye movement measurements. Non-limiting examples of quantitative clinical biomarker assessments include tongue force variability, metronome-guided tapping, grip force, oculomotor assessments and cognitive tests. Non-limiting examples of multicenter observational studies include PREDICT-HD and TRACK-HD. A subject may have symptoms of HD, diagnosed with HD or may be asymptomatic for HD.

In some embodiments, the AAV particles may be administered to a subject who has undergone biomarker assessment using neuroimaging. A subject may have symptoms of HD, diagnosed with HD or may be asymptomatic for HD.

In some embodiments, the AAV particles may be administered to a subject who is asymptomatic for HD. A subject may be asymptomatic but may have undergone predictive genetic testing or biomarker assessment to determine if they are at risk for HD and/or a subject may have a family member (e.g., mother, father, brother, sister, aunt, uncle, grandparent) who has been diagnosed with HD.

In some embodiments, the AAV particles may be administered to a subject who is in the early stages of HD. In the early stage a subject has subtle changes in coordination, some involuntary movements (chorea), changes in mood such as irritability and depression, problem solving difficulties, reduction in the ability of a person to function in their normal day to day life.

In some embodiments, the AAV particles may be administered to a subject who is in the middle stages of HD. In the middle stage a subject has an increase in the movement disorder, diminished speech, difficulty swallowing, and ordinary activities will become harder to do. At this stage a subject may have occupational and physical therapists to help maintain control of voluntary movements and a subject may have a speech language pathologist.

In some embodiments, the AAV particles may be administered to a subject who is in the late stages of HD. In the late stage, a subject with HD is almost completely or completely dependent on others for care as the subject can no longer walk and is unable to speak. A subject can generally still comprehend language and is aware of family and friends, but choking is a major concern.

In some embodiments, the AAV particles may be used to treat a subject who has the juvenile form of HD which is the onset of HD before the age of 20 years and as early as 2 years.

In some embodiments, the AAV particles may be used to treat a subject with HD who has fully penetrant HD where the HTT gene has 41 or more CAG repeats (e.g., 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 or more than 90 CAG repeats).

In some embodiments, the AAV particles may be used to treat a subject with HD who has incomplete penetrance where the HTT gene has between 36 and 40 CAG repeats (e.g., 36, 37, 38, 39 and 40 CAG repeats).

In some embodiments, the composition comprising the AAV particles comprising modulatory polynucleotides encoding the siRNA molecules of the present disclosure is administered to the central nervous system of the subject. In other embodiments, the composition comprising the AAV particles comprising modulatory polynucleotides encoding the siRNA molecules of the present disclosure is administered to a tissue of a subject (e.g., brain of the subject).

In some embodiments, the AAV particles comprising modulatory polynucleotides encoding the siRNA molecules of the present disclosure may be delivered into specific types of targeted cells, including, but not limited to, neurons including medium spiny or cortical neurons; glial cells including oligodendrocytes, astrocytes and microglia; and/or other cells surrounding neurons such as T cells.

In some embodiments, the AAV particles comprising modulatory polynucleotides encoding the siRNA molecules of the present disclosure may be delivered to neurons in the striatum and/or neurons of the cortex.

In some embodiments, the composition of the present disclosure for treating HD is administered to the subject in need intravenously, intramuscularly, subcutaneously, intraperitoneally, intraparenchymally, subpially, intrathecally and/or intraventricularly, allowing the siRNA molecules or vectors comprising the siRNA molecules to pass through one or both the blood-brain barrier and the blood spinal cord barrier, or directly access the brain and/or spinal cord. In some aspects, the method includes administering (e.g., intraparenchymal administration, subpial administration, intraventricular administration and/or intrathecal administration) directly to the central nervous system (CNS) of a subject (using, e.g., an infusion pump and/or a delivery scaffold) a therapeutically effective amount of a composition comprising AAV particles encoding the nucleic acid sequence for the siRNA molecules of the present disclosure. The vectors may be used to silence or suppress HTT gene expression, and/or reducing one or more symptoms of HD in the subject such that HD is therapeutically treated.

In some embodiments, the siRNA molecules or the AAV vectors comprising such siRNA molecules may be introduced directly into the central nervous system of the subject, for example, by infusion into the white matter of a subject. While not wishing to be bound by theory, distribution via direct white matter infusion may be independent of axonal transport mechanisms which may be impaired in subjects with Huntington's Disease which means white matter infusion may allow for more transport of the AAV vectors.

In some embodiments, the composition comprising the AAV particles comprising modulatory polynucleotides encoding the siRNA molecules of the present disclosure is administered to the central nervous system of the subject via intraparenchymal injection.

In some embodiments, the AAV particle composition comprising modulatory polynucleotides encoding the siRNA molecules of the present disclosure is administered to the central nervous system of the subject via intraparenchymal injection and intrathecal injection.

In some embodiments, the AAV particle composition comprising modulatory polynucleotides encoding the siRNA molecules of the present disclosure is administered to the central nervous system of the subject via intraparenchymal injection and intracerebroventricular injection.

In some embodiments, the composition of the present disclosure for treating HD is administered to the subject in need by intraparenchymal administration.

In some embodiments, the AAV particle composition comprising modulatory polynucleotides encoding the siRNA molecules of the present disclosure may be introduced directly into the central nervous system of the subject, for example, by infusion into the putamen.

In some embodiments, the AAV particle composition comprising modulatory polynucleotides encoding the siRNA molecules of the present disclosure may be introduced directly into the central nervous system of the subject, for example, by infusion into the thalamus of a subject. While not wishing to be bound by theory, the thalamus is an area of the brain which is relatively spared in subjects with Huntington's Disease which means it may allow for more widespread cortical transduction via axonal transport of the AAV vectors.

In some embodiments, the AAV particle composition comprising modulatory polynucleotides encoding the siRNA molecules of the present disclosure may be introduced indirectly into the central nervous system of the subject, for example, by intravenous administration.

In some embodiments, AAV particles described herein are administered via putamen and thalamus infusion. Dual infusion into the putamen and thalamus may be independently bilateral or unilateral. As a non-limiting example, AAV particles may be infused into the putamen and thalamus from both sides of the brain. As another non-limiting example, AAV particles may be infused into the left putamen and left thalamus, or right putamen and right thalamus. As yet another non-limiting example, AAV particles may be infused into the left putamen and right thalamus, or right putamen and left thalamus. Dual infusion may occur consecutively or simultaneously.

### Modulate HTT Expression

In some embodiments, administration of the AAV particles to a subject will reduce the expression of HTT in a subject and the reduction of expression of the HTT will reduce the effects of HD in a subject.

In some embodiments, the encoded dsRNA once expressed and contacts a cell expressing HTT protein, inhibits the expression of HTT protein by at least 10%, at least 20%, at least 25%, at least 30%, at least 35% or at least 40% or more, such as when assayed by a method as described herein.

In some embodiments, administration of the AAV particles comprising a modulatory polynucleotide sequence encoding a siRNA of the disclosure, to a subject may lower HTT (e.g., mutant HTT, wild-type HTT and/or mutant and wild-type HTT) in a subject. In some embodiments, administration of the AAV particles to a subject may lower wild-type HTT in a subject. In yet another embodiment, administration of the AAV particles to a subject may lower both mutant HTT and wild-type HTT in a subject. The mutant and/or wild-type HTT may be lowered by about 20%, 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%. 40-50%, 40-60%, 40-70%, 40-80%, 40-90%, 40-95%, 40-100%, 50-60%, 50-70%, 50-80%. 50-90%, 50-95%, 50-100%, 60-70%, 60-80%, 60-90%, 60-95%, 60-100%, 70-80%, 70-90%. 70-95%, 70-100%, 80-90%, 80-95%, 80-100%, 90-95%, 90-100% or 95-100% in a subject such as, but not limited to, the CNS, a region of the CNS, or a specific cell of the CNS of a subject. The mutant HTT may be lowered by about 20%, 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS of a subject. The wild-type HTT may be lowered by about 20%, 30%, 40%, 50%, 60%. 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS of a subject. The mutant and wild-type HTT may be lowered by about 20%, 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS of a subject. As a non-limiting example, the AAV particles may lower the expression of HTT by at least 50% in the medium spiny neurons. As a non-limiting example, the vectors, e.g., AAV vectors may lower the expression of HTT by at least 40% in the medium spiny neurons. As a non-limiting example, the AAV particles may lower the expression of HTT by at least 40% in the medium spiny neurons of the putamen. As a non-limiting example, AAV particles may lower the expression of HTT by at least 30% in the medium spiny neurons of the putamen. As yet another non-limiting example, the AAV particles may lower the expression of HTT in the putamen and cortex by at least 40%. As yet another non-limiting example, the AAV particles may lower the expression of HTT in the putamen and cortex by at least 30%. As yet another non-limiting example, the AAV particles may lower the expression of HTT in the putamen by at least 30%. As yet another non-limiting example, the AAV particles may lower the expression of HTT in the putamen by at least 30% and cortex by at least 15%.

In some embodiments, the AAV particles may be used to reduce the expression of HTT protein by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%. 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. As a non-limiting example, the expression of HTT protein expression may be reduced by 50-90%. As a non-limiting example, the expression of HTT protein expression may be reduced by 30-70%.

In some embodiments, the siRNA duplexes or encoded dsRNA may be used to reduce the expression of HTT mRNA by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%. 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 3040%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-85%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%. 80-95%, 80-100%, 90-95%, 90-100% or 95-100%. As a non-limiting example, the expression of HTT mRNA may be reduced by 50-90%. As a non-limiting example, the expression of HIT mRNA expression may be reduced by 30-70%. As a non-limiting example, the expression of HTT mRNA expression may be reduced by 40-70%. As a non-limiting example, the expression of HTT mRNA expression may be reduced by 50-80%. As a non-limiting example, the expression of HTT mRNA expression may be reduced by 50-85%. As a non-limiting example, the expression of HTT mRNA expression may be reduced by 60-90%.

In some embodiments, the AAV particles may be used to decrease HTT protein in a subject. The decrease may independently be 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 1040%, 1045%, 10-50%, 10-55%, 10-60%, 10-65%, 10-70%, 10-75%, 10-80%, 10-85%, 10-90%, 10-95%, 15-25%, 15-30%, 15-35%, 1540%, 1545%, 15-50%, 15-55%, 15-60%, 15-65%, 15-70%, 15-75%, 15-80%, 15-85%, 15-90%, 15-95%, 20-30%, 20-35%, 2040%, 2045%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 3045%, 30-50%, 30-55%, 30-60%, 30-65%, 30-70%, 30-75%, 30-80%, 30-85%, 30-90%, 30-95%, 3545%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%. As a non-limiting example, a subject may have a 50% decrease of HTT protein. As a non-limiting example, a subject may have a decrease of 70% of HTT protein and a decrease of 10% of wild type HTT protein. As a non-limiting example, the decrease of HTT in the medium spiny neurons of the putamen may be about 40%. As a non-limiting example, the decrease of HTT in neurons of the caudate may be about 30%. As a non-limiting example, the decrease of HTT in neurons of the thalamus may be about 40%. As a non-limiting example, the decrease of HTT in the cortex may be about 20%. As a non-limiting example, the decrease of HTT in pyramidal neurons of the primary motor and somatosensory cortices may be about 30%. As a non-limiting example, the decrease of HTT in the putamen and cortex may be about 40%. As a non-limiting example, the decrease of HTT in the putamen, caudate and cortex may be about 40%. As a non-limiting example, the decrease of HTT in the putamen, caudate, cortex and thalamus may be about 40%. As a non-limiting example, the decrease of HTT in the medium spiny neurons of the putamen may be between 40%-70%. As a non-limiting example, the decrease of HTT in neurons of the caudate may be between 30%-70%. As a non-limiting example, the decrease of HTT in the putamen and cortex may be between 40%-70%. As a non-limiting example, the decrease of HTT in the putamen, caudate and cortex may be between 40%-70%. As a non-limiting example, the decrease of HTT in the putamen, caudate, cortex and thalamus may be between 40%-80%.

In some embodiments, the AAV particles may be used to decrease wild type HIT protein in a subject. The decrease may independently be 5%, 10%, 15%, 20%, 25%, 30%. 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%. 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 3545%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%. As a non-limiting example, a subject may have a 50% decrease of wild type HTT protein. As a non-limiting example, the decrease of wild type HTT in the medium spiny neurons of the putamen may be about 40%. As a non-limiting example, the decrease of wild type HTT in neurons of the caudate may be about 30%. As a non-limiting example, the decrease of wild type HTT in neurons of the thalamus may be about 40%. As a non-limiting example, the decrease of wild type HTT in the cortex may be about 20%. As a non-limiting example, the decrease of wild type HTT in pyramidal neurons of the primary motor and somatosensory cortices may be about 30%. As a non-limiting example, the decrease of wild type HTT in the putamen and cortex may be about 40%. As a non-limiting example, the decrease of wild type HIT in the putamen, caudate and cortex may be about 40%. As a non-limiting example, the decrease of wild type HTT in the putamen, caudate, cortex and thalamus may be about 40%. As a non-limiting example, the decrease of wild type HTT in the medium spiny neurons of the putamen may be between 40%-70%. As a non-limiting example, the decrease of wild type HTT in neurons of the caudate may be between 30%-70%. As a non-limiting example, the decrease of wild type HTT in the putamen and cortex may be between 40%-70%. As a non-limiting example, the decrease of wild type HTT in the putamen, caudate and cortex may be between 40%-70%. As a non-limiting example, the decrease of wild type HTT in the putamen, caudate, cortex and thalamus may be between 40%-80%.

In some embodiments, the AAV particles may be used to decrease mutant HIT protein in a subject. The decrease may independently be 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%. 5-70%, 5-75%, 5-80%, 5-85%, 5-90%, 5-95%, 10-20%, 10-25%, 10-30%, 10-35%, 10-40%, 1045%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 3045%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%. As a non-limiting example, a subject may have a 50% decease of mutant HTT protein. As a non-limiting example, the decrease of mutant HTT in the medium spiny neurons of the putamen may be about 40%. As a non-limiting example, the decrease of mutant HTT in neurons of the caudate may be about 30%. As a non-limiting example, the decrease of mutant HTT in neurons of the thalamus may be about 40%. As a non-limiting example, the decrease of mutant HTT in the cortex may be about 20%. As a non-limiting example, the decrease of mutant HTT in pyramidal neurons of the primary motor and somatosensory cortices may be about 30%. As a non-limiting example, the decrease of mutant HTT in the putamen and cortex may be about 40%. As a non-limiting example, the decrease of mutant HTT in the putamen, caudate and cortex may be about 40%. As a non-limiting example, the decrease of mutant HTT in the putamen, caudate, cortex and thalamus may be about 40%. As a non-limiting example, the decrease of mutant HTT in the medium spiny neurons of the putamen may be between 40%-70%. As a non-limiting example, the decrease of mutant HTT in neurons of the caudate may be between 30%-70%. As a non-limiting example, the decrease of mutant HTT in the putamen and cortex may be between 40%-70%. As a non-limiting example, the decrease of mutant HTT in the putamen, caudate and cortex may be between 40%-70%. As a non-limiting example, the decrease of mutant HTT in the putamen, caudate, cortex and thalamus may be between 40%-80%.

In some embodiments, the present disclosure provides methods for inhibiting/silencing HTT gene expression in a cell. Accordingly, the siRNA duplexes or encoded dsRNA can be used to substantially inhibit HTT gene expression in a cell, in particular in a neuron. In some aspects, the inhibition of HTT gene expression refers to an inhibition by at least about 20%, such as by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 3040%, 30-50%, 30-60%, 30-70%, 30-80%, 30-90%, 30-95%, 30-100%. 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%. 80-100%, 90-95%, 90-100% or 95-100%. Accordingly, the protein product of the targeted gene may be inhibited by at least about 20%, preferably by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 3040%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%. 70-90%, 70-95%, 70-100%, 80-90%, 80-95%, 80-100%, 90-95%, 90-100% or 95-100%.

In some embodiments, the present disclosure provides methods for inhibiting/silencing HTT gene expression in a cell, in particular in a medium spiny neuron. In some aspects, the inhibition of HTT gene expression refers to an inhibition by at least about 20%, such as by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%. 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. Accordingly, the protein product of the targeted gene may be inhibited by at least about 20%, preferably by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 2040%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%. 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%.

In some embodiments, the present disclosure provides methods for inhibiting/silencing HTT gene expression in a cell, in particular in an astrocyte. In some aspects, the inhibition of HTT gene expression refers to an inhibition by at least about 20%, such as by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 3040%, 30-50%, 30-60%, 30-70%, 30-80%, 30-90%, 30-95%, 30-100%, 40-50%, 40-60%, 40-70%, 40-80%. 40-90%, 40-95%, 40-100%, 50-60%, 50-70%, 50-80%, 50-90%, 50-95%, 50-100%, 55-60%. 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. Accordingly, the protein product of the targeted gene may be inhibited by at least about 20%, preferably by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%. 55%, 56%, 57%, 58%, 59%, 60%, 61%, 62%, 63%, 64%, 65%, 66%, 67%, 68%, 69%, 70%. 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%.

In some embodiments, the siRNA duplexes or encoded dsRNA may be used to reduce the expression of HIT protein and/or mRNA in at least one region of the CNS such as, but not limited to the midbrain. The expression of HTT protein and/or mRNA is reduced by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in at least one region of the CNS. As a non-limiting example, the expression of HTT protein and mRNA in the striatum and/or cortex is reduced by 50-90%. As a non-limiting example, the expression of HTT protein and mRNA in the striatum is reduced by 40-50%. As a non-limiting example, the expression of HTT protein and mRNA in the cortex is reduced by 40-50%. As a non-limiting example, the expression of HTT protein and mRNA in the cortex is reduced by 30-70%. As a non-limiting example, the expression of HTT protein and mRNA in the cortex is reduced by at least 30%. As a non-limiting example, the expression of HTT protein and mRNA in the striatum and/or cortex is reduced by 40-70%. As a non-limiting example, the expression of HTT protein and mRNA in the striatum and/or cortex is reduced by 40-50%. As a non-limiting example, the expression of HTT protein and mRNA in the striatum and/or cortex is reduced by 50-70%. As a non-limiting example, the expression of HTT protein and mRNA in the striatum and/or cortex is reduced by 50-60%. As a non-limiting example, the expression of HTT protein and mRNA in the striatum and/or cortex is reduced by 50%. As a non-limiting example, the expression of HTT protein and mRNA in the striatum and/or cortex is reduced by 51%. As a non-limiting example, the expression of HTT protein and mRNA in the striatum and/or cortex is reduced by 52%. As a non-limiting example, the expression of HTT protein and mRNA in the striatum and/or cortex is reduced by 53%. As a non-limiting example, the expression of HTT protein and mRNA in the striatum and/or cortex is reduced by 54%. As a non-limiting example, the expression of HTT protein and mRNA in the striatum and/or cortex is reduced by 55%. As a non-limiting example, the expression of HTT protein and mRNA in the striatum and/or cortex is reduced by 56%. As a non-limiting example, the expression of HTT protein and mRNA in the striatum and/or cortex is reduced by 57%. As a non-limiting example, the expression of HTT protein and mRNA in the striatum and/or cortex is reduced by 58%. As a non-limiting example, the expression of HTT protein and mRNA in the striatum and/or cortex is reduced by 59%. As a non-limiting example, the expression of HTT protein and mRNA in the striatum and/or cortex is reduced by 60%. As a non-limiting example, the expression of HTT protein and mRNA in the striatum, thalamus, and/or cortex is reduced by at least 20%. As a non-limiting example, the expression of HTT protein and mRNA in the striatum, thalamus, and/or cortex is reduced by 30%. As a non-limiting example, the expression of HTT protein and mRNA in the striatum, thalamus, and/or cortex is reduced by 30-70%. As a non-limiting example, the expression of HTT protein and mRNA in the striatum, thalamus, and/or cortex is reduced by 40-80%. As a non-limiting example, the expression of HTT protein and mRNA in the striatum, thalamus, and/or cortex is reduced by 40-70%. As a non-limiting example, the expression of HTT protein and mRNA in the striatum, thalamus, and/or cortex is reduced by 40-60%. As a non-limiting example, the expression of HTT protein and mRNA in the striatum, thalamus, and/or cortex is reduced by 50-80%. As a non-limiting example, the expression of HTT protein and mRNA in the striatum, thalamus, and/or cortex is reduced by 50-70%.

In some embodiments, the present disclosure provides methods for inhibiting/silencing HTT gene expression in a cell, in particular in a pyramidal neuron of the primary motor cortex or primary somatosensory cortex. In some aspects, the inhibition of HTT gene expression refers to an inhibition by at least about 20%, such as by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%. 55-95%, 55-100%, 60-70%, 60-80%, 60-90%, 60-95%, 60-100%, 70-80%, 70-90%, 70-95%. 70-100%, 80-90%, 80-95%, 80-100%, 90-95%, 90-100% or 95-100%. Accordingly, the protein product of the targeted gene may be inhibited by at least about 20%, preferably by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%. 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%.

In some embodiments, the siRNA duplexes or encoded dsRNA may be used to reduce the expression of HTT protein and/or mRNA in at least one region of the CNS such as, but not limited to the forebrain. The expression of HTT protein and/or mRNA is reduced by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%. 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in at least one region of the CNS. As a non-limiting example, the expression of HTT protein and/or mRNA in the putamen is reduced by 50-90%. As a non-limiting example, the expression of HTT protein and/or mRNA in the striatum is reduced by 40-50%. As a non-limiting example, the expression of HTT protein and/or mRNA in the cortex is reduced by 40-50%. As a non-limiting example, the expression of HTT protein and/or mRNA in the cortex is reduced by 30-70%.

As a non-limiting example, the expression of HTT protein and/or mRNA in the striatum and/or cortex is reduced by 40-70%. As a non-limiting example, the expression of HTT protein and/or mRNA in the striatum and/or cortex is reduced by 40-50%. As a non-limiting example, the expression of HTT protein and/or mRNA in the striatum and/or cortex is reduced by 50-70%. As a non-limiting example, the expression of HTT protein and/or mRNA in the striatum and/or cortex is reduced by 50-60%. As a non-limiting example, the expression of HTT protein and/or mRNA in the striatum and/or cortex is reduced by 50%. As a non-limiting example, the expression of HTT protein and/or mRNA in the striatum and/or cortex is reduced by 51%. As a non-limiting example, the expression of HTT protein and/or mRNA in the striatum and/or cortex is reduced by 52%. As a non-limiting example, the expression of HTT protein and/or mRNA in the striatum and/or cortex is reduced by 53%. As a non-limiting example, the expression of HTT protein and/or mRNA in the striatum and/or cortex is reduced by 54%. As a non-limiting example, the expression of HTT protein and/or mRNA in the striatum and/or cortex is reduced by 55%. As a non-limiting example, the expression of HTT protein and/or mRNA in the striatum and/or cortex is reduced by 56%. As a non-limiting example, the expression of HTT protein and/or mRNA in the striatum and/or cortex is reduced by 57%. As a non-limiting example, the expression of HTT protein and/or mRNA in the striatum and/or cortex is reduced by 58%. As a non-limiting example, the expression of HTT protein and/or mRNA in the striatum and/or cortex is reduced by 59%. As a non-limiting example, the expression of HTT protein and/or mRNA in the striatum and/or cortex is reduced by 60%. As a non-limiting example, the expression of HTT protein and/or mRNA in the striatum and/or cortex is reduced by 61%. As a non-limiting example, the expression of HTT protein and/or mRNA in the striatum and/or cortex is reduced by 62%. As a non-limiting example, the expression of HTT protein and/or mRNA in the striatum and/or cortex is reduced by 63%. As a non-limiting example, the expression of HTT protein and/or mRNA in the striatum and/or cortex is reduced by 64%. As a non-limiting example, the expression of HTT protein and/or mRNA in the striatum and/or cortex is reduced by 65%. As a non-limiting example, the expression of HTT protein and/or mRNA in the striatum and/or cortex is reduced by 66%. As a non-limiting example, the expression of HTT protein and/or mRNA in the striatum and/or cortex is reduced by 67%. As a non-limiting example, the expression of HIT protein and/or mRNA in the striatum and/or cortex is reduced by 68%. As a non-limiting example, the expression of HTT protein and/or mRNA in the striatum and/or cortex is reduced by 69%. As a non-limiting example, the expression of HTT protein and/or mRNA in the striatum and/or cortex is reduced by 70%.

In some embodiments, the siRNA duplexes or encoded dsRNA may be used to reduce the expression of HTT protein and/or mRNA in the striatum. The expression of HTT protein and/or mRNA is reduced by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%. 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%. 70-100%, 80-90%, 80-95%, 80-100%, 90-95%, 90-100% or 95-100%. As a non-limiting example, the expression of HTT protein and/or mRNA in the striatum is reduced by 40-50%. As a non-limiting example, the expression of HTT protein and/or mRNA in the striatum is reduced by 30-70%. As a non-limiting example, the expression of HTT protein and/or mRNA in the striatum is reduced by at least 30%. As a non-limiting example, the expression of HTT protein and/or mRNA in the striatum is reduced by 40-70%. As a non-limiting example, the expression of HTT protein and/or mRNA in the striatum is reduced by 40-50%. As a non-limiting example, the expression of HTT protein and/or mRNA in the striatum is reduced by 50-70%. As a non-limiting example, the expression of HTT protein and/or mRNA in the striatum is reduced by 50-60%. As a non-limiting example, the expression of HTT protein and/or mRNA in the striatum is reduced by 50%. As a non-limiting example, the expression of HTT protein and/or mRNA in the striatum is reduced by 51%. As a non-limiting example, the expression of HTT protein and/or mRNA in the striatum is reduced by 52%. As a non-limiting example, the expression of HTT protein and/or mRNA in the striatum is reduced by 53%. As a non-limiting example, the expression of HTT protein and/or mRNA in the striatum is reduced by 54%. As a non-limiting example, the expression of HTT protein and/or mRNA in the striatum is reduced by 55%. As a non-limiting example, the expression of HTT protein and/or mRNA in the striatum is reduced by 56%. As a non-limiting example, the expression of HTT protein and/or mRNA in the striatum is reduced by 57%. As a non-limiting example, the expression of HTT protein and/or mRNA in the striatum is reduced by 58%. As a non-limiting example, the expression of HTT protein and/or mRNA in the striatum is reduced by 59%. As a non-limiting example, the expression of HTT protein and/or mRNA in the striatum is reduced by 60%. As a non-limiting example, the expression of HTT protein and/or mRNA in the striatum is reduced by 61%. As anon-limiting example, the expression of HTT protein and/or mRNA in the striatum is reduced by 62%. As a non-limiting example, the expression of HTT protein and/or mRNA in the striatum is reduced by 63%. As a non-limiting example, the expression of HTT protein and/or mRNA in the striatum is reduced by 64%. As a non-limiting example, the expression of HTT protein and/or mRNA in the striatum is reduced by 65%. As a non-limiting example, the expression of HTT protein and/or mRNA in the striatum is reduced by 66%. As a non-limiting example, the expression of HTT protein and/or mRNA in the striatum is reduced by 67%. As a non-limiting example, the expression of HTT protein and/or mRNA in the striatum is reduced by 68%. As a non-limiting example, the expression of HTT protein and/or mRNA in the striatum is reduced by 69%. As a non-limiting example, the expression of HTT protein and/or mRNA in the striatum is reduced by 70%.

In some embodiments, the AAV particles comprising modulatory polynucleotides encoding the siRNA molecules of the present disclosure may be used to suppress HTT protein in neurons and/or astrocytes of the striatum and/or the cortex. As a non-limiting example, the suppression of HTT protein is in medium spiny neurons of the striatum and/or neurons of the cortex. As a non-limiting example, the suppression of HTT protein is in medium spiny neurons of the striatum and/or pyramidal neurons of the primary motor cortex and primary somatosensory cortex.

In some embodiments, the AAV particles comprising modulatory polynucleotides encoding the siRNA molecules of the present disclosure may be used to suppress HTT protein in neurons and/or astrocytes of the striatum and/or the cortex and reduce associated neuronal toxicity. The suppression of HTT protein in the neurons and/or astrocytes of the striatum and/or the cortex may be, independently, suppressed by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 1540%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%. The reduction of associated neuronal toxicity may be 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 2540%, 2545%, 25-50%, 25-55%, 25-60%, 25-65%, 25-70%, 25-75%, 25-80%, 25-85%, 25-90%, 25-95%, 3040%, 3045%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In some embodiments, the siRNA duplexes or encoded dsRNA may be used to reduce the expression of HTT protein and/or mRNA in the cortex. The expression of HTT protein and/or mRNA is reduced by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 3040%, 30-50%, 30-60%, 30-70%, 30-80%, 30-90%, 30-95%, 30-100%, 40-50%. 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. As a non-limiting example, the expression of HTT protein and/or mRNA in the cortex is reduced by 40-50%. As a non-limiting example, the expression of HTT protein and/or mRNA in the cortex is reduced by 30-70%. As a non-limiting example, the expression of HTT protein and/or mRNA in the cortex is reduced by at least 30%. As a non-limiting example, the expression of HTT protein and/or mRNA in the cortex is reduced by 40-70%. As a non-limiting example, the expression of HTT protein and/or mRNA in the cortex is reduced by 40-50%. As a non-limiting example, the expression of HTT protein and/or mRNA in the cortex is reduced by 50-70%. As a non-limiting example, the expression of HTT protein and/or mRNA in the cortex is reduced by 50-60%. As a non-limiting example, the expression of HTT protein and/or mRNA in the cortex is reduced by 50%. As a non-limiting example, the expression of HTT protein and/or mRNA in the cortex is reduced by 51%. As a non-limiting example, the expression of HIT protein and/or mRNA in the cortex is reduced by 52%. As a non-limiting example, the expression of HTT protein and/or mRNA in the cortex is reduced by 53%. As a non-limiting example, the expression of HTT protein and/or mRNA in the cortex is reduced by 54%. As a non-limiting example, the expression of HTT protein and/or mRNA in the cortex is reduced by 55%. As a non-limiting example, the expression of HTT protein and/or mRNA in the cortex is reduced by 56%. As a non-limiting example, the expression of HTT protein and/or mRNA in the cortex is reduced by 57%. As a non-limiting example, the expression of HTT protein and/or mRNA in the cortex is reduced by 58%. As a non-limiting example, the expression of HTT protein and/or mRNA in the cortex is reduced by 59%. As a non-limiting example, the expression of HTT protein and/or mRNA in the cortex is reduced by 60%.

In some embodiments, the siRNA duplexes or encoded dsRNA may be used to reduce the expression of HTT protein and/or mRNA in the motor cortex. The expression of HIT protein and/or mRNA is reduced by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. As a non-limiting example, the expression of HTT protein and/or mRNA in the motor cortex is reduced by 20-30%. As a non-limiting example, the expression of HTT protein and/or mRNA in the motor cortex is reduced by 40-50%. As a non-limiting example, the expression of HTT protein and/or mRNA in the motor cortex is reduced by 30-70%. As a non-limiting example, the expression of HTT protein and/or mRNA in the motor cortex is reduced by at least 30%. As a non-limiting example, the expression of HTT protein and/or mRNA in the motor cortex is reduced by 40-70%. As a non-limiting example, the expression of HTT protein and/or mRNA in the motor cortex is reduced by 40-50%. As a non-limiting example, the expression of HTT protein and/or mRNA in the motor cortex is reduced by 50-70%. As a non-limiting example, the expression of HTT protein and/or mRNA in the motor cortex is reduced by 50-60%. As a non-limiting example, the expression of HTT protein and/or mRNA in the motor cortex is reduced by 50%. As a non-limiting example, the expression of HTT protein and/or mRNA in the motor cortex is reduced by 51%. As a non-limiting example, the expression of HTT protein and/or mRNA in the motor cortex is reduced by 52%. As a non-limiting example, the expression of HTT protein and/or mRNA in the motor cortex is reduced by 53%. As a non-limiting example, the expression of HTT protein and/or mRNA in the motor cortex is reduced by 54%. As a non-limiting example, the expression of HTT protein and/or mRNA in the motor cortex is reduced by 55%. As a non-limiting example, the expression of HTT protein and/or mRNA in the motor cortex is reduced by 56%. As a non-limiting example, the expression of HTT protein and/or mRNA in the motor cortex is reduced by 57%. As a non-limiting example, the expression of HTT protein and/or mRNA in the motor cortex is reduced by 58%. As a non-limiting example, the expression of HTT protein and/or mRNA in the motor cortex is reduced by 59%. As a non-limiting example, the expression of HTT protein and/or mRNA in the motor cortex is reduced by 60%.

In some embodiments, the siRNA duplexes or encoded dsRNA may be used to reduce the expression of HIT protein and/or mRNA in the somatosensory cortex. The expression of HTT protein and/or mRNA is reduced by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%. 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-10%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. As a non-limiting example, the expression of HTT protein and/or mRNA in the somatosensory cortex is reduced by 20-30%. As a non-limiting example, the expression of HTT protein and/or mRNA in the somatosensory cortex is reduced by 40-50%. As a non-limiting example, the expression of HTT protein and/or mRNA in the somatosensory cortex is reduced by 30-70%. As a non-limiting example, the expression of HTT protein and/or mRNA in the somatosensory cortex is reduced by at least 30%. As a non-limiting example, the expression of HTT protein and/or mRNA in the somatosensory cortex is reduced by 40-70%. As a non-limiting example, the expression of HTT protein and/or mRNA in the somatosensory cortex is reduced by 40-50%. As a non-limiting example, the expression of HTT protein and/or mRNA in the somatosensory cortex is reduced by 50-70%. As a non-limiting example, the expression of HT protein and/or mRNA in the somatosensory cortex is reduced by 50-60%. As a non-limiting example, the expression of HTT protein and/or mRNA in the somatosensory cortex is reduced by 50%. As a non-limiting example, the expression of HTT protein and/or mRNA in the somatosensory cortex is reduced by 51%. As a non-limiting example, the expression of HTT protein and/or mRNA in the somatosensory cortex is reduced by 52%. As a non-limiting example, the expression of HTT protein and/or mRNA in the somatosensory cortex is reduced by 53%. As a non-limiting example, the expression of HIT protein and/or mRNA in the somatosensory cortex is reduced by 54%. As a non-limiting example, the expression of HTT protein and/or mRNA in the somatosensory cortex is reduced by 55%. As a non-limiting example, the expression of HTT protein and/or mRNA in the somatosensory cortex is reduced by 56%. As a non-limiting example, the expression of HTT protein and/or mRNA in the somatosensory cortex is reduced by 57%. As a non-limiting example, the expression of HTT protein and/or mRNA in the somatosensory cortex is reduced by 58%. As a non-limiting example, the expression of HTT protein and/or mRNA in the somatosensory cortex is reduced by 59%. As a non-limiting example, the expression of HTT protein and/or mRNA in the somatosensory cortex is reduced by 60%.

In some embodiments, the siRNA duplexes or encoded dsRNA may be used to reduce the expression of HTT protein and/or mRNA in the temporal cortex. The expression of HTT protein and/or mRNA is reduced by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%. 52%, 53%, 54%, 55%, 56%, 57%, 58%, 59%, 60%, 61%, 62%, 63%, 64%, 65%, 66%, 67%. 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%. 70-95%, 70-100%, 80-90%, 80-95%, 80-100%, 90-95%, 90-100% or 95-100%. As a non-limiting example, the expression of HTT protein and/or mRNA in the temporal cortex is reduced by 40-50%. As a non-limiting example, the expression of HIT protein and/or mRNA in the temporal cortex is reduced by 30-70%. As a non-limiting example, the expression of HTT protein and/or mRNA in the temporal cortex is reduced by at least 30%. As a non-limiting example, the expression of HTT protein and/or mRNA in the temporal cortex is reduced by 40-70%. As a non-limiting example, the expression of HTT protein and/or mRNA in the temporal cortex is reduced by 40-50%. As a non-limiting example, the expression of HTT protein and/or mRNA in the temporal cortex is reduced by 50-70%. As a non-limiting example, the expression of HTT protein and/or mRNA in the temporal cortex is reduced by 50-60%. As a non-limiting example, the expression of HTT protein and/or mRNA in the temporal cortex is reduced by 50%. As a non-limiting example, the expression of HTT protein and/or mRNA in the temporal cortex is reduced by 51%. As a non-limiting example, the expression of HTT protein and/or mRNA in the temporal cortex is reduced by 52%. As a non-limiting example, the expression of HTT protein and/or mRNA in the temporal cortex is reduced by 53%. As a non-limiting example, the expression of HTT protein and/or mRNA in the temporal cortex is reduced by 54%. As a non-limiting example, the expression of HTT protein and/or mRNA in the temporal cortex is reduced by 55%. As a non-limiting example, the expression of HTT protein and/or mRNA in the temporal cortex is reduced by 56%. As a non-limiting example, the expression of HTT protein and/or mRNA in the temporal cortex is reduced by 57%. As a non-limiting example, the expression of HTT protein and/or mRNA in the temporal cortex is reduced by 58%. As a non-limiting example, the expression of HTT protein and/or mRNA in the temporal cortex is reduced by 59%. As a non-limiting example, the expression of HTT protein and/or mRNA in the temporal cortex is reduced by 60%.

In some embodiments, the siRNA duplexes or encoded dsRNA may be used to reduce the expression of HTT protein and/or mRNA in the putamen. The expression of HTT protein and/or mRNA is reduced by at least about 30%, 31%, 320, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 55-60%, 55-70%, 55-80%, 55-90%, 55-95%, 55-100%, 60-70%, 60-80%. 60-90%, 60-95%, 60-100%, 70-80%, 70-90%, 70-95%, 70-100%, 80-90%, 80-95%, 80-100%, 90-95%, 90-100% or 95-100% in the putamen. As a non-limiting example, the expression of HTT protein and/or mRNA in the putamen is reduced by 40-70%. As a non-limiting example, the expression of HTT protein and/or mRNA in the putamen is reduced by 30-40%. As a non-limiting example, the expression of HTT protein and/or mRNA in the putamen is reduced by 40-50%. As a non-limiting example, the expression of HTT protein and/or mRNA in the putamen is reduced by 50-80%. As a non-limiting example, the expression of HTT protein and/or mRNA in the putamen is reduced by 50-70%. As a non-limiting example, the expression of HTT protein and/or mRNA in the putamen is reduced by 50-60%. As a non-limiting example, the expression of HTT protein and/or mRNA in the putamen is reduced by 60-70%. As a non-limiting example, the expression of HTT protein and/or mRNA in the putamen is reduced by 50%. As a non-limiting example, the expression of HTT protein and/or mRNA in the putamen is reduced by 51%. As a non-limiting example, the expression of HTT protein and/or mRNA in the putamen is reduced by 52%. As a non-limiting example, the expression of HTT protein and/or mRNA in the putamen is reduced by 53%. As a non-limiting example, the expression of HTT protein and/or mRNA in the putamen is reduced by 54%. As a non-limiting example, the expression of HTT protein and/or mRNA in the putamen is reduced by 55%. As a non-limiting example, the expression of HTT protein and/or mRNA in the putamen is reduced by 56%. As a non-limiting example, the expression of HTT protein and/or mRNA in the putamen is reduced by 57%. As a non-limiting example, the expression of HTT protein and/or mRNA in the putamen is reduced by 58%. As a non-limiting example, the expression of HTT protein and/or mRNA in the putamen is reduced by 59%. As a non-limiting example, the expression of HTT protein and/or mRNA in the putamen is reduced by 60%. As a non-limiting example, the expression of HTT protein and/or mRNA in the putamen is reduced by 61%. As a non-limiting example, the expression of HT protein and/or mRNA in the putamen is reduced by 62%. As a non-limiting example, the expression of HTT protein and/or mRNA in the putamen is reduced by 63%. As a non-limiting example, the expression of HTT protein and/or mRNA in the putamen is reduced by 64%. As a non-limiting example, the expression of HTT protein and/or mRNA in the putamen is reduced by 65%. As a non-limiting example, the expression of HTT protein and/or mRNA in the putamen is reduced by 66%. As a non-limiting example, the expression of HTT protein and/or mRNA in the putamen is reduced by 67%. As a non-limiting example, the expression of HTT protein and/or mRNA in the putamen is reduced by 68%. As a non-limiting example, the expression of HTT protein and/or mRNA in the putamen is reduced by 69%. As a non-limiting example, the expression of HTT protein and/or mRNA in the putamen is reduced by 70%.

In some embodiments, the siRNA duplexes or encoded dsRNA may be used to reduce the expression of HTT protein and/or mRNA in the caudate. The expression of HTT protein and/or mRNA is reduced by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-85%, 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in the caudate. As a non-limiting example, the expression of HTT protein and/or mRNA in the caudate is reduced by 40-70%. As a non-limiting example, the expression of HTT protein and/or mRNA in the caudate is reduced by 40-50%. As a non-limiting example, the expression of HTT protein and/or mRNA in the caudate is reduced by 50-85%. As a non-limiting example, the expression of HTT protein and/or mRNA in the caudate is reduced by 50-80%. As a non-limiting example, the expression of HTT protein and/or mRNA in the caudate is reduced by 50-70%. As a non-limiting example, the expression of HTT protein and/or mRNA in the caudate is reduced by 50-60%. As a non-limiting example, the expression of HTT protein and/or mRNA in the caudate is reduced by 60-70%. As a non-limiting example, the expression of HTT protein and/or mRNA in the caudate is reduced by 50%. As a non-limiting example, the expression of HTT protein and/or mRNA in the caudate is reduced by 51%. As a non-limiting example, the expression of HTT protein and/or mRNA in the caudate is reduced by 52%. As a non-limiting example, the expression of HTT protein and/or mRNA in the caudate is reduced by 53%. As a non-limiting example, the expression of HTT protein and/or mRNA in the caudate is reduced by 54%. As a non-limiting example, the expression of HTT protein and/or mRNA in the caudate is reduced by 55%. As a non-limiting example, the expression of HTT protein and/or mRNA in the caudate is reduced by 56%. As a non-limiting example, the expression of HTT protein and/or mRNA in the caudate is reduced by 57%. As a non-limiting example, the expression of HT protein and/or mRNA in the caudate is reduced by 58%. As a non-limiting example, the expression of HTT protein and/or mRNA in the caudate is reduced by 59%. As a non-limiting example, the expression of HTT protein and/or mRNA in the caudate is reduced by 60%. As a non-limiting example, the expression of HTT protein and/or mRNA in the caudate is reduced by 61%. As a non-limiting example, the expression of HTT protein and/or mRNA in the caudate is reduced by 62%. As a non-limiting example, the expression of HTT protein and/or mRNA in the caudate is reduced by 63%. As a non-limiting example, the expression of HTT protein and/or mRNA in the caudate is reduced by 64%. As a non-limiting example, the expression of HTT protein and/or mRNA in the caudate is reduced by 65%. As a non-limiting example, the expression of HTT protein and/or mRNA in the caudate is reduced by 66%. As a non-limiting example, the expression of HTT protein and/or mRNA in the caudate is reduced by 67%. As a non-limiting example, the expression of HTT protein and/or mRNA in the caudate is reduced by 68%. As a non-limiting example, the expression of HTT protein and/or mRNA in the caudate is reduced by 69%. As a non-limiting example, the expression of HTT protein and/or mRNA in the caudate is reduced by 70%.

In some embodiments, the siRNA duplexes or encoded dsRNA may be used to reduce the expression of HTT protein and/or mRNA in the thalamus. The expression of HTT protein and/or mRNA is reduced by at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%. 40-60%, 40-70%, 40-80%, 40-90%, 40-95%, 40-100%, 50-60%, 50-70%, 50-80%, 50-85%. 50-90%, 50-95%, 50-100%, 55-60%, 55-70%, 55-80%, 55-90%, 55-95%, 55-100%, 60-70%, 60-80%, 60-90%, 60-95%, 60-100%, 70-80%, 70-90%, 70-95%, 70-10%, 80-90%, 80-95%, 80-100%, 90-95%, 90-100% or 95-100% in the thalamus. As a non-limiting example, the expression of HTT protein and/or mRNA in the thalamus is reduced by at least 30%. As a non-limiting example, the expression of HTT protein and/or mRNA in the thalamus is reduced by 40-70%. As a non-limiting example, the expression of HTT protein and/or mRNA in the thalamus is reduced by 40-80%. As a non-limiting example, the expression of HTT protein and/or mRNA in the thalamus is reduced by 60-90%. As a non-limiting example, the expression of HTT protein and/or mRNA in the thalamus is reduced by 60-80%. As a non-limiting example, the expression of HTT protein and/or mRNA in the thalamus is reduced by 60-70%. As a non-limiting example, the expression of HTT protein and/or mRNA in the thalamus is reduced by 60%. As a non-limiting example, the expression of HTT protein and/or mRNA in the thalamus is reduced by 61%. As a non-limiting example, the expression of HTT protein and/or mRNA in the thalamus is reduced by 62%. As a non-limiting example, the expression of HTT protein and/or mRNA in the thalamus is reduced by 63%. As a non-limiting example, the expression of HTT protein and/or mRNA in the thalamus is reduced by 64%. As a non-limiting example, the expression of HTT protein and/or mRNA in the thalamus is reduced by 65%. As a non-limiting example, the expression of HTT protein and/or mRNA in the thalamus is reduced by 66%. As a non-limiting example, the expression of HTT protein and/or mRNA in the thalamus is reduced by 67%. As a non-limiting example, the expression of HTT protein and/or mRNA in the thalamus is reduced by 68%. As a non-limiting example, the expression of HTT protein and/or mRNA in the thalamus is reduced by 69%. As a non-limiting example, the expression of HTT protein and/or mRNA in the thalamus is reduced by 70%. As a non-limiting example, the expression of HTT protein and/or mRNA in the thalamus is reduced by 71%. As a non-limiting example, the expression of HTT protein and/or mRNA in the thalamus is reduced by 72%. As a non-limiting example, the expression of HTT protein and/or mRNA in the thalamus is reduced by 73%. As a non-limiting example, the expression of HTT protein and/or mRNA in the thalamus is reduced by 74%. As a non-limiting example, the expression of HTT protein and/or mRNA in the thalamus is reduced by 75%. As a non-limiting example, the expression of HTT protein and/or mRNA in the thalamus is reduced by 76%. As a non-limiting example, the expression of HTT protein and/or mRNA in the thalamus is reduced by 77%. As a non-limiting example, the expression of HTT protein and/or mRNA in the thalamus is reduced by 78%. As a non-limiting example, the expression of HTT protein and/or mRNA in the thalamus is reduced by 79%. As a non-limiting example, the expression of HTT protein and/or mRNA in the thalamus is reduced by 80%.

In some embodiments, AAV particles encoding siRNA duplexes, or pharmaceutical compositions thereof, have a half maximal effective concentration (EC₅₀) of about 1-300 VG/cell. The half maximal effective concentration (EC₅₀), as used herein, refers to the concentration of AAV vectors encoding siRNA duplexes that produces 50% reduction in HTT expression in a cell. HTT expression may be HTT mRNA or protein expression. AAV particles encoding siRNA duplexes, or pharmaceutical compositions thereof, may have an EC₅₀ of 1-10, 1-20, 1-30, 1-40, 1-50, 10-20, 10-30, 10-40, 10-50, 10-60, 15-30, 20-30, 20-40, 20-50, 20-60, 20-70, 3040, 30-50, 30-60, 30-70, 30-80, 35-50, 40-50, 40-60, 40-70, 40-80, 40-90, 50-60, 50-70, 50-80, 50-90, 50-100, 60-70, 60-80, 60-90, 60-100, 70-90, 70-100, 70-120, 80-100, 80-120, 80-140, 90-120, 90-150, 90-180, 100-120, 100-150, 100-180, 100-200, 120-160, 120-180, 150-200, 200-250, 200-300, or 250-300 VG/cell. For example, the AAV particles encoding siRNA duplexes, or pharmaceutical compositions thereof, may have an EC₅₀ of about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, 190, 200, 250, or 300 VG/cell. As a non-limiting example, AAV particles encoding siRNA duplexes, or pharmaceutical compositions thereof, may have an EC₅₀ of about 35-50 VG/cell in the putamen. As another non-limiting example, AAV particles encoding siRNA duplexes, or pharmaceutical compositions thereof, may have an EC₅₀ of about 15-30 VG/cell in the caudate.

### Solo and Combination Therapy

In some embodiments, the present composition is administered as a solo therapeutic or combination therapeutics for the treatment of HD.

In some embodiments, the pharmaceutical composition of the present disclosure is used as a solo therapy. In other embodiments, the pharmaceutical composition of the present disclosure is used in combination therapy. The combination therapy may be in combination with one or more neuroprotective agents such as small molecule compounds, growth factors and hormones which have been tested for their neuroprotective effect on neuron degeneration.

The AAV particles encoding siRNA duplexes targeting the HTT gene may be used in combination with one or more other therapeutic agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent.

Therapeutic agents that may be used in combination with the AAV particles encoding the nucleic acid sequence for the siRNA molecules of the present disclosure can be small molecule compounds which are antioxidants, anti-inflammatory agents, anti-apoptosis agents, calcium regulators, antiglutamatergic agents, structural protein inhibitors, compounds involved in muscle function, and compounds involved in metal ion regulation.

Compounds tested for treating HD which may be used in combination with the vectors described herein include, but are not limited to, dopamine-depleting agents (e.g., tetrabenazine for chorea), benzodiazepines (e.g., clonazepam for myoclonus, chorea, dystonia, rigidity, and/or spasticity), anticonvulsants (e.g., sodium valproate and levetiracetam for myoclonus), amino acid precursors of dopamine (e.g., levodopa for rigidity which is particularly associate with juvenile HD or young adult-onset parkinsonian phenotype), skeletal muscle relaxants (e.g., baclofen, tizanidine for rigidity and/or spasticity), inhibitors for acetylcholine release at the neuromuscular junction to cause muscle paralysis (e.g., botulinum toxin for bruxism and/or dystonia), atypical neuroleptics (e.g., olanzapine and quetiapine for psychosis and/or irritability, risperidone, sulpiride and haloperidol for psychosis, chorea and/or irritability, clozapine for treatment-resistant psychosis, aripiprazole for psychosis with prominent negative symptoms), agents to increase ATP/cellular energetics (e.g., creatine), selective serotonin reuptake inhibitors (SSRIs) (e.g., citalopram, fluoxetine, paroxetine, sertraline, mirtazapine, venlafaxine for depression, anxiety, obsessive compulsive behavior and/or irritability), hypnotics (e.g., xopiclone and/or zolpidem for altered sleep-wake cycle), anticonvulsants (e.g., sodium valproate and carbamazepine for mania or hypomania) and mood stabilizers (e.g., lithium for mania or hypomania).

Neurotrophic factors may be used in combination therapy with the AAV particles encoding the nucleic acid sequence for the siRNA molecules of the present disclosure for treating HD. Generally, a neurotrophic factor is defined as a substance that promotes survival, growth, differentiation, proliferation and/or maturation of a neuron, or stimulates increased activity of a neuron. In some embodiments, the present methods further comprise delivery of one or more trophic factors into the subject in need of treatment. Trophic factors may include, but are not limited to, IGF-I, GDNF, BDNF, CTNF, VEGF, Colivelin, Xaliproden, Thyrotrophin-releasing hormone and ADNF, and variants thereof.

In one aspect, the AAV particles comprising modulatory polynucleotides encoding the siRNA duplex targeting the HTT gene may be co-administered with AAV vectors expressing neurotrophic factors such as AAV-IGF-I (See e.g., Vincent et al., Neuromolecular medicine, 2004, 6, 79-85: the content of which is incorporated herein by reference in its entirety) and AAV-GDNF (See e.g., Wang et al., J Neurosci., 2002, 22, 6920-6928; the content of which is incorporated herein by reference in its entirety).

### VI. Definitions

At various places in the present disclosure, substituents or properties of compounds of the present disclosure are disclosed in groups or in ranges. It is specifically intended that the present disclosure include each and every individual or subcombination of the members of such groups and ranges.

Unless stated otherwise, the following terms and phrases have the meanings described below. The definitions are not meant to be limiting in nature and serve to provide a clearer understanding of certain aspects of the present disclosure.

About: As used herein, the term “about” means+/−10% of the recited value.

Adeno-associated virus: The term “adeno-associated virus” or “AAV” as used herein refers to members of the dependovirus genus comprising any particle, sequence, gene, protein, or component derived therefrom.

AAV Particle: As used herein, an “AAV particle” is a virus which includes a capsid and a viral genome with at least one payload region and at least one ITR region. AAV particles of the present disclosure may be produced recombinantly and may be based on adeno-associated virus (AAV) parent or reference sequences. AAV particle may be derived from any serotype, described herein or known in the art, including combinations of serotypes (i.e., “pseudotyped” AAV) or from various genomes (e.g., single stranded or self-complementary). In addition, the AAV particle may be replication defective and/or targeted.

Activity: As used herein, the term “activity” refers to the condition in which things are happening or being done. Compositions of the present disclosure may have activity and this activity may involve one or more biological events.

Administering: As used herein, the term “administering” refers to providing a pharmaceutical agent or composition to a subject.

Administered in combination: As used herein, the term “administered in combination” or “combined administration” means that two or more agents are administered to a subject at the same time or within an interval such that there may be an overlap of an effect of each agent on the patient. In certain embodiments, they are administered within about 60, 30, 15, 10, 5, or 1 minute of one another. In certain embodiments, the administrations of the agents are spaced sufficiently closely together such that a combinatorial (e.g., a synergistic) effect is achieved.

Amelioration: As used herein, the term “amelioration” or “ameliorating” refers to a lessening of severity of at least one indicator of a condition or disease. For example, in the context of neurodegeneration disorder, amelioration includes the reduction of neuron loss.

Animal: As used herein, the term “animal” refers to any member of the animal kingdom. In certain embodiments, “animal” refers to humans at any stage of development. In certain embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In certain embodiments, animals include, but are not limited to, mammals, birds, reptiles, amphibians, fish, and worms. In certain embodiments, the animal is a transgenic animal, genetically-engineered animal, or a clone.

Antisense strand: As used herein, the term “the antisense strand” or “the first strand” or “the guide strand” of a siRNA molecule refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process.

Approximately: As used herein, the term “approximately” or “about,” as applied to one or more values of interest, refers to a value that is similar to a stated reference value. In certain embodiments, the term “approximately” refers to a range of values that fall within 25%, 20%, 19%, 18%, 17%, 16%, 15%, 14%, 13%, 12%, 11%, 10%, 9%, 8%, 7%, 6%, 5%. 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization-based connectivity sufficiently stable such that the “associated” entities remain physically associated.

Baculoviral expression vector (BEV): As used herein a BEV is a baculoviral expression vector, i.e., a polynucleotide vector of baculoviral origin. Systems using BEVs are known as baculoviral expression vector systems (BEVSs).

mBEV or modified BEV As used herein, a modified BEV is an expression vector of baculoviral origin which has been altered from a starting BEV (whether wild type or artificial) by the addition and/or deletion and/or duplication and/or inversion of one or more: genes; gene fragments; cleavage sites; restriction sites; sequence regions; sequence(s) encoding a payload or gene of interest: or combinations of the foregoing.

Bifunctional: As used herein, the term “bifunctional” refers to any substance, molecule or moiety which is capable of or maintains at least two functions. The functions may affect the same outcome or a different outcome. The structure that produces the function may be the same or different.

BIIC: As used herein a BIIC is a baculoviral infected insect cell.

Biocompatible: As used herein, the term “biocompatible” means compatible with living cells, tissues, organs or systems posing little to no risk of injury, toxicity or rejection by the immune system.

Biodegradable: As used herein, the term “biodegradable” means capable of being broken down into innocuous products by the action of living things.

Biologically active: As used herein, the phrase “biologically active” refers to a characteristic of any substance that has activity in a biological system and/or organism. For instance, a substance that, when administered to an organism, has a biological effect on that organism, is considered to be biologically active. In particular embodiments, an AAV particle of the present disclosure may be considered biologically active if even a portion of the encoded payload is biologically active or mimics an activity considered biologically relevant.

Capsid: As used herein, the term “capsid” refers to the protein shell of a virus particle.

Codon optimized: As used herein, the terms “codon optimized” or “codon optimization” refers to a modified nucleic acid sequence which encodes the same amino acid sequence as a parent/reference sequence, but which has been altered such that the codons of the modified nucleic acid sequence are optimized or improved for expression in a particular system (such as a particular species or group of species). As a non-limiting example, a nucleic acid sequence which includes an AAV capsid protein can be codon optimized for expression in insect cells or in a particular insect cell such Spodoptera frugiperda cells. Codon optimization can be completed using methods and databases known to those in the art.

Complementary and substantially complementary: As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present disclosure, the ability to substitute a T is implied, unless otherwise stated. Perfect complementarity or 100% complementarity refers to the situation in which each nucleotide unit of one polynucleotide strand can form hydrogen bond with a nucleotide unit of a second polynucleotide strand. Less than perfect complementarity refers to the situation in which some, but not all, nucleotide units of two strands can form hydrogen bond with each other. For example, for two 20-mers, if only two base pairs on each strand can form hydrogen bond with each other, the polynucleotide strands exhibit 10% complementarity. In the same example, if 18 base pairs on each strand can form hydrogen bonds with each other, the polynucleotide strands exhibit 90% complementarity. As used herein, the term “substantially complementary” means that the siRNA has a sequence (e.g., in the antisense strand) which is sufficient to bind the desired target mRNA, and to trigger the RNA silencing of the target mRNA.

Compound: Compounds of the present disclosure include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

The compounds and salts of the present disclosure can be prepared in combination with solvent or water molecules to form solvates and hydrates by routine methods.

Conditionally active: As used herein, the term “conditionally active” refers to a mutant or variant of a wild-type polypeptide, wherein the mutant or variant is more or less active at physiological conditions than the parent polypeptide. Further, the conditionally active polypeptide may have increased or decreased activity at aberrant conditions as compared to the parent polypeptide. A conditionally active polypeptide may be reversibly or irreversibly inactivated at normal physiological conditions or aberrant conditions.

Conserved: As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In certain embodiments, two or more sequences are said to be “completely conserved” if they are 100% identical to one another. In certain embodiments, two or more sequences are said to be “highly conserved” if they are at least 70% identical, at least 80% identical, at least 90% identical, or at least 95% identical to one another. In certain embodiments, two or more sequences are said to be “highly conserved” if they are about 70% identical, about 80% identical, about 90% identical, about 95%, about 98%, or about 99% identical to one another. In certain embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In certain embodiments, two or more sequences are said to be “conserved” if they are about 30% identical, about 40% identical, about 50% identical, about 60% identical, about 70% identical, about 80/o identical, about 90% identical, about 95% identical, about 98% identical, or about 99% identical to one another. Conservation of sequence may apply to the entire length of an polynucleotide or polypeptide or may apply to a portion, region or feature thereof.

Control Elements: As used herein, “control elements”, “regulatory control elements” or “regulatory sequences” refers to promoter regions, polyadenylation signals, transcription termination sequences, upstream regulatory domains, origins of replication, internal ribosome entry sites (“IRES”), enhancers, and the like, which provide for the replication, transcription and translation of a coding sequence in a recipient cell. Not all of these control elements need always be present as long as the selected coding sequence is capable of being replicated, transcribed and/or translated in an appropriate host cell.

Controlled Release: As used herein, the term “controlled release” refers to a pharmaceutical composition or compound release profile that conforms to a particular pattern of release to effect a therapeutic outcome.

Cytostatic: As used herein, “cytostatic” refers to inhibiting, reducing, suppressing the growth, division, or multiplication of a cell (e.g., a mammalian cell (e.g., a human cell)), bacterium, virus, fungus, protozoan, parasite, prion, or a combination thereof.

Cytotoxic: As used herein, “cytotoxic” refers to killing or causing injurious, toxic, or deadly effect on a cell (e.g., a mammalian cell (e.g., a human cell)), bacterium, virus, fungus, protozoan, parasite, prion, or a combination thereof.

Delivery: As used herein, “delivery” refers to the act or manner of delivering an AAV particle, a compound, substance, entity, moiety, cargo or payload.

Delivery Agent: As used herein, “delivery agent” refers to any substance which facilitates, at least in part, the in vivo delivery of an AAV particle to targeted cells.

Destabilized: As used herein, the term “destable,” “destabilize,” or “destabilizing region” means a region or molecule that is less stable than a starting, wild-type or native form of the same region or molecule.

Detectable label: As used herein, “detectable label” refers to one or more markers, signals, or moieties which are attached, incorporated or associated with another entity that is readily detected by methods known in the art including radiography, fluorescence, chemiluminescence, enzymatic activity, absorbance and the like. Detectable labels include radioisotopes, fluorophores, chromophores, enzymes, dyes, metal ions, ligands such as biotin, avidin, streptavidin and haptens, quantum dots, and the like. Detectable labels may be located at any position in the peptides or proteins disclosed herein. They may be within the amino acids, the peptides, or proteins, or located at the N- or C-termini.

Digest: As used herein, the term “digest” means to break apart into smaller pieces or components. When referring to polypeptides or proteins, digestion results in the production of peptides.

Distal: As used herein, the term “distal” means situated away from the center or away from a point or region of interest.

Dosing regimen: As used herein, a “dosing regimen” is a schedule of administration or physician determined regimen of treatment, prophylaxis, or palliative care.

Encapsulate: As used herein, the term “encapsulate” means to enclose, surround or encase.

Engineered: As used herein, embodiments of the present disclosure are “engineered” when they are designed to have a feature or property, whether structural or chemical, that varies from a starting point, wild type or native molecule.

Effective Amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Feature: As used herein, a “feature” refers to a characteristic, a property, or a distinctive element.

Formulation: As used herein, a “formulation” includes at least one AAV particle and a delivery agent or excipient.

Fragment: A “fragment,” as used herein, refers to a portion. For example, fragments of proteins may include polypeptides obtained by digesting full-length protein isolated from cultured cells.

Functional: As used herein, a “functional” biological molecule is a biological molecule in a form in which it exhibits a property and/or activity by which it is characterized.

Gene expression: The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

Homology: As used herein, the term “homology” refers to the overall relatedness between polymeric molecules, e.g., between polynucleotide molecules (e.g. DNA molecules and/or RNA molecules) and/or between polypeptide molecules. In certain embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the present disclosure, two polynucleotide sequences are considered to be homologous if the polypeptides they encode are at least about 50%, 60%, 70%, 80%, 90%, 95%, or even 99% for at least one stretch of at least about 20 amino acids. In certain embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the present disclosure, two protein sequences are considered to be homologous if the proteins are at least about 50%, 60%, 70%, 80%, or 90% identical for at least one stretch of at least about 20 amino acids.

Heterologous Region: As used herein the term “heterologous region” refers to a region which would not be considered a homologous region.

Homologous Region: As used herein the term “homologous region” refers to a region which is similar in position, structure, evolution origin, character, form or function.

Identity As used herein, the term “identity” refers to the overall relatedness between polymeric molecules, e.g., between polynucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part 1, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman. D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically, a reduction in the level of an mRNA results in a reduction in the level of a polypeptide translated therefrom. The level of expression may be determined using standard techniques for measuring mRNA or protein.

In vitro: As used herein, the term “n vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In certain embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Substantially isolated: By “substantially isolated” is meant that a substance is substantially separated from the environment in which it was formed or detected. Partial separation can include, for example, a composition enriched in the substance or AAV particles of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art.

Linker: As used herein “linker” refers to a molecule or group of molecules which connects two molecules. A linker may be a nucleic acid sequence connecting two nucleic acid sequences encoding two different polypeptides. The linker may or may not be translated. The linker may be a cleavable linker.

MicroRNA (miRNA) binding site: As used herein, a microRNA (miRNA) binding site represents a nucleotide location or region of a nucleic acid transcript to which at least the “seed” region of a miRNA binds.

Modified: As used herein “modified” refers to a changed state or structure of a molecule of the present disclosure. Molecules may be modified in many ways including chemically, structurally, and functionally. As used herein, embodiments of the disclosure are “modified” when they have or possess a feature or property, whether structural or chemical, that varies from a starting point, wild type or native molecule.

Mutation: As used herein, the term “mutation” refers to any changing of the structure of a gene, resulting in a variant (also called “mutant”) form that may be transmitted to subsequent generations. Mutations in a gene may be caused by the alternation of single base in DNA, or the deletion, insertion, or rearrangement of larger sections of genes or chromosomes.

Naturally Occurring: As used herein, “naturally occurring” or “wild-type” means existing in nature without artificial aid, or involvement of the hand of man.

Neurodegeneration: As used herein, the term “neurodegeneration” refers to a pathologic state which results in neural cell death. A large number of neurological disorders share neurodegeneration as a common pathological state. For example, Alzheimer's disease, Parkinson's disease, Huntington's disease, and amyotrophic lateral sclerosis (ALS) all cause chronic neurodegeneration, which is characterized by a slow, progressive neural cell death over a period of several years, whereas acute neurodegeneration is characterized by a sudden onset of neural cell death as a result of ischemia, such as stroke, or trauma, such as traumatic brain injury, or as a result of axonal transection by demyelination or trauma caused, for example, by spinal cord injury or multiple sclerosis. In some neurological disorders, mainly one type of neuronal cell is degenerative, for example, medium spiny neuron degeneration in early HD.

Non-human vertebrate: As used herein, a “non-human vertebrate” includes all vertebrates except Homo sapiens, including wild and domesticated species. Examples of non-human vertebrates include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

Nucleic Acid: As used herein, the term “nucleic acid”, “polynucleotide” and ‘oligonucleotide” refer to any nucleic acid polymers composed of either polydeoxyribonucleotides (containing 2-deoxy-D-ribose), or polyribonucleotides (containing D-ribose), or any other type of polynucleotide which is an N glycoside of a purine or pyrimidine base, or modified purine or pyrimidine bases. There is no intended distinction in length between the term “nucleic acid”, “polynucleotide” and “oligonucleotide”, and these terms will be used interchangeably. These terms refer only to the primary structure of the molecule. Thus, these terms include double- and single-stranded DNA, as well as double- and single stranded RNA.

Off-target: As used herein, “off target” refers to any unintended effect on any one or more target, gene, or cellular transcript.

Open reading frame: As used herein, “open reading frame” or “ORF” refers to a sequence which does not contain a stop codon within the given reading frame, other than at the end of the reading frame.

Operably linked: As used herein, the phrase “operably linked” refers to a functional connection between two or more molecules, constructs, transcripts, entities, moieties or the like.

Patient: As used herein, “patient” refers to a subject who may seek or be in need of treatment, requires treatment, is receiving treatment, will receive treatment, or a subject who is under care by a trained professional for a particular disease or condition.

Payload: As used herein, “payload” or “payload region” refers to one or more polynucleotides or polynucleotide regions encoded by or within a viral genome or an expression product of such polynucleotide or polynucleotide region, e.g., a transgene, a polynucleotide encoding a polypeptide or multi-polypeptide, or a modulatory nucleic acid or regulatory nucleic acid.

Payload construct: As used herein, “payload construct” is one or more vector construct which includes a polynucleotide region encoding or comprising a payload that is flanked on one or both sides by an inverted terminal repeat (ITR) sequence. The payload construct presents a template that is replicated in a viral production cell to produce a therapeutic viral genome.

Payload construct vector: As used herein, “payload construct vector” is a vector encoding or comprising a payload construct, and regulatory regions for replication and expression of the payload construct in bacterial cells.

Payload construct expression vector: As used herein, a “payload construct expression vector” is a vector encoding or comprising a payload construct and which further comprises one or more polynucleotide regions encoding or comprising components for viral expression in a viral replication cell.

Peptide: As used herein, “peptide” is less than or equal to 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile can be used. Lists of suitable salts are found in Remington's Pharmaceutical Sciences, 17^{th} ed., Mack Publishing Company. Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Pharmaceutically acceptable solvate: The term “pharmaceutically acceptable solvate,” as used herein, means a compound of the present disclosure wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

Pharmacokinetic: As used herein, “pharmacokinetic” refers to any one or more properties of a molecule or compound as it relates to the determination of the fate of substances administered to a living organism. Pharmacokinetics is divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body: (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Physicochemical: As used herein, “physicochemical” means of or relating to a physical and/or chemical property.

Preventing: As used herein, the term “preventing” or “prevention” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Proliferate: As used herein, the term “proliferate” means to grow, expand or increase or cause to grow, expand or increase rapidly. “Proliferative” means having the ability to proliferate. “Anti-proliferative” means having properties counter to or inapposite to proliferative properties.

Prophylactic: As used herein, “prophylactic” refers to a therapeutic or course of action used to prevent the spread of disease.

Prophylaxis: As used herein, a “prophylaxis” refers to a measure taken to maintain health and prevent the spread of disease.

Protein of interest: As used herein, the terms “proteins of interest” or “desired proteins” include those provided herein and fragments, mutants, variants, and alterations thereof.

Proximal: As used herein, the term “proximal” means situated nearer to the center or to a point or region of interest.

Purified: As used herein, “purify,” “purified,” “purification” means to make substantially pure or clear from unwanted components, material defilement, admixture or imperfection. “Purified” refers to the state of being pure. “Purification” refers to the process of making pure.

Region: As used herein, the term “region” refers to a zone or general area. In certain embodiments, when referring to a protein or protein module, a region may include a linear sequence of amino acids along the protein or protein module or may include a three-dimensional area, an epitope and/or a cluster of epitopes. In certain embodiments, regions include terminal regions. As used herein, the term “terminal region” refers to regions located at the ends or termini of a given agent. When referring to proteins, terminal regions may include N- and/or C-termini. N-termini refer to the end of a protein comprising an amino acid with a free amino group. C-termini refer to the end of a protein comprising an amino acid with a free carboxyl group. N- and/or C-terminal regions may there for include the N- and/or C-termini as well as surrounding amino acids. In certain embodiments, N- and/or C-terminal regions include from about 3 amino acid to about 30 amino acids, from about 5 amino acids to about 40 amino acids, from about 10 amino acids to about 50 amino acids, from about 20 amino acids to about 100 amino acids and/or at least 100 amino acids. In certain embodiments, N-terminal regions may include any length of amino acids that includes the N-terminus but does not include the C-terminus. In certain embodiments, C-terminal regions may include any length of amino acids, which include the C-terminus, but do not include the N-terminus.

In certain embodiments, when referring to a polynucleotide, a region may include a linear sequence of nucleic acids along the polynucleotide or may include a three-dimensional area, secondary structure, or tertiary structure. In certain embodiments, regions include terminal regions. As used herein, the term “terminal region” refers to regions located at the ends or termini of a given agent. When referring to polynucleotides, terminal regions may include 5′ and 3′ termini. 5′ termini refer to the end of a polynucleotide comprising a nucleic acid with a free phosphate group. 3′ termini refer to the end of a polynucleotide comprising a nucleic acid with a free hydroxyl group. 5′ and 3′ regions may there for include the 5′ and 3′ termini as well as surrounding nucleic acids. In certain embodiments, 5′ and 3′ terminal regions include from about 9 nucleic acids to about 90 nucleic acids, from about 15 nucleic acids to about 120 nucleic acids, from about 30 nucleic acids to about 150 nucleic acids, from about 60 nucleic acids to about 300 nucleic acids and/or at least 300 nucleic acids. In certain embodiments, 5′ regions may include any length of nucleic acids that includes the 5′ terminus but does not include the 3′ terminus. In certain embodiments, 3′ regions may include any length of nucleic acids, which include the 3′ terminus, but does not include the 5′ terminus.

RNA or RNA molecule: As used herein, the term “RNA” or “RNA molecule” or “ribonucleic acid molecule” refers to a polymer of ribonucleotides: the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

RNA interfering or RNAi: As used herein, the term “RNA interfering” or “RNAi” refers to a sequence specific regulatory mechanism mediated by RNA molecules which results in the inhibition or interfering or “silencing” of the expression of a corresponding protein-coding gene. RNAi has been observed in many types of organisms, including plants, animals and fingi. RNAi occurs in cells naturally to remove foreign RNAs (e.g., viral RNAs). Natural RNAi proceeds via fragments cleaved from free dsRNA which direct the degradative mechanism to other similar RNA sequences. RNAi is controlled by the RNA-induced silencing complex (RISC) and is initiated by short/small dsRNA molecules in cell cytoplasm, where they interact with the catalytic RISC component argonaute. The dsRNA molecules can be introduced into cells exogenously. Exogenous dsRNA initiates RNAi by activating the ribonuclease protein Dicer, which binds and cleaves dsRNAs to produce double-stranded fragments of 21-25 base pairs with a few unpaired overhang bases on each end. These short double stranded fragments are called small interfering RNAs (siRNAs).

Sample: As used herein, the term “sample” or “biological sample” refers to a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

Self-complementary viral particle: As used herein, a “self-complementary viral particle” is a particle included of at least two components, a protein capsid and a polynucleotide sequence encoding a self-complementary genome enclosed within the capsid.

Sense Strand: As used herein, the term “the sense strand” or “the second strand” or “the passenger strand” of a siRNA molecule refers to a strand that is complementary to the antisense strand or first strand. The antisense and sense strands of a siRNA molecule are hybridized to form a duplex structure. As used herein, a “siRNA duplex” includes a siRNA strand having sufficient complementarity to a section of about 10-50 nucleotides of the mRNA of the gene targeted for silencing and a siRNA strand having sufficient complementarity to form a duplex with the other siRNA strand.

Short interfering RNA or siRNA: As used herein, the terms “short interfering RNA,” “small interfering RNA” or “siRNA” refer to an RNA molecule (or RNA analog) comprising between about 5-60 nucleotides (or nucleotide analogs) which is capable of directing or mediating RNAi. In certain embodiments, a siRNA molecule includes between about 15-30 nucleotides or nucleotide analogs, such as between about 16-25 nucleotides (or nucleotide analogs), between about 18-23 nucleotides (or nucleotide analogs), between about 19-22 nucleotides (or nucleotide analogs) (e.g., 19, 20, 21 or 22 nucleotides or nucleotide analogs), between about 19-25 nucleotides (or nucleotide analogs), and between about 19-24 nucleotides (or nucleotide analogs). The term “short” siRNA refers to a siRNA comprising 5-23 nucleotides, such as 21 nucleotides (or nucleotide analogs), for example, 19, 20, 21 or 22 nucleotides. The term “long” siRNA refers to a siRNA comprising 24-60 nucleotides, such as about 24-25 nucleotides, for example, 23, 24, 25 or 26 nucleotides. Short siRNAs may, in some instances, include fewer than 19 nucleotides, e.g., 16, 17 or 18 nucleotides, or as few as 5 nucleotides, provided that the shorter siRNA retains the ability to mediate RNAi. Likewise, long siRNAs may, in some instances, include more than 26 nucleotides, e.g., 27, 28, 29, 30, 35, 40, 45, 50, 55, or even 60 nucleotides, provided that the longer siRNA retains the ability to mediate RNAi or translational repression absent further processing, e.g., enzymatic processing, to a short siRNA. siRNAs can be single stranded RNA molecules (ss-siRNAs) or double stranded RNA molecules (ds-siRNAs) comprising a sense strand and an antisense strand which hybridized to form a duplex structure called siRNA duplex.

Signal Sequences: As used herein, the phrase “signal sequences” refers to a sequence which can direct the transport or localization of a protein.

Single unit dose: As used herein, a “single unit dose” is a dose of any therapeutic administered in one dose/at one time/single route/single point of contact. i.e., single administration event. In certain embodiments, a single unit dose is provided as a discrete dosage form (e.g., a tablet, capsule, patch, loaded syringe, vial, etc.).

Similarity. As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Split dose: As used herein, a “split dose” is the division of single unit dose or total daily dose into two or more doses.

Stable: As used herein “stable” refers to a compound that is sufficiently robust to survive isolation to a useful degree of purity from a reaction mixture, and in certain embodiments, capable of formulation into an efficacious therapeutic agent.

Stabilized: As used herein, the term “stabilize”, “stabilized,” “stabilized region” means to make or become stable.

Subject: As used herein, the term “subject” or “patient” refers to any organism to which a composition in accordance with the present disclosure may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used herein and as it relates to plurality of doses, the term means within 2 seconds.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In certain embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In certain embodiments, an individual who is susceptible to a disease, disorder, and/or condition will develop the disease, disorder, and/or condition. In certain embodiments, an individual who is susceptible to a disease, disorder, and/or condition will not develop the disease, disorder, and/or condition.

Sustained release: As used herein, the term “sustained release” refers to a pharmaceutical composition or compound release profile that conforms to a release rate over a specific period of time.

Synthetic: The term “synthetic” means produced, prepared, and/or manufactured by the hand of man. Synthesis of polynucleotides or polypeptides or other molecules of the present disclosure may be chemical or enzymatic.

Targeting: As used herein, “targeting” means the process of design and selection of nucleic acid sequence that will hybridize to a target nucleic acid and induce a desired effect.

Targeted Cells: As used herein, “targeted cells” refers to any one or more cells of interest. The cells may be found in vitro, in vivo, in situ or in the tissue or organ of an organism. The organism may be an animal, such as a mammal, a human, or a human patient.

Terminal region: As used herein, the term “terminal region” refers to a region on the 5′ or 3′ end of a region of linked nucleosides or amino acids (polynucleotide or polypeptide, respectively).

Terminally optimized: The term “terminally optimized” when referring to nucleic acids means the terminal regions of the nucleic acid are improved in some way, e.g., codon optimized, over the native or wild type terminal regions.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In certain embodiments, a therapeutically effective amount is provided in a single dose. In certain embodiments, a therapeutically effective amount is administered in a dosage regimen comprising a plurality of doses. Those skilled in the art will appreciate that in certain embodiments, a unit dosage form may be considered to include a therapeutically effective amount of a particular agent or entity if it includes an amount that is effective when administered as part of such a dosage regimen.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in 24-hour period. It may be administered as a single unit dose.

Transfection: As used herein, the term “transfection” refers to methods to introduce exogenous nucleic acids into a cell. Methods of transfection include, but are not limited to, chemical methods, physical treatments and cationic lipids or mixtures.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Vector: As used herein, a “vector” is any molecule or moiety which transports, transduces or otherwise acts as a carrier of a heterologous molecule. Vectors of the present disclosure may be produced recombinantly and may be based on and/or may include adeno-associated virus (AAV) parent or reference sequence. Such parent or reference AAV sequences may serve as an original, second, third or subsequent sequence for engineering vectors. In non-limiting examples, such parent or reference AAV sequences may include any one or more of the following sequences: a polynucleotide sequence encoding a polypeptide or multi-polypeptide, which sequence may be wild-type or modified from wild-type and which sequence may encode full-length or partial sequence of a protein, protein domain, or one or more subunits of a protein; a polynucleotide comprising a modulatory or regulatory nucleic acid which sequence may be wild-type or modified from wild-type; and a transgene that may or may not be modified from wild-type sequence. These AAV sequences may serve as either the “donor” sequence of one or more codons (at the nucleic acid level) or amino acids (at the polypeptide level) or “acceptor” sequences of one or more codons (at the nucleic acid level) or amino acids (at the polypeptide level).

Viral genome: As used herein, a “viral genome” or “vector genome” or “viral vector” refers to the nucleic acid sequence(s) encapsulated in an AAV particle. Viral genomes comprise at least one payload region encoding polypeptides or fragments thereof.

### VI. Equivalents and Scope

Those skilled in the art will recognize or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

### Examples

### Example 1. Downstream—Cell Lysis

A bulk harvest pool of AAV particles was producing using Baculovirus-production systems of the present disclosure, with S/9 insect cells used as AAV viral production cells.

Chemical Lysis was initiated on the bulk harvest in the Production Bioreactor by adding 0.2M Arginine HCl, 0.25% w/v Triton X-100 surfactant, 10 U/mL Benzonase nuclease, and finally 2 M Tris Base to provide a lysis pH of 6.7-7.3. The lysis mixture was held at 37° C. for 4.0-6.0 hours until a crude lysate pool was generated. The crude lysate pool was be brought to room temperature and aseptically sampled prior to further processing.

In one alternative, Chemical Lysis was initiated on the bulk harvest in the Production Bioreactor by adding Arginine HCl and Triton X-100 surfactant with a lysis pH of 6.8-7.5. The lysis mixture was held at 27° C. for 4.0-6.0 hours until a crude lysate pool was generated.

In one alternative, chemical lysis was initiated on the bulk harvest in the Production Bioreactor (225 L working volume) by adding Tris Base to provide a lysis pH of 6.9-7.1, followed by adding Arginine HCl, Sartorius Denarase nuclease, and finally Triton X-100 surfactant in PBS background. The lysis mixture was held at 37° C. for 3-4 hours until a crude lysate pool was generated.

In one alternative, chemical lysis was initiated on the bulk harvest in the Production Bioreactor by adding Triton X-100 surfactant and Benzonase nuclease. The lysis mixture was held at 37° C. for 6-12 hours with agitation, until a crude lysate pool was generated.

### Example 2. Lysis Solutions Studies

### Lysis Agent Study

Lysis agents were studied to identify specific agents which could provide unexpected and improved environmental safety, lysis efficiency, filtration throughputs and product yields. 44 lysis agents were tested, including nonionic detergents, ionic detergents, and zwitterionic detergents. The study also included three PBS mixtures as positive controls and lysis solvents as negative controls.

AAV particles were produced using sf9 viral production cells according to the methods and systems of the present disclosure. Lysis studies were then conducted under the following conditions: Target final detergent concentration of 0.5% w/v; 20 mL experimental scale; and lysis incubation at 27° C. for 4 hours. Recovery yield of the AAV particle product was measured using ddPCR. Throughput of the AAV particle product was measured using 0.22 μm filter throughput. Triton X-100 (0.5% w/v target final concentration) % vas used as the Reference Standard for relative comparison of the results.

The lysis agents and corresponding results from this study are summarized below in Table 4 (data for Relative Throughput and Relative Yield are percentages relative to Triton X-100 Reference):

TABLE 4
Lysis Agent Study Results

| | | | Filter | Relative | ddPCR Yield | Relative |
|---|---|---|---|---|---|---|
| Identifier | Lysis Agent | Type | Throughput (g) | Throughput (%) | (vg/mL, 10¹¹) | Yield (%) |
| 1 | Cetyltrimethylammonium | Caltionic | 19.28 | 192.6 | 3.61 | 181.54 |
| | Bromide (CTAB) | | | | | |
| 2 | EMPIGEN BB | Zwitterionic | 19.11 | 190.9 | 3.99 | 198.88 |
| | Detergent (30% w/w) | | | | | |
| 3 | Zwittergent 3-12 | Zwitterionic | 19.07 | 190.5 | 2.61 | 129.83 |
| 4 | Lauryldimethyllamine | Zwitterionic | 19.02 | 190.0 | 2.70 | 133.95 |
| | N-oxide (LDAO, 30% w/w) | | | | | |
| 5 | Zwittergent 3-14 | Zwitterionic | 19 | 189.8 | 3.04 | 150.66 |
| 6 | Tomadol 1200 | Nonionic | 13.3 | 132.9 | 3.93 | 136.34 |
| 7 | Sodium Dodecyl | Anionic | 13.09 | 130.8 | 4.06 | 138.62 |
| | Sulfate (10% w/w) | | | | | |
| 8 | Triton X-100 (10% w/v) | Nonionic | 10.74 | 107.3 | 3.48 | 97.49 |
| 9 | Igepal CA-630 | Nonionic | 10.69 | 106.8 | 3.97 | 110.70 |
| | (Nonidet P-40) | | | | | |
| 10 | Triton X-10) (10% w/v) | Nonionic | 10.01 | 100.0 | 3.83 | 100.00 |
| 11 | Lutensol XL 90 | Nonionic | 9.79 | 97.8 | 3.64 | 92.95 |
| 12 | Tomadol 900 | Nonionic | 9.65 | 96.4 | 3.77 | 94.89 |
| 13 | Brij 35 (10% w/v) | Nonionic | 9.37 | 93.6 | 3.56 | 87.01 |
| 14 | Tergitol TMN-100X | Nonionic | 9.29 | 92.8 | 3.73 | 90.38 |
| | (90% w/v) | | | | | |
| 15 | CHAPS | Zwitterionic | 8.76 | 87.5 | 2.85 | 65.12 |
| 16 | Tergitol NP-10 | Nonionic | 8.24 | 82.3 | 3.72 | 79.95 |
| 17 | PBS, pH 7.4 (Control 1A) | Control | 8.21 | 82.0 | 3.55 | 76.07 |
| 18 | PBS, pH 7.4 (Control 1B) | Control | 8.08 | 80.7 | 3.88 | 81.77 |
| 19 | Brij 58(10% w/v ) | Nonionic | 8.07 | 80.1 | 3.29 | 68.82 |
| 20 | Lutensol XP 90 | Nonionic | 7.86 | 78.5 | 3.43 | 70.32 |
| 21 | Zwittergent 3-10 | Zwittenonic | 7.85 | 78.4 | 3.71 | 75.96 |
| 22 | CHAPSO | Zwitterionic | 7.82 | 78.1 | 3.45 | 70.37 |
| 23 | Sodium Taurocholate | Anionic | 7.81 | 78.0 | 2.77 | 56.43 |
| 24 | GENAPOL X-I00 | Nonionic | 7.32 | 73.1 | 1.88 | 35.90 |
| | (10% w/w) | | | | | |
| 25 | Tergitol 15-S-40 (70% w/v) | Nonionic | 6.93 | 69.2 | 2.22 | 40.13 |
| 26 | NP-40 (10% w/v) | Nonionic | 6.75 | 67.4 | 3.51 | 61.80 |
| 27 | Tergnol TMN-6 (90% w/v) | Nonionic | 6.67 | 66.6 | 3.34 | 58.11 |
| 28 | Tergitol NP-9 | Nonionic | 6.63 | 66.2 | 3.77 | 56.55 |
| 29 | Tween-80 (10% w/v) | Nonionic | 6.39 | 63.8 | 2.05 | 34.17 |
| 30 | PBS, pH 7.4 (−0.2 M | Control | 6.34 | 63.3 | 1.78 | 29.44 |
| | Arginine, 3 mL PBS) | | | | | |
| 31 | Tergitol 15-S-9 | Nonionic | 6.27 | 62.6 | 2.20 | 35.98 |
| 32 | Pluronic F 127 (10% w/v) | Nonionic | 6.26 | 62.5 | 2.40 | 39.19 |
| 33 | Ecosurf EH-9 90% w/v) | Nonionic | 6.01 | 60.0 | 3.94 | 61.76 |
| 34 | Tergitol 15-S-7 | Nonionic | 5.85 | 58.4 | 2.86 | 43.64 |
| 35 | Safe Care 1000 (SC-1000) | Nonionic | 5.85 | 58.4 | 3.47 | 52.95 |
| 36 | Pluronic F68 (10%w/v) | Nonionic | 5.69 | 56.8 | 3.29 | 48.83 |
| 37 | Tween-20 (10% w/v) | Nonionic | 5.66 | 56.5 | 4.06 | 59.94 |
| 38 | Ecosurf SA-9 | Nonionic | 5.54 | 55.3 | 3.86 | 55.78 |
| 39 | Caprylic Acid | Organic Acid | 5.3 | 52.9 | 3.04 | 42.03 |
| | (Octanoic Acid) | | | | | |
| 40 | Tri-n-butyl Phosphate | Solvent | 5.2 | 51.9 | 4.26 | 57.78 |
| | (TnBP) | | | | | |
| 41 | Tomadol 400 | Nonionic | 4.4 | 44.0 | 4.32 | 49.58 |
| 42 | Natsurf 265-LQ-(AP) | Nonionic | 4.16 | 41.6 | 1.70 | 18.45 |
| 43 | APG 325N | Nonionic | 4.03 | 40.3 | 1.43 | 15.03 |
| 44 | Sodium Caprylate | Organic Acid | 3.2 | 32.0 | 2.10 | 17.53 |
| 45 | Triton CG-110 | Nonionic | 3.08 | 30.8 | 1.89 | 15.18 |
| 46 | ELUGENT (50% w/w) | Nonionic | 2.44 | 21.4 | 1.78 | 11.33 |
| 47 | Triton X-114 (10% w/v) | Nonionic | 0.91 | 9.1 | 1.82 | 4.32 |
| 48 | Sodium Deoxycholate | Anionic | 0.81 | 8.1 | 3.89 | 8.22 |

The study results highlight several notable classes of nonionic, ionic, and zwitterionic species that show improved relative filter throughput and a higher product yield.

### Viral Clearance Study

Four lysis agents were studied for their effectiveness at inactivating Baculovirus (BACV) and Vesicular Stomatitis Virus (VSV) within bulk harvest pools of AAV particles produced using Baculovirus-production systems and Sf9 insect cells. BACV is a known process contaminant, and VSV was used as a model for known Rhabdoviral cell line contaminants.

The four lysis agents were as follows: Detergent 1—Lauryldimethylamine N-oxide (LDAO) (MilliporeSigma P/N 40236); Detergent 2—Ecosurf SA-9 (Dow Chemical, MilliporeSigma P/N STS0007); Detergent 3—Empigen BB (Calbiochem, MilliporeSigma P/N 324690); and Detergent 4—Zwittergent 3-14 (Calbiochem, MilliporcSigma P/N 693017).

Results from this viral clearance study are summarized below in Table 5 (Values represent Log₁₀ reduction values for viral contaminant TCID50).

TABLE 5
Viral Clearance Study

| | Baculovirus (BACV) | | | | Vesicular Stomatitis Virus (VSV) | | | |
|---|---|---|---|---|---|---|---|---|
| | Detergent 1 | Detergent 2 | Detergent 3 | Detergent 4 | Detergent 1 | Detergent 2 | Detergent 3 | Detergent 4 |
| 30 | 2.84 | 2.9 | 2.66 | 2.25 | >4.56 | >4.08 | 5.15 | 4.13 |
| minutes | | | | | | | | |
| 60 | 2.90 | 2.9 | 3.62 | 3.09 | >4.56 | >4.08 | 5.39 | 4.55 |
| minutes | | | | | | | | |
| 120 | >5.43 | >5.43 | >6.21 | >4.77 | >5.76 | >5.76 | 6.52 | 6.07 |
| minutes | | | | | | | | |

Study results showed that Detergent 3 (Empigen BB) had the strongest viral BACV and VSV viral clearance activity.

### Arginine Concentration Study

Chemical Lysis was studied using 0.25% Triton X-100 lysis agent at varying pH conditions. Varying concentrations of arginine were also added to the cell culture broth prior to lysis of the harvest pool. Results from this study are summarized below in Table 6.

TABLE 6
Arginine Concentration Study

| | Triton-X 100 | | 0.2 M | 0.5 M | 1 M | AAV Recovery |
|---|---|---|---|---|---|---|
| Sample | (%) | pH | NaCl | Arginine | Arginine | (108 particles/mL) |
| 1 | 0.25 | 3.5 | | | | 3028 |
| 2 | 0.25 | 4.5 | | | | 2823 |
| 3 | 0.25 | 5.5 | | | | 2013 |
| 4 | 0.25 | 6,5 | | | | 1725 |
| 5 | 0.25 | 7.5 | | | | 1615 |
| 6 | 0.25 | 8.5 | | | | 2032 |
| 7 | 0.25 | 5 | X | | | 2698 |
| 8 | 0.25 | 5 | | X | | 4237 |
| 9 | 0.25 | 5 | | | X | 4336 |
| 10 | 0.25 | 6.2 | X | | | 3098 |
| 11 | 0.25 | 6.2 | | X | | 4699 |
| 12 | 0.25 | 6.2 | | | X | 3968 |
| 13 | 0.25 | 7 | X | | | 3914 |
| 14 | 0.25 | 7 | | X | | 5318 |
| 15 | 0.25 | 7 | | | X | 3744 |

Study results showed that the addition of 0.5M arginine to the harvest pool prior to lysis significantly increased the AAV recovery yield from the chemical lysis process, and was most effective at pH between 6.0-7.0.

### Example 3. Downstream—Depth Filtration

A crude lysate pool from the chemical lysis process described in Example 1 was provided. The crude lysate pool was processed through Depth Filtration using an EMD Millipore Millistak⁺ POD filter. A filter recovery flush using 20 mM sodium phosphate, 350 mM sodium chloride and Pluronic F-68 (mixture pH of 7.4) was passed through the depth filter, with the flushed recovery being added to the depth filtered pool.

In one alternative, the crude lysate pool was processed through Depth Filtration using a Millipore MC0SP23CL3 filter.

In one alternative, the recovery flush used 50 mM sodium phosphate. 350 mM sodium chloride and Pluronic F-68 (mixture pH of 7.4). In one alternative, the recovery flush used PBS.

### Example 4. Depth Filtration Study

Depth filtration systems from four (4) vendors (MilliporeSigma. Pall Corporation, 3M, Sartorius) were studied to identify specific systems and filter combinations which could provide unexpected and improved filtration throughputs and product yields.

Filter testing was completed on four depth filters selected from each of the four vendors, in combination with Sartopore 2XLG Sartoscale (5445307GV-LX-C) sterile filters (0.22 μm filters). Sterile filters from EMD Millipore Express SHC (SHGEA25NB6), Pall Supor EKV Membrane (KM2EKVS) and 3M LifeASSURE PDA (70357-03-B-PDA020N). However, Sartopore 2XLG was found to provide superior results and was the select sterile filter for testing.

Data output for the depth filters from the testing included the following: (i) Pmax (increase in delta-pressure across the depth filter at a constant flow): (ii) Recovery Yield (total vector genomes present in the filtrate as a percentage of total vector genomes loaded on the filter); (iii) Turbidity (measured in standard units of NTU); and (iv) Vmax (constant pressure) over in-series sterile grade filters (maximum liters of load that can be filtered per m² of filter area before total plugging, with sterile filter Vmax evaluating how well the upstream depth filter removed particulates).

Data output for the sterile filters from the testing included straight test Vmax (sterile filter only), and in-series testing (sterile filter in-series with depth filter to evaluate how well the upstream depth filter removed particulates).

Vmax Constant Press Testing (Depth Filter Only)—Measurement in the decrease in flow (L/m²) as a function of throughput resulting from particle retention (minimum flowrate endpoint); (ii) (iii) Recovery Yield (Depth Filter+Sterile Filter)—Measurement of product recovery output as a percentage of viral product input, and (iv) Turbidity of output (Depth Filter Only) in Nephelometric Turbidity Units (NTU).

### Stage 1

In Stage 1 of the study, all 16 filter systems were tested (single filter stack) using AAV particle lysate. Results from this study are summarized below in Table 7 (Normalized Recovery percentage are normalized against the highest recovery percentage of 98%).

TABLE 7
Depth Filtration Study - Stage 1 Results

| | | | | | | | Normalized |
|---|---|---|---|---|---|---|---|
| | | Filter | Vmax | Pmax | Turbidity | Recovery | Recovery |
| Vendor | Primary Filter | Identifier | (L/m²) | (PSID) | (NTU) | Yield (%) | Yield (%) |
| Millipore | MC0HC23CL3 | A-1 | 577 | 3.6 | 4.3 | 86 | 88 |
| (A) | MC0SP23CL3 | A-2 | 988 | 1.2 | 3.19 | 83 | 85 |
| | MD0HC23CL3 | A-3 | 500 | 1.2 | 9.08 | 92 | 94 |
| | MD0SP23CL3 | A-4 | 543 | 0.9 | 4.25 | 82 | 84 |
| Pall | SC050PDP8 | B-1 | 301 | 0.8 | 8.55 | 96 | 98 |
| (B) | SC050PDH4 | B-2 | 2492 | 4 | 3.11 | 88 | 90 |
| | SC050PDK7 | B-3 | 507 | 3.2 | 4.06 | 94 | 96 |
| | SC050PDK11 | B-4 | 365 | 2.4 | 11 | 94 | 96 |
| 3M | BC0025L10SP | C-1 | 1037 | 2.8 | 8.93 | 98 | 100 |
| (C) | BC0025L05SP01A | C-2 | 851 | 0.2 | 9.75 | 97 | 99 |
| | BC0025L05SP | C-3 | 1014 | 0.2 | 10.3 | 94 | 96 |
| | BC0025L10SP02A | C-4 | 1093 | 0.4 | 7.28 | 91 | 93 |
| Sartorius | 29CDL60-CACHH-M | D-1 | 5000 | 2 | 4 | 92 | 94 |
| (D) | 29CDL75-CACHH-M | D-2 | 5000 | 0.4 | 5 | 94 | 96 |
| | 29CS200-CACHH | D-3 | 5000 | 7.8 | 4 | 84 | 86 |
| | 29CS400-CACHH | D-4 | 4341 | 1.2 | 8 | 95 | 97 |

### Stage 2

In Stage 2 of the study, one primary filter from Stage 1 was chosen for each vendor. Each of the chosen primary filters was then individually tested (double filter stack) with four secondary depth filters from the same vendor. Results from this study are summarized below in Table 8 (Normalized Recovery percentage are normalized against the highest recovery percentage of 103%).

TABLE 8
Depth Filtration Study - Stage 2 Results

| | | | | | | Normalized |
|---|---|---|---|---|---|---|
| | | Stack | Pmax | Turbidity | Recovery | Recovery Yield |
| Primary Filter | Secondary Fitter | Identifier | (PSID) | (NTU) | Yield (%) | (%) |
| Millipore (A) | MB1HC23CL3 ** | A2-1 | 11 | 7.92 | 82 | 79.6 |
| C0SP | MA1HC23CL3 | A2-2 | 3.5 | 7.69 | 69 | 67.0 |
| | MX0SP23CL3 | A2-3 | 2.5 | 5.64 | 53 | 51.5 |
| | MF0HC23CL3 | A2-4 | 2.2 | 7.15 | 57 | 55.3 |
| Pall (B) | SC050PDD1 | B3-1 | 5 | 6.93 | 78 | 75.7 |
| PDH4 | SC050PDE2 | B3-2 | 25 | 7.37 | 89 | 86.4 |
| | SC050PDL3 | B3-3 | 4 | 7.27 | 87 | 84.5 |
| | SC050PDE2 ** | B3-4 | 25 | 6.97 | 86 | 83.5 |
| 3M (C) | BC0025L60SP05A | C1-1 | 2.5 | 7.92 | 90 | 87.4 |
| 10SP02A | BC0025L60ZB05A | C1-2 | 3 | 8.92 | 69 | 67.0 |
| | BC0025L30SP02 A ** | C1-3 | 0.77 | 8.74 | 103 | 100.0 |
| | BC0025L60SP02A ** | C1-4 | 1.6 | 8.48 | 90 | 87.4 |
| Sartorius (D) | 29CDL20-CACHH-M | D24 | 2.6 | 1..75 | 75 | 72.8 |
| 29CDL75 | 29CDL10-CACHH-M | D2-2 | 2.7 | 1.52 | 74 | 71.8 |
| | 29CS080-CACHH-M | D2-3 | 1.4 | 2.03 | 81 | 78.6 |
| Sartorius S200 | 29CS040-CACHH-M | D3-4 | 0.6 | 1.9 | 64 | 62.1 |

** - Secondary Filter only (No primary Filter)

### Stage 3

In Stage 3 of the study, one combination of primary filter+secondary filter from Stage 2 was chosen for each vendor. Each of the chosen filter combinations was then tested (double filter stack+sterile filter) for recovery yield and turbidity. Results from this study are summarized below in Table 9 (Normalized Recovery percentage are normalized against the highest recovery percentage of 87%).

TABLE 9
Depth Filtration Study - Stage 3 Results

| | Primary | Secondary | Turbidity | Pmax | Recovery | Normalized Recovery |
|---|---|---|---|---|---|---|
| Vendor | Filter | Filter | (NTU) | (PSID) | Yield (%) | Yield (%) |
| Millipore | C0SP | B1HC | 2.61 | 5 | 76 | 87 |
| | | | 2.21 | 1.42 | 79 | 91 |
| Pall | PDH4 | PDD1 | 2.30 | 8.24 | 65 | 74 |
| | | | 2.80 | 22.79 | 67 | 77 |
| 3M | 10SP02A | 60SP05A | 9.95 | 1.29 | S7 | 100 |
| | | | 4.34 | 1.22 | S7 | 100 |
| Sartorius | DL60 | S040 | 2.67 | 1.07 | 69 | 80 |
| | | | 2.66 | 2.86 | 71 | 82 |

### Example 5. Downstream—0.2 μm Filtration

A depth filtered pool from Example 3 was provided. The depth filtered pool from Depth Filtration was processed through 0.2 μm Filtration using an EMD Millipore Express SHC XL150 0.5/0.2 μm filter. A filter recovery flush using 20 mM sodium phosphate, 350 mM sodium chloride and Pluronic F-68 (mixture pH of 7.4) was passed through the 0.2 μm filter, with the flushed recovery being added to the 0.2 μm filtered pool. The resulting 0.2 μm filtered pool is spiked with NaCl and held for 1-2 days to form a clarified lysate pool. The clarified lysate pool was stored at 2-8° C.

In one alternative, the 0.2 μm Filtration used a Sartorius Sartopore 2XLG, 0.8/0.2 μm filter. In another alternative, the 0.2 μm Filtration included a recovery flush using 50 mM sodium phosphate, 350 mM sodium chloride and Pluronic F-68 (mixture pH of 7.4).

### Example 6. Downstream—Affinity Chromatography

A clarified lysate pool from Example 5 was provided. The clarified lysate pool from Depth Filtration and 0.2 μm Filtration was processed through Affinity Chromatography (AFC) using a GE AVB Sepharose HP column resin. The column resin was equilibrated with a mixture of 20 mM sodium phosphate, 350 mM sodium chloride and Pluronic F-68 (mixture pH of 7.4). The column resin was then loaded with the clarified lysate pool at 18-25° C., and then flushed with a mixture of 20 mM sodium phosphate, 350 mM sodium chloride and Pluronic F-68 (mixture pH of 7.4). This was followed by a first wash of the column resin with a mixture of 20 mM sodium citrate, 1M sodium chloride and Pluronic F-68 (mixture pH of 6.0); and a second wash of the column resin with a mixture of 10 mM sodium citrate, 350 mM sodium chloride and Pluronic F-68 (mixture pH of 6.0). The filtered product was then eluted from the column resin using a mixture of 20 mM sodium citrate, 350 mM sodium chloride and Pluronic F-68 (mixture pH of 3.0).

The resulting elution pool was neutralized with 0.5 M Tris Base and Pluronic F-68. The neutralized elution pool was then processed through 0.2 μm Filtration using an EMD Millipore Express SHC XL6000 0.5/0.2 μm filter, resulting in an AFC pool (also referred to as an AVB pool) with a working pool volume of 8.5-9.0 L.

In one alternative, the column resin was equilibrated and flushed with a mixture of 50 mM sodium phosphate, 350 mM sodium chloride and Pluronic F-68 (mixture pH of 7.4). In one alternative, the column resin was not flushed before the first and second wash steps. In another alternative, the resulting elution pool was neutralized with 2 M Tris Base and Pluronic F-68.

### Example 7. Affinity Chromatography (AFC) Regeneration/Cycling Study

Affinity Chromatography (AFC) regeneration was studied to identify specific regeneration agents which could provide unexpected and improved AVB regeneration and cycling.

In Stage 1, AFC regeneration and cycling was analyzed for legacy strip agent (350 mM NaCl, 20 mM Citrate, 0.001% Pluronic F-68, pH 2.5), using 20 successive runs and regeneration cycles, with Elution Peak AUC (mL× mAU) being measured after every regeneration. After 5 regeneration cycles. AFC columns being regenerated using legacy strip agent were down to 70% Elution Peak AUC from original; Elution Peak AUC was down to 27% after 10 runs, down to 25% after 15 runs, and then maintained at around 25% Elution Peak AUC through 20 runs.

In Stage 2, AFC regeneration was studied using four AFC Strip Agents under 6 successive AFC regeneration cycles. Results from this study are summarized below in Table 10.

TABLE 10
AFC Regeneration Study

| Strip | | | | Run 6/ |
|---|---|---|---|---|
| Agent | | Run 1 AUC | Run 6 AUC | Run 1 |
| Identifier | Strip Agent | (mL*mAU) | (mL*mAU) | (%) |
| 1 | 10% v/v Isopropanol | 234.6 | 142.9 | 61% |
| 2 | 10 mM NaOH | 243.8 | 194.4 | 80% |
| 3 | 2M Guanidine HCl | 249 | 237 | 95% |
| 4 | 2M Urea | 247.9 | 157.6 | 64% |

In Stage 3, AFC regeneration was studied using 2M Guanidine HCl under 12 successive AFC regeneration cycles. Results from this study are summarized below in Table 11.

TABLE H
2M Guanidine HCl Regeneration Study

| | AUC | % of |
|---|---|---|
| Run # | (mL*mAU) | Run 1 |
| 1 | 240.5 | 100% |
| 5 | 231.7 | 96% |
| 10 | 229.8 | 96% |
| 12 | 185.8 | 77% |

This study showed that 2M Guanidine HCl provided unexpected and improved AFC regeneration and cycling results, as it was able to maintain above 95% Elution Peak AUC after 10 successive AVB regeneration cycles and above 75% Elution Peak AUC after 12 successive AFC regeneration cycles.

### Example 8. Downstream—Ion-Exchange Chromatography

A neutralized AFC pool from Example 6 was provided. The AFC pool was processed through Anion-Exchange Chromatography (AEX) using a Sartorius Sartobind Q Membrane (bind-and-elute mode). The AEX membrane was equilibrated with a first mixture of 20 mM Tris, 2 M sodium chloride and Pluronic F-68 (mixture pH of 8.0), and then a second mixture of 20 mM Tris, 100 mM sodium chloride and Pluronic F-68 (mixture pH of 8.0). The AEX membrane system was then loaded with the AFC pool at 18-25° C. The system was flushed with a mixture of 20 mM Tris, 100 mM sodium chloride and Pluronic F-68 (mixture pH of 8.0). The product was then eluted from the AEX membrane system with a mixture of 20 mM Tris, 220 mM sodium chloride and Pluronic F-68 (mixture pH of 8.0), with the entire elution being collected. The AEX elution pool was then processed through 0.2 μm Filtration using an EMD Millipore Express SHCXL150 filter, resulting in an AEX pool with a working pool volume of 1.5-2.0 L.

In one alternative, the neutralized AFC pool was processed through AEX using a Millipore Fractogel TMAE HiCap(m) Flow-Through membrane resin. The AEX membrane was charged and equilibrated with a first mixture of 20 mM Tris, 2 M sodium chloride and Pluronic F-68 (mixture pH of 8.0), and then a second mixture of 40 mM Tris, 170 mM sodium chloride and Pluronic F-68 (mixture pH of 8.5). The AEX membrane system was then loaded with the AFC pool at 18-25° C. The system was flushed and eluted with a mixture of 40 mM Tris, 170 mM sodium chloride and Pluronic F-68 (mixture pH of 8.5), with the entire elution being collected. The AEX elution pool was then processed through 0.2 μm Filtration using an EMD Millipore Express SHCXL150 filter, resulting in an AEX pool.

In one alternative, the neutralized AFC pool was processed through AEX using a GE Q Sepharose HP membrane resin. The AEX membrane was equilibrated with a mixture of 50 mM Bis-Tris Propane, 200 mM sodium chloride and Pluronic F-68 (mixture pH of 9.0). The AEX membrane system was loaded with the AFC pool at 18-25° C. and 150 cm/hr. The system was flushed and eluted with a mixture of 50 mM Bis-Tris Propane, 200 mM sodium chloride and Pluronic F-68 (mixture pH of 9.0). The AEX elution pool was neutralized with a mixture of Tris, NaCl, and Pluronic F-68 (mixture pH of pH 7.5). The AEX elution pool was then processed through 0.2 μm Filtration using an EMD Millipore Express SH CXL 150 filter, resulting in an AEX pool.

In one alternative, the neutralized AFC pool was processed through Anion-Exchange Chromatography (AEX) using a Poros HQ membrane resin.

In one alternative, the neutralized AFC pool was processed through Cation-Exchange Chromatography (CEX) using a Poros XS membrane resin. The CEX membrane was charged with 1 M NaCl, then equilibrated with 20 mM Tris, 100 mM NaCl, and Pluronic F-68 (mixture pH of pH 8.5). The CEX membrane system was then loaded with the AFC pool. The system was washed with 20 mM Tris and Pluronic F-68 (mixture pH of 8.5); followed by a first elution with 20 mM Tris, 290 mM NaCl, and Pluronic F-68 (mixture pH of pH 8.5); and then a second elution with 20 mM Tris, 305 mM NaCl, and Pluronic F-68 (mixture pH of pH 8.5). The CEX elution pool was neutralized with acetic acid to a mixture pH 7.0. The AEX elution pool was then processed through 0.2 μm Filtration using an EMD Millipore Express SHC XL150 filter, resulting in a CEX pool as an AEX pool equivalent.

### Example 9. Downstream—TFF Filtration

A neutralized AEX pool from Example 8 was provided. The neutralized AEX pool was processed through Tangential Flow Filtration (TFF) using a Spectrum mPES Hollow Fiber TFF system. The TFF system was first rinsed with WFI water, then sanitized with 0.1 M NaOH, then equilibrated with an AEX Elution Buffer (pH 8.0) comprising 20 mM Tris, 220 mM sodium chloride, and Pluronic F-68, with equilibration continuing until both permeate and retentate effluents are at pH 8.0. The AEX pool was processed through Pre-TFF Nanofiltration using an Asahi Kasei Planova 35N filter to produce a TFF Load pool. The TFF Load pool was processed through a first diafiltration (DF) step using a first diafiltration buffer (high salt, low sucrose) which includes 10 mM sodium phosphate, 1.5 mM potassium phosphate, 220 mM sodium chloride, 5% w/v Sucrose, and Pluronic F-68 (buffer pH of 7.5). Diafiltration of the product pool was followed by concentration through ultrafiltration to a target concentration of 5.0-9.0×10¹³ VG/mL (confirmed by qPCR or ddPCR), and then a second diafiltration step using a final formulation buffer (low salt, high sucrose) which includes 10 mM sodium phosphate, 1.5 mM potassium phosphate, sodium chloride, 7% w/v Sucrose, and Pluronic F-68 (buffer pH of 7.5). Retentate comprising the product and final formulation buffer was collected into a Final TFF Pool. Viral titer of the Final TFF Pool was analyzed overnight using qPCR or ddPCR.

The TFF system was subjected to a Recovery Flush using final formulation buffer (low salt, high sucrose) which includes 10 mM sodium phosphate, 1.5 mM potassium phosphate, 100 mM sodium chloride, 7% w/v Sucrose, and Pluronic F-68 (buffer pH of 7.5). The Final TFF Recovery Flush is collected separately from the Final TFF Pool. Viral titer of the Final TFF Recovery Flush was analyzed overnight using qPCR or ddPCR. Final TFF Recovery Flush was added to Final TFF Pool to provide VRF Load Pool with viral concentration of 2.0-5.0×10¹³ VG/mL. Additional final formulation buffer (low salt, high sucrose) was added as necessary to achieve target viral concentration for VRF Load Pool.

In one alternative, the neutralized AEX pool was processed through TFF using a Millipore Pellicon-3 Ultracel PLCTK system. The TFF system was equilibrated with 20 mM Tris, 290 mM sodium chloride, and Pluronic F-68, with equilibration continuing until both permeate and retentate effluents are at pH 7.0. The TFF Load pool was diluted with 20 mM Tris, 290 mM sodium chloride, and 0.001% (w/v) Pluronic F-68 to a viral concentration of 1.0-5.0×10¹² VG/mL. The TFF Load pool was not processed through Pre-TFF Nanofiltration but was instead processed directly into a diafiltration step using a diafiltration buffer which includes 10 mM Sodium Phosphate, 180 mM Sodium Chloride, and Pluronic F-68 (mixture pH of 7.3). The pool was then concentrated through ultrafiltration to a target concentration of 2.5-7.0×10¹² VG/mL. Retentate comprising the product and formulation buffer was collected into a Final TFF Pool. The process did not include a second diafiltration step or a Recovery Flush.

In another alternative, the neutralized AEX pool was processed through TFF using a Millipore Ultracel PLCTK system with Pellicon-3 cassette. The TFF system was first rinsed with WFI water, then sanitized with 0.25 M NaOH, then equilibrated with an equilibration buffer (pH 8.5) comprising 40 mM Tris, 170 mM sodium chloride, and Pluronic F-68, with equilibration continuing until both permeate and retentate effluents are at pH 8.5. The TFF Load pool was not processed through Pre-TFF Nanofiltration or a first diafiltration step, but was instead concentrated through ultrafiltration to a target concentration of 2.5-7.0×10¹² VG/mL (confirmed by qPCR or ddPCR), and then a diafiltration step using a diafiltration buffer which includes 10 mM Sodium Phosphate, 180 mM Sodium Chloride, and Pluronic F-68 (mixture pH of 7.5). The TFF system was subjected to a Recovery Flush using the same diafiltration buffer. The Final TFF Recovery Flush is collected separately from the Final TFF Pool, and each pool is processed separately through 0.2 μm Filtration using an EMD Millipore Express SHCXL150 filter. Filtered TFF Recovery Flush was added to Filtered TFF Pool, and then diluted as needed with diafiltration buffer, to provide VRF Load Pool with viral concentration of 2.5-7.0×10¹² VG/mL.

In another alternative, the TFF system was equilibrated with a mixture of 10 mM sodium phosphate, 2 mM potassium phosphate, 2.7 mM potassium chloride, 192 mM sodium chloride, and Pluronic F-68 (mixture pH of 7.5). The TFF Load pool was not processed through Pre-TFF Nanofiltration or a first diafiltration step, but was instead concentrated through ultrafiltration to a target concentration of 1.5-5.0×10¹³ VG/mL (confirmed by qPCR), and then a diafiltration step using a diafiltration buffer which includes 10 mM Sodium Phosphate, 2 mM Potassium Phosphate, 2.7 mM Potassium Chloride, 192 mM Sodium Chloride, and Pluronic F-68 (mixture pH of 7.5). The TFF system was subjected to a Recovery Flush using a buffer which includes 10 mM Sodium Phosphate, 2 mM Potassium Phosphate. 2.7 mM Potassium Chloride, 192 mM Sodium Chloride, and Pluronic F-68. Final TFF Recovery Flush was added to Final TFF Pool to provide VRF Load Pool with viral concentration of 1.5-5.0×10¹³ VG/mL.

### Example 10. Improvement of Tangential Flow Filtration System—Membrane Constructs

Studies were conducted to improve tangential flow filtration system for processing large volumes of AAV-containing formulations. Multiple membrane constructs were studied to identify specific Tangential Flow Filtration (TFF) system parameters and membrane characteristics which could provide unexpected and improved filtration throughputs, mass balance, product step yields and product recovery yields. Membrane constructs were grouped and evaluated in view of several characteristics, including the following: (i) membrane construction (hollow fiber (HF), flat sheet (FS)): (ii) pore size (100 KDa, 50 KDa, 30KDa); and (iii) membrane chemistry (modified polyethersulfone (mPES), regenerated cellulose (RC)). Membrane constructs were evaluated under various TFF processing conditions, including variations in load volume, load titer, pool volume, pool titer, flush volume and flush titer. Results for product step yield, product retention yield and membrane mass balance were measures and calculated.

The results of these studies are summarized below in Table 12 (Volumes are in milliliters; Titers are in ×10¹² vg/mL).

TABLE 12
TFF Membrane Construct Study

| | | | | | | | | Step | Mass |
|---|---|---|---|---|---|---|---|---|---|
| | Average | Load | Load | Pool | Pool | Flush | Flush | Yield | Balance |
| Membrane | Recovery (%) | Volume | Titer | Volume | Titer | Volume | Titer | (%) | ( %) |
| Spectrum | 15 (N = 6) | 200 | 1.04 | 23 | 1.33 | 15 | 1.65 | 14 | 29 |
| 100 kd | | 200 | 1.14 | 24 | 2.25 | 15 | 1.16 | 23 | 33 |
| HF | | 250 | 0.90 | 23 | 2.01 | 13 | 0.60 | 21 | 26 |
| mPES | | 60 | 0.83 | 10 | 1.10 | 5 | 1.41 | 22 | 39 |
| | | 80 | 0.96 | 10 | 0.48 | 4.5 | 0.84 | 6 | 22 |
| | | 80 | 0.95 | 7 | 0.46 | 6.5 | 0.15 | 4 | 17 |
| Spectrum | 57 (N = 1) | 145 | 1.33 | 21.5 | 5.09 | 15 | 0.98 | 57 | 64 |
| 50 kd | | | | | | | | | |
| HF | | | | | | | | | |
| mPES | | | | | | | | | |
| Biomax | 48 (N = 2) | 200 | 0.98 | 25 | 4.09 | 20 | 0.43 | 52 | 62 |
| FS | | 100 | 0.97 | 17 | 2.51 | 16 | 0.43 | 44 | 51 |
| mPES | | | | | | | | | |
| Ultracell | 70 (N = 4) | 264 | 0.90 | 38 | 4.82 | 30 | 0.63 | 77 | 89 |
| FS | | 168 | 1.5 | 25 | 7.43 | 18.5 | 1.09 | 74 | 82 |
| RC | | 260 | 1.07 | 37 | 5.06 | 13 | 1.12 | 67 | 61 |
| | | 264 | 1.24 | 29 | 6.87 | 12.5 | 2.21 | 61 | 69 |
| Sartorius | 61 (N = 2) | 500 | 1.19 | 65 | 6.02 | 26 | 1.88 | 66 | 56 |
| FS | | 500 | 1.56 | 58 | 7.50 | 22.5 | 4.37 | 56 | 68 |
| RC | | | | | | | | | |

### Example 11. Improvement of Tangential Flow Filtration Process

Studies were conducted to improve the tangential flow filtration process for processing large volumes of AAV-containing formulations. The parameters and results of

TABLE 13
TFF Process Improvement

| | | | | HF | | VRF | | | |
|---|---|---|---|---|---|---|---|---|---|
| | | | | Diameter | 50% | (Pre- | | Final | |
| Run | Shear | TMP | Format | (mm) | Sucrose | TFF) | DF1 Buffer | Formulation | Brief Result |
| 1 | Low | High | DF/UF | 0.5 | | | N/A | PBS | Run Failed |
| | | | | | | | | | during DF |
| 2 | Low | Low | DF/UF | 0.5 | | | N/A | PBS | Run Failed |
| | | | | | | | | | during DF |
| 3 | Low | Low | DF/UF | 1.0 | | | N/A | PBS | Run Failed |
| | | | | | | | | | during DF |
| 4 | Low | Low | DF/UF | 0.5 | X | | N/A | PBS | Run Failed |
| | | | | | | | | | during DF |
| 5 | Low | Low | DF/UF | 0.5 | X | X | N/A | PBS | Success by |
| | | | | | | | | | DSL |
| 6 | Low | Low | DF/UF | 1 | X | X | N/A | PBS | Low |
| | | | | | | | | | turbidity, |
| | | | | | | | | | long |
| | | | | | | | | | processing |
| | | | | | | | | | time |
| 7 | Low | Low | UF/DF | 0.5 | X | X | N/A | PBS | Run Failed |
| | | | | | | | | | during DF |
| 8 | Low | Low | DF/UF | 0.5 | X | X | N/A | VYFORM1 | Run Failed |
| | | | | | | | | | during UF |
| 9 | Low | Low | DF/UF/ | 0.5 | X | X | VYFORM1 | VYFORM1 | Success by |
| | | | DF | | | | 105 mM NaCl | | DLS |
| | | | | | | | 5% (w/v) | | |
| | | | | | | | Sucrose | | |
| 10 | Low | Low | DF/UF/ | 0.5 | X | X | VYFORM1 | VYFORM1 | Run Failed |
| | | | DF | | | | 95 mM NaCl | | during DF1 |
| | | | | | | | 5% (w/v) | | |
| | | | | | | | Sucrose | | |
| 11 | Low | Low | DF/UF/ | 0.5 | X | X | VYFORM1 | VYFORM1 | Success by |
| | | | DF | | | | 105 mM NaCl | | DLS |
| | | | | | | | 5% (w/v) | | |
| | | | | | | | Sucrose | | |
| 12 | Low | Low | DF/UF/ | 0 5 | X | X | VYFORM1 | VYFORM1 | Success by |
| | | | DF | | | | 220 mM NaCl | | DLS |
| | | | | | | | 5% (w/v) | | |
| | | | | | | | Sucrose | | |
| 14 | Low | Low | DF/UF/ | 0.5 | X | X | VYFORM1 | VYFORM1 | Success by |
| | | | DF | | | | 220 mM NaCl | | DLS |
| | | | | | | | 5% (w/v) | | |
| | | | | | | | Sucrose | | |

### Example 12. Formulation Optimization for rAAV Particle Formulations

Initial formulation screening identified a Phosphate/Sucrose/Sodium Chloride formulation (2.7 mM Sodium Phosphate (dibasic). 1.5 mM Potassium Phosphate (mono). 155 mM Sodium Chloride, and 5% (w/v) Sucrose at pH 7.2, 450 mOsm/kg) as an acceptably stable formulation for therapeutic rAAV particles. High salt formulations were also identified as stabilizing.

The formulation was further optimized for excipients, Sodium/Potassium ratios, pH, and osmolality while adjusting for factors suitable for CNS administration. Three solutions that may be used to formulate the therapeutic rAAV particles are presented in Table 14.

TABLE 14
Formulations for therapeutic rAAV particles

| Formulation 1 | Formulation 2 | Formulation 3 |
|---|---|---|
| (VYFORM1 + Pluronic ® F-68) | (VYFORM19 + Pluronic ® F-68) | (VYFORM420 + Pluronic ® F-68) |
| 10 mM Sodium Phosphate | 10 mM Tris Base | 10 mM Tris Base |
| 1.5 mM Potassium Phosphate | 6.3 mM HCl | 9 mM HCl |
| 95 mM Sodium Chloride | 1.5 mM Potassium Chloride | 1.5 mM Potassium Chloride |
| 7% (w/v) Sucrose | 100 mM Sodium Chloride | 100 mM Sodium Chloride |
| 0.001% (w/v) Pluronic ® F-68 | 7% (w/v) Sucrose | 7% (w/v) Sucrose |
| pH 7.4 ± 0.2 at 5° C. | 0.001% (v/v) Pluronic ® F-68 | 0.001% (v/v) Pluronic ® F-68 |
| | pH 8.0 ± 0.2 at 5° C. | DH 7.5 ± 0.2 at 5° C. |

The concentration of the AAV1-HD vector to be formulated in the above identified solutions is about 2.7×10¹³ vg/mL, but the concentration may be increased up to 5×10¹³ vg/ml. High concentration AAV-HD vectors were shown to be difficult to stabilize in the absence of aggregation. Analysis of a formulation screen indicated that an increase in sucrose level generally improves vector stability and prevents aggregation. Sucrose levels from about 5% to 9% provided good stability for the AAV-HD vector, with the optimal concentration at about 7% for the tested vector and desired formulation concentration. The level of sucrose use may be limited by physiological osmolality. Furthermore, higher osmolality and/or more sodium chloride were shown to be favorable for vector stability.

### Example 13. Formulation Optimization Studies

Formulation studies were designed to optimize the formulation components and ratios for optimal stability of the formulation and vector. All tests were run at 5° C. and the samples were maintained at 5° C. during the study.

### Screen I—Buffer Swap

The first set of studies compared the buffer of different formulations. There was a reduction of volume of the formulation prior to dilution with the preferred buffer. Then the formulations were then concentrated to 3.4×10¹³ vg/ml. All formulations aggregated and it was determined that a sugar was needed during concentration to reduce aggregation.

### Screen II—Alternative Buffers and Addition of Sugars

The formulations in this study were dialyzed into the desired buffers and then concentrated to 3×10¹³ vg/ml, 4×10¹³ vg/ml or 5.65×10¹². The formulations tested were (1) VYFORM2 with 0.001% Pluronic® F-68, (2) VYFORM9 with 0.001% Pluronic® F-68, (3) VYFORM10 with 0.001% Pluronic® F-68, (4) VYFORM11 with 0.001% Pluronic® F-68, (5) VYFORM23 with 0.001% Pluronic® F-68, and (6) VYFORM12 with 0.001% Pluronic® F-68.

From the screen, the top alternative buffers were Tris and Histidine and they were used in the formulations in combination with Sucrose and Sodium Chloride. The titer results mostly matched the aggregation trends that were seen for the formulations. Higher aggregation resulted in lower titers.

### Screen III—Sugar Levels

The formulations were dialyzed into the desired buffers and then concentrated to 4×10¹³ vg/ml. The formulations tested were (1) VYFORM7 with 0.001% Pluronic® F-68, (2) VYFORM8 with 0.001% Pluronic® F-68, (3) VYFORM21 with 0.001% Pluronic® F-68, (4) VYFORM22 with 0.001% Pluronic® F-68, (5) VYFORM24 with 0.001% Pluronic® F-68, (6) VYFORM28 with 0.001% Pluronic® F-68, and (7) VYFORM25 with 0.001% Pluronic® F-68.

From this study it was determined that a sugar (e.g., sucrose) was needed with a phosphate buffer for stability of the formulation. The titer results mostly matched the aggregation trends that were seen for the formulations. Higher aggregation resulted in lower titers.

### Screen IV—pH Range on AAV Stability

Formulations of different pH were compared for the stability of AAV a concentration of 5.65×10¹² and 4×10¹³ vg/ml. The formulations tested were (1) VYFORM12 with 0.001% Pluronic® F-68 at a pH of 7, (2) VYFORM26 with 0.001% Pluronic® F-68 at a pH of 7.8, (3) VYFORM26 with 0.001% Pluronic® F-68 at a pH of 6, and (4) VYFORM26 with 0.001% Pluronic® F-68 at a pH of 8.5. The pH was found to have no immediate impact on stability.

### Screen V—Optimization of Formulation Component Ratios

Different component ratios and amounts were evaluated in order to optimize the formulation. The formulations tested that had a pH of 6.9 at 5° C. and 25° C. were (1) VYFORM3 with 0.001% Pluronic® F-68 at osmolality of 428 mOsm/kg, (2) VYFORM4 with 0.001% Pluronic® F-68 at osmolality of 402 mOsm/kg, (3) VYFORM5 with 0.001% Pluronic® F-68 at osmolality of 425 mOsm/kg, and (4) VYFORM6 with 0.001% Pluronic® F-68 at osmolality of 402 mOsm/kg. The formulations tested that had a pH of 7.5 at 5° C. and a pH of 7.4 at 25° C. were (5) VYFORM13 with 0.001% Pluronic® F-68 at osmolality of 424 mOsm/kg, (6) VYFORM14 with 0.001% Pluronic® F-68 at osmolality of 404 mOsm/kg, (7) VYFORM15 with 0.001% Pluronic® F-68 at osmolality of 432 mOsm/kg, (8) VYFORM16 with 0.001%4Pluronic. F-68 at osmolality of 413 mOsm/kg, (9) VYFORM1 with 0.001% Pluronic® F-68 at osmolality of 436 mOsm/kg, and (10) VYFORM8 with 0.001% Pluronic® F-68 at osmolality of 410 mOsm/kg. There were many formulations which were found to have the level of stability needed to continue studies.

### Screen VI—Stability

Provided in Table 15 is a summary of the stability of the formulations tested in Screen II-V. In Table 15, “NT” means not tested, and “−” means less than 85% monomers were seen, “+” means 85-90% monomers were seen, and “++” means 90-100% monomers were seen.

TABLE 15
Formulation Stability Results

| | | TARGET Approx | Time (days) | | | | | | | |
|---|---|---|---|---|---|---|---|---|---|---|
| | Formulation | Conc (vg/ml) | 0 | 1 | 3 | 7 | 11 | 14 | 21 | 28 |
| Screen II | VYFORM2 | 5.65E+12 | + | NT | NT | NT | NT | NT | NT | NT |
| | | 3.00E+13 | ++ | NT | ++ | ++ | + | − | − | − |
| | | 4.00E+13 | ++ | NT | + | − | + | − | + | − |
| | VYFORM9 | 5.65E+12 | + | NT | NT | NT | NT | NT | NT | NT |
| | | 3.00E+13 | − | NT | − | − | − | − | − | − |
| | | 4.00E+13 | − | NT | − | − | − | − | − | − |
| | VYFORM10 | 5.65E+12 | + | NT | NT | NT | NT | NT | NT | NT |
| | | 3.00E+13 | − | NT | − | − | − | − | − | − |
| | | 4.00E+13 | − | NT | − | − | − | − | − | − |
| | VYFORM11 | 5.65E+12 | + | NT | NT | NT | NT | NT | NT | NT |
| | | 3.00E+13 | − | NT | ++ | − | − | − | − | − |
| | | 4.00E+13 | − | NT | − | − | − | − | − | − |
| | VYFORM23 | 5.65E+12 | + | NT | NT | NT | NT | NT | NT | NT |
| | | 3.00E+13 | ++ | NT | ++ | − | ++ | ++ | + | + |
| | | 4.00E+13 | ++ | NT | ++ | + | − | − | − | − |
| | VYFORM12 | 5.65E+12 | | NT | NT | NT | NT | NT | NT | NT |
| | | 3.00E+13 | + | NT | + | ++ | ++ | ++ | ++ | ++ |
| | | 4.00E+13 | ++ | NT | ++ | ++ | ++ | ++ | ++ | − |
| Screen III | VYFORM7 | 5.65E+12 | + | ++ | NT | ++ | ++ | ++ | ++ | ++ |
| | | 4.00E+13 | ++ | ++ | ++ | ++ | ++ | ++ | ++ | ++ |
| | VYFORM8 | 5.65E+12 | + | + | NT | ++ | ++ | − | ++ | ++ |
| | | 4.00E+13 | ++ | + | ++ | ++ | − | ++ | ++ | − |
| | VYFORM21 | 5.65E+12 | + | + | NT | + | − | − | ++ | ++ |
| | | 4.00E+13 | + | − | ++ | + | ++ | ++ | ++ | ++ |
| | VYFORM22 | 5.65E+12 | + | + | NT | ++ | + | ++ | − | + |
| | | 4.00E+13 | ++ | ++ | ++ | ++ | − | − | ++ | ++ |
| | VYFORM24 | 5.65E+12 | + | + | NT | ++ | ++ | ++ | ++ | |
| | | 4.00E+13 | ++ | − | ++ | ++ | ++ | ++ | ++ | + |
| | VYFORM28 | 5.65E+12 | ++ | ++ | NT | ++ | ++ | ++ | − | ++ |
| | | 4.00E+13 | − | − | − | − | − | − | − | − |
| | VYFORM25 | 5.65E+12 | ++ | + | NT | + | ++ | − | ++ | ++ |
| | | 4.00E+13 | ++ | + | + | + | ++ | ++ | + | ++ |
| Screen IV | VYFORM12 (pH 7) | 5.65E+12 | ++ | NT | ++ | ++ | NT | ++ | ++ | ++ |
| | | 4.00E+13 | ++ | NT | ++ | − | NT | ++ | − | − |
| | VYFORM26 (pH 7.8) | 5.65E+12 | ++ | NT | ++ | − | NT | ++ | ++ | ++ |
| | | 4.00E+13 | ++ | NT | ++ | ++ | NT | − | − | − |
| | VYFROM26 (pH 6) | 5.65E+12 | ++ | NT | + | − | NT | + | − | + |
| | | 4.00E+13 | ++ | NT | ++ | ++ | NT | − | − | − |
| | VYFROM26 (pH 8.5) | 5.65E+12 | + | NT | ++ | + | NT | − | ++ | ++ |
| | | 4.00E+13 | + | NT | ++ | + | NT | − | − | − |
| Screen V | VYFORM3 (428 | 5.65E+12 | ++ | ++ | ++ | ++ | NT | ++ | ++ | ++ |
| | mOsm/kg) | 4.00E+13 | ++ | ++ | ++ | ++ | NT | ++ | + | − |
| | VYFORM4 (402 | 5.65E+12 | ++ | ++ | + | ++ | NT | ++ | + | + |
| | mOsm/kg) | 4.00E+13 | + | ++ | ++ | ++ | NT | − | − | + |
| | VYFORM5 (425 | 5.65E+12 | + | ++ | ++ | + | NT | ++ | + | + |
| | mOsm/kg) | 4.00E+13 | − | ++ | ++ | ++ | NT | − | − | + |
| | VYFORM6 (402 | 5.65E+12 | − | − | + | ++ | NT | ++ | + | + |
| | mOsm/kg) | 4.00E+13 | + | ++ | + | − | NT | ++ | + | + |
| | VYFORM13 (424 | 5.65E+12 | ++ | ++ | ++ | ++ | NT | ++ | − | ++ |
| | mOsm/kg) | 4.00E+13 | ++ | ++ | ++ | ++ | NT | ++ | − | − |
| | VYFORM14 (404 | 5.65E+12 | ++ | − | ++ | − | NT | ++ | ++ | + |
| | mOsm/kg) | 4.00E+13 | ++ | − | − | + | NT | − | + | − |
| | VYFORM15 (432 | 5.65E+12 | ++ | ++ | ++ | ++ | NT | ++ | − | ++ |
| | mOsm/kg) | 4.00E+13 | − | ++ | ++ | ++ | NT | − | ++ | ++ |
| | VYFORMI16 (413 | 5.65E+12 | ++ | − | − | ++ | NT | − | + | − |
| | mOsm/kg) | 4.00E+13 | ++ | ++ | ++ | ++ | NT | ++ | ++ | ++ |
| | VYFORM17 (436 | 5.65E+12 | ++ | ++ | − | ++ | NT | ++ | ++ | − |
| | mOsm/kg) | 4.00E+13 | ++ | ++ | ++ | ++ | NT | ++ | ++ | + |
| | VYFORM18 (410 | 5.65E+12 | ++ | ++ | + | ++ | NT | − | ++ | ++ |
| | mOsm/kg) | 4.00E+13 | − | ++ | ++ | ++ | NT | ++ | ++ | − |

Formulations which had high concentrations of AAV were found to be difficult to stabilize. The stability of the formulations tended to increase with the level of sucrose, however the level of stability appeared to level off after 7% sucrose and the formulations with 9% sucrose did not confer any additional stability. Histidine buffered sucrose formulation were found to be highly stable as well. Additionally, formulations with higher osmolality and higher concentrations of sodium chloride were found to provide more stable formulations, increased vector stability, and better formulations for CNS delivery.

### Example 14. Long-term Storage Formulation Stability Study

Formulation 1 (Example 12) was studied for long-term storage stability under various temperature and agitation conditions. An initial sample of AAV particles (AAV1 capsid) in Formulation 1 was provided, having the following properties: Titer—2.77×10¹³ vg/ml; Average Particle Radius (DLS)—16.5 nm; Monomeric Purity (DLS)—100%, Osmolality—462 mOsm/kg; pH—7.36, Relative Potency—82.1.

Certain samples were stored at temperatures ranging from −80° C. to 40° C.; certain samples were subjected to multiple freeze-thaw cycles at 5° C. and 37′° C. and certain samples were exposed to agitation over a5-hour period.

Results from this Long-term Storage Formulation Stability study are summarized below in Table 16.

TABLE 16
Formulation Stability Results

| Storage | Sample | ddPCR Titer | Avg. Radius | Monomeric | Relative |
|---|---|---|---|---|---|
| Temperature | Timepoint (days) | (vg/ml, ×10¹³) | (nm) | Purity (%) | Potency |
| −80° C. | 28 | 2.65 | 15.2 | 100 | 80.2 |
| | 90 | 2.42 | 15.4 | 100 | 106.4 |
| | 182 | 3.12 | 15 | 100 | 93.7 |
| | 274 | 2.73 | 14.8 | 100 | 75.5 |
| −40° C. | 28 | 2.71 | 4.6 | 100 | 77.6 |
| | 90 | 2.56 | 13.2 | — | 91.4 |
| | 182 | 3.17 | 15.7 | 100 | 96.4 |
| | 274 | 2.82 | 15.2 | 100 | 82.5 |
| −20° C. | 28 | 2.96 | 14.6 | 100 | 69.9 |
| | 90 | 2.11 | 16.2 | 100 | 95.3 |
| | 182 | 2.99 | 15.5 | 100 | 69.8 |
| | 274 | 3.06 | 16.7 | 100 | 68.3 |
| 4° C. | 7 | 3.02 | 15.9 | 100 | 72.9 |
| | 14 | 2.47 | 15.4 | 100 | 94.4 |
| | 28 | 2.9 | 16.5 | 100 | 68.7 |
| | 90 | 2.34 | 13.6 | 94.4 | 90.6 |
| | 182 | 3.27 | 20.9 | 46.7 | 58.2 |
| | 274 | 3.04 | 21.3 | 37.4 | 61.3 |
| 25° C. | 7 | 2.49 | 14.4 | 100 | 72.0 |
| | 14 | 2.85 | 15.4 | 100 | 51.7 |
| | 21 | 2.81 | 16.5 | 100 | 57.5 |
| | 28 | 2.77 | 15.8 | 100 | 52.6 |
| | 90 | 1.38 | 17.5 | 74 | 61.7 |
| 40° C. | 7 | 2.5 | 19.2 | 100 | 35.6 |
| | 14 | 2.3 | 17.3 | 100 | 20.3 |
| | 21 | 2.27 | 18.8 | 95.1 | 9.0 |
| | 28 | 2.22 | 19.7 | 99.3 | 4.2 |
| | 90 | 1.22 | 18.4 | 91.3 | 0 |
| Freeze/Thaw | Freeze/Thaw | ddPCR Titer | Avg. Radius | Monomeric | Relative |
| Temperature | Cycles | (vg/ml, ×10¹³) | (nm) | Purity (%) | Potency |
| F: −80° C. | 1x | 2.61 | 14.5 | 84 | 87.7 |
| T: 5° C. | 3x | 2.52 | 15.1 | 85 | 70.9 |
| | 5x | 2.63 | 16 | 85 | 71.3 |
| | 7x | 2.61 | 15.1 | 86 | 68.8 |
| F: −80° C. | 1x | 2.51 | 15.4 | 84 | 70.7 |
| T: 37° C. | 3x | 2.59 | 16.1 | 85 | 61.5 |
| | 5x | 9.60 | 15.1 | 83 | 67.3 |
| | 7x | 2.55 | 15.8 | 85 | 73 |

| | ddPCR Titer | Avg. Radius | Monomeric | Relative |
|---|---|---|---|---|
| Agitation Timepoint (hours) | (vg/ml, ×10¹³) | (nm) | Purity (%) | Potency |
| 0.5 h | 2.48 | 120.6 | — | 82.9 |
| 1.0 h | 2.29 | 15.3 | 82.8 | 83.8 |
| 3.0 h | 2.63 | 14.3 | 38.4 | 81.3 |
| 5.0 h | 2.49 | 61.6 | — | 88.2 |

Results of the long-term storage formulation stability study showed that Formulation 1 provided the following: (i) consistent AAV titer at storage temperatures of ≤4° C. for up to 274 days (testing limit); (ii) consistently high monomeric purity (i.e. low AAV particle aggregation) at storage temperatures of ≤4° C. for up to 100 days storage, and temperatures of ≤−20° C. for up to 274 days (testing limit); (iii) consistent AAV potency at storage temperatures of ≤4° C. for up to 274 days (testing limit); and (iv) consistent AAV titer, high monomeric purity, and AAV potency through 7 freeze/thaw cycles.

### Example 15. Downstream—Virus Retentive Filtration

A VRF Load Pool from Example 9 was provided. The VRF Load Pool was processed through Virus Retentive Filtration (VRF) using an Asahi Kasei Planova 35N filter which had been processed through a pre-use flush with a formulation buffer of 10 mM sodium phosphate, 1.5 mM potassium phosphate, 100 mM sodium chloride, 7% w/v Sucrose, and Pluronic F-68 (buffer pH of 7.5). The VRF filtration was followed by processing through 0.2 μm Filtration using an EMD Millipore Express SHCXL150 filter, resulting in a VRF pool with a working viral concentration of 2.5-7.0×10¹³ VG/mL.

The VRF pool was then processed through Millipore Final Filtration (FF) using an EMD Millipore Sterile Millipak 0.22 μm to provide a Drug Substance pool with a working viral concentration of 1.5-5.0×10¹³ VG/mL. A portion of the Drug Substance pool was stored for ≤1 month at 2-8° C. in aseptic bioprocess bag closed to atmosphere. A portion of the Drug Substance pool was stored for ≥1 month at ≤−60° C. in aseptic Polypropylene container closed to atmosphere.

In one alternative, the VRF filter and FF filters are both pre-use-flushed with WFI water, followed by a second pre-use-flush with 10 mM sodium phosphate. 180 mM sodium chloride, and Pluronic F68 (mixture pH of 7.3).

In one alternative, the VRF filter is pre-use-flushed with a mixture of 10 mM sodium phosphate, 2 mM Potassium Phosphate, 2.7 mM Potassium Chloride, 192 mM Sodium Chloride, and Pluronic F68 (mixture pH of 7.5).

### Example 16. Downstream—Fill and Finish

A Pooled Drug Substance from Example 15 was provided. The Pooled Drug Substance was transferred to a Biosafety Cabinet (BSC) and filtered through a EMD Millipore Millipak Gamma Gold 0.22 μm filter (dual-in-line sterilizing grade filters). The filtered Drug Substance pool was then aseptically filled into 2 ml Cryovials utilizing a programmable Peristaltic dispensing pump within the BSC. Product vials were stoppered, seal capped, 100% visually inspected and labeled (at 25° C.), and then stored at ≤−65° C.

In one alternative, the Pooled Drug Substance was filtered through a Pall Supor EKV, 0.2 μm sterilizing-grade filter.

### Example 17. Cumulative Viral Clearance Study

Process steps from Example 2 (lysis detergent), Example 7 (Affinity Chromatography), Example 8 (Ion Exchange Chromatography) and Example 15 (Viral Retention Filtration) were studied for their effectiveness at inactivating known viral contaminants within bulk harvest pools of AAV particles produced using Baculovirus-production systems and Sf9 insect cells. Baculovirus (BACV) is a known process contaminant: Vesicular Stomatitis Virus (VSV) is used as a model for known Rhabdoviral cell line contaminants; Human Adenovirus Type 5 (Ad5) is a known process contaminant which can act as a helper virus to facilitate unwanted AAV replication; and Reovirus Type 3 (Reo3) is used as a representative model for known dsRNA viral contaminants.

Results from this viral clearance study are summarized below in Table 17. Values represent Log₁₀ reduction values for viral contaminant (TCID50); “NV” indicates that no value was collected.

TABLE 17
Viral Clearance Study

| System | Run | BACV | VSV | AD5 | REO3 |
|---|---|---|---|---|---|
| Heat inactivation | 1 | No Reduction | No Reduction | NV | NV |
| (37° C.) | 2 | No Reduction | No Reduction | NV | NV |
| Detergent | 1 | >5.24 | >4.45 | NV | NV |
| (Triton X-100) | 1 | >5.06 | >4.37 | NV | NV |
| Affinity Chromatography | 1 | 4.13 | >4.56 | NV | NV |
| (AVB Sepharose HP) | 2 | 4.62 | 5.24 | NV | NV |
| CEX Chromatography | 1 | 1.44 | No Reduction | NV | NV |
| (Poros XS) | 2 | 1.35 | No Reduction | NV | NV |
| AEX Chromatography | 1 | 5.67 | >6.66 | 7.02 | 7.14 |
| (Fractogel TMAE HiCap) | 2 | 5.95 | >6.72 | 6.09 | 6.84 |
| VRF | 1 | >5.08 | >4.65 | NV | NV |
| (Planova 35N) | 2 | >4.74 | >5.00 | NV | NV |

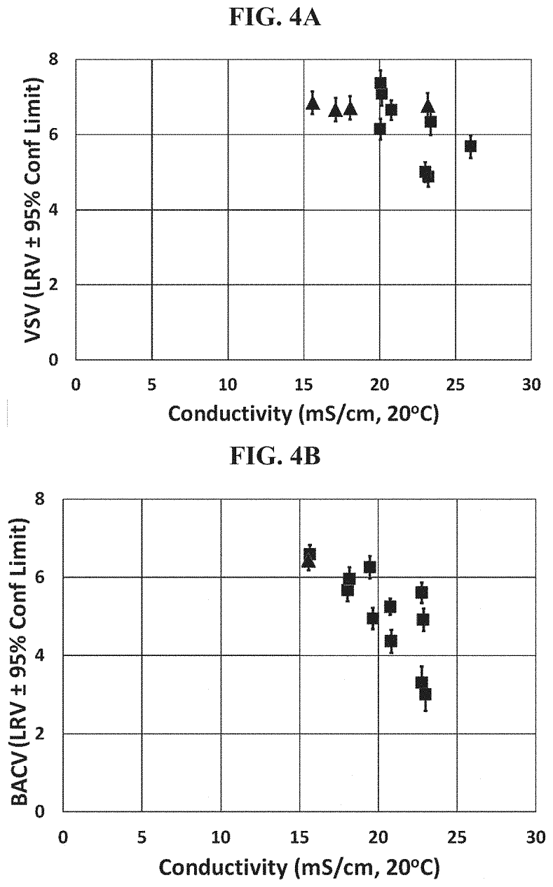

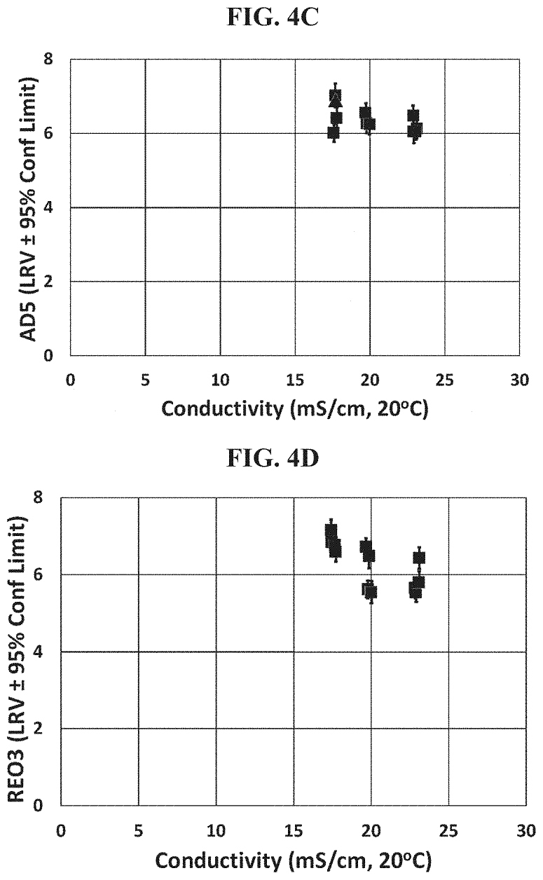

Results show that a combination of the process steps from Example 2 (lysis detergent), Example 7 (Affinity Chromatography), Example 8 (Ion Exchange Chromatography) and Example 15 (Viral Retention Filtration) can provide Log₁₀ viral reduction value of more than 20. The use of flow-through AEX chromatography provides notably robust viral clearance, as seen in Table 17 and FIGS. 4A-4D.

### Example 18. Dose Optimization Study I

i. Study design

The primary objective of this study was to evaluate delivery parameters to optimize distribution of an AAV1 packaged AAV1-miRNA expression vector comprising an ITR-to-ITR sequence. VOYHT1, (hereinafter referred to as AAV1-VOYHT1; SEQ ID NO for VOYHT1: 41) within the striatum, cortex and thalamus of rhesus macaques, and to provide a basis for establishing future dosing parameters and for extrapolation to a clinical dosing paradigm. A secondary objective was to conduct a limited safety and tolerability assessment of delivery parameters.

The rhesus macaque (Macaca mulatta) was selected as the test system due to its established usefulness and acceptance as a model for pharmacological and toxicological studies, especially when using gene therapy delivery to the central nervous system (CNS). The more completely understood mapping of rhesus genome, relative to other non-human primates (NHPs), is particularly relevant for assessment of RNA interference products. The large brain volume and anatomical structure were also important factors taken into consideration when choosing this species to address the study objectives.

This study involved the screening of 34 animals to obtain 18 for dosing and 2 alternates. The 18 animals were assigned to 6 treatment groups as summarized in Table 18. Bilateral intraparenchymal infusion into the putamen and thalamus was chosen to maximize brain distribution via axonal transport to cortical areas. Also, putamen and thalamus were preferred infusion sites because putamen and thalamus in early HD human patients are 4-5 times larger than in rhesus, and severe atrophy of the caudate nucleus would prevent direct infusion into the caudate.

TABLE 18
Study design

| | | Number of | Volume (μL/side) | | Dosing Titer | Total |
|---|---|---|---|---|---|---|
| Group | Description | animals | Putamen | Thalamus | (vg/ml) | dose (vg) |
| Group Al | Low Vol; High Conc. | 3 | 50 | 75 | 2.7e12 | 6.8e11 |
| Gtoup A2 | Mid Vol; High Conc. | 3 | 100 | 150 | 2.7e12 | 1.4e12 |
| Group A3 | High Vol; High Conc. | 3 | 150 | 250 | 2.7e12 | 2.2e12 |
| Group A4 | Mid Vol; Mid Conc.. | 3 | 100 | 150 | 9.0e11 | 4.5e11 |
| Group A5 | Mid Vol; Lower Conc. | 3 | 100 | 150 | 2.7e11 | 1.4e11 |
| Group A6 | Vehicle Control | 3 | Left: 100 | Left: 150 | N/A | N/A |
| | | | Right: 150 | Right: 250 | | |

The calculated human equivalent dose corresponding to each group in Table 18 is presented in Table 19.

TABLE 19
Human equivalent dose

| | | Human Equivalent Dose | | |
|---|---|---|---|---|
| | | | | Total |
| | | Putamen | Thalamus | dose |
| Group | Description | (μL/side) | (μL/side) | (vg) |
| Group A1 | Low Vol; High Conc. | 175 | 450 | 3.4e12 |
| Group A2 | Mid Vol; High Conc. | 350 | 900 | 6.8e12 |
| Group A3 | High Vol; High Conc. | 525 | 1500 | 1.1e13 |
| Group A4 | Mid Vol; Mid Conc. | 350 | 900 | 2.3e12 |
| Group A5 | Mid Vol; Lower Conc. | 350 | 900 | 6.8e11 |
| Group A6 | Vehicle Control | Left: 350 | Left: 900 | N/A |
| | | Right: 525 | Right: 1500 | |

Each animal received bilateral intracranial infusion of the test article containing AAV1-VOYHT1 or a vehicle control into the putamen and thalamus using magnetic resonance imaging (MRI)-guided convection-enhanced delivery (CED). Animals were euthanized 5 weeks (Day 36±3) after dosing, and tissues were collected for post-mortem analysis.

ii. Animal Care and Sample Collection

Thirty-four (N=34) healthy adult male or female rhesus macaques (4-10 years old) were selected for prescreening. Animals weighed 4-10 kg. Animals were acclimated for a minimum of 2 weeks after clearance from Centers for Disease Control and Prevention (CDC) quarantine. Animals had a pre-project blood sample collected for screening of anti-AAV1 neutralizing antibodies (nAb) titers. Eighteen (N=18) animals with anti-AAV1 NAb serum titers ≤1:16 were selected, weighed, and randomized into the study groups for dosing as indicated in Table 18. An additional 2 animals were selected as alternate study animals. Animals were maintained on Harlan 20% Primate Diet with ad libitum access to water. Samples of water were routinely analyzed for specified microorganisms and environmental contaminants. Environmental controls for the animal room were set to maintain 70±6° F., a minimum of 10 air changes/hour, and a 12-hour light/12-hour dark cycle. Cage-side monitoring were performed twice daily and food consumption assessment was performed once daily. Body weight was measured once per week. Animals were housed in individual cages throughout the study.

Blood samples were collected for clinical pathology evaluation and neutralizing antibody (nAb) analysis at Pre-dose (i.e., 7 days prior to the initiation of dosing of the first animal receiving AAV1-VOYHT1 infusion), Day 15±2, and immediately prior to necropsy on Day 36±3. The clinical pathology evaluation included hematology (CBC), serum clinical chemistry (Chem), and coagulation (Coag) analysis. Cerebrospinal fluid (CSF) samples were collected for nAb analysis from the cervical region prior to dosing (Day 1), and immediately prior to necropsy on Day 36±3. Following necropsy, the brain, spinal cord, dorsal root ganglia, and major organs were collected and then fresh frozen or 4% paraformaldehyde (PFA) post-fixed by immersion.

iii. Test Article Preparation and Dosing Procedures

The test article used in the study contained AAV1-VOYHT1 gene transfer vector (2.7e12 vg/mL) formulated in aqueous solution containing 192 mM sodium chloride, 10 mM sodium phosphate, 2 mM potassium phosphate, 2.7 mM potassium chloride, and 0.001% Poloxamer 188 (Pluronic® F-68). The vehicle control contained the formulation buffer only. The samples were stored at −60° C. or below and were thawed to and maintained at 2-8° C. on day of dosing. ProHance® (Bracco Diagnostics, Inc), i.e. gadoteridol, was added at a 1:250 ratio (1 μL of ProHance per 250 μL of test article or control) and carefully mixed by inverting tubes prior to loading into the infusion system. The dosing solution contained the test article or control and a 2 mM concentration of gadoteridol. Dilution of the dosing solutions are summarized in Table 20. “N/A” indicates data not applicable.

TABLE 20
Dilutions of dosing solutions

| | Final vector | Vector | | | Final |
|---|---|---|---|---|---|
| | concentration | stock | Diluent | ProHance | Volume |
| Group | (vg/mL) | (μL) | (μL) | (μL) | (μL) |
| A1, A2, A3 | 2.7e12 | 2250 | 0 | 9 | 2259 |
| A4 | 9.0e11 | 700 | 1392 | 8.4 | 2100 |
| A5 | 2.7e11 | 210 | 1882 | 8.4 | 2100 |
| A6 | N/A | N/A | 15000 | 60 | 15060 |

Immediately prior to surgery, each animal was anesthetized with intramuscular (IM) Ketamine (10 mg/kg) and IM dexmedetomidine (15 μg/kg), weighed, intubated, and maintained on 1-5% Isoflurane. The head was secured onto a stereotaxic frame and the overlying skin prepared for neurosurgical implantation procedures. Using aseptic techniques, the wound site was opened in anatomical layers to expose the skull. A bilateral craniotomy was performed at entry sites located above the frontal and/or parietal lobe on each side. A skull mounted cannula guide ball array was temporarily secured to the skull over each burr hole using titanium screws. Immediately after surgery to implant cannula guides, the animal was transferred to the MRI suite. MR imaging was used to align cannula guides with putamen and thalamus targets ipsilateral to each cannula guide. Test article or control was administered with repeated MR imaging to visually monitor infusions within the brain as specified in Table 18 above. Each animal received up to 2 infusions (sites) of test article or control using convection enhanced delivery (CED) in each putamen and thalamus. An adjustable tip 16G cannula (MRI Interventions Inc.) was guided into each target site through the skull mounted cannula arrays. The cannula was connected to a syringe mounted on a syringe pump (Harvard Apparatus). Dose volumes (50-400 μL per hemisphere) were deposited into each putamen or thalamus using ascending infusion rates (up to 10 L/minute). Serial MRI scans were acquired to monitor infusate distribution within each target site and provide real-time monitoring of the dosing. In some cases, cannula was advanced deeper into the putamen or thalamus during the infusion to maximize infusate distribution within the putamen or thalamus. Immediately after the MRI CED dosing procedure, the animal was transferred back to the operating room, the cannula guide system was explanted, and the wound site was closed in anatomical layers with absorbable vicryl suture and using a simple interrupted suturing pattern. Pre- and post-operative medications included buprenorphine (0.03 mg/kg, IM, b.i.d.), carprofen (2.2 mg/kg SQ, b.i.d.), ketoprofen (2 mg/kg, IM, s.i.d.), and cefazolin (100 mg IV, pre- and post-surgery, followed by 25 mg/kg, IM, b.i.d) or ceftriaxone (50 mg/kg, IM, s.i.d.). Animals were monitored for full recovery from anesthesia and returned to their home cages.

iv. HTT Knockdown and Vector Genome (VG) Measurement in Punches from NHP Striatum, Cortex, and Thalamus Across Different Infusion Volumes

This analysis was designed to evaluate the impact of different infusion volumes on vector distribution and coverage. Selected brain slabs containing the motor and somatosensory cortex and anterior putamen from Groups A1 (low vol), A2 (med vol), A3 (high vol), and A6 (control) were used to collect 2 mm punches. Six cortex, 8 putamen, 2 caudate, and 5 thalamus punches were collected from each side of the brain (42 total per animal), with a total number of 504 punches collected from all four groups. Samples were homogenized in QuantiGene® homogenization buffer and subjected to protease K digest. Cleared cell lysates were generated and processed for both HT mRNA measurement using a branched DNA (bDNA) assay and vector genome (VG) measurement using droplet digital PCR (ddPCR) after an additional DNA purification step (Qiagen, catalog #69506). The bDNA assay was carried out according to the QuantiGene® Plex Assay (ThermoFisher Scientific) protocol using a probe set specifically for rhesus HTT. Cell lysate was assayed in duplicates. HTT mRNA level was normalized to the geometric mean of three rhesus housekeeping genes, i.e., AARS, TBP, and XPNPEP1. Results were calibrated to the normalized mean of the vehicle group and presented as: mean of relative remaining HIT mRNA (%)±stdev. For ddPCR, whole cell DNA was prepared from same tissue homogenate used in the bDNA assay. The level of vector genome detected with probe set, CBA Promoter, was normalized to a Host probe set (RNase P). All samples were blinded during the analysis.

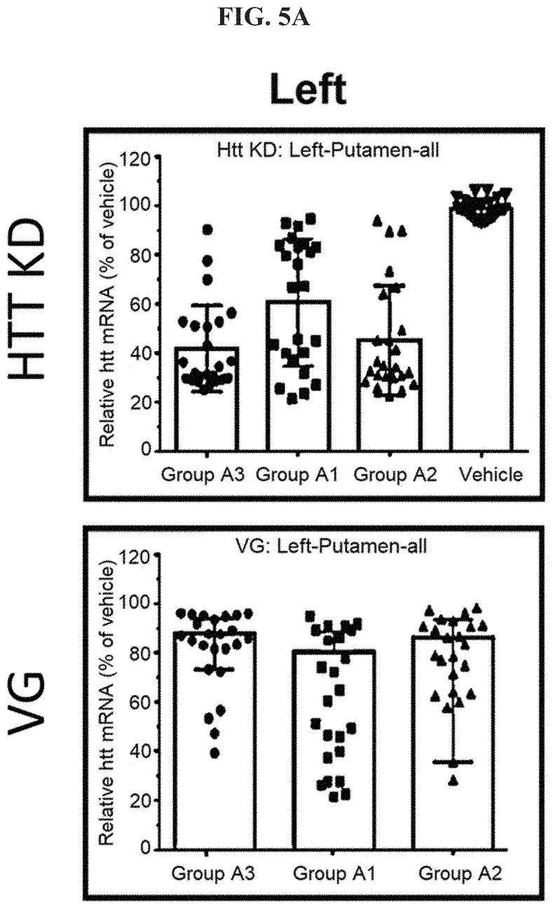

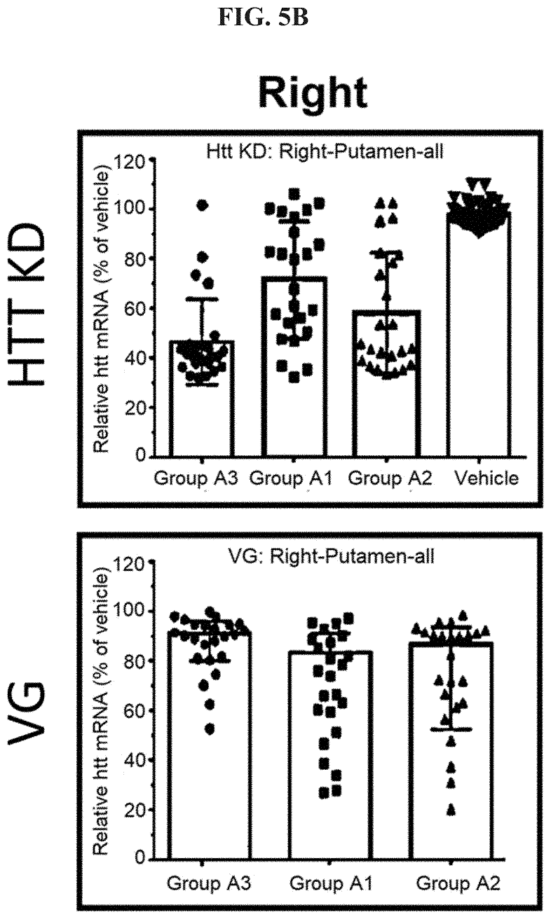

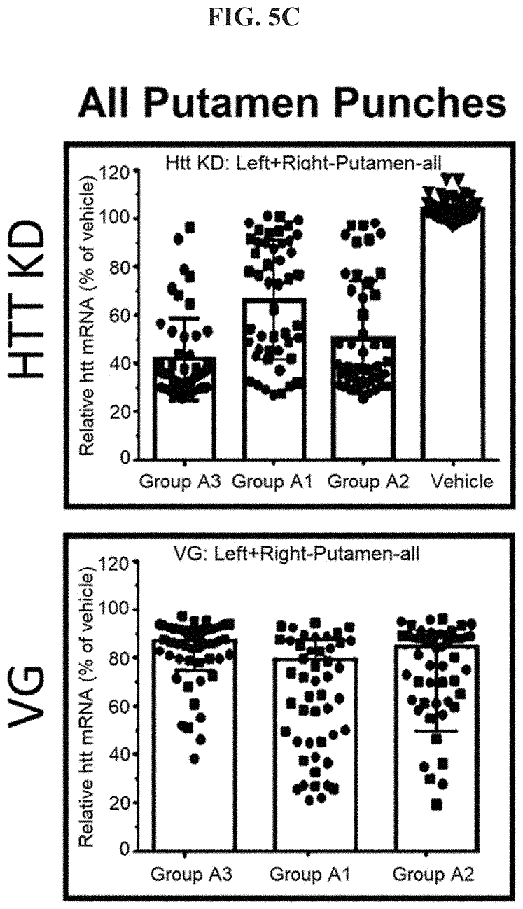

In the putamen, all groups showed HTT mRNA knockdown, with 63%, 48% and 39% HTT mRNA remaining relative to vehicle for Groups A1, A2, and A3, respectively (see FIGS. 5A-5C). Of the 16 punches per animal (total 3 animals per group), an average of 52%, 79%, and 92% of the punches for Group A1, A2 and A3 respectively, reached at least 30% HTT mRNA knockdown. HTT mRNA levels in putamen from each AAV1-VOYHT1-treated group averaged per animal after normalization to the vehicle control group are presented in Table 21.

TABLE 21
HTT mRNA knockdown in putamen punches across
infusion volumes averaged per animal

| | | Number | HTT mRNA |
|---|---|---|---|
| | | of | relative to vehicle |
| Group | Description | animals | (% ± stdev) |
| Group A1 | Low Vol; High Conc. | 3 | 63 ± 12 |
| Group A2 | Mid Vol; High Conc. | 3 | 48 ± 7 |
| Group A3 | High Vol; High Conc. | 3 | 39 ± 8 |
| Group A6 | Vehicle Control | 3 | 100 ± 1 |

When VG copies were analyzed in all putamen punches sampled from each of the three groups, differential VG distributions were observed across different vector infusion volumes. The highest and most stable VG distribution pattern was observed in Group A3, followed by Group A2 and Group A1 (FIGS. 5A-5C). This differential vector distribution pattern was observed in left and right hemispheres. VG levels tracked with putamen HTT knockdown, with Group A3 possessing both the highest VG representation and the greatest HTT mRNA knockdown. For VG levels, the number of VG copies detected in putamen punches from each group averaged per animal is resented in Table 22.

TABLE 22
VG copies in putamen punches across infusion
volumes averaged per animal

| | | Sample | VG copies/cell |
|---|---|---|---|
| Group | Description | size | (mean ± stdev) |
| Group A1 | Low Vol; High Conc. | 3 | 327.2 ± 191.2 |
| Group A2 | Mid Vol; High Conc. | 3 | 527.5 ± 207.0 |
| Group A3 | High Vol; High Conc. | 3 | 710.5 ± 163.8 |
| Group A6 | Vehicle Control | 3 | 0.2 ± 0.2 |

A Grubbs' test (Q=0.1%) was applied for removal of outliers and the VG copies/cell recalculated. Following this post-hoc statistical analysis, VG copies in putamen punches per animal were unchanged for groups A1 and A3, but for Group A2, VG copies/cell was recalculated to 489.7±204.0.

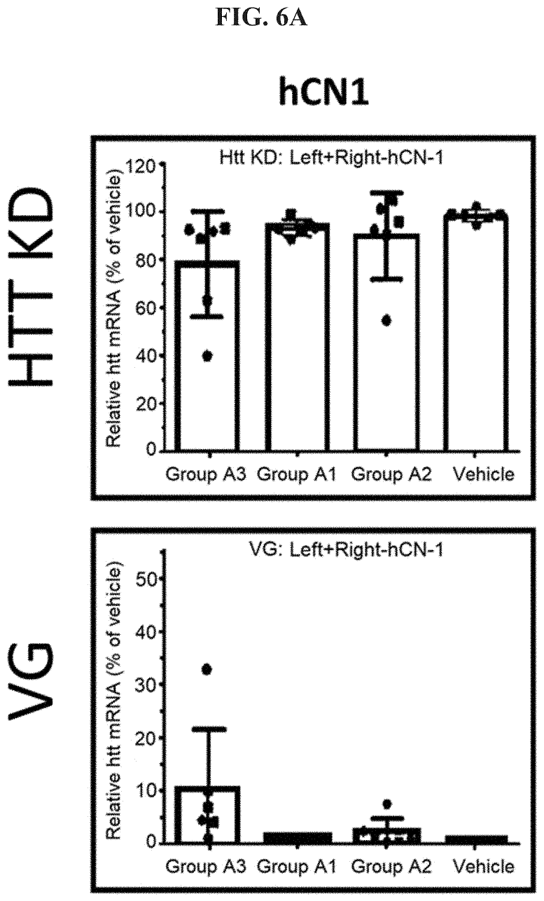

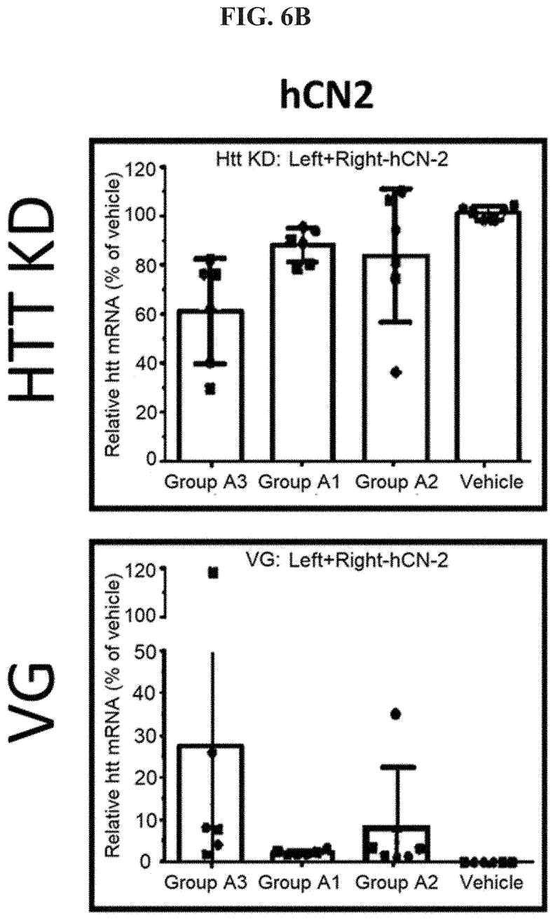

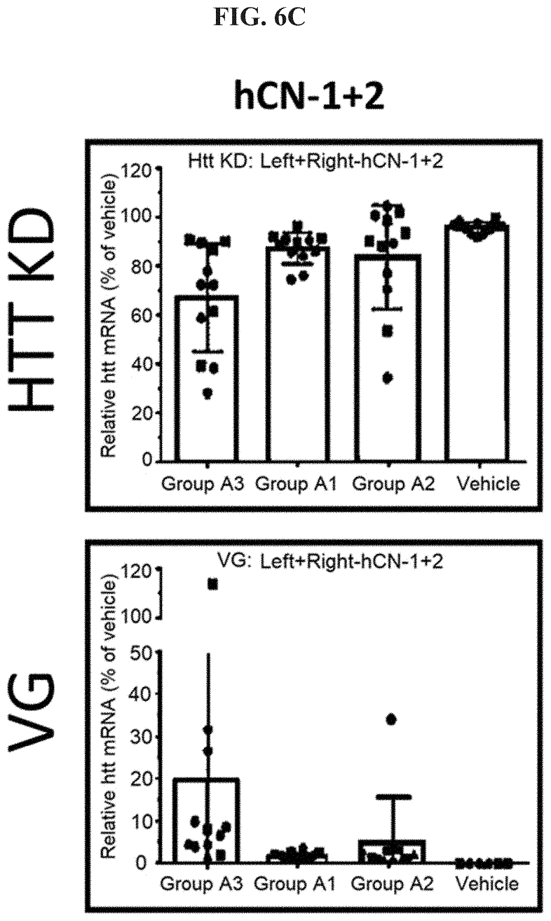

In the caudate, Group A3 showed the greatest HTT mRNA knockdown with 70% HTT mRNA remaining relative to vehicle (see FIGS. 6A-6C). Group A1 and Group A2 showed 91% and 87% HTT mRNA remaining relative to vehicle, respectively. VG levels correlated with HTT mRNA knockdown (see FIGS. 6A-6C). HTT mRNA levels in caudate from each AAV1-VOYHT1-treated group averaged per animal after normalization to the vehicle control group are presented in Table 23.

TABLE 23
HTT mRNA knockdown in caudate punches
across infusion volumes averaged per animal

| | | | HTT mRNA |
|---|---|---|---|
| | | Number | relative to vehicle |
| Group | Description | of animals | (% ± stdev) |
| Group A1 | Low Vol; High Conc. | 3 | 91 ± 2 |
| Group A2 | Mid Vol; High Conc. | 3 | 87 ± 18 |
| Group A3 | High Vol; High Conc. | 3 | 70 ± 23 |
| Group A6 | Vehicle Control | 3 | 100 ± 1 |

When VG copies were analyzed in all caudate punches sampled from each of the three groups, VG levels tracked with HTT mRNA knockdown (see FIGS. 6A-6C). Hence, Group A3 exhibited the highest VG representation and the greatest HTT mRNA knockdown. For VG levels, the number of VG copies detected in caudate punches from each group averaged per animal is presented in Table 24.

TABLE 24
VG copies in caudate punches across infusion volumes averaged per animal

| | | Sample | VG copies/cell |
|---|---|---|---|
| Group | Description | size | (mean ± stdev) |
| Group A1 | Low Vol; High Conc. | 3 | 1.6 ± 0.3 |
| Group A2 | Mid Vol; High Conc. | 3 | 4.9 ± 5.4 |
| Group A3 | High Vol; High Conc. | 3 | 18.8 ± 24.2 |
| Group A6 | Vehicle Control | 3 | 00.0 ± 0.1 |

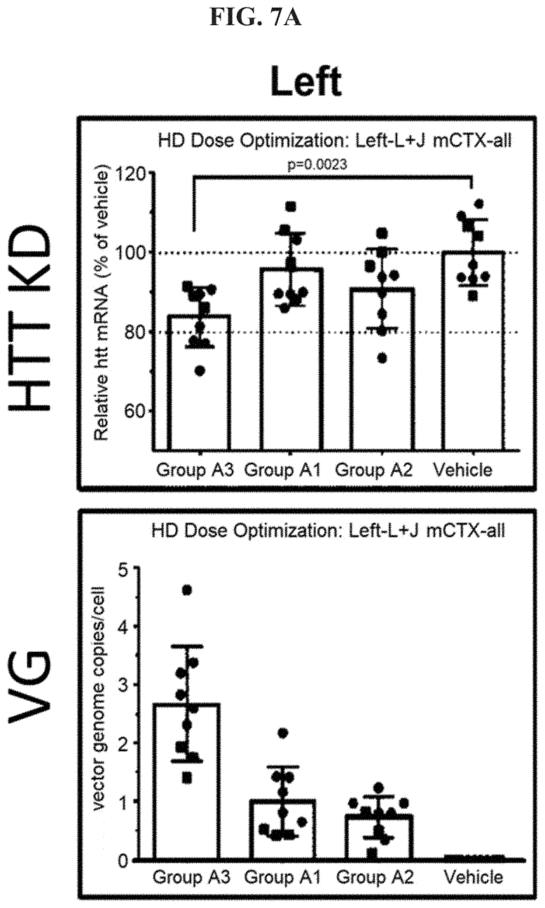

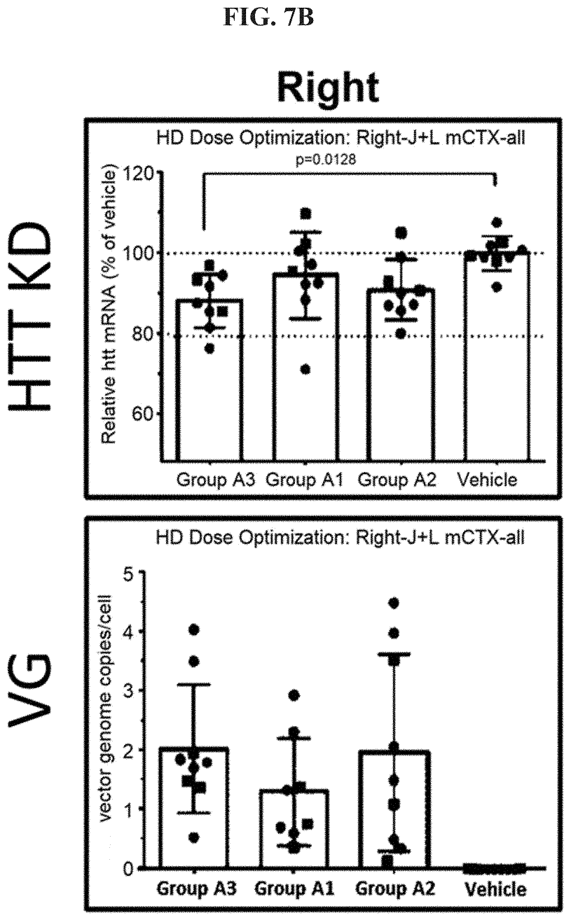

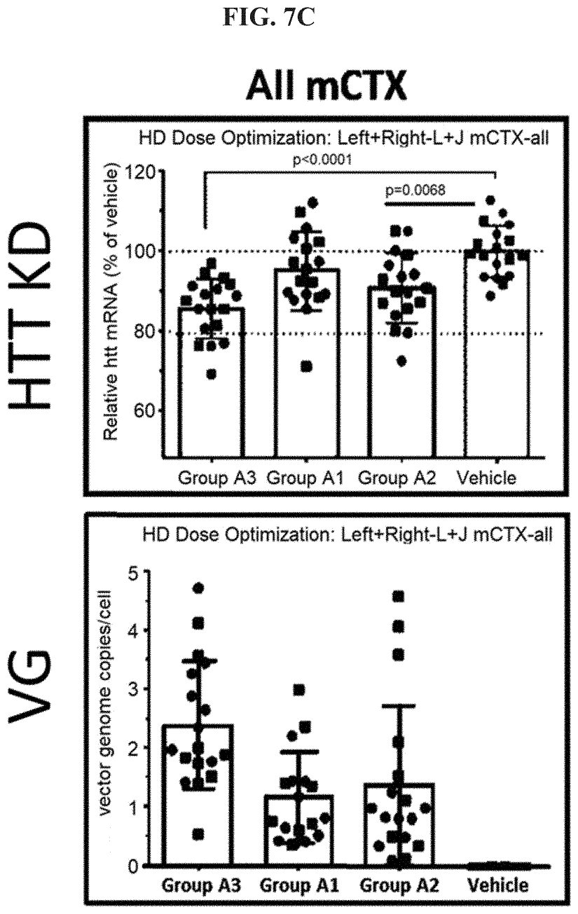

When a Grubbs' test (Q=0.1%) was applied to remove outliers, the average number of VG copies/cell detected in caudate punches remained unchanged for Group A1, but was requantified as 1.8±0.5 and 10.7±10.3 for Groups A2 and A3, respectively. Punches were analyzed from three cortical areas: motor cortex (mCTX), somatosensory cortex (ssCTX), and temporal cortex (tCTX). In the mCTX, significant HTT knockdown was observed for Groups A3 and A2, with greater knockdown in Group A3 than Group A2, resulting in 86% and 91% HTT mRNA remaining relative to vehicle, respectively (see FIGS. 7A-7C). HTT mRNA levels in mCTX from each AAV1-VOYHT1-treated group averaged per animal after normalization to the vehicle control group are presented in Table 25.

TABLE 25
HTT mRNA knockdown in mCTX punches across
infusion volumes averaged per animal

| | | | HTT mRNA |
|---|---|---|---|
| | | Number | relative to vehicle |
| Group | Description | of animals | (% ± stdev) |
| Group A1 | Low Vol; High Conc. | 3 | 95 ± 1 |
| Group A2 | Mid Vol; High Conc. | 3 | 91 ± 3 |
| Group A3 | High Vol; High Conc. | 3 | 86 ± 6 |
| Grou2 A6 | Vehicle Control | 3 | 100 ± 3 |

When VG copies were analyzed in all mCTX punches sampled from each of the three groups, VG levels were lower in mCTX than in the putamen in all groups, with Group A3 showing the highest VG representation (see FIGS. 7A-7C). VG variability was seen between left and right sides of the mCTX. For VG levels, the number of VG copies detected in mCTX punches from each group averaged per animal is presented in Table 26. VG copies were below the quantification limit (BLQ) for Group A6 (vehicle control).

TABLE 26
VG copies in mCTX punches across infusion volumes averaged per animal

| | | Sample | VG copies/cell |
|---|---|---|---|
| Group | Description | size | (mean ± stdev) |
| Group A1 | Low Vol; High Conc. | 3 | 1.36 ± 0.8 |
| Group A2 | Mid Vol; High Conc. | 3 | 1.34 ± 1.03 |
| Group A3 | High Vol; High Conc. | 3 | 2.35 ± 0.3 |
| Group A6 | Vehicle Control | 3 | BLQ |

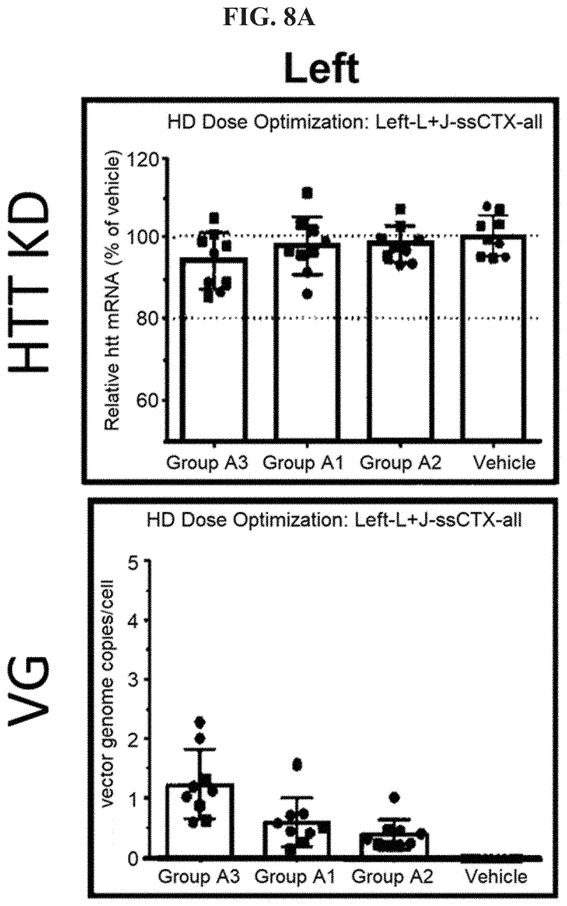

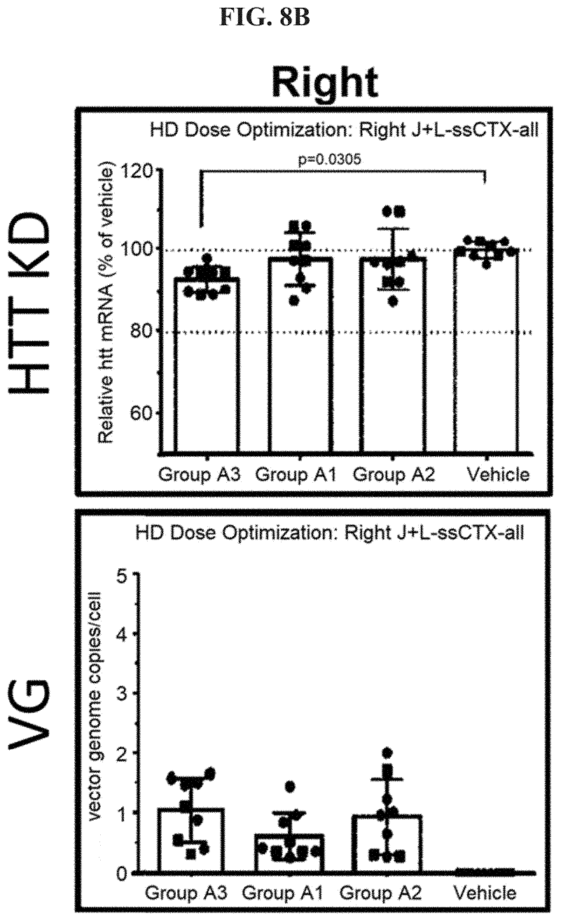

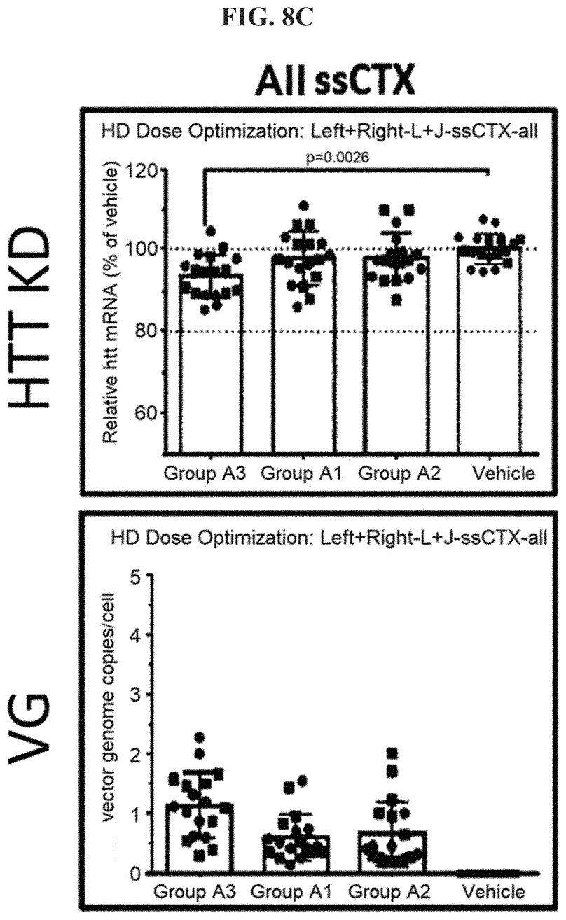

In the ssCTX, HTT knockdown was seen in somatosensory cortex of Group A3 only, where 93% of HIT mRNA remained relative to vehicle (see FIGS. 8A-8C). HTT mRNA levels in ssCTX from each AAV1-VOYHT1-treated group averaged per animal after normalization to the vehicle control group are presented in Table 27.

TABLE 27
HTT mRNA knockdown in ssCTX punches
across infusion volumes averaged per animal

| | | | HTT mRNA |
|---|---|---|---|
| | | Number | relative to vehicle |
| Group | Description | of animals | (% ± stdev) |
| Group A1 | Low Vol; High Conc. | 3 | 96 ± 4 |
| Group A2 | Mid Vol; High Conc. | 3 | 97 ± 3 |
| Group A3 | High Vol; High Conc. | 3 | 94 ± 6 |
| Group A6 | Vehicle Control | 3 | 100 ± 2 |

When VG copies were analyzed in all ssCTX punches sampled from each of the three groups. VG levels were detected at levels lower than observed in mCTX in all groups, and Group A3 had a relatively higher VG representation than Group A1 and Group A2 (see FIG. 8). For VG levels, the average number of VG copies detected in mCTX punches from each group averaged per animal is presented in Table 28. VG copies were below the quantification limit (BLQ) for Group A6 (vehicle control).

TABLE 28
VG copies in ssCTX punches across infusion volumes averaged per animal

| | | Sample | VG copies/cell |
|---|---|---|---|
| Group | Description | size | (mean ± stdev) |
| Group A1 | Low Vol; High Conc. | 3 | 0.61 ± 0.3 |
| Group A2 | Mid Vol; High Conc. | 3 | 0.67 ± 0.3 |
| Group A3 | High Vol; High Conc. | 3 | 1.13 ± 0.3 |
| Group A6 | Vehicle Control | 3 | BLQ |

Combined mCTX and ssCTX samples were also included in cortical punch analyses. When mCTX and ssCTX samples were combined, HTT mRNA remaining relative to vehicle was 95±3% (mean±stdev), 94±5%, and 90±5% for Group A1, Group A2 and Group A3, respectively. HTT mRNA remaining for the vehicle control Group A6 was 100±2% relative to control. Thus, HTT mRNA knockdown was about 5% for Group A1, 6% for Group A2, and 10% for Group A3. HTT mRNA levels in combined mCTX and ssCTX samples from each AAV1-VOYHT1-treated group averaged per animal after normalization to the vehicle control group are also presented in Table 29.

TABLE 29
HTT mRNA knockdown in combined mCTX and ssCTX
punches across infusion volumes averaged per animal

| | | Number | HTT mRNA |
|---|---|---|---|
| | | of | relative to vehicle |
| Group | Description | animals | (% ± stdev) |
| Group A1 | Low Vol; High Conc. | 3 | 95 ± 3 |
| Group A2 | Mid Vol; High Conc. | 3 | 94 ± 5 |
| Group A3 | High Vol; High Conc. | 3 | 90 ± 5 |
| Group A6 | Vehicle Control | 3 | 100 ± 2 |

For VG levels in combined mCTX and ssCTX punches, Group A3 showed 1.74 t 0.3 VG copies/cell (averaged per animal), a higher VG representation than observed in punches from Group A2 and Group A1, which contained 1.01±0.7 and 0.99±0.4 VG copies/cell, respectively. VG copies were below the quantification limit (BLQ) for Group A6 (vehicle control). For VG levels, the average number of VG copies detected in combined mCTX and ssCTX punches from each group averaged per animal is presented in Table 30.

TABLE 30
VG copies in combined mCTX and ssCTX punches
across infusion volumes averaged per animal

| | | | HTT mRNA |
|---|---|---|---|
| | | Number | relative to vehicle |
| Group | Description | of animals | (% ± stdev) |
| Group A1 | Low Vol; High Conc. | 3 | 0.99 ± 0.4 |
| Group A2 | Mid Vol; High Conc. | 3 | 1.0 ± 0.7 |
| Group A3 | High Vol; High Conc. | 3 | 1.74 ± 0.3 |
| Group A6 | Vehicle Control | 3 | BLQ |

Together, for mCTX and ssCTX combined samples, increasing infusion volume tracked with enhanced HTT knockdown and higher VG representation.

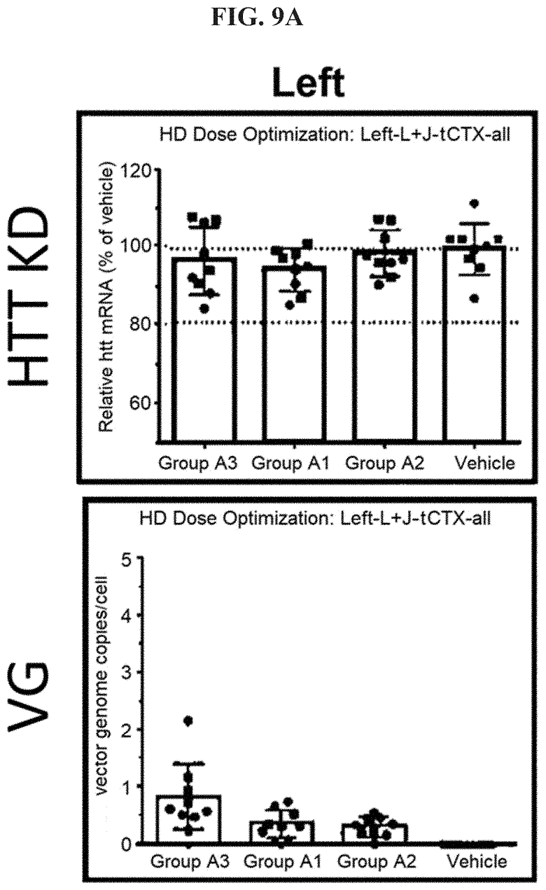

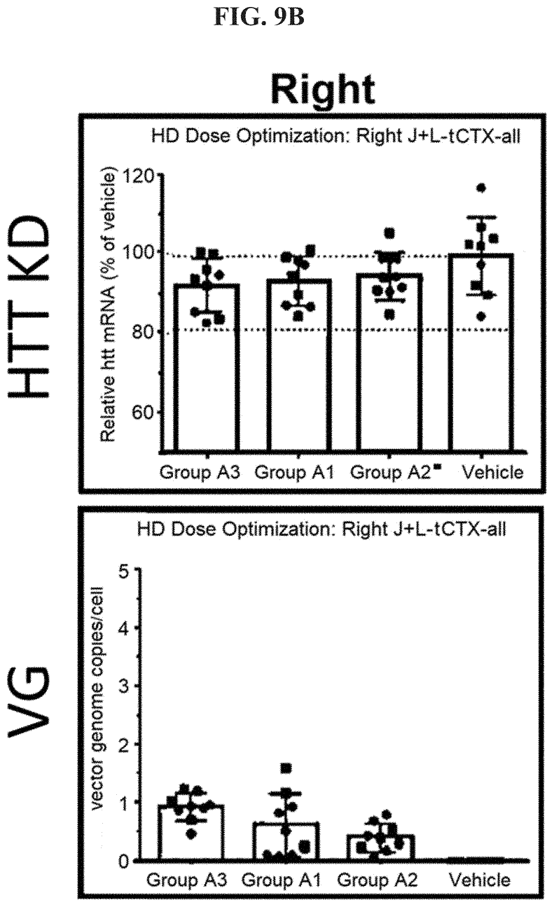

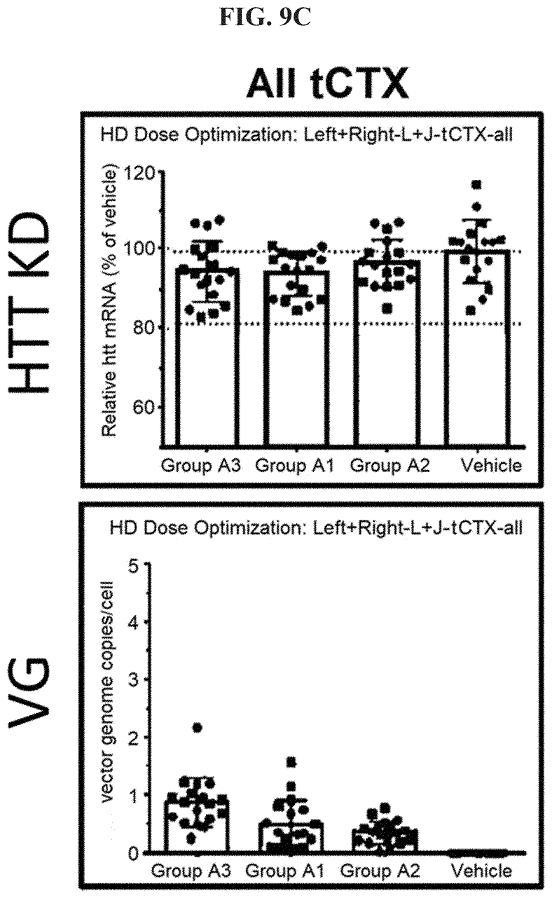

In the tCTX, no statistically significant HTT KD was seen for any of the three groups (see FIGS. 9A-9C). When VG copies were analyzed in all tCTX punches sampled from each of the three groups, lower VG representation was detected in all groups relative to other cortical areas, but Group A3 had a relatively higher VG representation than Groups A1 and A2 (see FIGS. 9A-9C).

In sum, VG was consistently detected in cortex, with the highest representation in mCTX, followed by ssCTX. Variability was observed between punches, cortical areas, and left and right hemispheres. Relatively greater HTT mRNA knockdown was observed in mCTX as compared to ssCTX and tCTX. Among the groups, Group A3 exhibited the highest VG representation and greatest HTT mRNA knockdown. A relationship between increasing VG levels and enhanced HTT mRNA knockdown was observed in cortex, as it was in the putamen and caudate.

In the thalamus, all groups demonstrated HTT mRNA knockdown with 35%, 38% and 30% HTT remaining relative to vehicle for Groups A1. A2, and A3, respectively. HTT mRNA levels in the thalamus from AAV1-VOYHT1-treated groups after normalization to the vehicle control group are presented in Table 31.

TABLE 31
HTT mRNA knockdown in thalamus punches
across infusion volumes averaged per animal

| | | | HTT mRNA |
|---|---|---|---|
| | | Number | relative to |
| | | of | vehicle |
| Group | Description | animals | (% ± sides) |
| Group A1 | Low Vol; High Conc. | 3 | 35 ± 10 |
| Grou2 A2 | Mid Vol; High Conc. | 3 | 38 ± 6 |
| Group A3 | High Vol; High Conc. | 3 | 30 ± 1 |
| Group A6 | Vehicle Control | 3 | 100 ± 2 |

For VG levels, the average number of VG copies detected in thalamus punches from each group averaged per animal is presented in Table 32. The thalamus exhibited the greatest VG representation with the largest infusion volume. Thus, Group A3 had a greater VG representation than Group A1 and A2. VG copies were below the quantification limit (BLQ) for Group A6 (vehicle control).

TABLE 32
HTT mRNA knockdown in thalamus punches
across infusion volumes averaged per animal

| | | Sample | VG copies/cell |
|---|---|---|---|
| Group | Description | size | (mean ± stdev) |
| Group A1 | Low Vol; High Conc. | 3 | 849.9 ± 112.6 |
| Group A2 | Mid Vol; High Conc. | 3 | 773.0 ± 655.3 |
| Group A3 | High Vol; High Conc. | 3 | 1136.5 ± 270.8 |
| Group A6 | Vehicle Control | 3 | BLQ |

Overall, these observations demonstrated that vector volume affects vector biodistribution in vivo. Among the tested areas, all groups displayed substantial HTT mRNA knockdown in putamen, while in caudate Group A3 led to substantial HTT knockdown. In cortex, mCTX (Groups A3 and A2) and ssCTX (Group A3) showed statistically significant HTT mRNA knockdown, which corresponded to high vector distribution. All groups demonstrated HTT mRNA knockdown in the thalamus, where VG representation was highest among all regions sampled. Lower VG representation was detected in the cortex as compared to the putamen, but relatively more VG copies were seen in mCTX than in other cortical areas. High VG levels were associated with enhanced HTT knockdown in putamen, caudate, cortex, and thalamus. Group A3 showed the highest VG distribution and demonstrated the greatest HTT mRNA knockdown of each of the four brain areas sampled. Lastly, AAV1-VOYHT1reduced HTT mRNA levels in striatum and primary motor cortex in a volume-dependent manner.

v. HIT Knockdown and Vector Genome (VG) Measurement in Punches from NHP Striatum at Mid and Low Concentrations

This analysis was designed to evaluate the impact of mid dose concentration, which can also be referred to as medium dose concentration, and low dose concentration on vector distribution and coverage. Selected brain slabs containing the motor and somatosensory cortex and anterior putamen from Groups A4 (mid concentration), A5 (low concentration), and A6 (control) were used to collect 2 mm punches. Six cortex. 8 putamen, 2 caudate, and 5 thalamus punches were collected from each side of the brain (42 total per animal), with a total number of 504 punches collected from all four groups. Samples were homogenized in QuantiGene® homogenization buffer and subjected to protease K digest. Cleared cell lysates were generated and processed for both HTT mRNA measurement using a branched DNA (bDNA) assay and vector genome (VG) measurement using digital droplet PCR (ddPCR) after an additional DNA purification step (Qiagen, catalog #69506). The bDNA assay was carried out according to the QuantiGene® Plex Assay (ThermoFisher Scientific) protocol using a probe set specifically for rhesus HTT Cell lysate was assayed in duplicate. HTT mRNA level was normalized to the geometric mean of three rhesus housekeeping genes, i.e. AARS, TBP, and XPNPEP1. Results were calibrated to the normalized mean of the vehicle group and presented as: mean of relative remaining HTT mRNA (%)±stdev. For ddPCR, whole cell DNA was prepared from same tissue homogenate used in the bDNA assay. The level of vector genome detected with probe set, CBA Promoter, was normalized to a Host probe set (RNase P). All samples were blinded during the analysis.

In the putamen, both Group A4 (mid concentration) and Group A5 (low concentration) showed HTT mRNA knockdown, with 63±9% (mean±stdev) and 73±9% HTT mRNA remaining relative to control, respectively. Thus, mRNA levels were reduced in a dose-associated manner, with an approximate 37% and 27% reduction in HTT mRNA for mid and low concentration groups, respectively. For VG levels, the average number of VG copies detected in putamen punches for Group A4 and Group A5 were 119.4±18.1 and 66.9±21.5 VG copies/cell, respectively.

HTT mRNA knockdown was also observed in the caudate, with 88±6% (mean±stdev) and 91±10% knockdown relative to control for Groups A4 and A5, respectively. Thus, mRNA levels were reduced in a dose-associated manner, with an approximate 12% and 9% reduction in HTT mRNA for mid and low concentration groups, respectively. HTT mRNA reduction was about 20% lower in the caudate versus the putamen for both Group A4 and Group A5. For VG levels, the average number of VG copies detected in caudate punches from Group A4 and Group A5 was 0.4±0.1 and 9.3±15.4 VG copies/cell, respectively. When a Grubbs' test (Q=0.1%) was applied to remove outliers, the average number of VG copies detected in caudate punches from Group A5 went from 9.3 to 0.3±0.2. Average VG for Group A4 remained unchanged after the Grubbs' test. VG copy representation was several-fold lower in the caudate than in the putamen at both medium (˜300-fold lower) and low (˜7-fold lower) dose concentrations.

Lastly, HTT mRNA knockdown was observed in the thalamus, with 59±20% (mean±stdev) and 52±13% knockdown relative to control for Groups A4 and A5, respectively. HTT mRNA levels were reduced in the thalamus by approximately 41% and 48% for mid and low concentration groups, respectively. While an emerging relationship between HTT mRNA knockdown and dose concentration was observed in the striatum, this was not the case in thalamus, where the mid dose concentration was associated with lower mRNA knockdown levels than the low dose concentration. For VG levels, the average number of VG copies detected in thalamus punches from Group A4 and A5 was 416.0±149.3 and 246.7±87 VG copies/cell, respectively. VG representation was higher in the thalamus than in the striatum at both mid and low dose concentrations.

Together, mid and low AAV1-VOYHT1 concentrations were associated with reduced HTT mRNA levels in striatum (putamen and caudate) and thalamus. HTT mRNA knockdown was higher in the thalamus compared to both the putamen and caudate. In the striatum, HTT mRNA knockdown was approximately 20% greater in the putamen versus the caudate. The medium AAV1-VOYHT1dose was associated with greater HTT knockdown than the low AAV1-VOYHT1dose in the striatum, but not in the thalamus, where knockdown was about 45% regardless of dose. Of the three brain regions assessed, the number of VG copies per cell was highest in the thalamus and lowest in the caudate.

vi. HTT Knockdown and Vector Genome (VG) Measurement in Laser Captured (LC) Neurons from NHP Cortex

Selected brain slabs from Group A3 (high vol; high conc.) and Group A6 (vehicle control) were processed to isolate primary motor cortex (mCTX) and somatosensory cortex (ssCTX) samples. Samples were cut into 14 μm sections and stained with 1% cresyl violet. Cortical pyramidal neurons were captured using laser capture microdissection (LCM). For the 1^{st} LCM analysis, one mCTX and one ssCTX sample were collected from each side of the brain (4 samples per animal), with a total of 24 samples collected. Two sets of 750 pyramidal neurons in cortex layers V and VI were laser captured (LC) and homogenized in 50 μl lysis buffer, pooled to a total of 100 μl. For the 2^{nd} LCM analysis, two mCTX and four ssCTX sample were collected from each side of the brain (12 samples per animal), with a total of 72 samples collected. Nine-hundred pyramidal neurons were laser captured from cortical layers V and VI from each sample. Each sample was initially isolated using ARCTURUS® PicoPure® RNA Isolation Kit (Thermo Fisher Scientific, Cat. No. KIT0204) and subsequently processed for both HTT mRNA level using quantitative reverse transcription-PCR (RT-qPCR) and vector genome (VG) level using digital droplet PCR (ddPCR), after an additional DNA purification step (Qiagen, catalog #56304). For RT-qPCR, all samples were analyzed with TaqMan™ PreAmp Master Mix (Thermo Fisher Scientific, Cat. No. 4391128). Calculations across the data sets were carried out according to Vandesompele J et al., Genome Biol. 2002; 3(7):RESEARCH0034. HTT mRNA level was normalized to the geometric mean of three rhesus housekeeping genes, i.e. AARS, TBP, and XPNPEP1. Results were calculated as fold HTT mRNA relative to the average of all vehicle samples in a given tissue. For ddPCR, the level of vector genome detected with probe set, CBA Promoter, was normalized to a Host probe set (RNase P). All samples were blinded during the analysis.

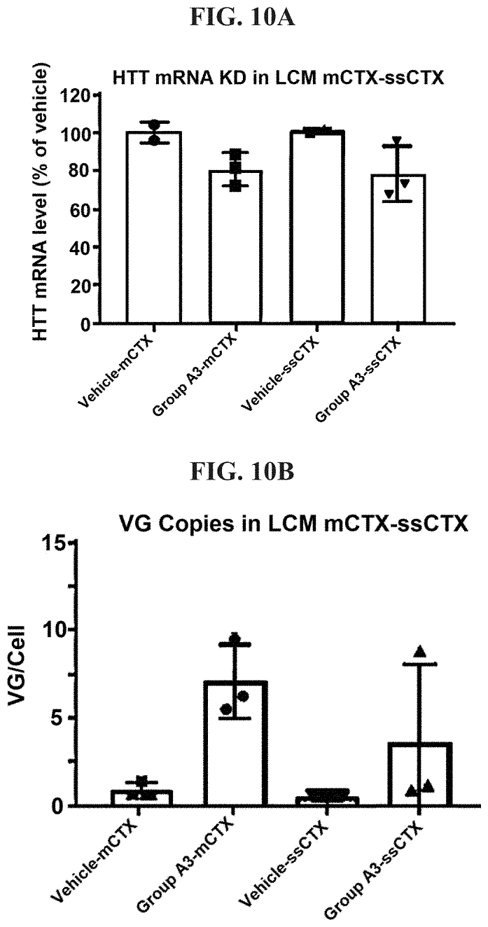

From the 1^{st} LCM analysis, modest HTT mRNA knockdown (19% in mCTX and 23% in ssCTX) was achieved in Group A3 (highest volume and concentration) (see FIG. 10A). Approximately 3-7 VG copies per cell correlated with modest HTT mRNA KD in mCTX and ssCTX pyramidal neurons (see FIG. 10B). Compared to tissue punches from mCTX and ssCTX (see above), more HTT knockdown and vector genome copies were detected in LCM samples.

From the 2^{nd} LCM analysis, HTT mRNA levels and VG levels normalized to vehicle are presented in Tables 33-36. Data shown are mean±stdev for all mCTX or ssCTX samples from a group or accounted for individual animals in a group (3 NHPs per group). Combined mCTX and ssCTX pyramidal neuron samples were also assessed for individual animals in a group, as shown in Tables 34 and 36. Modest, but significant HTT mRNA knockdown (21% in mCTX and 23% in ssCTX) was achieved in Group A3. Average 2.79 and 1.36 VG copies per cell were detected in LC pyramidal neurons from mCTX and ssCTX, respectively. Better HTT mRNA knockdown was seen in LCM samples compared to the tissue punches (14% in mCTX and 6% in ssCTX). The readouts of HTT mRNA knockdown from 2^{nd} LCM analysis was consistent with 1^{st} LCM results, while VG copy number measured in the 2^{nd} LCM was slightly lower than that in the 1^{st} LCM analysis.

TABLE 33
HTT mRNA level in all LC neurons of mCTX and ssCTX (2^{nd} LCM)

| | | Relative HTT mRNA level (% | |
|---|---|---|---|
| | | of vehicle) (mean ± stdev) | |
| Group | Description | mCTX (n = 12) | ssCTX (n = 24) |
| Group A3 | high vol; high conc. | 76 ± 11 | 77 ± 14 |
| Group A6 | vehicle control | 100 ± 37 | 100 ± 18 |

TABLE 34
HTT mRNA level in LC neurons of mCTX
and ssCTX of each animal (2^{nd} LCM)

| | | Relative HTT mRNA level (% of vehicle) | | |
|---|---|---|---|---|
| | | (mean ± stdev, N = 3) | | |
| Group | Description | mCTX | ssCTX | mCTX + ssCTX |
| Group A3 | high vol; high conc. | 79 ± 11 | 77 ± 8 | 77 ± 13 |
| Group A6 | vehicle control | 100 ± 37 | 100 ± 17 | 100 ± 25 |

TABLE 35
VG level in all LC neurons of mCTX and ssCTX (2^{nd} LCM)

| | | VG copies/cell (mean ± stdev) | |
|---|---|---|---|
| Group | Description | mCTX (n = 12) | ssCTX (n = 24) |
| Group A3 | high vol; high conc. | 2.79 ± 1.55 | 1.36 ± 0.97 |
| Group A6 | vehicle control | 0.18 ± 0.11 | 0.46 ± 0.3 |

TABLE 36
VG level in LC neurons of mCTX and
ssCTX of each animal (2^{nd} LCM)

| | | VG copies/cell (mean ± stdev, N = 3) | | |
|---|---|---|---|---|
| Group | Description | mCTX | ssCTX | mCTX + ssCTX |
| Group A3 | high vol; high conc. | 2.79 ± 0.08 | 1.36 ± 0.28 | 1.84 ± 0.17 |
| Group A6 | vehicle control | 0.17 ± 0.05 | 0.45 ± 0.7 | 0.37 ± 0.12 |

vii. In Situ Hybridization (ISH) for VG and HTT mRNA in NHP Motor and Somatosensory

Selected brain slices containing the motor and somatosensory cortex from Group A3 (high vol: high conc.) and Group A6 (vehicle control) animals were processed for in situ hybridization (ISH) using the BaseScope™ Assay to detect vector genome DNA and HTT mRNA. Five μm-thick formalin-fixed paraffin-embedded (FFPE) brain sections were incubated with BaseScope™ ISH target-specific probes for Macaca mulatta HTT mRNA (GenBank Accession Number: XM_015137840.1) and the AAV1 vector genome. Three pairs of double Z probes were used for HTT mRNA, and these probes were designed against 3 exon junctions in the HTT gene. Four pairs were used for the vector genome, and these probes were designed against multiple non-pri-miRNA regions. Positive control probes, BA-Mmu-PPIB-3zz (Peptidylprolyl Isomerase B (Cyclophilin B). Cat. No. 708161), and a negative control probe, BA-dapB-3zz (Cat. No. 701011), were also added. Signal amplification and tissue staining were carried out using BaseScope™ Red Reagent Kit (Cat. No. 322910). Images were detected and analyzed under a microscope for vector genome and HTT mRNA levels.

Quantification of BaseScope™ ISH results was performed with ImageJ imaging analysis software. The scoring criteria used for evaluation of BaseScope™ staining is listed in Table 37. Scoring was performed at 40× magnification. Scoring was performed based on the number of dots per cell rather than the signal intensity, since dots correlate to the number of individual target molecules, whereas dot intensity reflects the number probe pairs bound to each molecule. AAV vector biodistribution was calculated as the percentage of cells with dots relative to the total number of cells in a specific cortex region. For vector genome readouts, only nuclear signals were counted.

TABLE 37
Scoring criteria for ISH staining

| Score | Criteria |
|---|---|
| 0 | No staining or < 1 dot/10 cells |
| 1 | 1-3 dots/cell |
| 2 | 4-9 dots/cell, no or very few dot clusters |
| 3 | 10-15 dots/cell and < 10% dots are in clusters |
| 4 | >15 dots/cell and > 10% dots are in clusters |

For vector biodistribution, extensive vector genomes were detected at the injection sites (thalamus and putamen) in Group A3. In the cortex, an average of 18% mCTX and 9% ssCTX cells with detectable AAV vector in the nucleus was achieved in Group A3. More cells with detectable vector genome were observed in the mCTX than in the ssCTX, with a combined average of 12.48% vg+cells in both the mCTX and ssCTX in NHPs. The results of vector biodistribution in NHP cortex based on vector genome ISH are presented in Tables 38 and 39.

TABLE 38
VG distribution per cortical region in mCTX and ssCTX

| | | % of cells with VG | |
|---|---|---|---|
| | | (mean ± stdev) | |
| Group | Description | mCTX (n = 18) | ssCTX (n = 30) |
| Group A3 | high vol; high conc. | 17.87 ± 7.04 | 9.35 ± 2.80 |
| Group A6 | vehicle control | 0.75 ± 1.51 | 0.72 ± 0.92 |

TABLE 39
VG distribution per animal in mCTX and ssCTX

| | | % of cells with VG | |
|---|---|---|---|
| | | (mean ± stdev, N = 3) | |
| Group | Desetiption | mCTX | ssCTX |
| Group A3 | high vol; high conc. | 17.87 ± 2.94 | 9.36 ± 0.29 |
| Group A6 | vehicle control | 0.75 ± 1.12 | 0.72 ± 0.53 |

For VG levels, average vector genome scorings were ˜1 for cells in both mCTX and ssCTX in NHPs of Group A3 (high volume, high concentration) dosed with AAV1-VOYHT1 by bilateral thalamus and putamen infusion. The results of VG scoring in NHP cortex using the scoring criteria set forth above are presented in Tables 40 and 41.

TABLE 40
VG score per cortical region in mCTX and ssCTX

| | | VG score (mean ± stdev) | |
|---|---|---|---|
| | | mCTX | ssCTX |
| Group | Description | (n = 18) | (n = 31) |
| Group A3 | high vol; high conc. | 1.01 ± 0.02 | 1.08 ± 0.13 |
| Group A6 | vehicle control | 0 | 0 |

TABLE 41
VG score per animal in mCTX and ssCTX

| | | VG score | |
|---|---|---|---|
| | | (mean ± stdev, N = 3) | |
| Group | Description | mCTX | ssCTX |
| Group A3 | high vol; high conc. | 1.01 ± 0.01 | 1.08 ± 0.07 |
| Group A6 | vehicle control | 0 | 0 |

For HTT mRNA levels, HTT mRNA scores in both mCTX and ssCTX of the AAV1-VOYHT1-treated group showed significantly lower scores than that in the vehicle group, indicating a significant reduction of HTT mRNA level caused by AAV1-VOYHT1 treatment. The results of HTT mRNA scoring in NHP cortex using the scoring criteria set forth above are presented in Tables 42 and 43.

TABLE 42
HTT mRNA scores per cortical region in mCTX and ssCTX

| | | HTT mRNA scores | |
|---|---|---|---|
| | | (mean ± stdev) | |
| | | mCTX | ssCTX |
| Group | Description | (n = 18) | (n = 30) |
| Group A3 | high vol; high conc. | 1.51 ± 0.22 | 1.91 ± 0.28 |
| Group A6 | vehicle control | 2.24 ± 0.38 | 2.26 ± 0.28 |

TABLE 43
HTT mRNA scores per animal in mCTX and ssCTX

| | | HTT mRNA score | |
|---|---|---|---|
| | | (mean ± stdev, N = 3) | |
| Group | Description | mCTX | ssCTX |
| Group A3 | high vol; high conc. | 1.51 ± 0.15 | 1.91 ± 0.25 |
| Group A6 | vehicle control | 2.24 ± 0.28 | 2.26 ± 0.2 |

viii. Clinical Signs and Histopathology

Minimal to mild clinical signs were observed in 7 out of 18 test animals, including mild incoordination, inappetence, decreased feed, and overall weakness. Histopathology analysis overall showed safety at the tested doses. Low levels of mononuclear cell infiltrates were detected in the putamen and thalamus. The degree of infiltration of mononuclear cells corresponded proportionately to the infusion volume. Necrosis was most pronounced in the vehicle group. Minimal damage was observed in parietal cortex and occipital cortex.

ix. Summary

These data suggested that attaining the targeted levels of HTT knockdown in cortex via intrathalamic and/or intraputamenal infusion is achievable upon using optimal dosing paradigm. AAV1-VOYHT1 was well-tolerated based on clinical signs and histological assessment of the brain.

### Example 19. Dose Optimization Study II

This study was carried out to further evaluate delivery parameters to optimize coverage of AAV1-VOYHT1 in NHP brain, and to extrapolate parameters to clinical dosing paradigm. This study utilized a total of 10 animals, which were assigned to 4 treatment groups as summarized in Table 44. Animals received bilateral parenchymal infusion (4 infusions) into the putamen and thalamus of AAV1-VOYHT1 with increased dosing compared to Example 18. Experimental procedures were similar to that described in Example 18. Animals were euthanized 5 weeks after dosing, and tissues were collected for post-mortem analysis.

TABLE 44
Study design

| | | Number of | Volume (μL/side) | | Dosing Titer | Total |
|---|---|---|---|---|---|---|
| Group | Description | animals | Putamen | Thalamus | (vg/ml) | dose (vg) |
| Group B1 | High vol. vehicle | 2 | 350 | 500 | 0 | 0 |
| Group B2 | Mid vol. Mid Conc. | 3 | 250 | 325 | 4e12 | 4.6e12 |
| Group B3 | Mid vol. High Conc. | 3 | 250 | 325 | 7.9e12 | 9.1e12 |
| Group B4 | Mid vol. Vehicle | 2 | 250 | 325 | 0 | 0 |

The calculated human equivalent dose corresponding to each dosing group from Table 44 is presented in Table 45.

TABLE 45
Human equivalent dose

| | | Human Equivalent Dose | | |
|---|---|---|---|---|
| | | Putamen | Thalamus | dose |
| Group | Description | (μL/side) | (μL/side) | (vg) |
| Group B1 | High vol. vehicle | 1225 | 3000 | 0 |
| Group B2 | Mid vol. Mid Conc. | 875 | 1950 | 2.3e13 |
| Group B3 | Mid vol. High Conc. | 875 | 1950 | 4.5e13 |
| Group B4 | Mid vol. Vehicle | 875 | 1950 | 0 |

Side effects were observed post-dosing, which were likely due to intolerance to large infusion volumes. Disuse of one or both hindlimbs was observed in the animals dosed with the high-volume vehicle control (Group B1). In two animals that received medium volume AAV1-VOYHT1 treatment (Groups B2, B3), clinical signs such as paresis in both legs, prone/ambulating slowing, head tilt were observed. MRI observations showed some reflux along both cannula tracts in three animals.

Histopathology analysis showed slight gliosis and necrosis in the putamen (unavoidable due to placement of catheter) in the vehicle group. In Group B2 animals, a notable increase was seen in mononuclear cell infiltrates at both putamen and thalamic infusion sites, but these were not expected to result in clinical signs. Slight increases in gliosis and necrosis in both structures were observed but neither finding was expected to result in any clinical signs. Edema was also observed. In Group B3 animals, slight increases in gliosis and necrosis were seen in both structures relative to control but were considered of no biologic relevance. Mononuclear infiltrates were increased compared to the vehicle group. An increase in edema was also observed, but this was not expected to cause any clinical signs.

### Example 20. Dose Optimization Study III

i. Study Design

The primary goals of this study were to demonstrate HTT mRNA knockdown in NHP cortex with AAV1-VOYHT1 and to demonstrate safety for thalamic-only and combined thalamic and putaminal infusion paradigms. The secondary goals were to show a correlation between VG and HTT mRNA levels in laser captured (LC) pyramidal neurons from primary motor and somatosensory cortex; demonstrate a correlation between HTT mRNA and VG levels in tissue punches from the putamen, thalamus, and caudate; demonstrate a correlation between HTT protein and HTT mRNA levels in putamen; measure AAV1-VOYHT1 specific miRNA expression levels in tissue punches from putamen and caudate: demonstrate a correlation between vector genome (VG) and AAV1-VOYHT1 specific miRNA expression levels in tissue punches from putamen and caudate; and demonstrate a correlation between HTT mRNA and AAV1-VOYHT1 specific miRNA expression levels in tissue punches from putamen and caudate.

This study was implemented in two phases. A total of 15 male and female rhesus macaques were assigned to 5 groups with 3 animals per group (see Table 34). In Phase I, the first group of animals (Group C1a) were dosed with a vehicle control by intraparenchymal injection bilaterally into both the thalamus and putamen using MRI-guided convection enhanced delivery (CED) to establish infusion parameters (e.g., rate, volume and duration) before proceeding to Phase II of the study. A second group (Group C1b) was dosed with refined surgical procedures and served as the control group for the treatment groups. After the infusion parameters were established in Phase I, they were used for dosing the test article containing AAV1-VOYHT1 in the three treatment groups. The first treatment group (Group C2) received bilateral infusion of the test article into the thalamus only using MRI-guided CED. This group was dosed to demonstrate the safety and Huntingtin (HTT) mRNA knockdown (KD) in cortical pyramidal neurons in the primary motor and somatosensory cortex by laser capture microdissection (LCM) after thalamus infusion only. Next, in the other two treatment groups (Group C3 and Group C4), the test article was infused bilaterally into both the thalamus and putamen at 2 different dose levels for dose optimization.

The study schedule was as follows. In Phase I, the first vehicle group (Group C1a) was dosed using pre-selected infusion parameters. A Functional Observation Battery (FOB) evaluation focusing on neurological status was carried out 5±2 days post-infusion and 3 t days prior to termination. An additional 3 animals (Group C1b) were dosed and then evaluated with FOB at 5±2 days after dosing and 3±days prior to termination, as per Group C1a. In Phase II, all animals (N=9) were dosed with the test article containing AAV1-VOYHT1 in accordance with the infusion parameters established in Phase I. Group C2 (thalamus only) was dosed first, followed by Group C3 at a medium dose and then Group C4 at a high dose. Except for Group C2 in which the animals received bilateral thalamic dosing only, each animal received bilateral intracranial infusion of vehicle or test article into the putamen and thalamus. An intraparenchymal dosing paradigm was employed in which 2-4 infusions (1 infusion per structure) were given at a speed of up to 5 μL/min. A baseline neurological FOB evaluation was performed on each animal prior to dosing followed by a second FOB evaluation of each animal at 5±2 days after dosing. When the second FOB was satisfactory, the animal was euthanized at Day 36±3 (˜5 weeks in-life duration) and a third FOB evaluation was performed 3±2 days prior to necropsy. Tissues were collected for post-mortem analysis.

A summary of the study design is shown in Table 46. For the high dose group (Group C4), the total dose of 1.8e13 vg was calculated based on the maximal titer (2.2e13 vg/ml) achieved.

TABLE 46
Study design

| | | Number | | | | | |
|---|---|---|---|---|---|---|---|
| | | of | | Volume (μL/side) | | Dosing Titer | Total |
| Phase | Group | animals | Description | Putamen | Thalamus | (vg/ml) | dose (vg) |
| I | Group C1 (a/b) | 3 + 3 | Vehicle | 150 | 250 | 0.0 | 0.0 |
| II | Group C2 | 3 | Thalamus only | — | 250 | 2.2e13 | 1.1e13 |
| | Group C3 | 3 | Medium dose | 150 | 250 | 1.0e13 | 8.0e12 |
| | Group C4 | 3 | High dose | 150 | 250 | 2.2e13 | 1.8e13 |

ii. Animal Care and Sample Collection

Eighteen (N=18) adult male or female rhesus macaques (4-11 years old) were selected based on anti-AAV1 neutralizing antibody (nAb) serum titers 15 days prior to the start of ambulation training. Selected candidates for Groups C2. C3 and C4 exhibited low AAV1 nAb in general. Animals weighed 5-14 kg. Ambulation training was carried out daily for up to 4 consecutive weeks before animal enrollment. Animals were weighed and assigned by nAb status to the study groups as summarized in Table 34. The 3 animals selected as backups were kept as spares until the completion of dosing. Animal husbandry conditions were similar as described in Example 18.

Blood samples were collected for clinical pathology evaluation and neutralizing antibody (nAb) analysis at Day 1 (Pre-dose), Day 15±2, and immediately prior to necropsy on Day 36±3. The clinical pathology evaluation included hematology (CBC), serum clinical chemistry (Chem), and coagulation (Coag) analysis. Cerebrospinal fluid (CSF) samples were collected for nAb analysis from the cervical region at Day 1 (Pre-dose), Day 15±2, and immediately prior to necropsy on Day 36±3. Following necropsy, the brain and selected peripheral organs were collected and then fresh frozen or 4% paraformaldehyde (PFA) post-fixed by immersion.

iii. Test Article Preparation and Dosing Procedures

The test article used in the study contained AAV1-VOYHT1 gene transfer vector formulated in Phosphate Buffered Saline with 5% sucrose and 0.001% Poloxamer 188 (Pluronic® F-68). The vehicle control contained the formulation buffer only. The samples were stored at −60° C. or below and were thawed to and maintained at 2-8° C. on day of dosing. ProHance® (Bracco Diagnostics, Inc). i.e. gadoteridol, was added at a 1:250 ratio (1 μL of ProHance per 250 μL of test article or control) and carefully mixed by inverting tubes prior to loading into the infusion system. The dosing solution contained the test article or control and a 2 mM concentration of gadoteridol.

Immediately prior to surgery, each animal was anesthetized with intramuscular (IM) ketamine (10 mg/kg) and IM dexmedetomidine (15 μg/kg), weighed, hair of head and neck shaved, intubated, and maintained on 1-5% isoflurane. The animal's head was secured onto a stereotaxic frame containing one MRI surface coil on each side of the ear bars and then transferred to the MRI to acquire a baseline scan. A T1- and T2-weighted MRI sequences were acquired and used to determine coordinates of the central sulcus. Next the animal was transferred back to the surgery suite and the head prepared for the neurosurgical implantation procedure. Using aseptic techniques, the wound site was opened in anatomical layers to expose the skull. Depending on which dose group, craniotomies were performed at entry sites located above the parietal and/or occipital lobe on each side. A skull mounted cannula guide ball array was temporarily secured to the skull over each burr hole using titanium screws. Immediately after implantation of ball arrays, the animal was transferred to the MRI suite. MR imaging was used to align cannula guides with putamen and/or thalamus targets ipsilateral to each cannula guide. Test article or control was administered with repeated MR imaging to visually monitor infusions within the brain as specified in Table 34 (above). Each animal received infusions (sites) of the test article or control using convection enhanced delivery (CED) in each putamen (except Group C2) and thalamus. A 16G cannula (MRI Interventions Inc.) was primed with dosing solution and guided into each target site through the skull mounted ball arrays. Each cannula was connected via microbore extension lines (Smiths Medical) to a 3-6 cc syringe mounted on a syringe pump (Harvard Apparatus). Dose rates, durations and volumes administered into each putamen and thalamus using ascending infusion rates in 3 different stages of intraparenchymal infusion are listed in Table 47. “N/A” indicates data not applicable.

TABLE 47
Infusion parameters

| | Putamen | | | Thalamus | | |
|---|---|---|---|---|---|---|
| Stage of | Rate | Duration | Volume | Rate | Duration | Volume |
| infusion | (μl/min) | (min) | (μl) | μl/min) | (mm) | (μl) |
| 1 | 1 | 16 | 16 | 1 | 25 | 25 |
| 7 | 3 | 28 | 84 | 3 | 50 | 150 |
| 3 | 5 | 20 | 50 | 5 | 15 | 75 |
| Total | N/A | 64 | 150 | N/A | 90 | 250 |

Serial MRI scans were acquired to monitor infusate distribution within each target site and provide real-time monitoring of the dosing. In some cases, cannula was advanced deeper into the putamen or thalamus during the infusion to maximize infusate distribution within the putamen or thalamus. Distribution of infusate into CNS structures adjacent to the putamen and thalamus was anticipated and intended to occur due to the total volume to be delivered per site. Immediately after the MRI CED dosing procedure, the animal was transferred back to the surgery suite, the ball array system was explanted and the wound site closed in anatomical layers with absorbable vicryl suture using a simple interrupted suturing pattern. Pre- and post-operative medications included atipamezole (0.03 mL/kg, IM), buprenorphine (0.03 mg/kg, IM, b.i.d.), carprofen (2.2 mg/kg SQ, b.i.d.), ketoprofen (2 mg/kg, IM, s.i.d.), and cefazolin (100 mg IV, pre- and post-surgery, followed by 25 mg/kg, IM, b.i.d.) or ceftriaxone (50 mg/kg, IM, s.i.d.). Animals were monitored for full recovery from anesthesia and returned to their home cages.

iv. HTT Knockdown and VG Measurement in LC Neurons from Combined NHP mCTX and ssCTX

Selected brain slabs from three groups (Group C1, Group C3 and Group C4) were processed to isolate primary motor cortex (mCTX) and somatosensory cortex (ssCTX) samples by laser capture microdissection (LCM). A total of 54 mCTX samples and 90 ssCTX LCM samples were collected. Each LCM sample contained 900 pyramidal neurons laser captured (LC) from cortical layers V and VI, with a total of 129,600 neurons captured. Samples were processed for both HTT mRNA level using RT-qPCR and vector genome (VG) level using ddPCR, as described in Example 18. All samples were blinded during the analysis.

For HTT mRNA knockdown, the relative HTT mRNA levels in LC neurons from combined mCTX and ssCTX of AAV1-VOYHT1-treated groups after normalization to the vehicle control group are presented in Table 46. The greatest HTT knockdown in combined samples of LC pyramidal neurons from mCTX and ssCTX (32%) was observed in Group C4 (high dose bilateral putamen+thalamus group), with less HTT knockdown (13%) in Group C3 (medium dose bilateral putamen+thalamus group). An average of 30% HTT mRNA knockdown was observed in mCTX and 33% HTT mRNA knockdown in ssCTX in Group C4. HTT knockdown was approximately dose-proportional (2.25× greater dose resulted in 2.9× greater knockdown). The percentage of samples of LC cortical neurons that exhibited over 30% HTT knockdown is also shown in Table 48. In LC motor and somatosensory cortical neurons, 58% of samples showed ≥30% HTT knockdown and 27% of samples showed ≥40% HTT knockdown in Group C4 (high dose putamen+thalamus group), whereas 36% of samples showed ≥30% HTT knockdown and 7% of samples showed ≥40% HTT knockdown in Group C3 (medium dose putamen+thalamus group). Thus, HTT mRNA knockdown in motor and somatosensory cortical neurons is dependent on the concentration of AAV1-VOYHT1 infused into thalamus and putamen. In addition, over 40% of LCM mCTX samples showed ≥30% HIT mRNA knockdown in both medium and high dose groups, while 60% of LCM ssCTX samples in the high dose group showed ≥30% HTT mRNA knockdown.

TABLE 48
HTT knockdown in LC neurons from combined mCTX and ssCTX

| | | HTT mRNA | | |
|---|---|---|---|---|
| | | relative to vehicle | % LCMs | % LCMs |
| Group | Description | (% + stdev, N = 3) | ≥30% KD | ≥40% KD |
| Group C1 | Vehicle | 100 ± 10 | 5 | 0 |
| Group C3 | Medium dose | 87 ± 26 | 36 | 7 |
| Group C4 | High dose | 68 ± 3 | 58 | 27 |

For VG levels, LC neuron samples from combined mCTX and ssCTX showed a dose-dependent increase in VG copies per cell, as shown in Table 49. The number of VG copies per cell was approximately 30 copies/cell in the high dose group. VG copies tracked with HTT mRNA knockdown such that a higher number of VG copies corresponded to greater HTT mRNA knockdown.

TABLE 49
VG levels in LC neurons from combined mCTX and ssCTX

| | | VG copies/cell |
|---|---|---|
| Group | Description | (mean ± stdev, N = 3) |
| Group C1 | Vehicle | 0.54 ± 0.55 |
| Group C3 | Medium dose | 8.00 ± 0.71 |
| Group C4 | High dose | 28.55 ± 23.16 |

v. HTT Knockdown and VG Measurement in LC Neurons from NHP mCTX

Selected brain slabs from three groups (Group C1, Group C3 and Group C4) were processed to isolate primary motor cortex (mCTX) samples by laser capture microdissection (LCM). A total of 54 mCTX samples were collected. Each LCM sample contained 900 pyramidal neurons laser captured (LC) from cortical layers V and VI. Samples were processed for HTT mRNA level using RT-qPCR and vector genome (VG) level using ddPCR, as described in Example 18. All samples were blinded during the analysis.

For HTT mRNA knockdown, the relative HTT mRNA levels in LC neurons from mCTX of AAV1-VOYHT1-treated groups after normalization to vehicle control group are presented in Table 50. The greatest HTT knockdown in LC pyramidal neurons from mCTX (30%) was observed in Group C4 (high dose bilateral putamen+thalamus group), with less HTT knockdown (13%) in Group C3 (medium dose bilateral putamen+thalamus group).

TABLE 50
HTT knockdown in LC neurons from mCTX

| | | HTT mRNA |
|---|---|---|
| | | relative to vehicle |
| Group | Description | (mean ± stdev, N = 3) |
| Group C1 | Vehicle | 100 ± 11 |
| Group C3 | Medium dose | 87 ± 27 |
| Group C4 | High dose | 70 ± 7 |

For VG levels, LC neurons from mCTX showed a dose-associated increase in VG copies per cell, as shown in Table 51. The number of VG copies per cell reached approximately 20 copies/cell in Group C4 (high dose bilateral putamen+thalamus group), and 10 copies/cell in Group C3 (medium dose bilateral putamen+thalamus group). Thus, VG copies tracked with HTT mRNA knockdown such that a higher number of VG copies corresponded to greater HTT mRNA knockdown

TABLE 51
VG levels in LC neurons from mCTX

| | | VG copies/cell |
|---|---|---|
| Group | Description | (mean ± stdev, N = 3) |
| Group C1 | Vehicle | 0.15 ± 0.03 |
| Group C3 | Medium dose | 9.61 ± 1.59 |
| Group C4 | High dose | 21.6 ± 0.74 |

vi. HTT Knockdown and VG Measurement in LC Neurons from NHP ssCTX

Selected brain slabs from three groups (Group C1, Group C3 and Group C4) were processed to isolate somatosensory cortex (ssCTX) samples by laser capture microdissection (LCM). A total of 90 ssCTX LCM samples were collected. Each LCM sample contained 900 pyramidal neurons laser captured (LC) from cortical layers V and VI. Samples were processed for HTT mRNA level using RT-qPCR and vector genome (VG) level using ddPCR, as described in Example 18. All samples were blinded during the analysis.

For HTT mRNA knockdown, the relative HTT mRNA levels in LC neurons from ssCTX of AAV1-VOYHT1-treated groups after normalization to vehicle control group are presented in Table 52. The greatest HTT knockdown in LC pyramidal neurons from ssCTX (33%) was observed in Group C4 (high dose bilateral putamen+thalamus group), with less HTT knockdown (13%) in Group C3 (medium dose bilateral putamen+thalamus group).

TABLE 52
HTT knockdown in LC neurons from ssCTX

| | | HTT mRNA |
|---|---|---|
| | | relative to vehicle |
| Group | Description | (mean ± stdev, N = 3) |
| Group C1 | Vehicle | 100 ± 8 |
| Group C3 | Medium dose | 86 ± 24 |
| Group C4 | High dose | 67 ± 11 |

For VG levels, LC neurons from sCTX showed a dose-associated increase in VG copies per cell, as shown in Table 53. The number of VG copies per cell reached approximately 33 copies/cell in the Group C4 (high dose bilateral putamen+thalamus group), and 7 copies/cell in Group C3 (medium dose bilateral putamen+thalamus group). Thus, VG copies tracked with HTT mRNA knockdown such that a higher number of VG copies corresponded to greater HTT mRNA knockdown.

TABLE 53
VG levels in LC neurons from ssCTX

| | | VG copies/cell |
|---|---|---|
| Group | Description | (mean ± stdev, N = 3) |
| Group C1 | Vehicle | 0.77 ± 0.89 |
| Group C3 | Medium dose | 7.01 ± 2.98 |
| Group C4 | High dose | 32.72 ± 37.37 |

The LCM results demonstrated that combined bilateral putaminal and thalamic infusion of AAV1-VOYHT1 resulted in VG delivery and HTT mRNA knockdown in motor and somatosensory cortical pyramidal neurons in medium and high dose groups, with greater vector genome delivery and greater HTT mRNA knockdown in the high dose group.

vii. HTT Knockdown and VG Measurement in Punches from Combined NHP mCTX and ssCTX

Two selected brain slabs containing the motor and somatosensory cortex from all four groups were used to collect 2 mm primary motor cortex (mCTX) and somatosensory cortex (ssCTX) punches. Six mCTX and 6 ssCTX punches were collected per animal, with a total number of 144 punches collected. Equal numbers of punches were collected from each side of the cortex. Samples were processed and analyzed for HTT mRNA and vector genome (VG) using bDNA and ddPCR, respectively. bDNA and ddPCR were carried out as described in Example 18. All samples were blinded during the analysis.

For HTT mRNA knockdown, the relative HTT mRNA levels in LC neurons from combined mCTX and ssCTS in the AAV1-VOYHT1-treated groups after normalization to the vehicle control group are presented in Table 54. An average of 16% HTT knockdown was observed in cortical punches from Group C4 (high dose bilateral putamen+thalamus group). HTT knockdown was dose-proportional (2.25× greater dose resulted in 2.28× greater knockdown) based on Groups C3 and C4. The percentage of punches that exhibited over 20% HTT knockdown in each group is also shown in Table 54. 39% of combined mCTX and ssCTX punches showed ≥20% HTT knockdown, but 72% of mCTX punches showed ≥20% HTT knockdown. Greater HTT knockdown was observed in the high dose putamen+thalamus Group C4 than in the thalamus only Group C2, suggesting that putamen infusion of AAV1-VOYHT1 contributes to HTT knockdown in motor and somatosensory cortex. With thalamus only infusion of AAV1-VOYHT1 (Group C2), there was no significant HTT knockdown in motor and somatosensory cortex.

TABLE 54
HTT knockdown in combined mCTX and ssCTX punches

| | | HTT mRNA | |
|---|---|---|---|
| | | relative to vehicle | % Punches |
| Group | Description | (% ± stdev, N = 3 | ≥20% KD |
| Group C1 | Vehicle | 100 ± 8 | 0 |
| Group C2 | Thalamus Only | 93 ± 3 | 14 |
| Group C3 | Medium dose | 93 ± 13 | 11 |
| Group C4 | High dose | 84 ± 5 | 39 |

For VG levels, results are summarized in Table 55. VG levels were dose-dependent and dose-proportional (2.25× greater dose resulted in 3-fold higher vector genome level) for putamen+thalamus groups C3 and C4. Higher VG copies were detected in mCTX than in ssCTX in each group. Higher VG copies were detected in the Group C4 group (high dose putamen+thalamus) than in the Group C2 group (thalamus only), suggesting that putamen infusion of AAV1-VOYHT1 contributes to VG copies in motor and somatosensory cortex. VG copies correlated with HTT mRNA knockdown in the punch analysis.

TABLE 55
VG levels in combined mCTX and ssCTX punches

| | | VG copies/cell |
|---|---|---|
| Group | Description | (mean ± stdev, N = 3) |
| Group C1 | Vehicle | 0.02 ± 0.04 |
| Group C2 | Thalamus only | 10.15 ± 4.14 |
| Group C3 | Medium dose | 10.98 ± 6.69 |
| Group C4 | High dose | 22.67 ± 10.69 |

viii. HTT Knockdown and VG Measurement in Punches from NHP mCTX

Two selected brain slabs containing the motor cortex from all four groups were used to collect 2 mm primary motor cortex (mCTX) punches. Six mCTX punches were collected per animal, with a total number of 72 punches collected. Equal numbers of punches were collected from each side of the cortex. Samples were processed and analyzed for HTT mRNA and vector genome (VG) using bDNA and ddPCR, respectively. bDNA and ddPCR were carried out as described in Example 18. All samples were blinded during the analysis.

For HTT mRNA knockdown, the relative HTT mRNA levels in mCTX punches of AAV1-VOYHT1-treated groups after normalization to vehicle control group are presented in Table 56. The greatest HTT knockdown (28%) was observed in Group C4 (high dose bilateral putamen+thalamus group), with less HTT knockdown (9%) in Group C3 (medium dose bilateral putamen+thalamus group) and Group C2 (10%; thalamus only). While an approximate one-third reduction in HTT mRNA was observed with high dose infusion into bilateral putamen and thalamus, only about a 10% reduction was seen with medium dose infusion into bilateral putamen, as well as with thalamus only infusion.

TABLE 56
HTT knockdown in mCTX punches

| | | HTT mRNA |
|---|---|---|
| | | relative to vehicle |
| Group | Description | (mean ± stdev, N = 3) |
| Group C1 | Vehicle | 100 ± 11 |
| Group C2 | Thalamus only | 90 ± 2 |
| Group C3 | Medium dose | 91 ± 11 |
| Group C4 | High dose | 78 ± 7 |

For VG levels, mCTX punches showed a dose-associated increase in VG copies/cell, as shown in Table 57. The number of VG copies per cell was approximately 32 copies/cell in Group C4 (high dose bilateral putamen+thalamus group), and 14 copies/cell in Group C3 (medium dose bilateral putamen+thalamus group). Similar to Group C3, approximately 13 vg copies/cell were seen in Group C2 (thalamus only). In general, VG levels tracked with HTT mRNA knockdown in mCTX punches such that a higher number of VG copies corresponded to greater HTT mRNA knockdown.

TABLE 57
VG levels in mCTX punches

| | | VG copies/cell |
|---|---|---|
| Group | Description | (mean ± stdev, N = 3) |
| Group C1 | Vehicle | 0.0 ± 0.00 |
| Group C2 | Thalamus only | 12.5 ± 4.8 |
| Group C3 | Medium dose | 13.8 ± 7.1 |
| Group C4 | High dose | 31.8 ± 5.7 |

ix. HTT Knockdown and VG Measurement in Punches from NHP ssCTX

Two selected brain slabs containing the somatosensory cortex from all four groups were used to collect 2 mm somatosensory cortex (ssCTX) punches. Six ssCTX punches were collected per animal, with a total number of 72 punches collected. Equal numbers of punches were collected from each side of the cortex. Samples were processed and analyzed for both HTT mRNA and vector genome (VG) using bDNA and ddPCR, respectively. bDNA and ddPCR were carried out as described in Example 18. All samples were blinded during the analysis.

For HTT mRNA knockdown, the relative HTT mRNA levels in ssCTX punches of AAV1-VOYHT1-treated groups after normalization to vehicle control group are presented in Table 58. The greatest HTT knockdown (9%) was observed in Group C4 (high dose bilateral putamen+thalamus group), with less HTT knockdown (5%) in Group C3 (medium dose bilateral putamen+thalamus group) and Group C2 (4%; thalamus only). HTT mRNA knockdown in Group C4 was approximately double that observed in Groups C3 and C2.

TABLE 58
HTT knockdown in ssCTX punches

| | | HTT mRNA |
|---|---|---|
| | | relative to vehicle |
| Group | Description | (mean ± stdev, N = 3) |
| Group C1 | Vehicle | 100 ± 7 |
| Group C2 | Thalamus only | 96 ± 6 |
| Group C3 | Medium dose | 95 ± 16 |
| Group C4 | High dose | 91 ± 6 |

For VG levels, mCTX punches showed a dose-associated increase in VG copies per cell, as shown in Table 59. The number of VG copies/cell was approximately 14 copies/cell in Group C4 (high dose bilateral putamen+thalamus group), and 8 copies/cell in Group C3 (medium dose bilateral putamen+thalamus group). Like Group C3, approximately 8 VG copies/cell were seen in Group C2 (thalamus only).VG levels generally tracked with HTT mRNA knockdown in mCTX punches such that a higher number of VG copies corresponded to greater HTT mRNA knockdown.

TABLE 59
VG levels in ssCTX punches

| | | VG copies/cell |
|---|---|---|
| Group | Description | (mean ± stdev, N = 3) |
| Group C1 | Vehicle | 0.0 ± 0.15 |
| Group C2 | Thalamus only | 7.8 ± 1.8 |
| Group C3 | Medium dose | 8.1 ± 6.1 |
| Group C4 | High dose | 13.5 ± 1.5 |

x. In Situ Hybridization (ISH) for VG and HTT mRNA in NHP Motor and Somatosensory Cortex

Selected brain slabs containing the motor and somatosensory cortex from Group C1 (vehicle group) and Group C4 (high dose−putamen+thalamus group) animals were processed for in situ hybridization (ISH) using the BaseScope™ Assay to detect vector genome DNA and HTT mRNA as described in Example 18. Images were detected and analyzed under a microscope for vector genome and HTT mRNA levels.

Extensive VGs were detected in the nucleus of cells at infusion sites (thalamus). VGs were also detected in multiple different layers of motor and somatosensory cortex (mostly pyramidal neurons). Substantial VG signal was detected in the nucleus of motor and sensory cortical neurons layers I-VI after AAV1-VOYHT1 treatment

Substantial HTT mRNA reduction was observed in cells at the infusion site (thalamus). Putamen was not significantly contained within the brain slices analyzed for ISH. ISH results demonstrated broad AAV1-VOYHT1 distribution in all NHP cortex layers and infusion sites, and confirmed HTT mRNA reduction in these regions. ISH results support HTT lowering in motor and somatosensory cortex and transduction of neurons in multiple layers of these regions.

xi. HTT Knockdown. VG Measurement and AAV1-VOYHT1 Specific miRNA Expression in Punches from NHP Putamen

Two selected brain slabs containing the putamen from all four groups were used to collect 2 mm putamen punches. Five punches were collected from each side of one slab and 3 punches were collected from each side of the other slab, with a total of 16 punches collected from each animal. A total of 192 putamen punches were collected from all 12 animals. Samples were processed and analyzed for HTT mRNA levels and VG levels using bDNA and ddPCR, respectively. bDNA and ddPCR were carried out as described in Example 18. Samples were processed and analyzed for AAV1-VOYHT1 specific miRNA levels using deep sequencing and/or two-step stem-loop real-time quantitative PCR (RT-qPCR) approaches. For the stem-loop RT-qPCR, total RNA was purified (miRvana, catalog #AM1560, ThermoFisher Scientific) from the same punch lysate used to analyze HTT mRNA and VG, and a stem-loop oligonucleotide homologous to the AAV1-VOYHT1 specific miRNA guide strand was used to prime the reverse transcriptase reaction to generate cDNA. Then, forward and reverse primers homologous to AAV1-VOYHT1 specific miRNA and the stem-loop were used for a traditional qPCR reaction (second) step. Both the stem-loop primer and the qPCR probe set were custom-designed for the specific detection of the AAV1-VOYHT1 miRNA guide strand. All samples were blinded during the analysis. Statistical comparison of the data was performed using the one-way ANOVA Tukey's multiple comparison test. A P value of less than 0.05 indicates a statistically significant difference.

For HTT mRNA knockdown, the relative HTT mRNA levels in all putamen punches from each AAV1-VOYHT1-treated group after normalization to the vehicle control group are presented in Table 60. Averages of 12%, 61% and 67% of HTT mRNA knockdown were achieved in putamen punches via bilateral thalamus only dosing (Group C2), and medium (Group C3) and high dose (Group C4) of bilateral putamen and thalamus dosing, respectively. Bilateral thalamus only dosing resulted in statistically significant HTT mRNA knockdown in the putamen. The percentage of punches that exhibited over 30% HTT knockdown in each group is also shown in Table 60. Both medium and high doses of bilateral putamen and thalamus dosing resulted in over 60% of putamen punches exhibiting over 30% HTT mRNA knockdown, with the high dose group having all punches exceeding 30% HTT mRNA knockdown.

TABLE 60
HTT knockdown in all putamen punches

| | | | HTT mRNA relative | P value by one-way | |
|---|---|---|---|---|---|
| | | Sample | to vehicle (% ± | ANOVA (vs | % Punches ≥ |
| Group | Description | size | stdev) | vehicle) | 30% KD |
| Group C1 | Vehicle | 48 | 100 ± 8 | — | 0 |
| Group C2 | Thalamus only | 48 | 88 ± 11 | <0.0001 | 6.3 |
| Group C3 | Medium dose | 48 | 39 ± 17 | <0.0001 | 89.6 |
| Group C4 | High dose | 48 | 33 ± 10 | <0.0001 | 100 |

The relative HTT mRNA levels analyzed from each animal in the AAV1-VOYHT1-treated groups after normalization to the vehicle control group are presented in Table 61.

TABLE 61
HTT knockdown in putamen punches averaged per animal

| | | | | | P value by | P value by |
|---|---|---|---|---|---|---|
| | | | | | one-way | one-way |
| | | | HTT mRNA | P value by one- | ANOVA (vs | ANOVA (vs |
| | | Sample | relative to vehicle | way ANOVA | Thalamus | Medium |
| Group | Description | size | (% ± stdev) | (vs vehicle) | only) | dose) |
| Group C1 | Vehicle | 3 | 100 ± 6 | — | — | — |
| Group C2 | Thalamus only | 3 | 88 ± 3 | 0.1178 | — | — |
| Group C3 | Medium dose | 3 | 39 ± 8 | <0.0001 | <0.0001 | — |
| Group C4 | High dose | 3 | 33 ± 4 | <0.0001 | <0.0001 | 0.6295 |

For VG levels, the average number of vector genome copies detected in all putamen punches from each group is presented in Table 62. Averages of 21, 869, and 1211 VG copies per diploid cell were achieved in the putamen punches via bilateral thalamus only, and medium and high dose of bilateral putamen and thalamus dosing, respectively. Both medium and high doses of bilateral putamen and thalamus dosing resulted in significantly higher VG distribution to the putamen than bilateral thalamus only dosing.

TABLE 62
VG copies in all putamen punches

| | | | VG copies/cell |
|---|---|---|---|
| Group | Description | Sample site | (mean ± stdev) |
| Group C1 | Vehicle | 48 | 0.43 ± 1.33 |
| Group C2 | Thalamus only | 48 | 21.03 ± 9.05 |
| Group C3 | Medium dose | 48 | 869.3 ± 846.6 |
| Group C4 | High dose | 48 | 1211 ± 1047.0 |

The number of vector genome copies analyzed from each animal is presented in Table 63.

TABLE 63
VG copies in putamen punches averaged per animal

| | | | VG copies/cell |
|---|---|---|---|
| Group | Description | Sample size | (mean ± stdev) |
| Group C1 | Vehicle | 3 | 0.39 ± 0.53 |
| Group C2 | Thalamus only | 3 | 21.03 ± 3.72 |
| Group C3 | Medium dose | 3 | 869.3 ± 338.0 |
| Group C4 | High dose | 3 | 1211 ± 540.1 |

A Grubbs' test (Q=0.1%) was applied for removal of outliers and the VG copies/cell recalculated. Following this post-hoc statistical analysis, VG copies in putamen punches per animal were quantified as 21.0±6.5, 869.3±283.0 and 1210.8±387.3 for groups C2, C3 and C4, respectively.

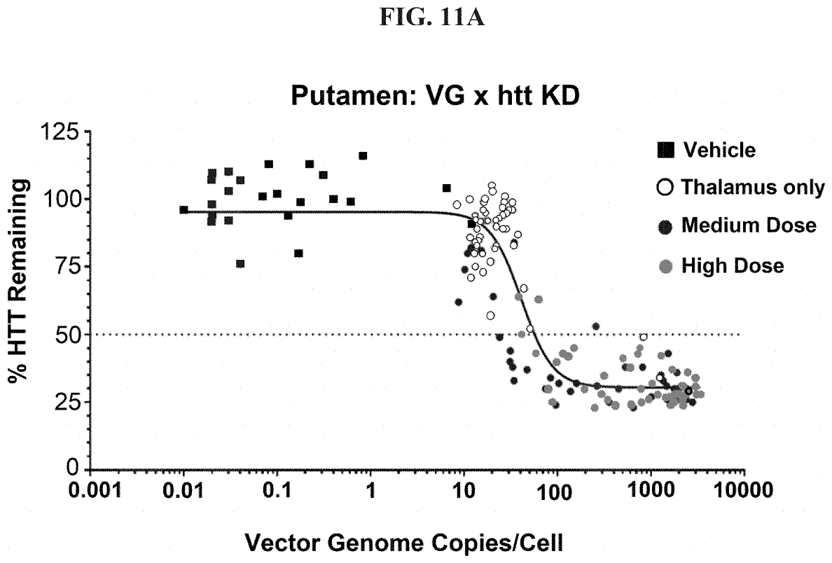

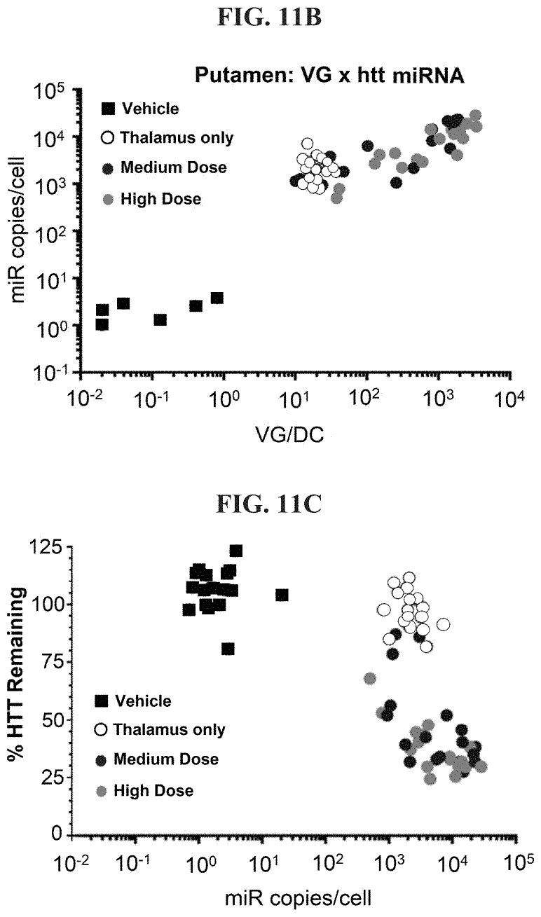

The correlation of HTT mRNA knockdown versus vector genome levels in the putamen punches is shown in FIG. 11A. The correlation curve of all putamen punches from all dosing groups results in a dose-response curve with the vehicle group at the top, thalamus only group predominantly at the top shoulder, medium dose group evenly distributed along the slope and the base, and the high dose group predominantly at the base of the curve. The EC₅₀ for HTT knockdown was calculated (Graphpad Prism, nonlinear regression 4 parameter curve fit) at approximately 40 VG per diploid cell (the range is 20-50 VG per diploid cell).

For miRNA analyses, the average number of AAV1-VOYHT1 specific miRNA copies per cell and corresponding average VG copies per cell, HTT mRNA levels relative to control, and AAV1-VOYHT1 specific miRNA per VG calculation averaged per animal are presented in Table 64. These analyses were performed using a subset of putamen punches, and thus, values presented in Table 64 refer to data from 6 putamen punches per animal (3 per hemisphere) for a total of 72 total samples.

TABLE 64
AAV1-VOYHT1 specific mRNA expression in putamen punches averaged
per animal

| | | | miRNA | VG copies/ | HTT mRNA | |
|---|---|---|---|---|---|---|
| | | | copies/cell | cell | relative to | HTT |
| | | Sample | (mean ± | (mean ± | vehicle (% ± | miRN/VG |
| Group | Description | size | stdev) | stdev) | stdev) | (mean ± stdev) |
| Group C1 | Vehicle | 3 | 2.9 ± 4.44 | 0.08 ± 0.21 | 100 ± 9 | 15 ± 32 |
| Group C2 | Thalamus only | 3 | 2283 ± 1495 | 21 ± 7 | 91 ± 8 | 128 ± 107 |
| Group C3 | Medium dose | 3 | 8808 ± 7955 | 793 ± 773 | 44 ± 18 | 44 ± 55 |
| Group C4 | High dose | 3 | 8869 ± 7568 | 1258 ± 1118 | 35 ± 10 | 11 ± 7 |

The correlation of AAV1-VOYHT1 specific miRNA expression versus vector genome levels in all putamen punches from each treatment group (r=0.8606, p<0.001)) is shown in FIG. 11B. Enhanced VG biodistribution corresponded to increased AAV1-VOYHT1 specific miRNA expression in AAV1-VOYHT1-treated group.

The correlation of AAV1-VOYHT1 specific miRNA expression versus vector HTT mRNA lowering in all putamen punches from each treatment group (r=−0.6788, p<0.0001) is shown in FIG. 11C. Increased AAV1-VOYHT1 specific miRNA expression corresponded to enhanced HTT mRNA lowering AAV1-VOYHT1-treated group.

Together, thalamus-only dosing resulted in more modest VG biodistribution, AAV1-VOYHT1 specific miRNA expression, and HTT mRNA lowering in putamen. Combined putamen and thalamus dosing resulted in greater VG biodistribution, AAV1-VOYHT1 specific miRNA expression, and robust HTT mRNA lowering in putamen relative to thalamus only dosing. Finally, AAV1-VOYHT1 specific miRNA expression correlates with VG biodistribution and HTT mRNA lowering.

xii. HTT Knockdown. VG Measurement and AAV1-VOYHT1 Specific miRNA Expression in Punches from NHP Caudate

A selected brain slab containing the caudate from all four groups was used to collect 2 mm caudate punches. Two punches were collected from each side of the slab, with a total of 4 punches collected from each animal. A total of 48 caudate punches was collected from all 12 animals. Samples were processed and analyzed for HTT mRNA levels and VG levels using bDNA and ddPCR, respectively. bDNA and ddPCR were carried out as described in Example 18. Samples were processed and analyzed for AAV1-VOYHT1 specific miRNA levels using deep sequencing and/or two-step stem-loop real-time quantitative PCR (RT-qPCR) approaches. For the stem-loop RT-qPCR, total RNA was purified (miRvana, catalog #AM1560, ThermoFisher Scientific) from the same punch lysate used to analyze HTT mRNA and VG, and a stem-loop oligonucleotide homologous to the AAV1-VOYHT1 specific miRNA guide strand was used to prime the reverse transcriptase reaction to generate cDNA. Then, forward and reverse primers homologous to AAV1-VOYHT1 specific miRNA and the stem-loop were used for a traditional qPCR reaction (second) step. Both the stem-loop primer and the qPCR probe set were custom-designed for the specific detection of the AAV1-VOYHT1 miRNA guide strand. All samples were blinded during the analysis. Statistical comparison of the data was performed using the one-way ANOVA Tukey's multiple comparison test. A P value of less than 0.05 indicates a statistically significant difference.

For HTT mRNA knockdown, the relative HTT mRNA levels in all caudate punches from each AAV1-VOYHT1-treated group after normalization to the vehicle control group are presented in Table 63. Averages of 51%, 61% and 68% of HIT mRNA knockdown were achieved in caudate punches via bilateral thalamus only dosing (Group C2), and medium (Group C3) and high dose (Group C4) of bilateral putamen and thalamus dosing, respectively. Bilateral thalamus only dosing caused robust and significant HTT mRNA knockdown (by 51%) in the caudate punches. The percentage of punches that exhibited over 30% HTT knockdown in each group is also shown in Table 65. All three dosing groups (bilateral thalamus only dosing, medium and high dose of bilateral putamen and thalamus dosing) had 92% of caudate punches achieving at least 30% HTT mRNA knockdown.

TABLE 65
HTT knockdown in all caudate punches

| | | Sample | HTT mRNA Relative | P value by one-way | % Punches ≥ |
|---|---|---|---|---|---|
| Group | Description | size | to Vehicle (% ± stdev) | ANOVA (vs vehicle) | 30% KD |
| Group C1 | Vehicle | 12 | 100 ± 6 | — | 0 |
| Group C2 | Thalamus only | 12 | 49 ± 16 | <0.0001 | 92 |
| Group C3 | Medium dose | 12 | 39 ± 15 | <0.0001 | 92 |
| Group C4 | High dose | 12 | 32 ± 15 | <0.0001 | 92 |

The relative HTT mRNA levels analyzed from each animal in the AAV1-VOYHT1-treated groups after normalization to the vehicle control group are presented in Table 66.

TABLE 66
HTT knockdown in caudate punches averaged per animal

| | | | HTT mRNA | P value by | P value by one | P value by |
|---|---|---|---|---|---|---|
| | | | Relative to | one-way | way ANOVA | one-way |
| | | Sample | Vehicle (% ± | ANOVA (vs | (vs Thalamus | ANOVA (vs |
| Group | Description | size | stdev) | vehicle) | only) | Medium dose) |
| Group | Vehicle | 3 | 100 ± 6 | — | — | — |
| C1 | | | | | | |
| Group | Thalamus only | 3 | 49 ± 13 | 0.0012 | — | — |
| C2 | | | | | | |
| Group | Medium dose | 3 | 39 ± 11 | 0.0004 | 0.6889 | — |
| C3 | | | | | | |
| Group | High dose | 3 | 32 ± 9 | 0.0002 | 0.9786 | 0.8347 |
| C4 | | | | | | |

For VG levels, the average number of vector genome copies detected in all caudate punches from each group is presented in Table 67. An average of 44, 146, and 99 vector genome copies per diploid cell was achieved in the caudate punches via bilateral thalamus-only dosing, medium and high doses of bilateral putamen and thalamus dosing, respectively.

TABLE 67
VG copies in all caudate punches

| | | | VG copies/cell |
|---|---|---|---|
| Group | Description | Sample size | (mean ± stdev) |
| Group C1 | Vehicle | 12 | 0.12 ± 0.15 |
| Group C2 | Thalamus only | 12 | 44.18 ± 22.86 |
| Group C3 | Medium dose | 12 | 146.2 ± 200.9 |
| Group C4 | High dose | 12 | 99.22 ± 45.01 |

The number of vector genome copies analyzed from each animal is presented in Table 68.

TABLE 68
VG copies in caudate punches per animal

| | | | VG copies/cell |
|---|---|---|---|
| Group | Description | Sample size | (mean ± stdev) |
| Group C1 | Vehicle | 3 | 0.10 ± 0.05 |
| Group C2 | Thalamus only | 3 | 44.18 ± 10.16 |
| Group C3 | Medium dose | 3 | 146.2 ± 182.9 |
| Group C4 | High dose | 3 | 99.22 ± 29.22 |

A Grubbs' test (Q=0.1%) was applied for removal of outliers and the VG copies/cell in caudate recalculated. Following this post-hoc statistical analysis, VG copies in caudate punches per animal were quantified as 44.2±10.2, 107.4±116.0 and 99.2±29.2 for groups C2, C3 and C4, respectively.

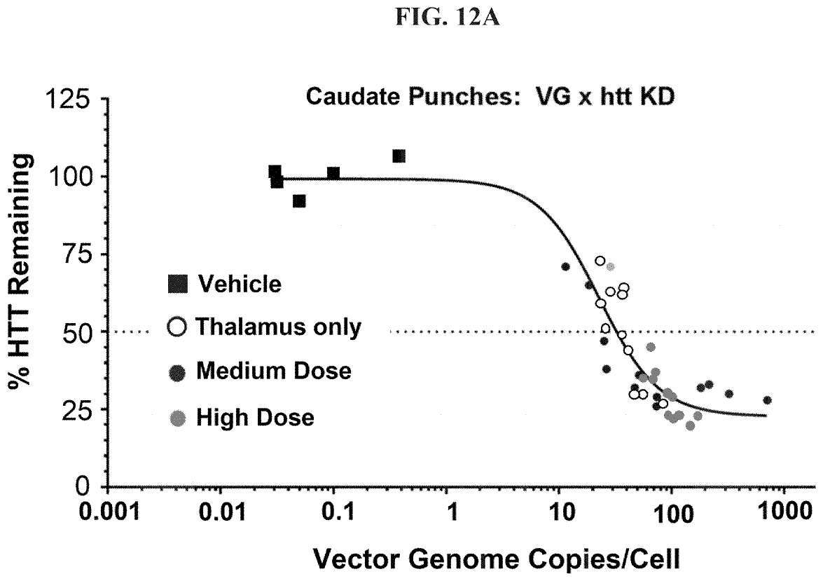

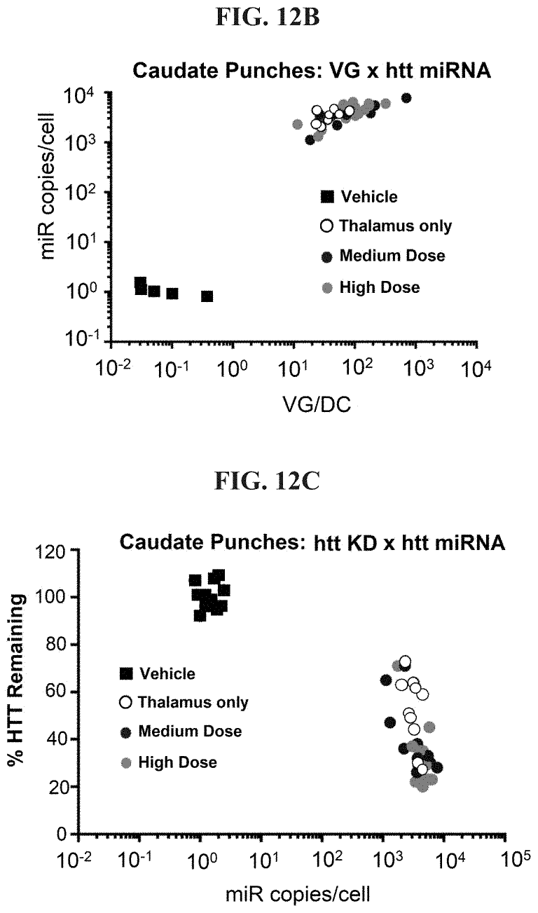

The correlation of HTT mRNA knockdown versus vector genome levels in the caudate punches is shown in FIG. 12A. The correlation curve of all caudate punches from all dosing groups results in a dose-response curve with the vehicle group at the top, and all other dosing groups dispersed along the slope and beginning of the base of the curve. The EC₅₀ for HTT knockdown was calculated (Graphpad Prism, nonlinear regression 4 parameter curve fit) at approximately 23 VG per diploid cell (the range is 20-50 VG per diploid cell).

For miRNA analyses, the average number of AAV1-VOYHT1 specific miRNA copies per cell and corresponding average VG copies per cell, HTT mRNA levels relative to control, and AAV1-VOYHT1 specific miRNA per VG calculation averaged per animal are presented in Table 69. These analyses were performed using a subset of caudate punches, and thus, values presented in Table 69 refer to data from 4 putamen punches per animal (2 per hemisphere) for a total of 48 total samples.

TABLE 69
AAV1-VOYHT1 specific miRNA expression in caudate punches averaged per
animal

| | | | | VG copies/ | HTT mRNA | |
|---|---|---|---|---|---|---|
| | | | miRNA | cell | relative to | HTT |
| | | Sample | copies/cell | (mean ± | vehicle (% ± | miRNA/VG |
| Group | Description | size | (mean ± stdev) | stdev) | stdev) | (mean ± stdev) |
| Group C1 | Vehicle | 3 | 1.5 ± 0.53 | 0.05 ± 0.11 | 100 ± 6 | 10 ± 19 |
| Group C2 | Thalamus only | 3 | 3535 ± 1050 | 44 ± 10 | 49 ± 13 | 90 ± 35 |
| Group C3 | Medium dose | 3 | 3730 ± 1944 | 146 ± 183 | 39 ± 11 | 62 ± 55 |
| Group C4 | High dose | 3 | 4468 ± 1356 | 99 ± 29 | 32 ± 9 | 51 ± 20 |

The correlation of AAV1-VOYHT1 specific miRNA expression versus vector genome levels in all caudate punches from each treatment group (r=0.6782, p<0.0001) is shown in FIG. 12B. Enhanced VG biodistribution corresponded to increased AAV1-VOYHT1 specific miRNA expression in AAV1-VOYHT1-treated group. A Grubbs' test (Q=0.1%) was used to detect significant outliers when vector genome level data from animal groups in Dose Optimization Study III were combined with those from Dose Optimization Study I, as above. A positive relationship between AAV1-VOYHT1 specific miRNA expression versus vector genome levels in caudate punches from each treatment group remained following the removal of one outlier value from the correlation analysis shown in FIG. 12B (r=0.7452, p<0.001). Following removal of all outliers, VG copies/cell (mean±stdev) in caudate punches for Groups A2, A3, A5 and C3 to 1.8±0.5, 10.7±10.3, 0.3±0.2 and 107.4±116.0 VG copies/cell, respectively.

The correlation of AAV1-VOYHT1 specific miRNA expression versus vector HTT mRNA lowering in all caudate punches from each treatment group (r=−0.8798, p<0.0001) is shown in FIG. 12C. Increasing AAV1-VOYHT1 specific miRNA expression corresponded to enhanced HTT mRNA lowering AAV1-VOYHT1-treated group.

Together, thalamus-only dosing resulted in significant VG biodistribution, significant AAV1-VOYHT1 specific miRNA expression, and substantial HTT mRNA lowering in caudate. Combined putamen and thalamus dosing resulted in greater VG biodistribution, AAV1-VOYHT1 specific miRNA expression, and robust HTT mRNA lowering in caudate compared with thalamus-only dosing. Finally, AAV1-VOYHT1 specific miRNA expression correlates with VG biodistribution and HTT mRNA lowering.

xiii. HTT Knockdown and VG Measurement in Punches from NHP Thalamus

A selected brain slab containing the thalamus from all four groups was used to collect 2 mm thalamus punches. Five punches were collected from each side of the slab, with a total of 10 punches collected from each animal. A total number of 120 thalamus punches were collected from all 12 animals. Samples were processed and analyzed for HTT mRNA levels and VG levels using bDNA and ddPCR, respectively. bDNA and ddPCR were carried out as described in Example 18. All samples were blinded during the analysis. Statistical comparison of the data was performed using the one-way ANOVA Tukey's multiple comparison test. A P value of less than 0.05 indicates a statistically significant difference.

For HTT mRNA knockdown, the relative HTT mRNA levels in all thalamus punches from the AAV1-VOYHT1-treated groups after normalization to the vehicle control group are presented in Table 70. Averages of 76%, 76% and 73% of HTT mRNA knockdown were achieved in the thalamus punches via bilateral thalamus only dosing (Group C2), and medium (Group C3) and high dose (Group C4) of bilateral putamen and thalamus dosing, respectively. The percentage of punches that exhibited over 30% HTT knockdown in each group is also shown in Table 70. 100% of the thalamus punches achieved at least 30% of HTT mRNA KD for all three dosing groups.

TABLE 70
HTT knockdown in all thalamus punches

| | | Sample | HTT mRNA relative | P value by one-way | % Punches ≥ |
|---|---|---|---|---|---|
| Group | Description | size | to vehicle (% ± stdev) | ANOVA (vs vehicle) | 30% KD |
| Group C1 | Vehicle | 30 | 100 ± 8 | — | 0 |
| Group C2 | Thalamus only | 30 | 24 ± 5 | <0.0001 | 100 |
| Group C3 | Medium dose | 30 | 24 ± 9 | <0.0001 | 100 |
| Group C4 | High dose | 30 | 27 ± 5 | <0.0001 | 100 |

The relative HTT mRNA levels analyzed from each animal in the AAV1-VOYHT1-treated groups after normalization to the vehicle control group are presented in Table 71.

TABLE 71
HTT knockdown in thalamus punches averaged per animal

| | | | HTT mRNA | P value by | P value by one | P value by |
|---|---|---|---|---|---|---|
| | | | Relative to | one-way | way ANOVA | one-way |
| | | Sample | Vehicle (% ± | ANOVA (vs | (vs Thalamus | ANOVA (vs |
| Group | Description | size | stdev) | vehicle) | only) | Medium dose) |
| Group | Vehicle | 3 | 100 ± 3 | — | — | — |
| C1 | | | | | | |
| Group | Thalamus only | 3 | 24 ± 4 | <0.0001 | — | — |
| C2 | | | | | | |
| Group | Medium dose | 3 | 24 ± 5 | <0.0001 | 0.9995 | — |
| C3 | | | | | | |
| Group | High dose | 3 | 27 ± 2 | <0.0001 | 0.7505 | 0.8081 |
| C4 | | | | | | |

For VG levels, the average number of vector genome copies detected in all thalamus punches from each group is presented in Table 72. Similar levels of vector genome copies in all 3 treatment groups were observed. Averages of 2015, 1704, 2747 vector genome copies per diploid cell were achieved in the thalamus punches via bilateral thalamus-only dosing, medium and high dose of bilateral putamen and thalamus dosing, respectively.

TABLE 72
VG copies in all thalamus punches

| | | | VG copies/cell |
|---|---|---|---|
| Group | Description | Sample size | (mean ± stdev) |
| Group C1 | Vehicle | 30 | 0.13 ± 0.27 |
| Group C2 | Thalamus only | 30 | 2015 ± 1088 |
| Group C3 | Medium dose | 30 | 1704 ± 741.9 |
| Group C4 | High dose | 30 | 2747 ± 896.1 |

The number of vector genome copies analyzed from each animal is presented in Table 73.

TABLE 73
VG copies in thalamus punches averaged per animal

| | | | VG copies/cell |
|---|---|---|---|
| Group | Description | Sample size | (mean ± stdev) |
| Group C1 | Vehicle | 3 | 0.13 ± 0.07 |
| Group C2 | Thalamus only | 3 | 2015 ± 310.9 |
| Group C3 | Medium dose | 3 | 1704 ± 467.3 |
| Group C4 | High dose | 3 | 2747 ± 691.2 |

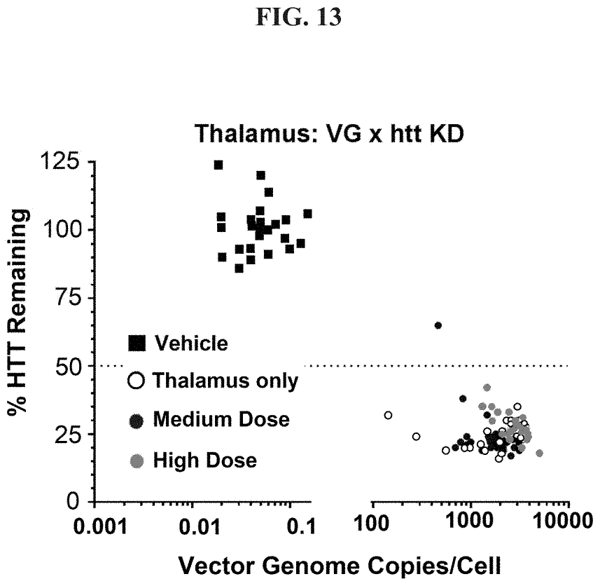

The correlation of HTT mRNA knockdown versus vector genome levels in the thalamus punches is shown in FIG. 13. All dosing groups achieved similar vector genome copies per cell and similar knockdown efficiency in punches from the thalamus. The correlation plot of all thalamus punches from all dosing groups shows the vehicle group in the top left and all other dosing groups mostly overlapping one another in the far right of the base. The EC₅₀ calculations were ambiguous due to the presence of predominantly fully positive and negative populations.

In summary, the punch analyses from the putamen, caudate, and thalamus reveal that substantial HTT mRNA knockdown was achieved at the infusion sites (putamen and thalamus) as well as in the caudate in all three dosing groups (thalamus-only dosing, and medium and high doses of bilateral putamen and thalamus dosing). Further, vector genome levels correlate well with HTT mRNA knockdown in the putamen, caudate and thalamus with evidence for a plateau in knockdown at high vector genome levels.

xiv. Clinical Signs and Histopathology

In 7 out of 9 NHPs that received AAV1-VOYHT1, no clinical signs or limb findings were observed post-infusion. In the other two NHPs, shortened steps and slight limb finding were observed. However, no histopathological changes were seen which would account for, or correlate with these clinical signs. Histopathologic findings associated with catheter tip and/or track were expected due to the surgical procedure, but none resulted in any specific clinical sign. Minimal findings at the thalamic sites of infusion were expected and included gliosis, neuronal degeneration, glial cell vacuolation and mononuclear cell infiltration that were slightly more wide-spread than in putamen. None was expected to result in any clinical signs. Edema was only observed adjacent to the catheter track, suggesting that volumes were well-tolerated. No evidence of detrimental effect on neurons of the somatosensory or motor cortices was seen in any group. These findings suggest that the no-observed-adverse-effect-level (NOAEL) is, at a minimum, AAV1-VOYHT1 administered at the high dose via putamen and thalamus infusion (see Group C4).

### Example 21. Formulation Optimization

Initial formulation screening identified a Phosphate/Sucrose/NaCl formulation (2.7 mM Na Phosphate (dibasic), 1.54 mM K Phosphate (mono), 155 mM NaCl, and 5% (w/v) Sucrose at pH 7.2, 450 mOsm/kg) as an acceptably stable formulation for the AAV1-VOYHT1 vector. High salt formulations were also identified as stabilizing.

The formulation was further optimized for excipients, Na/K ratios, pH, and osmolality while adjusting for factors suitable for CNS administration. Three solutions that may be used to formulate the AAV1-VOYHT1 vector are presented in Table 74.

TABLE 74
Formulations for AAV1-VOYHT1 vector

| Formulation 1 | Formulation 2 | Formulation 3 |
|---|---|---|
| 10 mM Sodium Phosphate | 10 mM Tris Base | 10 mM Tris Base |
| 1.5 mM Potassium Phosphate | 6.25 mM HCl | 8.95 mM HCl |
| 95 mM Sodium Chloride | 1.5 mM Potassium Chloride | 1.5 mM Potassium Chloride |
| 7% (w/v) Sucrose | 100 mM Sodium Chloride | 100 mM Sodium Chloride |
| 0.001% (w/v) Pluronic ® F-68 | 7% (w/v) Sucrose | 7% (w/v) Sucrose |
| pH 7.4 ± 0.2 at 5° C. | 0.001% (w/v) Pluronic ® F-68 | 0.001% (w/v) Pluronic ® F-68 |
| | pH 8.0 ± 0.2 at 5° C. | pH 7.5 ± 0.2 at 5° C. |

The concentration of the AAV1-VOYHT1 vector to be formulated in the above identified solutions is about 2.7e13 vg/mL, but the concentration may be increased up to 5e13 vg/ml. High concentration AAV1-VOYHT1 vectors were shown to be difficult to stabilize in the absence of aggregation. Analysis of a formulation screen indicated that an increase in sucrose level generally improves vector stability and prevents aggregation. Sucrose levels from about 5% to 9% provided good stability for the AAV1-VOYHT1 vector, with the optimal concentration at about 7% sucrose for the tested vector and desired formulation concentration. The level of sucrose use may be limited by physiological osmolality. Furthermore, higher osmolality and/or more NaCl were shown to be favorable for vector stability.

### Example 22. Administration of AAV1-VOYHT1 to HD Patients

AAV1-VOYHT1 vectors formulated in an appropriate formulation identified in Example 21 are administered into a Stage 1 HD patient via bilateral parenchymal infusion to the putamen and thalamus using MRI-guided convection enhanced delivery (CED). The concentration of AAV1-VOYHT1 vectors in the formulated solution to be infused is between 2.7e12 to 2.7e13 vg/mL. The volumes of AAV1-VOYHT1 infused to the putamen and thalamus are 300-1500 μL/hemisphere and 1300-2500 μL/hemisphere, respectively. The doses administered to the putamen and the thalamus are 8e11 to 4e13 vg/hemisphere and 3.5e12 to 6.8e13 vg/hemisphere, respectively. The total dose administered to the patient is about 8.6e12 to 2c14 vg. The AAV1-VOYHT1 treatment results in significant reduction in HTT mRNA levels in the striatum and cortex of the patient.

We claim:

1. A method of producing a pharmaceutical formulation comprising adeno-associated virus (AAV) particles, said method comprising:
Producing AAV particles in one or more viral production cells (VPCs) within a bioreactor, thereby providing a viral production pool which comprises the AAV particles and a liquid media;
Processing the viral production pool through one or more steps selected from: chemical lysis, clarification filtration, affinity chromatography, ion-exchange chromatography, tangential flow filtration (TFF), and virus retentive filtration; and
Incorporating the AAV particles from the viral production pool into a pharmaceutical formulation, wherein the pharmaceutical formulation comprises the AAV particles and at least one pharmaceutical excipient.

2. The method of claim 1, wherein the VPCs comprise Sf9 insect cells, and wherein the AAV particles are produced using a baculovirus production system.

3. The method of claim 1 or claim 2, wherein the method comprises:
Collecting the viral production pool from the bioreactor, wherein the viral production pool comprises the one or more VPCs, and wherein the AAV particles are contained within the VPCs; and
Exposing the VPCs within the viral production pool to chemical lysis using a chemical lysis solution under chemical lysis conditions, wherein the chemical lysis releases the AAV particles from the VPCs into the liquid media of the viral production pool.

4. The method of claim 3, wherein the chemical lysis solution comprises a stabilizing additive selected from arginine or arginine salts.

5. The method of claim 4, wherein the concentration of the stabilizing additive is between 0.1-0.5 M.

6. The method of claim 4, wherein the concentration of the stabilizing additive is between 0.2-0.3 M.

7. The method of any one of claims 3-6, wherein the chemical lysis solution does not include Triton X-100.

8. The method of any one of claims 3-7, wherein the chemical lysis solution comprises a zwitterionic detergent selected from Lauryl dimethylamine N-oxide (LDAO); N,N-Dimethyl-N-dodecylglycine betaine (Empigen BB); 3-(N,N-Dimethyl myristylammonio) propanesulfonate (Zwittergent 3-10); n-Dodecyl-N,N-dimethyl-3-ammonio-1-propanesulfonate (Zwittergent 3-12); n-Tetradecyl-N,N-dimethyl-3-ammonio-1-propanesulfonate (Zwittergent 3-14); 3-(N,N-Dimethyl palmitylammonio) propanesulfonate (Zwittergent 3-16); 3-((3-cholamidopropyl) dimethylammonio)-1-propanesulfonate (CHAPS); or 3-([3-Cholamidopropyl]dimethylammonio)-2-hydroxy-1-propanesulfonate (CHAPSO).

9. The method of any one of claims 3-7, wherein the chemical lysis solution comprises Lauryl dimethylamine N-oxide (LDAO).

10. The method of any one of claims 3-7, wherein the chemical lysis solution comprises N,N-Dimethyl-N-dodecylglycine betaine (Empigen BB).

11. The method of any one of claims 1-10, wherein the method comprises one or more clarification filtration steps in which the viral production pool is processed through one or more clarification filtration systems.

12. The method of claim 11, wherein the one or more clarification filtration systems comprises a depth filtration system.

13. The method of claim 12, wherein the depth filtration system comprises a Millipore Millistak D0HC media series filter.

14. The method of claim 12, wherein the depth filtration system comprises a Millipore Millistak C0SP media series filter.

15. The method of any one of claims 11-14, wherein the one or more clarification filtration systems comprises a 0.2 μm microfiltration system.

16. The method of any one of claims 1-15, wherein the method comprises one or more affinity chromatography steps in which the viral production pool is processed through one or more affinity chromatography systems.

17. The method of claim 16, wherein the method comprises processing the viral production pool through one or more immunoaffinity chromatography systems in bind-elute mode; wherein the immunoaffinity chromatography system comprises one or more recombinant single-chain antibodies which are capable of binding to one or more AAV capsid variants.

18. The method of claim 16 or claim 17, wherein the immunoaffinity chromatography system is regenerated using a regeneration solution, wherein the regeneration solution comprises between 1-3 M of guanidine or a guanidine salt.

19. The method of claim 16 or claim 17, wherein the immunoaffinity chromatography system is regenerated using a regeneration solution which comprises 2 M guanidine HCL.

20. The method of any one of claims 1-19, wherein the method comprises one or more ion exchange chromatography steps in which the viral production pool is processed through one or more ion exchange chromatography systems.

21. The method of claim 20, wherein the method comprises processing the viral production pool through one or more anion exchange chromatography systems in flow-through mode; wherein the anion exchange chromatography system comprises a stationary phase which binds non-viral impurities, non-AAV viral particles, or a combination thereof; and wherein the stationary phase of the anion exchange chromatography system does not bind to AAV particles.

22. The method of claim 21, wherein the stationary phase of the anion exchange chromatography system comprises a quaternary amine functional group.

23. The method of claim 21, wherein the stationary phase of the anion exchange chromatography system comprises a trimethylammonium ethyl (TMAE) functional group.

24. The method of any one of claims 1-23, wherein the method comprises one or more tangential flow filtration (TFF) steps in which the viral production pool is processed through one or more tangential flow filtration (TFF) systems.

25. The method of claim 24, wherein the TFF system comprises a flat-sheet filter comprising a regenerated cellulose cassette.

26. The method of claim 25, wherein the TFF system is operated at a transmembrane pressure (TMP) of between 5.5-6.5 PSI, and a target crossflow between 5.5-6.5 L/min/m².

27. The method of any one of claims 24-26, wherein a 50% sucrose mixture is added to the viral production pool prior to the one or more TFF steps; and wherein the 50% sucrose mixture is added to the viral production pool at a centration between 9-13% v/v.

28. The method of any one of claims 24-27, wherein the one or more TFF steps comprises a first diafiltration step in which at least a portion of the liquid media of the viral production pool is replaced with a low-sucrose diafiltration buffer, wherein the low-sucrose diafiltration buffer comprises between 4-6% w/v of a sugar or sugar substitute and between 150-250 mM of an alkali chloride salt.

29. The method of claim 28, wherein the low-sucrose diafiltration buffer comprises between 4.5-5.5% w/v of sucrose and between 210-230 mM sodium chloride.

30. The method of claim 28, wherein the low-sucrose diafiltration buffer comprises 5% w/v of sucrose and 220 mM sodium chloride.

31. The method of any one of claims 24-30, wherein the one or more TFF steps comprises an ultrafiltration concentration step, wherein the AAV particles in the viral production pool are concentrated to between 1.0×10¹²-5.0×10¹³ vg/mL.

32. The method of claim 31, wherein the AAV particles in the viral production pool are concentrated to between 1.0-5.0×10¹³ vg/mL.

33. The method of claim 31, wherein the AAV particles in the viral production pool are concentrated to 2.7×10¹³ vg/mL.

34. The method of any one of claims 24-33, wherein the one or more TFF steps comprises a final diafiltration step in which at least a portion of the liquid media of the viral production pool is replaced with a high-sucrose formulation buffer, wherein the high-sucrose formulation buffer comprises between 6-8% w/v of a sugar or sugar substitute and between 90-100 mM of an alkali chloride salt.

35. The method of claim 34, wherein the high-sucrose formulation buffer comprises 7% w/v of sucrose and between 90-100 mM sodium chloride.

36. The method of claim 34, wherein the high-sucrose formulation buffer comprises 7% w/v of sucrose, 10 mM Sodium Phosphate, between 95-100 mM sodium chloride, and 0.001% (w/v) Poloxamer 188.

37. The method of any one of claims 1-36, wherein the method comprises one or more virus retentive filtration (VRF) steps in which the viral production pool is processed through one or more virus retentive filtration (VRF) systems.

38. The method of claim 37, wherein the VRF system comprises a filter medium which retains particles which are 35 nm or larger.

39. The method of claim 37, wherein the VRF system comprises a filter medium which retains particles which are 20 nm or larger.

40. A method of producing a gene therapy product, comprising: (i) providing a pharmaceutical formulation comprising AAV particles, wherein the pharmaceutical formulation is produced by the method of any one of claims 1-39; and (ii) suitably aliquoting the pharmaceutical formulation into a formulation container.

41. A pharmaceutical formulation comprising: (i) AAV particles at a concentration less than 5×10¹³ vg/ml; (ii) one or more salts; (iii) one or more sugars or sugar substitutes; and (iv) one or more buffering agents; wherein the pharmaceutical formulation is an aqueous formulation.

42. The pharmaceutical formulation of claim 41, wherein the pharmaceutical formulation comprises AAV particles at a concentration between 1.0×10¹²-5.0×10¹³ vg/mL.

43. The pharmaceutical formulation of claim 41, wherein the pharmaceutical formulation comprises AAV particles at a concentration between 1.0-5.0×10¹³ vg/mL.

44. The pharmaceutical formulation of claim 41, wherein the pharmaceutical formulation comprises AAV particles at a concentration between 2.7×10¹³ vg/mL.

45. The pharmaceutical formulation of any one of claims 41-44, wherein the one or more salts of the formulation comprises sodium chloride.

46. The pharmaceutical formulation of claim 45, wherein the concentration of sodium chloride in the formulation is between 80-220 mM.

47. The pharmaceutical formulation of claim 45, wherein the concentration of sodium chloride in the formulation is between 85-110 mM.

48. The pharmaceutical formulation of claim 45, wherein the concentration of sodium chloride in the formulation is 95 mM.

49. The pharmaceutical formulation of claim 45, wherein the concentration of sodium chloride in the formulation is between 100 mM.

50. The pharmaceutical formulation of any one of claims 41-49, wherein the one or more salts of the formulation comprises potassium chloride.

51. The pharmaceutical formulation of claim 50, wherein the concentration of potassium chloride in the formulation is between 0-10 mM.

52. The pharmaceutical formulation of claim 50, wherein the concentration of potassium chloride in the formulation is between 1-3 mM.

53. The pharmaceutical formulation of claim 50, wherein the concentration of potassium chloride in the formulation is between 1-2 mM.

54. The pharmaceutical formulation of claim 50, wherein the concentration of potassium chloride in the formulation is 1.5 mM.

55. The pharmaceutical formulation of any one of claims 41-54, wherein the one or more salts of the formulation comprises potassium phosphate.

56. The pharmaceutical formulation of claim 55, wherein the concentration of potassium phosphate in the formulation is between 0-10 mM.

57. The pharmaceutical formulation of claim 55, wherein the concentration of potassium phosphate in the formulation is between 1-3 mM.

58. The pharmaceutical formulation of claim 55, wherein the concentration of potassium phosphate in the formulation is 1.5 mM.

59. The pharmaceutical formulation of any one of claims 41-58, wherein the concentration of the sugar or sugar substitute in the formulation is between 1-10% w/v.

60. The pharmaceutical formulation of any one of claims 41-58, wherein the concentration of the sugar or sugar substitute in the formulation is between 4-6% w/v.

61. The pharmaceutical formulation of any one of claims 41-58, wherein the concentration of the sugar or sugar substitute in the formulation is 5% w/v.

62. The pharmaceutical formulation of any one of claims 41-58, wherein the concentration of the sugar or sugar substitute in the formulation is between 6-8% w/v.

63. The pharmaceutical formulation of any one of claims 41-58, wherein the concentration of the sugar or sugar substitute in the formulation is 7% w/v.

64. The pharmaceutical formulation of any one of claims 41-63, wherein the one or more sugars or sugar substitutes comprises at least one disaccharide selected from sucrose, lactulose, lactose, maltose, trehalose, cellobiose, chitobiose, kojibiose, nigerose, isomaltose, β,β-trehalose, α,β-trehalose, sophorose, laminaribiose, gentiobiose, turanose, maltulose, palatinose, gentiobiulose, mannobiose, melibiose, melibiulose, rutinose, rutinulose, and xylobiose.

65. The pharmaceutical formulation of any one of claims 41-63, wherein the one or more sugars or sugar substitutes comprises sucrose.

66. The pharmaceutical formulation of any one of claims 41-63, wherein the one or more sugars or sugar substitutes comprises trehalose.

67. The pharmaceutical formulation of any one of claims 41-63, wherein the one or more sugars or sugar substitutes comprises sorbitol.

68. The pharmaceutical formulation of any one of claims 41-67, wherein the one or more buffering agents provide a formulation pH from 7.0 to 8.2 at 5° C.

69. The pharmaceutical formulation of any one of claims 41-68, wherein the buffering agent is at a concentration of 1-20 mM in the formulation.

70. The pharmaceutical formulation of any one of claims 41-68, wherein the buffering agent is at a concentration of 10 mM in the formulation.

71. The pharmaceutical formulation of any one of claims 41-70, wherein the one or more buffering agents is selected from Tris HCl, Tris base, sodium phosphate, potassium phosphate, histidine, boric acid, citric acid, glycine, HEPES (4-(2-hydroxyethyl)-1-piperazineethanesulfonic acid), and MOPS (3-(N-morpholino)propanesulfonic acid).

72. The pharmaceutical formulation of any one of claims 41-70, wherein the one or more buffering agents comprises sodium phosphate and the formulation pH is from 7.2 to 7.6 at 5° C.

73. The pharmaceutical formulation of claim 72, wherein the concentration of the sodium phosphate in the formulation is between 8-11 mM.

74. The pharmaceutical formulation of claim 72, wherein the concentration of the sodium phosphate in the formulation is 10 mM.

75. The pharmaceutical formulation of any one of claims 41-70, wherein the one or more buffering agents comprises Tris base adjusted with hydrochloric acid, and the formulation pH is from 7.8 to 8.2 at 5° C.

76. The pharmaceutical formulation of any one of claims 41-70, wherein the one or more buffering agents comprises Tris base adjusted with hydrochloric acid, and the formulation pH is from 7.3 to 7.7 at 5° C.

77. The pharmaceutical formulation of any one of claims 41-76, wherein the pharmaceutical formulation comprises a copolymer surfactant.

78. The pharmaceutical formulation of claim 77, wherein the concentration of the copolymer surfactant is between 0.00001%-1% w/v.

79. The pharmaceutical formulation of claim 77, wherein the concentration of the copolymer surfactant is 0.001% w/v.

80. The pharmaceutical formulation of any one of claims 77-79, wherein the copolymer surfactant comprises an ethylene oxide/propylene oxide copolymer.

81. The pharmaceutical formulation of claim 80, wherein the ethylene oxide/propylene oxide copolymer is Poloxamer 188.

82. The pharmaceutical formulation of any one of claims 41-81, wherein the formulation has an osmolality of 400 to 500 mOsm/kg.

83. The pharmaceutical formulation of any one of claims 41-81, wherein the formulation has an osmolality of 400 to 480 mOsm/kg.

84. A pharmaceutical formulation comprising: at least one AAV particle, sodium phosphate, potassium phosphate, sodium chloride, sucrose, and a copolymer surfactant; wherein said pharmaceutical formulation has a pH of 6.5-8, and an AAV particle concentration between 1×10¹²-5×10¹³ vg/ml.

85. The pharmaceutical formulation of claim 84, comprising: (i) AAV particles at a concentration between 1×10¹³-5×10¹³ vg/ml, (ii) between 9-11 mM of sodium phosphate (iii) between 1-2 mM of potassium phosphate; (iv) between 90-100 mM of sodium chloride; (v) between 6-8% w/v of a sugar or sugar substitute; and (vi) an ethylene oxide/propylene oxide copolymer; wherein the pharmaceutical formulation has a pH of 7-8.

86. The pharmaceutical formulation of claim 84, comprising: (i) AAV particles at a concentration between 2×10¹³-3×10¹³ vg/ml, (ii) 10 mM of sodium phosphate, (iii) 1.5 mM of potassium phosphate, (iv) 95 mM of sodium chloride, (v) 7% w/v of sucrose, and (vi) 0.001% v/v of Poloxamer-188 copolymer.

87. A pharmaceutical formulation comprising: (i) AAV particles at a concentration between 2×10¹³-3×10¹³ vg/ml, (ii) 1.5 mM of potassium chloride, (iii) 100 mM of sodium chloride, (iv) 7% w/v of sucrose, and (v) 0.001% v/v of Poloxamer-188 copolymer; wherein the pharmaceutical formulation comprises sufficient Tris HCl to provide a formulation pH of 7.3-8.2.

88. The pharmaceutical formulation of any one of claims 41-87, wherein the AAV particle comprises an AAV vector genome and an AAV capsid; wherein the AAV vector genome comprises the polynucleotide sequence of SEQ ID NO: 41.

89. The pharmaceutical formulation of claim 88, wherein the AAV capsid has a serotype selected from AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrb.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, ovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, AAVF9/HSC9, AAV-PHP.B, AAV-PHP.A, G2B-26, G2B-13, TH1.1-32, TH1.1-35, AAVPHP.B2, AAVPHP.B3, AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3, AAVG2B4, and/or AAVG2B5 and variants thereof.

90. The pharmaceutical formulation of claim 88, wherein the AAV capsid serotype is AAV1.

91. The method of any one of claims 1-40, wherein the pharmaceutical formulation comprises the pharmaceutical formulation of any one of claims 41-90.

92. A method of treating Huntington's Disease in a subject, the method comprising administering to said subject a therapeutically effective amount of the pharmaceutical formulation of any one of claims 41-90.

93. The method of claim 92, wherein the pharmaceutical composition is administered via infusion into the putamen and thalamus of the subject.

94. The method of claim 92, wherein the pharmaceutical composition is administered via bilateral infusion into the putamen and thalamus of the subject.

95. The method of claim 93 or claim 94, wherein the pharmaceutical composition is administered using magnetic resonance imaging (MRI)-guided convection enhanced delivery (CED).

96. The method of any one of claims 93-95, wherein the volume of the pharmaceutical formulation administered to the putamen is no more than 1500 μL/hemisphere.

97. The method of any one of claims 93-95, wherein the volume of the pharmaceutical formulation administered to the putamen is between 900-1500 μL/hemisphere.

98. The method of any one of claims 93-97, wherein the dose administered to the putamen is between 8×10¹¹ to 4×10¹³ VG/hemisphere.

99. The method of any one of claims 93-98, wherein the volume of the pharmaceutical formulation administered to the thalamus is no more than 2500 μL/hemisphere.

100. The method of any one of claims 93-98, wherein the volume of the pharmaceutical formulation administered to the thalamus is between 1300-2500 μL/hemisphere.

101. The method of any one of claims 93-100, wherein the dose administered to the thalamus is between 3.5×10¹² to 6.8×10¹³ VG/hemisphere.

102. The method of any one of claims 92-101, wherein the total dose administered to the subject is between 8.6×10¹² to 2×10¹⁴ VG.

103. The method of any one of claims 92-102, wherein administering the pharmaceutical formulation to the subject inhibits or suppresses the expression of the Huntingtin (HTT) gene in the striatum of the subject.

104. The method of claim 103, wherein the expression of the HTT gene is inhibited or suppressed in the putamen.

105. The method of claim 103, wherein the expression of the HTT gene is inhibited or suppressed in one or more medium spiny neurons in the putamen.

106. The method of claim 103, wherein the expression of the HTT gene is inhibited or suppressed in one or more astrocytes in the putamen.

107. The method of any one of claims 103-106, wherein the expression of the HTT gene in the putamen is reduced by at least 30%.

108. The method of any one of claims 103-106, wherein the expression of the HTT gene in the putamen is reduced by 40-70%.

109. The method of any one of claims 103-106, wherein the expression of the HTT gene in the putamen is reduced by 50-80%.

110. The method of any one of claims 103-109, wherein the expression of the HTT gene is inhibited or suppressed in the caudate.

111. The method of claim 110, wherein the expression of the HTT gene in the caudate is reduced by at least 30%.

112. The method of claim 110, wherein the expression of the HTT gene in the caudate is reduced by 40-70%.

113. The method of claim 110, wherein the expression of the HTT gene in the caudate is reduced by 50-85%.

114. The method of any one of claims 92-113, wherein administering the pharmaceutical formulation inhibits or suppresses the expression of the HTT gene in the cerebral cortex of the subject.

115. The method of claim 114, wherein the expression of the HIT gene is inhibited or suppressed in the primary motor and somatosensory cortex.

116. The method of claim 114, wherein the expression of the HTT gene is inhibited or suppressed in the pyramidal neurons of primary motor and somatosensory cortex.

117. The method of any one of claims 114-116, wherein the expression of the HTT gene in the cerebral cortex is reduced by at least 20%.

118. The method of any one of claims 114-116, wherein the expression of the HTT gene in the cerebral cortex is reduced by 30-70%.

119. The method of any one of claims 92-118, wherein administering the pharmaceutical composition inhibits or suppresses the expression of the HTT gene in the thalamus of the subject.

120. The method of claim 119, wherein the expression of the HTT gene in the thalamus is reduced by at least 30%.

121. The method of claim 119, wherein the expression of the HTT gene in the thalamus is reduced by 40-80%.
